SEQUENCE LISTING (138 sequences)

SEQ ID NO: 1 - DNA, 4747 bp, Solanum lycopersicum
  source 1..4747: mol_type = genomic DNA
gtttttcttcaatttcatattctaaaacatgattcaatacatttacccacactggaacctgactaatccgaattcatgaaaattgatgaagaacaaataattctttttcactataaacatacagagatgatgaaacacaagtaaacataaagcaaaagtgagatcaaagcagatctggtgtaatggtagatttatttttattttagacgaataatttttacagcaaccaggtaatgagaagtatcataaaagcaagatgtacactagatttctacagcataggaaaataaaacttcggcacgtttgtacctcatggctgtttagccaaaatcttggagggcaaccaagttataacgcgttcctttaaatgacgattccacttggaatcaaagcatccttgatgacagtgacaatcccactcttgatgaagtatccatctgtttccctagccgcttcttgaacattgtctttgttaatgatctgacaaaagaaaatttattattaggaactcatgctatgaatatggatacatgatatcgagaaaaaacattacacatcaattgcatgttcaaatgacactaagtcagaagtgtaaaagtttaaggaatttaaccttcacattgtcccctatacgagcattcttgtcgataatggctcttttgatgtgacaattcttgccgatgccaattgggacactgccctttgcagccagcagcttcctgtcagcatcagtctgaaccaataatgggtcagaaagcgagtccgttataataagtgaatagagtaaggaaaccacaagctcagtattttctttatcatgtgtagttcgactcttaaggcaagttgctccgcagtaaaccatatacacagcaagtgtaataagcgaagaatgttaaattcatgttcgcgtgtatattagttttagaaatgttttctggtaatcaaagaattattagcattacctcgtagtaatctgcccccatcaaaagtgagtcttctataattgctccctctgatatgcatgatctgagcccaaccacggaatggtgaatcttacagttctgcaaacagaaatattgattaataactatgtagtgctatactatttttctgcattttctggagcactgtatgatgaaaaatgaaaaaaaatggtgaaaagcatattgcattcacttagggtccagaaaaggtccaaacccagggtataatgtagacaacctacccaaagcattaaaggttgattccacgtctcggacctgttaccctctacacacacaagagacaactttaacgttgttccaagactacttcgattctccaatattagtaagtgagttttaaattttgtgcgcctcaacttaatcaaagagccaacttttagatgttatatttcagaccaattgtctctaataagtcaagatctataatatcaacaatatttagccctctccaaccatgatggaaaatcaaactgagaagaaaatgcaaggcaagcatgcctcagagagaagagagtccaatatactttactttatcttctttattatttttgagaaagatggatgaggtcacagaaaatattgaacatgtcagagatatgcctcacatatttgctatcaagactgcaacttagaaatataggagccttgaaacaataactagttaccttgatcacacaaccttcaccaatgacactatctgtgacatcggcatcaagcatttttgaaggtggcaaatatcgaggttgggtgtagattggggctgatcggtcgtaaaagcttcagtaaatattgaagacaacaatattagtgtcaaatcaaagtgagtggataacagcgcaaggtaactatagagaaccatctccttgagatcagcaaaattacctaaaatctggcaccggcttttttgtaatgcccaaattggcattgtagaaagcttcaatggtaccaatatcttcccagtacccatcatataaataagcttgcacctgagaacacagatacataagacttctagaaacaacactaaccaaagtgaccattatcttaaaaagaaattgagcgcgatgcatcattcttttttgagtttccacattgatgaccagaacaagcaaagcaacaaagagaagaaaaaaaataaagaaaaacttgagaagggcaacacacgaaagcagaagacaaaagagagctcatttcataaccataaccatatttggaagagagttcaagaacttgagacagtaaggccctattgctctagagtaaaggtcagaagatgcaactttttttatgcattgaacacagcaaaaagtgtatatttcatattccaactttcatgataagtaaaagtaacatgaagtgtaggttctagagaaagtacatactctcatcccaaatgaagttgcaccaggaataacttcactaccaaaatcattggccccaggaaacttgtcacgaagtaggtttaacatcacgtctttgctaatgacatatatacccatactggcgataaaaggcatttctttagctctcttgtcatcaagacctaaaatggtagtatccacctgaaatacatcaaaacaagaattaacacatgctaataaattctttcttgagtagttctgaattgaaaagcattaggactatgatagtcatactttcattgcttgcaattgctctccttgcggtttctctgcaaattcaataatgcgtccttcttcatcaatcttcatgagaccgaatgcagtggcacgcttctcgtccattggcagtgcggcaacggtaatatcagcatctgtttccctgtgggcttgaataaacttttcataatccattcgatacagatgatctccagcaagtataaggtattcaagaacattatgctcctcaaacaaccacagatattgtctgacagcgtccgcagtgccctacagacaagcatcaacacttgttatgccagtttgtgaagtaaactagttcagagcagcaataaagcataaatagaagtttaatttaaggtgtcatgcacgaatgacacccgagttacaactcaaataatgcatttctaaacagctcttcagaatatacaaagatataggtagtttaagtgctaattgtatgcttcatagtaattgagttgatatttgtatcctcgtataataacaataacagtagttttaagtgaactctatgcagcgggaatgagaatttacctggaaccaatcggggttctccggactttgttgagcagcaagaacttccacaaagccctcgtttttgtatcctcccatattgctagcatatgcccgtgaaaggtggcgatttagagaggcagagttgaattgtgtgagaacatagatcttggatatgttactgttcaagcaattgcttacgggaatgtcaatcagacgataatttgctccaagtggaaccgcaggttttgctctttttttagttagaggataaagtcgggtcccagctccacctccaagaataattcccaaaacactctacattgcataaacaattcaagttcagatcaccgtatttacttagagaaattcagcttcttaaagtttccatatcaaaaaacaagttatcataatacttacttctaagaacacgtgaatcattgtcaagctattaccaaataaggttttgtgaatacaattaacttaatactacaatataactagattgatatactttttatgaaaatataaaaaggttgacatattgaaatggcaatctgcccattattactaatactaacaaataaaaagtgttcagtctaaggattagaaggtgagacacacactactagctatgcctgaaaacgttatttggactctttgaagttgtctttgggtgcgtgtctggtactccaaaagtagtgtattttttaaggatccgacacgctggtgcagcgatgttttgctcatgctcagatagtcagatccaatgactacattttccagaacatattactagttttaacagcttttccaagtcatatcatcaataatgcctatcacgtgcataatgaatctttgaagaatgttggcaaagaattttgttaaatattaaacaaaatgaagtaatccaagcacattttcaaacaggcacgtgatttttgtatcagaaagagggaaaataggtgagaggatctcggatctagaactacccagtcaaaatacttcactctgaagcaacccactccattttttttacaagaggatctacttctccaaaccgtcacaatccaataatcacctcaattaatcagagaaaaaagttctacatttttatctaatgattatatactaatgcatcttatgataaaattgactgaaatttacttaccctgctagcatctgggtccagacatgtctgtgaattctgcgaatcagaaacagccttaggagacacaatcaatggacttcttctcacattgaatcgtactccttgggaacgtaaggacgatacaggcatcaacttgtctccggcgagatgagaagacgaaaatgagagatttctgctggatattgcacgtgtagaatcatttcttctctcattgatgcaattgtgggaagaaggtgaagattttaaggctccaatggaagccgccattgttaacaaagcttgctctcaactatagatactatagagagtgtgtggtagagtgtgattgcacttaattacagctgagatttttggttttataatactgatatcaagggtaactt

SEQ ID NO: 2 - DNA, 1566 bp, Solanum lycopersicum
  source 1..1566: mol_type = genomic DNA
atggcggcttccattggagccttaaaatcttcaccttcttcccacaattgcatcaatgagagaagaaatgattctacacgtgcaatatccagcagaaatctctcattttcgtcttctcatctcgccggagacaagttgatgcctgtatcgtccttacgttcccaaggagtacgattcaatgtgagaagaagtccattgattgtgtctcctaaggctgtttctgattcgcagaattcacagacatgtctggacccagatgctagcaggagtgttttgggaattattcttggaggtggagctgggacccgactttatcctctaactaaaaaaagagcaaaacctgcggttccacttggagcaaattatcgtctgattgacattcccgtaagcaattgcttgaacagtaacatatccaagatctatgttctcacacaattcaactctgcctctctaaatcgccacctttcacgggcatatgctagcaatatgggaggatacaaaaacgagggctttgtggaagttcttgctgctcaacaaagtccggagaaccccgattggttccagggcactgcggacgctgtcagacaatatctgtggttgtttgaggagcataatgttcttgaataccttatacttgctggagatcatctgtatcgaatggattatgaaaagtttattcaagcccacagggaaacagatgctgatattaccgttgccgcactgccaatggacgagaagcgtgccactgcattcggtctcatgaagattgatgaagaaggacgcattattgaatttgcagagaaaccgcaaggagagcaattgcaagcaatgaaagtggatactaccattttaggtcttgatgacaagagagctaaagaaatgccttttatcgccagtatgggtatatatgtcattagcaaagacgtgatgttaaacctacttcgtgacaagtttcctggggccaatgattttggtagtgaagttattcctggtgcaacttcatttgggatgagagtgcaagcttatttatatgatgggtactgggaagatattggtaccattgaagctttctacaatgccaatttgggcattacaaaaaagccggtgccagattttagcttttacgaccgatcagccccaatctacacccaacctcgatatttgccaccttcaaaaatgcttgatgccgatgtcacagatagtgtcattggtgaaggttgtgtgatcaagaactgtaagattcaccattccgtggttgggctcagatcatgcatatcagagggagcaattatagaagactcacttttgatgggggcagattactacgagactgatgctgacaggaagctgctggctgcaaagggcagtgtcccaattggcatcggcaagaattgtcacatcaaaagagccattatcgacaagaatgctcgtataggggacaatgtgaagatcattaacaaagacaatgttcaagaagcggctagggaaacagatggatacttcatcaagagtgggattgtcactgtcatcaaggatgctttgattccaagtggaatcgtcatttaa

SEQ ID NO: 3 - protein, 521 aa, Solanum lycopersicum
MAASIGALKSSPSSHNCINERRNDSTRAISSRNLSFSSSHLAGDKLMPVSSLRSQGVRFNVRRSPLIVSPKAVSDSQNSQTCLDPDASRSVLGIILGGGAGTRLYPLTKKRAKPAVPLGANYRLIDIPVSNCLNSNISKIYVLTQFNSASLNRHLSRAYASNMGGYKNEGFVEVLAAQQSPENPDWFQGTADAVRQYLWLFEEHNVLEYLILAGDHLYRMDYEKFIQAHRETDADITVAALPMDEKRATAFGLMKIDEEGRIIEFAEKPQGEQLQAMKVDTTILGLDDKRAKEMPFIASMGIYVISKDVMLNLLRDKFPGANDFGSEVIPGATSFGMRVQAYLYDGYWEDIGTIEAFYNANLGITKKPVPDFSFYDRSAPIYTQPRYLPPSKMLDADVTDSVIGEGCVIKNCKIHHSVVGLRSCISEGAIIEDSLLMGADYYETDADRKLLAAKGSVPIGIGKNCHIKRAIIDKNARIGDNVKIINKDNVQEAARETDGYFIKSGIVTVIKDALIPSGIVI

SEQ ID NO: 4 - DNA, 3530 bp, Solanum lycopersicum
  source 1..3530: mol_type = genomic DNA
tgtacaaaaaatattcactcatgtctcttcctacttggttaaatctcaagattaagtcccctcatgagtatagatacaagcctacatctaggctcattccatcttctattccatcaaagttgaatctaccatatacaaatcactctgaatgtccaaacattctatgtcctccaatagaccaggtttacatcacattttccttaaatattcacatcttttccctaagatctttactctctacatttgtcgtccttactaaaagtatttgtttcaaaatacttgtcattctttatctatccacttttacttgtttgttcactatactaataataaatgtcacttttccttgtccggttgagaaaaccaagagataattttactctctctgtcccatattagatgggtacttccaactttacatggtgattaagaaatcattaatacattgaaagactttaccattttgccctttgtttatatcaatgcacattaaagtttgggtctttaaaattggtttagacttccaaaggtcatattagtgtagggataaaatggaaagaaaaatagtaattatttctataataatttagtaagtcctcaagtaatatggaacaaacatttttcataagatgccaatttaatatgagacggaaggagtattatttagttccttatttatccttatgattagctatagtcatttttcaaaaaaattagtttttaatattcatagcgtagtatagtaaaattataattctatttattgttccttaagaactgtcaagtaaatattggacaagtaaaactgaacagaggggattctatttttttttctgttttaccattttcgtaatgtgttcttgaactagaaaggggttcactatgaaacttgtccaagtctaatgtagcattgaaaatgaaggggaaagttaggggtcataacgagcgagttcagaattcaattgtcaaggcgtcttttgggttaaaacccaatgggccaaaaccttaaggtcccaaaagttagctgaagagcgagcaataccttgacagttgagcctggacaaatatatcgtgttactattttctactgattttaaaaaaaatatcaatgttccaagaacctataatgtagatttccatgtttactattgttggcagaatgttgcagcaatagtgtttggagatggatcaaattcaaaactttatccattgacaaagaggagatcagaagctgcacttcctatagcaggaaactacaggctcattgatgcagttatcagtaactgcatcaacagtaatatcaagaagatttatgcacttacacagtttaactctgcctctctgaattctcacctttcaagagcttattcctctgcacacatcagaaacgaaccattagttgatgtcattgctgcttatcagagtcctgaaggcaagggctggtttcaggtaaaaaaaaacaggcagtttcaagccttaataaatatgctttctgtaacattcctcattgagtagctgttagcacgaataagagcaacactgttatatccaatgtattctacggataaagaagaagttgagagattttgtaggtttttctctccatcagaactataggatcgttatggttctctatgttttcatgtattctcagggaactgctgatgcgataagaagatgtttgtggattctagaagagtatccgattgtagattttctggttcttcctggttaccatctttacaaaatggatttccaggaactcttaaaggtccacagcaacaacaaagccgatataactgttgcagtgttgagcagaaggacggataaagatgtagaacttggtactttccaagtaaattctggaaatcaagtcatttcgttcaaagacaatccggaggtaaaagtagtacgtttctattttccttctccccgtacatccaattgcttcatttgctcctcattatggactagctgaaaacattattttcaggtacaaaattcagacatttctcatgataagtatgctggtatggatatctatgttgttaacaaagatgccatgataaagcttcttacagagtatcaccccatggccaatgacttcagaagagaagttgtaccaggtgcaatttccttaggaatgaatgtaagagtcgtgtatttccatatccgagcaacttgttgctccaagttttccattttgaacatacgatatctccatcctgtcgcgtaaagatatccaacattttgtcttctactcttacttgaaccattctttggaacaaacaggtatatgcacacaaattcagtggctactgggaggacatgagaaccattgaagcttactaccaagcaaatatggaaagcataaagaacacaaagtacacatataggtcagtatcttcagaaactaaaaagtctcaaagagtaaatcccttaaatatgaactaacggaattttcattttctttagcagcttgtatgacaaagaatctcccctatacactttgcctcgacaccttcctccaactcagataactgatgctgctataacagactctgtgatcggagacggatgctttctcaatgtataaaagtgatcaatttttccttgaaatatagataaagtttatataatttttctcaagaatttggtctcacaaacctctttcttctatacttttgttttccgagtgcagagatgcaagatcagaggtactgtcgtcggcatgaagacaagggtaggagatggagcgataattgaggattcagtaataatgggatctgatacttaccaagtaagacacatgaatacatttcatttggtattcatggatggtcatatagaggaaatggagactatatgaatccagttcatattgaagtaaaattttttgaaagatccctcatatttctacgttcgattcttatcaatcatggtatcaataagaaacattaagtttctagggaaaggatattaacttggtgaaaatatgcagagtggtcgcgtagaggaggatatacacattcctattggaattggagaaggctcacagataaagaaagcaataattgataagaatgtgagaattggcaaaaatgttaaggtatcaaactcaaattgaaccttacacaataaggtttttgaatgcgcgaaaatatcaacaacaaaagatgtacagaatattgtgatttcatatattctttatcaaactgcattcatactttagtgatctttttatgcagatattgaacaaggaaaatgttcaagagtgcaataatgaagcaaatggttacattataaccaaaggcatagttgtaataatgaagggtgccattattccagatgggagcattctgtgacagataatatcattctttgaatgacttgataggaaattatagctacagggagtcaaattgtacactgagattttttttaattcatacattttacttcactttggttgatagttcagatttagatcatacaaaatcattctgtaatgactttaattcatgttaaag

SEQ ID NO: 5 - DNA, 1395 bp, Solanum lycopersicum
  source 1..1395: mol_type = genomic DNA
atgtctcttcctacttggttaaatctcaagattaagtcccctcatgagtatagatacaagcctacatctaggctcattccatcttctattccatcaaagttgaatctaccatatacaaatcactctgaatgtccaaacattctatgtcctccaatagaccagaatgttgcagcaatagtgtttggagatggatcaaattcaaaactttatccattgacaaagaggagatcagaagctgcacttcctatagcaggaaactacaggctcattgatgcagttatcagtaactgcatcaacagtaatatcaagaagatttatgcacttacacagtttaactctgcctctctgaattctcacctttcaagagcttattcctctgcacacatcagaaacgaaccattagttgatgtcattgctgcttatcagagtcctgaaggcaagggctggtttcagggaactgctgatgcgataagaagatgtttgtggattctagaagagtatccgattgtagattttctggttcttcctggttaccatctttacaaaatggatttccaggaactcttaaaggtccacagcaacaacaaagccgatataactgttgcagtgttgagcagaaggacggataaagatgtagaacttggtactttccaagtaaattctggaaatcaagtcatttcgttcaaagacaatccggaggtacaaaattcagacatttctcatgataagtatgctggtatggatatctatgttgttaacaaagatgccatgataaagcttcttacagagtatcaccccatggccaatgacttcagaagagaagttgtaccaggtgcaatttccttaggaatgaatgtatatgcacacaaattcagtggctactgggaggacatgagaaccattgaagcttactaccaagcaaatatggaaagcataaagaacacaaagtacacatatagcttgtatgacaaagaatctcccctatacactttgcctcgacaccttcctccaactcagataactgatgctgctataacagactctgtgatcggagacggatgctttctcaatagatgcaagatcagaggtactgtcgtcggcatgaagacaagggtaggagatggagcgataattgaggattcagtaataatgggatctgatacttaccaaagtggtcgcgtagaggaggatatacacattcctattggaattggagaaggctcacagataaagaaagcaataattgataagaatgtgagaattggcaaaaatgttaagatattgaacaaggaaaatgttcaagagtgcaataatgaagcaaatggttacattataaccaaaggcatagttgtaataatgaagggtgccattattccagatgggagcattctgtga

SEQ ID NO: 6 - protein, 464 aa, Solanum lycopersicum
MSLPTWLNLKIKSPHEYRYKPTSRLIPSSIPSKLNLPYTNHSECPNILCPPIDQNVAAIVFGDGSNSKLYPLTKRRSEAALPIAGNYRLIDAVISNCINSNIKKIYALTQFNSASLNSHLSRAYSSAHIRNEPLVDVIAAYQSPEGKGWFQGTADAIRRCLWILEEYPIVDFLVLPGYHLYKMDFQELLKVHSNNKADITVAVLSRRTDKDVELGTFQVNSGNQVISFKDNPEVQNSDISHDKYAGMDIYVVNKDAMIKLLTEYHPMANDFRREVVPGAISLGMNVYAHKFSGYWEDMRTIEAYYQANMESIKNTKYTYSLYDKESPLYTLPRHLPPTQITDAAITDSVIGDGCFLNRCKIRGTVVGMKTRVGDGAIIEDSVIMGSDTYQSGRVEEDIHIPIGIGEGSQIKKAIIDKNVRIGKNVKILNKENVQECNNEANGYIITKGIVVIMKGAIIPDGSIL

SEQ ID NO: 7 - DNA, 5200 bp, Solanum lycopersicum
  source 1..5200: mol_type = genomic DNA
gatgagcttattcttaaatgaaaatgacagaaatgaaccaaacttttaactaattacatgcattgatgactttttttttcaaagttaaataacatatttaagtcatttttaaaaaccaatatttagttttttcttttgatttctcttaacagtatccatgttaaattccgccaaatattaaattatgaattaataatgatttcgtagataattaatacaagatttattcattttcaactaaaacaaaaaaatctatgcttgagctgattccatccatgtagacttgctataaaaacaaagaatcaaaaatattcttgaacttgaaaaaatatttgttttttcttatattattaatttaatataaacatatgatatgaataaattttgtgtcaaaacttgctataaaaatgtaatcaagtttatatatttttggggggaaagaggagaaaattaagcatgtctatctacaaaggcactactagatgtgctatatatgttagctatgtttttctttgtgttgtccatgaattaattcttgttgtgaagcaccaagaatattctttgtgttacatctcttcctttttttatcattactcttttatctctcttcttatgaaactaaactttaaattcggaattcatactaaaatttctttcacatgagtattaaaatatattatgtgtatatgtttatggctgcgaatgtgaatgaccatgtgatgaatctctattttgttgtggacttaaaagaatagactttatgagatttcaaatatgaagcaatgaaagtaaactttaactaggtcatggattagagatctcgagttcaacttctgataattgaaaaatctctcctcccgtcttactattatgtatatggataatgatagagggaaacgcataatatactaacacgaacgaacagaataattcatatttttttagtaaacattcaaattatattccaggttcattgacgatcactcagaagttccttttaactcattgttgtttggcaggataaattaaaaaataaaggaaactaagtttcaccatcttagatgatggtaccattgggaattactgcatccctaataacggtaacaattccgctcttaataaagtatccgtctgactccctcgctgcttcttgaatattgtcaatattgatgatctgacgacacaaaatttccatgttagagactacctcacagctaatttatgtgatgacaagagaagtcagcagaagttagaagaatgagattttcatcaaacatatataataaaaattgagtggtaataagaataattgatcaagtaaccaaccttcacattgtctcctatacgagcattcttatcaataatagctttttttatgtgacagttcttgccgatgccaattggaacactaccctttgcatacagaagcaatttgtcatcatttgtctggaacaacaaaggaagcaaaccgagtttaacgacaaatcaaaacaaatagtatatgatatctggcgtatatacaaagtaagtctaatagtcatatagtttgtaaatttcaactattacttcataataatctgctcccattaaaagagtgtcttcgataattgttccttctgaaatgcatgatctgagtccaatcacagaatgatgaattttacagttctgtacacaataaagtaacttcatgagaacctaatgtgacaaatatgtactgattaaccaattccaaaaatatgaaaattagaagacaatgttggtgagaaacagttagtaattaattaccttggtgacacaaccttcaccaatgacgctatctgttatatcagcatcaagcatctttgaaggaggcaaatatcgaggttgggtgtagattggagctgaacggtcatagaaacttaagagaatattgaagataacaatattagtgtatactattagagttgaacaataaacaatcgtaaataagtggagagaagtatcagttcacattacctaaaatctggcactggcttttttgtaatgcccaaattagcattgtagaaagcttcaatggtaccaatatcctcccagtagccatcaaataaataagcttgcacctgaacttagactattaatacatctatatctagaagtaaaactaagcaaaaatcatattcaacagaaaaaagttaaaactagaagatagtcacaagtaaaactaagcaaaaattatattcagcagaaaaaagtaaaaacttgaagatagtcacactaaggcctataacttatgttgctcagactcttcaaaaatatcgatagatgccatacgagatactccaaaaatagtacattttggaaagttcgacatgggtatgacaacaatcataaccaatagaatagtgtggaagtaaagtttcataccattctcatgaaacaaaaagtatatttcttctagttttgtgattttagaactataaatggtaaatagatgaataattttacatactcgcattccaatggaagttgcaccaggaataacttcactaccaaagtcattagctgcaggaaagctttgccgaagtaggtttatcatcacatctttgctaataacatatatacccatacttgcaatataagggttctctttagctcgttcatcatccaggcctaaaatagtagtatccacctaaattcaccaaatacaaattacattcttccaagaaacgagatatctgtctttgttcatatatacaatgtgtgtttgttgaaaaatatgcaaaaaggactattgtagcatgtaccttcatttctttcaattgctctccgtctggtttttcagcaaactcaatgatgcgtccttcttcatcaatcttcattagaccaaatgaagatgcacgcttttcatccatcggaagtgcagcaacagtaatatcagcacctgcttctctatgtgcttgaatgaacttttcataatccattctgtacagatgatctcccgctagtattaggaattccaaaacattatgctcctcaaacaaccataaatattgcctgactgcatcagcagtgccctgaaagacaagtagttgcataaaactagtaaaacggctataccaaaacgcatgtcagaagtaaatcagaggagtcgagcacaaattgtaacgttacagtggcttcaaacatgctatatacttgtcggattgtctctaggttataattttattttattttaaaaaaaaagacaaagcatcccgcatagtagggtctagggacaggccgcactccaaagggtgtgccaacctaccccaatgcaagcattagtggttacttacacaactcaaaccaagagacaactttgccggctcattttcaaatcatataactacaaatttaatgttgtttaaaggtgtacattaagaagaatattaggtacctggaaccaatcagggttctctggactttgttgagcagcgagaacttccacaaaaccttcatttttgtgactacccatgttacttgcgtaagctcgtgaaagatggcgatttagagatgcagagttgaattgtgtgagaacgtagatcttggatatggtactgtttatacaattgctcacaggaatatcaatcagacgataatttgctccaagcggaacagctggttttgctctctttttggttagtggatacaaacgcgtcccagctccacctccaagaataatacccaaaacactctaaaacaaattgcattaacaacatagttagttagttaagcatgatcaaccacaaaactttcgttaagaaacaattatgtgttataaatctaaatgaaatgatttatatttacacaaatgtgtcaggtttattttagaccacaaatttcaaaaaaaaatctttcttaaatattatgtctaacaaacattgtcacataattggacgaaacactgcatatctacctaaatatgggtgaaatatgaatgaaataatttatactcatacaaatatgttagatttattttagatcacaaatttcagaacttttttcttttcgaaatattatgtctaacattgttagataacttgggagggagagattgaaatacaaactatatgataatttatataatcaaaattttcaaaatacattgaatgcgattcaaatttaagtataaaatttataaaacttatgacaaaacatggaaaagaaaaaagtgtttaattttgcactaacttggctagcatctggatcaagacatgtctgtgaattcgacgagtcagaaacagctttaggggacaccataatagcagcccttttatgataagccttcttattggaactgaatcgtctagaacaaaatgacaatggtgacatcaatttatctccggataattgtgacgatgcaaaagagagactttgaaaggacattgcaggatcaacattctcgattataggttggcgaaaaggtttcaaagttcccatggacgctgccattgtagtaccactgcccttgcctattcttccagcttctcactgtgtgctattttcaatgaaatttgagaaaagttttactcaaaaacaagtcaatataatataattattaacctaaaaataaaacttaaaaatattttttaaaataaataaaaaacttgatctgatttggtctcttatttgaacaattcaaaccaaactctaggtattgagaccttttatttatgataataaatattaatttattatatataattaaatattattttcaagtcattttatcatacaattttaccatttattgtacagtgatacataaatataaaattttgttttcccatattatcaaatttttattccaatatgtcaaattataattaatttaacttatttgcaaaaaacttactataacctctaaaaaaatcactactctattatatataaatttaattttacaagttgataaaatatgttagccaattattgcaatacaatatcttggatattagtctaaaaaatcttattgatagacgatgggattaaggtaattagattcaaaattttaaaatatcaaataaagttttaatgttattaaagtaaaatgtttaaaatttgaaaagcaaaatttaatacattcctaatacatgggcaaagtagtagcttgaaaatcttccagcaagaacgagacagcgtgacatacacaacaacaaccaaaagaaaaagctttatagtt

SEQ ID NO: 8 - DNA, 1560 bp, Solanum lycopersicum
  source 1..1560: mol_type = genomic DNA
atggcagcgtccatgggaactttgaaaccttttcgccaacctataatcgagaatgttgatcctgcaatgtcctttcaaagtctctcttttgcatcgtcacaattatccggagataaattgatgtcaccattgtcattttgttctagacgattcagttccaataagaaggcttatcataaaagggctgctattatggtgtcccctaaagctgtttctgactcgtcgaattcacagacatgtcttgatccagatgctagccaaagtgttttgggtattattcttggaggtggagctgggacgcgtttgtatccactaaccaaaaagagagcaaaaccagctgttccgcttggagcaaattatcgtctgattgatattcctgtgagcaattgtataaacagtaccatatccaagatctacgttctcacacaattcaactctgcatctctaaatcgccatctttcacgagcttacgcaagtaacatgggtagtcacaaaaatgaaggttttgtggaagttctcgctgctcaacaaagtccagagaaccctgattggttccagggcactgctgatgcagtcaggcaatatttatggttgtttgaggagcataatgttttggaattcctaatactagcgggagatcatctgtacagaatggattatgaaaagttcattcaagcacatagagaagcaggtgctgatattactgttgctgcacttccgatggatgaaaagcgtgcatcttcatttggtctaatgaagattgatgaagaaggacgcatcattgagtttgctgaaaaaccagacggagagcaattgaaagaaatgaaggtggatactactattttaggcctggatgatgaacgagctaaagagaacccttatattgcaagtatgggtatatatgttattagcaaagatgtgatgataaacctacttcggcaaagctttcctgcagctaatgactttggtagtgaagttattcctggtgcaacttccattggaatgcgagtgcaagcttatttatttgatggctactgggaggatattggtaccattgaagctttctacaatgctaatttgggcattacaaaaaagccagtgccagattttagtttctatgaccgttcagctccaatctacacccaacctcgatatttgcctccttcaaagatgcttgatgctgatataacagatagcgtcattggtgaaggttgtgtcaccaagaactgtaaaattcatcattctgtgattggactcagatcatgcatttcagaaggaacaattatcgaagacactcttttaatgggagcagattattatgaaacaaatgatgacaaattgcttctgtatgcaaagggtagtgttccaattggcatcggcaagaactgtcacataaaaaaagctattattgataagaatgctcgtataggagacaatgtgaagatcatcaatattgacaatattcaagaagcagcgagggagtcagacggatactttattaagagcggaattgttaccgttattagggatgcagtaattcccaatggtaccatcatctaa

SEQ ID NO: 9 - protein, 442 aa, Solanum lycopersicum
MQCFVQLCDNSVLGIILGGGAGTRLYPLTKKRAKPAVPLGANYRLIDIPVSNCINSTISKIYVLTQFNSASLNRHLSRAYASNMGSHKNEGFVEVLAAQQSPENPDWFQGTADAVRQYLWLFEEHNVLEFLILAGDHLYRMDYEKFIQAHREAGADITVAALPMDEKRASSFGLMKIDEEGRIIEFAEKPDGEQLKEMKVDTTILGLDDERAKENPYIASMGIYVISKDVMINLLRQSFPAANDFGSEVIPGATSIGMRVQAYLFDGYWEDIGTIEAFYNANLGITKKPVPDFSFYDRSAPIYTQPRYLPPSKMLDADITDSVIGEGCVTKNCKIHHSVIGLRSCISEGTIIEDTLLMGADYYETNDDKLLLYAKGSVPIGIGKNCHIKKAIIDKNARIGDNVKIINIDNIQEAARESDGYFIKSGIVTVIRDAVIPNGTII

SEQ ID NO: 10 - DNA, 4243 bp, Solanum lycopersicum
  source 1..4243: mol_type = genomic DNA
cataccttgttgtttccaacttcctagtcctcaagtttcatcacatcaaccttcatatatcaaaaaaaaaaatacacaaatataaacagccatttcagtactcttgattctcagaggaaaaaaaaatgtctgtggctactgacgggagattcgcgctacttcgtaataaccctgcagcactgacagggacaaacttgaagatagtgaagttttgcaatggagaattgatggggaagaaactcaagtatacaaaatttcagttaaggagtaatgtagtgaagcctaatatctgcatgtcacttacaactgatattgcaggtgaggctaaggtgagtcttctatatatataaatatctttgcttcatttacaattttgaatgaagcattggtgactactgtatttaataaaaatatgggccacttctcttcataaaacaagttagatcaaatgagccacttaactaattacgccatatcattatgatgtagtattggaatgtaacttaaaaatcttgaatcagtctccaactttacctcttaaaatggccattttgactaactgtcaaatgtggacagttgaaggatcttgaagcaaagaaggaggatgcaagaacagtagtagcaatcattctaggagggggagcgggaactcgtcttttccccctcaccaaacgtcgtgctaagcctgctgtaagttttcttttcagataatcagagaaaatattactacgagtgcttgcagagttatctgcatctcaccaatgagtcattatcaatttgcttcaggttccaataggaggagcatacaggctaattgatgtaccaatgagcaactgtattaacagtggcatcaacaaagtatacattctcactcagttcaactcggcctcacttaacaggcatattgctcgtgcttacaactttggcaatggggtcacattcggagatggttatgtcgaggtaaacatacaacaccaattctcatttacttagtttcaggttttgatgttattggcagcaccatggctgataagttgtagtacaggtcttagcagcaactcaaacaccaggtgaattaggtaaaagatggttccaaggtactgcagatgctgtgaggcaatttcactggctttttgaggtacacacatgatcacattgaattgcaatatccataatttagtaatttaccctaataacagtgcaaacatcaatatattccagaactgcaatatttctgaagaaaaatcgaaatgcatatgttcatacatcataactggagatttcaattatagtcgacctgaatataattcttgatgacttgatggcatagaagtaaactaaaggtccactaatagttttcatattcaacaaattaccaatgcaagatcaagttagctaagttgtataagcaatactttttctacactcagctatagccaagccaaaattatctcattctatgggttagtcacaaacatgaaactaattctagtaaacattgggtatgcaatgttcacactgattttccataacttaaggatgcaagaagcaaggacatagaagatgtgctcattctctctggagatcacttgtatagaatggactacctgcactttgttcaggtgaagactagactaagctataagtacttaccttgattgatatcatatatctgaaattacctataactcctcattgatatttccatctccttgacagagtcatcggcaaagtggtgcagacattaccatatcaagcttgccaatagatgacaggtagcattaggagggataatctagatactacataggctatttcttcctctcaacgatggcatactttctcaaagactgcttgaaaatcacaacaagacataatttcttctattgaatcacttctatgttttcatgatagtcgagcttcagattttggcttgatgaaaattgatgacacagggcgggtcatgtccttcagcgaaaagccaaaaggagatgatttgaaggcgatggtaattcttgaaaatttgacagtctcagagcttaatctactttaaattttaaagttgttgcaatcacttgacaacaagtacttcaggcagtagacacaactgttctgggattatctccagaagaggctaaagagaaaccttacattgcttcaatgggagtttacgtcttcaagaaggatattctcctcaatcttttaaggtatgtaacgagtttcttctatagtacccttatttagagagtgtcatggtcctttcaacatgcaaatgagagaagctctggaaaaaggacaaaaaaagtcaacaaattatatgtatccagcttaataggggtagctacactcaagagaagcaaattgtggtgcttattataaagcagaaagagaccgcataaaatcttatgttaaccccgagttccctcatactttatctatagtgacatgctgaagatgtcaggtaaaagaagatcatgaaatgactactctttgtctttttagatggcgttttccaactgcaaatgacttcggttcagagatcatacctgcctctaccaaagaattctgtgtcaaggtgtgtaccacttaatttctggtatgcatttatgcaggaccttatcagttcacaattcctctccccctctattatcttattccaccccaccccaccccccagccccccccccccggcatgtctttaacgtcaattcactatttcggttatggttccctaagcagcttatcttcaacctgtttgcttgattttgcaggcttacttgttcaatgattactgggaagatattggtacaatcagatccttttttgaagcaaaccttgcactcactgaacatgtaagaaccggagagctattatatgatttttatagtctttaaagggtttattttttcaaacaacctgagtaagatggaatatggccattgaacaccagtcaatcttatttgacaatattaaaacgatggttcgtactaatcatacggccaactatgtttttttctttttcttccagccaccaagatttagtttctatgatgcaacaaagccaatatacacgtcaaggagaaacttacctccatcagcgattgataatagcaaggttcaagacacagacccacgtcttcaatcttaaacagatacatctttaacttttcttctactaaccaaatctatttgttgccagattgttgattcaattgtatcacatggtagtttcttgaccaattgcttcgtggagcacagtgttgttggcatccgatcccgtataggcaccaatgttcacttgaaggttagtctagtaaaacagctattgaaagttcagtattatgcttttgcacaacaaaaccaaaaattggttctccaactagattccacgtagatccaacacaagaacattcgtgtttcacattgaaacaattagaacataccatacaaacatgttaagtacttctacgtttacttgattttgtaattgctgctaattcagcatacacttggttgaaatccatataggacacagtgatgcttggtgctgactactatgaaactgatgctgagattgcttcacagctagctgaaggaaaagtgcctttaggaataggagagaataccagaataaagtaaatcactaagattgttctccttcagtgtttatcaagcaagaaaatgaatgtgattctaattttatttctcacgttatttcaactaatatgcagagagtgcatcattgacaagaatgcaagaattggaaaaaatgttgttattgccaactcagaggtatgacctcccatactcccaaaaacagcaagagatcaaattttatctttttattactgggagtcccttattttcctctgacacatcaatttgcagggtgtgcaagaagcagacagatcctcagaaggattttacatacgatcaggcatcacagtcatattgaagaactcaacaatcccagatggaactgtgatatgacaagtgtgacaatgtctttttgtcttctctttcaagtagaattggcaccaagaaaattgtgaaatatgagaagtaaaagtttcagattatcaacttttccacatgcagcaagtaaatattgaactaaaggccgatgataagaagggttgtttctcccattgatgaattttgaactaagaataaaatgtctgttcaagggtgaactttttcaataaactagctgatgacactaacaaaatatggttttcacagt

SEQ ID NO: 11 - DNA, 1548 bp, Solanum lycopersicum
  source 1..1548: mol_type = genomic DNA
atgtctgtggctactgacgggagattcgcgctacttcgtaataaccctgcagcactgacagggacaaacttgaagatagtgaagttttgcaatggagaattgatggggaagaaactcaagtatacaaaatttcagttaaggagtaatgtagtgaagcctaatatctgcatgtcacttacaactgatattgcaggtgaggctaagttgaaggatcttgaagcaaagaaggaggatgcaagaacagtagtagcaatcattctaggagggggagcgggaactcgtcttttccccctcaccaaacgtcgtgctaagcctgctgttccaataggaggagcatacaggctaattgatgtaccaatgagcaactgtattaacagtggcatcaacaaagtatacattctcactcagttcaactcggcctcacttaacaggcatattgctcgtgcttacaactttggcaatggggtcacattcggagatggttatgtcgaggtcttagcagcaactcaaacaccaggtgaattaggtaaaagatggttccaaggtactgcagatgctgtgaggcaatttcactggctttttgaggatgcaagaagcaaggacatagaagatgtgctcattctctctggagatcacttgtatagaatggactacctgcactttgttcagagtcatcggcaaagtggtgcagacattaccatatcaagcttgccaatagatgacagtcgagcttcagattttggcttgatgaaaattgatgacacagggcgggtcatgtccttcagcgaaaagccaaaaggagatgatttgaaggcgatggcagtagacacaactgttctgggattatctccagaagaggctaaagagaaaccttacattgcttcaatgggagtttacgtcttcaagaaggatattctcctcaatcttttaagatggcgttttccaactgcaaatgacttcggttcagagatcatacctgcctctaccaaagaattctgtgtcaaggcttacttgttcaatgattactgggaagatattggtacaatcagatccttttttgaagcaaaccttgcactcactgaacatccaccaagatttagtttctatgatgcaacaaagccaatatacacgtcaaggagaaacttacctccatcagcgattgataatagcaagattgttgattcaattgtatcacatggtagtttcttgaccaattgcttcgtggagcacagtgttgttggcatccgatcccgtataggcaccaatgttcacttgaaggacacagtgatgcttggtgctgactactatgaaactgatgctgagattgcttcacagctagctgaaggaaaagtgcctttaggaataggagagaataccagaataaaagagtgcatcattgacaagaatgcaagaattggaaaaaatgttgttattgccaactcagagggtgtgcaagaagcagacagatcctcagaaggattttacatacgatcaggcatcacagtcatattgaagaactcaacaatcccagatggaactgtgatatga

SEQ ID NO: 12 - protein, 515 aa, Solanum lycopersicum
MSVATDGRFALLRNNPAALTGTNLKIVKFCNGELMGKKLKYTKFQLRSNVVKPNICMSLTTDIAGEAKLKDLEAKKEDARTVVAIILGGGAGTRLFPLTKRRAKPAVPIGGAYRLIDVPMSNCINSGINKVYILTQFNSASLNRHIARAYNFGNGVTFGDGYVEVLAATQTPGELGKRWFQGTADAVRQFHWLFEDARSKDIEDVLILSGDHLYRMDYLHFVQSHRQSGADITISSLPIDDSRASDFGLMKIDDTGRVMSFSEKPKGDDLKAMAVDTTVLGLSPEEAKEKPYIASMGVYVFKKDILLNLLRWRFPTANDFGSEIIPASTKEFCVKAYLFNDYWEDIGTIRSFFEANLALTEHPPRFSFYDATKPIYTSRRNLPPSAIDNSKIVDSIVSHGSFLTNCFVEHSVVGIRSRIGTNVHLKDTVMLGADYYETDAEIASQLAEGKVPLGIGENTRIKECIIDKNARIGKNVVIANSEGVQEADRSSEGFYIRSGITVILKNSTIPDGTVI

SEQ ID NO: 13 - DNA, 5124 bp, Solanum lycopersicum
  source 1..5124: mol_type = genomic DNA
tgattcacgttaatatgatggactctgtaaatctcaagagagtccacagactttacaacaacacagaaatctcaagagagtccacagactttacaacgccactgtgttgtgttatgaacgcactgtaacctagtaataattttgtttataccaaatactttttgaggaaaagttgggtgtttgaaaaagcttaattcaggtactgagaaagctgtttatatatatatatatagatagatagatagatagatagaaaaatgatagttgtattttgtatagattctgcaaaagttgtatgatttgattgttttttgatctagtttaataaaaagggttttgggtaatgtaagttatttgatttggtttgttcctttggaatcaatatattgatattttggggttgcaagttgtgttttgtgattattctattggaatctatgaattgatattttagtacaggaatctgataccgtgtaaaataaaatgttttttgcacttcctgtgtaaagtgaaatcttttgctttttgtttttgttttttagaatgaatgtacttgcttatgttgtggttgacgtgaataattgatagagcaatggatgcgttgtgtgcgggcactgcacaatcagtggcaatttgcaaccaagagagcacattttggggacagaagattagtggaagaagattgataaacaagggttttggggtccggtggtgcaagagctttacaactcaacaaaggggtaaaaatgttacctctgctgttctcacccgtgatataaacaaagagatgctggtaagtagaggaattaggtgataaagtaaagtgaatgatcaagaacaagattaatttgtattattcatgttgtttttcagccatttgaaaattcaatgtttgaggaacaaccaacggcagatccaaaagctgttgcctctgtcattctaggtggtggtgttggaactcgtctttttcctcttacaagcagaagagctaaaccagctgtaagagttacaacacacacatatatatattatatatctctctttttgcttgaatcgtggttgttactccattatgtgtcttttgagtttgacaacatgtccaattcataccaacttcagtcttaaaaaattgtagtggttcttaaatccatttcaattggatttttgacaactaaagaaatgactagttaagctaagtgtggattcccttctctgttctcaatgcaactaagcttaagaatgcattctctatcagttttcattcaaattctgtgactatcggctttgttcttcaggttcctattggtggttgttaccggctaattgatgtaccaatgagtaactgcattaacagtggcatacggaaaattttcatcttaacacagttcaattccttttccctcaatcgtcaccttgcccgcacgtataattttggaaatggagtgggttttggagatggatttgtggaggtaagatcattttggttacttaaagtttttagtttgagctcttttaagctacttatattttgacattgaagttcaatcatatctttgtaagaaatcatatctcctcttgctcctttggatcctgggggaggtagatctacaaacctaaaatggggaagataactgaaatgtgtccttgacagataaaaaagattttctcaaatgcaggttttagctgcaacccagactccaggggatgcaggaaaaatgtggttccaaggcactgctgatgctgtgcggcaattcatatgggtatttgaggtacacaaataagaccggtcattgataaagctccctaggaaaagcgctgaccttattattttttaaataatgacatagaatcagaagaacaagaatgtcgagcacataataattttgtctggtgaccatctttaccgaatgaactacatggactttgttcaggtatatgagcaacttatgacaacaattgcttcctacaccaaccctgtgttcatatactttgcttaatgaataatatttgttcttgcagaagcatattgacgcaaatgctgatattacagtttcatgtgttccaatggacgatgggtatgagattgatctttcatttaacttgatagcttatttaatgtgaagccaaacaatatttgcattgctttctatctccctgaactctttgaaaggttcttcttagtgttgttcagagtggcaaatggtgaccacatttgtttagaatctgaaggcatattgcaaattgaagtggcactatgtatctgatatatcaatctacatataaaattgtacaaggccctatttcatcatgatttgtatgaattctcaatatggttttggctttcagatcaggaaatagaataagacagaatattagaacagtagaaagcttgaaattctttttagtttagctatttaactggacctggtatgtatttaatcaccatcatcatggatacatagtaataatgtagatacattttcccatcttgcatcctcattaacttctaagcaccagcactgattaatatccactgaaagatctggaagcatacagaggtagactttgcagtttcccttaagtctatttgtgttatctgcttccagatttcagtcatgttgtctatttagaatgcactattagggctaacaaacatttttaagctctatcaaggagttaatcaagacccttctctctctctgtctcttaccctcttccatcctctcttttggcagtcgagcttcagattttggattgatgaaaatagatgaaacaggacgtatcatccaatttgctgaaaaaccaaaaggccctgctttgaaggcaatggtacatatatttgggcttccacacctggaactttgtaaattctttttaaactggattagctattaaaaaattatccccttacagcccctcttactttcttattgtctcttctcctacataacttgctttgtatgtttaatattctattacagcaagttgacacatctatcctcggactatctgagcaagaggcgtccaattttccttacattgcatccatgggtgtttatgtattcaaaactgatgttttactaaaacttctcaagtcagcgtacccttcatgcaatgattttggctctgaaattatcccttctgctgtgaaagatcacaatgtccaggtaatagctggtggtaactttctcaattgcaacttctagccttttgaagttcacaaattttgctgttcattgtattctttgattctgactgattgattttattaggcatatttgtttaatgattactgggaggatattggaacagtgaagtctttctttgatgccaacttggccctcacaaagcaggtgagcaccttaatgtttactaccatctaactacttctaccattactcgaagtaagagctagacggttgatgcaaggactctgaaatatgtagcagtattttaagtaatcgtcagcgaatttggtagttctatttctttttaatttaccataatctccatcttcagttattgattaataacttgttactaaattgcatttttcctcttttatccctgtttaatgtagcctccaaagtttgacttcaatgacccgaagaccccgttctacacttctgctaggttcttacctcctacaaaagttgacaaaagcagggtatgcaagttaacataaatacttgtaataaaaatttccaggattgctgggcgctatgaacttgtcatgttaatgctgttcattgtcagatagtggatgcaataatttcacatggctgctttctgagggaatgtaacattcagcactccatagttggtgtacgatcacgcttagactatggtgttgagtttaaggtattgtttcatactcatttaaagcttttcttatctgtttttaggttgacagttctatgagctagtgcttggactaaattaaaaacgacatcctgcattcatctgatgcattctgacaacttagaaacctagatgacagacttgtaaggcacataacagttaccatctacctgtgaatgtgcaatcaaactagttgtaggaaggatgatttacacgttttgatttatttctgcaataagctggtaacatgtaccctaggtatggactttagcatattacagtaatgaacaagtataacctcagcactcaggtgctttgttcataaacagctatttagtgcttacttgtttataggctgaatgtgtctttacatttatcaggataccatgatgatgggtgcagattattatcaaactgaatccgagattgcttcactattagcagaaggaaaggttccaataggggtcgggccaaacactaagatacagtaagattttctactgaactcgtactcacttctcacacttgtggattgaaaattgaccactcagacttccattttgaaggaccagcttaaaaactgagctctggtcctctgattagtgtatcgttattcatttcagtgtagtttacagttctttgcccaaacttcaacaaggttaccttgagtggactttgacatgcttttctttgtattttcttgaaggaaatgcataattgacaagaacgcaaagataggaaaagatgtggttattctgaacaagcaagcaagtatcttttttttgaagtttgaaatcatcacgaaagttaatattagtttaactgatgcttggatgttgttgcagggtgttgaagaagctgataggtcagcagaaggattttacattaggtccgggattaccgtcataatgaagaatgcaaccattaaagatgggacagtcatatgaaatcttgctaagtgttcttgctagttgggaaataaagtaggctggtatggtctagtgatatgaaaactagtgatatgatgaatgtgctactagggcttttctgacattgtttactttctacctatgacatattcactttcttggacttgatgaaacacatttccaagtgaacctcagtcctccatcttttaagtgttcagctgccagcagcttagtgagtgtctttgccgcctcgcttcagtttccaccatttacatagggggtttgacatggggtttgctggaaaattatattgtatatataggttagatattac

SEQ ID NO: 14 - DNA, 1557 bp, Solanum lycopersicum
  source 1..1557: mol_type = genomic DNA
atggatgcgttgtgtgcgggcactgcacaatcagtggcaatttgcaaccaagagagcacattttggggacagaagattagtggaagaagattgataaacaagggttttggggtccggtggtgcaagagctttacaactcaacaaaggggtaaaaatgttacctctgctgttctcacccgtgatataaacaaagagatgctgccatttgaaaattcaatgtttgaggaacaaccaacggcagatccaaaagctgttgcctctgtcattctaggtggtggtgttggaactcgtctttttcctcttacaagcagaagagctaaaccagctgttcctattggtggttgttaccggctaattgatgtaccaatgagtaactgcattaacagtggcatacggaaaattttcatcttaacacagttcaattccttttccctcaatcgtcaccttgcccgcacgtataattttggaaatggagtgggttttggagatggatttgtggaggttttagctgcaacccagactccaggggatgcaggaaaaatgtggttccaaggcactgctgatgctgtgcggcaattcatatgggtatttgagaatcagaagaacaagaatgtcgagcacataataattttgtctggtgaccatctttaccgaatgaactacatggactttgttcagaagcatattgacgcaaatgctgatattacagtttcatgtgttccaatggacgatggtcgagcttcagattttggattgatgaaaatagatgaaacaggacgtatcatccaatttgctgaaaaaccaaaaggccctgctttgaaggcaatgcaagttgacacatctatcctcggactatctgagcaagaggcgtccaattttccttacattgcatccatgggtgtttatgtattcaaaactgatgttttactaaaacttctcaagtcagcgtacccttcatgcaatgattttggctctgaaattatcccttctgctgtgaaagatcacaatgtccaggcatatttgtttaatgattactgggaggatattggaacagtgaagtctttctttgatgccaacttggccctcacaaagcagcctccaaagtttgacttcaatgacccgaagaccccgttctacacttctgctaggttcttacctcctacaaaagttgacaaaagcaggatagtggatgcaataatttcacatggctgctttctgagggaatgtaacattcagcactccatagttggtgtacgatcacgcttagactatggtgttgagtttaaggataccatgatgatgggtgcagattattatcaaactgaatccgagattgcttcactattagcagaaggaaaggttccaataggggtcgggccaaacactaagatacagaaatgcataattgacaagaacgcaaagataggaaaagatgtggttattctgaacaagcaaggtgttgaagaagctgataggtcagcagaaggattttacattaggtccgggattaccgtcataatgaagaatgcaaccattaaagatgggacagtcatatga

SEQ ID NO: 15 - protein, 518 aa, Solanum lycopersicum
MDALCAGTAQSVAICNQESTFWGQKISGRRLINKGFGVRWCKSFTTQQRGKNVTSAVLTRDINKEMLPFENSMFEEQPTADPKAVASVILGGGVGTRLFPLTSRRAKPAVPIGGCYRLIDVPMSNCINSGIRKIFILTQFNSFSLNRHLARTYNFGNGVGFGDGFVEVLAATQTPGDAGKMWFQGTADAVRQFIWVFENQKNKNVEHIIILSGDHLYRMNYMDFVQKHIDANADITVSCVPMDDGRASDFGLMKIDETGRIIQFAEKPKGPALKAMQVDTSILGLSEQEASNFPYIASMGVYVFKTDVLLKLLKSAYPSCNDFGSEIIPSAVKDHNVQAYLFNDYWEDIGTVKSFFDANLALTKQPPKFDFNDPKTPFYTSARFLPPTKVDKSRIVDAIISHGCFLRECNIQHSIVGVRSRLDYGVEFKDTMMMGADYYQTESEIASLLAEGKVPIGVGPNTKIQKCIIDKNAKIGKDVVILNKQGVEEADRSAEGFYIRSGITVIMKNATIKDGTVI

SEQ ID NO: 16 - DNA, 4634 bp, Solanum lycopersicum
  source 1..4634: mol_type = genomic DNA
tgagagttcacaagccaataaagctgatcacactcccctttgtccacttgatcactcaataacacaacttcttgtgattcacttacaattctagatctcctgaaaaactttttcttgattctacaccactagcaatttacattttctctttcactgattttggttacttatttgacattcttgttttcaagatccaaaaatcatcactttccaggttcaaaatcttgttttttttttaattttgtcttttaattgctgtatattgtggaactccacaggtgaatttttatgatatgagttcaccctttagctttctttgtaatatgagtgaatttttatacatatattttctttgtatcaattttagaaaatatatgtataaaatacctaattttatgtagagattcatgtttttgtggtaatgctatgaggtattagtactgagcatatagctagcttgggttttgggtttaccgaccttttttttttttaattagtgatattttcttttatgtattttatacttttcttggttgcttagaaattacacatactttattgagatttgaataaatctatttgatttggatccgttgatatgggattgctgatttgttgattggctgcagaaatggatacttgttgtgcggctatgaaatcgacggttcatttggggagagtgagcactggtggctttaacaatggagagaaggagatttttggggagaagatcagagggagtttgaacaacaatctcaggattaatcagttgtcgaaaagtttgaaacttgagaagaagattaaacctggggttgcttactctgtgatcactactgaaaatgacacagagactgtggtaataatcaattcttttctctttattaggacatgtctgcttcaaaataatgccataatagtgttgtagatatggtggaaacaatctcatatgaactatattgtgaatggaagtgaatccaataattaagattgttagcattgaactcaccacacttaaaagattatgggttgtgagtttaattttgttgaagttctattactagtcttctgctatatatttgtttcgtgttgagaagatattgtgttcggttaagcctacaatatttatctgcatcagtctgtgattgtgattgtttgttggacttatgtagttcgtagatatgccacgtcttgagagacgccgggcaaatcccaaggatgtggctgcagtcatattaggaggaggcgaagggaccaagttattcccacttacaagtagaactgcaacccctgctgtaagagatttatctttgctcatgaaagttttatcaattttttgttttatatgcttaatgtccagttaaaactctcttccaagttaattttgtttatctcgtgttaaatttcagattttttttttaatgatatgttattttttgatttctacatcgattaaatatagatagaataggtgtacaagattgcaagtcaatttcttaagattctgatgttttgtgcaacatggcaccaggttccggttggaggatgctacaggctgatagacatcccaatgagcaactgtatcaacagtgctattaacaagatttttgtgctgacacagtacaattctgctgccctgaatcgtcacattgctcgaacgtattttggcaatggtgtgagctttggagatggatttgtcgaggtttgagactatacttctggattagtcttgctagatatgacagctgagctaattaatatgagtaaatttgctggtcctccaagtgatttgttcacaaaaaatctgtaacaatttctgatgattattttctttttttttctgctttcttgcttcgttaactgaaaggaaatccgtcattaaacaggtactagctgcaactcagacacctggggaagcaggaaaaaaatggtttcaaggaacagcagatgctgtcagaaaatttatatgggtttttgaggtttgtctctacaaatctttaggacactaatccaccactgctctcttctgtcgaccggatatgatttctgaaaaatgtctcattattttttcttgaatcagttgttatgtacagacatatactacttttgtgtttacaaggcaaccaattcttgcaggacgctaagaacaagaatattgaaaatatccttgtattatctggggatcatctttataggatggattatatggagttggtgcaggtatgtcaactttaactacctacacactcctcattctttgtttgcctttttttttcttttaaaaaacaaagtttctgactgcctccgatcttcaaacagaaccatattgacagaaatgctgatattactctttcatgtgcaccagctgaggacaggtttgtttaaattcttataaggaacaactagtataggttaagtgaattgagtgagatcagaaacaagatggagaaagaatgcttctcctctaatactcatatgcatacgctcgctatacattacttcgaagtaaaaacatcaacacattttttaataccttattgtgattatgttaagctgagatttaagctttacttatcagccgagcatcagattttgggctggtcaagattgacagcagaggcagagttgtccagtttgctgaaaaaccaaaaggttttgagcttaaagcaatggtaggtttagttgaaatctttcttttagtatatgtgctgatttctttctaaacaagtttttgtggtttatattcagactagtgttcctttagaatttaactcatttatttcttgtgctgcagcaagtagatactactcttgttggattatctccacaagatgcgaagaaatccccttatattgcttcaatgggagtttatgtattcaagacagatgtattgttgaagctcttgaaatggagctatcccacttctaatgattttggctctgaaattataccagcagctattgatgattacaatgttcaagtaaaggatcttggatctttcgattgaaattatctttatgaattgagtacattagttgctcaaacgaaaaattagttgtagttttaacgaatttatgctatcttctcaggcatacattttcaaagactactgggaggacattggaacaattaaatctttctataatgctagcttggcgctcacacaagaggcatgttgcaaattcatctttatacttctaacgactagctcagaaattattttacatcagtgcataatccaactgattaatttccattgcagtttccagagttccaattttacgatccaaaaacacctttttacacatctcctaggttccttccaccaaccaagatagacaattgcaaggtaagtgggtaaatactgcaagtgttttgcattaattggtcttttgtgggttctaataagttttgttttctgtgctttttcagattaaggatgccataatttctcatggatgttttttgcgagattgcactgtggaacactccatagtgggtgaaagatcgcgcttagactgtggtgttgaactgaaggtttgcgtgtagccctaatttttatcttcctttaaaactattcctgaacaaacacatttattttctttagtttgtgaaaaatgcagcagcttattgtttctagtacattttgttgtaaagtgtagatctctaacacattttacaaccacaggatactttcatgatgggagcagactactaccaaacagaatctgagattgcctccctgttagcagaggggaaagtaccgattggaattggggaaaatacaaaaataaggtaaatgaacattgaatattatctttttttgacaaatgtatttcattttcatacttgagaattctttaatttgattatggtttcactcatacaggaaatgtatcattgacaagaacgcaaagataggaaagaatgtttcaatcattaacaaagatgtaagttttttttttgcatcctccgactttgtttagtgtgtcttattttcatagtacttatagggcggaggttagtttcccaacaactattgttcacacagtcaagttcaaaacacaaattgctggtgtgcttcttgctcgcgagaaagaaagcagaagagtgttcaattcagatttgattgtttgtagcatgggagcacttatatttttgagacataattgattgaatgagactccctgatgttcttttctttgaaatatatttagggtgttcaagaggcagaccgaccagaggaaggattctacatacgatcagggataatcattatatcagagaaagccacaattagagatggaacagttatatgaactagaaaagcatctcttgttgaactagtggaaatccaaatctcaacttgaagaaggtcaagggtgatgacctattttcccacgaaaacctagcacattcagctccggaagaagctttctatgctcatgtgaaaagagcaagtataatcgagacacgtgtctaaaataaaactaaagttgctgcctagttcatgttctaagagtcacattatgtaagatagcattttgaagaactcaatagtttatctattgctcaacttaggcttttcgtattgtaaaaaactctagttcaaaaaaaatgcatcttttctctatttg

SEQ ID NO: 17 - DNA, 1575 bp, Solanum lycopersicum
  source 1..1575: mol_type = genomic DNA
atggatacttgttgtgcggctatgaaatcgacggttcatttggggagagtgagcactggtggctttaacaatggagagaaggagatttttggggagaagatcagagggagtttgaacaacaatctcaggattaatcagttgtcgaaaagtttgaaacttgagaagaagattaaacctggggttgcttactctgtgatcactactgaaaatgacacagagactgtgttcgtagatatgccacgtcttgagagacgccgggcaaatcccaaggatgtggctgcagtcatattaggaggaggcgaagggaccaagttattcccacttacaagtagaactgcaacccctgctgttccggttggaggatgctacaggctgatagacatcccaatgagcaactgtatcaacagtgctattaacaagatttttgtgctgacacagtacaattctgctgccctgaatcgtcacattgctcgaacgtattttggcaatggtgtgagctttggagatggatttgtcgaggtactagctgcaactcagacacctggggaagcaggaaaaaaatggtttcaaggaacagcagatgctgtcagaaaatttatatgggtttttgaggacgctaagaacaagaatattgaaaatatccttgtattatctggggatcatctttataggatggattatatggagttggtgcagaaccatattgacagaaatgctgatattactctttcatgtgcaccagctgaggacagccgagcatcagattttgggctggtcaagattgacagcagaggcagagttgtccagtttgctgaaaaaccaaaaggttttgagcttaaagcaatgcaagtagatactactcttgttggattatctccacaagatgcgaagaaatccccttatattgcttcaatgggagtttatgtattcaagacagatgtattgttgaagctcttgaaatggagctatcccacttctaatgattttggctctgaaattataccagcagctattgatgattacaatgttcaagcatacattttcaaagactactgggaggacattggaacaattaaatctttctataatgctagcttggcgctcacacaagagtttccagagttccaattttacgatccaaaaacacctttttacacatctcctaggttccttccaccaaccaagatagacaattgcaagattaaggatgccataatttctcatggatgttttttgcgagattgcactgtggaacactccatagtgggtgaaagatcgcgcttagactgtggtgttgaactgaaggatactttcatgatgggagcagactactaccaaacagaatctgagattgcctccctgttagcagaggggaaagtaccgattggaattggggaaaatacaaaaataaggaaatgtatcattgacaagaacgcaaagataggaaagaatgtttcaatcattaacaaagatggtgttcaagaggcagaccgaccagaggaaggattctacatacgatcagggataatcattatatcagagaaagccacaattagagatggaacagttatatga

SEQ ID NO: 18 - protein, 524 aa, Solanum lycopersicum
MDTCCAAMKSTVHLGRVSTGGFNNGEKEIFGEKIRGSLNNNLRINQLSKSLKLEKKIKPGVAYSVITTENDTETVFVDMPRLERRRANPKDVAAVILGGGEGTKLFPLTSRTATPAVPVGGCYRLIDIPMSNCINSAINKIFVLTQYNSAALNRHIARTYFGNGVSFGDGFVEVLAATQTPGEAGKKWFQGTADAVRKFIWVFEDAKNKNIENILVLSGDHLYRMDYMELVQNHIDRNADITLSCAPAEDSRASDFGLVKIDSRGRVVQFAEKPKGFELKAMQVDTTLVGLSPQDAKKSPYIASMGVYVFKTDVLLKLLKWSYPTSNDFGSEIIPAAIDDYNVQAYIFKDYWEDIGTIKSFYNASLALTQEFPEFQFYDPKTPFYTSPRFLPPTKIDNCKIKDAIISHGCFLRDCTVEHSIVGERSRLDCGVELKDTFMMGADYYQTESEIASLLAEGKVPIGIGENTKIRKCIIDKNAKIGKNVSIINKDGVQEADRPEEGFYIRSGIIIISEKATIRDGTVI

SEQ ID NO: 19 - DNA, 5070 bp, Solanum melongena
  source 1..5070: mol_type = genomic DNA
acccacaagtacatgtatggtactttcgtaaattctcacaaaactaggtctatagagttatcattaatatttaatatcagtactataaaaccaaaacctcccattcaatcatattatcagtgccacacactccatattagagtgaacatttgataacaatggcgtcttccattggagctttaaaatcttcaccttcttcccataattgcatcattgagagaagaaatgattctacacgtgcaatatccgctagaaatctctcattttcgtcttctcatctcgccggagacaagttgatgcccctgtcccaaggactccgattcagtgggagaagagctccgttgatcgtgtcgcctaaggctgtttctgattcacaaaattcgcagacatgtcttgacccggatgcgagcaaggtaagcaaattttagctcaattttatcaggaaatttgggaataaagtatataaaccattatgtatggtgtgagtgtgtgaaatgtagagtttttattaaatttttttctccgattgattgctaaaatgagtcgattatcggatgtggaggttgggagaagtagatcctttacttgtggaggtgagagtatgaaaaggatggaatggattgcttcagtgtgaagtattttgttctagatccaagatcctctcacctcttccctacttataatcacgtgcatattaaaaaaatatgattaatcgacagtttatttgtttaatattacccaaaattcttttctaaaaacatatttcagcgtttcattatggacatgatagtcatatactgatgaaattttgatttggaaagtttttttaaaggtagcatgatcttctggaaactgtaataattggatccgactatattatcatgagcagggcatcacattcccgaatgtcggttgttgtagcgtctgcctgaccttctaatcctctgccatttcaaaatgtcaaaaaaaaaaaggacatagttttgcacagatattgccatgaaatagagtctaagtagatgtactatattgtaatctagttatattttgagtgaacaatattctaacttgttgtttcctcaagtggcaataactttacgtcttgttagaagtaaagtttgttttttgatatgaaagctttaagacactgagtttctgtattgttaatgggatttggtgtagagtgttttgggaattattcttggaggtggagctggaacccgactttatcctctaaccaagaaaagagcaaagccagcagttccacttggagcaaattatcgtttgattgacattcctgtaagcaattgcttgaacagtaacatatccaagatctatgttctcacacaattcaactctgcgtctttgaatcgccacctttcacgggcatacgctagcaacatgggtggatacaaaaacgagggctttgtggaagttcttgctgctcagcaaagtccagagaaccctgattggttccaggtaactcctcattcctgctgcacacaattcacataaagctactgttattgttattaattacctggagacagatctccctcaattactgtgaagcatacaatgtagcatttaaactgcctgtatcttcctgagttttcaaagagctgtttataaatgcattttatttgggatgcaattcaggtttcatttttgtatgagtcataacaccttgaattaaacttctatttgtgttttgttgtgctttgagctagtttacttcacaaactggcataacaagtgatgatacttctctgtagggcactgctgatgctgtcaggcagtatctgtggttgtttgaggagcataatgttcttgaataccttatacttgctggagatcatctgtatcgaatggattatgaaaagtttattcaagcccacagagaaacggatgctgatattaccgttgctgcattgccaatggacgaaaaacgtgccactgcatttggtctcatgaagattgacgaagaaggacgcattattgaatttgcagaaaaaccaaaaggagagcaactgcaagcaatgaaagtaggactgtcatagtcttaatgcttttcaattcagaactactcactaaagaatttataagcacgtgttaaatcatgttttgatgtatttcaggtggataccaccattttaggtcttgatgacaagagggccaaagagatgccttttatcgcaagtatgggtatatatgtcattagcaaagacgtgatgttaaacctacttcgtgacaagttccccggggccaatgattttggtagtgaagttattcctggtgcaacttcactcgggatgagagtacgtactttctctagaacttacacttcgtgttacttatcacaaaaattagaatatgaataaatatacacatttttacagtgctcaatggaaaaaaattgcatcttctgtcattcactctagagcaatttaatatttgtggccttattgtcttaacttcttgaactctcttccaaatatagttatgaaaatgagctctctttacttttgtgttttcgtctattgccctctcatgtattttctctatttttcttcttttctccttgttgcttttcttgttctgctcatcataaatgtggaaactcaaaaaggaatgacgcatcgtgctcactttcttttaagataatggtcatttgtagaggtcttatgtatccatgttctcaggtgcaagcttatttatatgatgggtactgggaagatattggtaccattgaagctttctacaatgccaatttgggcattacaaaaaagccggtgccagattttaggtaattccgctgatctcaagcagatagttctctgcagtgtgcgtgctctgttatctactcactttgattaagcactaatatggctgtcttcaatatttgctgaagcttttatgaccgatcagccccaatctacacccaacctcgatatctgccaccttcaaaaatgcttgatgctgatgtcacagatagtgtcattggtgaaggttgtgtgatcaaggtaacttattattgttttaaggcctcaatatttataagttacattgttgatagcaaacatgtgaggcatatattgacgtgttcaatgttttctgtgacctcatccataatccgtattctttacaatggaagtcccacctgcatagaatacaaaatgaagttgagactactgctttctgaggtgtgcttgcagttttttctcagtttaatttgctatcgtggttggagagggaaattttttaaatttttttcttctaccggagatttgagattgtgatactgagatggtactccctctattttaatgtaattgtcccaagtttcgtgctgtatgaagaatatgagtgaaaacaaatgacgatatatatcatagagagaattggtcaaaatatgatatctaaagttagctctttgataaagttgaggtagcattaaataaaaatcactgggtacctataggttgtaggttcgaatcatcaaccgcttattcttacatagggtagtctacctacattatacctctaggtttgacccttctctggaccttgcataaacatgggatgcttcatgcaacaccctttttctaataatggagaatgaaaaaatatttgtggtggacctaaaagggaaattaggacgatctgttttggacggatcaaggtagtggtttatagtaccttgtcttggtggagaaaaaattgaagtcttgtaggaagaaacatatattgaacctctaatgtaagagaattccaaattattgagttttgcaatgaaaaagtccagattgatcaaaatggatattgaggattcaaataactgcccagactagtttatgcttgggtcatagtagtttttgttttagggcgtcaaggaagggagtgggatgaaggtaattactgagacactatcaccaattcttttcgtttcattttccatcagactgtgctccacaaaatacagaaaaaggttataccagtacatagttgttaaactcaatatttctgtgtgcagaactgtaagattcaccattccgtggttgggctcagatcatgcatatcagagggagcaattatagaagacacacttttgatgggggcagattattatgaggtaatgctagtaattcttatgttaccaaagatatataataatgtaatataaacgggaacatgaatttaatgttcttcacttattagacttgctgtatgtgcagtctattgtggagcaagttgccttagagtcaaattacagaggataaagaaaataacgaactagtggtttccttactttgttcacttattctaacggactcactttctgacccattatttgttcagactgatgctgacaggaagtttctggctgcaaagggtagtgtcccaattggcatcggcaagaattctcacattaaaagagccattattgacaagaacgcccgcataggggacaatgtgaaggttaaattccttgaactttcatgctttaaacatgcagtagatgcaatggtttttctcaacatcatgtgttcagattcattacattgattctttcatttagtcatgtagaaatcttttcttcattatctggcggacatacagtgatcatgagttcctaatgagtgattttcttttgtcagatcattaacaaagacaacgttcaagaagctgctagggaaacggatggatacttcatcaagagtgggattgtcactgtcatcaaggatgctttgattccaagtggaatcatcatttgaaggaacttgttgagacttggttgtcctccaagattttggctaaacagccatgaggtagaaatctagtgtacatcttacttttatgttgctagatacttctcattacatctataaaagaagactggatgctgtataaatttttcatctaaaataaaaatctgctttgatctttgcttttgccttaatttacttgtgttctgtctctgaatgtttaataatgaaaagaaattatatgttcttattaatcaattttcctgctcgtcttttagtaatcaccaggttcccccgcagtttg

SEQ ID NO: 20 - DNA, 1554 bp, Solanum melongena
  source 1..1554: mol_type = genomic DNA
atggcgtcttccattggagctttaaaatcttcaccttcttcccataattgcatcattgagagaagaaatgattctacacgtgcaatatccgctagaaatctctcattttcgtcttctcatctcgccggagacaagttgatgcccctgtcccaaggactccgattcagtgggagaagagctccgttgatcgtgtcgcctaaggctgtttctgattcacaaaattcgcagacatgtcttgacccggatgcgagcaagagtgttttgggaattattcttggaggtggagctggaacccgactttatcctctaaccaagaaaagagcaaagccagcagttccacttggagcaaattatcgtttgattgacattcctgtaagcaattgcttgaacagtaacatatccaagatctatgttctcacacaattcaactctgcgtctttgaatcgccacctttcacgggcatacgctagcaacatgggtggatacaaaaacgagggctttgtggaagttcttgctgctcagcaaagtccagagaaccctgattggttccagggcactgctgatgctgtcaggcagtatctgtggttgtttgaggagcataatgttcttgaataccttatacttgctggagatcatctgtatcgaatggattatgaaaagtttattcaagcccacagagaaacggatgctgatattaccgttgctgcattgccaatggacgaaaaacgtgccactgcatttggtctcatgaagattgacgaagaaggacgcattattgaatttgcagaaaaaccaaaaggagagcaactgcaagcaatgaaagtggataccaccattttaggtcttgatgacaagagggccaaagagatgccttttatcgcaagtatgggtatatatgtcattagcaaagacgtgatgttaaacctacttcgtgacaagttccccggggccaatgattttggtagtgaagttattcctggtgcaacttcactcgggatgagagtgcaagcttatttatatgatgggtactgggaagatattggtaccattgaagctttctacaatgccaatttgggcattacaaaaaagccggtgccagattttagcttttatgaccgatcagccccaatctacacccaacctcgatatctgccaccttcaaaaatgcttgatgctgatgtcacagatagtgtcattggtgaaggttgtgtgatcaagaactgtaagattcaccattccgtggttgggctcagatcatgcatatcagagggagcaattatagaagacacacttttgatgggggcagattattatgagactgatgctgacaggaagtttctggctgcaaagggtagtgtcccaattggcatcggcaagaattctcacattaaaagagccattattgacaagaacgcccgcataggggacaatgtgaagatcattaacaaagacaacgttcaagaagctgctagggaaacggatggatacttcatcaagagtgggattgtcactgtcatcaaggatgctttgattccaagtggaatcatcatttga

SEQ ID NO: 21 - protein, 517 aa, Solanum melongena
MASSIGALKSSPSSHNCIIERRNDSTRAISARNLSFSSSHLAGDKLMPLSQGLRFSGRRAPLIVSPKAVSDSQNSQTCLDPDASKSVLGIILGGGAGTRLYPLTKKRAKPAVPLGANYRLIDIPVSNCLNSNISKIYVLTQFNSASLNRHLSRAYASNMGGYKNEGFVEVLAAQQSPENPDWFQGTADAVRQYLWLFEEHNVLEYLILAGDHLYRMDYEKFIQAHRETDADITVAALPMDEKRATAFGLMKIDEEGRIIEFAEKPKGEQLQAMKVDTTILGLDDKRAKEMPFIASMGIYVISKDVMLNLLRDKFPGANDFGSEVIPGATSLGMRVQAYLYDGYWEDIGTIEAFYNANLGITKKPVPDFSFYDRSAPIYTQPRYLPPSKMLDADVTDSVIGEGCVIKNCKIHHSVVGLRSCISEGAIIEDTLLMGADYYETDADRKFLAAKGSVPIGIGKNSHIKRAIIDKNARIGDNVKIINKDNVQEAARETDGYFIKSGIVTVIKDALIPSGIII

SEQ ID NO: 22 - DNA, 3153 bp, Solanum melongena
  source 1..3153: mol_type = genomic DNA
tagctatgttttcctatcaattcattgaaagaatgatgttatctgtcacagaatgctcccatctggaataatggcgcccttcagtattacaactatgccttcagtgataatgtaaccatttgcttcattattgcactcctggacattttccttattcaaaatctgcatgaaaaatgatatcacaatatgctgtggacattttcggcatccaacaccttatcctcactagtatctatacatgtttaaagaagattttgagcttgatcaaactaccttaacatttttgccaattctcacattcttatcaattattgctttctttatctgtgagccttctccgatgccaataggaatgtgcatatcatcctctgcgcgaccactctgcatattttcaccatgttaaatatcctgcccctagaaacttaatgcttcttattattgccatgatcgataagaatcaaatgtagagatacgagggatctttcaaaaaaaaaaatttacttgaatatgaactggattcatatagtctccatttcgtctatgtgaccatccatgaataccaaatgaaatgtattcatgtgtcttacttggtaagtatcagatcccattattactgaatcctcaataatcgctccgtctcctactcttgtcctcattccaaccacagtacctctgatattgcatctctgcactcggaaaacacaaaatatggaacaaagacgtttgtgagaccaaattcttgggaaaaattacataactttatctaaatttcaaggacaattaatcactttttatacattgagaaagcatccgtctccgatcacagagtctgttattgcagcatcagttatctgagttggaggcaggtgtcgaggcaaagtgtataacggagagcctttgtcatacaagctgctaacgaagatgaaaaaaagccgttaggtgatatataagggatttactcttgagacttattcgtttttggagatgctgacttatatgccttctttgtgctttccatatttgcttggtaataagcttcaatgcttctcatgtcctcccagtagccgctgaatttgtgtgcgtatacctgtttgttccaaagaatggttcatgaagagtgaaagacaaaatgttgaatatctttatggcaacttagagcagtaagttgctcctgtatggaaataaacgactcttacattcattcctaaggaaattgcacctggtacaacttctcttctgaagtcattggccatggggtgatactccgtaagaagctttatcatggcatctttgttaacagcatatatatccataccggcatacttttgttgagaaatgtctgaattctgcacctgaaaatgatataatgaggaacaattgaaagtacgcggagaaagaaaaggagaaactcactactctcatctccggattgtctttgaatgaaatgacttgactttctgaatttacttggaaagtaccaagagctatatcattattatccctctttctgcttaacactgcaacagttatatcggctttattgtcgcagtggacctttaagagttcctggaaatccattttgtaaagatggtaaccaggaagaaccagaaaatcaacaattggatactcttctagtacccacaaacatcttcttatagcatcagcagttccctgaaaacacaaagaagcataacagtccgatggtgagaagaaggtatgaaatcattcaaattctcattttactatgttcaactctacgtaggtaggaaaacacagttgttgttctttcttttacctgaaaccagcccttgccttcaggagtctgataagctgcaatgacatcaactaatgcttcgttcctgaagcgtgcaccggagtaagctctcgagaggtgagaattgagagaggcagagttaaattgtgttagtgcataaatcttgttgatattactattgatgcagttgctaataactgcatcaatgagcctgtagtttcctgctataggaagtgcagcttctgatcttttctttgtcaatggataaagtcttgaatttgatccatctccaaacactattgctgcaacattctgccaaacactagtaaacatgaacatctacactgacattttattgtttgtaaacaagcagtagaaaatactccctccgtccatttttacttgtctcctgtactattttgggatgtccaacaatacttgtccattttatgaaatcaagagataattttacacttagttaattatagtcatttctctattatatttttcaagacactgtatttattatattcaaaggttaatttggtaaaattactccatcgtttactatttcttaatccctgcaccaagtttatagtggacaagtaaagattgacggagggagtagtaacaagagatattggtccaggcccaactgtcaaggtattgctcactctcaaaagacgcctcggcagttgaattctgggcccgccccccttactttcccctagcttatttatgtcataagatataatccctcttcattgaatatttactctatttgtatgccataatcgaggtgtttgtggtcttcgtttttcaagaacacattactaataaggggtaaaatggacataaagaaagatattgacatttacttgtcaagtattgatttggcacactttttaaatagtaatagatagaatgacaattttattgaatataataaactcaatgttgaaaaatgcattgttaaatgactatagctaaagataaagataaattaaaaactaaatgataaattagatctcttggttatctaaactgaaccaataaaagtagacatttattttagtacaacaaacaagtaaaagcagacatgggaataagtgtaacaaacattatctcgaacttctaaaataacaagtatttcgagacatatatttttagcaaggacgacagacggagagagtaaaaaagttggagaaagtgtggatatataaggaaaatgtcatgtaaacctggtctattggaggacatagaatgtttggacattcagaaagatgtgtatatggtggattcgactttgatggaacagaagatggaatgagccttgagttaatcttgagatttaaccaagtatgaagagacatgactaa

SEQ ID NO: 23 - DNA, 1368 bp, Solanum melongena
  source 1..1368: mol_type = genomic DNA
atgtctcttcatacttggttaaatctcaagattaactcaaggctcattccatcttctgttccatcaaagtcgaatccaccatatacacatctttctgaatgtccaaacattctatgtcctccaatagaccagaatgttgcagcaatagtgtttggagatggatcaaattcaagactttatccattgacaaagaaaagatcagaagctgcacttcctatagcaggaaactacaggctcattgatgcagttattagcaactgcatcaatagtaatatcaacaagatttatgcactaacacaatttaactctgcctctctcaattctcacctctcgagagcttactccggtgcacgcttcaggaacgaagcattagttgatgtcattgcagcttatcagactcctgaaggcaagggctggtttcagggaactgctgatgctataagaagatgtttgtgggtactagaagagtatccaattgttgattttctggttcttcctggttaccatctttacaaaatggatttccaggaactcttaaaggtccactgcgacaataaagccgatataactgttgcagtgttaagcagaaagagggataataatgatatagctcttggtactttccaagtaaattcagaaagtcaagtcatttcattcaaagacaatccggagatgagagtagtgcagaattcagacatttctcaacaaaagtatgccggtatggatatatatgctgttaacaaagatgccatgataaagcttcttacggagtatcaccccatggccaatgacttcagaagagaagttgtaccaggtgcaatttccttaggaatgaatgtatacgcacacaaattcagcggctactgggaggacatgagaagcattgaagcttattaccaagcaaatatggaaagcacaaagaaggcatataacttgtatgacaaaggctctccgttatacactttgcctcgacacctgcctccaactcagataactgatgctgcaataacagactctgtgatcggagacggatgctttctcaatagatgcaatatcagaggtactgtggttggaatgaggacaagagtaggagacggagcgattattgaggattcagtaataatgggatctgatacttaccaaagtggtcgcgcagaggatgatatgcacattcctattggcatcggagaaggctcacagataaagaaagcaataattgataagaatgtgagaattggcaaaaatgttaagattttgaataaggaaaatgtccaggagtgcaataatgaagcaaatggttacattatcactgaaggcatagttgtaatactgaagggcgccattattccagatgggagcattctgtga

SEQ ID NO: 24 - protein, 455 aa, Solanum melongena
MSLHTWLNLKINSRLIPSSVPSKSNPPYTHLSECPNILCPPIDQNVAAIVFGDGSNSRLYPLTKKRSEAALPIAGNYRLIDAVISNCINSNINKIYALTQFNSASLNSHLSRAYSGARFRNEALVDVIAAYQTPEGKGWFQGTADAIRRCLWVLEEYPIVDFLVLPGYHLYKMDFQELLKVHCDNKADITVAVLSRKRDNNDIALGTFQVNSESQVISFKDNPEMRVVQNSDISQQKYAGMDIYAVNKDAMIKLLTEYHPMANDFRREVVPGAISLGMNVYAHKFSGYWEDMRSIEAYYQANMESTKKAYNLYDKGSPLYTLPRHLPPTQITDAAITDSVIGDGCFLNRCNIRGTVVGMRTRVGDGAIIEDSVIMGSDTYQSGRAEDDMHIPIGIGEGSQIKKAIIDKNVRIGKNVKILNKENVQECNNEANGYIITEGIVVILKGAIIPDGSIL

SEQ ID NO: 25 - DNA, 6024 bp, Solanum melongena
  source 1..6024: mol_type = genomic DNA
acacttattgtttcctacttcctagtcctctagttcacaacatcaacctccataatttacataaatacaaacaggccatttcacttctcttaattctttctcagagagaaaaatggctgttgctgccgacgggcgatttgccttacttcatagtaagcctgcaacattgacagggagagaaaacctcagggtaataaagttttgcaatggagaattgatgggaaagaaactcaagttcacaaagtttcagttgaggactaatgtagtgaagcctaatatctgcatgtcacttacagctgatgttgcaggtgaggctaaggtgagtgttcttacatttgcattttgtgcccccacctgcttgccatatatttctttgcaaatgagcactgggactacgatgggccacttcttaacatcaatagtaacaagttagatcaaatgatacatcctgaattaatttgatcacctcatgctaaaacaacttaaaattaaaatcttgaataccccattttgaccaaataataaatgtggacagttgaaggatcttgaaccgaagaaggaggatgcaaggacagtagtagcaatcattctaggaggcggagcaggcactcgtcttttccccctcaccaagcgtcgtgctaagcctgcagtaagttttcattcacattatcagagaaaatatcactacgagtttttactgagctaactgcatctcatcattgagtcaatatcaatttgtttcaggttccaatcggaggagcatataggctaattgatgtaccaatgagcaactgtattaatagtggcatcaacaaagtatacattctaactcaattcaactctgcctcacttaacagacatatctctcgtgcttacaacttcggcaatggggtcacattcggagatggctatgtcgaggtaaacataaaacatcaatcttcattttccagtatgcatatagtttcagggtttaatgatattgtagcatcatggctgacaagttgtagcacaggtcttagcagcaactcaaacaccaggtgaattaggtaaaagatggttccaaggtactgcagatgctgtgaggcaattccactggctttttgaggtacacacatgttcaggttgaattacgatatccatatttttccgcaagtatcaacatattcctgaactgcaatatttctgaagaacaaatcaaaatgtatatgttcatgaatttcaattcttaattgtatcagttagtttcttccagggaacttaatcatgaggaaaaatacagtacaacagcatgtaatataaacaatactttttctacattcagctatagctatcccaaaattatctcatcatatggtttagttacaaagatgacattaattctggtaaacattgagtttgaaatgttcaccctgattttccaaaatttaaggatgcaagaagcaaggacatagaagatgtgctcattctctctggagatcacttgtatagaatggattacctgaactttgttcaggtaaagactagactaagctatattggtatcatatatcttaatattacttattactccacattgatttcctcctgcttgacagagtcatcggcaaagtggtgcagacattaccatatcaagcttgccaatagatgacaggtagcattaggaggaataatctagactcaagatagactatgacttcctctcactgatggcatactttctcaaagactgctttgaaaatcactacaagagataatttattctattgaatcacttctatgtttcatgatagacgagcttcagattttggcttgatgaaaattgatgacacaggacgggtcctgtccttcagcgaaaagccaaaaggagatgatttgaaagcgatggtaattcttgaaaatttgacagtctcacagcttaatctactttaaattttaaagttgctgcaatcacttgacaacaagtacttcaggcagtagacacatctgttctgggattatccccagaagaggctaaagagaaaccttacattgcttcaatgggagtttatgtattcaagaaggatattctcctgaatcttttaaggtatgtaacgagtttcttcgatagtacccttaattttgagggtgtcatggtccacatgcaaatgagagaaacttcggaaagaggacaaaaaaaccaagaaactaaagcatccagctaatagggatagctacactcaagagaagcatattctggtgttcactttaaagcagaaagagactgcataaaatcttatgttagccctcagttccctcatactctgtctatagtgacatgctgaagagatcagacaaaagatcatgaaatgctaactctttctcttcttagatggcgtttcccgactgcaaatgactttggttcagagattatccctgcctctgccaaagaattctgtgtcaaggtgtgtaccacttaatttatagttggtagttatacaagacgttgatctgtttgttcaaaaagtttactacttcatccaatcttgttgtttccatcacaaggcaggatattctatgtacatatggtactacgttgcagttacagcctcacaatttgtctccccctctatcatcttatccccacccttacccccacctccacccctcccatgtcttcattgtcccaaaagaattcaaatcactatttcagttatggtttcctaagcagcttttcaacctgtatggtaactaaagcaaattttgcttgatgttgcaggcttgcttattcaatgattactgggaagatattggtacaatcagatccttttttgaagcaaaccttgcactcactgaacatgtaagaaagagagctattatatgttttttttttttagtctttaaagggttttacttcttcaaacaacctgaataaggtggaatctggctactcaaacctgtcaatcttatgtgacaatattaaaaaagattgattgtagtaataatatggccaactatgtttttccttttttcatttcagccaccaagatttagtttctatgatgcagcaaagccaatatacacgtcaaggagaaacttacctccatcagcgattgataatagcaaggttcaaggcacagacccagttcttcaatgttaaacagctatacatctttaaattttcttgcactaaccaaatctatttgttgccagattgttgattcaattgtatcacatggtagtttcttgaccaattgcttcgtggaacacagtgttgtcggcatccgatcccgtataggcaccaatgttcacttgaaggttagtctagtaacactgcatataatgctattgaaaattcagcattatgcttttgaacaacaaaaccaaaaattggttctccaactagactccacgtagatccacataagtacatttattgcttcatatttaaacaattagatgattccacacaaacacaaagcgtttacttgatttttgtaactgctgttaattctgcatgcacttggttgaaatccatacaggacacagtgatgcttggtgctgactactatgaaactgatgctgagattgcttcacagctagctgaaggaaaagtgcctttaggaataggagagaataccagaataaagtaaatcactaagatggttctccctttgcatttatcaagtgagaaaacgaatgtgattctaatttcatctctcaccttgtttcaaccaatatgcagagaatgcatcattgacaagaatgcaagaattggaaagaatgtagttattgccaactcagaggtatgatcctcccatactccgaacaacagcaggaggtcaaatttcatcttcttattactgggactcacttattatcctcggatacatcaatttgcagggtatccaagaagcagacagatcctcagaaggattttacatccgatcaggtatcacagtaatatcgaagaactcaacaatcccagatggaactgtgatatncccttacccccacctccacccctcccatgtcttcattgtcccaaaagaattcaaatcactatttcagttatggtttcctaagcagcttttcaacctgtatggtaactaaagcaaattttgcttgatgttgcaggcttgcttattcaatgattactgggaagatattggtacaatcagatccttttttgaagcaaaccttgcactcactgaacatgtaagaaagagagctattatatgttttttttttttagtctttaaagggttttacttcttcaaacaacctgaataaggtggaatctggctactcaaacctgtcaatcttatgtgacaatattaaaaaagattgattgtagtaataatatggccaactatgtttttccttttttcatttcagccaccaagatttagtttctatgatgcagcaaagccaatatacacgtcaaggagaaacttacctccatcagcgattgataatagcaaggttcaaggcacagacccagttcttcaatgttaaacagctatacatctttaaattttcttgcactaaccaaatctatttgttgccagattgttgattcaattgtatcacatggtagtttcttgaccaattgcttcgtggaacacagtgttgtcggcatccgatcccgtataggcaccaatgttcacttgaaggttagtctagtaacactgcatataatgctattgaaaattcagcattatgcttttgaacaacaaaaccaaaaattggttctccaactagactccacgtagatccacataagtacatttattgcttcatatttaaacaattagatgattccacacaaacacaaagcgtttacttgatttttgtaactgctgttaattctgcatgcacttggttgaaatccatacaggacacagtgatgcttggtgctgactactatgaaactgatgctgagattgcttcacagctagctgaaggaaaagtgcctttaggaataggagagaataccagaataaagtaaatcactaagatggttctccctttgcatttatcaagtgagaaaacgaatgtgattctaatttcatctctcaccttgtttcaaccaatatgcagagaatgcatcattgacaagaatgcaagaattggaaagaatgtagttattgccaactcagaggtatgatcctcccatactccgaacaacagcaggaggtcaaatttcatcttcttattactgggactcacttattatcctcggatacatcaatttgcagggtatccaagaagcagacagatcctcagaaggattttacatccgatcaggtatcacagtaatatcgaagaactcaacaatcccagatggaactgtgatatgacaagtgtgacaatgtctttttgtcttttctttcaagtagcattggcaccaagaaaattgtgaaatatgagaagtaaaagtttcagattatcaacttttcctcattcagcaagtaaatattgaactacaggctgatgataaaaagggttgtttctaccattgacgaattttaaaccaagaataaaatgtctgatcaaaggtgaacactctcagtacaaactagctgatgacactaccaaaatatggttttcaatatttataccagttggaaacaactttcttctaggaaggatacgaagtttaaacttacaggcaagactacttggtgagtcaataactgtacaacctttcactagattgctgcattattcaaatctttcctctgttttctcacatgtacctttgtctttttcgagaaaaacacatgtactgcattctcaccaacacatctctactggatcttcttttcagaacttgggtggtaattgacaatttagtaaagataaaggaacatagggttcaaattcttaaaagagcacttgaagagaaccatcaatgttaacattttaccctttttttcagcaaatgtgttgcttaaattcaaaaattacacagctgtagaacttatgaagagtcaaaggacaaatactctacgatgtaatattctgagcaaaattcatagtgtg

SEQ ID NO: 26 - DNA, 2085 bp, Solanum melongena
  source 1..2085: mol_type = genomic DNA
atggctgttgctgccgacgggcgatttgccttacttcatagtaagcctgcaacattgacagggagagaaaacctcagggtaataaagttttgcaatggagaattgatgggaaagaaactcaagttcacaaagtttcagttgaggactaatgtagtgaagcctaatatctgcatgtcacttacagctgatgttgcaggtgaggctaagttgaaggatcttgaaccgaagaaggaggatgcaaggacagtagtagcaatcattctaggaggcggagcaggcactcgtcttttccccctcaccaagcgtcgtgctaagcctgcagttccaatcggaggagcatataggctaattgatgtaccaatgagcaactgtattaatagtggcatcaacaaagtatacattctaactcaattcaactctgcctcacttaacagacatatctctcgtgcttacaacttcggcaatggggtcacattcggagatggctatgtcgaggtcttagcagcaactcaaacaccaggtgaattaggtaaaagatggttccaaggtactgcagatgctgtgaggcaattccactggctttttgaggatgcaagaagcaaggacatagaagatgtgctcattctctctggagatcacttgtatagaatggattacctgaactttgttcagagtcatcggcaaagtggtgcagacattaccatatcaagcttgccaatagatgacagacgagcttcagattttggcttgatgaaaattgatgacacaggacgggtcctgtccttcagcgaaaagccaaaaggagatgatttgaaagcgatggcagtagacacatctgttctgggattatccccagaagaggctaaagagaaaccttacattgcttcaatgggagtttatgtattcaagaaggatattctcctgaatcttttaagatggcgtttcccgactgcaaatgactttggttcagagattatccctgcctctgccaaagaattctgtgtcaaggcttgcttattcaatgattactgggaagatattggtacaatcagatccttttttgaagcaaaccttgcactcactgaacatccaccaagatttagtttctatgatgcagcaaagccaatatacacgtcaaggagaaacttacctccatcagcgattgataatagcaagattgttgattcaattgtatcacatggtagtttcttgaccaattgcttcgtggaacacagtgttgtcggcatccgatcccgtataggcaccaatgttcacttgaaggacacagtgatgcttggtgctgactactatgaaactgatgctgagattgcttcacagctagctgaaggaaaagtgcctttaggaataggagagaataccagaataaaagaatgcatcattgacaagaatgcaagaattggaaagaatgtagttattgccaactcagagggtatccaagaagcagacagatcctcagaaggattttacatccgatcaggtatcacagtaatatcgaagaactcaacaatcccagatggaactgcttgcttattcaatgattactgggaagatattggtacaatcagatccttttttgaagcaaaccttgcactcactgaacatccaccaagatttagtttctatgatgcagcaaagccaatatacacgtcaaggagaaacttacctccatcagcgattgataatagcaagattgttgattcaattgtatcacatggtagtttcttgaccaattgcttcgtggaacacagtgttgtcggcatccgatcccgtataggcaccaatgttcacttgaaggacacagtgatgcttggtgctgactactatgaaactgatgctgagattgcttcacagctagctgaaggaaaagtgcctttaggaataggagagaataccagaataaaagaatgcatcattgacaagaatgcaagaattggaaagaatgtagttattgccaactcagagggtatccaagaagcagacagatcctcagaaggattttacatccgatcaggtatcacagtaatatcgaagaactcaacaatcccagatggaactgtgatatga

SEQ ID NO: 27 - protein, 694 aa, Solanum melongena
MAVAADGRFALLHSKPATLTGRENLRVIKFCNGELMGKKLKFTKFQLRTNVVKPNICMSLTADVAGEAKLKDLEPKKEDARTVVAIILGGGAGTRLFPLTKRRAKPAVPIGGAYRLIDVPMSNCINSGINKVYILTQFNSASLNRHISRAYNFGNGVTFGDGYVEVLAATQTPGELGKRWFQGTADAVRQFHWLFEDARSKDIEDVLILSGDHLYRMDYLNFVQSHRQSGADITISSLPIDDRRASDFGLMKIDDTGRVLSFSEKPKGDDLKAMAVDTSVLGLSPEEAKEKPYIASMGVYVFKKDILLNLLRWRFPTANDFGSEIIPASAKEFCVKACLFNDYWEDIGTIRSFFEANLALTEHPPRFSFYDAAKPIYTSRRNLPPSAIDNSKIVDSIVSHGSFLTNCFVEHSVVGIRSRIGTNVHLKDTVMLGADYYETDAEIASQLAEGKVPLGIGENTRIKECIIDKNARIGKNVVIANSEGIQEADRSSEGFYIRSGITVISKNSTIPDGTACLFNDYWEDIGTIRSFFEANLALTEHPPRFSFYDAAKPIYTSRRNLPPSAIDNSKIVDSIVSHGSFLTNCFVEHSVVGIRSRIGTNVHLKDTVMLGADYYETDAEIASQLAEGKVPLGIGENTRIKECIIDKNARIGKNVVIANSEGIQEADRSSEGFYIRSGITVISKNSTIPDGTVI

SEQ ID NO: 28 - DNA, 5500 bp, Solanum melongena
  source 1..5500: mol_type = genomic DNA
tactctgctagttattcatcggtgacagctatgagatgatcttcagttagagatcctcaatagcagaaaccatcacctggcagtgaagaaatactcgagtgttcaagatttaacaaagtataaaaagtaatatctaacctatatacaatatcattttccagcaggtcctcaacataccacccttggtcatacatagcccctccacgccctctgcaaatgggggaaactgaagcgaggcagcaaaaacctctcatctagcagctgccagctgaaaacttaaagatgagggcctgagggtcacttagagatgtgtttcatcaagtccaagaaagtgaatatgtcatatgtggaaagtaaactacctcatgttctgttacgtacatgaaaagtgaccagtaaataacatgtcagaaaagtcctagtagcacattcatcatcactagaccataccagcctacttttatttcctttgaggatcaacttgattcactagcaagaacactttagcaagttttcatacgactgtcccatctttaatggttgcattcttcataatgacagtaatcccagacctaatgtaaaatccttctgtcggcctattagcttcttgaacaccctgcaacacccaagcatcagttaaacatgtattcacttttgtgctgatttcatacttcaaaagaaagatacgtacttccttgttcagaataaccacatcttttcctatcttcgcattcttgtcaattatgcaattgcttcaagaaaataggaagaaaagcatgtcaaaatcacagaagacaacctaatcgaagttcaggcaaactgtaaatacatccgaatgaataatgatacaataatcagtaggaccagagctcgaagttttcaagctggtcaatcaaaatggaagtctgggcagtcaattttcaatcaacaaggatgagaagtgagtacgagttcagtataaaatcttactgtatcttggtgtttggtccgacccctattggaacctttccttctgctagtagtgaagcgatctcagattcagtttgataataatctgcacccatcatcatggtatcctgagaagtgtaagacatattcagcctataaaaaagttagcactaaatagctgtttatgaagaaaggacctaaagtgctaagatcatgcttgatgttcattaccccactatgctaaagtccattcctagagtatatcataccagcttatttgcagaaaggaatgcaaactgtaaagcatatctggtgaaaaatatccagaactgtcataaatcatccttcctacaactagtttgacggcacactcacaggatggatgataaccattatgtgccttacatgtctgtcatctaggtttctaatatatcagaatgcatcagaggaatgcaggatattgtttttgatttagtccaagctctaactgatagaactctccagctaaaaacagataagaaaggctttcagtgaaaatatgaaaacataccttaaactcaacaccatagtctaaacgtgatcgaacaccaacaatagagtgctggatggagcattccctcagaaagcaaccatgggaaattattgcatccactatctgataatgaacagtattaatacatgttcatagcaacatgcaatcctggaaaatttttaccacaagtatttatgttaacttgcacaccctgcatttgtcgacttttgtaggaggtaagaacctagcagaagtgtagaatggggtcttcgggtcattgaagtcaaactttggaggctacatcaaacagggagaaaagagggaaaatgcaattcaataacaagttattaatcattaactgaaggtggagattatggtaaataaaaatgaaatataactaccaaattcgctgagatttagcattccacaattactaaaagtacagatccatatttaagagtccttgcatcaactgtcaagctcttactttgtgtaatagtagaactaagtcagatggtaggaaatattaagttgctcacctgctctgtgagggccaagttggcatcaaagaaagactttactgttccaatatcctcccagtaatcattaaacaaatatgcctaataagatcgagaagtcaaaacttaagtatacaatgaacagcaaagtttgtgaacttggaaaggctagaagttgcaattgagaaagttaccaccagctagtacctggacattgcaatccttcacagcagaagggatgatttcagagccaaagtcattgcatgaagggtatgctgacttgagaagttttagtaaaacatcagttttgaatacataaacacccatggatgcaatgtaaggaaaattggatgcttcttgctcagatagtccgagaattgatgtgccaacttgctgcaatagaatattagacatacaacacaagttatgtaggagaagagacgatgtgaaagcaagaggggctgtaaggggataactaatcaatagctaactccaatttagaatgaacttctacagttccaggtttggaagtccaaaaatatgtaccattgccttcaaagcagggccttttggtttctcagcaaattggatgatacgtcctgtttcatctattttcatcaatccaaaatctgaagcttgactgcaaaaagagaggatggggggaggtaagagagacaaaaaggtcttgattgactccctggtagagcttaaatgtctgctagcccttatagtgcattttagatagacaacatacctgaaatctgggagcagatacataagttagggatctcaaaaagcttcgtttttagaaatcgaaacagaagtagacttgaggggaactgcaaagtgcaaatctgtatgcttccagatcttttagtggatattaatcagtgctggtgctctgatgttaatgaggatgcaatatgggaattgtatcaacattattactatctatctgtgatggtggtgatagaaaagatactaggtccacttaaatggttaaaactcaaaaggatttcaagcttcctagttccaacaattttaattttcttttttgtatgctatttccttgtctgtgacaaatcgtattgaggattaatataaatcatgatgaagctgggcctttcacaattaaatctgtagatcgatatgtctgactaaaagtgccacttcaattttcaatatgccttcaacatattctgaacaactgtggccgccatttgccactctgaacaacactagaaagagcctttgaaagagttcagggagacagaaaacactatatttggtttcacatgaaataagctatcaagttagaagaaagatcaatctcatacccatcatccattggaacacatgaaactgtaatatcagcattcgtgtcaatatgcttctgcaagcacaaatattattcattaagcaaaatatatgaacataagattggggaggaagcaatgttgtcacaagttgctcatatacctgaacaaagtccatgtagtccattcgataaagatggtcaccagacaaaattaatatgtgctcgacattcttgttcttctgattctatgtcatcattcgaaaaaaaaggtcagcatttttcctagggagacttatcaatgaagatctcatttgcatacctcaaatacccatataaattgccgcacagcatcagcagtgccttggaaccacattttccctgaatcccctggagtctgggttgcagctaaaacctgcatttaaggaaaacttttgttttatctgtccagcacacatttcctttatcttccccatttttaggttgttcaacccaccactccatggattcacggatccagagcagaggaaatcaacaataaaaatatagatgactcgccaagcatctcataagaagtgtaatcttaaaaagatatgattaaacttcactgtcaaaatataagtagcttaaaagagcttagaataaaaacttcaagtaaccacaatgatcttacctccacaaatccatctccaaaacccactccatttccaaaattatatgtacgggcaaggtgacgattgagggaaaaggaattgaactgtgttaagatgaaaattttccgtatgccgctattaatgcagttactcatcggtacgtcaattagccggtaacaaccaccaattggaacctgaagaacaaatgcaatagtcacagaatttgaatgaaaatcgattgagataaacatctaagtacattcttaaagcttaattgaattgagaacagagaaggcaactctgcacttagcttaactactttctttagttcacaaagatttaagaatcactacaatttttaagactgaagttattatgaattggacatgttgtcaacctcaaaagacacatggtaccaagcgacaagagaagataaacatatatgtgtgtgtgtgtgtgcagtaattcttacagctggtttagctcttctgctggtaagaggaaaaagacgagttccagcaccaccacctagaatgacagaggcaacagattttggatctgcctttggatgctcctcaaacactgattcgtcaaatgactgaaaccaatatacataaataacacaaattaatcttcttaaatggatcattaactttacttcatcactcacctaattactctacttaccagcatctctttgtttatatcacgggtgagaatagaggagacaaccacacccttaacttttctacccctttcttcacttctgaaatggaccacaaaacccttgttcatcagtgttcttctactaatcttctgtccccaaaatgcactctcttggttcctaattgccactgattgggtagtgcccttcatattagcacacaacacatccattgctgcactactcactccagtggttaatcagccacgaaatacagaatcccagcaactacatcacataaagaagcacgttaattcgatttttcctaaaagattcctactcgagacagaaactgctaaaaacaatttacaaaaaggtgaccactttatccaaaaatatcaatacatagagaataaataaaatgcaaggccatgatcatcaatttaaataaaacccacatcacaaaaacaaatccttagcaaattaaataattttcattacccaaaaccccttttttttattaaactagatccgaaaacactcagatcatacaacttttttgcagaatccatactataatactatattattttttcaaagaaaaaaatgaactttctgactacctgatgtaagctttttcaaacacccaacttttcctcaaaagttttctgcagaaagtaattagaatgagttacaaattattaggttagagtttgttgataatccaatggattctaaagtctgtcgactctcttgagatttgcacggtccaacgtactataaacgtgaagctaacatttttaactctccttgagttactaaaaagcaggagaaagggaaatattacgggaattttcaaatatttcggca

SEQ ID NO: 29 - DNA, 1680 bp, Solanum melongena
  source 1..1680: mol_type = genomic DNA
atggatgtgttgtgtgctaatatgaagggcactacccaatcagtggcaattaggaaccaagagagtgcattttggggacagaagattagtagaagaacactgatgaacaagggttttgtggtccatttcagaagtgaagaaaggggtagaaaagttaagggtgtggttgtctcctctattctcacccgtgatataaacaaagagatgctgtcatttgacgaatcagtgtttgaggagcatccaaaggcagatccaaaatctgttgcctctgtcattctaggtggtggtgctggaactcgtctttttcctcttaccagcagaagagctaaaccagctgttccaattggtggttgttaccggctaattgacgtaccgatgagtaactgcattaatagcggcatacggaaaattttcatcttaacacagttcaattccttttccctcaatcgtcaccttgcccgtacatataattttggaaatggagtgggttttggagatggatttgtggaggttttagctgcaacccagactccaggggattcagggaaaatgtggttccaaggcactgctgatgctgtgcggcaatttatatgggtatttgagaatcagaagaacaagaatgtcgagcacatattaattttgtctggtgaccatctttatcgaatggactacatggactttgttcagaagcatattgacacgaatgctgatattacagtttcatgtgttccaatggatgatgggtatgttgtctatctaaaatgcactataagggctagcagacatttaagctctaccagggagtcaatcaagacctttttgtctctcttacctccccccatcctctctttttgcagtcaagcttcagattttggattgatgaaaatagatgaaacaggacgtatcatccaatttgctgagaaaccaaaaggccctgctttgaaggcaatgcaagttggcacatcaattctcggactatctgagcaagaagcatccaattttccttacattgcatccatgggtgtttatgtattcaaaactgatgttttactaaaacttctcaagtcagcatacccttcatgcaatgactttggctctgaaatcatcccttctgctgtgaaggattgcaatgtccaggcatatttgtttaatgattactgggaggatattggaacagtaaagtctttctttgatgccaacttggccctcacagagcagcctccaaagtttgacttcaatgacccgaagaccccattctacacttctgctaggttcttacctcctacaaaagtcgacaaatgcaggatagtggatgcaataatttcccatggttgctttctgagggaatgctccatccagcactctattgttggtgttcgatcacgtttagactatggtgttgagtttaaggataccatgatgatgggtgcagattattatcaaactgaatctgagatcgcttcactactagcagaaggaaaggttccaataggggtcggaccaaacaccaagatacacaattgcataattgacaagaatgcgaagataggaaaagatgtggttattctgaacaaggaaggtgttcaagaagctaataggccgacagaaggattttacattaggtctgggattactgtcattatgaagaatgcaaccattaaagatgggacagtcgtatga

SEQ ID NO: 30 - protein, 559 aa, Solanum melongena
MDVLCANMKGTTQSVAIRNQESAFWGQKISRRTLMNKGFVVHFRSEERGRKVKGVVVSSILTRDINKEMLSFDESVFEEHPKADPKSVASVILGGGAGTRLFPLTSRRAKPAVPIGGCYRLIDVPMSNCINSGIRKIFILTQFNSFSLNRHLARTYNFGNGVGFGDGFVEVLAATQTPGDSGKMWFQGTADAVRQFIWVFENQKNKNVEHILILSGDHLYRMDYMDFVQKHIDTNADITVSCVPMDDGYVVYLKCTIRASRHLSSTRESIKTFLSLLPPPILSFCSQASDFGLMKIDETGRIIQFAEKPKGPALKAMQVGTSILGLSEQEASNFPYIASMGVYVFKTDVLLKLLKSAYPSCNDFGSEIIPSAVKDCNVQAYLFNDYWEDIGTVKSFFDANLALTEQPPKFDFNDPKTPFYTSARFLPPTKVDKCRIVDAIISHGCFLRECSIQHSIVGVRSRLDYGVEFKDTMMMGADYYQTESEIASLLAEGKVPIGVGPNTKIHNCIIDKNAKIGKDVVILNKEGVQEANRPTEGFYIRSGITVIMKNATIKDGTVV

SEQ ID NO: 31 - DNA, 4876 bp, Solanum melongena
  source 1..4876: mol_type = genomic DNA
caaggtttctcgtgagagttcacaagccaataaagctgatcacacttgcccttgtccacttgatcactcaagaaacaccttcttgtgattcactttgaattctagatcttcttaattacctttcaagattcttgattttacaccactgagaatactaggaatttccattttctttgacattcttctcctaaaccttccaagatccaagatcatcactttccaggttcaaaatcttgtttcttcttcttataacaatgttacatgaaatctaaaatttctgaaacatgggtgaaccctttttgtaatatagtatgagtgtcagcacataatattatattatacctaatttattgttaaagagatcactggtttacatgtcgcatattttgggctacaaattctaatggttctaatggaggatattgtaaatattcatgttatggtaatgctatgagttactagtactaagcatatagctggcttggtttttgggtcactgaccatttttttaaattagtgatattttctttatgtattttatacttttcttggttgcttagagattacctgtactttattgagatttgagtacatctatttgatttggatccattgataagtcataatcttatgggattgctgatttcttgattttatgcagaaatggattcttgctgtgtggctatgaaatccactgctcatttggggggagtgaccactggtggctttaacaatggagagaaggagttttttggggagaagatcagagggagtttaaaaaataatttcaggattaatcagttgtcgaaaagtttgaaacttgagaagaaggagaacaagatcaaacctggggttgcttactctgtgatcactactgaaaatgacagagagactctggtaataattaatcctctctcttcattacgataaacaacatctctatctttaattttaatgaggtagtggtaaggtcttgtagttgttgttgttgttgatcttcattatgatatgtgtgcttgatacggtggaaacaagttgatatgaactatatgcaagtccaatattaaaatttctagtgttgaactcactacacttataatttgcgtacagtctgccttcttcagaccccacttgtgcgatttcactgggtatgttgtagttgttgatcttcattatgatgtgtgcttgatatggtggaaacaagttgatatgaactatatgcaaatccaatattaagatttctagtgttgaactcactacacttataagattataggttgggagtttaattttttgaagttaatactagtcttttgttaaatactcctttgttccatgttgaagatactgtgttcagttatagtatttatctccatcagcctgtgattgtgatttgtttgttggacctatttagattgtagatatgccgcgttttgagagacaccgggcaaatcccaaggatgtggctgcagtcatactaggaggaggtgaaggggccaagttattcccacttacaagtagaactgcaacccctgctgtaagagatttatctttgctcatgaactttacgaactgcgaacaaattatttgtgttaaatttctaccttaaacagatttacgagaaatagacagagaacaggtgtacaaaattgcaagttgatattcattctgtggtcgatttctaaagattctgatgttttgtgcaacatgacaccaggttcctattggaggatgctacaagctaatagacatcccaatgagcaactgtatcaacagtgctattaacaagatttttgtgctgacacagtacaattctgcccccctgaatcgccacattgcccgaacatattttggcaatggtgtgagctttggagatggatttgttgaggtttgagactatacttgtgataatcttgatgtaaactggattagtcttgctagatatcacagctgagctaattaatatgattaaatttgctagtcctccaagtgatttgttcacacaaaatctgtaacaatttcggatgatttcttcattgtttttctgctttctttcttcgttttacttactgaaaggaattcggtcataaaacagatactagctgcaactcagacacctggggaaacaggaaaaagatggtttcaaggaactgcagatgctgtcagacaattcatatgggtttttgaggttcgtctctataactttgtaccaccgctcttctgttgacctgatataatttcggaaaaatgtttcagtattttctcctgaatcagttattatgtacagatatatcgtacatttgtgatattaaggcaaccatttcttgtaggatgctaagaacaagaatgttgaaaacatccttatattatctggggatcatctctacaggatggattatatggagttggtgcaggtatgtccacttttaagtctttaactacctatgaactcctcattctatgtttgctttttatttttatcttaagcaaattttctgactgcctttgatcttcatacagaaccatattgaccggaatgctgatattactctttcgtgtgcaccagctgaggacaggtttgtttaaagtctaatgaattgatattgaatcattactcaagaaacaactagaaatggttctttcatgttaagagaattgagtgatctcagaaataagatggagaaagaatacttctcctttgatgttcagctgagattttgaaataaaaacatcatgttttctttattctttattgtaattgtgttaagctgagatttaagctttacattccagtcgagcatcagattttgggctcgtcaaaattgacagcagaggcagagttgtccagttttctgaaaaaccaaaagggtttgatcttaaagcaatggtaggtttacttgaactctttctttcattatatgtgctgatttctttccaaacaaatttcccttgttgttcagctgatgtcttctcgatctgcctactttctgtagcaaattctgctgcaactttttactacatataacaacatttggactagtgctcctttataattcaactcatatgtttttcattctgcagcaagttgatactactctcattggattatctccaaaagatgcgaagaaatccccctatattgcttcaatgggagtttatgtattcaagacagatgtattgttgaagctcttgaaatggagatatcctacagctaatgattttggatctgaaattataccagcagctatagacgaatacaatgtccaagtaaaggatcttggatctttttattgaaatatttttatgaatttagtacattagttgttagctgcagttttaacgaatttatgctgtcttctcaggcatacatattccgagactactgggaggacattgggacacttaaaactttttatgatgctagcttggcgctcacagcagaggcatgttgcaaattaccatttatgcttctcatcatatgcaatgaacttattttaactagctcattgcataagccaactgattaatttttcactgcagactgcaaagttccaattttacgatccaaaaacacctttctatacatctcctagtttccttccaccaaccaagatagacaactgcaaggtaagtgggcatattgtgcaagtgttttatattaactgttcttttatgggttctaacaggttttgttttctgactttcagattaaggatgccataatctcccatggatgtttcttgcgagattgttctgtggaacactccatagtaggtgaaagatcgcgcttagattgtggtgttgaactgaaggtttgcattttttatcttcctttgaagatttatcctcaacaagcctatttaattttccttagtttgtgaaaaaaattgcaccagttgattgattttagtacattttcttgtaaagtatggatctcaaacacatgttttactaccataccaggatacttatatgatgggagcagattattaccagacagaatctgagattgcctctctgttagcagaggggaaagtaccaattggaattggggaaaatacaaaaataaggtaagtggacattgaatttaacagtgaattcttttctgaagaatgtatttattgacacttgaggattcttaagtttgatctatggtttccctcatacaggaaatgtatcatcgacaagaacgcaaagataggaaagaatgttgtgatcatgaataaagatgtaagctttttttcctggtgccaactagttggtatattgagtcatgattgattgatcaagagagatcattggaactaactctcactgatgttcttttctttgaaactttaggatgtccaagaagcagcccggccagaggaaggattctacatacgatcagggataaccattatattagagaaagctaccattagagatggaacggtcatatgaactcgggaagcatctcttgttgaactactggaaatcaaaatctcaatgtgaggaaggtcaagggtgacctatttcccaacaaaaaccaagcacattcagcagctaactccctgaatgaagctttcttcgctcatgtaaaaagagcaagtataattgagacaccgcgtcttaaaataaaactggagctgctcctctagatgcctagttcatgttctaagggtcaaacgtgtgtaagatagcattctgaagaacacaacggtttatttattactcaacctcaacttttcgtattgtaaaaaactctacgttaaagaaatctcttatgataaatctcttattatgcactttttctctaagcattccaaatgctaatctcttatgatatctatatagaactatttgtgcctattctctctttaaagtttttatttta

SEQ ID NO: 32 - DNA, 1626 bp, Solanum melongena
  source 1..1626: mol_type = genomic DNA
atggattcttgctgtgtggctatgaaatccactgctcatttggggggagtgaccactggtggctttaacaatggagagaaggagttttttggggagaagatcagagggagtttaaaaaataatttcaggattaatcagttgtcgaaaagtttgaaacttgagaagaaggagaacaagatcaaacctggggttgcttactctgtgatcactactgaaaatgacagagagactctgattgtagatatgccgcgttttgagagacaccgggcaaatcccaaggatgtggctgcagtcatactaggaggaggtgaaggggccaagttattcccacttacaagtagaactgcaacccctgctgttcctattggaggatgctacaagctaatagacatcccaatgagcaactgtatcaacagtgctattaacaagatttttgtgctgacacagtacaattctgcccccctgaatcgccacattgcccgaacatattttggcaatggtgtgagctttggagatggatttgttgagatactagctgcaactcagacacctggggaaacaggaaaaagatggtttcaaggaactgcagatgctgtcagacaattcatatgggtttttgaggatgctaagaacaagaatgttgaaaacatccttatattatctggggatcatctctacaggatggattatatggagttggtgcagaaccatattgaccggaatgctgatattactctttcgtgtgcaccagctgaggacagtcgagcatcagattttgggctcgtcaaaattgacagcagaggcagagttgtccagttttctgaaaaaccaaaagggtttgatcttaaagcaatgcaagttgatactactctcattggattatctccaaaagatgcgaagaaatccccctatattgcttcaatgggagtttatgtattcaagacagatgtattgttgaagctcttgaaatggagatatcctacagctaatgattttggatctgaaattataccagcagctatagacgaatacaatgtccaagcatacatattccgagactactgggaggacattgggacacttaaaactttttatgatgctagcttggcgctcacagcagaggcatctcattgcataagccaactgattaatttttcactgcagactgcaaagttccaattttacgatccaaaaacacctttctatacatctcctagtttccttccaccaaccaagatagacaactgcaagattaaggatgccataatctcccatggatgtttcttgcgagattgttctgtggaacactccatagtaggtgaaagatcgcgcttagattgtggtgttgaactgaaggatacttatatgatgggagcagattattaccagacagaatctgagattgcctctctgttagcagaggggaaagtaccaattggaattggggaaaatacaaaaataaggaaatgtatcatcgacaagaacgcaaagataggaaagaatgttgtgatcatgaataaagatgatgtccaagaagcagcccggccagaggaaggattctacatacgatcagggataaccattatattagagaaagctaccattagagatggaacggtcatatga

SEQ ID NO: 33 - protein, 541 aa, Solanum melongena
MDSCCVAMKSTAHLGGVTTGGFNNGEKEFFGEKIRGSLKNNFRINQLSKSLKLEKKENKIKPGVAYSVITTENDRETLIVDMPRFERHRANPKDVAAVILGGGEGAKLFPLTSRTATPAVPIGGCYKLIDIPMSNCINSAINKIFVLTQYNSAPLNRHIARTYFGNGVSFGDGFVEILAATQTPGETGKRWFQGTADAVRQFIWVFEDAKNKNVENILILSGDHLYRMDYMELVQNHIDRNADITLSCAPAEDSRASDFGLVKIDSRGRVVQFSEKPKGFDLKAMQVDTTLIGLSPKDAKKSPYIASMGVYVFKTDVLLKLLKWRYPTANDFGSEIIPAAIDEYNVQAYIFRDYWEDIGTLKTFYDASLALTAEASHCISQLINFSLQTAKFQFYDPKTPFYTSPSFLPPTKIDNCKIKDAIISHGCFLRDCSVEHSIVGERSRLDCGVELKDTYMMGADYYQTESEIASLLAEGKVPIGIGENTKIRKCIIDKNAKIGKNVVIMNKDDVQEAARPEEGFYIRSGITIILEKATIRDGTVI

SEQ ID NO: 34 - DNA, 5030 bp, Capsicum annuum
  source 1..5030: mol_type = genomic DNA
cgttgagcacaagtccattaacaatggcagcttccattggagcattaaaatcttcaccttcgtccacacacaactgcattagaaatgagttcgcgtgtgcagtaccttctagaaacctctcgttttcgtcttctcatctctccggagacaagttgatgatgcctgtagcctcacaaggactccgattttgcgagagaagaactccagtcatcgtttctcctaaggctgtttctgattcgcaaaattcgcagacatgtctcgaccctgatgctagcagagtaagcaaaaaactacttattttgactcaattttatcactaactttgtgaatctataactatatacggtgtgagtgtgtgaaaatgtagaaaattttggtgagtttttgctctgattgatagctaacttggaggtgattatcggattgtggcggtttaggagaagcggatcctcttgtgaaaaaggatggagtggattgcttcagtttgaagtattttgactgtgtatagttctagatccgagatcctctcacctctttcccgcacataatcacgtgcttattttaaaaaatgtactaatgactacttttatttgtttaatattatgcaaaattctttgctatcattcttcatagattcattatgcacatgatagtcatacattgattaaattttttgactttagaaaaaaactgttagaactagtattatattctgcaaaatgtaatcattggatgatttattcccatctgtcctaccatctcctagccttggtatacagagttaccctgtatctgttgctggtgggaggtgaaatgtggtcatgaaaaataaatgtagtcatcggatctgactatcttagcataagcaaggaattgcaatcccgaataacgctttcaggcactgttggtagctaagttgcgctgactcttcactttcgatgccgcacctgattctccaaaaaaacactagctttggcgaatccgacacgcacacatttttgaagagtccgagcaacgtaggttagtagtgtctgtctcatcttctaatccttaaaccgtgcactttttatttgtaattagtaattatgggcagattgccatttcaatatgtcaactttgttataaaaaagtatattaatatagttatattgtgatttatggatgatcagtattgacttgttgtttccctgccaataactttgcaatgattatgtgtctgtttttcgatatgaaagcattaagaaaagcttacgttctatattgttaatgcaatttggtgtagagtgttttgggaattattcttggaggtggagctggaaccagactttatcctctaactaaaaaaagagcaaagccagcagttccacttggagcaaattatcgtctgattgacattcccgtaagcaattgcttgaacagtaacatatctaagatctatgttctcacgcaattcaactccgcgtctttgaatcgccacctttcacgggcatatgctagcaacatgggcggatacaaaaatgagggctttgtggaagttcttgctgctcaacaaagtccggagaaccctgattggttccaggtaagtcttcattcctgttgcacactattcacataaagctactgttattgttattaattaccgggagaatgatctcactcattactgtggaggatacaatgtagcacttaaactacctatatcttcctgagcttccgaagagctttttagaaatgcattatttgagatgtaactcgggtatcattcgtgcatgacaccttgaattaaacttgtatttgcagtttactgttactttgagctactttactttacaaactggcataacaaatgatgatacttgtttgtagggcactgctgatgctgtcaggcagtatctgtggttgtttgaggagcataatgttcttgaataccttatacttgctggagatcatctgtatcgaatggattatgaaaagtttattcaagcccacagagaaacagatgctgatattaccgttgctgcactgccaatggacgaaaagcgtgccactgcattcggtctcatgaagattgacgaagaaggacgcattattgaatttgcagagaaaccaaaaggagagcaactgcaagcaatgaaagtaagaccatcatagtcctaatgcttttcaattcagaactattcacaaaagaatttataagcatgtgttaactcgtgttttaatgtatgtcaggtggatactaccattttaggtcttgatgacaagagagctaaagaaatgcctttcatagcgagtatgggtatatatgtcattagcaaagacgtgatgttaaatctacttcgtgacaagttccctggggccaatgattttggtagtgaagttattcctggtgcaacttcacttgggatgagagtatgtactttcttctgtagaacttacacttagcgtttacttttatttatcgcaaaaaaatagaatatgtattatatactttttgcagtgttcaatgcataaaaagaattgcatcttctgtcatttagcatataaaactatactctagagcaatttagtatttgtggcccttattgtctcaacttcttaaacggtttccgactatggttatgaaaatgagctctcgttactcttttgctttcgtgtgttgcccctctcaagtttttctctgttttccttctcttctctttcttgcttctcttgttctgctcatcatcagtatgaatactcaaaaaagaatgatgcatcgtgctcaatttcttttaagaaaattgtgagttgtgacttggttagtgttatttctagaggtcttatgtatctgtgtttgcaggtgcaagcctatttatatgatgggtactgggaagatattggtaccattgaagctttctacaatgccaatttgggcattacaaaaaaaccagtgccagattttaggtaattctgctgatctctaagagataattctctgcagtttgcctgctttgctatctcactcactttgattaagcactaatatggctatcttcaatatttactgaagcttttacgaccgatcagccccaatctacacccaacctcgatatctgccaccttcaaaaatgctggatgctgatgtcacagatagtgttattggtgaaggttgtgtgatcaaggtaactaattattgtttcaaggcctcgatatttctgaattgcattcttaatagcaaacatgtgaggtgtatcttgacgtgttctatgtttttcatgacctcattcataatccatgttctttacaattgaagtcctacttgcatagaatacaaaataaagttgagactactgctttcttaaaaaaacaaaaggagataaatttaaggatactgaattctcgatttccctcctctcggagatgtgcttgcattttcttcttagtttaatttgctatcaggttggagagggaaaattattataaatattatagatcttgacttaaagatattttcctttacccggagattttgagattttgataccaagatggcactccctctattttagtgtaattgtcttagtttcgtgcagtatgaaatataggagtgaaagtaaataatgatatattatagagaaaattggtccaaaatatgacttctaaagttgactctttgataaagttgagatctcacaatattaaagtcattgactaatggagaaatgaaggggagccttggagcaacgtctctttgtagctataagtcacgggttcgagccattaactattaatgcttatgtagatacccctgggtttggcccttctctggaccccgcgtgaacgcaaggtgcttcgtgcaccgccctgctctttttctaaataatggagaatgaacaacatacttatgacggacctaaagggcaaattaagacatcctttttggacggaagaaggtagtgttagagtttatactaccttgtctcggtggagaaaaaattagtcttctaggaagaaacatatagatcattccagaatattgaattttaatgaaacagatccagattgatcgaaatggatattgagattcatatgactgaaccaactagcttatgattgaggtatagtagtagtttttgtttaggaccccgaggaagggattaggatgaaggtaattactgagacatctgtcaccaactttgtccttttcatttttcatcagactgtgctccagctctcagaactggcataccagtacatagttattaatcaatatttctgtgttgcagaactgtaagattcaccattccgtggttgggctcagatcatgcatatcagagggagcgattatagaagacacacttttgatgggggcagattattatgaggtaatccttaggttatcacaaaagatatctaatatcatatacacgggaacgtgaatttagctttcttcacttattatatttgcggtgtataacgtggtctactgtggagcaagttgccttggagtcaaattacagaggataaacaaaatattggacttgtgctttcgttactgcattcgcttagtattccgcactcactttctgtccctttattggtccagactgatgctgacaggaagttactggcttcaaagggcggtgtcccaattggtattggcaagaactctcacgttaaaagagccattattgacaagaatgcccgtataggggacaatgtgaaggttaaattccttgaactttcacgcttctgacttattgtcatttaaacagtcagtagatgtgtaacgttttttcttgacatcacatgttcagattcgtagcattgatttagtcatacagtggtcatgggtttctaatgagtaattttctttcatcagatcattaacagagacaatgttcaagaagcggctagggaaacggatggatacttcatcaagagtgggattgtcactatcatcaaggatgctttgattccaagtggtatcgtcatctgaaggaacacgttgaaacttggttgtcctccaaaattttggctaaacacatcca

SEQ ID NO: 35 - DNA, 1554 bp, Capsicum annuum
  source 1..1554: mol_type = genomic DNA
atggcagcttccattggagcattaaaatcttcaccttcgtccacacacaactgcattagaaatgagttcgcgtgtgcagtaccttctagaaacctctcgttttcgtcttctcatctctccggagacaagttgatgatgcctgtagcctcacaaggactccgattttgcgagagaagaactccagtcatcgtttctcctaaggctgtttctgattcgcaaaattcgcagacatgtctcgaccctgatgctagcagaagtgttttgggaattattcttggaggtggagctggaaccagactttatcctctaactaaaaaaagagcaaagccagcagttccacttggagcaaattatcgtctgattgacattcccgtaagcaattgcttgaacagtaacatatctaagatctatgttctcacgcaattcaactccgcgtctttgaatcgccacctttcacgggcatatgctagcaacatgggcggatacaaaaatgagggctttgtggaagttcttgctgctcaacaaagtccggagaaccctgattggttccagggcactgctgatgctgtcaggcagtatctgtggttgtttgaggagcataatgttcttgaataccttatacttgctggagatcatctgtatcgaatggattatgaaaagtttattcaagcccacagagaaacagatgctgatattaccgttgctgcactgccaatggacgaaaagcgtgccactgcattcggtctcatgaagattgacgaagaaggacgcattattgaatttgcagagaaaccaaaaggagagcaactgcaagcaatgaaagtggatactaccattttaggtcttgatgacaagagagctaaagaaatgcctttcatagcgagtatgggtatatatgtcattagcaaagacgtgatgttaaatctacttcgtgacaagttccctggggccaatgattttggtagtgaagttattcctggtgcaacttcacttgggatgagagtgcaagcctatttatatgatgggtactgggaagatattggtaccattgaagctttctacaatgccaatttgggcattacaaaaaaaccagtgccagattttagcttttacgaccgatcagccccaatctacacccaacctcgatatctgccaccttcaaaaatgctggatgctgatgtcacagatagtgttattggtgaaggttgtgtgatcaagaactgtaagattcaccattccgtggttgggctcagatcatgcatatcagagggagcgattatagaagacacacttttgatgggggcagattattatgagactgatgctgacaggaagttactggcttcaaagggcggtgtcccaattggtattggcaagaactctcacgttaaaagagccattattgacaagaatgcccgtataggggacaatgtgaagatcattaacagagacaatgttcaagaagcggctagggaaacggatggatacttcatcaagagtgggattgtcactatcatcaaggatgctttgattccaagtggtatcgtcatctga

SEQ ID NO: 36 - protein, 517 aa, Capsicum annuum
MAASIGALKSSPSSTHNCIRNEFACAVPSRNLSFSSSHLSGDKLMMPVASQGLRFCERRTPVIVSPKAVSDSQNSQTCLDPDASRSVLGIILGGGAGTRLYPLTKKRAKPAVPLGANYRLIDIPVSNCLNSNISKIYVLTQFNSASLNRHLSRAYASNMGGYKNEGFVEVLAAQQSPENPDWFQGTADAVRQYLWLFEEHNVLEYLILAGDHLYRMDYEKFIQAHRETDADITVAALPMDEKRATAFGLMKIDEEGRIIEFAEKPKGEQLQAMKVDTTILGLDDKRAKEMPFIASMGIYVISKDVMLNLLRDKFPGANDFGSEVIPGATSLGMRVQAYLYDGYWEDIGTIEAFYNANLGITKKPVPDFSFYDRSAPIYTQPRYLPPSKMLDADVTDSVIGEGCVIKNCKIHHSVVGLRSCISEGAIIEDTLLMGADYYETDADRKLLASKGGVPIGIGKNSHVKRAIIDKNARIGDNVKIINRDNVQEAARETDGYFIKSGIVTIIKDALIPSGIVI

SEQ ID NO: 37 - DNA, 1600 bp, Capsicum annuum
  source 1..1600: mol_type = genomic DNA
ttttgaaagatctttactattgttgccagaatgttgcagcatttgtgtttggagatggatcaaattcaagactttatccattgacaaagagaagatcagaatctgcacttcctatagcaggaaactacaggctcattgatgcagttgtcagcaactgcatcaatagtaatatcaagaagatttatgcacttacacaatttaactctgcctctctgaattctcacctttcaagagcttattcgagtgcacgcattgggaacgaagcattagttgatgtaattgcagcttgtcagggtcctgaaggcaagggctggtttcaggtaaaaaacaagtgacaaaaaacaggcagttttgagcctttgcttcctgcatcttttccaaatatttataaatgcttgctcaaacattcccccttgagtagttgttaacatgaataagagaagcactttatatccatttctatacattcaatgcattgtacagataaaggaatgcatagagttgaacatagtaaactaagaatttgaaagattttataggttcttctatccatcggaataatcagaccgtaataatgatttatgttttcttgtattctcagggaactgctgatgcgataagaagatgtttgtgggtactagaagagtatccaattgttgattttctggtacttcctggttaccatctttacaaaatggatttccaggaactcttaaaggtccactgcaacaatagagcagatataactgttgcagtgttaagcacaaggagggataaagatattgaactttctactttccaagtaaattctaataatcaagtcatttcattcaaagacaatccagagatgagagtatgtcgctttttctttctcctcatacttccaattgcttcatttgttcctcattatggactagcatagaagttcttttccttaaaacaatatcaaaacattgtttcaggtagcaaatttagacgtttctcaagataagtttatcggcatggatatctatgttgttaacaaagatgccatgataaagcttctgacagagtatcaccccatggccaatgacttcagaagagaagttgtaccaggtgcaatttccttaggaatgaatgtaagagtcgtgtattttcatatctgagcaacttgttgctccaagttgccattctttaagatttttatttttttttccaaaaatttgaacataaaatatctccatcctgctgcgcaaagatattcaacattttgtcttcactctaattgaactgttcttttggaacaaacaggtatacgcacacaaatttagcggctactgggaggacatgagaagcattgaagcttactaccaggcaaatatggaaagcataaagaataccaataaggcatataggtcagtatcttcagaacacaagaagtctctagagtaaatgcactggatcccttaaatttcacctaacgaaattttcattttcaatagcagcttgtatgacaaagactcccccctatacactttgcctcggcacctgcctccgactcagataactgatgctgctataacagactctgtgattggagacggatgctttctcaatgtat

SEQ ID NO: 38 - DNA, 1461 bp, Capsicum annuum
  source 1..1461: mol_type = genomic DNA
atgtctcttcctacttggttgaatctcaagattaagtcccctcagtatggctataagcctacatcaaggatcattccagcttctgttccatcaaagttgaatctatcatatacacaacactctgaatgtccaaacaatctatgtcctccagcagatcagaatgttgcagcatttgtgtttggagatggatcaaattcaagactttatccattgacaaagagaagatcagaatctgcacttcctatagcaggaaactacaggctcattgatgcagttgtcagcaactgcatcaatagtaatatcaagaagatttatgcacttacacaatttaactctgcctctctgaattctcacctttcaagagcttattcgagtgcacgcattgggaacgaagcattagttgatgtaattgcagcttgtcagggtcctgaaggcaagggctggtttcagggaactgctgatgcgataagaagatgtttgtgggtactagaagagtatccaattgttgattttctggtacttcctggttaccatctttacaaaatggatttccaggaactcttaaaggtccactgcaacaatagagcagatataactgttgcagtgttaagcacaaggagggataaagatattgaactttctactttccaagtaaattctaataatcaagtcatttcattcaaagacaatccagagatgagagtatgtcgctttttctttctcctcatacttccaattgcttcatttgtagcaaatttagacgtttctcaagataagtttatcggcatggatatctatgttgttaacaaagatgccatgataaagcttctgacagagtatcaccccatggccaatgacttcagaagagaagttgtaccaggtgcaatttccttaggaatgaatgtatacgcacacaaatttagcggctactgggaggacatgagaagcattgaagcttactaccaggcaaatatggaaagcataaagaataccaataaggcatatagcttgtatgacaaagactcccccctatacactttgcctcggcacctgcctccgactcagataactgatgctgctataacagactctgtgattggagacggatgctttctcaatagatgcaagatcagaggtacagtggttggcatgagaacaagagtaggagatggagcgataattgaggactcagtaataatgggatctgatacttaccaagtaagacacatgaattcatttcatttggcattcgaggatatgcacattcctattggaattggagaaggctcacagataaggaaaacaataattgataagaacgtaagaatcggcaaaaatgttaagattttgaataaggaaaatgtccaagaatgcaataatgaagcaaatggttacattatcaccggaggcatagttgtaattctgaagggtgctattattccagatgggagcattctgtga

SEQ ID NO: 39 - protein, 486 aa, Capsicum annuum
MSLPTWLNLKIKSPQYGYKPTSRIIPASVPSKLNLSYTQHSECPNNLCPPADQNVAAFVFGDGSNSRLYPLTKRRSESALPIAGNYRLIDAVVSNCINSNIKKIYALTQFNSASLNSHLSRAYSSARIGNEALVDVIAACQGPEGKGWFQGTADAIRRCLWVLEEYPIVDFLVLPGYHLYKMDFQELLKVHCNNRADITVAVLSTRRDKDIELSTFQVNSNNQVISFKDNPEMRVCRFFFLLILPIASFVANLDVSQDKFIGMDIYVVNKDAMIKLLTEYHPMANDFRREVVPGAISLGMNVYAHKFSGYWEDMRSIEAYYQANMESIKNTNKAYSLYDKDSPLYTLPRHLPPTQITDAAITDSVIGDGCFLNRCKIRGTVVGMRTRVGDGAIIEDSVIMGSDTYQVRHMNSFHLAFEDMHIPIGIGEGSQIRKTIIDKNVRIGKNVKILNKENVQECNNEANGYIITGGIVVILKGAIIPDGSIL

SEQ ID NO: 40 - DNA, 8733 bp, Capsicum annuum
  source 1..8733: mol_type = genomic DNA
agatgaataattgtaaaaaaaaataaaaaaattcaaggtaacatgttcacttcaggaaactagcacctttaaacttacggctatttggccaaaaaattggaggaaatccaaggttcaccacgttagataatggtaccactgggaattagtgcatcctttataacagtaacaagcccgctatgaatgaagtatccgtccgactccctggctgcttcttgaacattgtcactattgatgatctgatggtacaaaactttcgtgttaggaactcaagctcacacatgtatattggttcaaaaattagaaggaaaaagcatgtgatgatgagagaagtcagcaggacacttacgactatatgcagcctagaatactcaatacatgcattgagtgaatgtgattttgaaacataacaaaagttgatacgggcacaactataaagatcgatgagtgtacaagtgtgacaagaggccatacaaggaagtgcaatgctaaagtgtgaggaggaccaaaatgcacactaaagccgccccactctacactagagaggcgccccttagtcattaagggtcttttgaacaattcactttgttatgtaatagactatatatagtttagtattagggttttcattccttagttgattattgttgtaagtcttgaactttgaaacaccaagtcctctcttcaattgaggcaaaatcgaaattaggtcacaagtgtggatatcacctgtgttgcaattgttggcttgaaacgttagtgctagaggaggttgtaaggatcattaggtttgacatatctgagggttcttatagtctatgcactcctttgtcaatctttctatccaccttatctgttgaaatcgtctcttgttgtgtttagcgttctcttgccgtttttagtgtcttgttcttgtggactgttttgtgcgagctgtggactgttttgaggcttcttaggaccgtttggaatcatttggtatcaaagctaaggctgattttgttcctacaacaccaatcttgagcttgattagttgaaaaataaaaaaaaaagttgtctaagtgttggtgttcttggacgaagtgttgttgttgttgttgttgtgtttagccgagatttgtgtgtagatctaggtgtgttttgcttgtcttagttgtttctagtgttggttttctttctctctaacacttattgagtctaaagccatgttcttgagctaagttttaagttgttgttgaaggttgttagacggcttcttgttgttgttcttcttgtccttcatgttgtgttgaagaagtatctttaaaaggagttgtttgatcttgaaaaaagaaaagaaaagtgagactttgtgtttgtcttggaaacattttcaagacatcatcaaagtagtaaaagaacaaaagacattcaaaaagtttagttactaaaaatgagtaagacactttttcaagattgtccaaaaggagaaaggggacaagacatccaaaaaggttgtcttgccaaaagagtgtaagtaggtgaaaagcttttgactcttgaaaagagggtctaaaaccttatatacctacacacatagccgaaacttcttcatcaaaagtgtgaaattgaacaacttaattcttggaacaaatttcttcaacttcaccaacatttccaatgaccaatagttgttggccacgtcatcaacaaggctcaaaatttgggaattcttagtttctcttctttgtttccattggtctcaactttgttgactcttatactaattgacaactagtaaatatctactaagttgtaaattagtttttgagtacgttctttgtgttagcattatttttgtgcttttctcagttttgagtcgtagtatttcttggttcaaggtcggacattactttcttgagtcttcaaaacaaagaattcattccaatcaagaagtagacttactcctcaactcaccacgagctacaagtcaacttgcaacacttgtgacatcaagtgtgagacgacaaaagtgcgagagggtgtagtgaggttgttttgaactaacgcatgttttgttttgtaggttttgtgcggtattgttgttggctggttcaagaacttgaactttgttggtgttgttgtgtgtgaagttatggactgttttggtgtttggttggaccatgacttaaaggtttgccttaagtataatcgtaaatagaacccgaggcttcaacctctcaatccacgatgaacaataatgccattaatgctatattgacccgtcttgaagccatgtcccgtgacattgctagagtgaatgcgagtattgaaaatttaaactcggacatggcatcgatgaagggtgagatgtcgactatgggtggaaggatagaacgagtggaaagccaaagaagccgactttctacccctcaccaactaatactcccgaggccttgcaccaaatgttatatccaccaattgccacaaatcaaaccaatccttattcccaaagccgggatcatgataggccaagtcactatccacttcaccaagtccaacaagaaggacttggaacctaaataccaccccaaaacccgagtcctccattccacaccccactttaccaaagacctcactatccaacaaatccaatcccaccaccacaagctccacttaacccaaatagacccgtccatgtagaagggtatggtagaggagatcaacatgaaggatattattcgtatgatgatatttagatgagaagagaggagatggggagtaggcaaggtgatccgagagaagtttatggtcaagagggaggagttggttattggaaccaaaaccgtgatgtaggcttaaataccatcaagatgggactttcaatctttaagggtgagagtgatcccgaagagtttcttgcatggaaatccgcatgagagagcatcttccaagttaatgatcttaccgaggagaaaaagagttgctatgtgatcactcattttgaaggttatgctactacgtggtgggagtatgtaaaacggtttgataatgttttgatagaggggcatccccatccttggtttcagttaaggtacctcatgagacaaaagtaattacccgaaagttaccaacaagagttcctcgccaagttgtataacttgaggcaaggaaacaagagcgtcgtggcttattacgatgagttccaacaacttattttgaaacttgatcaccgaggaagcaagtaatccatgacattgttcgattcaaggttggattgaatagggatatctcctcacgattgacacttcacaaattcgacaccattgatggcatcttccaagccgatttggatgttgaaagggagttaaaaaagagatcggggtacaagtacaaaggaaaatctcctttgggttggcacaaggacaaggagcactcattaaatgccgaacaacatgatgccaaggctatccaagccgagaacaagacctcccaaaggtaccctttaagaaaagaaggtaaaccacacttctcgtctcattctaagggctttcaatgttttaaatatcaaggttggggacataaggatagtgagtgtccaaataggagaaatgtgatcctaagagagggtaaattatactttcttgtagaggaggtgggacttgaactggatgagattgataaacaatctcaagatggagagaagggacaagttgaggggtcaaaagaaaaaggatgtttggccgtataatggtgagctcaaagttccatatttggtggtgagaagagccatgattagtaaggctttggttgatcctattcaaagggagaacctttttaacaccaaatgccttattaaggaagttgtgtgctctatgatcattgatggaggtagttgtgccaatattgctagtagtactttggttgacatcttgaagctgcccacaatccctcataatagtccatacaagcttcaatggttgaatgcgtgtagggagttaacgataacccgacaagtaatgatacggtttaaggtagacaactaccttgacgaggtgttttgtgatgttattccaatacaagcgtaaaatttattgttggggataccatggaaatatgatagagctagcatgcatgatggaagaaccaataggtacacttttgagctccatggccgaagatatacactccaccctctcttactgttacaagtgactaaagttcatcaacaagtgaaagagttggaggagaagagaaagaagggaaggagagggaaaaccgagagtgtaaatagtgaagaaggggtggcagcaaaggttcaaggaggaaagaagtccctacttgtgggacttacccgcaaggctccctaatttctcactaacaaaggtactagcattttgcctagtaccgtcttttcttgtgtataggttcatgatcatgtgcttcctaaggaggataccaagaggattacttaccaacacatgcttgatgatgtcaaggagcccttggataagcatccaagcttcaggatagaaaataaagtcttgattcaaagaagaagccttaaaggtgatgaagccgtgaatttgaggccaaattcctttcaagatggagagcatgatacgggcatgactatgaagatcgatgagtatgaaagtatgacaagaggccatccaaagaagtgcaatgctaaagtgtgaggaggaccaaaatgcacactaaagccgccccacgctacactagagaggtgccccttagtcattaagggtcttttggacaattcactttgttatgtaatagactatatgtagtttagtattagggttttcattccttagttgattaatgttgtaagtcttgaactttgaaacaccaagtcctctcttcaattgaggcaaaaccaaaattaggtcacagtgtggatatcacttgtattgtaattgttggcttgaaacgttggtgctagaggtaacgttcctctagtgtcttcgtaggatattggtgatagtttgtgtaggttgtaaggtccttaggtttgacatatatgagggttcttgtagtctatacactcctttgtaaatctatctatccactttatctattgaaatcgtctcttgttgtgttttgtgttctcaagccgtttttagtgtcttgttcttgtggactattttgtgcgagttgtggattgttttgaggcttcttaggaccatttggtatgaaaagttgagcggtaattagaaataaattaggtagccaaccttcacattgtcccctatacgagcattcttgtcgataatagctctctttatgtgagagttcttgccgatgccaattggaagactaccctttgcatacagaagtatcctgtcagcattcgtctggaccaagaaaggaagcaaagtgagtttaacaacaaatcgaaggaggcacaaagaaaattacagaggaaattactgtcgtttttcaatttgctacatgctatctcacgtacaaattaagtctagtcaggtgtgctgagaactattagtccttcgtgaattttaactattacctcataataatctgctcccatcaaaagtgtgtcttcgataattgcttcctctgaaatgcaagatctgagtccaatcacggaatggtgaattttacagctctgcactcaacaaattacctccgtgagaaccgtgatctgagaagtatgtactggtgttcatgtactgtttcacatagaacaaaccaatactccctcttccaaattgttttttgctcgttcctttttttcagttgtctcgaaattgtgtccattttcctaaatcatacatttgttttccgctatttgttcttttattttggaatctatcattcttataaaatctcaaactcatcaaagagtccacttcacacttacggtccatttcgctctatgctatgttataaattaacttcactaataactcaaactaagagaaacaaattggaaaatagggagtaacggtccggtaattaacatcacaaattttgacacaacataatttatgatgtaaagaatatatcttccctagcttgatgggcaaattaaactgaaactgtgaaacatgacatgaaactggaaaaaggatgttcaacattcctcagaaaatgcaatgcaagcaggaacctttttttgtgacttgtaagtataagagatgtcccaagattatagggcacctctcctggacagccagctcagcgggtaattgaggcgaagtcctacatgtttgctgttgggcacgccatgtataactaaaggcataattcataaatatttggcctctgctggcacgtaaattttgagtatacaaatctagacacctcaactcatatccactatgtcagttgagcactccaactaataaaatgatcatctaaacacctcaactccgatccactatgtcagttgcacactccaacttataaagtgatcatttagacacctcaaaaatttgtgcatcacattggcatcaggtgaccacaagacacagtggggacgagttggagtttttagttgatagttgagaccaagttgaggtgtctagatgtgcgttctcaaaattggagtgcttacttgccaattgatgccaagtttgagtgtttgtttatgtattatggtataactaaaaatgaaacgaatatgaaaattagaagaccctgttggtgagaaataccttgataacacaaccttcaccaatgacactatcagtgatatcggcatcaagcatctttgaaggaggcaaatatcgaggttgggtatagattggagctgaacgatcatagaagcttcagggaatattgaagataatgatattagtgcttacaatcagtgcagaacaatcgacaatagtaaccaagtgcagagaaatatcagtgaagttacctaaaatctggcactggcttttttgtaatgcccaaattggcattgtagaaagcttcaatggtaccaatatcctcccagtagtcatcaaacaaataagctttcacctgaacttagactagtaagatctctacaagtaatactaaggaaattttcggctgataatttgaacgtgtgagtacaaattttggttcgtcattatttcaagcagttccatgatgaccagaaagagagaaaaatatacgatgcagagatgcggtaaaaagatagaggaaaatctgatagcggttcacaagactatagaaagatggctaatttatacaaaatcatactcggcagaaaaaaactatgttgctcgaactaataaaaaatgttgacagctaggagcctgatcctccataagtggtgcatttttgaaggattcgacacaggtgcagcatcttttttggagagtccaagcaatataagaaaaaactaacagctagccataataaggcctaccaagggcggacccaccttatagcaaggtgtgtcgtccgatgcttcttcatcgaaaatattaatcaatagatacatcaaatctggggaaaaaaaacagtttgtatataaatatgcaatgacacagctttagcaaagagcactatggtctgttcgttaggcgcctatctaataaggttctctcttggacagaggtggatccagaattttaaacttgtgggtttccaagggctcctttcctgccactaagctatcccttggtttagttatgggttcccacctattaatatttataacttttggcgatttttctatgtaattttaagccttacggtagcgagtgtgggttccnnnnnnnnnnnnnnnnnnnnnnnnnncatccgccactgctcttggagatcgaaccccattggcagcacaattttgagttttttgtttctgtaggtagtaattgctaaaacatagcaagctattattagttaataggatttgaatcccctaaagctcactcctcaagctaaacctaaaatctaacgtcaaaaaaaattaaccattctttgccctattttgttcatctattttctatgttaaatgtcgacactgcttataaaaaattctagtatgccactgaggcgtacaactacttgagtaatctggaagtgaagtttcatatttcagaatttaaattctattcatgagaaaaagtaaatttcttccagtgtttgtaattagaaccgtaaatggtaatcacttaagaatctacatactcgcaatccgatggaagttgcaccaggtataacttcactaccaaagtcatttgctgcaggaaacctttcccgaagtaggtttaacatcacatctttgcttacgacataaatacccatacttgcaatatatggcttctctctagctttttcgtcatcaaggcctaaaatagtagtatccacctgatatgcaccaaacacaaattaagttcttccataaagctagatatttgtaccatctagtatacagagataaatgtatccgcatgcgtatttattgagaaatatgcagaaggacttacctgcattgctttcaactgctctccttttggtttctctgcaaattcaatgatgcgtccatcttcatcaatcttcattagaccaaatgcagaggcacgcttttcatccatcggaagtgcagcaacggttatatcagcatccgattctctatgggcttgaatgaacttttcataatccattctgtacagatgatctccagcaagtattaggtattccagaacattatgctcctcaaacaaccacaaatactgtctgacagcatcagcagtgccctgaaagacaatt

SEQ ID NO: 41 - DNA, 1026 bp, Capsicum annuum
  source 1..1026: mol_type = genomic DNA
atgctttttctaaagccatttaggggcactgctgatgctgtcagacagtatttgtggttgtttgaggagcataatgttctggaatacctaatacttgctggagatcatctgtacagaatggattatgaaaagttcattcaagcccatagagaatcggatgctgatataaccgttgctgcacttccgatggatgaaaagcgtgcctctgcatttggtctaatgaagattgatgaagatggacgcatcattgaatttgcagagaaaccaaaaggagagcagttgaaagcaatgcaggtggatactactattttaggccttgatgacgaaaaagctagagagaagccatatattgcaagtatgggtatttatgtcgtaagcaaagatgtgatgttaaacctacttcgggaaaggtttcctgcagcaaatgactttggtagtgaagttatacctggtgcaacttccatcggattgcgagtgaaagcttatttgtttgatgactactgggaggatattggtaccattgaagctttctacaatgccaatttgggcattacaaaaaagccagtgccagattttagcttctatgatcgttcagctccaatctatacccaacctcgatatttgcctccttcaaagatgcttgatgccgatatcactgatagtgtcattggtgaaggttgtgttatcaagagctgtaaaattcaccattccgtgattggactcagatcttgcatttcagaggaagcaattatcgaagacacacttttgatgggagcagattattatgagacgaatgctgacaggatacttctgtatgcaaagggtagtcttccaattggcatcggcaagaactctcacataaagagagctattatcgacaagaatgctcgtataggggacaatgtgaagatcatcaatagtgacaatgttcaagaagcagccagggagtcggacggatacttcattcatagcgggcttgttactgttataaaggatgcactaattcccagtggtaccattatctaa

SEQ ID NO: 42 - protein, 341 aa, Capsicum annuum
MLFLKPFRGTADAVRQYLWLFEEHNVLEYLILAGDHLYRMDYEKFIQAHRESDADITVAALPMDEKRASAFGLMKIDEDGRIIEFAEKPKGEQLKAMQVDTTILGLDDEKAREKPYIASMGIYVVSKDVMLNLLRERFPAANDFGSEVIPGATSIGLRVKAYLFDDYWEDIGTIEAFYNANLGITKKPVPDFSFYDRSAPIYTQPRYLPPSKMLDADITDSVIGEGCVIKSCKIHHSVIGLRSCISEEAIIEDTLLMGADYYETNADRILLYAKGSLPIGIGKNSHIKRAIIDKNARIGDNVKIINSDNVQEAARESDGYFIHSGLVTVIKDALIPSGTII

SEQ ID NO: 43 - DNA, 4395 bp, Capsicum annuum
  source 1..4395: mol_type = genomic DNA
ctccttaattctcagaggcaaaaatggctgttgctgtcgacgggcgattcgcgttagttcgtagtaaccctgcaacattgacgacagggacaaacgtgaggttagcgaaggcctgcaatggagagttgatggggaagaaactcaagtttacaaaatttcagttaaggagcaatgcagtgaaacctaatatctgcatgtcacttactgttgatattgcaggtgaggctaaggtgagttttctacgtttacattttgacccccacctgccagtgccatatatttcttggcttcctttataattgaatgagcattggaactactttaataattaataaagagatgggccatctttcattataaaatgtgacttgatacacaagtaagatcaaatgatgcgtcataaaacaacttaactgatcaaccaacatgtgaaagggcagtccaatgtattaagatctcgctacgtaggaatccaggaaaggactaaactacaagggtttattgtacgcagccttacctcatatttctgcaagaggctatttccacggctctttgtagggtaaggctgcgtacaacatgtaattgaactcattaataaatgtaggaagcatccaacagatgtaatcacaccataccattatgttgtagtgactgttgtggcattggaccgtaacttaaaatcttaaattcgccccgaaactttaaagaatggtcattttgaccaagtgccgaatgctgacagctgaaggatcttgaaccaaagaaggaggatgcaaggacagtagtagcaatcattctaggaggtggagctggcactcgtcttttccccctcaccaagcgtcgtgccaagcctgctgtaagttttcttcagattatcagaaaaaatattgaaagttttcgcagagctatctgcatctcaccattgagtcttattaattcattttaggttccaatcggaggagcatacaggctaattgatgtaccaatgagcaactgtattaacagtggcatcaacaaagtatacattctcacccaattcaactcagcctcacttaacagacatatcgctcgtgcttacaactttggcagtggtgtcacattcggagacggctatgtcgaggtaaacaccaacgttcatttccagtatgcatacatttggtttcaggttttgatgatattgtagcacaggtcttagcagcaactcaaacaccaggtgaattaggtaaaagatggttccaaggtactgcagatgctgtgaggcaattccactggctttttgaggtacacacatggttacattgaattgcaatctccatgatttatcctaaatgacggtgcaagtaaaaacagattcctgaactgcaatatttctgtcttcctcataatcgtcaaattaccatgctttcctcccagttttttcttcaaaaagtaaaaattatctaagaatcaaaatgcatatattcacacatcatatcaggagattccatttataatcgacctgaatatcgttgttggtggcctagaaggaagctcaaccttagacacggtccaataatagtttacatcctcaacagattacccatgcaaaatcaagctagctaagttgtaccaatttagttccggccaggaaacttacaaggaaaatacagtacaacagcatgtaaatatcagcaatactttttctacactcgctattcccatcccaaaattatctcatcatttggctcagttacatacataacattgattcttgcaatgttcacactgattttcaacttcttaaggatgcaagaagcaaggacatagaagatgtgctcattctctccggagatcacttgtatagaatggactacctggcctttgttcaggtatagactagactaagctatacgcaccttgattgataacatatatcctaaaattacctatttctccagattgatatttccatcttcttgacagagccatcggcaaagtggtgcagacattaccatatcaagcttgccaatagatggcaggtagcattaggagggacaatctagatactagatagactattcttcccctcaacgaaatcatactttctcaaagactgctttgaaaatcactacaagaaataatgtcttctattgaatcacttctatgttttcatgatagacgcgcttcagattttggcttgatgaaaattgatgactcaggacgggtcctgtccttcagcgaaaagccaaaaggagatgatttgaaggcgatggtaattcttgaaaatttgacagtctcagagcttaatctacttttaaattataaagttgctgcaatcacttgacaacaagcacttcaggcagtagacacatctgttctgggattatccccagaagaggctaaacagaaaccttacattgcttcaatgggagtttacgtcttcaagaaggatatcctcctgaatctcttaaggtatgtaacgagtttcttctatagtaccttactttagagggcgtcattgtcctgccaacatgcaaatgaaagaaacttggagaaagaggacaaaaaagtgaagaaactatatgcatccatctaacaggggtagccacactcattttggtggttattataaagtagaaagagaccgcataaaatctatgttaaccctgagttccttcatacttttgcatgctgaagagatcaggtaaaagaagatcgtgaactgctaactctttgtctttttagatggcgttttccgacggcaaatgactttggttcagagattatccctgcctctgccaaagaattctgtatcaaggtgggtaccacttactttctggttggcatttatgcaagacctcgatctgttggctcaaaacgttcactactacacaatattgttgtttcatcatcacaaggcatctaagatattctatgcgcatttggtattatattgcagttcacgggcccacaattcctctccccctgtttatcttatttcccaccctcacccctgcatgtcttcattgtcccaaaaaattcaattcactatttctgttatggtttcctaagcaacttaactacaacctgtacagtgactaaggcgcattttgccctgattttgcaggcttacttattcaatgattactgggaagatattggtacaatcagatccttttttgaatcaaaccttgcactcactgaacatgtaagaatcagacagctatattatatgttttttcagtcttttaaggggttttgtttcttcaaacaacccaagtaagatggaatctggctattgaacacctgtcaatcttacttgacaacattaaaaagattgttcgtactaataatatggccaactaatgttgcttttttcatttcagccaccaagatttagtttctatgatgcaacaaagccaatatacacatcaaggagaaacttacctccatcagcgattgataatagcaaggtttaaagcacaaacccacttcttcaatcttaaacacatacatctctaaattttcttctgctaaccaaatctagttgttgccagattgttgattcaattgtatcacatggtagtttcttgaccaattgtttcgtggagcacagtgttgtcggcatccgatcccgtataggcaccaatgttcacttgaaggttagtctaccaaaactgcatatgatgctattgaaaattcagtattattcttttgaacaaccaaaccaaaattgatgctccaactggaactcacgtacatccatctaacacacatacatttatgtttcatatttaaacaattaaacacatttaagtacttctgcatttacttgatttttataattggtgctaattcctcataacttcgttaaaatccatacaggacacagtgatgcttggtgcggactactatgaaactgatgctgagattgcctcactgctagctgaaggaaaagtgcctttaggaataggagagaacaccagaataaagtaattcactaagatgtttctccttttgtgtctatcaaacaagaaaatgaatgtgacctttatttcatttctcacgttatttcgactaatatgcagagagtgcatcattgacaagaatgcaaaaattggaaagaatgtagttattgccaactcagaggtatgatcctcctatactcccaacaacagcagagaacaaagtttatcttgttattattgggactcacttattttccgctgatacatcaatttgcaggctgtacaagaagcagacagatcgtcagaaggattttacatccgatcaggcatcacagtcatattaaagaactcaacaatcccagatggggctgtgatatgacaatgtctttttgtcttttctttcaaatagcattggcaccaagaaaattgcgaaatatgagaagtttca

SEQ ID NO: 44 - DNA, 1551 bp, Capsicum annuum
  source 1..1551: mol_type = genomic DNA
atggctgttgctgtcgacgggcgattcgcgttagttcgtagtaaccctgcaacattgacgacagggacaaacgtgaggttagcgaaggcctgcaatggagagttgatggggaagaaactcaagtttacaaaatttcagttaaggagcaatgcagtgaaacctaatatctgcatgtcacttactgttgatattgcaggtgaggctaagctgaaggatcttgaaccaaagaaggaggatgcaaggacagtagtagcaatcattctaggaggtggagctggcactcgtcttttccccctcaccaagcgtcgtgccaagcctgctgttccaatcggaggagcatacaggctaattgatgtaccaatgagcaactgtattaacagtggcatcaacaaagtatacattctcacccaattcaactcagcctcacttaacagacatatcgctcgtgcttacaactttggcagtggtgtcacattcggagacggctatgtcgaggtcttagcagcaactcaaacaccaggtgaattaggtaaaagatggttccaaggtactgcagatgctgtgaggcaattccactggctttttgaggatgcaagaagcaaggacatagaagatgtgctcattctctccggagatcacttgtatagaatggactacctggcctttgttcagagccatcggcaaagtggtgcagacattaccatatcaagcttgccaatagatggcagacgcgcttcagattttggcttgatgaaaattgatgactcaggacgggtcctgtccttcagcgaaaagccaaaaggagatgatttgaaggcgatggcagtagacacatctgttctgggattatccccagaagaggctaaacagaaaccttacattgcttcaatgggagtttacgtcttcaagaaggatatcctcctgaatctcttaagatggcgttttccgacggcaaatgactttggttcagagattatccctgcctctgccaaagaattctgtatcaaggcttacttattcaatgattactgggaagatattggtacaatcagatccttttttgaatcaaaccttgcactcactgaacatccaccaagatttagtttctatgatgcaacaaagccaatatacacatcaaggagaaacttacctccatcagcgattgataatagcaagattgttgattcaattgtatcacatggtagtttcttgaccaattgtttcgtggagcacagtgttgtcggcatccgatcccgtataggcaccaatgttcacttgaaggacacagtgatgcttggtgcggactactatgaaactgatgctgagattgcctcactgctagctgaaggaaaagtgcctttaggaataggagagaacaccagaataaaagagtgcatcattgacaagaatgcaaaaattggaaagaatgtagttattgccaactcagaggctgtacaagaagcagacagatcgtcagaaggattttacatccgatcaggcatcacagtcatattaaagaactcaacaatcccagatggggctgtgatatga

SEQ ID NO: 45 - protein, 516 aa, Capsicum annuum
MAVAVDGRFALVRSNPATLTTGTNVRLAKACNGELMGKKLKFTKFQLRSNAVKPNICMSLTVDIAGEAKLKDLEPKKEDARTVVAIILGGGAGTRLFPLTKRRAKPAVPIGGAYRLIDVPMSNCINSGINKVYILTQFNSASLNRHIARAYNFGSGVTFGDGYVEVLAATQTPGELGKRWFQGTADAVRQFHWLFEDARSKDIEDVLILSGDHLYRMDYLAFVQSHRQSGADITISSLPIDGRRASDFGLMKIDDSGRVLSFSEKPKGDDLKAMAVDTSVLGLSPEEAKQKPYIASMGVYVFKKDILLNLLRWRFPTANDFGSEIIPASAKEFCIKAYLFNDYWEDIGTIRSFFESNLALTEHPPRFSFYDATKPIYTSRRNLPPSAIDNSKIVDSIVSHGSFLTNCFVEHSVVGIRSRIGTNVHLKDTVMLGADYYETDAEIASLLAEGKVPLGIGENTRIKECIIDKNAKIGKNVVIANSEAVQEADRSSEGFYIRSGITVILKNSTIPDGAVI

SEQ ID NO: 46 - DNA, 4539 bp, Capsicum annuum
  source 1..4539: mol_type = genomic DNA
acttaattgatagtgaaagtgagggagtatggaatcgttgtttgctaatatgaagggatcattgacaacaatcataaacaatcaagaggatgcattttggggagagaaaataagtggaagactgataaacaagggtttccgatggtgcaagagctttagaagcatggaaaggggtagaaaatttaaggctggattcgccttctctgttctcactcgtgatataaacaaaaacctactggttagtagaggaattaagtgatgcttctttcaagtaaagttaatgatccatctaatttttgttggtttcagtcatttgaagattcaatctttgaggaggcagacccaaaagctgttgtgtctgtcattctaggtggcggtgctggaactcgtctttttcctcttactagcagaagagctaaaccagctgtaagaattacaacacatatctttgcttaaatcttggttggtaccccattctgtgtcttttgaggttgaaaatatgtccaattcaaaacaacttcagcctgaaattactagtggttcttaactccaattcagctggatttttgaaaactaaagcagctcagcgtggattttccttatctgttctcactgaaattaagttttaagaatgtacttacatgttcatctctattggttttcattcatattgtgtcactactgtatttgttcttcaggttccaattggtggttgttatcggctaattgatgtaccaatgagtaactgcattaacagcggcatacggaaaattttcatcttaacacagttcaattctttttccctcaatcgtcaccttgcccgtacatacaattttgggaatggagtgggttttggagatggatttgtggaggtaagaccattctggttacttgatgttcttagtctaacctattttaagctacctatattttggcagtgaaatttaatcatatctttttaagaacacacttttatgagatgcttgctgagtgtctatttgtaaaagctttgttaatttccattcctctggctccttggatccacggggaggtaggttgaacaaccaaaaaaggggaagataaaagaaatgtgtcctggacagataaaacaaggttttctctaatgcaggttttagctgcaacccagactccaggggatgaagggaaaatgtggttccaaggcactgctgatgctgtgcggcaatttatatgggtatttgaggtaggcaaataagatcttccattgataaagctcctaggaaaaacactgaccttttatttcaaatggtgacatagaatcagaagaacaagaatgtcgagcacatattgattttgtctggtgaccatctttatcgaatggactacatggactttgttcaggtatatgagcaacttgtgacaacaattgcttcctacgctaaccctatgttcatatattttgctgaatgaagaatatttgtgcttgcagaagcatattgacacgaatgctgatattacagtttcatgtgttccaatggatgatgggtatgagattgatctttcttctaacttgatagcttatttcatgtgaagccaaatcctatttgcgttgctttctgtcttgctgaattccttgaaaggttctttctaatgctgttcagaatggcaaatggcagccacagttgttcagaatatgttgaaggcatattgtcacttgaagtggcaatttatattagacatttcaatctacagataaaattggattaatcttcaatacgattttggcaacgacaaggaaagagaattagcaaaagaaaattagaattgtagaaagtttgaaatcctttttagtttaactatttaagtggacctagtatcttttttatcatcaccatgacagatagataataataatactgatgcattttcccatccgacatccttattaactttagagcaccagcactgagtaatatccactgaaagatctggaagcatacagaattacactttgcagtttccgttaagtctatttctgttttgatttctcaaaaaaagaagcctatttctgttttgagctcccaaacttttatatcttccagatttcagtaatgttgtctatctagaatgcactataaagactaacaaacatttcaaactatatcaaggagttaatcaagacatttctctctctcttacatccctccatcctcccttttggcagtcgagcttcagattttggattgatgaaaatagatgaaacaggacgtatcatccaatttgctgagaaaccaaaaggtcctgctttgaaggcaatggtacatatatttggacttccacaccaggaacttctattaattaaatattcccttacagccgctcctgctttcacattgtctcttctcctatgtaacttacgttgtacgtctaatattctattgcagcaagttgacacatctatcctcggactatctgagctagaggcagccaattttcattacatggcatccatgggtgtttatgtattcaaaagtgatgttctactaaaacttctcaagtcagcatacccttcatgcaatgactttggctctgaaattattccttctgctgtgagggatcacaatgtccaggtaagagctggtcgttacttcctcaattgcaattttagtatttcgaaagttcataaattttgctgttcattgtgtacttagactttgactgatcgattttagtaggcatatttgtttagtgattactgggaggatattggaacagtgaagtctttctttgatgccaacttagccctcacagagcaggtgagcaacttaatgtgtactaccttctgactgactacttctaagagtacacaatgtaagagctagacagttgatgcaaggactctgaaatatggagcagtagtttatgtaactgtggaatgctacatctcagtgaattttgtagttatgtttcgttttatataccataatctctaccttcagttattgattaataacttgttgctgaattgcaatttcctcttttctccctgttttatgtagcctccaaagtttgacttcaatgacccgaagacaccattctacacttctgctaggttcttacctcctacaaaagttgacaaatgcagggtatggaagttataacataaatactttgtaaaatttttccaggatttcaggttactatgaacacgtcatgttaatactgttcattgtcagatagtggatgcaataatttcacatggttgttttctgagggaatgcaccatccagcactctatagttggtgtgcgatcacgcttagactatggtgttaagtttaaggtatgttttcatattctgattgaaagcttatcttacctgtttttagctggaaagttctatcagtaagatggcagcccgctgcactaaatctcccactatgcacggtgtccgaggaagggacgaaccactagggtctattgtaagcagccttaccttgcatttctgcaagaggctgtttgcacagcttaacccatgacctcctaggcacatgacgacaactttaccatttactccaaggctctcctgttagagcctggactaaattaaaaacaacaccctgtattcatctgatggaattctgacaacttagaaaccaagatgacatgcaaaggcacataactatcctgtggatgtgccatcaaactagttgtgggaaggatgatttatgacagtcatggatattttttgtagatatgctttaaagattggattcctcctgtattcttgctttctgcaaataagctggtaaaatgtactctaggaatggactttagatagcggagcaatgaacgagaagcatgatcttagcactcagatgctttcttcaaaaatagctatgtagtgcttacttgtttgtaggctgattatgtcatacacttctcaggatacaatgatgatgggtgcagattattatcaaactgaatctgagattacttcactactagcagaaggaaaggttccaataggggttggaccaaacaccaatatacagtgagtttttttactgaactcatcctctctcctcttgttgattgaaaattgaccactcagacttccattttgactgaccacttgaaactttgagctctggtgctactgattcgtccatcactattcattcctatctaatttacagttgtttgtctaaacttcaacaagtttaccttagaggactttgacatacttttctttctattttcttgaagaaattgcataattgacaagaatgcaaagataggaaaagatgtggttatcctgaacaaggaagtaagtatctttcttctgaagtttaaaataatcataaaagtgaatacaagtttacctgatgctttgatgttgcagggtgttgaagaagctgatcggtcggcagaaggattttacattaggtccgggattactgtcataatgaagaatgcaaccattaaagatgggacagtcatatgaaaacttgtaaagtgttcttgctagtggatcaagttgatcctcggaggaaataaaa

SEQ ID NO: 47 - DNA, 1557 bp, Capsicum annuum
  source 1..1557: mol_type = genomic DNA
atggaatcgttgtttgctaatatgaagggatcattgacaacaatcataaacaatcaagaggatgcattttggggagagaaaataagtggaagactgataaacaagggtttccgatggtgcaagagctttagaagcatggaaaggggtagaaaatttaaggctggattcgccttctctgttctcactcgtgatataaacaaaaacctactgtcatttgaagattcaatctttgaggaggcagacccaaaagctgttgtgtctgtcattctaggtggcggtgctggaactcgtctttttcctcttactagcagaagagctaaaccagctgttccaattggtggttgttatcggctaattgatgtaccaatgagtaactgcattaacagcggcatacggaaaattttcatcttaacacagttcaattctttttccctcaatcgtcaccttgcccgtacatacaattttgggaatggagtgggttttggagatggatttgtggaggttttagctgcaacccagactccaggggatgaagggaaaatgtggttccaaggcactgctgatgctgtgcggcaatttatatgggtatttgagaatcagaagaacaagaatgtcgagcacatattgattttgtctggtgaccatctttatcgaatggactacatggactttgttcagaagcatattgacacgaatgctgatattacagtttcatgtgttccaatggatgatggtcgagcttcagattttggattgatgaaaatagatgaaacaggacgtatcatccaatttgctgagaaaccaaaaggtcctgctttgaaggcaatgcaagttgacacatctatcctcggactatctgagctagaggcagccaattttcattacatggcatccatgggtgtttatgtattcaaaagtgatgttctactaaaacttctcaagtcagcatacccttcatgcaatgactttggctctgaaattattccttctgctgtgagggatcacaatgtccaggcatatttgtttagtgattactgggaggatattggaacagtgaagtctttctttgatgccaacttagccctcacagagcagcctccaaagtttgacttcaatgacccgaagacaccattctacacttctgctaggttcttacctcctacaaaagttgacaaatgcaggatagtggatgcaataatttcacatggttgttttctgagggaatgcaccatccagcactctatagttggtgtgcgatcacgcttagactatggtgttaagtttaaggatacaatgatgatgggtgcagattattatcaaactgaatctgagattacttcactactagcagaaggaaaggttccaataggggttggaccaaacaccaatatacaaaattgcataattgacaagaatgcaaagataggaaaagatgtggttatcctgaacaaggaaggtgttgaagaagctgatcggtcggcagaaggattttacattaggtccgggattactgtcataatgaagaatgcaaccattaaagatgggacagtcatatga

SEQ ID NO: 48 - protein, 518 aa, Capsicum annuum
MESLFANMKGSLTTIINNQEDAFWGEKISGRLINKGFRWCKSFRSMERGRKFKAGFAFSVLTRDINKNLLSFEDSIFEEADPKAVVSVILGGGAGTRLFPLTSRRAKPAVPIGGCYRLIDVPMSNCINSGIRKIFILTQFNSFSLNRHLARTYNFGNGVGFGDGFVEVLAATQTPGDEGKMWFQGTADAVRQFIWVFENQKNKNVEHILILSGDHLYRMDYMDFVQKHIDTNADITVSCVPMDDGRASDFGLMKIDETGRIIQFAEKPKGPALKAMQVDTSILGLSELEAANFHYMASMGVYVFKSDVLLKLLKSAYPSCNDFGSEIIPSAVRDHNVQAYLFSDYWEDIGTVKSFFDANLALTEQPPKFDFNDPKTPFYTSARFLPPTKVDKCRIVDAIISHGCFLRECTIQHSIVGVRSRLDYGVKFKDTMMMGADYYQTESEITSLLAEGKVPIGVGPNTNIQNCIIDKNAKIGKDVVILNKEGVEEADRSAEGFYIRSGITVIMKNATIKDGTVI

SEQ ID NO: 49 - DNA, 3432 bp, Capsicum annuum
  source 1..3432: mol_type = genomic DNA
aacctattagaatccacaaaggaatagttaatgcaaaacattggcacaaatttatccacttgccttgcaattgtctatcttggttggtggaaggaacctcggagatgtgtaaaaaggtgtctttggatcgtaaaattgaaactttgggaactgcaatggaaaatcaatcagttggatgaattctataagatgaaaagaaattcatcacatatgatgagaagcatagaggggggaatttttaacatgcctctgaggtgagctccaagttagcctcataaaaagatttaattgtaccaatgtcctcccagtagtctctgaatatgtatgcctggaaaacatcataaattagttaaaaataaagtcgagctttcagtttgggtatctaatgtactcaaattcacaaaaataatttcaacagaaagatccaagatcctttacttggacattctgctcgtctatagctgctggtataatttcagagccaaaatcgttagctgtaggatatctccatttcaaaagcttcaacaatacgtctgtcttgaatacataaactcccattgaagcaatgtagggggatttctttgcatcttgtggagataatccaagaagagtagtatctacttgctgcagcacgaaaaaaatatgagttgaattataaaggaacactagtctgaaagttaaaagtagatgacttggcattactaattgagttttccgttctttttctccccagtcttttatcatacaagagatgtgtctaggttctaactaaacggtcttattccagctatacttgcacgcttgttgatttgtccagattgtacgactaaagattcaattcttttttcaaaattgtaaaggagaaacttttgcaatgaaaatatcctgtatgatacagaggtactgcaacttctcttaactaattttctgaaaatttaaaatgctagccagaagaagaagcaagaaactacttgcgcttaaagcgcaagcttcaaaccagttgagcagtggtttcaatatcacatactgctcaaacaaaaaagtggttgtatatagtaagagttgcaccagaatttgctgcagatcaaagattagaacagctgaataaacaaggcaaaaatctatttaactcggacaggtggaaagaaatcagcacatataacgaaagaaagagtacaactaaacctaccattgctttaagatcaaaaccttttggtttctcagcaaactggacaactctgcctctgctgtcaattttgaccagcccaaaatctgatgctcggctgaaaaaataaagcttaatctcagtttaacccgatcacaggtataaagaaatgtgatgttttcacttcgaagtaatgtatagtgtatctatatgaatattaaaggagaagcattcgtaatgatgaaatatcaatttatcggaatttaaacaaacctgtccccagctagtgcacatgaaagagtaatatcagaattccggtcaatatggttctgtttgaagaacggagaccgttagaaactttctttaaaaaaataaacaaaagaatgaggagcatataggtagttcaagtggacatacctgcaccaactccatataatccatcctataaagatgatccccagataatataaggatattctcaatattcttgttcttagcgtcctgcaagaattagtttccttgtattcacaaaagtactatatatctgtacataataactaattcaggaaaaaataatgaacatttttcagaaattatatcaagtcaatagaagagcagtggtggattaatgtcctaaagagttggagagactaacctcaaaaacccaaataaattgtctaacagcatcagcagttccttgaaaccatctttttccagtttccccaggtgtctgagtagcagctagtacctgttttatgacagaattccttcccgtcagtagaacgaagcaataaagcagaaaaacaaggaagaaatcatcggaagttgttttaatgttacagattttgtgtcaacaaatcacttggaggaccagcaaattcactcaggtatatgaattagcccagctgtgatatctcagcaaacttaaaaaacaaacatggatctaatccaggttcgttgaagaatttcataagtatagtctcaaacctcaacaaatccatctccaaagctcacaccatttccaaaatatgttcgagcaatgtgacgattcaagggagcagaattgtactgtgtcagcacaaaaaccttgttaataccactgttgatacagttactcattgggatgtctattagcttgtagcatcctccaacgggaacctgttgtcatgttgcacaaaatattatgatcgttagatatagaccgcaaagtgaacatcaacttgcaaaccttgtacatactatttctaatcaatctgtctaaggtagaaatcaactaattatataaattatacaaaaaatctgagcacatccaaagaaggaaagtaagaacaagaaatacacacagatgatccaatttcccctaaataagcttcagaacaataaagtgtctactggagaaggggtgagtcatgagggtctgaaccttaacattcatggttggaaaaatcttaaattcaacacaaaagtttggaagaaaaatcagaactttaattggacattagccatctacaacaaaaaaattgagaaaacgaacgtaagttcataagcaaagataaatctcttacagcaggggttgcagctctacttgtgagagggaacaatttggccccttcacctcctcctagtatgactgaagccacatccttgggatttgcccgacgtctctcaaaacgtggcgccgtctcctctaagaactacataggtccagcaaacaatcactatcataggctgataaatattgtagattcaactgaacacaataccttcaacgtgaaacaaatatatagcaaaaaaccagtactactagaacttctaacaaaattaaaactcacaacccgtaatcttaaaagtgaagtatgttcaacgcgaaaaatcttaaatattggacctatcaagcttaaattttggatccgcctccactcacatcacacgtaacaacttctttccaccatatttgcaacactaccatgtcattacttgaggcatacatgtcataaagaagagaaaagaattgatttttaccagagtctcttgttcattttcagtagtgatcacagagaaagcaaccccaggtttgatcttgttctccttcttctcaagtttcaaacttttcgacgactgattaaacttgagattgtttttcaaactccctctgatctgctccccaaaaaactccttctctccattgttaaagccacctttgctcactctccccaaatgggcagtggatttcacagcaacacagcaagtatccatatcagctaata

SEQ ID NO: 50 - DNA, 1227 bp, Capsicum annuum
  source 1..1227: mol_type = genomic DNA
atggatacttgctgtgttgctgtgaaatccactgcccatttggggagagtgagcaaaggtggctttaacaatggagagaaggagttttttggggagcagatcagagggagtttgaaaaacaatctcaagtttaatcagtcgtcgaaaagtttgaaacttgagaagaaggagaacaagatcaaacctggggttgctttctctgtgatcactactgaaaatgaacaagagactctgttcttagaggagacggcgccacgttttgagagacgtcgggcaaatcccaaggatgtggcttcagtcatactaggaggaggtgaaggggccaaattgttccctctcacaagtagagctgcaacccctgctgttcccgttggaggatgctacaagctaatagacatcccaatgagtaactgtatcaacagtggtattaacaaggtttttgtgctgacacagtacaattctgctcccttgaatcgtcacattgctcgaacatattttggaaatggtgtgagctttggagatggatttgttgaggtactagctgctactcagacacctggggaaactggaaaaagatggtttcaaggaactgctgatgctgttagacaatttatttgggtttttgaggacgctaagaacaagaatattgagaatatccttatattatctggggatcatctttataggatggattatatggagttggtgcagaaccatattgaccggaattctgatattactctttcatgtgcactagctggggacagccgagcatcagattttgggctggtcaaaattgacagcagaggcagagttgtccagtttgctgagaaaccaaaaggttttgatcttaaagcaatgcaagtagatactactcttcttggattatctccacaagatgcaaagaaatccccctacattgcttcaatgggagtttatgtattcaagacagacgtattgttgaagcttttgaaatggagatatcctacagctaacgattttggctctgaaattataccagcagctatagacgagcagaatgtccaagcatacatattcagagactactgggaggacattggtacaattaaatctttttatgaggctaacttggagctcacctcagagttcccaaagtttcaattttacgatccaaagacacctttttacacatctccgaggttccttccaccaaccaagatagacaattgcaaggcaagtggataa

SEQ ID NO: 51 - protein, 408 aa, Capsicum annuum
MDTCCVAVKSTAHLGRVSKGGFNNGEKEFFGEQIRGSLKNNLKFNQSSKSLKLEKKENKIKPGVAFSVITTENEQETLFLEETAPRFERRRANPKDVASVILGGGEGAKLFPLTSRAATPAVPVGGCYKLIDIPMSNCINSGINKVFVLTQYNSAPLNRHIARTYFGNGVSFGDGFVEVLAATQTPGETGKRWFQGTADAVRQFIWVFEDAKNKNIENILILSGDHLYRMDYMELVQNHIDRNSDITLSCALAGDSRASDFGLVKIDSRGRVVQFAEKPKGFDLKAMQVDTTLLGLSPQDAKKSPYIASMGVYVFKTDVLLKLLKWRYPTANDFGSEIIPAAIDEQNVQAYIFRDYWEDIGTIKSFYEANLELTSEFPKFQFYDPKTPFYTSPRFLPPTKIDNCKASG

SEQ ID NO: 52 - DNA, 5876 bp, Cucumis sativus
  source 1..5876: mol_type = genomic DNA
ttaaatgagtgaaaaggaatgcacaaacaggttgtcttgaagactttgatttgtaatggaaggtatagaagctgttgaggaagaacactatataggttgcccaaaattctctttatgttccattcaaatccttttaagtaggttatagataaggttctccaattttggtatcctggtgtaggtcggttctgttttagtatgagttttcgtttcaacgtttgctcgtaaattatgttgtaacaaattacattttatgtaaatatcggtttgttagttcatgctatgaaaatgttcaagcaattgatggaactattgttgatgtgatattgtgtaacaatgtagattgtggaatagttctttttcctagtaaaaactataacctaaaagttgtgagatagtgaagtactcttgcaaatttgattgttgtatcgaaaaagttagaatttagtttcatcgattttttaattaattagaatttataactgaattattttttcaacccctagacctcttaaatagcggagagaatcgaaaactaaaaaatataattagatattggtaaaagttaaccgatgtctataaacatcaaattatgttttataaatattttaaatcatttagtcactaacttttggcgtgaattgatatttattgaattaggttcgtttactcgttaaatcctataaagttacagcaacaataaaggatgaccaaagagctgtttttaacattcatacgagagcaattttcgatcatgcacaacatacaacacctctacttaggcctttatgacagtctcatcatcttcttcattctaaaaaaacgaaaaacttgcaaagtataatatatgagaaggtgaattccaaaaaccgtccctataatgatagttgtgattacactctcaaattttcaatggtaaaatttatcttctcaaaagttaaaatcaaactctcaaactcgtgtaattaaactcccaagtaataaaagacaagccttcaaattcatacaaatattacgcaatttctataattatataagtttgagggtccagccataacacttgcaaatatacgatatgagagctcaatttttacgacagaaaatttgaggctatactctatagttgcaattaccactctagcataaggatagctttttgcaatttaccaaaaaaagagaaaaaaaaagtagatggaaaaagggacttatattcatcataccacttatgccccaggaaattgaatttatttatgggttaaaagaaaaggtaatgaaactattcaaagcagaagaggggaggggaggggggggacatttacataatcatcatcataagttaaaagtaacagtggggtatagtgtggagtgtgggtgagggattctcttcctaagctttgccatttactaccaaaagggaatgcatgtggacataatgggtgggattgaacaaggcagacaaaattgagagagagagagagagagagagtggcaccagttgagtgaagtgtgatgtgttgaggtttagatgatagtgccactgggaatcaaagcatccttgataactgtcacaatcccactctttatgaagtaaccatctgtctctcttgctgcttcttgcacattgtctccattcactatctgcgagtatgagattagtattagtattgttattattatcaaccgccgactcacaacatacattatcacacccaacaaaacagttcatatgattacattatcacatccaacaaaacagttcatatgattacaggatcgtgttaatttatttactttaccgacttaacctttctttttcgtttattacttcaactggtttgtttgtttttttttaacaactaggtcgaattttaaaatctaaaaaaaatagttcttaaaaatatattcttgcttttaagtttagaataaaaattgaactcttttacatgatagggatgaaatctataataagaaccgagataaaataagtaaaactttttaaaataaaaaagtatatggttcctattttaaaacttggcttcaatttttgaaaacattagtagaaaatcgataataaaacaagaattttagaggtgtaatgaatggccttaaaaaaaaatctcaagttggcagacactgttacttactttgtagacaatatataatccttgtaattaaaagatttcatatgttaatgtctttaaatctatttaacactcgtgtagacaatatgtaatccttattagacacgttattaaaaattgacacttaaatctaattctcccaaaatatcgaaatagaacacgaaaagggcagaaagcaagtgggcggccatgtggagctgatttaaaacatatttatttccaccgtcaaaaattactttaagagaaaaagaaagctctaatgaactcaggctcttgtctacttgtgtttgttctttcccaatgaagcaatggatttgattgatgattaaacagatgatagaaaccaacacccaaaatgagaccccaccccgaaaacgaatgataagatataataaagacttacttgaagttagcattataaagtaaaagttcttatgaaatataaagaaaagcacaagttgacaagacaaaccttaacattttcaccgatgcgagcgtttttgtcaatgatagctctcttgatgtgagagtttctaccaattccaattggtacacttccctttgccgccaataacctacggtccgcatccgtctgatgaacaccacaagaaaaacaaaccagtttatgctcacaacccatcaatttttcgtttcaactaaccgagtgcaaaacaaacaagacccaatacgcttttcatttcaagatggagatacttggatgcaaccgattaaaggataggtgtagcgtagcctaggatgcacttaagcatttttgctttaggcgagtttcaaacggtaactttactgataaaaagtgatttccaatgtttaactatatagtcaaacaattttgagaatctatctcaaactcgtgataaagaaggttatcaaacctcataataatcagcacccatcagtaacgtatcttctatgattgcaccttcggatatgcaagttcgaatgccaacgacagagtggtgaatcttgcagttctgaggagagaataaaaaaggcgtttattccaaactgaaggaaaggggaagatataatacagtggagacaacgtaacaatggtacaatactaccatcttggcaaagcaaagatctactttactagtaaagaaattacctttatgacacatccctcaccaataacactgtccgtgatatcggcatcaagcattttcgatggtggcaaataacgaggttgggtgtagattggagacgaacgatcatagaagctgcaattggttgtttcgtgtatcaaataaacaaataataacttgacaattttatagggaaaaaaatgaggaaagcgaatcaaacttaacaaaattacctaaaatcaggtactggcttctttgtgatccccagatttgcattgtaaaaagcttcaatggtaccaatatcttcccagtaaccatcatacaagtaagcttgcacctataaagttaaagcaacatcttaattgtagagatcgtaactccaacaaaaactctgaagccatttttctccagcaaacaaacaataatcaaaagtgacacaagtttaagatgagcactctcattcatctgtacttctccttttttcttttatttacttcttatttttgaatcatggtataaagatacatgtaatgagtaaagtttgcatcagacaactagatggttaaggccaccattatttggtacttaccctcattccaatagaagttgcaccaggaattacttcacttccaaaatcatttgctccgggaaatctatctctaagaagattgagcatcacatccttgctgataacgtatatacccatgctagctatgtacggcatctctttggctctctcatcatcaagacccaaaatagtagtatcaacctgtatgaaataaaacatgattgttaagctttgaactaatcatctgaataactattatagttcaccccatggttccatggttggggctctaaatataatagtgacgtttaaaaatatttataaccgacaatcaagtatagtcaatattgtttactattttctactactaaaataatttgaacctcaaacataaaatatcataacccacaaactagaataatcaccactataataaccaactcaacatcccaatcgcctctaaaaaggtatttgagtttggattcaccttcatggctttcaattgctctcctttcggtttctctgcaaactcaattatgcgtccctcttcatcaatcttcatcaaaccaaatgcagttgcacgtttttcatccattggtagagcagctacagtaatatctgcatcggtttccctgtgtgcttgaataaatctctcgtaatccattcgatacaaatgatcaccagcaagaacaagaaactccaacacattctgctcttcaaataaccacaagtactgtctaactgcatcagcagtgccctgcagaatacgattccaaacttaaagaaaatgcatactgaaatcggacgtattaatgttcccagaaacaataatctgaacaaaatggtaggtttttcagcacaattctaatcatgaaagaataaatctatgcttgattccatcacctggaaccaattgggattctctggactctgctgagcggctagaacctcaacaaatccttcattcttgtaaccacccatattactcgcataagctcgggaaagatgacgattaagagaagcagaattgaattgcgtgagaacataaatcttggaaatattgctgttgagacagttgctaacaggaatatcaatcaacctatagttcgctccaagaggaacagcaggcttcgctcgtttcttagtgagcggatagagccgagttccagcaccaccgcccagaataatcccaagcacactctaaacacaacaaaaacagataaaaataaataatccattcaaccaccatcaatttcacaaacaaattcacgtcagctcaaaaacagaaaaccacaggaaataaattcaacgaacgcatcaagaacaccagacaacaaaactacataattccaaaaaattaaacgcattcagataatataacaaagaaacaagaataccaacccggctagcatcgggatcgagacaagtctgagaattcttggaatcagaaaccgctttaggagaaacgatcgagggagccactctacatccagatacacgtcgtgatcccaaaccagaacctctgaggtcaaccttatcaccagaaatgtgagacgcaccgaatgagagactccgaaagctagttctcctcgcacggttagaaccattggacgatgaagacgacgtcggaagtctgagaactccaactgcagccatagaagacgccattagacgatcaattcaaatccaaaatcaacgatatccgataaatcttctccgattagcaagtcaccggaaaaaaaaaatgaaaaagaagaagaagaagaagaagcagagatcagctctgcagctctgcagctctactaccgaataagagaaatgcttcgccactgatatttgatgtggtctcacggacgactaagcccttgtagtttcgactttattacgtggtgtaccgatggaaacgtggtgggggttgtaggggtaatataggaaaaagaaaagtatgaataaaatagaagagattgagtggaggtggattcacggatgtatccatgggtgagtcacatggactcttcccttttagattttttgggaacggtttcattaaaatcaataatt

SEQ ID NO: 53 - DNA, 1566 bp, Cucumis sativus
  source 1..1566: mol_type = genomic DNA
atggcgtcttctatggctgcagttggagttctcagacttccgacgtcgtcttcatcgtccaatggttctaaccgtgcgaggagaactagctttcggagtctctcattcggtgcgtctcacatttctggtgataaggttgacctcagaggttctggtttgggatcacgacgtgtatctggatgtagagtggctccctcgatcgtttctcctaaagcggtttctgattccaagaattctcagacttgtctcgatcccgatgctagccggagtgtgcttgggattattctgggcggtggtgctggaactcggctctatccgctcactaagaaacgagcgaagcctgctgttcctcttggagcgaactataggttgattgatattcctgttagcaactgtctcaacagcaatatttccaagatttatgttctcacgcaattcaattctgcttctcttaatcgtcatctttcccgagcttatgcgagtaatatgggtggttacaagaatgaaggatttgttgaggttctagccgctcagcagagtccagagaatcccaattggttccagggcactgctgatgcagttagacagtacttgtggttatttgaagagcagaatgtgttggagtttcttgttcttgctggtgatcatttgtatcgaatggattacgagagatttattcaagcacacagggaaaccgatgcagatattactgtagctgctctaccaatggatgaaaaacgtgcaactgcatttggtttgatgaagattgatgaagagggacgcataattgagtttgcagagaaaccgaaaggagagcaattgaaagccatgaaggttgatactactattttgggtcttgatgatgagagagccaaagagatgccgtacatagctagcatgggtatatacgttatcagcaaggatgtgatgctcaatcttcttagagatagatttcccggagcaaatgattttggaagtgaagtaattcctggtgcaacttctattggaatgagggtgcaagcttacttgtatgatggttactgggaagatattggtaccattgaagctttttacaatgcaaatctggggatcacaaagaagccagtacctgattttagcttctatgatcgttcgtctccaatctacacccaacctcgttatttgccaccatcgaaaatgcttgatgccgatatcacggacagtgttattggtgagggatgtgtcataaagaactgcaagattcaccactctgtcgttggcattcgaacttgcatatccgaaggtgcaatcatagaagatacgttactgatgggtgctgattattatgagacggatgcggaccgtaggttattggcggcaaagggaagtgtaccaattggaattggtagaaactctcacatcaagagagctatcattgacaaaaacgctcgcatcggtgaaaatgttaagatagtgaatggagacaatgtgcaagaagcagcaagagagacagatggttacttcataaagagtgggattgtgacagttatcaaggatgctttgattcccagtggcactatcatctaa

SEQ ID NO: 54 - protein, 521 aa, Cucumis sativus
MASSMAAVGVLRLPTSSSSSNGSNRARRTSFRSLSFGASHISGDKVDLRGSGLGSRRVSGCRVAPSIVSPKAVSDSKNSQTCLDPDASRSVLGIILGGGAGTRLYPLTKKRAKPAVPLGANYRLIDIPVSNCLNSNISKIYVLTQFNSASLNRHLSRAYASNMGGYKNEGFVEVLAAQQSPENPNWFQGTADAVRQYLWLFEEQNVLEFLVLAGDHLYRMDYERFIQAHRETDADITVAALPMDEKRATAFGLMKIDEEGRIIEFAEKPKGEQLKAMKVDTTILGLDDERAKEMPYIASMGIYVISKDVMLNLLRDRFPGANDFGSEVIPGATSIGMRVQAYLYDGYWEDIGTIEAFYNANLGITKKPVPDFSFYDRSSPIYTQPRYLPPSKMLDADITDSVIGEGCVIKNCKIHHSVVGIRTCISEGAIIEDTLLMGADYYETDADRRLLAAKGSVPIGIGRNSHIKRAIIDKNARIGENVKIVNGDNVQEAARETDGYFIKSGIVTVIKDALIPSGTII

SEQ ID NO: 55 - DNA, 4831 bp, Cucumis sativus
  source 1..4831: mol_type = genomic DNA
tccatggtcaatattggagcttaatattacccgcacaacaattgagcataggtgcgtgcgtgaagaacacccaaataatacattgatttccaataaacaatagaaaatatatatatttccaaaaatattgtagatatttatatatggaatcttccataaattatatacactgcgaggaggaagagagttgagcatgtgaatacagtcgaagatgaagtggacaattcagcaatggacgaaacgaaggcaatccttagaatattctgtcgattcgtaatttgattgatatcaaacgagagggcttaaatctttgatgggcttttccattgatcgccattcacataattggtggttgaaattcattcagcaaagataactttttcccaatccttacttccaactctcatgcactccatgtgaaaacacgcctttctcaattacgtaactttccattctccatttctcacaatggccgtctcctccgactgccggattgccctctcggcggccgcacctgccacttcactggtggtccggaggaggttgacgtctgtggagtttggcaatggggaattcatggggaagaaactcacggaacccaaacgagtacctgctgctgctgttgctattaacggtgttcggcgacgtgtttgcatgtctctcacgactaatgtagccattggcgatgctgcggtaatgtttagaactctgcagatatttgtgttttttggttttcttgtgttgtggaccttgaaatgccttaggtttatgaaactacgacttctttgttgattcacattgatttctgatgattcacattgatttctgagagtatgtttgttaacatgttggaccgttggtgatgatctgtctctgtttttcgatagaattttctctgtagacgttgccaactactatggcatttactcatttgaatttttgtttattaccctacgccttctggttgttccagttgaatctatgaacttggtgcaatttggacttagtctgatatgtatatatacgaacaaacctgagatatacaattgggaatggaaacaagggactgaaaaaccgttagagcttcataaatctttgtgaggcctattcagaaatattgatctgaggcaataatgaatgtcttgttgcttgtgactttgtctagttgagagaaatggatatggagaaaagggacccccggactgtggtggcgatcattctggggggaggagccggcactcgtctattccccctcaccaagcggcgggccaaacctgctgtaagtttcctctactgaactcaatttttcttatagtgatagagatcatttatggttctttcagtttagatgattggaatttaatgacattttattttcatatatatatgcatatgggattttgctacaatggctgctctgtgatttcttatgatttaccattacaggttccaataggaggagcatataggctgatcgacgtgccaatgagcaactgtatcaatagtggaattaataaagtctatattctcactcagttcaattcagcatcattaaataggcatcttgctcgggcttacaacttcagcggtggagtcacctttggagatggctatgttgaggtacgtttcattcacataaaaaatgtcccttaagaattaagtgaatcatatatccacaattatattccccaaaccaaatatgacttatagagctttcagctatatcttaggttcttgctgccactcaaacgccaggggaggctggtaaaaattggtttcaaggtacagcagatgccgtgcgacaattccactggctttttgaggtaccaaaagataaaaaaaaggaaaatttttatctaatagcatagtaagcaaattattgtgttgagaagaatataaagaaacgattgttcatgacattttagctgttccatatacaatattcagcataaagataaaacaaagtgacgaaaggaaatatcaaatgttaaatagagtgataagcctttcctggtgcaggatgccagaagcaagaatattgaagacgtgttgattctgtctggagatcatttgtaccgaatggactatatggattttgttcaggtaaggctgaagatgccatcaagaaccaatctttatttgttattacgtttagttctgatacagcttctttacattggcagaatcataggcaaagtggtgcagatatcactctttcttgtctgccaatggacgataggtgagtgacatcttgatgttttcttctgaacaattaacttaaaaattattgtattaaactaccgattgaccaaaagcttaagctcatggtttatggtaaatttcattgcccctcacttaggtaggaaatgacattgtaagagtttgaatatgagactttcttctttgataccatataaaatcaccaattgacccaaaagcttaaactcatggattatgttaaatataattatactacttttaacacgttgatctcgaaaagccccagagatcttgatcatgcaatcagttcaaacctttttacgtttgtagaaacttgagtatctaggtctaaaggttgaaattattacagaaatatgtaagaaaggagaagcatttttacttcaaatttatgtttcttttaattgagtatctaatactgctgcaatttatttacaaaacgcattaccttttattgtagccgagcctcagactttggtctaatgaagatagacgaaaaaggaaggataatttcattcagtgaaaagcctaaaggacaggacctgaaggccatggtaacattcaaaattgctttcaccacaaaattaaatcggtggaggaactttaaatgatcttgctgatgagcctaaactgtatcatctctattcgaattgaaggcagtagatacaaccattttaggactctcgaaagaagaggctcagaagaagccatacattgcttccatgggagtttatgtctttaagaaggacatacttttgaatcttttaaggtattatgaacctaatgctatttatcaatctagtaattttatacaaactcaaaggactttgtcgaacacaaaatattagacgttgcttgaatttgttcaagtcagatggcgctttccaactgctaatgactttggatcagagattatccctgcctcagccaaagaattctttataaaggtttgtaatttgtatgtaacctttctgtcgagactgttatatcatctttttcaactttctacttgcaatttagacacaaactaatgctaacctcaacaaattttcacaggcttatctcttcaatgattactgggaagatattgggacaatcagatccttctttgaagcgaaccttgccctgacagagcaagtaagacaactaaacattttttatttacaatttcttaccctctctcaccctttagtacttcctcaaattactagaattgtaatttgatttttctttttatcttgctaatcttgctgttcaatctcatgtttgcttatacctttcagccaccaaggtttagtttttacgatgctgcaaaaccaatgtttacttcaagaagaaacttgccgcctacaaagattgaccagagcaaggtcagtagcattttgcagtcatctaaacctctattataaaatcataaactttaacaaaaccttcattcttgtttaacgaaatatgctgtagactgccttaaaataacactgcatttggcataattttctttgcttgttatgttgagaaaatatctgggttctttgcagatcgtagattctatcatctcacatggaagcttcttaaacagttgctttatagagcacagtgtcatcggtatccggtcgagaatcaactcaaacgttcacttgaaagtatgtactttatggaacttttgcatgatatggtaaacatatcagatttctgtaacatgatggttaccatagaactgagtttcttaccaaagaaaaaacgaactgagtccaatccaatcctttgcaaaacgaaagtgtttttcttaaacaatgctgtgtgatgagttgcaaaatgcaggacactgtgatgcttggagcagattactatgaaactgaagacgaagttgcatctttgttagctgaaggaagagtaccaataggaataggggaaaatacaaaaataaagtaagaaccccatcacacccaaaaaaggatcttcatatgttatgtagcctgctttctaattcttcattgttgatataaatattatgtgcagggactgcattatcgacaagaatgctagaatcggaaagaacgtggtcattgccaattctgaggtaccgttaccatacgaatagatttgaaatgaggttggaatgaataaatttatgttttcattggaatcgtattgcagggcgtacaagaggcggatagatcttcagaaggcttctacatacgttctggcgttaccatcatcttaaagaatgcagtaattaaagatggtttggttatataataaaacatgtttgccaatcgttcattgatttgcattatttctttagttatttttaattttctgtatcacataaaataagaaagcagaggatgtaagctaacagccaaatatatctatggatgtcattcttcaacgcaacgactatcccaagtatgtttttatttggccaacttgctttgaaggaaaaaaaaccatttttttctcgtgtcatcatccttaggtcctaaaggaccattgctttagtgcacttccagcatcagaaaatacaagtctaaacaactagctcataaaaaaatgtatactcagttggttgtccatttgatttttgaatatt

SEQ ID NO: 56 - DNA, 1569 bp, Cucumis sativus
  source 1..1569: mol_type = genomic DNA
atggccgtctcctccgactgccggattgccctctcggcggccgcacctgccacttcactggtggtccggaggaggttgacgtctgtggagtttggcaatggggaattcatggggaagaaactcacggaacccaaacgagtacctgctgctgctgttgctattaacggtgttcggcgacgtgtttgcatgtctctcacgactaatgtagccattggcgatgctgcgttgagagaaatggatatggagaaaagggacccccggactgtggtggcgatcattctggggggaggagccggcactcgtctattccccctcaccaagcggcgggccaaacctgctgttccaataggaggagcatataggctgatcgacgtgccaatgagcaactgtatcaatagtggaattaataaagtctatattctcactcagttcaattcagcatcattaaataggcatcttgctcgggcttacaacttcagcggtggagtcacctttggagatggctatgttgaggttcttgctgccactcaaacgccaggggaggctggtaaaaattggtttcaaggtacagcagatgccgtgcgacaattccactggctttttgaggatgccagaagcaagaatattgaagacgtgttgattctgtctggagatcatttgtaccgaatggactatatggattttgttcagaatcataggcaaagtggtgcagatatcactctttcttgtctgccaatggacgatagccgagcctcagactttggtctaatgaagatagacgaaaaaggaaggataatttcattcagtgaaaagcctaaaggacaggacctgaaggccatggcagtagatacaaccattttaggactctcgaaagaagaggctcagaagaagccatacattgcttccatgggagtttatgtctttaagaaggacatacttttgaatcttttaagatggcgctttccaactgctaatgactttggatcagagattatccctgcctcagccaaagaattctttataaaggcttatctcttcaatgattactgggaagatattgggacaatcagatccttctttgaagcgaaccttgccctgacagagcaaccaccaaggtttagtttttacgatgctgcaaaaccaatgtttacttcaagaagaaacttgccgcctacaaagattgaccagagcaagatcgtagattctatcatctcacatggaagcttcttaaacagttgctttatagagcacagtgtcatcggtatccggtcgagaatcaactcaaacgttcacttgaaagacactgtgatgcttggagcagattactatgaaactgaagacgaagttgcatctttgttagctgaaggaagagtaccaataggaataggggaaaatacaaaaataaaggactgcattatcgacaagaatgctagaatcggaaagaacgtggtcattgccaattctgagggcgtacaagaggcggatagatcttcagaaggcttctacatacgttctggcgttaccatcatcttaaagaatgcagtaattaaagatggtttggttatataa

SEQ ID NO: 57 - protein, 522 aa, Cucumis sativus
MAVSSDCRIALSAAAPATSLVVRRRLTSVEFGNGEFMGKKLTEPKRVPAAAVAINGVRRRVCMSLTTNVAIGDAALREMDMEKRDPRTVVAIILGGGAGTRLFPLTKRRAKPAVPIGGAYRLIDVPMSNCINSGINKVYILTQFNSASLNRHLARAYNFSGGVTFGDGYVEVLAATQTPGEAGKNWFQGTADAVRQFHWLFEDARSKNIEDVLILSGDHLYRMDYMDFVQNHRQSGADITLSCLPMDDSRASDFGLMKIDEKGRIISFSEKPKGQDLKAMAVDTTILGLSKEEAQKKPYIASMGVYVFKKDILLNLLRWRFPTANDFGSEIIPASAKEFFIKAYLFNDYWEDIGTIRSFFEANLALTEQPPRFSFYDAAKPMFTSRRNLPPTKIDQSKIVDSIISHGSFLNSCFIEHSVIGIRSRINSNVHLKDTVMLGADYYETEDEVASLLAEGRVPIGIGENTKIKDCIIDKNARIGKNVVIANSEGVQEADRSSEGFYIRSGVTIILKNAVIKDGLVI

SEQ ID NO: 58 - DNA, 4446 bp, Cucumis sativus
  source 1..4446: mol_type = genomic DNA
aatagaaatgctaagcaaatattgacaacttaatataacttgagttttaagagagtaactctttcacagagatgagatttgctcgtccagaagctaatgtcaaatggctttgaaaactgttccctaacgatgtatggagttgttattactcaggatggaaatgtgctgcctttctactttttacatatatgaaaagaaactagtattttacatcaagggaagttgcactattactcgttgccgagcattgtaggttgaaagccttgagcaattgcttcctttcaactctcagctatctccagacctgaatgagaggctgccattggattttaaataatggttccatcttttattgttgcattcttcaacgtgactgtgataccggaccttatataaaacccttcttctggtctttctgcttcttgaacgtcctgaaaagttaataagtacactttcttaagcataggcactatgatcaaataacattacgcgcttatttggtttaaattgaggttgcaaattcttagaaagtgttcaacttttgggctaaacttatgtaacttgtgcttaaaaagtcaaaattgatgtttgataaacatgtgccttttctcatcccaattgttaatcctaccaattacatggactcaaatgatatattgttcatcctaccaaacctaagtatttattgtttatttctttgcacatattaatcataaataaaacagtttgaaccttgtaaattcaacgtatttaaattgttttgcaataaccaatttaaaactattggccaaacatcccttgcaataatagattgcagatagcttttgctctttatttgcaataaccacatttcttcaagaacagaaattatacttacatcagtatttgcaataaccacatttcttccgatcttggcattcttgtcaattatgcaattcctgcagggaagttacaaaacatcacaaacgaaaccaaaccctaatttgtatgttaatattaaattaataaacgattgcacatagcttttgctctttattggtagctgaatattacctgattttggtgttctcgccgatgccaattgggatctttccttcagcaaggagagatgcaatttcagattctgtctggtagtagtccgcacccatcatcatagtatcctataatgcaaaaccaacaaattcaaatatgattttagcttttttcaagagatgtggcaacagctagtaacaaacatttattacgtattcaatttggattgatccatttactattataagtcagcatgacggactctctaacacaattaaaaagagtttaatctgtggaaaaaaaattgcagcatctacaaagctagcttctgcaactgcatttagccttcttgtactcccctatggtataagacgaaaatccaaaagaaaactatggctcaccttaagctccacaccatactccagtcgggagcgcacgccaacaatagagtgttcaacagtacactctcggaggaagcaaccatgggatataattgcatctacaatctgcatacagcttcatttaagacacaagaataaagcagagagagaaaacttgagtttgatgtcttaccttgcatttttcaactttagaagggggcaagaatctaggtgaggtataaaatggagtcttgggatcataaaactcaaattttgggggctgcatgaaagacacaacaaaccatcagcttgtgtaaataatagtgtttggagaaagaccttgcttgattacacttgatcatctgttttttctgattagtgggaggggttgggttgaaattttcacagtgccgttaaggtacttgatattgattaagggtggtagtacttaaggagaatatgagcagaagtaacatgagagcttatcatctatgtaaatatctaacttaatgaacaaataagcatgaaagccgaaagaagtggtgtaagatgtttcttgatcccataaaatgaaaaagaaagaagaccatcgaaggcctgagacacaattgcttgtatgatgggatacatacctgctccgttagtgctaagttggcatcaaaaaatgactttactgttccaatgtcctcccagtagtcattgaacagatatgcctacaacaatatgaatgcattgggaaacttagttgtggtattgtcgaaagtcttccttttctgaactcaagcatataaaggaaaatgatagactaaaatttctcaaaacggatctcacttgaactttgtagtctttaacagccgcaggaataatttctgagccaaagtcattgcacgcaggataactccatgtcaaaagctttaatagaagatctgttctgaatacgtagactcccattgatgcaatataaggattctttctagcatcttcatctgaaagtcctagaaccgtagtgtcaacttgctgcagtacaatatatgacgatataaaacaaaatcagacatcgatacaggtcactgtaaggaacacgagtcaataaatacatttacgctaaaaaactaaccaaacaaataaaaaagagaaaaaacaaaagaagttcaagaaaaataactaattgcatcaatgctaccaaccattgcttccaggtcagatccctttggtttctctgcaaaatctaaaatacgtcctgtatcatctattttcatgagaccgtagtctgatgctcggctgcataacagcaaaacatgaatgaaaatttaaatttttttaaggaaataaagaacaagattatggtttccatgcactttatctatgaaaataaggattaaaatggatgcctacctatcatccatgggtatgcatgaaacagtaatatctgcatttgtgtcaatatgtctctgcaagtacaaagccatcggaaagtctcattgactcacaaacaaatatgcaaaatactacaaatacaattacaaactagtcattttaacattatacctgcacaaaatccatgtagtccatccggtaaaggtgatcgccagacaaaattagagtgtgctcaacatttttggtcttggcatcctgtacaaacccaatggacaaatgttcattacaattaaaaagttcactgaaaattctaatcatactcagtagattttatgcacccacctcaaataaccatataaattgcctgacagcatcagcagttccttggaaccatttttttccagtttcacccgaagtctgagtggcagctagaacctatttctcaaacatgaatccagagattgagtataagaaagcttcctgtttcctagaacttgccgtctgtctaattaacacaattactaggcagggtcagattgcaacaatgagaagaacattcatcatacctccacgaatccatcgccaaaattgactccattaccaaaattataaatacgagcaaggtgacgatttagagaaaatgagttaaactgcgttaagacgaatatcttttcgatgccactattgatgcagttactcatgggtatgtcgatcaacctataacaacctccaattggaacctgcaagcaaatattatttacttaaattcgtgcactaactctttctcctagattagatcaatagcttatgttatctactacggaatcatccttctcttagcaatttggtagagttggttaccgctggtttggctcgctgactggtaagaggaaacaaacgagttccagcacctccacccagtataatcgaagcaatctttttcggatcagctcttggagcttcaaatataggagcctgtaaagtctgagagataaaatttgaaataaaaaatcatgtaaaagtttatatacttttgaacaacaaagcacaaaacccatttaccgatgtttcctcattaatttctgacatgagaacagaataagcaacaccgggtgcaagcttcctagccctgctaaaagaagaagaattagaattcttgagcgagtaggcattcctctgtatctgtttggaccttccgtttcttccaataccgtctccccaaaacccagagcattgattcctaacacactgacttgtgatcctaaatggcatcgtattgggcttcaaggccacaccaaaggaatccattgttgaaatgaagtgacccgaaaacggggtagatcagtgggaaacctaagtaagaagaaggtgtgagagaaatgtagaatgaagagagtgggagggaagaagaaaggcgggagtgggagaaagatgaaagatcaaatgatggtgatggtgacgaaaggtatggaaaatagcgtcattaaattatttggggttggggtgccatggaaaagtctggggatcttgatttttttcatgagagagccggtctgggagttgaacacggcggcataagactgattgcaaatggtatgggaccaaacctttttcagcttctctcttggcctctactactcatctctttctaacaaat

SEQ ID NO: 59 - DNA, 1599 bp, Cucumis sativus
  source 1..1599: mol_type = genomic DNA
atggattcctttggtgtggccttgaagcccaatacgatgccatttaggatcacaagtcagtgtgttaggaatcaatgctctgggttttggggagacggtattggaagaaacggaaggtccaaacagatacagaggaatgcctactcgctcaagaattctaattcttcttcttttagcagggctaggaagcttgcacccggtgttgcttattctgttctcatgtcagaaattaatgaggaaacatcgactttacaggctcctatatttgaagctccaagagctgatccgaaaaagattgcttcgattatactgggtggaggtgctggaactcgtttgtttcctcttaccagtcagcgagccaaaccagcggttccaattggaggttgttataggttgatcgacatacccatgagtaactgcatcaatagtggcatcgaaaagatattcgtcttaacgcagtttaactcattttctctaaatcgtcaccttgctcgtatttataattttggtaatggagtcaattttggcgatggattcgtggaggttctagctgccactcagacttcgggtgaaactggaaaaaaatggttccaaggaactgctgatgctgtcaggcaatttatatggttatttgaggatgccaagaccaaaaatgttgagcacactctaattttgtctggcgatcacctttaccggatggactacatggattttgtgcagagacatattgacacaaatgcagatattactgtttcatgcatacccatggatgatagccgagcatcagactacggtctcatgaaaatagatgatacaggacgtattttagattttgcagagaaaccaaagggatctgacctggaagcaatgcaagttgacactacggttctaggactttcagatgaagatgctagaaagaatccttatattgcatcaatgggagtctacgtattcagaacagatcttctattaaagcttttgacatggagttatcctgcgtgcaatgactttggctcagaaattattcctgcggctgttaaagactacaaagttcaagcatatctgttcaatgactactgggaggacattggaacagtaaagtcattttttgatgccaacttagcactaacggagcagcccccaaaatttgagttttatgatcccaagactccattttatacctcacctagattcttgcccccttctaaagttgaaaaatgcaagattgtagatgcaattatatcccatggttgcttcctccgagagtgtactgttgaacactctattgttggcgtgcgctcccgactggagtatggtgtggagcttaaggatactatgatgatgggtgcggactactaccagacagaatctgaaattgcatctctccttgctgaaggaaagatcccaattggcatcggcgagaacaccaaaatcaggaattgcataattgacaagaatgccaagatcggaagaaatgtggttattgcaaatactgatgacgttcaagaagcagaaagaccagaagaagggttttatataaggtccggtatcacagtcacgttgaagaatgcaacaataaaagatggaaccattatttaa

SEQ ID NO: 60 - protein, 532 aa, Cucumis sativus
MDSFGVALKPNTMPFRITSQCVRNQCSGFWGDGIGRNGRSKQIQRNAYSLKNSNSSSFSRARKLAPGVAYSVLMSEINEETSTLQAPIFEAPRADPKKIASIILGGGAGTRLFPLTSQRAKPAVPIGGCYRLIDIPMSNCINSGIEKIFVLTQFNSFSLNRHLARIYNFGNGVNFGDGFVEVLAATQTSGETGKKWFQGTADAVRQFIWLFEDAKTKNVEHTLILSGDHLYRMDYMDFVQRHIDTNADITVSCIPMDDSRASDYGLMKIDDTGRILDFAEKPKGSDLEAMQVDTTVLGLSDEDARKNPYIASMGVYVFRTDLLLKLLTWSYPACNDFGSEIIPAAVKDYKVQAYLFNDYWEDIGTVKSFFDANLALTEQPPKFEFYDPKTPFYTSPRFLPPSKVEKCKIVDAIISHGCFLRECTVEHSIVGVRSRLEYGVELKDTMMMGADYYQTESEIASLLAEGKIPIGIGENTKIRNCIIDKNAKIGRNVVIANTDDVQEAERPEEGFYIRSGITVTLKNATIKDGTII

SEQ ID NO: 61 - DNA, 5262 bp, Cucumis sativus
  source 1..5262: mol_type = genomic DNA
gtttactaactaatgtgttcggcgatcattcttcaatattgtttcatcgaaattttcatatttgtatttgcactaaattatatctttgcacatgttgacaaaagtatcaggtaaaaaaagtgaatcccattataataacaaaatgtgaaatatatgaattcagatgccattaatcaaagatcttgatgttgattacaatttgaacactctcccaactcttgaaacttggaagtgcaccaaataccaatcaacttatagaagtaaatgattatggataatacagcaacataacaattcctatgtacataacataaccttactaatctggctgtgtgtagccaattcttattgaacagaattacactaaagatttagaatctcttccccatgtggaacttccacttcatcaaacctcttaaaagctatctctaaagattctgaaaagaagctttaaactcaatctccatactcaaatcctttctcctttttctcagctacaattgctgattcttcagctgccacagtttaggcagtctcatgaaaaccaacttcacaccattttcctccctcctctaccatgtcttcaaaaccaagatttaggattctgagttctgttcatataacggtgccatctccgactgttgccttttccattataatggtgattcccgaccgaatgtagaatccctgttcaggcctagctgcttcttggacgccctggaaattgatagagaaacagattaatggaaagagactaatgaggttttcaagactgaatgtctattgttaaggatgggctgagatatacttacatctttgttcataatgataacatcctttccgatttttgcattcttatcaattatacatttcctgcaacaaaccacacccataatgataactcaatattgttggattggaaaattctcacgcagttctatgtttaacaagtttctctcaaggttcgagtctacttacttgattttcgagtttcgtccaatccctacagggacttttccctctgctagcagacctgtaatttcaagttcggtttggtaattgtctgcacccatcattattgtatcctgtgtaaaagaagatagcaacgacggttacaaatccaagtgtttgtccaacaacctgtcttaaactatgagttcttcgctttcaccttaagttcaacaccgtagtctaatcttgaccgttcaccgactattgaatgttggacactacaatctctcaaaaagcatccatgtgagattattgcatcgacaatctgcagaactcaaacgaagcttaagagcagattcctaaaaacaagcttagaatcagattggttttattcatatacctgacatctgtcgatcttggtcggcggtaagaaccggggagacgtgtagaacggtgtcttggggtcataaaattcaaacttaggaaactgcaagaaaatgaaaaattgataatcaataccaactgaaatgaagcaattggcaatgccaggtaagttctagaaaatgaatcctttcagcagagaatgcattgcttgaagcagaggactttgaccttcagttaattccacagttcagattcttaagataacgattgcaaagtcaagtaataaactatatggacagattatacaactataaaggaaaatcagaacgaaaaaaatttgagtccaagaaatgtaacgtacctcttccgtgagggccatgtttgcatcatagaaagtctttattgttccaatgtcctcccaataatctctaaacataaatgccttcagaataagaaaacaaggaagaagaaagaaacaaaagttaaacgcacacattatacatcaatgtgaaaacaatgtctcaaatgaagttgaaaatatgagggattgagataggctatgatagaagagaaatggaagtcaaaataggagttatcttttactagttacagcgaaaaacaaaggcctctagcacacaaaattgattgtaggcgaacataatttatgcagcattttgtatcaagaaccattacgtaatcacacaaatataaaattttaccagttaaagattcaagaacagagtaggtcgacacaattattaccttgacaaatttaattaataaagcattaaggagttgatagaaaatcatcttacttggacattgtattcctttatagctgcaggaatgatttcagagccgaagtcattagatgtaggatatctccacttcaaaaggtttagtaaaacctctgttttgaaaacataaactcccattgatgcaatgtaaggagacttcaatgactcctcccgagacagaccaaatggagttgtatccactcgctgcagaataccaaacaattgggatgattatatcacttgaacaagactcaaattacaagataaaattttgatgcttaaattaggaatgtaatttgagaaacaatgattgaagagaaggttcagtaaacatgaaccaatttcagccaagaatgatggctttaatggctttcacgacttaagccaatatagttcattcacaaagtggaaaaagtaaccattctatgttttctgcagtttttaccattgcattcaaattggcaccctctggcttttcagaaaactggataattcgacctctactatctaatttcaccaatccgtagtctgatgcgcggctgcaaagtggattaaaaggatatgagactttgactctaaaatcctaaagaacaaataaataaattcctatgatctagaagcatttcaatttcgttgttctttaagcatacaaaataaggttttgaagttaacttagacaaacaattgcgcttatcaaatcatttactatttacctacaaccaaaacaattttaaacgaagaattataaggtaaaaggaacattgataaaacctgtcatccacagctgcacacgagattgaaatatcagctttgcgatcaatgtgattctgtgtccaatgatagcatacaattagattttgaaatatagaatatgaactattatctttatcagaatggggcttcaaaaagaaattcattacctgaacaaagtccatatagtccattctgtacatgtgatcccctgccaaaattagaatgttctcaacatttctgtgcttggcatcctacaagaaataatacgaaaatcaacttcataatatcaacatggtgaagtttcaaacagaacaaaaagaaaataaagttgttttatacctcaaatacccaaataaattgtctcacagcatctgcagttccttggaaccagtacataccagattccccagatgtttgtgtagctgcaagaacctgtttatcgagagtaagctcaaggtcatattataatctagagatctggaataactaactactgaaaataatcacaattatacactctcattttccaaaataaaacagtcaagttaggaaaaaaaaattgactctatctctaagttgtcttttttattccatgctttttatcattaagttcttcaattatcgattcaagcatttttcaatagcttcctacacattgccataccatacctccacaaatccttctccaaaagtgacaccatttccaaagtatgtccgtgcgatatgacgattcaaagaagcagagttgaactgtgtaagcacaaatattttgttgatcccactgttgatgcagttgctcattggaatatctataagcctatagcatcctccaactggaacctacccatgcaatgcaagcaaatatttgataggtaaatctggcaaaacatacaagacccgatggcaaataatctggactatatgacacaccataaaaaaaaaatgacaaagatattaggaaaaccttccttcaatacatgatttggaaagaaaaaaaaaacagattcatatattctaagaaatacattaacatcagtgagaacaaaattacattttctttggtgtaaaaaacagagaactgattggaaaacgatctaaactaagccgctgacccttgtaaagttccacaatcaaatatatatatacacacagacaaattccttacagcgggtgttgctgatcttttagtaaggggaaacaggtgagtcccagcaccccctcccaatatgattgaagcaacatttttgggattcgcttttactttaggaactgttggaacttgaatggtctgcaaaatcaaatccaattcaaagaaatgaaaaatgaagaaaaatcatgatggggttttcatcaaaacttccaaagatataaatcaaacatggccatttggccatccacaaatgcagtgaactcaccataggctgctttgaaacattaggcgtagccacagcataagtaacatttggggtaagcttcaaagctttcttctcagatttcaaactcctaatccaaacattttcactaaaacctcctctaactttctcaccaaaaaatctattctcacaacgatcaaaacctccccaattacccttcatcaattgggtatcggatttcaaagacacaaagcaggaatccatcgcagccatatctcactagcaataccaaatccaaaaaatcagttaactctctcgaataaacaatgcattagaaaatgtcacaaattacaatcaaatagcattccccccagctcaaatcccaatagaaaaagcaaaagccacaatccaagcaaccaaatagagtacagatcacacaaaggaaagaaaaaaagtaaaaaggtagctaaaaaatggaagaaatcacctgaaaattctgaaagacagtaaaatgagagagacccagataataaaatgatgaaaataagaaaattgagaaagggaaaggagaataaatggaaaactcagaaagaggagacggatcaaaagcagagagaaaggggaaaaaagagtgaaaaatgtgagtgatgacgaacaaggcggtgggttatgggcatgtgaatgtaaggcgttttcattcacgaggattgatttttataggaaaaaacaagaaagtgggaaagcttcattcattcatgtcagcaaatgcttgttctttgttcttcatcaccttcttcataattattaaaagaaaaaagaagaagattattttggtacaaaactcaatttgcaacagatcatccgctatttgtacgccgcattctacttggacccatgtatttgtgtacaaattcaaaattatttcagagataaaatagaatatggaaattgatggatagagtaaaactcttaattaatacataaaatttagaaagggtaaggta

SEQ ID NO: 62 - DNA, 1581 bp, Cucumis sativus
  source 1..1581: mol_type = genomic DNA
atggctgcgatggattcctgctttgtgtctttgaaatccgatacccaattgatgaagggtaattggggaggttttgatcgttgtgagaatagattttttggtgagaaagttagaggaggttttagtgaaaatgtttggattaggagtttgaaatctgagaagaaagctttgaagcttaccccaaatgttacttatgctgtggctacgcctaatgtttcaaagcagcctatgaccattcaagttccaacagttcctaaagtaaaagcgaatcccaaaaatgttgcttcaatcatattgggagggggtgctgggactcacctgtttccccttactaaaagatcagcaacacccgctgttccagttggaggatgctataggcttatagatattccaatgagcaactgcatcaacagtgggatcaacaaaatatttgtgcttacacagttcaactctgcttctttgaatcgtcatatcgcacggacatactttggaaatggtgtcacttttggagaaggatttgtggaggttcttgcagctacacaaacatctggggaatctggtatgtactggttccaaggaactgcagatgctgtgagacaatttatttgggtatttgaggatgccaagcacagaaatgttgagaacattctaattttggcaggggatcacatgtacagaatggactatatggactttgttcagaatcacattgatcgcaaagctgatatttcaatctcgtgtgcagctgtggatgacagccgcgcatcagactacggattggtgaaattagatagtagaggtcgaattatccagttttctgaaaagccagagggtgccaatttgaatgcaatgcgagtggatacaactccatttggtctgtctcgggaggagtcattgaagtctccttacattgcatcaatgggagtttatgttttcaaaacagaggttttactaaaccttttgaagtggagatatcctacatctaatgacttcggctctgaaatcattcctgcagctataaaggaatacaatgtccaagcatttatgtttagagattattgggaggacattggaacaataaagactttctatgatgcaaacatggccctcacggaagagtttcctaagtttgaattttatgaccccaagacaccgttctacacgtctccccggttcttaccgccgaccaagatcgacagatgtcagattgtcgatgcaataatctcacatggatgctttttgagagattgtagtgtccaacattcaatagtcggtgaacggtcaagattagactacggtgttgaacttaaggatacaataatgatgggtgcagacaattaccaaaccgaacttgaaattacaggtctgctagcagagggaaaagtccctgtagggattggacgaaactcgaaaatcaagaaatgtataattgataagaatgcaaaaatcggaaaggatgttatcattatgaacaaagatggcgtccaagaagcagctaggcctgaacagggattctacattcggtcgggaatcaccattataatggaaaaggcaacagtcggagatggcaccgttatatga

SEQ ID NO: 63 - protein, 526 aa, Cucumis sativus
MAAMDSCFVSLKSDTQLMKGNWGGFDRCENRFFGEKVRGGFSENVWIRSLKSEKKALKLTPNVTYAVATPNVSKQPMTIQVPTVPKVKANPKNVASIILGGGAGTHLFPLTKRSATPAVPVGGCYRLIDIPMSNCINSGINKIFVLTQFNSASLNRHIARTYFGNGVTFGEGFVEVLAATQTSGESGMYWFQGTADAVRQFIWVFEDAKHRNVENILILAGDHMYRMDYMDFVQNHIDRKADISISCAAVDDSRASDYGLVKLDSRGRIIQFSEKPEGANLNAMRVDTTPFGLSREESLKSPYIASMGVYVFKTEVLLNLLKWRYPTSNDFGSEIIPAAIKEYNVQAFMFRDYWEDIGTIKTFYDANMALTEEFPKFEFYDPKTPFYTSPRFLPPTKIDRCQIVDAIISHGCFLRDCSVQHSIVGERSRLDYGVELKDTIMMGADNYQTELEITGLLAEGKVPVGIGRNSKIKKCIIDKNAKIGKDVIIMNKDGVQEAARPEQGFYIRSGITIIMEKATVGDGTVI

SEQ ID NO: 64 - DNA, 4169 bp, Cucurbita maxima
  source 1..4169: mol_type = genomic DNA
gggtaaaatcgacactgagaccacatcaaatatcagtggcaaatcggttccctcattcggcagttgagctgcgagctaatctctagtattttcgtttttcccggccactgtctgatcggtgaagaaaccggatttagctttgtgaattcggattgatcgtccaatggcgtcttctattgctgcaggtggagttctcagagtttcgacgacgtcggggtcttcgtcgtcgaacggcggttctaaccgtgtaaggggaagcagcgttcgaagcctctcgttcggtgcgtctctcatttctggtgacaagattgattttagagcttctgctttgggatcacgacgtttttctggtggtagagcggcacctttgatcgtctctccgaaagcggtttctgattccaagaactctcagacttgcctcgatcccgatgctagccgggttggtattcttgtttctttgtgttgttctctgaatgcgttaattgattggaatcgaatagttttagagcctgttgtacttgatgcgttcgcggagtttatttcgtttgatttcctgtattgagccgctgcgatttgtttctagaatggatggtgatttaatggcgtctgttatctgttttttttttgtggttagagtgtgcttgggattattctgggaggtggtgctggaacgcggctctatccgctcactaagaagcgggcgaagcctgctgttcctcttggagcaaactacaggctgattgacattcctgttagcaattgtctcaacagcaacatttccaagatttatgtcctaacgcaattcaattctgcgtctcttaatcgtcatctttcccgagcttatgcgagtaacatgggcggttacaagaatgaaggattcgttgaggttcttgcggctcagcagagtccggagaatccaaattggttccaggtgatggaatcaagattaggtttattctttcataattcacgcgaaaattggaatcactgaaacctaccattttgttcagaacattgatcttgtgatatactctgtcttaatacttccaatttcctaatgcattttctttaagtttgtcttgatctgatatactccgtcttatctcataaatgtttggaattgaaacctgcagggaacagctgatgcggttaggcagtatttatggctatttgaagaacacaatgttttggagtatctagttcttgctggcgatcatttgtaccgaatggactacgagagatttattcaagcacacagggagactgatgcagatatcactgtagctgcacttcccatggatgaaagccgtgctactgcatttggtctgatgaaaattgatgaagagggacggataattgagtttgcagagaaaccgaaaggagaccaattgaaagcaatgcaggtgaatccaaactccaatactttttatcataataaactgttctcctatgtcttgcttcataagatgttcttgttagatgggttagtctaaagcagaaaaaaaaatcctgttttctttcttacaggttgatactactattttgggtcttgatgatgagagagcgaaagagatgcctttcatagccagcatgggtatttacgttatcagtaaggatgtgatgctgaatcttctaagagaaaaatttcccggagcaaatgattttgggagtgaagttattcctggtgcaacttccattggaatgagggtatggatataatgatcttatgcaccataaatatatgtcatatcaatattgatgaaagtattgtccaaatatatcatagaattcgttagtggaaagtaccaaacaacgatggccttgacaatcgagttgtctgatacaaactctagccactacatgtgtaagatcttacatctgttggggaggagaacgaaacattctttataagggtgtgaaaacctttcactagtagactcatttaataatcttaagggaaagccccaaaagagaaaacctaaagaggacattatgtgctagtggtgggcttgagctgatctgttacaaatattttctgcttcgattaaaaaaaaaagaaattaaaagaaaaaaaggcaaagtagtagttgaatgagtgctgatcttgattactcttggtttgctggagaacgatggcaccagagctcgtaagggctgcgatctctataataaagatattgatttgactttgtaggtgcaagcttacttgtacgatggctactgggaagatattggtaccattgaagccttttacaacgcaaatctggggatcacaaagaagccagtaccagattttaggtaacatttgattaagttattaatgaattgttaagttatcatgtgtttatttgatacacgaaacacccaattgcagcttctatgatcgttcgtctccaatctacacccaacctcgctacttgccgccatcgaaaatgctcgatgctgatatcactgacagtgttattggtgagggctgtgtcataaaggtaacttctttacgaagctctttgctatgctaagatggtgttattacctcgtcttaactgcattattccttcccctttccatagttggaataaccccttttttattttctcctcagaactgcaagattcatcattctgtcgttggcctccgatcttgcatatccgaaggtgcaatcatagaagacacgttattgatgggtgcagattattatgaggtctgtattcaataataaccttgtttatcatgagtttcagatagcttttacctctctttaaagtacttttagatacttaaacatcatgaaaatgttgaaaaactacctttaatggttaatgctaccatttgaaactcatgtaaaactattatcttaaaatatagagccacactctaataaaattacgagaatttatgttcaaagtgggcaatattaatataatttctaactgttttgcactttgttgaggagataaattgaggggtggttagagcataaactgatttgtagttttccttggtggtgctcctcagacggacgccgaccgtaggttattggcggcaaagggtagtgtaccaattggcattggcagaaattcccacatcaagagagctatcattgacaaaaatgctcgcatcggggaaaatgtcaaggtatgtcatgtcaacttttgtttgcttttatttcgtttttaacatttatgttattatatcttatcacgcattttcggggtggggtcgatttccattatctttttactctcaatcaaatccactccttccatagagaaggaacaaatacaagtagacaagaaatgagttcataagagccttttctctctcttaaagttgttttgacggtgggaataaatctatttcaaatcagcttcacatggccgcacacgtttggctttttttttttttttttttttttttttttttttttttttttttttttttnnnnnnnnnnnnnnnnnnnnnnnnnnnnnnnnnnnnnnnnnnnnnnnnnnnnnnnnnnnnnnnntttttttttttttttttttttttttttttttataatctatatattaaaaattatggtataaacttttaattaggtgtctaacaagtccttatctttacgttggtagttctgttttttttttttttttctcataacacttgcctgaaattgtagtttaaaaaaagccaacctgaaatcaactaaagtttgataatttagatttagaaatgataactagtcaaacacaccatttttaaatttcaaagatcaatagaatttgttgagaacttttagaagtgtggagtctaaatacgcatttgagttgagtgtcaatgagttgaatgatttgtgatataattgaatcaaacatttggttgaatttcgaatagttgggtatgaatgaattatgatataattgcctcgacgattgggttgaatgtcgatgagttgggtatgaatgaattatgatataattgactcgacatcttattgattaagtatgtcgatgagttgggtatgaatgaattgctatataattttctaactgggcgattgatattcatcgcagatcgtgaacggagacggtgtgcaagaagcagcaagggagacagacgggtacttcataaagagtgggatcgtgacagtaatcaaggatgctttgattcccagtggcactgttatctaa

SEQ ID NO: 65 - DNA, 1575 bp, Cucurbita maxima
  source 1..1575: mol_type = genomic DNA
atggcgtcttctattgctgcaggtggagttctcagagtttcgacgacgtcggggtcttcgtcgtcgaacggcggttctaaccgtgtaaggggaagcagcgttcgaagcctctcgttcggtgcgtctctcatttctggtgacaagattgattttagagcttctgctttgggatcacgacgtttttctggtggtagagcggcacctttgatcgtctctccgaaagcggtttctgattccaagaactctcagacttgcctcgatcccgatgctagccggagtgtgcttgggattattctgggaggtggtgctggaacgcggctctatccgctcactaagaagcgggcgaagcctgctgttcctcttggagcaaactacaggctgattgacattcctgttagcaattgtctcaacagcaacatttccaagatttatgtcctaacgcaattcaattctgcgtctcttaatcgtcatctttcccgagcttatgcgagtaacatgggcggttacaagaatgaaggattcgttgaggttcttgcggctcagcagagtccggagaatccaaattggttccagggaacagctgatgcggttaggcagtatttatggctatttgaagaacacaatgttttggagtatctagttcttgctggcgatcatttgtaccgaatggactacgagagatttattcaagcacacagggagactgatgcagatatcactgtagctgcacttcccatggatgaaagccgtgctactgcatttggtctgatgaaaattgatgaagagggacggataattgagtttgcagagaaaccgaaaggagaccaattgaaagcaatgcaggttgatactactattttgggtcttgatgatgagagagcgaaagagatgcctttcatagccagcatgggtatttacgttatcagtaaggatgtgatgctgaatcttctaagagaaaaatttcccggagcaaatgattttgggagtgaagttattcctggtgcaacttccattggaatgagggtgcaagcttacttgtacgatggctactgggaagatattggtaccattgaagccttttacaacgcaaatctggggatcacaaagaagccagtaccagattttagcttctatgatcgttcgtctccaatctacacccaacctcgctacttgccgccatcgaaaatgctcgatgctgatatcactgacagtgttattggtgagggctgtgtcataaagaactgcaagattcatcattctgtcgttggcctccgatcttgcatatccgaaggtgcaatcatagaagacacgttattgatgggtgcagattattatgagacggacgccgaccgtaggttattggcggcaaagggtagtgtaccaattggcattggcagaaattcccacatcaagagagctatcattgacaaaaatgctcgcatcggggaaaatgtcaagatcgtgaacggagacggtgtgcaagaagcagcaagggagacagacgggtacttcataaagagtgggatcgtgacagtaatcaaggatgctttgattcccagtggcactgttatctaa

SEQ ID NO: 66 - protein, 524 aa, Cucurbita maxima
MASSIAAGGVLRVSTTSGSSSSNGGSNRVRGSSVRSLSFGASLISGDKIDFRASALGSRRFSGGRAAPLIVSPKAVSDSKNSQTCLDPDASRSVLGIILGGGAGTRLYPLTKKRAKPAVPLGANYRLIDIPVSNCLNSNISKIYVLTQFNSASLNRHLSRAYASNMGGYKNEGFVEVLAAQQSPENPNWFQGTADAVRQYLWLFEEHNVLEYLVLAGDHLYRMDYERFIQAHRETDADITVAALPMDESRATAFGLMKIDEEGRIIEFAEKPKGDQLKAMQVDTTILGLDDERAKEMPFIASMGIYVISKDVMLNLLREKFPGANDFGSEVIPGATSIGMRVQAYLYDGYWEDIGTIEAFYNANLGITKKPVPDFSFYDRSSPIYTQPRYLPPSKMLDADITDSVIGEGCVIKNCKIHHSVVGLRSCISEGAIIEDTLLMGADYYETDADRRLLAAKGSVPIGIGRNSHIKRAIIDKNARIGENVKIVNGDGVQEAARETDGYFIKSGIVTVIKDALIPSGTVI

SEQ ID NO: 67 - DNA, 4138 bp, Cucurbita maxima
  source 1..4138: mol_type = genomic DNA
tattacccttccacaaccacgttctatgctaccaaaagcgtggtaaatgacgtaataaagtgaaagttggaaggttagaatcgaccgtaaaaccacaccatatatcagtggcgaagcaaatctgtcgtttggtagtagagttgcagagctgatctctgcttttcattttccggccaccggctcaccggagaagataccgggttttgctttctgttttttttccgatttgaattgactgctttaatggcatcctcaatggctgcagctggagttctcagacttcccatgacggcgtcaagcggcagttctaacagtgcaaggagaagcaaccttcggagtctttcgttctctgaatctctcatttctggtgacaagatcgactttagagcttctggtttgggatcacgacgtgtttccggtggtagagggccttctttgatcgtctctcccaaagcggtttctgattccaagaactctcagacttgcctcgatccagacgctagccgggttggtattcttgtttctttgttccattcgtgttattgattggaatccgagagtttcgttgcttattgttcttgatgcgtttccggacttaacttcgtttgattttctgttttgagccgccgcgaattgattttcgattatatgttttgtgttgtttagagtgtgctagggattattcttggaggtggtgctggaacgcggctgtatccgctcacgaagaagcgagcgaagcctgctgttcctcttggagcgaactacaggctgatcgacattcctgttagcaattgtctaaacagcaacatttccaagatttatgttcttacgcaattcaactctgcttctcttaaccgacatctttctcgagcttatgcgagtaacatgggcggttacaagaatgaaggatttgttgaggttctagccgctcagcaaagtccggagaatccaaattggttccaggtgatgaaatcaagcttagatttatgctttcatgatcaatacgaatccaaattggttctctgaacgctagcattaaaacttccgaatccattttcttcaagtttctcgaaaagtgatgaaatcaagcttaaatttatgctttcatgatcaatacgaaaattggaaaaactgaaacctatcattttgttctgaaattagaacttccgaatacattttctgaaacctatcatctgatagtttgacaccataatctgtagggcacggctgatgcagttaggcaatacttgtggctatttgaagagcataatgtattggaatatctagttcttgctggtgatcatttgtaccgaatggattacgagagattcattcaagcacacagggagactgatgcagatatcactgtagctgcattacccatggatgaagagcgtgctactgcatttggtctgatgaagattgatgaagaagggcgtatagttgaatttgcagagaagccaaaaggagagcaattgaaagcgatgaaggtgaatccaaattttctatcctctcacgtttggctggtaattactctcggcgaccaatctttatcattgctatgtcttctttcttacaggttgatactactattttggggcttgatgatgagagagcgaaagagatgccttacatagccagcatgggtatttacgttatcagcaaggatgcgatgctgagtcttcttagagataaatttcccggagcaaatgattttgggagtgaagttattcctggtgcaacttccattggaatgagggtaagcatataattctcaagacaagtcataatatatatcatccatatattttgtaaatttcttggtgggaatgtggtggcagttataaagggtgttagagctaggcaccgggtggtgtaccagagcaaggacattgaaacctacccatagtgggcacattttaaaaaccttgggggaggtctgaatgggaaagcccgaaaaagacaatatctactagcggtgggcttgagttagggtggattgtgagatcccacatcggttgggaagggtgtggaaacctctccccaagtgggcacgttttaaaaaccttgagaggagaattccgaaagggaaagcccaaagaggataatatctgccatcggtaaacttggtctattaaaagtatcaaatgatggaattgtccggtgcagagtctaaacatttcacgtatgatcaatactaggattcagaagagagaaaaggaaaagaaaatagacaaagtttagactaatgagaattctgatcatggactcctgtcactttgactgttcttggtttgctacagaaaaatgactgtaaagctacttttattgggccatgatctctataacaaagatgttgctttgacatgacaggtgcaagcttacttgtatgatggctactgggaagatatcggtaccatagaagccttttacaatgcgaatctggggatcacgaagaagccaataccagactttaggtaatttagtcaagtttgattcagtttccctattttgttaccaataagctgttaacttatcatttgttatttgatacacgaaacacccaactgcagcttctatgatcgttcatctccaatctacacgcaacctcgatatttaccgccatccaaaatgcttgatgctgatatcacagacagtgttattggcgagggctgtgtcattaaggtaactcccttacaagcaaagtagttgtttgctatgctaatggcgttgttacattgtcttcactgaaatcaatcttcttttattctgtcattagagctgcaagattcaccattctgtcgttggccttcgatcttgcatatcagaaggtgcaatcatagaagacactttattgatgggtgcagattattatgaggtgtgtgaccatataataaccttcattgattggagcctgtctgttttgcacttagtttgttggcagtaagaactgagaagttgtgagcttttactgatttgttttactgtggtgttcctcctcagacggatgccgaccgtagattattggcggcgaagggaagtgtaccaataggcattggcaaaaactctcacatcaagagagctatcattgacaaaaatgctcgcattggggaaaatgtcaaggtttggcacgtcaacttgctttgtttttattcatttattttgtgatgctcatcttcaagttcttattatatcatatcagtcattcttggggtggggtctcatcgggatcttgatttccattatctttttgactatcaatcaaaccctctgatttcattgggaaagaacataaagaacacagacaagtagacaacaaattgagttcatcaatgcttcctttttctcctaaagtagttttgatggtgggaacaattctacttcacatggctgccatattcatttcttatttcaaaattaggaagctataaaaaaaacaacgttcatttatgaatgatatatttaaccttgtaaacttttttatatttaattgtgtttaataggtggttgttctattgaacacttgataatggaacaacttgaatcctaccaaatttaacaactataatctgcactctcttattgaacacacacacacacacacacaacaaatccttaaattacaatgatcaggaagtacttacatttcttgatataagtccaagtctaccgctagtcgatattgtcttttttaggtttttccttctttcgcagttttaaaatgcccctgctagagagggtgttcacacctttataagaaatgcttcgttctcttctctaactaacgtgagatctcaaaaagaaaaaaagaaaaagaaaatatatataacacacgagggtgggaataacagtagtacgagttcgttataacagtagtatgagttcgattgtgagtaataataataagcatatgaatgttgttgggtgtgtgatattgaatgaagtttgtgatctcagattgtgaatggagacggtgtgcaagaagcagcaagggagacagatgggtacttcataaagagtgggatcgtgacagtaatcaaggatgctttgattcccagcggcactgttatctaaggccacagcagaacagagcagacatgattttcactctctctctctctctctctctctctctctc

SEQ ID NO: 68 - DNA, 1563 bp, Cucurbita maxima
  source 1..1563: mol_type = genomic DNA
atggcatcctcaatggctgcagctggagttctcagacttcccatgacggcgtcaagcggcagttctaacagtgcaaggagaagcaaccttcggagtctttcgttctctgaatctctcatttctggtgacaagatcgactttagagcttctggtttgggatcacgacgtgtttccggtggtagagggccttctttgatcgtctctcccaaagcggtttctgattccaagaactctcagacttgcctcgatccagacgctagccggagtgtgctagggattattcttggaggtggtgctggaacgcggctgtatccgctcacgaagaagcgagcgaagcctgctgttcctcttggagcgaactacaggctgatcgacattcctgttagcaattgtctaaacagcaacatttccaagatttatgttcttacgcaattcaactctgcttctcttaaccgacatctttctcgagcttatgcgagtaacatgggcggttacaagaatgaaggatttgttgaggttctagccgctcagcaaagtccggagaatccaaattggttccagggcacggctgatgcagttaggcaatacttgtggctatttgaagagcataatgtattggaatatctagttcttgctggtgatcatttgtaccgaatggattacgagagattcattcaagcacacagggagactgatgcagatatcactgtagctgcattacccatggatgaagagcgtgctactgcatttggtctgatgaagattgatgaagaagggcgtatagttgaatttgcagagaagccaaaaggagagcaattgaaagcgatgaaggttgatactactattttggggcttgatgatgagagagcgaaagagatgccttacatagccagcatgggtatttacgttatcagcaaggatgcgatgctgagtcttcttagagataaatttcccggagcaaatgattttgggagtgaagttattcctggtgcaacttccattggaatgagggtgcaagcttacttgtatgatggctactgggaagatatcggtaccatagaagccttttacaatgcgaatctggggatcacgaagaagccaataccagactttagcttctatgatcgttcatctccaatctacacgcaacctcgatatttaccgccatccaaaatgcttgatgctgatatcacagacagtgttattggcgagggctgtgtcattaagagctgcaagattcaccattctgtcgttggccttcgatcttgcatatcagaaggtgcaatcatagaagacactttattgatgggtgcagattattatgagacggatgccgaccgtagattattggcggcgaagggaagtgtaccaataggcattggcaaaaactctcacatcaagagagctatcattgacaaaaatgctcgcattggggaaaatgtcaagattgtgaatggagacggtgtgcaagaagcagcaagggagacagatgggtacttcataaagagtgggatcgtgacagtaatcaaggatgctttgattcccagcggcactgttatctaa

SEQ ID NO: 69 - protein, 520 aa, Cucurbita maxima
MASSMAAAGVLRLPMTASSGSSNSARRSNLRSLSFSESLISGDKIDFRASGLGSRRVSGGRGPSLIVSPKAVSDSKNSQTCLDPDASRSVLGIILGGGAGTRLYPLTKKRAKPAVPLGANYRLIDIPVSNCLNSNISKIYVLTQFNSASLNRHLSRAYASNMGGYKNEGFVEVLAAQQSPENPNWFQGTADAVRQYLWLFEEHNVLEYLVLAGDHLYRMDYERFIQAHRETDADITVAALPMDEERATAFGLMKIDEEGRIVEFAEKPKGEQLKAMKVDTTILGLDDERAKEMPYIASMGIYVISKDAMLSLLRDKFPGANDFGSEVIPGATSIGMRVQAYLYDGYWEDIGTIEAFYNANLGITKKPIPDFSFYDRSSPIYTQPRYLPPSKMLDADITDSVIGEGCVIKSCKIHHSVVGLRSCISEGAIIEDTLLMGADYYETDADRRLLAAKGSVPIGIGKNSHIKRAIIDKNARIGENVKIVNGDGVQEAARETDGYFIKSGIVTVIKDALIPSGTVI

SEQ ID NO: 70 - DNA, 4287 bp, Cucurbita maxima
  source 1..4287: mol_type = genomic DNA
atggcggtctctaccgactgtcggattgccttatcggccgccgcgcatgtgcggaatgcacgtgcgactgcggtggtggtcgggaggaagttgaggtctgtggagtttgccaatggcgagttcatgggaatgaagctcaaggttaaggagtcgaaaggagtagccgctgctgctaagagtgttgggcgacgtgtttgtatgtctctcaccactaatgtagccattggcgacgctgcggtaattttgagaactctgctgagcagtattttaatcttctttatacgatatggaacattagagtttgaacttgccagatttgttttgtttttattgcgttgtgaactttgttgtttcggtaattttgcggagatttgcaactacaccttcgttgcttcgcattgatttagtttgtttgtccacattttgaaatgctacggcttcgtgttggtgatggtttgtctctgtatttcgataaattttcctagggcgttcgtctccaagatatggtctgaactttagagaagactgctaattaggatgctaatgtctagccttctaccttttgaattagagaccagtgcgatttatgtggttaccatctactatgaccttagatcattcgattctttgttcaggacttcacgccttctgattccagttgaatctaacaacgtgctgcaatttggacttagcctgacctgtgtatatatttatatatatttgaacaaactcaaagaataccattgtgaagtgaatcaagggacattgtgaagtgaatcaagggactgaataattttagactttcacaatcttttgtttcccccattcctaagtgagaccccttaaaagtattgattaattgggaggagtgatatcagaaactcaccctgctttctatctcttactagcagggaaatattgtgtataattcactctcaaaatgatctgggacaacagttaatgtgttgctgcttgtgaattttgtttagttgagagaaatagatacggaaaaaagggaccctcggactgtggttgcaatcattctgggaggaggagctggaactcgtctatttcccctcaccaagcggcgtgccaagcctgctgtaagttttccctgctaaactcaatttttcttactgttatttatcattgaagtattgtaactgctagacactgcgaaatcaagtgggttccagttatttttccctcttcaggttcatttagtttagatgattggaaatagtgacattttattttcatatattcatatgggattttgttacattggctgctctgtgctttcttatgaatttccattacaggttccaatcgcaggagcatataggctgatcgacgtgccaatgagcaactgtatcaacagcggaatcaacaaagtctacatcctcactcagttcaattcagcatcactgaataggcatcttgctcgagcttacaactacagtggtggagtcacctttggagatggctatattgaggtacgtttccttcacttaaaacatgtcccacaatcatattcccaaagaaaaatatgactaaaggattcagcttcatatcaggttcttgcagccacccaaacgccaggggtggctggtaaaaattggtttcaaggtacagcagatgccgtacgacaattccactggctctttgaggtacaaaaaaaaccttatctaataacataatgatgtaagatatgatattgagatgcatataaagaaatgattgtttgtgacattttggttgttccgtatacagtaagtgatgaaagaaaaaattgggttaaatacgtattaactttccttggggtcatatctgacatagaaaacaggccttaatctaaggactgagatagtaataagccttgcctgctgcaggacgccagaagcaaaaatattgaagatgtgttgattctgtctggagatcacttgtacagaatggactatatggattttgttcaggtaaggctttaaatgcacttaaaaaaccaatcttcatcctgttgttacattcagtttttatgtatcttctttacattggcagaatcataggcaaagtggtgcagatatcactctttcttgtcttccaatggacgataggtgagtgacaacttaatgtttttctttcggacagttagctaaaaaatcattgatcctgaaggccccacagaccttgatcatgcaatcagttcaaacctttttctgtttgcaaaaacttgagtatctaggacttaaggttcaaagaattacagaaatgtgtaacgaaaggagaagagcatcttttcttcacatttactttatttcaaacaatcacattactttgtattatagccgagcctcagactttggtctaatgaagatagacgacaaaggaaggatactttcattcagtgaaaagcctaaaggagaggacctgaaggccatggtaacattcaaaatttccttcactaaaaatttcaaatgggtggaggaacttaaaatgattgttctgatgaacctaaactatatcatttccattcatattgaaggcagtagatacaaccattttgggactctcgaaagaagaggctcagaagaagccatatattgcttccatgggagtgtacgtctttaagaaggagatacttttaaatcttttaaggtattgtgcatctaatgctatttatgaatctagtaattttatacaaactcaaaggattttgttcaacacgaagtattagactttgcttgaatttgttcaagtcagatggcgctttccaactgctaatgactttggatctgagataatccctgcctcagccaaagaatactttataaaggcttatctcttcaatgattactgggaagacattgggacaattagatccttctttgaagcgaaccttgccctgactgagcaagtaaagcaactaaacactttccattttaaattttctactctctctctctctctcatccatcaggacttttcacaagttattcgaattgtagtttgacccttttttatcgttattcttgcttgtttgcttatacctttcagccaccaaggtttagcttttatgatgctgcaaaaccgatgtttacttcaagaagaaacttgccaccctcaaagattgaccagagcaaggtcggtagtattctgcaatcgtctaaatctctattataaaatcataaaactctaataaaaacctccattcttttttaacgaaatatgctgtatttggccctaaaacaacactgcatttggcaattggagaaaagaaaccagttattaacaacggctgagcatttttctggttacttgacaatttttctttgctctttatgttgagataatatttgggtcctttgcagatcgtagattctatcatctcacatggaagcttcttaagcagttgctctatagagcacagtgtcatcggcattcggtccagaattaactctaacgtgcacttgaaagtacgcatctcatggaacttttgcaagtgataatcaaactaaggagttgaattcagtcctttgcaaaacataagttttttctgaacggagtgatttttgatgagatgcaaaatgcaggacactgtgatgcttggagcagattactatgaaactgaagctgaagttgcatctttgttagctgaaggaagagtaccaataggaataggagaaaattcaaaaataaagtacgaacaccacaaaaccaaaagtattttggagcctgcttttctcattcctcattgttgatataaatattatgtgcagggactgcattattgacaagaacgctagaattggaaagaacgtggtcattgccaattcagaggtaccgttaccatagagtaaaaaagggatttgttgcattcgtaaaaccaatgagatttcaattgacctcgaaatgaatgaattatgtattactggaaacatattgcagggcgtacaagaggcggatagatcttcagaaggcttctacatacgttctggcattaccatcatctcaaaaaatgcagtaattgaagatggtttggttatataataaacatgtttatccctcgttgatgaacatgtcgatccttcattgatttgtattatttctttcgttaatgtttaataagaatgcagaggagatgtaacaacaaaatatatctatggatgtgattcttcttcagcacaacgaccatgccaagctgagggcatataacatatcgaggtaaatgatccctcctctttgctctgtctctaagattatgatcattcttttctaaagcctggtgtg

SEQ ID NO: 71 - DNA, 1581 bp, Cucurbita maxima
  source 1..1581: mol_type = genomic DNA
atggcggtctctaccgactgtcggattgccttatcggccgccgcgcatgtgcggaatgcacgtgcgactgcggtggtggtcgggaggaagttgaggtctgtggagtttgccaatggcgagttcatgggaatgaagctcaaggttaaggagtcgaaaggagtagccgctgctgctaagagtgttgggcgacgtgtttgtatgtctctcaccactaatgtagccattggcgacgctgcgttgagagaaatagatacggaaaaaagggaccctcggactgtggttgcaatcattctgggaggaggagctggaactcgtctatttcccctcaccaagcggcgtgccaagcctgctgttccaatcgcaggagcatataggctgatcgacgtgccaatgagcaactgtatcaacagcggaatcaacaaagtctacatcctcactcagttcaattcagcatcactgaataggcatcttgctcgagcttacaactacagtggtggagtcacctttggagatggctatattgaggttcttgcagccacccaaacgccaggggtggctggtaaaaattggtttcaaggtacagcagatgccgtacgacaattccactggctctttgaggacgccagaagcaaaaatattgaagatgtgttgattctgtctggagatcacttgtacagaatggactatatggattttgttcagaatcataggcaaagtggtgcagatatcactctttcttgtcttccaatggacgatagccgagcctcagactttggtctaatgaagatagacgacaaaggaaggatactttcattcagtgaaaagcctaaaggagaggacctgaaggccatggcagtagatacaaccattttgggactctcgaaagaagaggctcagaagaagccatatattgcttccatgggagtgtacgtctttaagaaggagatacttttaaatcttttaagatggcgctttccaactgctaatgactttggatctgagataatccctgcctcagccaaagaatactttataaaggcttatctcttcaatgattactgggaagacattgggacaattagatccttctttgaagcgaaccttgccctgactgagcaaccaccaaggtttagcttttatgatgctgcaaaaccgatgtttacttcaagaagaaacttgccaccctcaaagattgaccagagcaagatcgtagattctatcatctcacatggaagcttcttaagcagttgctctatagagcacagtgtcatcggcattcggtccagaattaactctaacgtgcacttgaaagacactgtgatgcttggagcagattactatgaaactgaagctgaagttgcatctttgttagctgaaggaagagtaccaataggaataggagaaaattcaaaaataaaggactgcattattgacaagaacgctagaattggaaagaacgtggtcattgccaattcagagggcgtacaagaggcggatagatcttcagaaggcttctacatacgttctggcattaccatcatctcaaaaaatgcagtaattgaagatggtttggttatataa

SEQ ID NO: 72 - protein, 526 aa, Cucurbita maxima
MAVSTDCRIALSAAAHVRNARATAVVVGRKLRSVEFANGEFMGMKLKVKESKGVAAAAKSVGRRVCMSLTTNVAIGDAALREIDTEKRDPRTVVAIILGGGAGTRLFPLTKRRAKPAVPIAGAYRLIDVPMSNCINSGINKVYILTQFNSASLNRHLARAYNYSGGVTFGDGYIEVLAATQTPGVAGKNWFQGTADAVRQFHWLFEDARSKNIEDVLILSGDHLYRMDYMDFVQNHRQSGADITLSCLPMDDSRASDFGLMKIDDKGRILSFSEKPKGEDLKAMAVDTTILGLSKEEAQKKPYIASMGVYVFKKEILLNLLRWRFPTANDFGSEIIPASAKEYFIKAYLFNDYWEDIGTIRSFFEANLALTEQPPRFSFYDAAKPMFTSRRNLPPSKIDQSKIVDSIISHGSFLSSCSIEHSVIGIRSRINSNVHLKDTVMLGADYYETEAEVASLLAEGRVPIGIGENSKIKDCIIDKNARIGKNVVIANSEGVQEADRSSEGFYIRSGITIISKNAVIEDGLVI

SEQ ID NO: 73 - DNA, 4520 bp, Cucurbita maxima
  source 1..4520: mol_type = genomic DNA
atggattcgtttggtgtgaccttgaaggccaatacgatgccgtttagagccagcagacagtgtggtaggaaggtgcaatgctccgggttctggggagagagtattgagagaagcggaaagtgcaaacagatacagatgaatgcctactcgcggaagaattcttattcttcttcccatagcagggctagaaagcttacggcgggagttgctcattctgttctcatgtcagaagttagtgaggaaacaacggtaagtggatttagtactttgatattcaaaagtacataaacttttatcggaacttttgactccaaattttacctcagactttacaggcttctatatttgagactcctagagctgatccgaaaaagattgcttcaatcatactgggtggaggtgccggaactcgcttgtttcctcttaccagccagcgcgccaaaccagcggtaaccacctctaccaaattgcttatataagaataatttcacaaacttggagagcgtttttaattggggagatgtatttagatggttgtactagatacttgataatagaagctattgatccaacccaggagaacaaactggtgcatgaatttaagcaaataatttttacttgcaggttccaattggaggttgttatagattgatcgacgtgcccatgagtaattgcattaatagcggcatcgaaaagatattcgtcctaacgcagtttaattcgttttctctaaatcgtcaccttgctcgtatttacaattttggtaatggagtgaattttggcgatggattcgtggaggtatgatgactgtacttcccatttttacaatctgcccctgctttgcaattgttttaattagatgggctgcaagttcttggaacccggaagctttcttatgctcatcctctggattcatgtttgagaaacaggttctagctgccactcaaacttcgggtgaaactggaaaaaaatggttccaaggaactgccgatgctgtcaggcaatttatatggttatttgaggtgggtgcctaaaatcttctgagcatgattggaattttcagtcaaatttttttatgtataatgaacgttttatccattgggtttctgcaggatgccaagaccaaaaatgttgagcacactctaattttgtctggcgatcacctttaccggatggactacatggattttgtgcaggtataacgtgaagctaactagtttgtacttgtatttgtatagttgtgctcatttgttcgtgattcaatgaggatttctgatgacttcgtacttgcagagacatattgacacaaatgcagatattacagtttcgtgcatacccatggatgataggtaggcatctttttttatcgttatttccatggataaagtgtttcttaaaactcagatcttcattaatgctttgttattctgcagccgagcatcggattacggtctcatgaaaatagacgacacaggacgtattttacattttgcagagaaaccaaagggatctgacctggaagcaatggttggtaaaattgatgcaatttgtttggtttgtaattagcaatatctttgtctgatcttgttttctatccattatatattgtttattgcagcaagttgacactacagttctaggactttcagatcaagatgctagaaaaaatccttatattgcatcaatgggagtctacgtattcagaactgatcttctattaaagcttttgacatggacttatccttcatgcaacgacttcggctcagaaattattccatcggctgttaaagactacaaagttcaagtgagatcccttttgagaaattttaatctttcattttttccttgtgcttgaattcagaataggaagactttcgacgataatagaactaagtttcccaatgtatccatattgttgtaggcatatctattcaatgactactgggaggacattggaaccgtgaagtcattttttgatgccaacttagcactaacggagcaggtatgtatcccatcgattgtctcctttcttttcctttgtatgggaccaagaatcatctccagctttcttgcttatttagttatctgagttaaatatttatatagaagataagtaccttaactggcctgtgaatttttcaagcaacgccctccctcggatcagaaacaacacatgatcaagtgtcattaagctgatggctttagtgtctttcatgcagcccccaaagtttgagttctatgatccgaagactccattttatacatcacctagattcttgcccccttctaaagttgaaaaaagcagggtaagacatcaaactcgagtttgctctctgcatcatccgtttgattcttgtatcttaaacgaagttgcttttgcagattgtagatgcaataatatctcatggttgcttcctccgagagtgtagtgttgaacactctattgtcggtgtgcgctctcgattggagtacggtgtggagcttaaggtgagccagggttttcttttgaattttcccctccccttcgatactgtaggaacgtacaagaagaatgcaatggcagaagatggttatagatgctgcaatcttttttttccacagattaaactcttactattattgcaagagattctgccatgccgatatgtagagcatcttagcttgtaaaaagttgatcatatttggatttgttggttttgcattctaggatactatgatgatgggcgcggactactaccagactgaatctgaaattgcatctctgcttgccgaaggtaagatcccaatcggcattggagagaacaccaaaatcaggtaatattcagctaccaatacagagcaaaagctatctgcaatctcttattagttaaatataatcgtcactttatcatccgactgtttaccataaacattatttgttcatgggtttggtctgtttctgattttcatttatcttttccgcaggaattgcataattgacaagaatgcgaagatcggaaaaaatgtagttattgctaatactgatgtaagtatgatatttgtgttcttaagggatatttggttcaataagttttgtttcatccatgtttgtatttgtacaaagaaataaccaataaaaggttaggtttggttcttgggtttcataacctgggtggtttcatatgtgaaggtttatataacatgagttcatgcaattcggtagggaggggaaaattaacactttctaagaatttgcaaactctatctaaaccacataaacccatgatgttattttggtcatagtgcctaacaaatgtgtggttgttaacttttcagggcgttcaagaagcagatagaccagaagaagggttttatataaggtccggtatcacagtcacgttgaagaatgcaacaataaaagacggaaccattatttaaatccaaagagaaagctgagagttgaaaggaagcaactgctcgaggctttcaaccgacaatgctcgacaacgagtaaaaggctagcttcttttcttatatgtaaaaagttgaaaggcaacacattcctgagtaagagaacaactttttatttcaatccatatattattagagactacaatattcaaagcgatttagcattagtttttggatgagcaatcctttctctgtgagggagttgtttattcctagccccatttttgttcaatttccaggactatggctgcctccctttaaggcctccccctgtgtcttatgtttctgaggttggcatgcttcttccttattttgatcatctttgcagactcctgagatatcaaattagcagtggaggggtcctcactaaataccacttcatacccatccccataagaacccatcccttcaagtagaagttgccagaacagtgcacccgtagctgctccgccgcccttagcggatgaatacacagctgagtaaacagcatggtaaagtcgatcccttttatcagcgccaccgtcctttttagacattccaaactctgcaaagagaactggcttgtggagaacgttctgtgcatcttgggtgtgttcattgagccaagtgttcaggaatgagagctgatcctcaaaacttgaaccagataaccttcatcaaatcaccaccacgttcatttcagctaataatggattgaagatctcagaataggagccaatgggagaagggttttgttcttaccattcgtcaggatacgagtggactgtggaaaagtcgatttcgggtatctgattgtttgcaatgaaatcggttcccacttggaagttaggattcccttgatgccttgcttggccatagaatccttccaaaccaacttctaataggtgtttcccatcaattgatttcaagtatgcggccatttctttaatccatgcctgcccatcatttcatatatacaactttttaaattgggctatgtcagcttagtaggtataataaatgtataatcaggtgcaactttttgcattaacataattttacttgtaatgagacttcgtctttggctcaaa

SEQ ID NO: 74 - DNA, 1602 bp, Cucurbita maxima
  source 1..1602: mol_type = genomic DNA
atggattcgtttggtgtgaccttgaaggccaatacgatgccgtttagagccagcagacagtgtggtaggaaggtgcaatgctccgggttctggggagagagtattgagagaagcggaaagtgcaaacagatacagatgaatgcctactcgcggaagaattcttattcttcttcccatagcagggctagaaagcttacggcgggagttgctcattctgttctcatgtcagaagttagtgaggaaacaacgactttacaggcttctatatttgagactcctagagctgatccgaaaaagattgcttcaatcatactgggtggaggtgccggaactcgcttgtttcctcttaccagccagcgcgccaaaccagcggttccaattggaggttgttatagattgatcgacgtgcccatgagtaattgcattaatagcggcatcgaaaagatattcgtcctaacgcagtttaattcgttttctctaaatcgtcaccttgctcgtatttacaattttggtaatggagtgaattttggcgatggattcgtggaggttctagctgccactcaaacttcgggtgaaactggaaaaaaatggttccaaggaactgccgatgctgtcaggcaatttatatggttatttgaggatgccaagaccaaaaatgttgagcacactctaattttgtctggcgatcacctttaccggatggactacatggattttgtgcagagacatattgacacaaatgcagatattacagtttcgtgcatacccatggatgatagccgagcatcggattacggtctcatgaaaatagacgacacaggacgtattttacattttgcagagaaaccaaagggatctgacctggaagcaatgcaagttgacactacagttctaggactttcagatcaagatgctagaaaaaatccttatattgcatcaatgggagtctacgtattcagaactgatcttctattaaagcttttgacatggacttatccttcatgcaacgacttcggctcagaaattattccatcggctgttaaagactacaaagttcaagcatatctattcaatgactactgggaggacattggaaccgtgaagtcattttttgatgccaacttagcactaacggagcagcccccaaagtttgagttctatgatccgaagactccattttatacatcacctagattcttgcccccttctaaagttgaaaaaagcaggattgtagatgcaataatatctcatggttgcttcctccgagagtgtagtgttgaacactctattgtcggtgtgcgctctcgattggagtacggtgtggagcttaaggatactatgatgatgggcgcggactactaccagactgaatctgaaattgcatctctgcttgccgaaggtaagatcccaatcggcattggagagaacaccaaaatcaggaattgcataattgacaagaatgcgaagatcggaaaaaatgtagttattgctaatactgatggcgttcaagaagcagatagaccagaagaagggttttatataaggtccggtatcacagtcacgttgaagaatgcaacaataaaagacggaaccattatttaa

SEQ ID NO: 75 - protein, 533 aa, Cucurbita maxima
MDSFGVTLKANTMPFRASRQCGRKVQCSGFWGESIERSGKCKQIQMNAYSRKNSYSSSHSRARKLTAGVAHSVLMSEVSEETTTLQASIFETPRADPKKIASIILGGGAGTRLFPLTSQRAKPAVPIGGCYRLIDVPMSNCINSGIEKIFVLTQFNSFSLNRHLARIYNFGNGVNFGDGFVEVLAATQTSGETGKKWFQGTADAVRQFIWLFEDAKTKNVEHTLILSGDHLYRMDYMDFVQRHIDTNADITVSCIPMDDSRASDYGLMKIDDTGRILHFAEKPKGSDLEAMQVDTTVLGLSDQDARKNPYIASMGVYVFRTDLLLKLLTWTYPSCNDFGSEIIPSAVKDYKVQAYLFNDYWEDIGTVKSFFDANLALTEQPPKFEFYDPKTPFYTSPRFLPPSKVEKSRIVDAIISHGCFLRECSVEHSIVGVRSRLEYGVELKDTMMMGADYYQTESEIASLLAEGKIPIGIGENTKIRNCIIDKNAKIGKNVVIANTDGVQEADRPEEGFYIRSGITVTLKNATIKDGTII

SEQ ID NO: 76 - DNA, 5930 bp, Cucurbita maxima
  source 1..5930: mol_type = genomic DNA
aatgttcttaagtattcctggttgcatgcggcgtagaaatcgcggatggtagttacaaatcgggtgttgtgggaaatgcaaagaaggaagaagaagaagccgttgatccatcgagttcattttcctttttttagtataaaagcctcctgattcagagacctcttctcactcacttacatgcccttattccaccgctttcttcgtaatctcccacgcttttcccttcttcttgccccatttcattcaatccctctgccttctcctcctccgattgcttcccctttcaatttctcaggtgatttcttgcatttttcttttgtttctgtgatctgtacgctaaatgcttgctttgatttggggttttgctttattgggatttgaatttgattgtattttgtgccatttctgagcgattgatttgtatttgtatttggatttggtttttgtgtagtgagctatggattcctgctttttgtccttcaaacccaattcccatctgtgtctgaatcagaatggattttatggggagaaagttagaggaagtttggataataatgctttgtttaatcgcttagcaaagagtttgaaatctgagaataaagctttgaagctcacctctaatggtgctgcttctgttgttgctacactaaatattaccaaggaggctgcggtgagtttattgttatttctggaagtttgatcaatgcccatcttgattttgattgattcttaactgtttggattggatttgaagagcattcaagctccaccgtttcatacagtcaaagttaaccccaagaatgttgcttcgatcatattgggaggaggtgctgggactcatctgtttccccttactagaagattggccacacctgctgtaagttcttactaatgttgctgtgtttcttagaacagatgaagttattttttctatcaaaattatcgtgagattccacctcgattggagagaggaatgagtgtctgtgaagctccaaagggggtggattgtgagatcccacggttagagagggaacgagggccagtgaggacgctagcccccaaggggggtggattgtgagatcccatattagttggagaggggaacgagtgctagtaaggacaacgggcccccaaagggatggattgtgagatcccacattggttggagtggagaacgaagcattccttattgaggtgtggagacctctccttgattgacacgttttaaaatcatgaggctgacggtgatacaagccaaagcggataatatttactagtgtaggcttgggctgttacaagtgcgttgaaaatgcggttttcaatttaacttctgccattttacctggtgtaatcatgtagttcgaattattcatcatcaagtactatctcgtttgtttagtagactaatcggttggttgagttatgcaatttgatcgtctactcgtttttgcaggttccagttggaggatgctataggcttatagatattccaatgagcaactgcctcaacagtgggatcaacaaaatttttgtgcttacccagttcaactctgcttcgttgaatcgtcatatttcacgaacgtacttcggaaatggtgtcaactttggagaaggattcgtggaggtatactgataaactgtgaactttaatgataaaaagagggagctaatcttttcctagcatgtcgaaaatcgttatgatcctgagcttactctcgatatacaggttcttgcagccactcaaacacccgggaaatctgggatgtactggttccaaggaacggcagatgctgtgagacaatttatttgggtgttcgaggtacataacagagttattttcttttgttatggctgaacttctctttatcgatatcgtaaagctattcgcgtattaattcttgtaggatgcgaagaacagaaatgtcgaaaacattctaattttggcaggggatcacatgtacagaatggactatatggactttgtgcaggtaacaactttatttttgagcgcccgtgctcattagtgataatagcttctgttctatatgtcgaaaatcatatcatatgctacgattggacacagaatcacgtggatagaaacgctgatatttcaatctcgtgcgcgcctgtggacgacaggtcttatcaacataccttttaccttttgattccgttttttacttagaattttagagctctcatacgttatttcatttactccgcaactttgcagccgtgcatcggactatggattggtgaaactcgatagtagaggtcgaattatccagttttcggagaagccaaagggtgcccatttgaatgcaatggtaaaatattgaagaacatatagaatggttataaactatgaatcttgccatacatggaaatgatctttcttggctgaataaggctcgtgcgtttaatcaccctaactgatcggttttcttcagcgagtggatacaacttcattaggtttgtctcgggaggaggcgttgaagtccccttacattgcatcaatgggcgtttacgttttcaaaacggacgttttgctaaaccttttaaagtggagatatcctacatctaacgacttcggctcggaaatcattccagcagctgtaaaggagcacaacgtccaagtaagaatcgagtccgactgctagaccttaaaagaacttttgaaccgaccgagctttttttatttcgacttcattgatatattcatatgagtgtaacttctctttattgcttcctttttctccaaggcatttgtatttagagactattgggaggacattggaacaatcaggaccttctatgaagcaaacttggccctcacggaagaggtagctttcatttactgaatttttcactttagtcgccattgattatcgatttttcacttgtttacagtttcctaagtttgaattttacgaccccaagacgccgttctatacatcgcctcgatttttaccaccaaccaagattgataaatgtcaggtatatcaataaaaccaatctgattctcggctctgtctttaggactctggtcttaagcttcgactgggttttgcagatcgttgatgccataatctcacatggatgctttctgagagaatgtagtatccagcattcggtaatcggtgaacggtcaagattagactacggtgtcgaacttcaggtgaacactagagtttgtaagacacggcggctggttagatgaacacgagagtttgtaactcaggttgctgtcttctttcttacaggatactataatgatgggtgcagacaattaccaaactgaactcgaaattgcagctctgctagctgagggaaaagtccctattgggatcggacaaaacacgaaaatcaggtaagttgtcgactcgaaccttgaaatactgcacaacaacgtttcaacagtgttcatgtcgggcttgggtttgctgcaggaactgcataatcgataagaacgtgaagattgggaaagacgtcatcatcatgaacaaagaagtaagcgcatccaacccggttcgacaaactacattcaatcccaaaacctccaggcattcttaatctgtatctttgtcaatttcagggtgtcgaagaagcagatcgacccgaacacggattctacgttcggtcgggaatcaccattataatggaaaaggcaacgatcaaggacggcacggtcatataaaccggtctcgaggtctcagtctcgacatgtttagaggggaagaacatgctctgtgacagctgaagaatggcatttgcagcatttaggctgagaagaagaaggattatagtgacaaagatgaatttatagataggtttggagcaaagaaagttgctttttagtataacatataaggggagatgttaactggagattgcaaatctttagtgtaattctctctaaataagagtggctactactactcacccagattcatctttatgtacataaaaactatatctttatgtattcttcataattgttgccttttatcatttcattgatatttggtgacacttccatattgaaatcaatatcaaaatttttggttaatggcatgttcactcctatctttgctaaattagtgtatgttttgtatcatatcgtgtggtgtggtatgtagtattaggaaaggatgaatcgctcttgaaaaggaatctattgattctctcccaattttgttaagttgtaggtgtgacaatttatgcgacctctggcacgactatggattggtcccgcctaaagtgagtgaggtgaacagtgagttagtgaaagaagggaagctatgcacaaggagaggaagcttagcaattggatatctcaaacccacttgagaaagagagtttcggatatagcaaaagagagacaacattaatgagactgatttcactcttttacaaggtaaataagaaggtaaagacggtttgtggaggtcgaggactgaattaatagacaccaaacacgagggttgtgctcatcgcatgacttttctagttgccgacgatagccaagcactcactatctatgtccacattttcaacgactttctgacaaaaaaaaaaaaaaattattagggtcatgcactcgtttataattacgatcctgtactcgttccaagagaatagttgcatcgtaaattagcttgaatacaaaaacatgcccaacaaaaaagaatgtccgatggaattactcgatgttgaattaaaacaattataaaaataaaatatggcttagcaaagtgaaatgctaatcttttagggggaaggtctaatattgcaaacttgcataatatgtcccattttttattctctttcttttcctttttggagaaatctattttttgttgtcagcaactcaaactccaacctgtcgttttcacagaaaaggattatgacacatggcagacaaagatccatttcagatcaacacgtggcagtccaagatccatttaagatcgtggagaagaatatagccactgcacacacttcctctacccctgcagttaaagatctggagaactagaaaccagaagaagaagaagaagaagaagaagaagaagacgcaactggatcacaaaaaatggctcagcctgaatcgaaccaatcccaaagcaccccggagatcgattccgccacagctttacaacaatatttcgatcgcatccccgtcagttccatctccggcatcaaatccgccctcggtaaccccatctccttcactattttcctttgcacgcaacctgtttgttttaatgccaattcagaaatcccttctctggtatgatctaatctgtttctttccacacaagtcttggaagtgaagactggggactgtgttaaggatgcaataagggttatgtttaagaagaatgtggattgtgctctcatagccgatgtcaccagcgagggatttcccgatcggttcattggctttgtccatctctcaagcttgcttctatggtgtattcaggtagtttgaaatttcaatgggtattgttttgaagggttttgatcttgaatggctgaatgatgaactggcttaggagattgggaagttggaggatgaaatcaaatgcattcaagcagaggaaactcaagagaatgttcgtgggattctgagttttcttgaacagaatccacatattggccaaaccaaggtgtttgtttcttgtttgattgttgcattccatggtttctttctgataagttttggtgggtttttggggaaatttcaggtgggtgagttagggaagacatttttgtgggatccttatttcccagtagggatgagggatacacttttccatgttctgctattgctttcaaagcatcggttgcaggctgttcctgtagttcagctctgcaattctcacgtcgttggtttcattacaaaggtactttctactgcttcataa

SEQ ID NO: 77 - DNA, 2232 bp, Cucurbita maxima
  source 1..2232: mol_type = genomic DNA
atggattcctgctttttgtccttcaaacccaattcccatctgtgtctgaatcagaatggattttatggggagaaagttagaggaagtttggataataatgctttgtttaatcgcttagcaaagagtttgaaatctgagaataaagctttgaagctcacctctaatggtgctgcttctgttgttgctacactaaatattaccaaggaggctgcgagcattcaagctccaccgtttcatacagtcaaagttaaccccaagaatgttgcttcgatcatattgggaggaggtgctgggactcatctgtttccccttactagaagattggccacacctgctgttccagttggaggatgctataggcttatagatattccaatgagcaactgcctcaacagtgggatcaacaaaatttttgtgcttacccagttcaactctgcttcgttgaatcgtcatatttcacgaacgtacttcggaaatggtgtcaactttggagaaggattcgtggaggttcttgcagccactcaaacacccgggaaatctgggatgtactggttccaaggaacggcagatgctgtgagacaatttatttgggtgttcgaggatgcgaagaacagaaatgtcgaaaacattctaattttggcaggggatcacatgtacagaatggactatatggactttgtgcagaatcacgtggatagaaacgctgatatttcaatctcgtgcgcgcctgtggacgacagccgtgcatcggactatggattggtgaaactcgatagtagaggtcgaattatccagttttcggagaagccaaagggtgcccatttgaatgcaatgcgagtggatacaacttcattaggtttgtctcgggaggaggcgttgaagtccccttacattgcatcaatgggcgtttacgttttcaaaacggacgttttgctaaaccttttaaagtggagatatcctacatctaacgacttcggctcggaaatcattccagcagctgtaaaggagcacaacgtccaagcatttgtatttagagactattgggaggacattggaacaatcaggaccttctatgaagcaaacttggccctcacggaagagtttcctaagtttgaattttacgaccccaagacgccgttctatacatcgcctcgatttttaccaccaaccaagattgataaatgtcagatcgttgatgccataatctcacatggatgctttctgagagaatgtagtatccagcattcggtaatcggtgaacggtcaagattagactacggtgtcgaacttcaggatactataatgatgggtgcagacaattaccaaactgaactcgaaattgcagctctgctagctgagggaaaagtccctattgggatcggacaaaacacgaaaatcaggaactgcataatcgataagaacgtgaagattgggaaagacgtcatcatcatgaacaaagaaggtgtcgaagaagcagatcgacccgaacacggattctacgttcggtcgggaatcaccattataatggaaaaggcaacgatcaaggacggcacgaaaccagaagaagaagaagaagaagaagaagaagaagacgcaactggatcacaaaaaatggctcagcctgaatcgaaccaatcccaaagcaccccggagatcgattccgccacagctttacaacaatatttcgatcgcatccccgtcagttccatctccggcatcaaatccgccctcggtaaccccatctccttcactattttcctttgcacgcaacctgtttgttttaatgccaattcagaaatcccttctctggatgcaataagggttatgtttaagaagaatgtggattgtgctctcatagccgatgtcaccagcgagggatttcccgatcggttcattggctttgtccatctctcaagcttgcttctatggtgtattcaggagattgggaagttggaggatgaaatcaaatgcattcaagcagaggaaactcaagagaatgttcgtgggattctgagttttcttgaacagaatccacatattggccaaaccaaggtgggtgagttagggaagacatttttgtgggatccttatttcccagtagggatgagggatacacttttccatgttctgctattgctttcaaagcatcggttgcaggctgttcctgtagttcagctctgcaattctcacgtcgttggtttcattacaaaggtactttctactgcttcataa

SEQ ID NO: 78 - protein, 743 aa, Cucurbita maxima
MDSCFLSFKPNSHLCLNQNGFYGEKVRGSLDNNALFNRLAKSLKSENKALKLTSNGAASVVATLNITKEAASIQAPPFHTVKVNPKNVASIILGGGAGTHLFPLTRRLATPAVPVGGCYRLIDIPMSNCLNSGINKIFVLTQFNSASLNRHISRTYFGNGVNFGEGFVEVLAATQTPGKSGMYWFQGTADAVRQFIWVFEDAKNRNVENILILAGDHMYRMDYMDFVQNHVDRNADISISCAPVDDSRASDYGLVKLDSRGRIIQFSEKPKGAHLNAMRVDTTSLGLSREEALKSPYIASMGVYVFKTDVLLNLLKWRYPTSNDFGSEIIPAAVKEHNVQAFVFRDYWEDIGTIRTFYEANLALTEEFPKFEFYDPKTPFYTSPRFLPPTKIDKCQIVDAIISHGCFLRECSIQHSVIGERSRLDYGVELQDTIMMGADNYQTELEIAALLAEGKVPIGIGQNTKIRNCIIDKNVKIGKDVIIMNKEGVEEADRPEHGFYVRSGITIIMEKATIKDGTKPEEEEEEEEEEDATGSQKMAQPESNQSQSTPEIDSATALQQYFDRIPVSSISGIKSALGNPISFTIFLCTQPVCFNANSEIPSLDAIRVMFKKNVDCALIADVTSEGFPDRFIGFVHLSSLLLWCIQEIGKLEDEIKCIQAEETQENVRGILSFLEQNPHIGQTKVGELGKTFLWDPYFPVGMRDTLFHVLLLLSKHRLQAVPVVQLCNSHVVGFITKVLSTAS

SEQ ID NO: 79 - DNA, 4169 bp, Cucurbita pepo
  source 1..4169: mol_type = genomic DNA
aaatatgaagggtaaaatcgacactgagaccacatcaaatatcagtggcaaagcggttccctcattcggcagttgagctgcgagctaatctctagttttttgtttttcccggccactgtctgatcggtgaagaaaccggatttcgctttgtgaatttggattgattctccaatggcgtcttctattgctgccggtggagttctcagagtttcgacgacgtcggggtcttcgtcgtcgaacggcggttctaaccgtgtaaggggaagcagtgttcgaagcctctcgttcggtgcgtctctcatttctggtgacaagattgattttagagcttctgctttgggatcacgacgtttttctggtggtagagcggctcctttgatcgtctctccgaaagcggtttctgattccaagaactctcagacttgcctcgatcccgatgctagccgggttggtattcttgtttctttgtgttgttctctgaatgcgttaattgattggaatcgaatagttttattgcctattgtacttgatgcgttcgcggagtttatttcgtttgatttcctgtattgagccgctgcgatttgtttttcgaatggatggtgatttaatggcgtctgttatctgtttttgtgtggttagagtgtgcttgggattattctgggaggtggtgctggaacgcggctctatccgctcactaagaagcgggcgaagcctgctgttcctcttggagcaaactacaggctgattgacattcctgttagcaattgtctcaacagcaacatttccaagatttatgttctaacgcaattcaattctgcgtctcttaatcgtcatctttcccgagcttatgcgagtaacatgggtggttacaagaacgaaggattcgttgaggttcttgcggcccagcagagtccggagaatccaaattggttccaggtgatggaattaaaattaggtttattctttcataattcatgcgaaaattggaatcactgaaaccttccattttgttcagaacattgatcttgtgatatactctgtcttaacacttccaatttcctaatgcattttcttgaagtttgtcttgatctgatatactccgtcttatctcataaatgtttggcattgaaacctgcagggaacagctgatgcggttaggcagtatttgtggctatttgaagaacacaatgttttggagtatctagttcttgctggcgatcatttgtaccgaatggattacgagagatttattcaagcacacagggagactgatgcagatatcactgtagctgcacttcccatggatgaaagccgtgctactgcatttggtctgatgaaaattgatgaagagggacggataattgagtttgcagagaaaccaaaaggagaccaattgaaagcaatgcaggtgaatccaaactccaattctttttatcataataaactgttctcctatgtcttgattcataagatgttcttgttagatgggttagtctaaagcagaaaaaaaaaatcctgttttctttcttacaggttgatactactattttgggtcttgatgatgagagagcgaaagagatgcctttcatagccagcatgggtatttacgttatcagtaaggatgtgatgctgaatcttctaagagaaaaatttcccggagcaaatgattttggtagtgaagttattcctggtgcaacttccattggaatgagggtatggatataatgattttatgcaccataaatataggtcatatctacattgatgaaagtattgtccaaatatatcatagaattcgttagtggaaagtaccaaacaacggtggccttgacaatcgagttgtctgatgcaaactctagtcaatacatgtttaagatcttacatcggtagggtatgaaaaaaacctttccctagtaacacgtttttcctagatcttatatcggctagtggtgagcttgagctgttacaaatattttctgcttcgattaaagaaaataatagaaattaaaagaaaaaaaggcatagtagtagatgaatgagtgctgatcttgaactcatgtcacatttgattactcttggtttgctggagaacgatggcttcagagctcgtaagggctgcgatttctatattaaagatattgatttgactttgtaggtgcaagcttacttgtacgatggctactgggaagatattggtaccattgaagccttttacaatgcaaatctggggatcacaaagaagccagtaccagattttaggtaatatttgattagattggactcgttttctcccatcttttaccaatgaattgttaagttatcatgtgtttatttgatacacgaaacacccaattgcagcttctatgatcgttcgtctccaatctacacccaacctcggtacttgccgccatcgaaaatgctcgatgctgatatcactgacagtgttattggtgagggctgtgtcataaaggtaacttctttacgagcaaagtagctctttgctatgctaagatggtgttgttaccttgtctcgactgcattattccttcccctttccattgttgaaataaccccttttttatttctcctcagaactgcaagattcatcattctgtcgttggcctccgatcttgcatatccgaaggtgcaatcatagaagacacgttattgatgggtgcagattactatgaggtctgtagctaataataaccttgttcatcatgagtttatagctttgagctctcttcaaagtacttttagatacttaaacatcgagaaaatgctgaaaaactacctttaattgttaatgttaccattggaaactaacgtaaagctatatcttagaatagagagccattccaaatgacttgtagttgcagtgttctgtctcactcctttgttgaggaggagataaatttaggggtgtagagcatatactgatttgttgtttccttgtggtggtgctcctcagacggacgccgaccgtaggttattggcggcaaagggcagtgtaccaattggcattggcagaaactcccacatcaagagagctatcattgacaaaaatgctcgcatcggggaaaatgtcaaggtttgtcatgtcaacttttgtttgctttcatttcatcttttacatttatgttattatatcttatcatgcattttcggggtggggtcgatttccattatctttttactctcaatcaaatccactccttccatttagaaggaacaaagacaaatagacaagaaatgagttcanctctcttaagtagttttgacggtgggaataaatctgtttcaaatcagcttcacatggccgcacacgtttggtctttttttttttttaatctatatattaaaaattattgtatgaacttttagttatgtgtctaataattctccatctttacactaattatgttttttttctttcacaacaccaaacacttaaacttgcctaaacctacttgaaatcaactgagtttgataatataaccaccacgaccatgtaaaatattgtttagatttagagatataatcaggtaaacacacgaaaattttaaatttcaaagattagtagtatttattgagaaccagagggtcgatgagttggttatgaatgaattgtgatataattgtctcgacatctggttgagtgtcgatgagttgggtacgaatgaattgtgatataattgattcgacatctgattaagtctcgatgagttgaaatgaattgagatataattttctaactagccgattgatattcatcgcagatcgtgaacggagacggcgtgcaagaagcagcaagggagacagacgggtacttcataaagagtgggatcgtgacagtaatcaaggatgctttgattcccagtggcactgttatctaaagagagaacagaacagaacagaacagagcagacatgtttttcattgaactggtgcctctctctctctctctctctctctctctctctatctatctctcagtctcttcctcagttttggttaccgttccatttggtagtaaatgcagagattagggaagagaatccccactccacagtcccaaccattaggcactgttctacttttaacttatgctgtaaatgttcctcccctcttcttctgcactccatcatttcattaccttccttctttct

SEQ ID NO: 80 - DNA, 1575 bp, Cucurbita pepo
  source 1..1575: mol_type = genomic DNA
atggcgtcttctattgctgccggtggagttctcagagtttcgacgacgtcggggtcttcgtcgtcgaacggcggttctaaccgtgtaaggggaagcagtgttcgaagcctctcgttcggtgcgtctctcatttctggtgacaagattgattttagagcttctgctttgggatcacgacgtttttctggtggtagagcggctcctttgatcgtctctccgaaagcggtttctgattccaagaactctcagacttgcctcgatcccgatgctagccggagtgtgcttgggattattctgggaggtggtgctggaacgcggctctatccgctcactaagaagcgggcgaagcctgctgttcctcttggagcaaactacaggctgattgacattcctgttagcaattgtctcaacagcaacatttccaagatttatgttctaacgcaattcaattctgcgtctcttaatcgtcatctttcccgagcttatgcgagtaacatgggtggttacaagaacgaaggattcgttgaggttcttgcggcccagcagagtccggagaatccaaattggttccagggaacagctgatgcggttaggcagtatttgtggctatttgaagaacacaatgttttggagtatctagttcttgctggcgatcatttgtaccgaatggattacgagagatttattcaagcacacagggagactgatgcagatatcactgtagctgcacttcccatggatgaaagccgtgctactgcatttggtctgatgaaaattgatgaagagggacggataattgagtttgcagagaaaccaaaaggagaccaattgaaagcaatgcaggttgatactactattttgggtcttgatgatgagagagcgaaagagatgcctttcatagccagcatgggtatttacgttatcagtaaggatgtgatgctgaatcttctaagagaaaaatttcccggagcaaatgattttggtagtgaagttattcctggtgcaacttccattggaatgagggtgcaagcttacttgtacgatggctactgggaagatattggtaccattgaagccttttacaatgcaaatctggggatcacaaagaagccagtaccagattttagcttctatgatcgttcgtctccaatctacacccaacctcggtacttgccgccatcgaaaatgctcgatgctgatatcactgacagtgttattggtgagggctgtgtcataaagaactgcaagattcatcattctgtcgttggcctccgatcttgcatatccgaaggtgcaatcatagaagacacgttattgatgggtgcagattactatgagacggacgccgaccgtaggttattggcggcaaagggcagtgtaccaattggcattggcagaaactcccacatcaagagagctatcattgacaaaaatgctcgcatcggggaaaatgtcaagatcgtgaacggagacggcgtgcaagaagcagcaagggagacagacgggtacttcataaagagtgggatcgtgacagtaatcaaggatgctttgattcccagtggcactgttatctaa

SEQ ID NO: 81 - protein, 524 aa, Cucurbita pepo; identical to SEQ ID NO: 66

SEQ ID NO: 82 - DNA, 8082 bp, Cucurbita pepo
  source 1..8082: mol_type = genomic DNA
cttcaggtacacttccttttcccaacatttatatagcttagcgatggagatgggatttagtattttcaaattttcaatcacttgggtatttctctctgttggattatatttctctgttttggctgtgtaatccaataagatcgatcaatttgcaggctctgtctcatggagggcaccaccccacacggccactggcagggcatgaacatgccaccatagcctacagcagcgtaagcaagggcaccggcgtgttcatcccaaggtccctgcagcctgcaagaaaacagaggcgtggaagaaacaataacatgaagccaaaccgacagtttcaaatcaatattgatgtgaactcacaagcatcttccaacaaccatttgatgcgtcctaaaaagggttaaggattcgaagaatttgaggaaaagggtatcagaattcagattaaagaaataattctgtcaccaagtattaagttaatggtgatggctgtacatggaagaagaagaagaagaagaagccatagtattagactgttaatcactgggttttaaattggtttatgtggttcattggaaggttaattaacctaaggtttgtaagcctcctcccctcccttagtgaaaaatttatggttgttctaataataataatatgccactttcattctctctctgttttgttttggagagagagagagagagagagagagagagaggtgccttgaaacatgctctgttttgttgatctgaatcacaaacagaatcatcagactctgcaaaagctgagatgagttgaattcatgttttgaagaatatggccttaactggcatccctgcaatggcacatgaggaggctttcatgcttcaatagctgtctggatctttgttgcagagaaagctaaagttctctaaaacatattcacactgaaacagaggagagagagaaagagatttctgaagctttttggattctgtaattatttttttgaatctgaagtacaccaaaatgatgtgtgtattatgagggaaggattagtgaaaatggctttcaaattatactcactaactagaaaaaaaaaactcttatttaagttttttaagttgggttagttcgaattgttcttattaagttttatttgtaataaatattttgaaacgtgacttaagcttagggaagaactacaattaaacccttattagtgggtaggtctttgaaatcttgctttatagggctaaagattgaatttctatatatttaatttgaaaagtggaggaaacccataaaaagtatatgtcttctaattttggtgaaaatgaaagtcaagaataatatattacaatttataatgcattgaatatataaccaaatttcaaatactctttaaaatatcagtcatatcatcacctttaatcaatcaattttcctagatgttgacataaccccaccactattattttatgttactataattttttttttttttttgtttattttctttataaattaattatcgacccaattatttttcattaatatttttcttgtttttttttttctttttcttttcggaatttgaaatgttaatctaggtgggatggaaaagatgagagaatcaattttaatgaaaatatttggattttttttaatcttttcattaaaatattttttttcttaagtcggatagtctcgtaaaaagataatctttcctttaattgtttgtcttttaaagatatgatctcccagtagctggagagtctcctatgtatccaatacaataaaatcaagtgacgattttgtctccaccacaagaaaattatctctacaagataacggccttgtntttttttttttttttttttttttttttttttttttgttttttctttgttttaaatacataatttagtggttgattccatgccattggagtgaaaggggattaacaccgtcctacttttaagggataagagcatgtcttgtggtaccactggggacaagaattctccagtccccaagcatatccttcacgctcaagggtttctttttcatatcacttcttaaggttattatactaatattgtggataataaacacttcaaatagaaagaccataattagtctatttctttggtgaagttgaagattcctcgatgtcgttttcgaagtggagggatttaaattttagataagattctttgtatggacttatcctagttaagataagcactaaaaatccccccttttttttatggtaccattcatctcctctggcagattaggcaatatctatatataatctccttccttcgtcatagcttatttcgacttattaaagattgaagttttttaaaagggtcggtctcaattttgtgattttgatttagctaaatctcaaagatgcatggtctaaggaaatgacttgaaaggtctaatcaattatgaacaaattttggaataaaagaagaaaatatacgatttgggtaattcaattgaacacgttttttatttaaagattcgttcatttaaataagaacgcatttttaagagaagtgtttttcaaccgaaatattcataagttggcgatgttcataccaaccaaaaagtaaaaagtactttaacataagagttcaactagtgggttgggaggattccaacatgccataactttgagtgatttttaaatagaaagaggggaataacttgggtatcattttgtaaagcaatctctaattttttttaggaaatttttggaactaaatttaagatttattaaaattcaacatttgaaattacataactaaaattaaacaagatgtcggtactgaaatactattataacaaaaaagttatgcatgaaaatgataacatcctaaatcaaccatagaacttggaaataaattcaaattagttgaaaatttaatggaaatgaataagataggttttataattcttgaattaaagtttgctttccttcactttcttgggctgtgattcttagaagggaagtatgtagctacatggcatactgaaggcataaaaaacttcccaagtgagccacccagcaatcctaatggcactaataaggaggttgtgctaaatgatgggcagcaaacttatggatcttggtgcttgtgaatgtcattgacaagcgaccctggaagaattttggaacctcatcatgctagccattattctacctactttgatgtcctactttgtcttaccagaatgccatcgactatcttcatcaaggcaaaaagatgggtaggctttaggaaatcattttcgtggttaccaaacttataacatgagaagagaggaagcactcttttttctatgatcagctggttgcttagaccaatgttttctaaacctcaaggggcactaaagcacaatagccctccacgaggcacaagcaaaaagtgcaggcttttttacgagacaaagaaaagaatagaaaaatgtatatagatttttaatatatgaacataaatagagattttattcgagatcataagaatattcatgcattccctcattaaaaaataaaaataaaaatacaaactcacaccaggctttagaacagaatgatcataatcttagagacagagcaaagaggggtcatttacctcgatatgttatatgccctcagcttggaatggtcgttgtgctgaagaagaatcacatccatagatatattttgttgttacatctcctctgcattcttattaaacattaacgaaagaaataatacaaatcaataaaggatcgacatgttcatcaacgagggataaacatgtttattatataaccaaaccatcttcaattactgcattttttgagatgatggtaatgccagaacgtatgtagaagccttctgaagatctatccgcctcttgtacgccctgcaatatgtttccagtaatacataattcattcatttcgaggtcaattgaaatctcattggttttacgaatgcaacaaatcccttttttactctatggtaacggtacctctgaattggcaatgaccacgttctttccaattctagcgttcttgtcaataatgcagtccctgcacataatatttatatcaacaatgaggaatgagaaaagcaggctcaaaaatatgtttggttgtggcgttcatactttatttttgaattttctcctattcctattggtactcttccttcagctaacaaagatgcaacttcagcttcagtttcatagtaatctgctccaagcatcacagtgtcctgcattttgcatttcatcacaaatcactccgttaagaaaaaacttatgttttgcaaaggactgaattcaactccttagtttgattatcacttgcaaaagttccatgagatgcatactttcaagtgcacgttagagttaattctggaccgaatgccgatgacactgtgctctataaagcaactgcttaagaagcttccatgtgagatgatagaatctacgatctgcaaaggacccaaatattatctcaacataaagagcaaagaaaaattgtcacaccagaaaaatgctcagccgttgttaataactggtttcttttctccaatgccaaatgcagtgttattttagggccaaatacagcatatttcgttaaaaaatggaggtttttattagagtttatgattttataatagagatttagacgaccgcagaatactactgaccttgctctggtcaatctttgagggtggcaagtttcttcttgaagtaaacatcggttttgcagcatcatagaagctaaaccttggtggctgaaaggtatatgcaaacaagcaagaataacaataaaaaacggtcaaactacaattcgaataacttgagaaaagtcctaaaggatgtgagagagagagagagagaatagcaaattttaaatggaaagtgtttagttgcctaacttgctctgtcagggcaaggttcgcttcaaagaaggatctaattgtcccaatgtcttcccagtaatcattgaagagataagcctttataaagtattctttggctgaggcagggattatctcagatccaaagtcattagcagttggaaagcgccatctgacttgaacaaattcaagcaaagtctatacttcgtgttgaacaaaatcctttgagtttgtataaaattactagattcataaatagcattagatgcacaataccttaaaagatttagaagtatctccttcttaaagacgtacactcccatggaagcaatatatggcttcttctgagcctcttcttttgacagtcccaaaatggttgtatctaccgccttcaatatgaatggaaatgataaagtttaggttcatcagaacaatcattttaagttcctccacccatttgaaatttctagtgaaggaaattttgaatgttaccatggccttcaggtcctctcctttaggcttttcactgaatgaaagtatccttcctttgtcgtctatcttcattagaccaaagtctgaggctcggctataatacaaggtaatgagatttttaaaaatttgcaggagtataattagacactcttctgctttcgattaaaaaaacataaatttgaagaaaagatgcactttcctttcgttgcacatttctgtaattttttgaaccttaagacttagatactcgagtgtttgcgaacagaaaaaggtttgaactgattgcatgatcaagatctgtggggccttcaggatcaatgattttttagctaaccgtccgaaagagaaacatcaagttgtcactcacctatcatccattggaagacaagaaagagtgatatctgcaccactttgcctatgattctgccaatgtaaagaagatacattaaaactgaatgtaacgacagatgaaaaatggtttcttaagtgcatttacatccttacctgaacaaaatccatgtagtccattctgtacaagtgatctccagacagaatcaacacatcttcaatattcttgcttctggcgtcctgcagcaggcaaggcttattactatctcagtccttaggtgaaggcctgttttctatgtcagatatgaccccaaggaaagttaatacgtatttaacccaatttttcctttcatcacttactgtatatggaacaaccaaatgtcacaatcatttctttatatgcttctccatatcaatatcttacatcattatgttattagataaggtttcttgtgtacctcaaagagccagtggaattgtcgtacggcatctgctgtcccttgaaaccaatttttacctgccacccctggcgtttgggtggctgcaagaacctgatatgtagctgaaaccttaagtcatattttgctttgggaatataattgtggatatatgatccgcttaattcttgagggacatgttttaagtgaaggaaacgtacctcaacaaagccatctccaaaggtgactccaccactgtaattgtaagctcgagcaagatgcctattcaatgacgctgaattgaactgagtgaggatataaactttgttgattccactgttgatacagttgctcattggcacgtcaatcagcctatacgctcctgcaattggaacctgtaacggaaaatcataagaaagcacagagcagccaatataacaaaatcacatatggatatatgaaaataaaatgtcactgattccaatcatctaaactaaatgaacctgaagagggaaaaataactggacctcaattgatttcacagtggcttgcaattacaatacttcaatgataaatagcagtaagaaaaattgagtttagcagggaaaacttacagcaggtttggcacgccgcttggtgagggggaatagacgagttccagctcctcctcccagaatgattgcaaccacagtccgagggtcccttttttccgtatctatttctctcaactaaacaaagtcacaagcagcaacacattaactgttgtcccagatcattttgagagtgaattatacacaatatttccctactagtaagagatagaaagcagggtgagtttctgatatcactcctcccaattaatcaatacttttgaaggggtctcacttaggaatgggggaaacaaaagattgtgacggtctaaaaattattcagtccgttgattcacttcccaatggtatattttgagtttgttcaaatatatataaatatatacacaggtcaggttaagtccaaattgcagcacgttgttagattcaactggaatcagaaggcgtagagtcctgaacaaagaatcgaatgatctaaggtcatagtagatggtaaccacataaatcgcactggttactaattctaaaggtagaaggctagacattagcatcctaattagcagtcttctctaaagttcaaaccatatcttggagacttacgccctaggaaaatttatcgaaatacagagacagaccatcaccaacacggagccattgcatttcaaaatgtggacaaacaaactaaatcaatgcgaagcaacgaaggtgtagtttcaaatctccgcaaaattaccgaaacaacaaagttcacaccgcaataaaaacaaaacaaatctggacaagttcaaactctaatgttccatatcgtatatagaagattaaaatacttctcagcagagttctcaacattaccgcagcgtcgccaatggctacattagtggtaagagacatacaaacacgtcgcccaacactcttagcagcagcgcctactcgtttcgactccttgaccttgagcttcattcccatgaactcgccattggcaaactccacagacctcaacttcctcccgaccaccaccgcagccgcacgtgcatcccgcacatgcgcggcggccgataaggcaatccgacagtcggcagagaccgccatttttcgatatctcggaaatggaggaccgattcgaaactgctagaagcttagacgttgcagaagaaagttacgcatcgagtgaggagtgctcacatggactgcatgggacctggaaagaacaggaccatgttatcctttatgaatttcaaccactaattcagattggcggtagatggaaactccatcacacccgctcgtt

SEQ ID NO: 83 - DNA, 1581 bp, Cucurbita pepo
  source 1..1581: mol_type = genomic DNA
atggcggtctctgccgactgtcggattgccttatcggccgccgcgcatgtgcgggatgcacgtgcggctgcggtggtggtcgggaggaagttgaggtctgtggagtttgccaatggcgagttcatgggaatgaagctcaaggtcaaggagtcgaaacgagtaggcgctgctgctaagagtgttgggcgacgtgtttgtatgtctcttaccactaatgtagccattggcgacgctgcgttgagagaaatagatacggaaaaaagggaccctcggactgtggttgcaatcattctgggaggaggagctggaactcgtctattccccctcaccaagcggcgtgccaaacctgctgttccaattgcaggagcgtataggctgattgacgtgccaatgagcaactgtatcaacagtggaatcaacaaagtttatatcctcactcagttcaattcagcgtcattgaataggcatcttgctcgagcttacaattacagtggtggagtcacctttggagatggctttgttgaggttcttgcagccacccaaacgccaggggtggcaggtaaaaattggtttcaagggacagcagatgccgtacgacaattccactggctctttgaggacgccagaagcaagaatattgaagatgtgttgattctgtctggagatcacttgtacagaatggactacatggattttgttcagaatcataggcaaagtggtgcagatatcactctttcttgtcttccaatggatgatagccgagcctcagactttggtctaatgaagatagacgacaaaggaaggatactttcattcagtgaaaagcctaaaggagaggacctgaaggccatggcggtagatacaaccattttgggactgtcaaaagaagaggctcagaagaagccatatattgcttccatgggagtgtacgtctttaagaaggagatacttctaaatcttttaagatggcgctttccaactgctaatgactttggatctgagataatccctgcctcagccaaagaatactttataaaggcttatctcttcaatgattactgggaagacattgggacaattagatccttctttgaagcgaaccttgccctgacagagcaaccaccaaggtttagcttctatgatgctgcaaaaccgatgtttacttcaagaagaaacttgccaccctcaaagattgaccagagcaagatcgtagattctatcatctcacatggaagcttcttaagcagttgctttatagagcacagtgtcatcggcattcggtccagaattaactctaacgtgcacttgaaagacactgtgatgcttggagcagattactatgaaactgaagctgaagttgcatctttgttagctgaaggaagagtaccaataggaataggagaaaattcaaaaataaaggactgcattattgacaagaacgctagaattggaaagaacgtggtcattgccaattcagagggcgtacaagaggcggatagatcttcagaaggcttctacatacgttctggcattaccatcatctcaaaaaatgcagtaattgaagatggtttggttatataa

SEQ ID NO: 84 - protein, 526 aa, Cucurbita pepo
MAVSADCRIALSAAAHVRDARAAAVVVGRKLRSVEFANGEFMGMKLKVKESKRVGAAAKSVGRRVCMSLTTNVAIGDAALREIDTEKRDPRTVVAIILGGGAGTRLFPLTKRRAKPAVPIAGAYRLIDVPMSNCINSGINKVYILTQFNSASLNRHLARAYNYSGGVTFGDGFVEVLAATQTPGVAGKNWFQGTADAVRQFHWLFEDARSKNIEDVLILSGDHLYRMDYMDFVQNHRQSGADITLSCLPMDDSRASDFGLMKIDDKGRILSFSEKPKGEDLKAMAVDTTILGLSKEEAQKKPYIASMGVYVFKKEILLNLLRWRFPTANDFGSEIIPASAKEYFIKAYLFNDYWEDIGTIRSFFEANLALTEQPPRFSFYDAAKPMFTSRRNLPPSKIDQSKIVDSIISHGSFLSSCFIEHSVIGIRSRINSNVHLKDTVMLGADYYETEAEVASLLAEGRVPIGIGENSKIKDCIIDKNARIGKNVVIANSEGVQEADRSSEGFYIRSGITIISKNAVIEDGLVI

SEQ ID NO: 85 - DNA, 4957 bp, Cucurbita pepo
  source 1..4957: mol_type = genomic DNA
tattttctttatttgttataaacaaatcactacaagaggccaagcgagagctgacgagtaggtcccaattgcaatagccgtattcccgccgtgtcaaagtcccaaggcgccctctctctctgcactcgccattattgaaagccaagatcgcccgacgatcctctggcggctcaaaaccaaatttaacgacgctagttcagagttttcacattcctcactaggatcttcatccttcgatctttgatctattcccgcctttcttctttactctgttcattcgaagctgttccctctacatttctcttacaacactctctttacagacagggctcctccagcgtttgatcctttttcctgcgttgtacttcaatctccttctcagagctttggtttccctctgatctaggcttttgagcgtgctccggtctcgacgattgcgttggaaaggatcggagatctttctgagcttcttctgcactgtggtcgacttaacatattcgggcaagctactcggagatcagatttttctcttgatctgtagttgatttgacaatggattcgtttggtgtgaccttgaaggccaatacgatgccatttagagccagcagacagtgtggtaggaaggtgcaatgctccgggttctggggagagggtattgagagaagcggaaagtgcaaacagatacagatgaatgcttactcgcggaagaattctaattcttcttcccatagcagggctagaaagcttacagcgggagttgctcattctgttctcatgtcagaagttagtgaggaaacagcggtaagtggatttagtactttgatattcaaaagtacatgaacttttatcggaacttttgactccaaattttaccccagactttacaggctcctatatttgagactcctagagctgatccgaaaaagattgcttcaatcatactgggtggaggtgccggaactcgcttgtttcctcttaccagccagcgcgccaaaccagcggtaaccacctctaccaaattgcttatataagaataatttcacaaacttggagagcgtttttcattggggagatgtatttagatggttgtagtagatacttgataatagaagctattgatccaacccaggagaaaaattggtgcatgaatttaagcaaataatttttacttgcaggttccaattggaggttgttatagattgatcgacgtgcccatgagtaattgcattaatagcggcatcgaaaagatattcgtcctaacgcagtttaattcgttttctctaaatcgtcaccttgctcgtatttacaattttggtaatggagtgaattttggcgatggattcgtggaggtatgatgcctgtacttcccatttttacaatctgcccctgctttgcaattgttttgattagatgaacccggaagttttcttatgctcattctctggattaatgtttgagaaacaggttctagctgccactcaaacttcgggtgaaactggaaaaaaatggttccaaggaactgccgatgctgtcaggcaatttatatggttatttgaggtgggtgcctaaaatctcctgagcatgattggaattttcagtcaaattttttttatgtataatgaacgttttatccattgggtttctgcaggatgctaagaccaaaaatgttgagcacactctaattttgtctggcgatcacctttaccggatggactacatggattttgtgcaggtataacgtgaagctaactagtttgtacttgtatttgtatcgttgtgctcattttttcgtgattcaatgaggatttctgatgacttcgtacttgcagagacatattgacacaaatgcagatattacagtttcatgcatacccatggatgtcaggtaggcatcttttttcatcgttatttccatggataaagtgttctttaaaaatttagattttcattaatgctttgttattttgcagccgagcatcagattacggtctcatgaaaatagacgacacagggcgtattttacattttgctgagaaaccaaagggatctgacctggaagcaatggttggtaacattgatgcaatttgtttggtttgtaattagcaatatctttgtctgatcttgttttctatccattatatattgtattgcagcaagttgacactacagttctaggactttcagaccaagatgctagaaaaaatccttatattgcatcaatgggagtctacgtattcagaactgatcttctattaaagcttttgacatggacttatccttcatgcaacgacttcggctcagaaattattccatcggctgttaaagactacaaagttcaagtgagatcccttttgagaaattttaatctttcattttttccttatgcttgaattcagaataggaagactttcgatgataatagaactaagtttcccaatgcatccatattgttgtaggcatatctattcaatgactactgggaggacattggaaccgtgaagtcattttttgatgccaacttagcactaacggagcaggtatgtatcccatcgattgtctcctttcttttcctttgtatgggaccaagaatcatctccagctttcttgcttatttagttatctgagttaaatatttatatagaagataagtaccttaactggcccgtgaatttttcaagcaacgccctcccccgcatcagaaacaacacatgatcaagtgtcattaagctgatggctttagtgtctttcatgcagcccccaaagtttgagttctatgatccgaagactccattttatacatcacctagattcttgcccccttctaaagttgaaaaaagcagggtaagacatcaaactcgagtttgctctctgcatcatccgtttgattcttgtatcttaaacgaagttgcttttgcagattgtagatgcaataatatctcatggttgcttcctccgagagtgtagcgttgaacactctattgtcggtgtgcgctctcgattggagtacggtgtggagcttaaggtgagccagggttttcttttgaattttcccctccctttcggtactgtaggaacgtacaagaagaatgcaatggcagaagatggttatagatgctgcaatcttttttttccacagattaaattcttactattattgcaagagattctgccatgctgatatgtagcatcttagcctgaaaaaagataatcaaatttggatttgttggttttgcgttctaggatactatgatgatgggtgcggactactaccagactgaatctgaaatcgcatctctgcttgccgaaggaaagatcccaatcggcattggagagaacaccaaaatcaggtaatattcagctaccaatacagagcaaaagctatctgcaatctcttattagttaaatataaccgttactttatcatccgactgtttaccataaacattatttgttcatgggtttggtctgtttctaatttacatttatcttttccgcaggaattgcataattgacaagaatgccaagatcggaaaaaatgtagttattgctaatactgatgtaagtatgatttttgtgttcttaagggatatttggttcacaatttaacaagttctgtttcatccatgtttgtatttgtacaaagaaataaccaataaaaggttaggtttggttcttgggtttcataacctgggtggtttcatatgtgaaggtttatagaacatgagttcatgcaattcggtaggaaggggaaaattaacactttctaagaatttgcaaactctatctaaaccacataaacccgtgatgttatttggtcatagtacctaactagtgtgtggttgttaacttttcagggcgttcaagaagcagatagaccagaagaagggttttatataaggtccggtatcacagtcaccttgaagaatgcgacaataaaagatggaaccatcatttaaatccaaagagaaagctgagagttgaaaggaagcaactgctcgaggctttcaaccgacaatgctcggcaacgagtaattggcgcatgacttagcttcatgtaaaatgttagcttcttttcttatatgtaaaaagttgaaaggcaacacattcctgagtaagagaacagctttttatttcaatccatatattattagagactacaatattcaaagcgatttagcattagtttttggatgagcaatcctttctctgtgagggagttgtttattcttagccccatttttgttcaatttccaggactgtggctgcctccctttaaggcctccccttgtgtcttatgtttctgaggttggcatgcttcttccttattttgatcatctttgcagactcctgagatatcaaattagcagtggaggggtcctcactaaataccacttcatacccatccccataagaacccatcccttcaagtagaagttgccagaacactgcacccgtagctgctccgccgcccttagcggatgaatacacagctgagtaaatagcatggtaaagtcggtcccttttatcagcgccaccgtcctttttagactttccaaactcagcaaagagaactggcttgtggagaacgttctgtgcatcttgggtgtgttcattgagccaagtgttcaggaatgagagctgatcctcaaaacttgaaccagataaccttcatcaaatcaccacgttcatttcagctaataatggattgaagatctcagaaaagcagcaaatgggagaagggttttgttcttaccattcgtcaggatacgagtggactgtggcaaagtcgatttcgggtatctgattgtttgcaatgaaatcggttcccacttggaagttaggattcccttgataccttgcttggttatagaatccttccaaaccaacttctaagaggtgtttcccatcaattgatttcaagtatgcgcccatttctttaatccatgcctgcccatca

SEQ ID NO: 86 - DNA, 1602 bp, Cucurbita pepo
  source 1..1602: mol_type = genomic DNA
atggattcgtttggtgtgaccttgaaggccaatacgatgccatttagagccagcagacagtgtggtaggaaggtgcaatgctccgggttctggggagagggtattgagagaagcggaaagtgcaaacagatacagatgaatgcttactcgcggaagaattctaattcttcttcccatagcagggctagaaagcttacagcgggagttgctcattctgttctcatgtcagaagttagtgaggaaacagcgactttacaggctcctatatttgagactcctagagctgatccgaaaaagattgcttcaatcatactgggtggaggtgccggaactcgcttgtttcctcttaccagccagcgcgccaaaccagcggttccaattggaggttgttatagattgatcgacgtgcccatgagtaattgcattaatagcggcatcgaaaagatattcgtcctaacgcagtttaattcgttttctctaaatcgtcaccttgctcgtatttacaattttggtaatggagtgaattttggcgatggattcgtggaggttctagctgccactcaaacttcgggtgaaactggaaaaaaatggttccaaggaactgccgatgctgtcaggcaatttatatggttatttgaggatgctaagaccaaaaatgttgagcacactctaattttgtctggcgatcacctttaccggatggactacatggattttgtgcagagacatattgacacaaatgcagatattacagtttcatgcatacccatggatgtcagccgagcatcagattacggtctcatgaaaatagacgacacagggcgtattttacattttgctgagaaaccaaagggatctgacctggaagcaatgcaagttgacactacagttctaggactttcagaccaagatgctagaaaaaatccttatattgcatcaatgggagtctacgtattcagaactgatcttctattaaagcttttgacatggacttatccttcatgcaacgacttcggctcagaaattattccatcggctgttaaagactacaaagttcaagcatatctattcaatgactactgggaggacattggaaccgtgaagtcattttttgatgccaacttagcactaacggagcagcccccaaagtttgagttctatgatccgaagactccattttatacatcacctagattcttgcccccttctaaagttgaaaaaagcaggattgtagatgcaataatatctcatggttgcttcctccgagagtgtagcgttgaacactctattgtcggtgtgcgctctcgattggagtacggtgtggagcttaaggatactatgatgatgggtgcggactactaccagactgaatctgaaatcgcatctctgcttgccgaaggaaagatcccaatcggcattggagagaacaccaaaatcaggaattgcataattgacaagaatgccaagatcggaaaaaatgtagttattgctaatactgatggcgttcaagaagcagatagaccagaagaagggttttatataaggtccggtatcacagtcaccttgaagaatgcgacaataaaagatggaaccatcatttaa

SEQ ID NO: 87 - protein, 533 aa, Cucurbita pepo
MDSFGVTLKANTMPFRASRQCGRKVQCSGFWGEGIERSGKCKQIQMNAYSRKNSNSSSHSRARKLTAGVAHSVLMSEVSEETATLQAPIFETPRADPKKIASIILGGGAGTRLFPLTSQRAKPAVPIGGCYRLIDVPMSNCINSGIEKIFVLTQFNSFSLNRHLARIYNFGNGVNFGDGFVEVLAATQTSGETGKKWFQGTADAVRQFIWLFEDAKTKNVEHTLILSGDHLYRMDYMDFVQRHIDTNADITVSCIPMDVSRASDYGLMKIDDTGRILHFAEKPKGSDLEAMQVDTTVLGLSDQDARKNPYIASMGVYVFRTDLLLKLLTWTYPSCNDFGSEIIPSAVKDYKVQAYLFNDYWEDIGTVKSFFDANLALTEQPPKFEFYDPKTPFYTSPRFLPPSKVEKSRIVDAIISHGCFLRECSVEHSIVGVRSRLEYGVELKDTMMMGADYYQTESEIASLLAEGKIPIGIGENTKIRNCIIDKNAKIGKNVVIANTDGVQEADRPEEGFYIRSGITVTLKNATIKDGTII

SEQ ID NO: 88 - DNA, 4933 bp, Cucurbita pepo
  source 1..4933: mol_type = genomic DNA
agttgtgttcatcacttaaaagaccattagcatttgtatctgtgaactttaatcctcacaaacattggtttgtacttttgattccccgtttgcataaggtgccattaggatactcctttgttagcgtagtttcactattgccaacaacctttcaaaggtcttgtttttgtaaataggacatcatgcatgttgctcgcgagttgtagttgttgttgagtttttttattcctccaatgatttagaaatcacccatcgtgttacctatataccttagtaatattaaacaggcttttccagcacaaatttgcagagtcacaaactactcacgacacaaaagaaattcactcactaataatctcaagaaattgtttctgatgtagaatatcatagagacaaaactgcaaccacaaagcatagtaagaaattccaagagatcagataacttgcatcaacctcgttctgattccagattgttaaacattagtagggtgaaggaatttcttaagatgaaaactttattagggtgaaggaatttcttaagatgaaaaccttggtagggtgaagggatttcttaagattgagaggaaagaagaagcactaaataagacggaataaataaacatttaatattattcaatttttttttatttaaattaagatctttagcttttgtaaaacaatccatctttcaatgtactttgtgcatttatttatttatttgcatgaaatttgttactattatttaaccaaattcaattaaaataaaattaacacaaacttgtaaaaaaaaaaaaaaaaaaaaaaaanatctaacgtagtgcataatacataaacatatatttcttttatacataacataacctactaatctggctgagtagccactcttattgaagagaatttcactttaagacttagaatctctagtttagaactcccctatgtggatgctttccttcttctccttgttctctcagccacaatgctgcaaacgccattcttcacctgccacaccccaggcagtctcctgaaaacttcacaccatttttccttctctaacatgtcttccaaaccgaggtattcgatctgttcatataactgtgccatcaccgatcgttgccttttccagtataatggtgattcccgaccgaatgtagaatccctgttcaggccgatctgcttcttgaacgccctgaaattgacagagatacagattaagaacatacacaatgctctggggttttcaggattgaatgcatattgttgagctgggttggatatgcttacctctttattcataatgataacatctttcccgattttcgcgttcttatcgattatacagttcctgagacagaccagccaccatcaatgtagatctatgttgtttaacaagcttctaaatgtttttgtttacttacttgattttcgtgtttcgtccaatccctataggaacttttccctctgctagcagagcagcaatttcgggttcggtttggtaattgtctgcgcccatcattatagtgtcctgtacaaatgaagacaacaacaacctgtgttggttacaaatccaggtatttgggctttgtctgaattataagctttttgatttcaccttaagttcgacaccgtagtctaatcttgaccgctcaccgactatcgaatgctggacattgcattctctcagaaagcatccatgtgagattattgcatcgacaatctgcaaaacaccaactgctgcttaagagcagagtcctaaagataagcttaaaatcggactggttttatcgacatacctgacatttgtcgatcttggttggcggtaggaatcgaggagatgtatagaaaggtgtcttggggtcatagaattcaaacttaggaaactgcaagaaagtgaggatttgataatcaatatcaactcaagtgaagcaattgaatacattgcttgaagtagagaacaatggcattcagttaattccacagttcggattcttaagatcccgatcgcaagaaacgaaacgcacctcctccgtgagggccaagtttgcatcgtagaaggtctttattgttccaatgtcctcccaatagtctctaaatatatatgcctttagaataagacagcaaaaaggaagaaagaaacagaagttacactcacacatgatatcaatgtgacatatgagggattcagataggttttatgtctagcactcggactcgattccatgtgacgttaatataaaggatcattaccccaaaactatattcctttactagttcatgatataaatccaagattatagaacatgggtgcttcaagtttcttagctatgtcgattttcgtaggttccgactattattaccttgaacaaatttatgtaatgtcgaaacgttaagagttgatcgaaagtgatcttacttggacattatgctccttcacagctgcaggaatgatttcagaaccgaagtcattagatgtaggatatctccccttcaaaaggtttagcaaaatatcggttttgaaaacataaactcccatcgacgcaatgtaaggagacatcaacgactcctcccgagacaaacctaatgaagttgtgtctactcgctgcagaaaaccgttcgattaggatggttatgtcccttgaatattaggaatgcagtttgaaaaacaatagtgattgaagagaaggtttagtacacatgaacatttttcagctatgaaggattgtcttaatggaaaaggcaaccattctatgtttactttagttcttaccattgcattcaaattggcaccctttggcttttcagcaaactgtataatccgacctctactatccagtttcaccaatccgtagtctgatgcacggctgcaaagtagtcgatagaaaagaaataagactttggttctaaaattccgaaagaacgaatagataaatttcctatgatcgacaagaacaacatacaaagttgttgtgagcctcgaaggaaagggtgtattttgttcgagggctccaaagaaggagtcgagcctcgattaaagggaggttgttcaagggctccataggccttaggctctatggtgtactttgttcgaggggagagttgttgagaattggcgatgatcatgggtttataagcaaagaatacatctccattggtacgaggcctcttggggaaaccaaaagcaaagccatgagatatcataccattgtggaggtccgtgattcctaaccgttaggcactacacctataatctagaagagtttcttttcctattacctaccagagacacaattttaaaagaagaatcataaggtaaagggagtattgataagacctgtcgcccacagctgcgcacgagattgaaatatcagcgtttcgatcaatgtggttctgtatccaaaatcatagcacatacgataagattttcgaaatacggtatgaacaggagctcaaaacgaaactaattacctgcacaaagtccatatagtccattctgtacatgtgatcccctgccaaaattagaatgttctcaacattcctgttcttggcatcctacaagaatcaatatgatactcaacttcatattatcaacaaagtcgagttccaaacagaccaaaacgaaacaaaaaatcatcttatacctcaaacacccaagtaaattgtctcacagcatcagcagttccttggaaccaatacataccagatttccctggtgtttgagtggctgcaagaacctgttcatcaaaagtaagtccaaacgatcatatcgatcactcgtgctacaaagaaaaagatcaactctatctctaagttccttttcgattccgatcttcatatcattaactagtatacctccacaaatccttctccaaaactgacaccatttccaaagtaggttcgtgaaatatgccgattcaacgaagccgagttgaattgagtaagcacaaatatcttgttgatcccactgttgatgcagttgctcattggaatatctataagcctatagcatcctccaactggaacctacccatatgagttcataaaaggatcaaaactaagccctatatatatatatatgaacaagtctagctccttacagcgggtgtagctgatcttctagtaagaggaaacagctgagtcccagctcctcctcccaatatgatggaagcaacattcttaggattcacctttgctttgaaaaacggcggcgcttgaatgctctgcaaaaccaaatccaattcaaagtaaactcaaccaaaacatggctatctacttatgctgtaaactcaccgcagcctgcttggagagattaggcgtggcgacagcataagcaacattaggggagagcttcaaaggtttcttgtcagatttgaaggtcttagctaggcgattaacccaaccattttcattgaaacttcctctaactttctccccatgaaacccattcttcagatcactcgataaatgggtattagctttgaaagaaacagagtaggaatccatcggtcccttcaatagacaaagcatcagaaatagcacaaaatacaatctagtatcagcaaaagccccaatctaagcaaccatttagagtacagatcacacaaacaaaagggaaaaagcaaaaggggaagccgtagaaatgcaagaaaccacctgaaacttgtccagaacattaaagtaatggatacccagaaaagtaaaggacggaaacaagaaacccacgaaagcaaaggagagatcgagcacttacaaaggggaaaggaggcgaaagtagaaggagaagagagtgaaaaacgtgagtgatgatgataatgaagaaggcagtgggggaagaggggttatggttatggttatgggcatgtgagtgtgcgaggggttttcatttcacgaggatttatatactaaacaagaaagtggggaaagcttcattgga

SEQ ID NO: 89 - DNA, 1563 bp, Cucurbita pepo
  source 1..1563: mol_type = genomic DNA
atggattcctactctgtttctttcaaagctaatacccatttatcgagtgatctgaagaatgggtttcatggggagaaagttagaggaagtttcaatgaaaatggttgggttaatcgcctagctaagaccttcaaatctgacaagaaacctttgaagctctcccctaatgttgcttatgctgtcgccacgcctaatctctccaagcaggctgcgagcattcaagcgccgccgtttttcaaagcaaaggtgaatcctaagaatgttgcttccatcatattgggaggaggagctgggactcagctgtttcctcttactagaagatcagctacacccgctgttccagttggaggatgctataggcttatagatattccaatgagcaactgcatcaacagtgggatcaacaagatatttgtgcttactcaattcaactcggcttcgttgaatcggcatatttcacgaacctactttggaaatggtgtcagttttggagaaggatttgtggaggttcttgcagccactcaaacaccagggaaatctggtatgtattggttccaaggaactgctgatgctgtgagacaatttacttgggtgtttgaggatgccaagaacaggaatgttgagaacattctaattttggcaggggatcacatgtacagaatggactatatggactttgtgcagaaccacattgatcgaaacgctgatatttcaatctcgtgcgcagctgtgggcgacagccgtgcatcagactacggattggtgaaactggatagtagaggtcggattatacagtttgctgaaaagccaaagggtgccaatttgaatgcaatgcgagtagacacaacttcattaggtttgtctcgggaggagtcgttgatgtctccttacattgcgtcgatgggagtttatgttttcaaaaccgatattttgctaaaccttttgaaggggagatatcctacatctaatgacttcggttctgaaatcattcctgcagctgtgaaggagcataatgtccaagcatatatatttagagactattgggaggacattggaacaataaagaccttctacgatgcaaacttggccctcacggaggagtttcctaagtttgaattctatgaccccaagacacctttctatacatctcctcgattcctaccgccaaccaagatcgacaaatgtcagattgtcgatgcaataatctcacatggatgctttctgagagaatgcaatgtccagcattcgatagtcggtgagcggtcaagattagactacggtgtcgaacttaaggacactataatgatgggcgcagacaattaccaaaccgaacccgaaattgctgctctgctagcagagggaaaagttcctatagggattggacgaaacacgaaaatcaagaactgtataatcgataagaacgcgaaaatcgggaaagatgttatcattatgaataaagagggcgttcaagaagcagatcggcctgaacagggattctacattcggtcgggaatcaccattatactggaaaaggcaacgatcggtgatggcacagttatatga

SEQ ID NO: 90 - protein, 520 aa, Cucurbita pepo
MDSYSVSFKANTHLSSDLKNGFHGEKVRGSFNENGWVNRLAKTFKSDKKPLKLSPNVAYAVATPNLSKQAASIQAPPFFKAKVNPKNVASIILGGGAGTQLFPLTRRSATPAVPVGGCYRLIDIPMSNCINSGINKIFVLTQFNSASLNRHISRTYFGNGVSFGEGFVEVLAATQTPGKSGMYWFQGTADAVRQFTWVFEDAKNRNVENILILAGDHMYRMDYMDFVQNHIDRNADISISCAAVGDSRASDYGLVKLDSRGRIIQFAEKPKGANLNAMRVDTTSLGLSREESLMSPYIASMGVYVFKTDILLNLLKGRYPTSNDFGSEIIPAAVKEHNVQAYIFRDYWEDIGTIKTFYDANLALTEEFPKFEFYDPKTPFYTSPRFLPPTKIDKCQIVDAIISHGCFLRECNVQHSIVGERSRLDYGVELKDTIMMGADNYQTEPEIAALLAEGKVPIGIGRNTKIKNCIIDKNAKIGKDVIIMNKEGVQEADRPEQGFYIRSGITIILEKATIGDGTVI

SEQ ID NO: 91 - DNA, 4777 bp, Cucumis melo
  source 1..4777: mol_type = genomic DNA
actcaatctcatcaactttacaattttgtttaatttccttttcattttttattggggatacgctcaaacttttccttttcctatattacccctaccaccccccacgtttccatgactaaaccacgtaataaagtggaaattgcaaggatttaatcgtccgtcaaaccacatcaaatatcagtggcgaagcatttctctcgttcgggagtagagctgcagagctgatctctgcttcttttcatttttttccggcgacttgctaatcggagaagattaccggatatcgttgttttggatttgaattgatcgtctaatggcgtcttctatggctgcagttggagttctcagactcccgacgtcgtcgtcgtcgtcgtcttcatcgtccaatggtggttccaaccgtgctaggagaactagccttcgaagcctctcatttggtgcgtctcaaatttctggtgataagattgactttagaggttttggtttgggatcacgacgtgtttctggtggtagagtggctccctcgatcgtttctcctaaagcggtttctgattccaagaactctcagacttgcctcgatcccgatgctagccgggttggtattcttgtttctttgttatattatctgaatgcgtttaattttttggaattatgtagttttgttgtctattgttcttgatgcgttcgttgaatttatttcctttgattttctgtttttgagctgacgtgaatttgtgtgtgaaattgatggtgattaattggcttattttatctgtttttgttgtggttagagtgtgcttgggattattctgggcggtggtgctggaactcggctctatccgcttactaagaaacgagcgaagcctgctgttcctcttggagcgaactataggttgattgatattcctgttagcaactgtctcaacagcaatatttccaagatttatgttctcacgcaattcaattctgcttctcttaatcgtcatctttcccgagcttatgcgagtaatatgggtggttacaagaatgaaggatttgttgaggttctagccgctcagcagagtccagagaatcccaattggttccaggtgatgaaatcaagcttagatttattctttcttgattagaagtgtgctgaaacctaccattttgtttagattattgtttctgtgaacattaatactgtcgatttcagtatgcattttctttaagtttggcttcatattctgcagggcacagctgatgcagttagacagtacttgtggttatttgaagagcagaatgtgttggagtatcttgttcttgctggcgatcatttgtatcgaatggattacgagagatttattcaagcacacagggagactgatgcagatatcactgtagctgctctaccaatggatgaaaagcgtgcaactgcatttggtttgatgaagattgatgaagagggacgcataattgagtttgcagagaaaccgaaaggagagcaattgaaagcgatgaaggtgaatccaaactcaaatgctttacagatgcgtttgcgatgttgagttggttattatagtcggtgattattcgagtttgtgggttatcatatcctatgtttgaggtgcatattattttagttttaatagaaaatagtaaacaatattgactacacttaattgtcggttataaatgattttagacatcattattatttttagatccccaaacatggagtgaactataataattatttggatgattagttcaaagcttaacaatcaccttttatttcatacaggttgatactactattttgggtcttgacgatgagagagcgaaagagatgccgtacatagctagcatgggtatatacgttatcagcaaggatgtgatgctcaatcttcttagagataaatttcccggagcaaatgattttgggagtgaagttattcctggtgcaacttccattggaatgagggtaagtaccaaataatggtggccttaactatctagttgtctgatgcaaactttactcattacgtgtatctttataccatgattaaaaaatgagaagtaaataaaagaaaaatggaaaagtactagatgaatgagagtgctgatcttgaactcgtgtcactttttgattattgttggtttgctggagaaaaatggcttcagagcttttattggagctacgatctctacgataaagatgttgctttaactttataggtgcaagcttacttgtatgatggttactgggaagatattggtaccattgaagctttttacaatgcaaatctggggatcacaaagaagccagtaccagattttaggtaattttgttaagtttgattcggtttcctcactttttttcctataaaattgtcaaattgtttgtttatttgatacacgtaacaaccaattgcagcttctatgatcgttcgtctccgatctacacccaacctcgttatttgccgccatcaaaaatgcttgatgccgatatcacggacagtgttattggtgagggatgtgtcattaaggtaacttctttactagtaaagtagatctttgctttgctaagatggtagtattgtactattgttacattattgttactttgtctccactgtattatatcttcccctttccttcagtttggaataaacgccctttttattctctcctcagaactgcaagattcaccactctgtcgttggcattcgaacttgcatatccgaaggtgcaatcatagaagatacgttactgatgggtgcagattattatgaggttcgataaccttctttatcacgagtttgagatagattctcaaaattgttcgattatgtagttaaacaacatgaacatgttggaaatcacttttaatcagtaaagttaccatttgaaactcacctaaagcaaaaattgctttaagtgcattctaggctacactacgcctatcctttaatcagatgtgtaatgatgggtgcatccaagtatctctatcttgaaatggagagcatattggatcctgtttgttttgcactcagtttgtcgagacgaaaaattgatgggttgtgagcataaactggtttgtttttcttgtggtgttcttcagacggatgcggaccgtaggttattggcggcaaaaggaagtgtaccaattggaattggtagaaactctcacatcaagagagctatcattgacaaaaatgctcgcatcggtgaaaatgttaaggtttgtcttgtcaacttgtgctttcctttatatttcataaaaatatttactttgtaatgctaacttcaagtagtctttattatatcttatcatgctttttcggggtggggtctcatttgggtgttggttcctatcatctgtttaattatcaatcaaatccattgcttcatcgggaaagaacaaacacaagtagacaagaacctgagttcattagagcttcctttttctcttaaagtaatttttgacggtggaaataaatctattttaaatcagctccacatggccactcacttgccttctgcccttttcatttcttgtttcgattttttgggagtatcggattaaagtgtcaatttttaataacgtgtctaataggttcgttacttgctacgagttttgtttaaatacttgcaacaccaaacatttcacccggttaaattatatatacaccataaccggacgagtgtttaatagatttaaagacattaacatatgaaatcttttaattacaaggattatatattgtctagaaagaaagtaagtaattgccaatttgagatttttttagacatttattacacctctcaatttctttttttattatgtattctactaatgtttttcaaaaacctaagccaagttttaaaatagtaaccatctacttttttatttttaaaaattgtacttattttctctcaattcttatcatagatttcatttttatcaagtaaaagagttcaattcttatcccaaattaaaaaacaagtatacattcttaaaaacaattttttttagtttttaaaattcgacttagtttataaaaaaaaaaaagaaaaccagttgaagtaaaaaacaagaaagagaagtcaagtaggtaaagtaaaataaatgaacacgatcctgtaatcatacgaattgttttgttgggcgtgataatgtaatcatataaactgttttgttgcgtgtgataatgtatgttgtgagtcggcggttgataataataataataatactaatactaatctcacactaacagatagtgaatggagacaacgtgcaagaagcagcaagagagacagatggttacttcataaagagtgggattgtgacagttatcaaggatgctttgattcccagcggcactatcatctaaacaccagaacacatcacacttcactcaactggtgccactctctctctctctctctctctctctctctcnnnnnntctctctctctctctctctctctctctctctctctctctcaattttgtgtctgccttgttcaatcccacccattatgtccacatgcattcccttttggtagtaaatggcaaagcttaggaagagaatccctcactccacagtccacactatacccactgttacttttaacttatgatgatgattatgtaaatgtctccccccctcccctcccctcccctcccctttcctctgctttgaatagtttcatttccttttcttttaacccataaataaattcaatttcctggggcaaaagtggtattatgaatataagtcccattttccat

SEQ ID NO: 92 - DNA, 1581 bp, Cucumis melo
  source 1..1581: mol_type = genomic DNA
atggcgtcttctatggctgcagttggagttctcagactcccgacgtcgtcgtcgtcgtcgtcttcatcgtccaatggtggttccaaccgtgctaggagaactagccttcgaagcctctcatttggtgcgtctcaaatttctggtgataagattgactttagaggttttggtttgggatcacgacgtgtttctggtggtagagtggctccctcgatcgtttctcctaaagcggtttctgattccaagaactctcagacttgcctcgatcccgatgctagccggagtgtgcttgggattattctgggcggtggtgctggaactcggctctatccgcttactaagaaacgagcgaagcctgctgttcctcttggagcgaactataggttgattgatattcctgttagcaactgtctcaacagcaatatttccaagatttatgttctcacgcaattcaattctgcttctcttaatcgtcatctttcccgagcttatgcgagtaatatgggtggttacaagaatgaaggatttgttgaggttctagccgctcagcagagtccagagaatcccaattggttccagggcacagctgatgcagttagacagtacttgtggttatttgaagagcagaatgtgttggagtatcttgttcttgctggcgatcatttgtatcgaatggattacgagagatttattcaagcacacagggagactgatgcagatatcactgtagctgctctaccaatggatgaaaagcgtgcaactgcatttggtttgatgaagattgatgaagagggacgcataattgagtttgcagagaaaccgaaaggagagcaattgaaagcgatgaaggttgatactactattttgggtcttgacgatgagagagcgaaagagatgccgtacatagctagcatgggtatatacgttatcagcaaggatgtgatgctcaatcttcttagagataaatttcccggagcaaatgattttgggagtgaagttattcctggtgcaacttccattggaatgagggtgcaagcttacttgtatgatggttactgggaagatattggtaccattgaagctttttacaatgcaaatctggggatcacaaagaagccagtaccagattttagcttctatgatcgttcgtctccgatctacacccaacctcgttatttgccgccatcaaaaatgcttgatgccgatatcacggacagtgttattggtgagggatgtgtcattaagaactgcaagattcaccactctgtcgttggcattcgaacttgcatatccgaaggtgcaatcatagaagatacgttactgatgggtgcagattattatgagacggatgcggaccgtaggttattggcggcaaaaggaagtgtaccaattggaattggtagaaactctcacatcaagagagctatcattgacaaaaatgctcgcatcggtgaaaatgttaagatagtgaatggagacaacgtgcaagaagcagcaagagagacagatggttacttcataaagagtgggattgtgacagttatcaaggatgctttgattcccagcggcactatcatctaa

SEQ ID NO: 93 - protein, 526 aa, Cucumis melo
MASSMAAVGVLRLPTSSSSSSSSSNGGSNRARRTSLRSLSFGASQISGDKIDFRGFGLGSRRVSGGRVAPSIVSPKAVSDSKNSQTCLDPDASRSVLGIILGGGAGTRLYPLTKKRAKPAVPLGANYRLIDIPVSNCLNSNISKIYVLTQFNSASLNRHLSRAYASNMGGYKNEGFVEVLAAQQSPENPNWFQGTADAVRQYLWLFEEQNVLEYLVLAGDHLYRMDYERFIQAHRETDADITVAALPMDEKRATAFGLMKIDEEGRIIEFAEKPKGEQLKAMKVDTTILGLDDERAKEMPYIASMGIYVISKDVMLNLLRDKFPGANDFGSEVIPGATSIGMRVQAYLYDGYWEDIGTIEAFYNANLGITKKPVPDFSFYDRSSPIYTQPRYLPPSKMLDADITDSVIGEGCVIKNCKIHHSVVGIRTCISEGAIIEDTLLMGADYYETDADRRLLAAKGSVPIGIGRNSHIKRAIIDKNARIGENVKIVNGDNVQEAARETDGYFIKSGIVTVIKDALIPSGTII

SEQ ID NO: 94 - DNA, 4877 bp, Cucumis melo
  source 1..4877: mol_type = genomic DNA
aaacaattgaaactttgctaaatttagcttgaagggtattatttaggtgcatgaagaacacccaaacaatacattgattcgaataaaagaagaaaaagtatatatttccaaaagaattggagatatttatatatggaattatgtggcttccataaattatatacactacgaggaggaagagagttgagcatgtgaatacagttgaagatgaagtggacaattcggcaatagacgaaacgaagacaaccttttagaatattctgtcgattcgtattttgattgatatcaaacgagagggcttaaatctttaatggggttttccattgatcgccattcacataattggtggttgaaattcattcagcaaggataacttttccccaatccttacttccaactctcatgcactccatgtgaaaacactcctttctcaattacgtaactttctaatctacatttctcacaatggccgtcgcttccgattgccggattgccctatcggcggccgcacccgccacttcactggtggtccggaggaagttgaggtctgtggggtttggcaatggggaattcatggggaagaaacttacggaaccgaaacgagtacctgctgtttctgttgctgttaacggtgttccgcgacgtgtttgcatgtctctcactactaatgtagccattggcgatgccgcggtaatgttcttactctgcagattttttttttttttttttggtttcttgtgttgtgaagcttgnnnnnnnnnnnnnnnnnnngtttcttgtgttgtgaagcttgaaatgccttaggtttatgaaactacaccttgtttgttgattcacattgatttctgagagtttgtttgttaacatgttggaccgttggtgatgatctgtctctgtttttcgatagaatttcctctgtaggacgttactctccaatatatggtttgaacattagagaagactggtaattaggttgccaactactatggtatttactcatttgaatttttctctattaccctacgccttctggttgttccagttgaatctatgaacttggtgcaatttggactcagtctgatatgtatatatacgaacaaacctgagatataccattgggaatggaaacaagggactgaaaaaccgttagaccttcataaatctttgtgaggcctattcagaagtattgatctgaggcaatgatgaatgtgttgttgcttgtgactttgtttagttgagagaaatagatgtggagaaaagggaccctcggactgtggtggcgatcattctggggggaggagccggcactcgtctattccccctcaccaagcggagggccaaacctgctgtaagtttcctctactgaactcaatttttcttatagcgatagtgatcatctatggttatttcagtttagatgattggaatttactgacattttatttgtgtgtgtgtgtgtgcgcgcgcgcgcgcgcatgtggaattttgctacgatggctgctgtgatttcttatgatttcccattacaggttccaataggaggagcatataggctgatcgacgtgccaatgagcaattgtatcaatagtggaattaataaagtctatattctcactcagttcaattcagcatcattaaataggcatcttgctcgagcttacaacttcagcagtggagtcacctttggagatggctatgttgaggtacgtttcattcacataaaacatgtcccttaagaattaagagaatcatatacccacaatcatattccccaaaccaaatatgacttatagagctttcagttatatcttaggttcttgctgccactcaaacgccaggggaggctggtaaaaattggtttcaaggtacagcagatgccgtgcgacaattccactggctttttgaggtaccaaaaaagaaaggaaaattcttatctaatagcacagtgagcaaattattgtattgagaaggatataaagaaatgattgttcatgacattttggctgttccatatacaatattcaacataaaaacaagacaaagtgacaaaagaaaatatcgaaatgttaaacagtgtgataagcttttcctggtgcaggatgctagaagcaagaatattgaagatgtgttgattctgtctggagatcatttgtacagaatggactatatggattttgttcaggtaaggctgaagatgacataaagaaccaatctttatttgttattacgtttagttctgatacatcttctttacattggcagaatcataggcaaagtggtgcagatatcactctttcctgtctgccaatggacgataggtgagtgacatcttgatgttttcttctcgacaattagcttaaaagttattgtattaaactactgatcgaccaaaagcttaagcgcgtggtttatggtaaatttcattacccctcacttgtgaaactgaaatttgtaaaaaacccaacaaatgaaaatcaatattaatggggaggaaatgacaatataagagattgaatgtgagactttcttctttgatagcatgtaaaatcattaattgaccaaaaagcttaaacccatagattatgttaaatataattatattagttttaacacgttgatcttgatcatgcaatgagttcaaacctttttatattcgtaggtcttaaggttcaaactatttcagaaatatgtaagaaaggaggagcatttttatttcaaatttacgtttcttttaattgagtatctaatactcctgcaatttatttacaaaacacattaccttttattgtagccgagcctcagactttggtctaatgaagatagacgaaaaaggaaggataatttcattcagtgaaaagcctaaaggacaggacctgaaggccatggtaacattcaaaattgctttcactagaaaattaaagcggtggaggaactttaaatgatcttgttgatgagcctaaactgtatcatctatattcaaattgaaggcagtagatacaaccattttaggactctcgaaagaagaggctcagaagaagccatacattgcttccatgggagtttatgtctttaagaaggacatacttttgaatcttttaaggtattatgcacctaatgctatttatcaatctagtaattatatacaaactcggaggactttgtcgaacacaaaatattagacgttgcttgaatttgttcaagtcagatggcgctttccaactgctaatgactttggatcagagattatccctgcctcggccaaagaattctttataaaggtttgtaatttgtatgtaacctttctgtcgaactgttatatcatctttttcaactttctacttgcaatttagacacaaactaatgctaacctcaacaaaattttcacaggcttatctcttcaatgattactgggaagatattgggacaattagatccttttttgaagcgaaccttgccctgacagagcaagtaagacaactgaacatttatttacaatttcttaccctctttcaccctttagtagttcctcaaattactagaattgtaatttgatttttttttttatatcgtgctaatcttgctgttcaatctcatgtttgcttatacctttcagccaccaaggtttagtttttacgatgctgcaaaaccaatgtttacttctagaagaaacttgccgccttcaaagattgaccagagcaaggtcagtggcattttgcactcatctaaatctctattataaaatcataaacttttataaaaccttcattcttgtttaacgaaatatgctgtagactgccttaaaataacaggcatttggcatgcataaacctgctgagcattcttctggttacctgccaaattttctttgcttgttatgttgagaaaatatctgggttatttgcagatcgtagattctatcatctcacatggaagcttcttaaacagttgctttatagagcacagtgtcatcggtatccggtcgagaatcaactcaaacgttcacttgaaagtatgtatttcatggaacttttgcatgatacggtatagtgataagaaaaatatatatgtatatgaaaatttgtcaaaacactgtttattattattttaaaagaagataccttttgatgcaatataaagatatcacaccataaacaatgctttgtgatgagtcgcaaaatgcaggacactgtgatgcttggagcagattactatgaaactgaagatgaagttgcatctttgttagctgaaggaagagtaccaataggaataggggaaaatacaaaaataaagtatgatgaaccccatcacacccaaaaaaaggatcttcatatgttctatagcctgcttttctaattcttcattgttataaatattatgtgcagggactgcattatcgacaaaaatgctagaatcggaaagaatgtggttattgccaattcggaggtaccgttaccataagagtaaaagaaagaatcttagttatattcgtgaaacgaatagttttgaaatgactttggaataaataagttatgtttcattggaatcgtattgtagggtgtacaagaggcggatagatcttcagaaggcttctacatacgttctggcgttaccatcatcttaaagaatgcagtaattaaagatggtttggttatataataaaatttgttcatccctcattgatgaacatgccaaccgttcattgatttgtattatttctttatttattttaaattttctgtatcgcataaaataagaaaagcagaggatgtaagctaacagcgaaatatatctatgaatgtgattgttcaacacgactatgccaagtagactgtttttatttgggcaacttgctttgaagaaaaaaaatatataccatt

SEQ ID NO: 95 - DNA, 1614 bp, Cucumis melo
  source 1..1614: mol_type = genomic DNA
atggccgtcgcttccgattgccggattgccctatcggcggccgcacccgccacttcactggtggtccggaggaagttgaggtctgtggggtttggcaatggggaattcatggggaagaaacttacggaaccgaaacgagtacctgctgtttctgttgctgttaacggtgttccgcgacgtgtttgcatgtctctcactactaatgtagccattggcgatgccgcgttgagagaaatagatgtggagaaaagggaccctcggactgtggtggcgatcattctggggggaggagccggcactcgtctattccccctcaccaagcggagggccaaacctgctgttccaataggaggagcatataggctgatcgacgtgccaatgagcaattgtatcaatagtggaattaataaagtctatattctcactcagttcaattcagcatcattaaataggcatcttgctcgagcttacaacttcagcagtggagtcacctttggagatggctatgttgaggttcttgctgccactcaaacgccaggggaggctggtaaaaattggtttcaaggtacagcagatgccgtgcgacaattccactggctttttgaggatgctagaagcaagaatattgaagatgtgttgattctgtctggagatcatttgtacagaatggactatatggattttgttcagaatcataggcaaagtggtgcagatatcactctttcctgtctgccaatggacgatagccgagcctcagactttggtctaatgaagatagacgaaaaaggaaggataatttcattcagtgaaaagcctaaaggacaggacctgaaggccatggcagtagatacaaccattttaggactctcgaaagaagaggctcagaagaagccatacattgcttccatgggagtttatgtctttaagaaggacatacttttgaatcttttaagatggcgctttccaactgctaatgactttggatcagagattatccctgcctcggccaaagaattctttataaaggcttatctcttcaatgattactgggaagatattgggacaattagatccttttttgaagcgaaccttgccctgacagagcaaccaccaaggtttagtttttacgatgctgcaaaaccaatgtttacttctagaagaaacttgccgccttcaaagattgaccagagcaagatcgtagattctatcatctcacatggaagcttcttaaacagttgctttatagagcacagtgtcatcggtatccggtcgagaatcaactcaaacgttcacttgaaaatatcacaccataaacaatgctttgtgatgagtcgcaaaatgcaggacactgtgatgcttggagcagattactatgaaactgaagatgaagttgcatctttgttagctgaaggaagagtaccaataggaataggggaaaatacaaaaataaaggactgcattatcgacaaaaatgctagaatcggaaagaatgtggttattgccaattcggagggtgtacaagaggcggatagatcttcagaaggcttctacatacgttctggcgttaccatcatcttaaagaatgcagtaattaaagatggtttggttatataa

SEQ ID NO: 96 - protein, 537 aa, Cucumis melo
MAVASDCRIALSAAAPATSLVVRRKLRSVGFGNGEFMGKKLTEPKRVPAVSVAVNGVPRRVCMSLTTNVAIGDAALREIDVEKRDPRTVVAIILGGGAGTRLFPLTKRRAKPAVPIGGAYRLIDVPMSNCINSGINKVYILTQFNSASLNRHLARAYNFSSGVTFGDGYVEVLAATQTPGEAGKNWFQGTADAVRQFHWLFEDARSKNIEDVLILSGDHLYRMDYMDFVQNHRQSGADITLSCLPMDDSRASDFGLMKIDEKGRIISFSEKPKGQDLKAMAVDTTILGLSKEEAQKKPYIASMGVYVFKKDILLNLLRWRFPTANDFGSEIIPASAKEFFIKAYLFNDYWEDIGTIRSFFEANLALTEQPPRFSFYDAAKPMFTSRRNLPPSKIDQSKIVDSIISHGSFLNSCFIEHSVIGIRSRINSNVHLKISHHKQCFVMSRKMQDTVMLGADYYETEDEVASLLAEGRVPIGIGENTKIKDCIIDKNARIGKNVVIANSEGVQEADRSSEGFYIRSGVTIILKNAVIKDGLVI

SEQ ID NO: 97 - DNA, 4779 bp, Cucumis melo
  source 1..4779: mol_type = genomic DNA
gagaagctgaaagagtcggtcccatcccgtttgcgaaacactcttattgccgccgtgttcaactcccaaagcggccctctcatgagaaaaaaatcaagatcgccagacttttccatggctccccaaacccaaataatttaatcacgctaatttccatacctttcacattcatcttctccttcactcgatctttcatctttctcccattcccgcctttcttcttccctcctacactcttcattctacatttctctcgctccttcttcttaggtttcccactgatctaccccgtttttgggtcacttcatttcaccaatggattcctttggtgtcgccttgaaccccaatacgatgccatttagaatcagtagtcagtgtgttaagaatcaatgctttgggttttggggagacagtagtcttggaagaaacggaaggtggaaacagatacagaggaatgcctcctcgcggaacaattctgattcttcttcttctagcagggctaggaagcttgcacccggagttgcttattctgttctcatgtcagaagttaatgaggaaacaacggtaaatgggttttgtactttgttgttcaaaagtatatgaacttttacatgaattttgacttcaaattttctctctcagactttacaggcccctatatttgaagctccaagagctgatccgaaaaaggttgcttcgattatactgggtggaggtgctggaactcgtttgtttcctcttaccagtcagcgagccaaaccagcggtaaccaactctaccaaattgctaagagaaggataattcgatagtagataacagaagctattgatctaatctaggagaaagagttagtgcacgaatttaagtaaataatatttgcttgcaggttcctattggaggttgttataggttgatcgacatacccatgagtaactgcatcaatagtggcatcgaaaagatattcgtcctaacgcagtttaactcattttctctcaatcgtcaccttgctcgtatttataattttggtaatggagtcaattttggcgatggattcgtggaggtatgatgaatgttctttccattgttgcaatctgacccagcttagtaattgtgttaatcagacatacggcaagttctaggaaacaggaagctttcttatactcaatttctggattcatgtttgagaaacaggttctagctgccactcagacttcgggtgaaactggaaaaaaatggttccaaggaactgctgatgctgtcaggcaatttatatggttatttgaggtgggtgcataaaatctactgagtatgattagaatcttcagtgaattttttaattgtaatgaacatttgtccattgggtctgtgcaggatgctaagaccaaaaatgttgagcacactctaattttgtctggcgatcacctttaccggatggactacatggattttgtgcaggtataatgttaaactgactagtttgtaattgtatttgtagtgttttgcatatttgtttgtgattcaatgagactttctgatggctttgtacttgcagagacatattgacacaaatgcagatattactgtttcatgcatacccatggatgataggtaggcatccattttaatccttattttcatagataaagtgcatggaaaccataatcttattctttatttccttaaaaatttagattttgattaatgttctgttattatgcagccgtgcatcagattacggtctcatgaaaatagatgatacaggacgtattatacatttttcagagaaaccaaagggatctgacctggaagaaatggttggtagcattgatgcaattagttattttcttgaacttcttttgttttctctcttttttatttgtttggttgtgttattagcgtaaatgtatttattggctcctgttccttatagtgccctgtatcgttgtctgattttgttttgtatccatcatatattgtactgcagcaagttgacactgcagttctaggactttcagatgaagatgctagaaaaaatccttatattgcttcaatgggagtctacatattcagaacagatcttctattaaagcttttgacatggagttatcctgcgtgcaatgactttggctcagaaattattcctgcggctgttaaagactacaaagttcaagtgagatccgttttgagaattttagtctatcattttcctttatatgcttgagttcagaaaaggaagactttcgacaataccacaactaagtttcccaatgcatccatattgttgtaggcatatctattcaatgactactgggaggacattggaacagtaaaatcattttttgatgccaacttagcactaacggagcaggtatgtatcccatcatacaagcaattgtgtctcaggccgtcgatggtctcctttctttccgttttatgggatcaagaaacatcttacaccaatattttcagctttcatgcttatttattcatttagttagatatctacatagatgataagctcttatattacttacttctgctcatattctcctgaagtactgataagataccacccttaatccatatcaagtaccttaacagcactgtgaaaatttcaaccaaacccctcccactaatcagaaacaatagatgatcaagtgtaatcaagcaaggtctttctccaaacactattatttacactagctgatggtttgttgcgtctttcatgcagcccccaaaatttgagttctatgatcccaagactccattttatacctcacctagattcttgcccccttctaaagttgaaaaatgcagggtaagacatcaaacttgagttttctttctgcatcatctgctttgttcttgtgtcttaaatgaatctgtatgcagattgtagatgcaattatctcccacggttgcttcctccgagagtgtactgttgaacactctattgttggcgtgcgctctcgactggagtatggtgtggagcttaaggtgagccataattttcttttggactttcatcctatcccatagggaagtacaagaaggctaaatgcagttgcagaagctggctttgtagatgctgcaatttttgtccacagattaaactctttttatttgtgttagagagtccgtcgtgctgattcctaatagtagatggatcaatccaaattgaatacgtaatagatgtttgttactagctgttgccgcatctcttgaaaaaagctaaaatcatatttgaatttgttggttttgcattataggataccatgatgatgggtgcggactactaccagacggaatctgaaattgcatctctccttgctgaaggaaagatcccaattggcattggagagaacaccaaaatcaggtaatattcagctaccaataaagagtaaaagctatgtgcaatcttttattagtttaatataaccgtcactttatcatacgactcattaacctacaaattatttgtttgggcgtttggtcttgtttgtgatggtctgtaacttctctgcaggaattgcataattgacaagaatgccaagatcggaagaaatgtggttattgcaaatactgatgtaagtataatttttgtacttgcaagggatgtttggccaatagttttaaattggttattgcaaaaactttttaaatacattgagtttacaaggttcaaactgttctatttacgatgattcgtatgtgcaaagaaataaacagtaaatacttgggtttggttcctgggtttataacctgggtggatttcaattgccggttcatatattgagttcacgtaattcgtaggattaacagttcgggatgagaaaagccacatgtttttcaaacatcaatttgactttttaagcacaagttacacaagtttagcccaaaatttgaccaccttctaagaatttgcaacctcaatttaaaccaaataagcgcataatgttatttgattatagtgcctatgcttaagaagtgtacttattaacttttcaggacgttcaagaagcagatagaccagaagaaggtttttatataaggtccggtatcacagtcacgttgaagaatgcaacaataaaagatggaaccattatttaaaatccaaaggcagcctcccattcaggtcaagagatagctaagagttgaaaggaagcaattgctagaggctttcaacctacaatgctcggcaacgagtaatagcgtataacttcccttaatgtaaaatactagtttcttttcatatatgtaaaaagtaggaaggcaacacatttccatcctgagtaataacaactccttatatcataagagaacaattttcaaagccatttgacattagcttcttggatgagcaaatctcatctctgtgaaagagttactctcttaaaactcacaagttatattaagttgttaatatttgcttggcatttctattttcagctctatttttacttaatttccaggactattgttgcccccccttgaggcctcccactgtgcctttttcatctcctttgctctcttccacttctcaatgtttctgagtttggcatacatccttctgatgtgaatcagccttctagactcctgggtgatcaaatttgcagtcgaggggctctcgcttagtaccacttcatacccatctcgaaaagaatccatcccttcaactagaagttgccagaacattccaccgatggctgcaccaccccctcgagcagacgaatacacagcagagtaaacagcattgaaaagctgatccctttgatcagc

SEQ ID NO: 98 - DNA, 1191 bp, Cucumis melo
  source 1..1191: mol_type = genomic DNA
atgagtaactgcatcaatagtggcatcgaaaagatattcgtcctaacgcagtttaactcattttctctcaatcgtcaccttgctcgtatttataattttggtaatggagtcaattttggcgatggattcgtggaggttctagctgccactcagacttcgggtgaaactggaaaaaaatggttccaaggaactgctgatgctgtcaggcaatttatatggttatttgaggatgctaagaccaaaaatgttgagcacactctaattttgtctggcgatcacctttaccggatggactacatggattttgtgcagagacatattgacacaaatgcagatattactgtttcatgcatacccatggatgatagccgtgcatcagattacggtctcatgaaaatagatgatacaggacgtattatacatttttcagagaaaccaaagggatctgacctggaagaaatgcaagttgacactgcagttctaggactttcagatgaagatgctagaaaaaatccttatattgcttcaatgggagtctacatattcagaacagatcttctattaaagcttttgacatggagttatcctgcgtgcaatgactttggctcagaaattattcctgcggctgttaaagactacaaagttcaagcatatctattcaatgactactgggaggacattggaacagtaaaatcattttttgatgccaacttagcactaacggagcagcccccaaaatttgagttctatgatcccaagactccattttatacctcacctagattcttgcccccttctaaagttgaaaaatgcaggattgtagatgcaattatctcccacggttgcttcctccgagagtgtactgttgaacactctattgttggcgtgcgctctcgactggagtatggtgtggagcttaaggataccatgatgatgggtgcggactactaccagacggaatctgaaattgcatctctccttgctgaaggaaagatcccaattggcattggagagaacaccaaaatcaggaattgcataattgacaagaatgccaagatcggaagaaatgtggttattgcaaatactgatgacgttcaagaagcagatagaccagaagaaggtttttatataaggtccggtatcacagtcacgttgaagaatgcaacaataaaagatggaaccattatttaa

SEQ ID NO: 99 - protein, 396 aa, Cucumis melo
MSNCINSGIEKIFVLTQFNSFSLNRHLARIYNFGNGVNFGDGFVEVLAATQTSGETGKKWFQGTADAVRQFIWLFEDAKTKNVEHTLILSGDHLYRMDYMDFVQRHIDTNADITVSCIPMDDSRASDYGLMKIDDTGRIIHFSEKPKGSDLEEMQVDTAVLGLSDEDARKNPYIASMGVYIFRTDLLLKLLTWSYPACNDFGSEIIPAAVKDYKVQAYLFNDYWEDIGTVKSFFDANLALTEQPPKFEFYDPKTPFYTSPRFLPPSKVEKCRIVDAIISHGCFLRECTVEHSIVGVRSRLEYGVELKDTMMMGADYYQTESEIASLLAEGKIPIGIGENTKIRNCIIDKNAKIGRNVVIANTDDVQEADRPEEGFYIRSGITVTLKNATIKDGTII

SEQ ID NO: 100 - DNA, 5160 bp, Cucumis melo
  source 1..5160: mol_type = genomic DNA
ctccaatccaagtatagccttgtttgaatgcggcgtacaaatagcggatgatcgttgcaaattgagttttgtagaaaaataatatttttaaaaaaaaattatgaagaagaagatgatgaagaacaaagaacaagcatttgctgacatgaatgaatgaacctttcctactttcttgttttttcctataaaaatcaatcctcgtgaatgaaaacgccttacattcacatgcccataacccaccgccttcttcatcactcacatttttcactcccttttcccctttctctcttcttctgcttttgatccgtttcctctttgtaagtttttcacttattctcctttccctttctagattttcttgttttcatcactttattatctgggtctctcttattttattgtatttcaaaattttcaggtgatttcttccattttttagctaccccttttacctttttctttcctttgtgtgatctgtactctatttggttgcttggattgtggcttttgctttttctattgggatttgagctgggggagatgctatttgattgtaattttgtgacattttctgatgcattgtttttttttttttttgtagagagttaactgatttttttggatttggtattgttagtgagatatggttgcgatggattcctgctttgtctctttgaaatccaatacccaattgatgaagggtaattggggaggtttggatcgttgtgagaatggattttatggtgagaaagttagaggaggttttaatgaaaatgtttggattaagagtttgaaatatgagaagaaagctttgaagcttaccccaaatgttgcttatgctgtggctacgcctaatgtttcaaagcagcctatggtgagtttactgcatttatggatggccatatggccatgtttgattttatatctttggaagttttgatgaaaaccccatcatgatttgtcttcatttttcatttctttgaattggatttgattttgtagaccattcaagttccaacagttcctaaagtaaaagcgaatcccaaaaatgttgcttcaatcatattgggagggggtgctgggactcacctgtttccgcttactaaaagatcagcaacgcccgctgtaaggaactagacttgtgtttgtctgtgtgtgtatatatttgattgtgcaacttttcaagggtcaacggcttagtttagatcattttcgaatcagttctctgttttttacaccaaagaaaatgtacttttgttctcgctgatgttaatgtatttcttagaatatatgaatctgtttttttttttttttactttccaaatcatgtgttgaaggcaggttttcctaatatctttgccgttttttatggtgtgatcatatagttcaggttatttgtcatcaggtcttgtatgtttttattagactaagcagttggataatttatacaatttaactatcaaatatttgcttgcattacatgggtaggttccagttggaggatgctataggcttatagatattccaatgagcaactgcatcaacagtgggatcaacaaaatatttgtgcttacccagttcaactctgcttctttgaatcgtcatatctcgcggacatactttggaaatggtgtcacttttggggaaggatttgtggaggtatggtatggcaatgtttaggaagctatcgacaaaagcttcaattgataatcgaagaacttaatgataaaaaacatggaatcgaaaagaaacttagagatagagttaattgtttttcctagcttgactgttttattttggaaactgagagtgtacagttgtaattattttcagtagttagttatttcagatctctagattataatatgaccttgagctcactcttgataaacaggttcttgcagccacacaaacatctggggaatccggtatgtactggttccaaggaactgcagatgctgtgagacaatttatttgggtatttgaggtataacacaattttattttctttttgttctgtttgaaacttcaccgtgttgatattataaagttgattttcgtattatttcttgtaggatgccaagaacagaaatgttgagaacattctaattttggcaggggatcacatgtacagaatggactatatggactttgttcaggtaatgaatttctttttgaaaccccattctgataaagataatagttcgtattctatatttcaaaatctaattgtatgctatcattggacacagaatcacattgatcgcaatgctgatatttcaatctcgtgtgcagctgtggatgacaggtcttatcaatgttccttttaccttataattcttcgtttaaagttgttttgcttgtaggtaatagtaaaagatttgataagtgcaattgtttgtctaagttgacttcagaaccttcttttgtatgcttaaagaacaacgaaattgaaactcttctagatcatacgaatttatctatttgttctttagaattttagagccaaagtctcatatctttttaatccactctgcagccgcgcatcagactacggattggtgaaattagatagtagaggtcgaattatccagttttctgaaaagccaaagggtgccaatttgaatgcaatggtaaaaacttcagaaaacatagaatggttattttttccactttgtgaatgaactgttggcttaacacatgaatgccattaaagccatcattcttggctgaaattggttatgtctactgaaccttctcttcaaccattgtctctcaaactacattcctaatttaagcatcaaaattttatcttgtaatttgagtcttgttcatgtggcataaccatcccaattggttggtattctgcagcgagtagatacaacttcatttggtctgtctcgggaggagtctttgaagtctccttacattgcatcaatgggagtttatgttttcaaaacggatgttttgctaaaccttttgaagtggagatatccttcatctaatgacttcggctctgaaatcattcctgcagctataaaggaccacaatgtccaagtaagatgattttctatcaactcgtaacgcttcattaattaaatttgtcaaagtaataattgtgttgacctactgtgttcttgaatcttggattaatatcttgaactggtaaaattttgtatttctgtgataacataatggttcttgatacaaaatgctgcataatttaagttcacctacaatcaagttcaagtgctaaaagcccttttttagctgtaactattaaaagataactcctattttgacttccatttctcttctatcatagaacctatctcaaaccctcatatatcaacttcatttgagacattgtttccacattgatgtattatgtgtgagtttaacttttatttctttcttcttccttgttttcttattctaaaggcatttatgtttagagattattgggaggacattggaacaataaagactttctatgatgcaaacttggccctcacggaagaggtatgttacatttcctggactcgaatttttttcgttctggtttttttacggttgtatgatcagtccgtgtagtttattacttgactttgcaatctttatcttaagaacctgaactgtggaattaactgaaggtcatagtcctttgtttcaagcaatgcattctgtgctgaaaggattcattttctagaacttaccggacattgccaattgcttcatttgattatcaagttttcattttctcgcagtttcctaagtttgaattttatgaccccaagacacctttctatacgtctcctcgattcttaccgccgaccaagatcgacaggtgtcaggtatatgagtaaaaccaaactgattctaatctcgtttttaggactctgctcttaagcttcatttgagttctgcagattgtcgatgcaataatctcacatggatgctttttgagagaatgtagtatccaacattcgatagtcggtgaacggtcaagattagactatggtgttgaacttaaggtgaaagcgaagaactcatagtttcagacaaagccacaggttgtaggacaaacacttggatttgtaaccgtggttgctatcttctatttttgcaggatacgataatgatgggtgcagacaattaccaaaccgaacttgaaattacaggtctgctagcagagggaaaagtccctgtagggattggaccaaacacgaaaatcaggtaagtcgactcgaaccttgagagaaacttgttaaacatagatctgcgcaagaatcttccaatccaacgatattgagttatcattatgggtgtggtttgttgcaggaagtgtataatcgataagaatgcaaaaattgggaaggatgttatcattatgaacaaagatgtaagtatatctcaacccatcctcaacgcattcagtcttgaaaacctcattcttctctttccattaatctgtttctctatcaatttccagggcgtccaagaagcagatcggcctgaacagggattctacattcgctcgggaatcaccattgtaatggaaaaggcaacaatcgaagatggcaccgttatatgaacagaactcaaaatcctcaatcttggtttggaagacatgttagaggaggaaggaaaatggtgtgaagttggctttcatgagactgcctaaactgtggcagctgaagaatcagcattgtagcttgagaaaaaggagaaaggatttgattatggagattgagtttaaagcttcttttcagatctttagagatagcttttaagaggttcgacaaaaaggaagttccacatggagaagagagtctaaatctttagtgtaattctgttcaataagaatggctacacagccagattagtagggttatgtaaataggaatattatgttgatgtatcatccataatcattccttctataaagttgattgatatttggtgcacttccaagtttcaagacttgggagagtgtttaaattataatcaacatcaagatctttgattaatggccatctgaattcatatatttcattttatactgtgtcaaatcaatctggttttatgcttgttgagtaacaaaactaatctatacgttagtaatggtcactttttgtaaatatgggtgtaatagtaaag

SEQ ID NO: 101 - DNA, 1608 bp, Cucumis melo
  source 1..1608: mol_type = genomic DNA
atggttgcgatggattcctgctttgtctctttgaaatccaatacccaattgatgaagggtaattggggaggtttggatcgttgtgagaatggattttatggtgagaaagttagaggaggttttaatgaaaatgtttggattaagagtttgaaatatgagaagaaagctttgaagcttaccccaaatgttgcttatgctgtggctacgcctaatgtttcaaagcagcctatgaccattcaagttccaacagttcctaaagtaaaagcgaatcccaaaaatgttgcttcaatcatattgggagggggtgctgggactcacctgtttccgcttactaaaagatcagcaacgcccgctgttccagttggaggatgctataggcttatagatattccaatgagcaactgcatcaacagtgggatcaacaaaatatttgtgcttacccagttcaactctgcttctttgaatcgtcatatctcgcggacatactttggaaatggtgtcacttttggggaaggatttgtggaggttcttgcagccacacaaacatctggggaatccggtatgtactggttccaaggaactgcagatgctgtgagacaatttatttgggatgccaagaacagaaatgttgagaacattctaattttggcaggggatcacatgtacagaatggactatatggactttgttcagaatcacattgatcgcaatgctgatatttcaatctcgtgtgcagctgtggatgacagagccaaagtctcatatctttttaatccactctgcagccgcgcatcagactacggattggtgaaattagatagtagaggtcgaattatccagttttctgaaaagccaaagggtgccaatttgaatgcaatgcgagtagatacaacttcatttggtctgtctcgggaggagtctttgaagtctccttacattgcatcaatgggagtttatgttttcaaaacggatgttttgctaaaccttttgaagtggagatatccttcatctaatgacttcggctctgaaatcattcctgcagctataaaggaccacaatgtccaagcatttatgtttagagattattgggaggacattggaacaataaagactttctatgatgcaaacttggccctcacggaagagtttcctaagtttgaattttatgaccccaagacacctttctatacgtctcctcgattcttaccgccgaccaagatcgacaggtgtcagattgtcgatgcaataatctcacatggatgctttttgagagaatgtagtatccaacattcgatagtcggtgaacggtcaagattagactatggtgttgaacttaaggatacgataatgatgggtgcagacaattaccaaaccgaacttgaaattacaggtctgctagcagagggaaaagtccctgtagggattggaccaaacacgaaaatcaggaagtgtataatcgataagaatgcaaaaattgggaaggatgttatcattatgaacaaagatggcgtccaagaagcagatcggcctgaacagggattctacattcgctcgggaatcaccattgtaatggaaaaggcaacaatcgaagatggcaccgttatatga

SEQ ID NO: 102 - protein, 535 aa, Cucumis melo
MVAMDSCFVSLKSNTQLMKGNWGGLDRCENGFYGEKVRGGFNENVWIKSLKYEKKALKLTPNVAYAVATPNVSKQPMTIQVPTVPKVKANPKNVASIILGGGAGTHLFPLTKRSATPAVPVGGCYRLIDIPMSNCINSGINKIFVLTQFNSASLNRHISRTYFGNGVTFGEGFVEVLAATQTSGESGMYWFQGTADAVRQFIWDAKNRNVENILILAGDHMYRMDYMDFVQNHIDRNADISISCAAVDDRAKVSYLFNPLCSRASDYGLVKLDSRGRIIQFSEKPKGANLNAMRVDTTSFGLSREESLKSPYIASMGVYVFKTDVLLNLLKWRYPSSNDFGSEIIPAAIKDHNVQAFMFRDYWEDIGTIKTFYDANLALTEEFPKFEFYDPKTPFYTSPRFLPPTKIDRCQIVDAIISHGCFLRECSIQHSIVGERSRLDYGVELKDTIMMGADNYQTELEITGLLAEGKVPVGIGPNTKIRKCIIDKNAKIGKDVIIMNKDGVQEADRPEQGFYIRSGITIVMEKATIEDGTVI

SEQ ID NO: 103 - DNA, 4528 bp, Citrullus lanatus
  source 1..4528: mol_type = genomic DNA
atggcgtcttctatggctgcggttggagttctcagacttccgacgacatcggcgtcgtcgtcttcgtcatcgaacggcggttctaatcgtgcaaggagacacagccttcgaagcctctcattcggtgcgtctcacatttctggtgataagattgactttagagcttctgctttgggatcacgacgtgtttccggtggtagagcggttgcggttccttcgatcgtttctccaaaagcggtttctgattccaagaactctcagacttgcctcgatcccgatgctagccgggtcggtattctcgtttctttgtgccattatctgaatgcgtttaattgattggaattagatagttttgttgcctgttgtgcttgatgcgttcgtggaatttatttcttgtgattttctgttttttagcggccgtgaattgtttttttttggaatggatggtgattgaatggctttctattatctgttctgttgtgtttagagtgtgcttgggattattctgggaggtggtgctggaactcggctctatccgctcactaagaagcgcgcgaagcccgctgttcctcttggagcgaactataggttgattgatattcctgttagcaactgtctcaacagcaatatttcaaagatttatgttctcacgcaattcaattctgcttctcttaatcgtcacctttcccgagcttatgcgagcaacatgggtggttacaagaatgaaggatttgttgaggttctggccgctcagcagagtccggagaatccaaattggttccaggtgatggaatcaagcttagatttattctttcatgattaatttgtaattagaagaatattgtttctgtgaacaatggcatttggaatgcattttctttaaatttgtcgagaagttttatctcataaaagtttggcatcgtatcctgcagggcacagctgatgcagttagacagtacttgtggttatttgaagagcagaatgtgttggagtatctagttcttgctggtgatcatttgtaccgaatggattacgagaggtttattcaagcacatagggagactgatgcagatatcactgtagctgctctaccaatggatgaaaaacgtgcaactgcatttggtttgatgaagattgatgaagagggacgcataattgagtttgcagagaaaccaaaaggagagcaattgaagacgatgaaggtgaatccaaactaaagtactttttatcttaataaactattctctcatttcttgattcataaacggttccatcttttatatttttgggcaaatatcaatttatacccctaaactctggttatgtcaatttaaactttgaactttctttagtgtgtaccctcttcagattttgttcgaccaatatcttatgatacttatgattttagtcaattaaactcataaattttcataagccaatcaatttaggtgtttcaatatgattatctttgaagatcatcctacattgattctcgaacgtgtatagatatcttcaaatgttggttttacaaaatggacgattgtaaaagtttaaattaattaatttatgaaagtttagggctttaattgatacaaacataagtttcacatgaggctttcccaaacgaaacgtaattgagggtgtaaattgatacattgggtttagattgatacattgggtttagattgatacaattattagttcaagatataaattggtattttcctatattcttttaggctggttagtataaagcttaacaatcctgttttatttctttacaggttgatactactattttgggtcttgatgatgcgagagcgaaagagatgcctttcatagctagcatgggtatttacgttatcagcaaggatgtgatgctgagtcttcttagagataaatttcctggagcaaatgattttgggagcgaagttattcctggtgcaacttccattggaatgagggtaagtaccaaataatggtggccttgaccatctagttgtctgatactaactttaagcattacatgtatctttataccatgattgaaaaataaaaagcaaataaaagaaaaagggaaaaagtactagatgaatgagggtgctgatccacttttgtttattcttgttttgctggagaaagatgacttcagagcttgtatgggagctgccatctctaacaataaagatgttgctttaactttataggtgcaagcttacttgtatgatggttactgggaagatattggtaccattgaagctttttacaatgcaaatctggggatcacaaagaagccagtaccagattttaggtaatttagtcaagtttgattcggtttccacatttttaccattaaattatcaagttgtcatttgtttatttgatacatgaaacacccaattacagcttctacgatcgttcgtctccaatctacacccaacctcggtatttgccgccatcgaaaatgcttgatgctgatatcacagacagtgttattggtgagggctgtgtcataaaggtaacttccgtacaagcaaagtagatttttgctttgttaagatggtattgaactattgttacattgtctccactgtatgattccttcccctttccttcagtttggaataacccctttttattctctcagaactgcaagattcaccattctgtcgtgggcatccgaacttgcatatcggaaggtgcaatcatagaagacactttattgatgggtgcagattattatgaggtctgtggctaataataaccttctttatcatgagcttgagatagctttgagctttttgagctttctttcaagtacttttagattctccaatttgtttgactatatagttcagccacgtgaaaatgtttgaaaactacttttaatcggtaaagttaccattcaaaacttacctatggctgaaaatattaggtgcatcctaccaaattgtccaatcataatttaccactttgcaaattcactttaattacttattttgaaatagagagccattccaaatgacttgtagttgcaggaccttgtttgtttttcactcgtttgtagagacgaaaaattaaggggttgtgagcataaactgatttgttttcttgtggcgttcctcagacggacgccgaccgtagattattggcggcaaagggtagtgtaccaattggaattggtagaaactctcacatcaagagagctatcattgacaaaaatgctcgcatcggggaggatgtcaaggtttgtctttgtcaactcgtgcttgcttctatttcatgtttaacgtttactttttaatgcttacttcaagtccttattatatcttatcatgaattctcggggtggggtcccatctgggtgttgatttccattatctgtttgactatcaatcaaatctattgcttccattgggaaagaacaaagacaagtagacaagaaccgagttcattagagcttcctttttctctttaaagtaattttgacggtgggaataaatctgtttcaaatcagcttcacatggccgcacacttgttttctgccattttcatttcttgtttcgatttttgggaagttaataaaaaaagaaaagtactagattaaaatgtccatttttaattgtgtgtctaataaattcactaattatgtgtaaagtagctactaaatatgtgtaaagtaattttgtttttagacttgctacaccaaacacttcagcatgtctgaaatcaaccaaattaaataatatactctccataactacacgataataacatattaaacacagagaaaagtttaattacaaggattatatattgtctagaaagaaagtatagtgtctattgtcgatttgagcatatctcacttgatttactgtaggtccgtttgataaccatttggtttttatttttagtttttggattcatcccacttctaaatttcttgtttattatctacattcttccatcattcgaaaaaatcaagccaagttttgaacactaagactctgtttgacaaccatttggtttttgaaaaattgtctttattgttttctctcaatttcttatcatgattttcacatagtcgaattcttaaccaaaatccaaaaacaaaaactatttttttagttttcaaaatttgacatggtttttaaaaatattggtagaaaatagttaacaaattaaaggaatttgaaggtggatgggtacttgtaaacttcatttttaaaaaccaataatcaagaatcaaatagataccggtggttgaattaaaagaaaaaaaaaaagtggtctattggaagctttttaaagtagtttaactaaatgaacacgatcctgttaatcatatgaattttattgttgggtttcatagttttcttacttgccgcttgataatatacattcatctcagattgtgaatggagacaatgttcaagaagctgcaagagagacagatggttacttcataaagagtggaattgtgacagtaatcaaggatgctttgattcccagtggcaccgtcatctaa

SEQ ID NO: 104 - DNA, 1626 bp, Citrullus lanatus
  source 1..1626: mol_type = genomic DNA
atggcgtcttctatggctgcggttggagttctcagacttccgacgacatcggcgtcgtcgtcttcgtcatcgaacggcggttctaatcgtgcaaggagacacagccttcgaagcctctcattcggtgcgtctcacatttctggtgataagattgactttagagcttctgctttgggatcacgacgtgtttccggtggtagagcggttgcggttccttcgatcgtttctccaaaagcggtttctgattccaagaactctcagacttgcctcgatcccgatgctagccggagtgtgcttgggattattctgggaggtggtgctggaactcggctctatccgctcactaagaagcgcgcgaagcccgctgttcctcttggagcgaactataggttgattgatattcctgttagcaactgtctcaacagcaatatttcaaagatttatgttctcacgcaattcaattctgcttctcttaatcgtcacctttcccgagcttatgcgagcaacatgggtggttacaagaatgaaggatttgttgaggttctggccgctcagcagagtccggagaatccaaattggttccagggcacagctgatgcagttagacagtacttgtggttatttgaagagcagaatgtgttggagtatctagttcttgctggtgatcatttgtaccgaatggattacgagaggtttattcaagcacatagggagactgatgcagatatcactgtagctgctctaccaatggatgaaaaacgtgcaactgcatttggtttgatgaagattgatgaagagggacgcataattgagtttgcagagaaaccaaaaggagagcaattgaagacgatgaaggttgatactactattttgggtcttgatgatgcgagagcgaaagagatgcctttcatagctagcatgggtatttacgttatcagcaaggatgtgatgctgagtcttcttagagataaatttcctggagcaaatgattttgggagcgaagttattcctggtgcaacttccattggaatgagggtgcaagcttacttgtatgatggttactgggaagatattggtaccattgaagctttttacaatgcaaatctggggatcacaaagaagccagtaccagattttagcttctacgatcgttcgtctccaatctacacccaacctcggtatttgccgccatcgaaaatgcttgatgctgatatcacagacagtgttattggtgagggctgtgtcataaagaactgcaagattcaccattctgtcgtgggcatccgaacttgcatatcggaaggtgcaatcatagaagacactttattgatgggtgcagattattatgagacggacgccgaccgtagattattggcggcaaagggtagtgtaccaattggaattggtagaaactctcacatcaagagagctatcattgacaaaaatgctcgcatcggggaggatgtcaagttttcttacttgccgcttgataatatacattcatctcagattgtgaatggagacaatgttcaagaagctgcaagagagacagatggttacttcataaagagtggaattgtgacagtaatcaaggatgctttgattcccagtggcaccgtcatctaa

SEQ ID NO: 105 - protein, 541 aa, Citrullus lanatus
MASSMAAVGVLRLPTTSASSSSSSNGGSNRARRHSLRSLSFGASHISGDKIDFRASALGSRRVSGGRAVAVPSIVSPKAVSDSKNSQTCLDPDASRSVLGIILGGGAGTRLYPLTKKRAKPAVPLGANYRLIDIPVSNCLNSNISKIYVLTQFNSASLNRHLSRAYASNMGGYKNEGFVEVLAAQQSPENPNWFQGTADAVRQYLWLFEEQNVLEYLVLAGDHLYRMDYERFIQAHRETDADITVAALPMDEKRATAFGLMKIDEEGRIIEFAEKPKGEQLKTMKVDTTILGLDDARAKEMPFIASMGIYVISKDVMLSLLRDKFPGANDFGSEVIPGATSIGMRVQAYLYDGYWEDIGTIEAFYNANLGITKKPVPDFSFYDRSSPIYTQPRYLPPSKMLDADITDSVIGEGCVIKNCKIHHSVVGIRTCISEGAIIEDTLLMGADYYETDADRRLLAAKGSVPIGIGRNSHIKRAIIDKNARIGEDVKFSYLPLDNIHSSQIVNGDNVQEAARETDGYFIKSGIVTVIKDALIPSGTVI

SEQ ID NO: 106 - DNA, 4197 bp, Citrullus lanatus
  source 1..4197: mol_type = genomic DNA
atggccgtctcttccggttgccgggccgcgcatgtaagggattcacctgccacttcagtggtcgtcgcgaggaagtttagatctgtcgagtttggcaatggcgaattcatggggaagaaactcacggaaccgaaacgagtacccgctgctgttgttaaaagtgttggactacgtgtttgcatgtctctcacgactaatgtagccattggcgacgctgcggtaatgttgagaattctgcagagtttgaacttgccgatatttgtttttgtttttcttgcgttgtgaattttgaaatgccttttgtttaaatcggtaatttgttgagatttgaaactacacctggtgttgcttcacattgatttctggaatttgtttgttaacatgttgaactgcaaaggcttcgtcttggtgatgaatcgttgatctgtctctgtatttcgataaaatttcctctgtaggacgttaatctcctagatatggtttgaacttcagagaagactaattaggatgccgattaagttgccaactactatggcatttaatcattcgaatcttttgtttagtactctacgccttctggttccagttgaatctaacaacttactgcaatttggccttaatccgatctttatatgtatgaacaaacgtgagaaataccattgggaatggaatcaagggactgaataattgtattgatctggcgcaataattaatatgttgttgcttgtgactttgtttagttgagagaaatagatatggaaaaaagggaccctcggactgtggtggcgatcattctgggaggaggagctggcactcgtctattccccctcaccaagcggcgggccaaacctgctgtaagtttcctctactaaactcaatttttctttcagttatattatatcatttaaggttgattttgtttagatgaatgtaatctttctccccccgtttagtcaacgggggagaaagttgtctccgctccctcctccattttccgtctaatcggaaattcgccatctcgttagggggtggattctacggagtaattggatatctgtaatttaatgacattttattttcatatatatccatgtgggattttgttataacggctgccctctgctttcttatgatttcccattacaggttccaattggaggagcatataggctgattgacgtgccaatgagcaactgtatcaatagtggaattaataaagtctatattctcactcagttcaattcagcatcattaaataggcatcttgctcgagcttacaatttcagcggtggagtcacctttggagatggctatattgaggtatgtttccttcacttaaatcatgcctagaattaagtgaatcatatatccacaatcatattcccaaaccaaatatgacttatagagctttcagctatatctcaggttcttgcagccactcaaacgccaggggaggctggtaaaaattggtttcaaggtacagcagatgccgtgcgacaattccactggctttttgaggtacaaaaaaaccgctatcatgttaaataatgtgaattatgatattgagaagaatataaagaaatagttgttcgtgacattttggttgttccatatacattattcaacataaaaataggacaaagtgacgaaaggaaaaattgaatgttaaatatgtatgtacgttccttggggtcatatctgacgtagaaaccaacctacatgtaaggagtgagatagtaataagtctttcctgctgcaggatgccagaagcaagaatattgaagatgtgctgattctgtctggagatcacttgtacagaatggattatatggatttcgtccaggtgaggctgaaaatgcatttaagaaccaatcttcatctgttgtttacattcatttctgatacatcttctttacattggcagaatcataggcaaagtggtgcagatatcactatttcttgtctgcccatggacgataggtgagtgacaacttgatgttttccttttggaaggttagctaaaaaatcattaatcctgaaaggccccacagaccttgatcaatgcaatcagttcaaacctttttctgttcacagaaatttgagtacctaggtcttaaggttcaaactattacagaaatgtgtaaagaaaggagaaagagcatctttacttcaaatttatgtttcttctaactgaaagcagaagagcatctaatgctcctgcaatttatttaaaaatcacattaccttttattatagccgagcctcagactttggtctaatgaagatagacgacaaaggaaggataatttcattcagtgaaaagcctaaaggacaggacctgaaggccatggtaacattcaaaaattgccttcactagaaactttaaatcagtggaggaacttaaatgattgttctggtgaacctaaactgtatcatctatattcaaaatgaaggcagtagatacaaccattttgggactctcgaaagaagaggctcagaagaagccatatattgcttccatgggagtgtatgtctttaagaaggagatacttttgaatcttttaaggtattgtgcatctaatgccatttatgaatctagtgattttatataaacttaaaggactttgtcgaacacaaaatattagactttgcttgaatttgttcaagtcagatggcgctttccaactgctaatgactttggatcagagataatccctgcctcagccaaagaattctttataaaggtttgtaatttgtacgtaatctgtccgtcgagactgttgtatcatttttttttcccaactttctactcgcaatttagcaacaaactaatgctagcctcaataattctgcacaggcttatctcttcaatgattactgggaagacattgggacaattagatccttctttgaagcgaaccttgccctgacagagcaagtaaggcaactaaacatttttcatttacaatttcttattcttgctctcaccctttaggactttctcaaattactagaattgtgatttgacctttttatcttgttaatcttgctgttgcacaccataggtctagtctcatgtttgctttatgcctttcagccaccaaggtttagcttttatgatgccgcaaaaccgatgtttacttcaagaagaaacttgccaccttcaaagattgaccagagcaaggtcagtagtattctgcagtcatccaagtctctattataaaatctaacacagcattttggcaaaaaaacaaaccagctgagcattcttctggtacctgccaatttttctttgctttttatgttgagataatatttgggtcctttgcagatcgtagattctatcatctcacatggaagcttcttaaacaaatgctttatagagcacagtgtcatcggcatccggtccagaatcaactctaacgttcacttgaaagtatgtatttcatggaattttttcatgatatgctatagtgattagaaaaatatttatgaaaatttgtcattcaactataaattattatttttttgttattttaaaagaagatacttctttatacaatataaacatatcagatttctgtaacatgatggttaacatagaactgaatttcttacaaaaaaaataaaactgagttcaatccaatcctttgcaaaacgtaagttctttcttacacagagtgatttgtgatgagatgcataatgcaggacactgtgatgcttggagcagactactatgaaactgaagaagaagttgcatctttgttagctgaaggaagagtaccaataggaataggggaaaacacgaaaataaagtatgaacaccacacccaaaaaagataaccccaagatcttcatatgttctggagcctgcttttctaattcctcattgttgatataaaatattgtgtgcagggactgcattatcgacaagaacgctcgaattggaaagaatgtggtgattgccaattcagaggtaactatagcagtaggagaattcgaaatacagaccttttagtatgctagttaagctatgttcattttagctaactatattcgttgaataaattatgtgttattggaaccatattgcagggcgtacaagagggggatagatcatcagaaggcttctacatacgttctggcattaccatcatcttaaaaaatgcagtgattgaagatggtttggttatataa

SEQ ID NO: 107 - DNA, 1563 bp, Citrullus lanatus
  source 1..1563: mol_type = genomic DNA
atggccgtctcttccggttgccgggccgcgcatgtaagggattcacctgccacttcagtggtcgtcgcgaggaagtttagatctgtcgagtttggcaatggcgaattcatggggaagaaactcacggaaccgaaacgagtacccgctgctgttgttaaaagtgttggactacgtgtttgcatgtctctcacgactaatgtagccattggcgacgctgcgttgagagaaatagatatggaaaaaagggaccctcggactgtggtggcgatcattctgggaggaggagctggcactcgtctattccccctcaccaagcggcgggccaaacctgctgttccaattggaggagcatataggctgattgacgtgccaatgagcaactgtatcaatagtggaattaataaagtctatattctcactcagttcaattcagcatcattaaataggcatcttgctcgagcttacaatttcagcggtggagtcacctttggagatggctatattgaggttcttgcagccactcaaacgccaggggaggctggtaaaaattggtttcaaggtacagcagatgccgtgcgacaattccactggctttttgaggatgccagaagcaagaatattgaagatgtgctgattctgtctggagatcacttgtacagaatggattatatggatttcgtccagaatcataggcaaagtggtgcagatatcactatttcttgtctgcccatggacgatagccgagcctcagactttggtctaatgaagatagacgacaaaggaaggataatttcattcagtgaaaagcctaaaggacaggacctgaaggccatggcagtagatacaaccattttgggactctcgaaagaagaggctcagaagaagccatatattgcttccatgggagtgtatgtctttaagaaggagatacttttgaatcttttaagatggcgctttccaactgctaatgactttggatcagagataatccctgcctcagccaaagaattctttataaaggcttatctcttcaatgattactgggaagacattgggacaattagatccttctttgaagcgaaccttgccctgacagagcaaccaccaaggtttagcttttatgatgccgcaaaaccgatgtttacttcaagaagaaacttgccaccttcaaagattgaccagagcaagatcgtagattctatcatctcacatggaagcttcttaaacaaatgctttatagagcacagtgtcatcggcatccggtccagaatcaactctaacgttcacttgaaagacactgtgatgcttggagcagactactatgaaactgaagaagaagttgcatctttgttagctgaaggaagagtaccaataggaataggggaaaacacgaaaataaaggactgcattatcgacaagaacgctcgaattggaaagaatgtggtgattgccaattcagagggcgtacaagagggggatagatcatcagaaggcttctacatacgttctggcattaccatcatcttaaaaaatgcagtgattgaagatggtttggttatataa

SEQ ID NO: 108 - protein, 520 aa, Citrullus lanatus
MAVSSGCRAAHVRDSPATSVVVARKFRSVEFGNGEFMGKKLTEPKRVPAAVVKSVGLRVCMSLTTNVAIGDAALREIDMEKRDPRTVVAIILGGGAGTRLFPLTKRRAKPAVPIGGAYRLIDVPMSNCINSGINKVYILTQFNSASLNRHLARAYNFSGGVTFGDGYIEVLAATQTPGEAGKNWFQGTADAVRQFHWLFEDARSKNIEDVLILSGDHLYRMDYMDFVQNHRQSGADITISCLPMDDSRASDFGLMKIDDKGRIISFSEKPKGQDLKAMAVDTTILGLSKEEAQKKPYIASMGVYVFKKEILLNLLRWRFPTANDFGSEIIPASAKEFFIKAYLFNDYWEDIGTIRSFFEANLALTEQPPRFSFYDAAKPMFTSRRNLPPSKIDQSKIVDSIISHGSFLNKCFIEHSVIGIRSRINSNVHLKDTVMLGADYYETEEEVASLLAEGRVPIGIGENTKIKDCIIDKNARIGKNVVIANSEGVQEGDRSSEGFYIRSGITIILKNAVIEDGLVI

SEQ ID NO: 109 - DNA, 3757 bp, Citrullus lanatus
  source 1..3757: mol_type = genomic DNA
atggattcctttggtgtggcattgaagcccaatacgatgccatttagaatcagcagccagtgtgttaagaatcaatgctctgtgttctggggagagagtgttgggagaagcggaatgtgcaaacagatacagaggaatgcctactcgcggaagaattctaattcctctcctcgtagcacggctagaaagcttacaccgggagttgcctattctgttctcatgtcagaaattagtgaggaaacaacggtaagtggatttagtatttttgatgatattcaaaagtatatcaacttttacatgaattttgactccaaattttatctcagactttacaggctcctatattcgagactccaagagctgatccgaaaaagattgcttcaatcattctgggtggaggtgctggaactcgcttgtttcctcttaccagccagcgagccaaaccagcggtaaccacctctaccaaattgctaagagaaggataatttcatagtagtacttgataaaataaactattgatccaggcaaggagaacgaattagtgcacgaatttaagtaaataatatttccttgcaggttcctattggaggttgttataggttgatcgacatacccatgagtaattgcatcaatagtggcattgaaaagatattcgtcctaacgcagtttaactcattttcactcaatcgtcaccttgctcgtatttataattttggtaatggagtgaattttggcgatggattcgtggaggtatgatgactttgtttttcccattgctacaatctggccccgcttatcaattgtgttaattagacagactgcaagttctaggaaccaggaagttttcttatactccatctctggatccatctttgagaaacaggttctagctgccactcagacttcgggtgaaactggaaaaaaatggttccaaggaactgctgatgctgtcaggcaatttatatggttatttgaggtgggtgctcctcagcatggttggaattttcagtaaacttttttaatgtgtaacgaacgttttgtgcattgggtttgtgcaggacgctaagaccaaaaatgttgagcacactctaattttgtctggcgatcacctttaccgaatggactacatggattttgtgcaggtttaacgtcaaactaactagtttgtggttgtatttgtatcgttttgcacatttgtttgtgattcaatgagactttctgatggctttgtatttgcagagacatattgacacaaatgcagatattacagtttcatgcatacccatggatgataggtaggcatctttcttttttttttttttttctttttttttttttttccatcgttattttcatagataaagtgcatggaaaccataatcttattcttttatttccttaaaaacttagatttcattatatgctttgcctatttgcagccgggcatcagattacggtctcatgaaaatagatgatacaggacgcattttacattttgcagagaaaccaaaggggtctgacctggaagcaatggttggtaacattgatgcaattatagttattttattgaacttcttttgttttttgtctttttttatttgtttggttatgttatagcgtagatatagttattgactcctgttcctatagtgcccaatatctttgtctgatcttgttttctatccatcatatattccactgcagaaagttgacactacagttctaggactttcaaatcaagacgctaggaaaaatccttatattgcatcaatgggggtctacatattcagaactgatcttctattaaagcttttgacatggagttatccttcatgcaatgacttcggctcagaaattattccatcggctgttaaggactacaaagttcaagtgagatcccttttgagaaattttaatctctcattttactttccgcttgaattcagaaaaggaatactttcaacgatcatagaactaagtttctcaatgccatccatattgttgtaggcatatttattcaatgactactgggaggacattggaaccgtaaagtcattttttgatgctaacttagcgctaacggagcaggtatgtatcccatcatgcaaccagttgtgtcactggaacctatttcaggccatcgattgtctcctttcttttccattttatgggatcaagaaacatcttacaccattttttgttccagctttcatgcttatttattcattgagttagatatttacgtaggtagatgataagttcccagattatatacttctgctcatatcctcctgaagttactgatcagataccacccttaatagttaatccacatcaagtaccttaacaggcctgtgacaatttcaaccagccccctcccacctaatcagaaacaacaaatgatcaagtatagtgtaaccaatgcaaggtctttctccaaacactagtatttatacaagctgatggtttgcagtatctttcatgcagcccccaaagtttgagttctatgatccgaagactccattttatacatcacctcgattcttgcccccttctaaagttgaaaaatgcagggtaagacatcaaactcaagttttatctctgcatcataggcttgattcttgtgtcttaaatgaagctgtatttgcagattgtagatgcaataatatctcatggttgcttcctccgagagtgtagtgttgaacactctattgttggcgtgcgctctcgattggagtacggtgtggagcttaaggtgagccatggttttcttgtggatttttccctcccttcctataccataggaatgtacaagaagactaaatgcagttgcagaggctcggttgtagatgctgcattcattttccacatattaaactctttttattaatgcaagagagtctgtcgtgctgattcgtgctgattcgtattgattcgtgatagtagatggatgaatccaaattcaatgcgtactagctgttgccacatagcttcatatcttggtaaacgtcgatcatatttcgatttgttggttttgcattataggatacaatgatgatgggtgcggactactaccagactgaatctgaaattgcatctctgcttgccgaaggaaagatcccaattggcattggagagaacaccaagatcaggtaatattcagctaccaagacagccaaaaattgtctgcaatcttttactagttaaatagaactgtcagtttatcatcttactgactaccattaaaattatttgttcatgggtttggtctcgtttctattgtaacttttctgcaggaattgcataattgacaagaatgccaggatcggaagtaatgtggttattgcggaagaaatgtggttattgcaaattctgatgtaagtaataatttttgtacttgtaggggacgtttggctcatagttttagtatgtaagtttggtttacaaggttaaaatcgttttaacctggtggatttcatatatgacagcttatatgacttgagttcatgtaattcgtaggattaacagtttgggatgaccaaagcgacatgtttatcaaacatcaatttgactttttaagcacaagttaaacgagtttagcccaaaaattaaacactttccaaaaatttgcaaactcaatttaaactaaataaacgcttgatgttatttgatcatagtaaccatgcctaataaagtgtacttgtaacttttcaggacgttcaagaagcagatagaccagaagacgggttttatataa

SEQ ID NO: 110 - DNA, 1626 bp, Citrullus lanatus
  source 1..1626: mol_type = genomic DNA
atggattcctttggtgtggcattgaagcccaatacgatgccatttagaatcagcagccagtgtgttaagaatcaatgctctgtgttctggggagagagtgttgggagaagcggaatgtgcaaacagatacagaggaatgcctactcgcggaagaattctaattcctctcctcgtagcacggctagaaagcttacaccgggagttgcctattctgttctcatgtcagaaattagtgaggaaacaacgactttacaggctcctatattcgagactccaagagctgatccgaaaaagattgcttcaatcattctgggtggaggtgctggaactcgcttgtttcctcttaccagccagcgagccaaaccagcggttcctattggaggttgttataggttgatcgacatacccatgagtaattgcatcaatagtggcattgaaaagatattcgtcctaacgcagtttaactcattttcactcaatcgtcaccttgctcgtatttataattttggtaatggagtgaattttggcgatggattcgtggagactgcaagttctaggaaccaggaagttttcttatactccatctctggatccatctttgagaaacaggttctagctgccactcagacttcgggtgaaactggaaaaaaatggttccaaggaactgctgatgctgtcaggcaatttatatggttatttgaggacgctaagaccaaaaatgttgagcacactctaattttgtctggcgatcacctttaccgaatggactacatggattttgtgcagagacatattgacacaaatgcagatattacagtttcatgcatacccatggatgatagccgggcatcagattacggtctcatgaaaatagatgatacaggacgcattttacattttgcagagaaaccaaaggggtctgacctggaagcaatgaaagttgacactacagttctaggactttcaaatcaagacgctaggaaaaatccttatattgcatcaatgggggtctacatattcagaactgatcttctattaaagcttttgacatggagttatccttcatgcaatgacttcggctcagaaattattccatcggctgttaaggactacaaagttcaagcatatttattcaatgactactgggaggacattggaaccgtaaagtcattttttgatgctaacttagcgctaacggagcagcccccaaagtttgagttctatgatccgaagactccattttatacatcacctcgattcttgcccccttctaaagttgaaaaatgcaggattgtagatgcaataatatctcatggttgcttcctccgagagtgtagtgttgaacactctattgttggcgtgcgctctcgattggagtacggtgtggagcttaaggatacaatgatgatgggtgcggactactaccagactgaatctgaaattgcatctctgcttgccgaaggaaagatcccaattggcattggagagaacaccaagatcaggaattgcataattgacaagaatgccaggatcggaagtaatgtggttattgcggaagaaatgtggttattgcaaattctgatgacgttcaagaagcagatagaccagaagacgggttttatataa

SEQ ID NO: 111 - protein, 541 aa, Citrullus lanatus
MDSFGVALKPNTMPFRISSQCVKNQCSVFWGESVGRSGMCKQIQRNAYSRKNSNSSPRSTARKLTPGVAYSVLMSEISEETTTLQAPIFETPRADPKKIASIILGGGAGTRLFPLTSQRAKPAVPIGGCYRLIDIPMSNCINSGIEKIFVLTQFNSFSLNRHLARIYNFGNGVNFGDGFVETASSRNQEVFLYSISGSIFEKQVLAATQTSGETGKKWFQGTADAVRQFIWLFEDAKTKNVEHTLILSGDHLYRMDYMDFVQRHIDTNADITVSCIPMDDSRASDYGLMKIDDTGRILHFAEKPKGSDLEAMKVDTTVLGLSNQDARKNPYIASMGVYIFRTDLLLKLLTWSYPSCNDFGSEIIPSAVKDYKVQAYLFNDYWEDIGTVKSFFDANLALTEQPPKFEFYDPKTPFYTSPRFLPPSKVEKCRIVDAIISHGCFLRECSVEHSIVGVRSRLEYGVELKDTMMMGADYYQTESEIASLLAEGKIPIGIGENTKIRNCIIDKNARIGSNVVIAEEMWLLQILMTFKKQIDQKTGFI

SEQ ID NO: 112 - DNA, 3988 bp, Citrullus lanatus
  source 1..3988: mol_type = genomic DNA
atggttgcgatggattcctgctttgtgtctttgaaatccaatacccatttgatgaagggtaattggggaggtctggatcgttgtgagaatggattttatggtgagaaagttagaggaagttttaatgaaaatgcttggattaagagcttgaaatccgagaagaaagctttgaagcttacccctaatgttgcctatgctgtggccacgccaaatatttccaagcagcctgtggtgagtttaatgcattggtggatggtcatgtttaattcatatctttggaagttttgacgaaagccccatcttggtttttcttcatttttcacttttttgaattggatttgattttgcagagcattcaagtgccatcaattcccaaagtaaaagcaaatcctaaaaatgttgcttcaatcatattgggagggggtgctgggactcacctgtttccccttactagaagatcagcaacacccgctgtaaggaactaaacttgtttatatatatatatttgattgtggaactttatatggatccaagggcttaattttgatcgttttcaaagttctctgtttttgttctcactgatgttaatgtattccttagaatatatgaagctgttcaatcatttctttcaaaatcatgtattgaaggcaggttttcctaataaattttgccctttttttttctaatggtgtagtgtaatcatatatatagtccaagttattcacaatcagatcttttatttttttctgttacactaagcggttggctaatttatacaatttaaccatcaaatatttactcacatgggtaggttccagttggaggatgctataggcttatagatattccaatgagcaactgcatcaacagtgggatcaacaaaatatttgtgcttactcagttcaactctgcttctttgaatcgtcatatttcacgcacatactttggaaatggtgtcaacttcggagaaggatttgtggaggtatggtatggcgatgtgttaggaagctatcgaaaatgctcgaatcgataatctaagaacttaatgataaaaagattggaatcaaaacacaatttacagatagagttgatctttttcctagctcgactgatcgttatgatcctttgaactgactgttttattggaaaacgagagtgtaaaactgtaactattttcagtagttaattatttgtgcttctccaaattatgtgatgatgttgagcttactctcgataaacaggttcttgccgccacccaaacatctggggaaaccggcatgcactggttccaaggaactgcagatgctgtgagacagtttatttgggtatttgaggtgcaaaacaaattcttttctcttttgttctgtttgaaacttcactgtattgatattttattaagttgactttcatattaattcttgtaggatgcaaagaacagaaatgttgagaacattctaattttggcaggggatcacatgtatagaatggactatatggactttgtgcaggtaataaatttctttttgaacccccattcttattaaagataatagttctcattctaggtttcaaaaacctaattgtatgctttcattggacacagaatcacattgacagaaacgctgatatttcaatctcgtgcgcagctgtgggcgacaggtcttatcaatgttccttttaccttatgtttcttcttttaaattttttttgcttgtaggtaatagggaaagatgtgataagttcgattgtttatctaagttaacttcaaaaccgaattttgtatgcttaaacagcaatgaaagtgaaactcttatagatcacaggaatttatttatttattctttcgaattttagagccaaagtctcatatctttttaatccactctctgcagccgcgcatcagactacggattggtgaaaatagatagtagaggtcgaattatccagttttctgaaaagccaatgggtgccaatttgagtgcaatggtaaaaactacagaaaacatagaatggttactttttccactttgtgaatgaaatattgatttaagacatgcatgccattaaaacaatcattcttgggtgaaataggttcatgtatactaaaccttctcttcaatcattattgtttctcaaactacactcctaattttagcatcactttttaacttgtaatttgaatcttgttgaagtgacataatcatcccaattggttggtttcatgcagcgagtagatacaacttcatttggtttgtctcgggaggagtcattgaagtctccttacattgcgtcgatgggagtttatgttttcaaaacagatattttgctaaaccttttgaagtggagatatcctacatctaatgactttggctctgaaatcattcctgcagctgtaaaggagcacaatgtccaagtaagatgattttctatcaactctaaacgattctccattacttaaatttgtcaaggtaataatagtgtcaaacctactaatagccaataaattcgaaactcctgtgttttttaatctttgattaatatcttgaacccataaacttttatatttctgtggtagtggttctttatacaaaatgctgcataacttaagttcacctggaatcaagttcgagcgctagggacctttttttcagctgtaaataagaaataactcctttttctacttccctttctcttctatcatggaacctatttgaatacttcatatttcaacttcatttaagccattgttttcgcatggatatatgcatgtgtgagtgtaacttctatttctttcttcttctttgttttcttattataaaggcatatatatttagagactattgggaggacattggatcaataaagaccttctatgatgcaaacttggccctcacggaagaggtacatgacatttcgtggaattgaatcgtttttggttctgattttcctttactgtcgtataatcggcctgtatagtttattacttgaatttgcaatctgtttcttaagaatctgaactgtggaattaaccgaaggccacgattcttctttgctacaagcaatgtaatctgtgttgaaaggattcgttttgtaaacttactgacgttgccaattgcttcacttgagttggtattgattatcatattttcactttctcgcagtttcctaaatttgaattttatgaccccaagacacctatctatacttctcctcgattcctaccgccgaccaagattgacaagtgtcaggtatatcaataaaaccaatcagattcaaagctcatctttaggactcgactctaagctttcagttgagttttgcagattgtcgatgcaataatctcgcatggatgctttttgagagaatgtagtgtccaacattcaatagttggtgaacggtcaagattagactacggtgttgaacttaaggtgaaatcaaagaattcatagtttcagacaatgctacaggttgttggacgaacacttggatttgtaacagaggttgctgtcttcttttttacaggatactataatgatgggtgcagatacttaccaaactgaacctgaaatcgcaggtctgctagcagagggaaaagttcctataggtattggacgaaacacgaaaatcaggtaagtggactcaaaccttgaaaaatttgttagacatagatctcaacgagaatcttccaatccaacgatgttgatgggtatcatgatgggggggtttgttgcaggaactgtataatcgataagaatgcgaaaatcggcaaggatgttgtcattatgaacaaagaagtaagcatatccaacccgtctcagcaataggcattcagtcccgaaaaccccagtattctctttgtattaatctgtttctctgtcaattccagggcgtccaagaagcagatcggcctgaacagggattctacattcggtcgggaatcaccattatactggaaaaggcaacgatcgaagacggcacagttatatga

SEQ ID NO: 113 - DNA, 1581 bp, Citrullus lanatus
  source 1..1581: mol_type = genomic DNA
atggttgcgatggattcctgctttgtgtctttgaaatccaatacccatttgatgaagggtaattggggaggtctggatcgttgtgagaatggattttatggtgagaaagttagaggaagttttaatgaaaatgcttggattaagagcttgaaatccgagaagaaagctttgaagcttacccctaatgttgcctatgctgtggccacgccaaatatttccaagcagcctgtgagcattcaagtgccatcaattcccaaagtaaaagcaaatcctaaaaatgttgcttcaatcatattgggagggggtgctgggactcacctgtttccccttactagaagatcagcaacacccgctgttccagttggaggatgctataggcttatagatattccaatgagcaactgcatcaacagtgggatcaacaaaatatttgtgcttactcagttcaactctgcttctttgaatcgtcatatttcacgcacatactttggaaatggtgtcaacttcggagaaggatttgtggaggttcttgccgccacccaaacatctggggaaaccggcatgcactggttccaaggaactgcagatgctgtgagacagtttatttgggtatttgaggatgcaaagaacagaaatgttgagaacattctaattttggcaggggatcacatgtatagaatggactatatggactttgtgcagaatcacattgacagaaacgctgatatttcaatctcgtgcgcagctgtgggcgacagccgcgcatcagactacggattggtgaaaatagatagtagaggtcgaattatccagttttctgaaaagccaatgggtgccaatttgagtgcaatgcgagtagatacaacttcatttggtttgtctcgggaggagtcattgaagtctccttacattgcgtcgatgggagtttatgttttcaaaacagatattttgctaaaccttttgaagtggagatatcctacatctaatgactttggctctgaaatcattcctgcagctgtaaaggagcacaatgtccaagcatatatatttagagactattgggaggacattggatcaataaagaccttctatgatgcaaacttggccctcacggaagagtttcctaaatttgaattttatgaccccaagacacctatctatacttctcctcgattcctaccgccgaccaagattgacaagtgtcagattgtcgatgcaataatctcgcatggatgctttttgagagaatgtagtgtccaacattcaatagttggtgaacggtcaagattagactacggtgttgaacttaaggatactataatgatgggtgcagatacttaccaaactgaacctgaaatcgcaggtctgctagcagagggaaaagttcctataggtattggacgaaacacgaaaatcaggaactgtataatcgataagaatgcgaaaatcggcaaggatgttgtcattatgaacaaagaaggcgtccaagaagcagatcggcctgaacagggattctacattcggtcgggaatcaccattatactggaaaaggcaacgatcgaagacggcacagttatatga

SEQ ID NO: 114 - protein, 526 aa, Citrullus lanatus
MVAMDSCFVSLKSNTHLMKGNWGGLDRCENGFYGEKVRGSFNENAWIKSLKSEKKALKLTPNVAYAVATPNISKQPVSIQVPSIPKVKANPKNVASIILGGGAGTHLFPLTRRSATPAVPVGGCYRLIDIPMSNCINSGINKIFVLTQFNSASLNRHISRTYFGNGVNFGEGFVEVLAATQTSGETGMHWFQGTADAVRQFIWVFEDAKNRNVENILILAGDHMYRMDYMDFVQNHIDRNADISISCAAVGDSRASDYGLVKIDSRGRIIQFSEKPMGANLSAMRVDTTSFGLSREESLKSPYIASMGVYVFKTDILLNLLKWRYPTSNDFGSEIIPAAVKEHNVQAYIFRDYWEDIGSIKTFYDANLALTEEFPKFEFYDPKTPIYTSPRFLPPTKIDKCQIVDAIISHGCFLRECSVQHSIVGERSRLDYGVELKDTIMMGADTYQTEPEIAGLLAEGKVPIGIGRNTKIRNCIIDKNAKIGKDVVIMNKEGVQEADRPEQGFYIRSGITIILEKATIEDGTVI

SEQ ID NO: 115 - DNA, 4913 bp, Cucurbita moschata
  source 1..4913: mol_type = genomic DNA
tccactcaaatctcataaaattagaaataatattaatttccttttaattttattggggaaccgctcgaattttctttttccccatattgcccctatcaggaccacgttctgcgtctagaaaaccgagggtaaacgacgtaataaagtggaaatatgaagggtaaaatcgacactgagaccacatcaaatatcagtggcaaagcggttccctcattcggcagttgagctgcgagctaatctctagttttttgtttttcccgggcactgtctgatcggtgaacaaaccggatttcgctttgtgaatttggattgattctccaatggcgtcttctattgctgccggtggagttctcagagtctcgacgacgtcggggtcttcgtcgtcgaacggcggttctaaccgtgtaaggggaagcagtgttcgaagcctctcgttcggtgcgtctctcatttctggtgacaagatcgactttagaggttctgctttgggatcacgacgtgtttcaggtggtagagcggctcctttgatcgtctctccgaaagcggtttctgattccaagaactctcagacttgcctcgatcccgatgctagccgggttggtattcttggtttctttgtgttgttctctgaatgcgttaatcgattggaatcgaatagatttattgcctgttgtacttgatgcgttcgcggagtttatttcgtttgatttcctgtattgagccgctgcgatttgtttctagaatggatggtgatttaatggcgtctgttatctgtttttgtgtggttagagtgtgcttgggattattctgggaggtggtgctggaacgcggctctatccgctcactaagaagcgggcgaagcctgctgttcctcttggagcaaactacaggctgattgacattcctgttagcaattgtctcaacagcaacatttccaagatttatgttctaacgcaattcaattctgcgtctcttaatcgtcatctttcccgagcttatgcgagtaacatgggcggttacaagaatgaaggattcgttgaggttcttgcggcccagcagagtccggagaatccaaattggttccaggtgatggaattaaaattaggtttattctttcataattcatgcgaaaattggaatccctgaaaccttccattttgttcagaacattgatcttgtgatatactctgtcttaacacttccgatttcctaatgcattttctttaagtttgtcttgatctgatatactccgtcttatctcataaatgtttggcattgaaacctgcagggaacagctgatgcggttaggcagtatttatggctatttgaagaacacaatgttttggagtatctagttcttgctggcgatcatttgtaccgaatggattacgagagatttattcaagcacacagggagactgatgcagatatcactgtagctgcacttcccatggatgaaagccgtgctactgcatttggtctgatgaaaattgatgaagagggacggataattgagtttgcagagaagccaaaaggagaccaattgaaagcgatgcaggtgaatccaaactccaatactttttatcataataaactgttctcctatgtcttgattcataagattttcttgttagatgggttagtctaaagcagaaaaaaaaaaaaaaaaatcctgttttctttcttacaggttgatactactattttgggtcttgatgatgagagagcgaaagagatgcctttcatagccagcatgggtatttacgttatcagtaaggatgtgatgctgaatcttctaagagaaaaatttccgggagcaaatgattttgggagtgaagttattcctggtgcaacttccattggaatgagggtatggatatgatgatcttatgcaccataaatatatgtaatatcaatattgatgaaagtattgtccaaatatatcatagaattcgttagtggaaagtaccgaacaacggtggccttgacaatcgagttgtcagatgcaaactctagccagtacatgggtaagatcttacctcggttggggggagaacgaaacattctttataagggtgtgaaaacctttcaccagtagatacgttttaaaaataaaaaatcttgagggaaagccccaaaagggaagtctaaagaggccccaaaagagagtctaaagaggacaatatgtgctagtgattaaagaaaataatagaaattaaaagaaaaaaaaggcaaagtagtagatgaatgagtgctgatcttgaactcatgtcacatttgattactcttggtttgctggagaacgatgacttcagagctcgtaagggctgcgatttctataataaagatattgatttgactttgtaggtgcaagcttacttgtacgatggctactgggaagatattggtaccattgaagccttttacaatgcaaatctggggatcacaaagaagccagtaccagattttaggtaatatttgattaagttattaatgaattgttaagttatcatgtgtttatttgatactcgaaacacccaattgcagcttctatgatcgttcgtctccaatctacacccaacctcggtacttgccgccgtcgaaaatgctcgatgctgatatcactgacagtgttattggtgagggctgtgtcataaaggtaacttctttacgaagctctttgctatgctaagatggtgttattacctcgtcttaactgcattattccttcccctttccatagttggaataaccccttttttattttctcctcagaactgcaagattcatcattctgtcgttggcctccgatcttgcatatccgaaggtgcaatcatagaagacacgttattgatgggtgcagattattatgaggtctgtagccaataataaccttgtttatcatgagtttcagatagctttgagctctcttggaagtacttttagattcttatacatcatgaaaatgttggaaaactattatattaaaatatagagccatctaaatgacttgtaattgtatgttgaggattgtcaggaggaaattccacattaactaatttaagaaatgatcgtgagtttataagtaaggaatatatcttcattgatataaaacttttttggaaagctcaaagcaaagctacgagaacttatgttcgaattggacaatatcatattattacgaaagtcatgcaatgtatcattcgttgaggagataaattgaggggtggttagagcataaactgatttgttgttttccttgtggtgctcctcagacggacgccgaccgtaggttattggcggcaaagggtagtgtaccaattggcattggcagaaattcccacatcaagagagctatcattgacaaaaatgctcgcatcggggaaaatgtcaaggtttgtcatgtcaacttttgtttgcttttatttcgtttttaacatttgtgttattatatcttatcatgcattttcggggtggggtcgatttccattatctttttactctcaatcaaatcccactccttccattgagaaggaacaaaggcaaatagacaagaaatgagttcataagagccttttctctctcttaaagtagttttgacggtgggaataaatctagtacaaatcagcttcacatggccgcacacgtttggtctttttttatttttttatttataatctatatattaaaaattatggtataaacttttaattaggtatctaacccgtccttaacccatgaaattgtacttacaaaaaaaaaaaaaccaaccacatttggagatgataactagttaaacacaccatttttaaatttcaaagatcaatagaatttgttgagaacttttataagtgcgtgccgagtctaaatacacatttgggttgagtgccaatgagttgccaatgagttgggtatgaatgaactgtgatataattgtctcgacatttggttgagtgtcatgagttgggtatgaatgaattgctatataattttctaactggccgattgatattcatcgcagatcgtgaacggagacggtgtgcaagaagcagcaagggagacagacgggtatttcataaagagtgggatcgtgacagtaatcaaggatgctttgattcccagtggcactgttatctaaagagagaacagaacagaacagagcagacatgtttttcattgaactggtgcctctctctctctctctctcttcctcagttttggttactgttccatttggtagtaaatgcagagtttaggggagagaatccacagtccacagtccccaccattaggcactgttctacttttaacttatgctgtaaatgttcctcccctcttcctcttcttctgcactccatcatttcattaccttccttctttctcttcatttccccccttgtaccccatttccttcaaaataatcacctattaagaacaataacagctcactttttgaatatcattctttcaaagttttctttttaattatggtccattcgtctttcgcattaaataagtaaatatagctatgagcattttaagcacaaaaaataacctccaaactaaaaagttagtagctctaaattcactggtgttagtggaaggatgtggtgtttgtttacctccattagtttgtaagggatccaatgtagactttgatagggatcaaatggatgttcatgtaactttatcattggacactcaagcagagacgcgtttacttgtgttttctcatatgaatctcttgtcttcagctctcttgtcttcagctattgaggatctgtaccaactcaagatatgcttattgggctctatgtatgtcgttcattctcatgtaaacctttttaataaataaatgaatttaattttataatagataattagaaataaaaaaataaaatcaagtttaggac

SEQ ID NO: 116 - DNA, 1575 bp, Cucurbita moschata
  source 1..1575: mol_type = genomic DNA
atggcgtcttctattgctgccggtggagttctcagagtctcgacgacgtcggggtcttcgtcgtcgaacggcggttctaaccgtgtaaggggaagcagtgttcgaagcctctcgttcggtgcgtctctcatttctggtgacaagatcgactttagaggttctgctttgggatcacgacgtgtttcaggtggtagagcggctcctttgatcgtctctccgaaagcggtttctgattccaagaactctcagacttgcctcgatcccgatgctagccggagtgtgcttgggattattctgggaggtggtgctggaacgcggctctatccgctcactaagaagcgggcgaagcctgctgttcctcttggagcaaactacaggctgattgacattcctgttagcaattgtctcaacagcaacatttccaagatttatgttctaacgcaattcaattctgcgtctcttaatcgtcatctttcccgagcttatgcgagtaacatgggcggttacaagaatgaaggattcgttgaggttcttgcggcccagcagagtccggagaatccaaattggttccagggaacagctgatgcggttaggcagtatttatggctatttgaagaacacaatgttttggagtatctagttcttgctggcgatcatttgtaccgaatggattacgagagatttattcaagcacacagggagactgatgcagatatcactgtagctgcacttcccatggatgaaagccgtgctactgcatttggtctgatgaaaattgatgaagagggacggataattgagtttgcagagaagccaaaaggagaccaattgaaagcgatgcaggttgatactactattttgggtcttgatgatgagagagcgaaagagatgcctttcatagccagcatgggtatttacgttatcagtaaggatgtgatgctgaatcttctaagagaaaaatttccgggagcaaatgattttgggagtgaagttattcctggtgcaacttccattggaatgagggtgcaagcttacttgtacgatggctactgggaagatattggtaccattgaagccttttacaatgcaaatctggggatcacaaagaagccagtaccagattttagcttctatgatcgttcgtctccaatctacacccaacctcggtacttgccgccgtcgaaaatgctcgatgctgatatcactgacagtgttattggtgagggctgtgtcataaagaactgcaagattcatcattctgtcgttggcctccgatcttgcatatccgaaggtgcaatcatagaagacacgttattgatgggtgcagattattatgagacggacgccgaccgtaggttattggcggcaaagggtagtgtaccaattggcattggcagaaattcccacatcaagagagctatcattgacaaaaatgctcgcatcggggaaaatgtcaagatcgtgaacggagacggtgtgcaagaagcagcaagggagacagacgggtatttcataaagagtgggatcgtgacagtaatcaaggatgctttgattcccagtggcactgttatctaa

SEQ ID NO: 117 - protein, 524 aa, Cucurbita moschata
MASSIAAGGVLRVSTTSGSSSSNGGSNRVRGSSVRSLSFGASLISGDKIDFRGSALGSRRVSGGRAAPLIVSPKAVSDSKNSQTCLDPDASRSVLGIILGGGAGTRLYPLTKKRAKPAVPLGANYRLIDIPVSNCLNSNISKIYVLTQFNSASLNRHLSRAYASNMGGYKNEGFVEVLAAQQSPENPNWFQGTADAVRQYLWLFEEHNVLEYLVLAGDHLYRMDYERFIQAHRETDADITVAALPMDESRATAFGLMKIDEEGRIIEFAEKPKGDQLKAMQVDTTILGLDDERAKEMPFIASMGIYVISKDVMLNLLREKFPGANDFGSEVIPGATSIGMRVQAYLYDGYWEDIGTIEAFYNANLGITKKPVPDFSFYDRSSPIYTQPRYLPPSKMLDADITDSVIGEGCVIKNCKIHHSVVGLRSCISEGAIIEDTLLMGADYYETDADRRLLAAKGSVPIGIGRNSHIKRAIIDKNARIGENVKIVNGDGVQEAARETDGYFIKSGIVTVIKDALIPSGTVI

SEQ ID NO: 118 - DNA, 5424 bp, Cucurbita moschata
  source 1..5424: mol_type = genomic DNA
ttggtagtagagttgcagagctgatctctgcttttcattttccggccaccggctcatcggagaagataccgggttttgctttctgtttttcttccgatttgaattgactgctttaatggcatcctcaatggctgcagctggagttcttagacttcccatgacggcgtcgtcggcgtcaagcggcagttctaaccgtgcaaggagaagcaaccttgggagcctttcgttctctgaatctctcgtttctggtgataaaatcgattttagagcttctggtttgggatcacgacgagtttccggtggtagagggccttctttgatcgtctctcccaaagcggtttctgattccaagaactctcagacttgcctcgatccagacgctagccgggttcgtattcttgtttctttgtggcattcgtcgttgcttattgttcttgatgcgtttccggactttacgtcgtttgattttctgttttgagccgccgcgaattgattttcgattatatgttttgtgttgtttagagtgttctagggattattcttggaggtggcgctggaacgcggctgtatccgctcacgaagaagcgagcgaagcctgctgttcctcttggagcgaactacaggctgatcgacattcctgttagcaattgtctcaacagcaacatttccaagatttatgttcttacgcaattcaattctgcttctcttaaccgacatctttctcgagcttatgcgagtaacatgggcggttacaagaatgaaggatttgttgaggttctagccgctcagcaaagtccggagaatccaaattggttccaggtgatgaaatcaagtttatatttatgctttcatgatcaatacgaatccaaattggttctctgaacgctagcattaaaacttccgaatccattttcttcaagtttctcgaaaagtgatgaaatcaagcttaaatttatgctttcatgatcaatacgaaaattggaaaaactgaaacctatcattttgttctgaaattagaacttacgaatacattttctgaaacctatcatctgatagtttgacaccataatctgtagggcacggctgatgcagttaggcaatacttgtggctatttgaagagcataatgtattggaatatctagttcttgctggtgatcatttgtaccgaatggattacgagagattcattcaagcacacagggagactgatgcagatatcactgtagctgcattacccatggatgaagagcgtgctactgcatttggtctgatgaagattgatgaagaagggcgtatagttgaatttgcagagaagccaaaaggagagcaattgaaagcgatgaaggtgaatcccaactttctatcctctcacgtttggctggttattactctcagcgaccaatctttatcattgctatgtcttctttcttacaggttgatactactattttggggcttgatgatgagagagcgaaagagatgccatacatagccagcatgggtatttacgttatcagcaaggatgcgatgctgagtcttcttagagataaatttcccggagcaaatgattttgggagtgaagttattcctggtgcaacttccattggaatgagggtaagcatataatgctcaagacaagtcataatatcatatcatccatatattttgtaaatttcttagtgggaatgtggtggcagttataaatggtgtcagagctagacaccgggtagtgtaccagagcgaggacactggccccaagagggtagattgtgagatcccacctcggttgggaagggtctggaaacctacccctagtgggcacgttttaaaaaccttggggggaggcccaaaagggaaagctcgaaaaagacaatatcggtggattgtgagatcccacatcggttgggaagagtgtggaaaacctacccctagtgggcacgttttaaaaaccttgggggcgcgaaagaaaaagctcgaaaaagacaatatctgctagcggtgggctttagttagggtggattgtgagatcccacatcggttgggaagagtgtggaaaacctacccctagtgggcacgttttaaaaaccttgggggcccgaaaggaaaagctcgaaaaagacaatatctgctagcggtgggctttagttagggtggattgtgagatcccacggttgggaagagtgtggaaacctctccctagtggacacgttttaaaaaccttgaagagaatctcaaaagagaaagcccaaagaggataatatttgatagcggtgcacttgtcctgttacaggaaagttacaagaaagtatcaaataatggaaccggtgcaaagtctaaacatttcacctatgatcatactaggattcagaagagaaaatagactaagtttagactaatgagaattctgatcatgaactcctgtcactttgactgttcttggtttgctgcagagaaatgactgtaaagctacttttatcaggccatgatctctatcacaaagtgttgctttgacattacaggtgcaagcttacttgtatgatggctactgggaagatatcggtaccatagaagccttttacaatgcgaatttggggatcacgaagaagccaataccagactttaggtaattgagtcaagttcgattcagtttccccattttgttaccaataagctgttaacttatcatttgttatttgatacacgaaacacccaactgcagcttctatgatcgttcatctccaatctacacgcaacctcgatacttaccgccatccaaaatgcttgatgctgatatcacagacagtgttattggtgagggctgtgtcatcaaggtaactcccttacaagcaaagtagctgtttgctatgctaatagcgttgttacattgtctcaattgacatcaatcttcttttgttctgtcattagagctgcaagattcaccattctgtcgttggcctccgatcttgcatatcagaaggtgcaatcatagaagacactttattgatgggtgcagattattatgaggtgtgtgaccatataataacctttttgatctttctttgaagtacttttagattcccagaagtgtttgagtttggaaaatttgcttttaatttactattaaaaaacactttttacccttgattggaccatgtttgttttgcacttagtttgttggcagtaagaactgagaagttgtgaggttttactgatttgttttactgtggtgttcctccatagacggatgccgaccgtagattattggcggcaaagggaagtgtaccaataggcattggcagaaactctcacatcaagagagctatcattgacaaaaatgctcgcattggggaaaatgtcaaggtttggcacgtcaacttgctttgtttttattcatttattttgtgatgctcatcttcaagttcttattatatcatatcattcattctcggggtggggtctcatcgggatgttgatttccactatccttttgactatcaatcaaaccctctgatttcattgggaaagaacataaagaacacagacaagtagacaagaaattgagttcatcaatgcttcctttttttcctaaagtagttttgatggtgggaacaaatctactgcacatggctgccatattcatttcttatttcaaaattaggaagctataaaaaacaacatatttactgtactaaacttttttatatttaattatgtgtctaataggttgttgttctattgaatgcttgaatcctaccaaatttaacagctataatataaacgcttgaatcctaccagacttaacagctataatatgtgctctcctattaaacacacacacacacacgcacacaacaaatccttaaattacaagggtccgtcttggaaggacttacatttcttgatataagcccaagctccccctaccaaatagttatatactgtgaaccaaatagttatatactgtgagccgttgttgttaccctcttgcttgcatatataacacctctttcaaaatctctctttcgaaaatctctttctgccctcgttttctacaactttcttcttaaaccgaggccagaggctagagcatacgtcgcttggccgtaggcggtgtaagaacaaattggcttagctcacttgttgctagaaagtgagtgccgcacaatcgcaagtccgcttgccatcaaaagtgtgattgtgaagtcttggaagtacttacatttcttgatataagcttacacttaccgctagctgatattgttttctttaggtttttcctttcgagttttccttccaagttttaagatgcttatgtcagagagagatctccactatatatcatatatttacaagggtgggaacagcagtagtacgagttcgtatgtgagtaataataataagcatatgaatgttgttgttgggtgtgtgatattgaatgaagtttgtgatctcagattgtgaatggagacggtgtgcaagaagcagcaagggagacagatgggtacttcataaagagtgggatcgtgacagtaatcaaggatgctttgattcccagcggcactgttatctaaggccacaacagaacagagcagacatgattttcactctctctctctctctctctcaattctggttcttcccctgcccactcaacattccttttcgtagaaaatgcaaagcttataggaagagaatcccacacttcacactccacactccactaggccctatcatatataacttatggtgcaatcatccctctcttctgcactcaatgtttttgttccatactacttcatgaataatcaataatcaaattctgtttcagtgattgttagtatgaataaagagcttgcttcagtgatttgatttccatcttctgtttttgaacttctttatacttttagctgttctttatcacttcaaaattaagaaacttggaatgtcattaaaccccagcaaacgaaatttgtgtggggcacctgaagtcgtatgttatacagtagtagtgatccgaatcaaattgaaaggttcctgctacaacagaacagttcaagtcatatctctatctctcatagtctacaaacttcaaaagtgttcgacaaaacttcaaaagtggacgcctttttatttggccgtttttttggatgttaaataccattggatggattactttttattggcactggaagcacatcacattatggcaaggtaaagtttcgcacagctttaatgaatcttccacttatttgatgggatggttaagagattatctatggttaaactcttcacaacttatcaatggatataatccttttggtatgaatagtttatcagtctgggtgtggctgttcttatttggacatcttgtttgggttactggatttatgttcttaatttcctagcgcggatatgccaattga

SEQ ID NO: 119 - DNA, 1617 bp, Cucurbita moschata
  source 1..1617: mol_type = genomic DNA
atggcatcctcaatggctgcagctggagttcttagacttcccatgacggcgtcgtcggcgtcaagcggcagttctaaccgtgcaaggagaagcaaccttgggagcctttcgttctctgaatctctcgtttctggtgataaaatcgattttagagcttctggtttgggatcacgacgagtttccggtggtagagggccttctttgatcgtctctcccaaagcggtttctgattccaagaactctcagacttgcctcgatccagacgctagccggagtgttctagggattattcttggaggtggcgctggaacgcggctgtatccgctcacgaagaagcgagcgaagcctgctgttcctcttggagcgaactacaggctgatcgacattcctgttagcaattgtctcaacagcaacatttccaagatttatgttcttacgcaattcaattctgcttctcttaaccgacatctttctcgagcttatgcgagtaacatgggcggttacaagaatgaaggatttgttgaggttctagccgctcagcaaagtccggagaatccaaattggttccagggcacggctgatgcagttaggcaatacttgtggctatttgaagagcataatgtattggaatatctagttcttgctggtgatcatttgtaccgaatggattacgagagattcattcaagcacacagggagactgatgcagatatcactgtagctgcattacccatggatgaagagcgtgctactgcatttggtctgatgaagattgatgaagaagggcgtatagttgaatttgcagagaagccaaaaggagagcaattgaaagcgatgaaggtgaatcccaactttctatcctctcacgtttggctggttgatactactattttggggcttgatgatgagagagcgaaagagatgccatacatagccagcatgggtatttacgttatcagcaaggatgcgatgctgagtcttcttagagataaatttcccggagcaaatgattttgggagtgaagttattcctggtgcaacttccattggaatgagggtgcaagcttacttgtatgatggctactgggaagatatcggtaccatagaagccttttacaatgcgaatttggggatcacgaagaagccaataccagactttagcttctatgatcgttcatctccaatctacacgcaacctcgatacttaccgccatccaaaatgcttgatgctgatatcacagacagtgttattggtgagggctgtgtcatcaagagctgcaagattcaccattctgtcgttggcctccgatcttgcatatcagaaggtgcaatcatagaagacactttattgatgggtgcagattattatgagacggatgccgaccgtagattattggcggcaaagggaagtgtaccaataggcattggcagaaactctcacatcaagagagctatcattgacaaaaatgctcgcattggggaaaatgtcaagattgtgaatggagacggtgtgcaagaagcagcaagggagacagatgggtacttcataaagagtgggatcgtgacagtaatcaaggatgctttgattcccagcggcactcgcggatatgccaattga

SEQ ID NO: 120 - protein, 538 aa, Cucurbita moschata
MASSMAAAGVLRLPMTASSASSGSSNRARRSNLGSLSFSESLVSGDKIDFRASGLGSRRVSGGRGPSLIVSPKAVSDSKNSQTCLDPDASRSVLGIILGGGAGTRLYPLTKKRAKPAVPLGANYRLIDIPVSNCLNSNISKIYVLTQFNSASLNRHLSRAYASNMGGYKNEGFVEVLAAQQSPENPNWFQGTADAVRQYLWLFEEHNVLEYLVLAGDHLYRMDYERFIQAHRETDADITVAALPMDEERATAFGLMKIDEEGRIVEFAEKPKGEQLKAMKVNPNFLSSHVWLVDTTILGLDDERAKEMPYIASMGIYVISKDAMLSLLRDKFPGANDFGSEVIPGATSIGMRVQAYLYDGYWEDIGTIEAFYNANLGITKKPIPDFSFYDRSSPIYTQPRYLPPSKMLDADITDSVIGEGCVIKSCKIHHSVVGLRSCISEGAIIEDTLLMGADYYETDADRRLLAAKGSVPIGIGRNSHIKRAIIDKNARIGENVKIVNGDGVQEAARETDGYFIKSGIVTVIKDALIPSGTRGYAN

SEQ ID NO: 121 - DNA, 4229 bp, Cucurbita moschata
  source 1..4229: mol_type = genomic DNA
gcgtaatggagtttccatctaccgccaatccgaattagtggttgaaattcataaaggataacatggtcctgttcttccaggtctcatgcagtccatgtgagcactcctcactcgatgcgtaactttcttctgcaacgtctaagcttctagcagtttcgaatcggtcctccatttccgagagatatcggaaaatggcggtctctgccgactgtcggattgccttatcggccgccgcgcatgtgcgggatgcacatgcggctgcggtggtggtcgggaggaagttgaggtctgtggagtttgccaatggcgagttcatgggaatgaagctcaaggtcaaggaaccgaaacgagtaggcgctgctgctaagaatgttgggcgacgtgtttgtatgtctcttaccactaatgtagccattggcgacgctgcggtaatgttgagaactctgctgagaagtattttaatcttctttatacgatttggaacattagagttcgaacttgtccagatttgttttgtttttattgcggtgtgaactttgttgtttcggtaattttgcggagattgaaactacaccttcgttgcttcgcattgatttagtttgtttgtccacattttgaaatgcaatggctccgtgttggtgatggtctgtctctgtatttcgataaattttcctagggcgtaagtctccaagatatggtttgaactttagagaagactgctaattaggatgctaatgtctagccttctacctttagaattagtaaccagtgcgatttatgtggttaccatctactatgaccttagatcattcgattctttgttcaggactctacgccttctgattccagttgaatctaacaacgtgctgcaatttggacttaacctgacctgtgtatatatttatatatatttgaacaaactcaaaatataccattgggaagtgaatcaagggactgaataatttttagaccgtcacaatcttttgtttcccccattcctaagtgagaccccttcaaaagtattgattaattgggaggagtgatatcagaaactcaccctgctttctatctcttactagtagggaaatattgtgtataattcactctcaaaatgatctgggacaacagttaatgtgttgctgcttgcgactttgtttagttgagagaaatagatacggaaaaaagggaccctcggactgtggttgcaatcattctgggaggaggagctggaactcgtctattccccctcaccaagcggcgtgccaaacctgctgtaagttttccctgctaaactcaatttttcttgctgttatttatcattgaagtattgtaactgctagccactgtgaaatcaagtgagttccagttatttttccctcttcaggttcatttagtttagatgattggaattagtgacattttattttcatatatccatatgggattttgttacattggctgctctgtgctttcttatgattttccgttacaggttccaattgcaggagcgtataggctgatcgacgtgccaatgagcaactgtatcaacagcggaatcaacaaagtctacatcctcactcagttcaattcagcatcactgaataggcatcttgctcgagcttacaactacagtggtggagtcacctttggagatggctatgttgaggtacgtttccttcacttaaaacatgtccctcaagaattaagtggatcatatatccacaatcatattcccaaagcaaaatatgactaaaggtttcagctgcatatcaggttcttgcagccacccaaacgccaggggtggctggtaaaaattggtttcaaggtacagcagatgccgtacgacaattccactggctctttgaggtacaaaaaaaaaccttatctaataacataatgatgtaagatatgatattgagaagcatataaagaaatgattgtttgtgacattttggttgttccgtatacagtaaatgatgaaaggaaaaattgggttaaatacgtattaactttccttggggtcatatctgacatagaaaacaggccttcatctaaggactgagatagtaataagccttgcctgctgcaggatgccagaagcaagaatattgaagatgtgttgattctgtctggagatcacttgtacagaatggactacatggattttgttcaggtaaggctgtaaatgcacttaagaaaccaatcttcatctgtccttacatttgtatcttctttacattggcagaatcataggcaaagtggtgcagatatcactctttcttgtctgccaatggacgacaggtgagcgacaacttaatgtttttctttcggatggttagctaaaaatcattgatcctgaaaggccccatagatcttgatcatgcaatcagttcaaacctttttctgtttgcaaaaacttgagtatctgggtcctaaggttcaaacaattacagaaatatgtaacgaaaggagaagagcatcttttcttcaaatttacttcaaacaatcacattatcttgtattatagccgagcctcagactttggtctaatgaagatagacgacaaaggaaggatactttcattcagtgaaaagcctaaaggagaggacctgaaggccatggtaacattcaaaatttccttcactagaaatttcaaatgggtggaggaacttaaaatgattgttctgatgaacctaaactatatcatttccattcatattgaaggcagtagatacaaccattttgggactctcgaaagaagaggctcagaagaagccatatattgcttccatgggagtgaacgtctttaagaaggagatacttttaaatcttttaaggtattgtgcatctaatgctatttatgaatctagtaactttatacaaactcgaaggattttgtcaaacacgaagtattagactttgcttgaatttgttcaagtcagatggcgctttccaactgctaatgactttggatctgagataatccctgcctcagccaaagaatactttataaaggcttatctcttcaatgattactgggaagacattgggacaattagatccttctttgaagcgaaccttgccctgacagagcaagtaaggcaactaaacactttccatttaaaattttctactctctctctctctctctcatcctttaagacttttctcaagttattcgaattgtagtttgacctttttttattgttattcttgcttgtttgcttatacctttcagccaccaaggtttagcttttatgatgctgcaaaaccgatgtttacttcaagaagaaacttgccaccctcaaagattgaccagagcaaggtcagtagtattctgcagtcgtctaaatctctattataaaatcatagactctaacaaaaaccttcattcctttttaacgaaatatgctgtatttggccccaaaataacacagcatttggcaattggagaaaagaaaccagatattaacaacggctgagcatttttctggttatctgacaatttttctttactctttatgttgagataatatttgggtcctttgcagatcgtagattctatcatctcacatggaagcttcttaaacagttgcttcatagagcacagtgtcatcggtattcggtccagaattaactctaacgtgcacttgaaagtatgcatctcatggaacttttgcaagtgataatcaaactaaggagttgaattcagtcctttgcaaaacataagttttttctgaacgaagtgattttttgatgagatgcaaaatgcaggacactgtgatgcttggagcagattactatgaaactgaagctgaagttgcatctttgttagctgaaggaagagtaccaataggaataggagaaaattcaaaaataaagtatgaaggccacaaccaaaaatatttttgagcctgcttttctcattcctcattgttgatataaatattatgtgcagggactgcattattgacaagaacgctagaattggaaagaacgtggtcattgccaattcagaggtaccgttaccatagagtaaaaaagggatttgttgcattcgtaaaaccaatgagatgtcaattgacctcgaaatgaatgaattatgtattaccgaaaacatattgcagggcgtacaagaggcggatagatcttcagaaggcttctacatacgttctggcattaccatcatctcaaaaaatgcagtaattgaagatggtttggttatataa

SEQ ID NO: 122 - DNA, 1581 bp, Cucurbita moschata
  source 1..1581: mol_type = genomic DNA
atggcggtctctgccgactgtcggattgccttatcggccgccgcgcatgtgcgggatgcacatgcggctgcggtggtggtcgggaggaagttgaggtctgtggagtttgccaatggcgagttcatgggaatgaagctcaaggtcaaggaaccgaaacgagtaggcgctgctgctaagaatgttgggcgacgtgtttgtatgtctcttaccactaatgtagccattggcgacgctgcgttgagagaaatagatacggaaaaaagggaccctcggactgtggttgcaatcattctgggaggaggagctggaactcgtctattccccctcaccaagcggcgtgccaaacctgctgttccaattgcaggagcgtataggctgatcgacgtgccaatgagcaactgtatcaacagcggaatcaacaaagtctacatcctcactcagttcaattcagcatcactgaataggcatcttgctcgagcttacaactacagtggtggagtcacctttggagatggctatgttgaggttcttgcagccacccaaacgccaggggtggctggtaaaaattggtttcaaggtacagcagatgccgtacgacaattccactggctctttgaggatgccagaagcaagaatattgaagatgtgttgattctgtctggagatcacttgtacagaatggactacatggattttgttcagaatcataggcaaagtggtgcagatatcactctttcttgtctgccaatggacgacagccgagcctcagactttggtctaatgaagatagacgacaaaggaaggatactttcattcagtgaaaagcctaaaggagaggacctgaaggccatggcagtagatacaaccattttgggactctcgaaagaagaggctcagaagaagccatatattgcttccatgggagtgaacgtctttaagaaggagatacttttaaatcttttaagatggcgctttccaactgctaatgactttggatctgagataatccctgcctcagccaaagaatactttataaaggcttatctcttcaatgattactgggaagacattgggacaattagatccttctttgaagcgaaccttgccctgacagagcaaccaccaaggtttagcttttatgatgctgcaaaaccgatgtttacttcaagaagaaacttgccaccctcaaagattgaccagagcaagatcgtagattctatcatctcacatggaagcttcttaaacagttgcttcatagagcacagtgtcatcggtattcggtccagaattaactctaacgtgcacttgaaagacactgtgatgcttggagcagattactatgaaactgaagctgaagttgcatctttgttagctgaaggaagagtaccaataggaataggagaaaattcaaaaataaaggactgcattattgacaagaacgctagaattggaaagaacgtggtcattgccaattcagagggcgtacaagaggcggatagatcttcagaaggcttctacatacgttctggcattaccatcatctcaaaaaatgcagtaattgaagatggtttggttatataa

SEQ ID NO: 123 - protein, 526 aa, Cucurbita moschata
MAVSADCRIALSAAAHVRDAHAAAVVVGRKLRSVEFANGEFMGMKLKVKEPKRVGAAAKNVGRRVCMSLTTNVAIGDAALREIDTEKRDPRTVVAIILGGGAGTRLFPLTKRRAKPAVPIAGAYRLIDVPMSNCINSGINKVYILTQFNSASLNRHLARAYNYSGGVTFGDGYVEVLAATQTPGVAGKNWFQGTADAVRQFHWLFEDARSKNIEDVLILSGDHLYRMDYMDFVQNHRQSGADITLSCLPMDDSRASDFGLMKIDDKGRILSFSEKPKGEDLKAMAVDTTILGLSKEEAQKKPYIASMGVNVFKKEILLNLLRWRFPTANDFGSEIIPASAKEYFIKAYLFNDYWEDIGTIRSFFEANLALTEQPPRFSFYDAAKPMFTSRRNLPPSKIDQSKIVDSIISHGSFLNSCFIEHSVIGIRSRINSNVHLKDTVMLGADYYETEAEVASLLAEGRVPIGIGENSKIKDCIIDKNARIGKNVVIANSEGVQEADRSSEGFYIRSGITIISKNAVIEDGLVI

SEQ ID NO: 124 - DNA, 3717 bp, Cucurbita moschata
  source 1..3717: mol_type = genomic DNA
atggattcgtttggtgtgaccttgaaggccaatacgatgccatttagagccagcagacattgtggtaggaaggtgcaatgctccgggttctggggagagagtattgagagaagcggaaagtgcaaacagatacagatgaatgcctactcgcggaagaattctgattcttcttcccatagcagggctagaaagcttacggcgggagttgctcattctgttctcatgtcagaagttagtgaggaaacagcggtaagtggatttagtactttgatattcaaaagtacatgaacttttatcggaacttttgactccaaattttaccccagactttacaggctcctatatttgagactcctagagctgatccgaaaaagattgcttcaatcatactgggtggaggtgccggaactcgcttgtttcctcttaccagccagcgcgccaaaccagcggtaaccacctctaccaaattgcttatataagaataatttcacaaacttggagagcgtttttaattggggagatgtatttagatggttgtagtagatacttgataatagaagctattgatccaacccaggcgaacaaattggtgcatgaatttaagcaaataatttttacttgcaggttccaattggaggttgttatagattgatcgacgtgcccatgagtaattgcattaatagcggaatcgaaaagatattcgtcctaacgcagtttaattcgttttctctaaatcgtcaccttgctcgtatttacaattttggtaatggagtgaattttggcgatggattcgtggaggtatgacgcctgtacttcccttttttacaatctgcccctgctttgcaattgttttaattagatgaacccggaagctttcttatgctcattctctggattaatgtttgagaaacaggttctagctgccactcaaacttcgggtgaaactggaaaaaaatggttccaaggaactgccgatgctgtcaggcaatttatatggttatttgaggtgggtgcctaaaatcttctgagcatgattggaattttcagtcaatttttttttatgtataatgaacgttttatccattgggtttctgcaggatgctaagaccaaaaatgttgagcacactctaattttgtctggcgatcacctttaccggatggactacatggattttgtgcaggtataacgtgaacctaactagtttgtacttgtatttgtattgtcgtgctcatttgttcgtgattcaatgaggatttctgatgacttcgtacttgcagagacatattgacacaaatgcagatattacagtttcatgcatacccatggataataggtaggcatcttttttcatcgttatttccatggataaagtgttctttaaaaacttagattttcattaatgctttgttattttgcagccgagcatcagattacggtctcatgaaaatagacgacacagggcgtattttacattttgcagagaaaccaaagggatctgacctggaagcaatggttgataacattgatgcactttgtttggtttgtaattagcaatgtctttgtctgatcttgttttctatccattatatattgtattgcagcaagttgacactacagttctaggactttcagatcaagatgctagaaaaaatccttatattgcatcaatgggagtctacgtattcagaactgatcttctattaaagcttttgacatggacttatccttcatgcaacgacttcggctcagaaattattccatcggctgttaaagactacaaagttcaagtgagatcccttttgagaaattttgatctttcatttcttccttgtgcttgaattcagaataggaagactttcgacgataatagaactaagtttcccaatgcatccatattgttgtaggcatatctattcaatgactactgggaggacattggaaccgtgaagtcattttttgatgccaacttagcactaacggagcaggtatgtttcccatcgattgtctcctttcttttcctttgtaatgggaccaagaatcatctccagttttcttgcttatttagttatctgagttaaatatttatatagaagatgagtaccttaactggcctgtgaatttttcaagcaacgccctcccccgcagcagaaacaacacataatcaagtgcaattaagctgatggctttagtgtctttcatgcagcccccaaagtttgagttctatgatccgaagactccattttatacatcacctagattcttgcccccttctaaagttgaaaaaagcagggtaagacatcaaactcgagtttgctctctgcatcatccgtttgattcttgtatcttaaacgaagttgcttttgcagattgtagatgcaataatatctcatggttgcttcctccgagagtgtagtgttgaacactctattgtcggtgtgcgctctcgattggagtacggtgtggagcttaaggtgagccagggttttcttttgaattttcccctccctttcgacactgtaggaacgtataagaagaatgcaatggcagaagatggttatcgatgctgcaatctttttttttccacagattaaactcttactattattgcaagagattctgccatgctgatatgtagcatcttagcttgaaaaaagttaatcaaatttggatttgttggttttgcgttctaggatactatgatgatgggtgcggactactaccagactgaatctgaaatcgcatctctgcttgccgaaggaaagatcccaatcggcattggagagaacaccaaaatcaggtaatattcagctaccaatacagagcaaaagctatctgcaatctcttattagttaaatataaccgtcactttatcgtccgactgtttaccataaacattatttgttcatgggtttggtctgtttctgatttacatttatcttttccgcaggaattgcataattgacaagaatgccaagatcggaaaaaatgtagttattgctaatactgatgtaagtatgatttttgtgttcttaagggatatttggttcacaatttaataagttttgtttcatccatgtttgtatttgtacaaagaaataaccaataaaaggttaggtttggttcttgggtttcataacctgggtggtttcatatgtgaaggtttatataacatgagttcatgcaattcggtaggaaggggaaaattaacactttctaagaatttgcaaactctatcaaaaccacataaacccgtgatgttatttggtcatagtacctaactagtgtgtggttgttaacttttcagggcgttcaagaagcagatagaccagaagaagggttttatataaggtccggtatcacagtcacattgaagaatgcgacaataaaagatggaaccatcatttaaatccgaagagaaagctgagagttgaaaggaagcaactgctcgaggctttcaaccgacaatgctcgacaacgagtaattggcgtatgacttcgcttcatgtaaaatgttagcttcttttcttatatgtaaaaagttgaaaggcaacacattcctgagtaagagaacaactttttatttcaatccatatattattagagactacaatattcaaagcgatttagcattagttttgggatgagcaatcctttctctgtgagggagttgtttattcttagccccatttttgttcaatttccaggactgtggctgc

SEQ ID NO: 125 - DNA, 1602 bp, Cucurbita moschata
  source 1..1602: mol_type = genomic DNA
atggattcgtttggtgtgaccttgaaggccaatacgatgccatttagagccagcagacattgtggtaggaaggtgcaatgctccgggttctggggagagagtattgagagaagcggaaagtgcaaacagatacagatgaatgcctactcgcggaagaattctgattcttcttcccatagcagggctagaaagcttacggcgggagttgctcattctgttctcatgtcagaagttagtgaggaaacagcgactttacaggctcctatatttgagactcctagagctgatccgaaaaagattgcttcaatcatactgggtggaggtgccggaactcgcttgtttcctcttaccagccagcgcgccaaaccagcggttccaattggaggttgttatagattgatcgacgtgcccatgagtaattgcattaatagcggaatcgaaaagatattcgtcctaacgcagtttaattcgttttctctaaatcgtcaccttgctcgtatttacaattttggtaatggagtgaattttggcgatggattcgtggaggttctagctgccactcaaacttcgggtgaaactggaaaaaaatggttccaaggaactgccgatgctgtcaggcaatttatatggttatttgaggatgctaagaccaaaaatgttgagcacactctaattttgtctggcgatcacctttaccggatggactacatggattttgtgcagagacatattgacacaaatgcagatattacagtttcatgcatacccatggataatagccgagcatcagattacggtctcatgaaaatagacgacacagggcgtattttacattttgcagagaaaccaaagggatctgacctggaagcaatgcaagttgacactacagttctaggactttcagatcaagatgctagaaaaaatccttatattgcatcaatgggagtctacgtattcagaactgatcttctattaaagcttttgacatggacttatccttcatgcaacgacttcggctcagaaattattccatcggctgttaaagactacaaagttcaagcatatctattcaatgactactgggaggacattggaaccgtgaagtcattttttgatgccaacttagcactaacggagcagcccccaaagtttgagttctatgatccgaagactccattttatacatcacctagattcttgcccccttctaaagttgaaaaaagcaggattgtagatgcaataatatctcatggttgcttcctccgagagtgtagtgttgaacactctattgtcggtgtgcgctctcgattggagtacggtgtggagcttaaggatactatgatgatgggtgcggactactaccagactgaatctgaaatcgcatctctgcttgccgaaggaaagatcccaatcggcattggagagaacaccaaaatcaggaattgcataattgacaagaatgccaagatcggaaaaaatgtagttattgctaatactgatggcgttcaagaagcagatagaccagaagaagggttttatataaggtccggtatcacagtcacattgaagaatgcgacaataaaagatggaaccatcatttaa

SEQ ID NO: 126 - protein, 533 aa, Cucurbita moschata
MDSFGVTLKANTMPFRASRHCGRKVQCSGFWGESIERSGKCKQIQMNAYSRKNSDSSSHSRARKLTAGVAHSVLMSEVSEETATLQAPIFETPRADPKKIASIILGGGAGTRLFPLTSQRAKPAVPIGGCYRLIDVPMSNCINSGIEKIFVLTQFNSFSLNRHLARIYNFGNGVNFGDGFVEVLAATQTSGETGKKWFQGTADAVRQFIWLFEDAKTKNVEHTLILSGDHLYRMDYMDFVQRHIDTNADITVSCIPMDNSRASDYGLMKIDDTGRILHFAEKPKGSDLEAMQVDTTVLGLSDQDARKNPYIASMGVYVFRTDLLLKLLTWTYPSCNDFGSEIIPSAVKDYKVQAYLFNDYWEDIGTVKSFFDANLALTEQPPKFEFYDPKTPFYTSPRFLPPSKVEKSRIVDAIISHGCFLRECSVEHSIVGVRSRLEYGVELKDTMMMGADYYQTESEIASLLAEGKIPIGIGENTKIRNCIIDKNAKIGKNVVIANTDGVQEADRPEEGFYIRSGITVTLKNATIKDGTII

SEQ ID NO: 127 - DNA, 4026 bp, Cucurbita moschata
  source 1..4026: mol_type = genomic DNA
caaatgttcttaagtattccttgtttgcatgcggcgtagaaatcgcggatgatggttaaaaattgggtgttgtgggaaatgcaaagaaggaagaagaagaagccgttgatccatcgagttcatttcctttttttagtataaaatcctcctgattcacagacctcttctcactcacatacatgcccttaatccaccgctttcttcgtaatctcccacgcttttcccttcttcttgccccatttcattcaatccctctgccttctcctcctccgattgcttcccttttcagtttctcaggtgatttcttgcatttttttcttttgtttctgtgatctgtacgctaaatgcttgctttgatttggggttttgctttattgggatttgaatttgattgtattttctgccgtttctgagcgattgattatttgtatttggatttggatttggattttgttttttgtgtagtgagctatggattcctgctttttgtcctgcaaacccaattcccatctgtgtctgaatcagaatggattttatggggagaaagttagaggaagtttggataataatgctttgtttaatcgcttagcaaagagtttgaaatctgagaataaagctttgaagctcacctctaatggtgctgcttctgttgttgctacactaaatattaccaaggaggctgcggtgagtttaataattgttatctcttgaagtttgatcaatacccatcttgattttgattgattcttaactgtttggattggattggattttgaagagcattcaagctccaccgtttcatacagtcaaagttaaccccaagaatgttgcttcgatcatattgggaggaggtgctgggactcatctgtttccccttactagaagattggccacacctgctgtaagttcttactaatgttggtgtgtttcttagaacaaatgaagttattttttctatcaaaatcatcgtgagatcctacgttgattggagaggaacgagtgcttgtgaggacgttggcccccaacagggtggattgtgagatcccacattggttggagaggggaacgagtgctagtgaagacgctagcccccaagagaggtggattgtgagatctcatattggttggagaggggaacgagtgaagacgctggctggttggagaggggaacgagtgctagtgaagacgctagcccccaagagaggtggattgtgagatctcatattggttggagaggggaacgagtgaagacgctggcccccaaggggggtggattgtgagatcccatattggttggagatggttggagaggggaacgagttctattcatcatcaagtactatctcgtttattagactaatcggttggttgagttatgcaatttgatcgtctactcgtttttgtaggttccagttggaggatgctataggcttatagatattccaatgagcaactgcctcaacagtgggatcaacaaaatttttgtgcttacccagttcaactctgcttcgttgaatcgtcatatttcacgaacgtacttcggaaacggtgtcaactttggagaaggattcgtggaggtatactgataaactgtgaacttaatgataaaaagagggagctaatcttttcctagcatgtcgacgatcgttatgatcctgagcttactctcgatatacaggttcttgcagccactcaaacaccggggaaatccgggatgtactggttccaaggaacggcagatgctgtgagacaatttatttgggtgttcgaggtacataacagagttatcttcttttgttctgtctgaacttctctttatcgatatcgtaaagctattcgcgtgttaattcttgtaggatgccaagaacagaaatgtcgaaaacattctaattttggcaggggatcacatgtacagaatggactatatggacttcgtgcaggtaacaacattatttttgaacgctcgtgctcattagtgataatagcttccgttctatatgtcgaaaatcatatcatatgctacgattggacacagaatcacgtggatagaaacgctgatatttcaatctcgtgcgcgcctgtggacgacaggtcttatcaacgtaccttttaccttttgattccttttttacttagaattttagagcccaactctcatacgttatttcatttactccgcaactttgcagccgcgcatcggactatggattggtgaaactcgatagtagaggtcgaattatccagttttcggagaagccaaagggtgcccatttgaatgcaatggtaaaatattgaagaacatatcgaatggttataaactatgaatctagccatacatggaaatgatctttcttggctgaataaggctcgtgcgtttaatcgccctaactgatcggttttctacagcgagtagatacaacttcattaggtttgtctcgggaggaggcgttgaagtccccttacattgcatcaatgggcgtttacgttttcaaaacggacgttttgctaaaccttttaaagtggagatatcctacatctaacgacttcggctcggaaatcattcctgcagctgtaaaggagcacaacgttcaagtaagaatcgagtccgactgctagaccttaaaagaacttttgaaccgaccgcgctttttttatttcgacttcattgatatattcatatgtgatattgcttcctttttctccaaggcatttgtatttagagactattgggaggacattggaacaatcaggaccttctatgaagcaaacttggccctcacagaagaggtagctttcatttactgaatttttcactttagtcgccattgattatcgatttttcacttttttacagtttcctaagttcgaattttacgaccccaagacgccgttctatacatcgcctcgatttttaccgccaaccaagattgataaatgtcaggtatatcaataaaaccaatctgattctcggctcgtcttaaggactctgttcttaagcttcgactgcgtcttgcagattgttgatgccataatctcacatggatgctttctgagagaatgtagtatccagcattcagtaatcggtgaacggtcaagattagactacggtgtcgaacttcaggtgaacactagagtttgtaagacacggcggctggttagacgaacacgagagtttgtaactcaggttgctgtcttctttcttacaggatactataatgatgggtgcagacaattaccaaactgaactcgaaattgcagctctgctagctgaaggaaaagtccctattgggatcggacaaaacacgaaaatcaggtaagtcgtcgactcgaaccttgaaatactgcacaacaacgtttcaacagtgttcatggcgggcttgggtttgctgcaggaactgcataatcgataagaacgtgaagattgggaaagacgtcgtcatcatgaacaaagaagtaagcgcatccaacccggttcgacaaactgactgcattcaatcccaaaacctccaggcattcttaatctgtatctttgtcaatttcagggcgtggaagaagcagatcgacccgaacacggattctacgttcggtcgggaatcaccattataatggagaaggcaacgatcaaggacggcacggtcatataaagcggtctcgaggtctcagtcttgacatgtttagaggggaagaacatgctccgtgagagctgaagaatggcatttgcagcatttaggctgagaaaaagaaggattatagtgacaaagatgaatttatagataggtttggagcaaagaaagttgctttttagtataacatataaggggagatgttaactagagattttaagtctttagtgtaattctctctaaataagagtggctactactactcacccagattcatagattatattatgtacatacaaactatatctttatgtattcttcataattgttgcctttttatcatttcattgatatttggtgacactttcatattcaaatcaatatcaaaatttttggttaatgg

SEQ ID NO: 128 - DNA, 1563 bp, Cucurbita moschata
  source 1..1563: mol_type = genomic DNA
atggattcctgctttttgtcctgcaaacccaattcccatctgtgtctgaatcagaatggattttatggggagaaagttagaggaagtttggataataatgctttgtttaatcgcttagcaaagagtttgaaatctgagaataaagctttgaagctcacctctaatggtgctgcttctgttgttgctacactaaatattaccaaggaggctgcgagcattcaagctccaccgtttcatacagtcaaagttaaccccaagaatgttgcttcgatcatattgggaggaggtgctgggactcatctgtttccccttactagaagattggccacacctgctgttccagttggaggatgctataggcttatagatattccaatgagcaactgcctcaacagtgggatcaacaaaatttttgtgcttacccagttcaactctgcttcgttgaatcgtcatatttcacgaacgtacttcggaaacggtgtcaactttggagaaggattcgtggaggttcttgcagccactcaaacaccggggaaatccgggatgtactggttccaaggaacggcagatgctgtgagacaatttatttgggtgttcgaggatgccaagaacagaaatgtcgaaaacattctaattttggcaggggatcacatgtacagaatggactatatggacttcgtgcagaatcacgtggatagaaacgctgatatttcaatctcgtgcgcgcctgtggacgacagccgcgcatcggactatggattggtgaaactcgatagtagaggtcgaattatccagttttcggagaagccaaagggtgcccatttgaatgcaatgcgagtagatacaacttcattaggtttgtctcgggaggaggcgttgaagtccccttacattgcatcaatgggcgtttacgttttcaaaacggacgttttgctaaaccttttaaagtggagatatcctacatctaacgacttcggctcggaaatcattcctgcagctgtaaaggagcacaacgttcaagcatttgtatttagagactattgggaggacattggaacaatcaggaccttctatgaagcaaacttggccctcacagaagagtttcctaagttcgaattttacgaccccaagacgccgttctatacatcgcctcgatttttaccgccaaccaagattgataaatgtcagattgttgatgccataatctcacatggatgctttctgagagaatgtagtatccagcattcagtaatcggtgaacggtcaagattagactacggtgtcgaacttcaggatactataatgatgggtgcagacaattaccaaactgaactcgaaattgcagctctgctagctgaaggaaaagtccctattgggatcggacaaaacacgaaaatcaggaactgcataatcgataagaacgtgaagattgggaaagacgtcgtcatcatgaacaaagaaggcgtggaagaagcagatcgacccgaacacggattctacgttcggtcgggaatcaccattataatggagaaggcaacgatcaaggacggcacggtcatataa

SEQ ID NO: 129 - protein, 521 aa, Cucurbita moschata
MDSCFLSCKPNSHLCLNQNGFYGEKVRGSLDNNALFNRLAKSLKSENKALKLTSNGAASVVATLNITKEAASIQAPPFHTVKVNPKNVASIILGGGAGTHLFPLTRRLATPAVPVGGCYRLIDIPMSNCLNSGINKIFVLTQFNSASLNRHISRTYFGNGVNFGEGFVEVLAATQTPGKSGMYWFQGTADAVRQFIWVFEDAKNRNVENILILAGDHMYRMDYMDFVQNHVDRNADISISCAPVDDSRASDYGLVKLDSRGRIIQFSEKPKGAHLNAMRVDTTSLGLSREEALKSPYIASMGVYVFKTDVLLNLLKWRYPTSNDFGSEIIPAAVKEHNVQAFVFRDYWEDIGTIRTFYEANLALTEEFPKFEFYDPKTPFYTSPRFLPPTKIDKCQIVDAIISHGCFLRECSIQHSVIGERSRLDYGVELQDTIMMGADNYQTELEIAALLAEGKVPIGIGQNTKIRNCIIDKNVKIGKDVVIMNKEGVEEADRPEHGFYVRSGITIIMEKATIKDGTVIS

SEQ ID NO: 130 - DNA, 20 bp, Solanum lycopersicum
  source 1..20: mol_type = genomic DNA
gcagatactacgagactgat

SEQ ID NO: 131 - DNA, 24 bp, Solanum lycopersicum
  source 1..24: mol_type = genomic DNA
gtatcgtccttacgttcccaagga

SEQ ID NO: 132 - protein, 8 aa, Solanum lycopersicum
VSSLRSQG

SEQ ID NO: 133 - DNA, 21 bp, Solanum lycopersicum
  source 1..21: mol_type = genomic DNA
gcagattactacgagactgat

SEQ ID NO: 134 - protein, 7 aa, Solanum lycopersicum
ADYYETD

SEQ ID NO: 135 - DNA, 19 bp, synthetic construct
  source 1..19: mol_type = other DNA
gtatcgtccttaccaagga

SEQ ID NO: 136 - protein, 6 aa, synthetic construct
VSSLPR

SEQ ID NO: 137 - DNA, 20 bp, synthetic construct
  source 1..20: mol_type = other DNA
gcagatactacgagactgat

SEQ ID NO: 138 - protein, 7 aa, synthetic construct
ADTTRLM

(19) United States
(12) Patent Application Publication
(10) Pub. No.: US 2026/0297604 A1
(43) Pub. Date: Oct. 1, 2026

(54) FRUIT WITH INCREASED FRUIT SIZE, ANTIOXIDANTS, AND RESISTANCE TO BLOSSOM END ROT

(71) Applicants: BOYCE THOMPSON INSTITUTE FOR PLANT RESEARCH, INC., Ithaca, NY (US); CORNELL UNIVERSITY, Ithaca, NY (US)

(72) Inventors: Carmen CATALA, Ithaca, NY (US); Philippe NICOLAS, Ithaca, NY (US); Richard PATTISON, Ithaca, NY (US)

(73) Assignees: BOYCE THOMPSON INSTITUTE FOR PLANT RESEARCH, INC., Ithaca, NY (US); CORNELL UNIVERSITY, Ithaca, NY (US)

(21) Appl. No.: 19/489,097

(22) PCT Filed: May 30, 2024
(86) PCT No.: PCT/US2024/031632

§ 371 Date: Dec. 1, 2025

Related U.S. Application Data
(60) Provisional application No. 63/504,949, filed on May 30, 2023.

(51) Int. Cl. C12N 15/82; A01H 1/00; A01H 1/04; C12N 9/12; C12N 15/10; C12N 15/11; C12N 15/113

(52) U.S. Cl. CPC C12N 15/8271; A01H 1/045; A01H 1/122; C12N 9/1241; C12N 15/102; C12N 15/111; C12N 15/113; C12N 15/8223; C12N 15/8243; C12N 15/8262; C12Y 207/07027

(57) ABSTRACT

The present disclosure is directed to methods and compositions for increasing resistance to blossom end rot in fruit, increasing fruit size, and increasing antioxidant content in fruits. Embodiments of the disclosure also include plant cells, plants, and fruits including a modified ADP-glucose pyrophosphorylase (AGPase). Also included are nucleic acid constructs, and methods of breeding for enhanced blossom end rot resistance.

### CROSS-REFERENCE TO RELATED APPLICATIONS

This application claims priority of U.S. Provisional application No. 63/504,949 filed May 30, 2023, the entire contents being incorporated herein by reference as though set forth in full.

### STATEMENT REGARDING FEDERALLY SPONSORED RESEARCH OR DEVELOPMENT

This invention was made with government support under grant number IOS1339287 awarded by the National Science Foundation, and grant number 2019-67013-29240 awarded by the U.S. Department of Agriculture. The government has certain rights in the invention.

### INCORPORATION-BY-REFERENCE OF MATERIAL SUBMITTED IN ELECTRONIC FORM

The Contents of the electronic sequence listing (BTCO-102-PCT.xml; Size: 413,534 bytes; and Date of Creation: May 30, 2024) is herein incorporated by reference in its entirety.

### FIELD

The present disclosure is directed to methods and compositions for increasing resistance to blossom end rot in fruit, increasing fruit size, and increasing antioxidant content in fruits.

### BACKGROUND

The regulation of carbohydrate availability is crucial in plant growth and development under both favorable and challenging conditions. Appropriate carbon partitioning between photosynthetically active source tissues, such as leaves, and sink tissues, such as fruits and roots, is required for optimal plant growth and reproductive development. Starch, the major carbohydrate store in plants, plays a key role in maintaining sugar homeostasis in a fluctuating environment (MacNeill et al., “Starch as a Source, Starch as a Sink: The Bifunctional Role of Starch in Carbon Allocation,” J Exp Bot 68:4433-4453 (2017)). In source organs fixed carbon is exported to sink tissues, mainly in the form of sucrose, or transformed into transitory starch in the plastids, a carbon reservoir that accumulates during the day and is degraded at night to provide carbon and energy when photosynthesis is nonfunctional (Stitt and Zeeman, “Starch Turnover: Pathways, Regulation and Role in Growth,” Curr Opin Plant Biol 15:282-292 (2012)). The metabolic activity of photosynthetic leaves and phloem unloading has an effect on sugar accumulation in sink tissues, where carbohydrate metabolism and accumulation are also tightly regulated (Osorio et al., “An Update on Source-to-Sink Carbon Partitioning in Tomato,” Front Plant Sci 5:516 (2014)) to coordinate photoassimilate delivery and consumption. Sucrose allocated to sink tissues can be metabolized for starch biosynthesis, which acts as a more or less stable carbohydrate storage, depending on the nature of the sink tissue (MacNeill et al., “Starch as a Source, Starch as a Sink: The Bifunctional Role of Starch in Carbon Allocation,” J Exp Bot 68:4433-4453 (2017)). In dedicated storage sinks (e.g., seeds and tubers), starch accumulates for long-term energy storage. However, in other heterotrophic organs, such as reproductive tissues, starch constitutes a temporary carbon storage that is rapidly remobilized during specific developmental stages (Hedhly et al., “Starch Turnover and Metabolism During Flower and Early Embryo Development,” Plant Physiol 172:2388-2402 (2016)). Although the pattern of accumulation can vary between fruit species, several fruit store starch early during fruit growth, and degrade it during ripening (Roch et al., “Fruit Salad in the Lab: Comparing Botanical Species to Help Deciphering Fruit Primary Metabolism,” Front Plant Sci 10:836 (2019)).

Tomato (Solanum lycopersicum) is an important fruit crop and a model system for fleshy fruit development. During tomato fruit development, the majority of photoassimilates are imported as sucrose from the leaves, therefore, sucrose import and metabolism are considered major factors contributing to fruit sink strength and metabolic composition (Fridman et al., “A Recombination Hotspot Delimits a Wild-Species Quantitative Trait Locus for Tomato Sugar Content to 484 bp Within an Invertase Gene,” PNAS 97:4718-4723 (2000); Zanor et al., “RNA Interference of LIN5 in Tomato Confirms its Role In Controlling Brix Content, Uncovers the Influence of Sugars on the Levels of Fruit Hormones, and Demonstrates the Importance of Sucrose Cleavage for Normal Fruit Development and Fertility,” Plant Physiol 150:1204-1218 (2009); Ruan et al., “Molecular Regulation of Seed and Fruit Set,” Trends Plant Sci 17:656-665 (2012); Beauvoit ct al., “Model-Assisted Analysis of Sugar Metabolism Throughout Tomato Fruit Development Reveals Enzyme and Carrier Properties in Relation to Vacuole Expansion,” Plant Cell 26:3224-3242 (2014)). Tomato fruit store carbon as transitory starch, which peaks during fruit growth (Schaffer and Petreikov, “Sucrose-to-Starch Metabolism in Tomato Fruit Undergoing Transient Starch Accumulation,” Plant Physiol 113:739-746 (1997)) and is mobilized during ripening. It has been proposed that this transient starch accumulation contributes to maintaining sink strength during fruit development, and to fuel ripening associated metabolism (Osorio et al., “An Update on Source-to-Sink Carbon Partitioning in Tomato,” Front Plant Sci 5:516 (2014); Quinet et al., “Tomato Fruit Development and Metabolism,” Front Plant Sci. 10:1554 (2019)). A positive correlation has been found between the size of the starch pool and the final levels of fruit soluble sugars (Baxter et al., “Fruit Carbohydrate Metabolism in an Introgression Line of Tomato with Increased Fruit Soluble Solids,” Plant Cell Physiol 46:425-437 (2005); Petreikov et al., “Carbohydrate Balance and Accumulation During Development of Near-Isogenic Tomato Lines Differing in the AGPase-LI Allele,” J Amer Soc Hort Sci 134:134-140 (2009); Powell et al., “Uniform Ripening Encodes a Golden 2-Like Transcription Factor Regulating Tomato Fruit Chloroplast Development,” Science 336:1711-1715 (2012); Sagar et al “SIARF4, An Auxin Response Factor Involved in the Control of Sugar Metabolism During Tomato Fruit Development,” Plant Physiol 161:1362-1374 (2013)), and between the activity levels of ADP-glucose pyrophosphorylase (AGPase), a limiting enzyme in starch synthesis (Geigenberger P., “Regulation of Starch Biosynthesis in Response to a Fluctuating Environment,” Plant Physiol 155:1566-1577 (2011)), and fruit sugar content at ripening (Centeno et al., “Malate Plays a Crucial Role in Starch Metabolism, Ripening, and Soluble Solid Content of Tomato Fruit and Affects Postharvest Softening,” Plant Cell 23:162-184 (2011); Hou et al., “Responses of Water Accumulation and Solute Metabolism in Tomato Fruit to Water Scarcity and Implications for Main Fruit Quality Variables,” J Exp Bot 71:1249-1264 (2020)). Thus, it has been suggested that degradation of the starch accumulated in the immature fruit contributes to an increase in sugar levels in the ripe fruit (Quinet et al., “Tomato Fruit Development and Metabolism,” Front Plant Sci. 10:1554 (2019)), which support the metabolic changes associated with ripening (Biais et al., “Remarkable Reproducibility of Enzyme Activity Profiles in Tomato Fruits Grown Under Contrasting Environments Provides a Roadmap for Studies of Fruit Metabolism,” Plant Physiol 164:1204-1221 (2014)). An additional proposed role for starch is to act as a buffer for carbon supply fluctuations during fruit set and growth under abiotic stress conditions (Ruan et al., “Molecular Regulation of Seed and Fruit Set,” Trends Plant Sci 17:656-665 (2012); Roch et al., “Fruit Salad in the Lab: Comparing Botanical Species to Help Deciphering Fruit Primary Metabolism,” Front Plant Sci 10:836 (2019); Yu et al., “Starch and Sugars as Determinants of Postharvest Shelf Life and Quality: Some New and Surprising Roles,” Curr Opin Biotechnol 78:102844 (2022)). However, the physiological significance of starch accumulation and metabolism during fruit development is not fully understood.

Starch metabolism is also critical in plant tolerance to abiotic stress, serving as a carbon reserve that is remobilized to release energy, sugars, and derived metabolites needed for stress mitigation (Pommerrenig et al., “In Concert: Orchestrated Changes in Carbohydrate Homeostasis are Critical for Plant Abiotic Stress Tolerance,” Plant Cell Physiol 59:1290-1299 (2018); Dong and Beckles, “Dynamic Changes in the Starch-Sugar Interconversion Within Plant Source and Sink Tissues Promote a Better Abiotic Stress Response,” J Plant Physiol 234-235:80-93 (2019)). Starch accumulation can be induced in certain organs and developmental stages in response to various abiotic stresses (Dong and Beckles, “Dynamic Changes in the Starch-Sugar Interconversion Within Plant Source and Sink Tissues Promote a Better Abiotic Stress Response,” J Plant Physiol 234-235:80-93 (2019)), and enzymes involved in starch metabolism are tightly regulated during environmental stress (Thalmann and Santelia, “Starch as a Determinant of Plant Fitness Under Abiotic Stress,” New Phytol 214:943-951 (2017); Fàbregas and Fernie, “The Metabolic Response to Drought,” J Exp Bot 70:1077-1085 (2019)). An increase in sugar supply, caused by activation of starch mobilization, may play an important role in withstanding the effects of abiotic stress. As highly hydroxylated soluble molecules, sugars can function as ROS scavengers, osmoprotectants, and protein and membrane stabilizers. In addition, sugars can act as signaling molecules, thus participating in stress perception, signaling, and gene expression regulation during abiotic stress responses (Saddhe et al., “Plant Sugars: Homeostasis and Transport Under Abiotic Stress in Plants. Physiol Plant 171:739-755 (2021)). Accordingly, a tight control of sugar homeostasis, through starch or sucrose metabolism, is required during the stress response to maintain or even increase sugar levels under challenging environmental conditions (Pommerrenig et al., “In Concert: Orchestrated Changes in Carbohydrate Homeostasis are Critical for Plant Abiotic Stress Tolerance,” Plant Cell Physiol 59:1290-1299 (2018); Saddhe et al., “Plant Sugars: Homeostasis and Transport Under Abiotic Stress in Plants. Physiol Plant 171:739-755 (2021)). Although starch-sugar interconversions have been suggested as a key determinant of plant fitness under abiotic stress (Thalmann and Santelia, “Starch as a Determinant of Plant Fitness Under Abiotic Stress,” New Phytol 214:943-951 (2017); Dong and Beckles, “Dynamic Changes in the Starch-Sugar Interconversion Within Plant Source and Sink Tissues Promote a Better Abiotic Stress Response,” J Plant Physiol 234-235:80-93 (2019)), most information about the contribution of starch turnover to the plant stress response has been obtained from Arabidopsis and cereal grains. Notably, the role of starch metabolism in the regulation of carbon use during fruit development under abiotic stress conditions, remains largely unexplored (Ma et al., “Molecular Genetic Analyses of Abiotic Stress Responses During Plant Reproductive Development,” J Exp Bot 71:2870-2885 (2020); Yu et al., “Starch and Sugars as Determinants of Postharvest Shelf Life and Quality: Some New and Surprising Roles,” Curr Opin Biotechnol 78:102844 (2022)).

Blossom end rot (BER) is a disease affecting tomato fruit as well as other fruits such as peppers, watermelons, and eggplants, resulting in significant yield losses worldwide. BER causes the presence of damaged and necrotic tissue at the distal end in fruit that typically first appears in the placenta, and later affects the pericarp. BER is induced by environmental stress, such as drought, although it is also thought that calcium deficiency plays a role in BER incidence (Topcu et al., “Blossom-End Rot: A Century-Old Problem in Tomato (Solanum lycopersicum L.) and Other Vegetables,” Mol Hort 2:1 (2022)). Due to these factors, BER is difficult to manage in diverse agricultural settings. Effective solutions to reduce or prevent the incidence of BER and impart BER resistance to fruit are needed.

This disclosure is directed to overcoming these and other deficiencies in the art.

### SUMMARY

One aspect of the present disclosure relates to a method for imparting blossom end rot resistance to fruit from a plant. The method comprises modifying a plant or a plant cell to reduce or eliminate the expression, stability, and/or activity of ADP-glucose pyrophosphorylase (AGPase) protein, where the reduction or elimination of the expression, stability, and/or activity of AGPase protein is effective in reducing or eliminating starch synthesis and imparting blossom end rot resistance to fruit from the plant or a plant produced from the plant cell, as compared to fruit from a plant without the modification.

Another aspect of the present disclosure relates to a method of increasing size of fruit from a plant. The method comprises modifying a plant or a plant cell to reduce or eliminate the expression, stability, and/or activity of ADP-glucose pyrophosphorylase (AGPase) protein, where the reduction or elimination of the expression, stability, and/or activity of AGPase protein is effective in reducing or eliminating starch synthesis and increasing fruit size in fruit from the plant or a plant produced from the plant cell, as compared to fruit from a plant without the modification.

A further aspect of the present disclosure relates to a method of increasing antioxidants in fruit from a plant. The method comprises modifying a plant or a plant cell to reduce or eliminate the expression, stability, and/or activity of ADP-glucose pyrophosphorylase (AGPase) protein, where the reduction or elimination of the expression, stability, and/or activity of AGPase protein is effective in reducing or eliminating starch synthesis and increasing antioxidant levels in a fruit from the plant or a plant produced from the plant cell, as compared to a fruit from a plant without the modification.

Another aspect of the present disclosure relates to a plant cell comprising a nucleic acid molecule comprising a modified ADP-glucose pyrophosphorylase (AGPase) gene encoding an AGPase protein or fragment thereof, where the expression, stability, and/or activity of the AGPase protein is reduced or eliminated in the plant cell as compared to a plant cell without the modification.

Yet another aspect of the present disclosure relates to a plant cell comprising an inhibitory polynucleotide targeting an AGPase gene encoding an AGPase protein, wherein the expression, stability, and/or activity of the AGPase protein is reduced or eliminated in the plant cell as compared to a plant cell without the inhibitory polynucleotide.

A further aspect of the present disclosure relates to a nucleic acid construct comprising a nucleotide sequence targeting an AGPase gene comprising: (i) a guide RNA, or (ii) an inhibitory polynucleotide, a 5′ heterologous DNA promoter sequence, and a 3′ terminator sequence.

Another aspect of the present disclosure relates to a method of breeding for enhanced blossom end rot resistance. The method comprises providing a candidate plant, analyzing the candidate plant for the presence, in its genome, of a modified ADP-glucose pyrophosphorylase (AGPasc) polynucleotide encoding an AGPasc protein, wherein the expression, stability, and/or activity of said AGPase protein is reduced or eliminated as compared to a plant without the modification, identifying, based on said analyzing, a candidate plant suitable for breeding that includes in its genome, the modified ADP-glucose pyrophosphorylase (AGPase) polynucleotide, and breeding the identified plant with at least one other plant.

Yet another aspect of the present disclosure relates to a method for imparting blossom end rot resistance to fruit from a plant. The method comprises modifying a plant or a plant cell to reduce or eliminate the expression, stability, and/or activity of ADP-glucose pyrophosphorylase (AGPase) protein, where the reduction or elimination of the expression, stability, and/or activity of AGPase protein is effective in reducing or eliminating starch synthesis and imparting blossom end rot resistance to fruit from the plant or a plant produced from the plant cell, as compared to fruit from a plant without the modification, and selectively expressing a functional AGPase protein in non-fruit tissues.

As disclosed herein, the agravitropic tomato mutant adpressa (adp) was found to carry a mutation in the gene encoding the small subunit of ADP-glucose pyrophosphorylase (AGPase), which abolished starch synthesis. The effects of the lack of starch accumulation on the fruit were evaluated. Fruit development was largely unaffected, although adp fruit showed an increased size and a slight delay in ripening. It was surprisingly shown that the starch deficiency correlated with a remarkable resistance of the fruit to blossom-end-rot (BER), an environmental stress-induced physiological disorder. To assess the broader implications of the starch deficiency, changes in the adp fruit transcriptome and metabolome were analyzed and molecular pathways affected by the altered sugar metabolism were identified. These results suggested that disabling starch biosynthesis caused an important transcriptional reprograming and triggered metabolic shifts affecting central carbon metabolic pathways and lipid metabolism. The data also indicated that the molecular and metabolic changes in adp were potentially mediated by epigenetic regulators and the activation of sugar signaling, leading to enhanced growth and stress response pathways. These results provided insights into the role of carbohydrate metabolism in regulating fruit development and stress responses, with broad relevance for the development of strategies to enhance fruit stress tolerance.

### BRIEF DESCRIPTION OF THE DRAWINGS

FIGS. 1A-1D show the characterization of the tomato mutant adpressa (adp). FIG. 1A is a photograph of wild type (WT) and adp six-week-old plants grown in greenhouse conditions showing the decumbent stems of adp. Scale bar=10 cm. FIG. 1B is a photograph of root agravitropism of seedlings grown in agar plates. Scale bar=1 cm. FIG. 1C is an illustration of map-based cloning of adp. The adp mutation mapped to an 86 kb region of chromosome 7 that contained 11 gene models including Solanum lycopersicum APSI (SEQ ID NO:1) shown with the single base pair deletion in red (SEQ ID NO:130). A protein diagram shows the characteristic motifs and domains labeled, the frame shift (white X) and the premature stop codon (asterisk). FIG. 1D is a photograph of Lugol staining of starch in wild type and adp vegetative tissues. Starch is revealed by dark purple-blue color in root (left), stem (middle) and leaves (right). Arrows show the starch granules as aggregated spots at the root tip, and at the innermost layer of the cortex in stems. Scale bars: root=150 μm; stem=1 mm; leaves=10 mm.

FIGS. 2A-2B show starch content in wild type and adp throughout fruit development. FIG. 2A is a photograph of Lugol staining of fruit cross sections revealing starch as a dark blue color. FIG. 2B is a graph of starch quantification in pericarp and placenta dissected from WT, adp and APS1pro::APS1 (CL) fruit at four developmental stages (dpa (days post anthesis), MG (mature green)). Statistically significant differences were determined using Student's t-test: ***=p-value <0.0001, n=12. Scale bar=10 mm.

FIGS. 3A-3F show phenotypic characteristics of WT, adp and CL fruit. FIG. 3A is a representative photograph of fruit, FIG. 3B is a graph of fruit weight and FIG. 3C is a graph of fruit volume of WT, adp and CL (dpa (days post anthesis), MG (mature green)). Statistically significant differences were determined using Student's t-test: *=p-value <0.05, **=p-value <0.001, n=70. FIG. 3D is a graph of time to the breaker stage in WT, adp and CL fruit. Statistically significant differences were determined using Student's t-test: *=p-value <0.05, n=50. FIG. 3E is a photograph of representative pictures showing BER symptoms (white arrow) in fruit grown under water stress. FIG. 3F is a graph showing BER incidence in WT, adp and CL fruit under control and water stress conditions. BER incidence was calculated as (number of tagged fruit with BER symptoms/total number of tagged fruit per plant)×100. Data are means (+SD) of three independent experiments (n>150). Each experiment was performed using 5 plants per genotype and at least 10 fruit per plant. None of the adp fruit were affected by BER. Scale bar=1 cm.

FIGS. 4A-4F are graphs, diagrams, and heat maps of transcriptome analysis of adp fruit. FIG. 4A is a graph of the number of differentially expressed genes (“DEGs”) in placenta and pericarp and FIG. 4B is a Venn diagram showing DEGs shared between 6 dpa, 20 dpa, mature green (MG) and ripe fruit. UP, upregulated; DOWN, downregulated. FIG. 4C is a heatmap showing the expression profiles of five DEGs shared between different fruit stages in placenta and pericarp. Columns from left to right on each arrow: 6 dpa, 20 dpa, MG, and ripe. FIGS. 4D and 4E are graphs of selected gene ontology (“GO”) terms enriched in up- and downregulated genes at different developmental stages. Columns from left to right on each arrow: 6 dpa, 20 dpa, MG, and ripe. Point size represents the number of DEGs (Count) in each GO term. All GO terms significantly enriched (adjusted p<0.05) in each tissue and developmental stage are listed in Table 4. FIG. 4F is a heatmap showing examples of gene expression changes related to TOR signaling, cell cycle related pathways, DNA repair, and epigenetic and stress responses, in the placenta of adp fruit at 6 dpa, 20 dpa and MG stage.

FIG. 5 shows box plots of changes in levels of sugars and amino acids in adp, WT and CL placenta throughout fruit development. Box plots show the distribution dynamics of a subset of the metabolites identified by GC/MS with significant changes (FDR<0.05) in both, adp vs. WT and adp vs. CL, comparisons. MG, mature green. Statistically significant differences were determined using Student's t-test (*=p-value <0.05). Data can be found in Table 6.

FIG. 6 is a heatmap of metabolomic analysis of the placenta of adp, WT and CL fruit at 20 dpa. The heatmap shows metabolites identified by untargeted LC-MS/MS with significant changes (FDR<0.05) in both adp vs. WT and adp vs. CL pairwise comparisons. Only metabolites with best match score >80% to an in-house spectral library and/or mzcloud references are shown. Data can be found in Table 7. Individual columns represent biological replicates of WT, CL and adp. Fold changes are indicated in blue (decreased level) and red (increased level).

FIGS. 7A-7C show lipid related gene expression and metabolite changes in adp fruit. FIG. 7A is a heatmap showing expression profiles of lipid related DEGs in placenta and pericarp at different developmental stages. Columns from left to right on each arrow for each genotype: 6 dpa, 20 dpa, MG, and ripe. FIG. 7B is a lipidomic analysis of the placenta of adp, WT and CL fruit at 20 dpa. The heatmap shows lipids with significant changes (FDR<0.05) in both, adp vs. WT and adp vs. CL comparisons. Data can be found in Table 8. Sample replicates are labeled 1-4. FIG. 7C shows photographs of lipid droplet accumulation in the septum of 20 dpa adp fruit, visualized by staining with BODIPY (green). Top: fluorescence only (505 nm excitation; 515 nm emission). Bottom: merged bright light and florescence images. Scale bar=260 μm.

FIG. 8 is an illustration of an overview of metabolic changes in the placenta of growing adp fruit compared to WT and CL. Metabolites with reduced levels, or genes downregulated in adp are shown in blue. Metabolites with increased levels, or genes upregulated in adp are shown in red. Metabolites not measured or without significant changes are shown in black. Red arrows show the pathway to lipid biosynthesis predicted to be enhanced in adp. ADCS, Aminodeoxychorismate synthase; DGK, Diacylglycerol kinase; ELO, Elongation of fatty acids protein; GAPDH, Glyceraldehyde-3-phosphate dehydrogenase; GPAT, Glycerol-3-phosphate acyltransferase; HMG, 3-Hydroxy-3-methylglutaryl; IPCS1, Phosphatidylinositol: ceramide inositolphosphotransferase; LACS, Long-chain acyl-CoA synthetase; LCB2, LONG-CHAIN BASE2 subunit of serine palmitoyltransferase; LKR, Lysine-ketoglutarate reductase; PA, Phoshatidic acid; PI, Phosphatidyl inositol; PIP, phosphatidylinositol-4-phosphate.

FIGS. 9A-9B show photographs of Lugol staining of WT, adp, and CL transgenic lines complemented with the SIAPSI genomic clone: leaves (FIG. 9A) and fruit (FIG. 9B). Scale bar=10 mm.

FIG. 10 shows photographs of the development of BER symptoms during fruit growth. For each genotype the top panel shows a distal end view of the fruit and the bottom panel shows a distal cross-section. Scale bar=1 cm.

FIGS. 11A-11B shows the global gene expression differences between adp, WT and CL fruit tissues. Principal component (FIG. 11A) and hierarchical clustering (FIG. 11B) analyses of gene expression in placenta and pericarp from 6 dpa, 20 dpa, mature green (MG) and ripe fruit are shown. Sample replicates are shown as identically colored circles (FIG. 11A) or identical font color (FIG. 11B).

FIG. 12 shows graphs of the expression of cell wall and ethylene-related genes in the pericarp of adp, WT and CL fruit at the MG stage. Expression data (RPKM) are the mean (±SD) of three biological replicates. Statistically significant differences were determined using Student's t-test: **=p-value <0.001, n=3. ACS2: Aminocyclopropane-1-carboxylate synthase (ACC) 2; ACS4: ACC synthase 4; ACO6: 1-aminocyclopropane-1-carboxylate oxidase 6; ETR4: ethylene receptor 4; EXP1: Expansin 1; PM3: Pectin methylesterase 3; XTH5: Xyloglucan endotransglucosylase-hydrolase 5; PG: Polygalacturonase.

FIG. 13 shows graphs of changes in levels of soluble sugars throughout fruit development in adp, WT and CL fruit. MG: mature green. Data are means (+SD) of three independent experiments, with four biological replicates per experiment. Statistically significant differences were determined using Student's t-test: *=p-value <0.05, **=p-value <0.001, n=12.

FIGS. 14A-14C show graphs of clustering of the metabolite profiles of placenta (Pl) and pericarp (Pe) from adp, WT and CL fruit at different developmental stages. Principal component (FIGS. 14A and 14B), and hierarchical clustering (FIG. 14C) analysis of metabolites identified by GC/MS. Sample replicates are shown as identically colored circles in (FIG. 14A) and (FIG. 14B) and labeled 1 to 4 in (FIG. 14C). MG, mature green. RR, ripe.

FIGS. 15A-15D show graphs of principal component analysis of metabolites of adp, WT and CL placenta tissue from 20 dpa fruit. Metabolites were identified by untargeted LC-MS/MS using: C18-NEG (FIG. 15A), C18-POS (FIG. 15B), HILIC-NEG (FIG. 15C) and HILIC-POS (FIG. 15D). Negative mode (NEG) and positive mode (POS). Sample replicates are shown as identically colored circles.

FIGS. 16A-16F show graphs of clustering of the lipidomic profiles of placenta and pericarp from adp, WT and CL fruit at different developmental stages. Principal component analysis (FIGS. 16A-16D) and hierarchical clustering analysis (FIGS. 16E-16F) of compounds identified using an untargeted LC/MS analysis with both negative (NEG) and positive (POS) modes. Sample replicates are shown as identically colored circles (FIGS. 16A-16D) or numbered 1 to 4 (FIGS. 16E-16F). MG, mature green. RR, ripe.

FIGS. 17A-17B show photographs of lipid droplet accumulation in adp fruit placenta at 20 dpa visualized by staining with BODIPY (green). In both (FIG. 17A) and (FIG. 17B) the left panels show fluorescence only (505 nm excitation; 515 nm emission) and the right panels show merged bright light and florescence images. (FIG. 17B) shows a higher magnification of the area enclosed by a black rectangle in (FIG. 17A). Scale bar=670 μm in (FIG. 17A), 260 μm in (FIG. 17B).

FIGS. 18A-18D show the characterization of the tomato aps1 CRISPR mutant aps1-CR13 and phenotypic comparison with aps1-adp. (FIG. 18A) APS1 gene structure indicating the position of the aps1-adp and aps1-CR13 mutations. Red dashed lines indicate base pair deletions. Change in amino acids are also shown in red. WT, wild type. (FIG. 18B) APS1 protein diagram showing the characteristic motifs and domains labeled, the frame shift (white X) and the premature stop codon (asterisk) in the two aps1 mutants (aps1-adp and aps1-CR13). (FIG. 18C) 6-week-old wild-type (WT) plants and aps1 mutants grown in greenhouse conditions showing the decumbent stems of aps1-adp (Ailsa Craig, AC, background) and aps1-CR13 (M82 background). (FIG. 18D) Lugol staining of starch in wild-type and aps1 mutant leaf tissues. Starch accumulation is revealed by dark purple-blue color.

FIG. 19A-19E shows expression levels of promoter of tomato genes STM3 (Solyc01g092950) (FIG. 1A), SIMBP24 (Solyc01g105800) (FIG. 19B), and βCA1 (Solyc02g086820) (FIG. 19D). Also shown are construct maps of promoters of tomato gene SIMBP24 (Solyc01g105800) (FIG. 19C) and tomato gene βCA1 (Solyc02g086820) (FIG. 19E) assembled with APS1 into a pGWB401 backbone.

FIG. 20A-20C shows the expression levels of melon promoters MELO3C022515 (FIG. 20A), MELO3C007716 (FIG. 20B), and MELO3C009958 (FIG. 20C) in different plant tissues.

### DETAILED DESCRIPTION

Embodiments of the disclosure relate to methods of imparting blossom end rot resistance, increasing size, and increasing antioxidant content of fruit, and compositions useful in imparting blossom end rot resistance, increasing size, and increasing antioxidant content of fruit are also provided.

Unless otherwise indicated, the definitions and embodiments described in this, and other sections are intended to be applicable to all embodiments and aspects of the present application herein described for which they are suitable as would be understood by a person skilled in the art.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure. In another example, reference to “a compound” includes both a single compound and a plurality of different compounds.

The term “about” includes being within a statistically meaningful range of a value. Such a range can be within an order of magnitude, such as within 10% or within 5% of a given value or range.

The term “and/or” as used herein means that the listed items are present, or used, individually or in combination. In effect, this term means that “at least one of” or “one or more” of the listed items is used or present.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “involving”, “having”, and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps.

A “nucleic acid”, “nucleotide”, or “polynucleotide” is a polymeric compound comprised of covalently linked subunits called nucleotides. Nucleic acids include polyribonucleic acid (“RNA”) and polydeoxyribonucleic acid (“DNA”), both of which may be single-stranded or double-stranded. DNA includes, but is not limited to, cDNA, genomic DNA, plasmid DNA, synthetic DNA, and semi-synthetic DNA. DNA may be linear, circular, or supercoiled.

A “gene” refers to an assembly of nucleotides that encode a functional RNA or a polypeptide and includes cDNA and genomic DNA nucleic acids. “Gene” also refers to a nucleic acid fragment that expresses a specific protein or polypeptide, optionally including regulatory sequences preceding (5′ noncoding sequences) and following (3′ noncoding sequences) the coding sequence. “Endogenous gene” refers to a native gene in its natural location in the genome of an organism. A “foreign” gene, “heterologous” gene, or “exogenous” gene refers to a gene not normally found in the host organism, but that is introduced into the host organism by gene transfer. Heterologous genes can comprise native genes inserted into a non-native organism, or chimeric genes, such as a native gene under control of a different promoter than its endogenous promoter. A “transgene” is a gene that has been introduced into the genome by a transformation or transfection procedure. A “foreign” gene or “exogenous” gene refers to a gene not normally found in the host organism, but that is introduced into the host organism by gene transfer. Foreign genes can comprise native genes inserted into a nonnative location, or organisms, as well as chimeric genes.

A “coding region” is a portion of a nucleic acid, which is transcribed and translated into a polypeptide or protein.

A “polypeptide” is a naturally occurring or synthetic peptide, oligopeptide, polypeptide, gene product, expression product, or protein comprising an amino acid sequence, where the amino acids are joined to each other by peptide bonds or modified peptide bonds.

The term “fragment” when referring to a polynucleotide or polypeptide will be understood to mean a nucleotide or polypeptide sequence of reduced length relative to the reference nucleic acid or polypeptide sequence (e.g., a wild type) and comprising, over the common portion, a nucleotide or polypeptide sequence identical to the reference sequence. Such a fragment according to the present disclosure may be, where appropriate, included in a larger polynucleotide or polypeptide of which it is a constituent. Such fragments comprise, or alternatively consist of, oligonucleotides or polypeptides ranging in length from at least 8, 10, 12, 15, 18, 20 to 25, 30, 40, 50, 70, 80, 100, 200, 500, 1000, 1500, or any number or range therein, consecutive nucleotides of a nucleic acid or amino acids of a polypeptide described herein.

A “reference sequence” means a nucleic acid or amino acid sequence used as a comparator for another nucleic acid or amino acid sequence, respectively, when determining sequence identity. A reference sequence can be a wildtype sequence or a sequence that has not been modified as described herein.

“Sequence identity,” “percent identity,” or “% identical” refers to the exactness of a match between a reference sequence and a sequence being compared to it when optimally aligned. For example, sequence alignments and percent identity calculations may be determined using a variety of comparison methods designed to detect homologous sequences including, but not limited to, the Multalin program (Corpet, “Multiple Sequence Alignment with Hierarchical Clustering,” Nucleic Acids Res. 16:10881-90 (1988), which is hereby incorporated by reference in its entirety) or the Megalign® program of the LASERGENE® bioinformatics computing suite (DNASTAR® Inc., Madison, Wis.). Sequences may also be aligned using algorithms known in the art including, but not limited to, CLUSTAL V algorithm or the BLASTN or BLAST 2 sequence programs.

As used herein, the term “plant cell” includes cells, protoplasts, cell tissue cultures from which plants can be regenerated, calli, clumps, and cells that are intact in plants or parts of plants (plant parts) including, but not limited to seeds, leaves, stems, roots, vegetative buds, floral buds, meristems, embryos, hypocotyls, cotyledons, endosperm, sepals, petals, pistils, carpels, stamens, anthers, microspores, pollen, pollen tubes, ovules, nucellar tissue, ovaries, and other plant tissue or cells. In some embodiments, the plant cell is a protoplast.

Preferences and options for a given aspect, feature, embodiment, or parameter of the disclosure should, unless the context indicates otherwise, be regarded as having been disclosed in combination with any and all preferences and options for all other aspects, features, embodiments, and parameters of the disclosure.

### ADP-Glucose Pyrophosphorylase Genes, Proteins, and Subunits

One aspect of the present disclosure relates to a method for imparting blossom end rot resistance to fruit from a plant. The method comprises modifying a plant or a plant cell to reduce or eliminate the expression, stability, and/or activity of an ADP-glucose pyrophosphorylase (AGPase) protein, where the reduction or elimination of the expression, stability, and/or activity of AGPase protein is effective in reducing or eliminating starch synthesis and imparting blossom end rot resistance to fruit from the plant or a plant produced from the plant cell, as compared to fruit from a plant without the modification.

This aspect of the present disclosure can be carried out with any of the embodiments disclosed herein.

In some embodiments, methods of the present disclosure comprise modifying a plant or a plant cell to reduce or eliminate the expression, stability, and/or activity of an ADP-glucose pyrophosphorylase (“AGPase”) protein. AGPase is enzyme that catalyzes the first committed step in starch synthesis. AGPase synthesizes ADP-glucose from Glucose-1 phosphate and ATP. AGPase typically functions as a heterotetramer of two AGPase small subunits (“APS”) and two AGPase large subunits (“APL”). Accordingly, some embodiments of the disclosure involve modifying a plant or a plant cell to reduce or eliminate the expression, stability, and/or activity of one or more APS subunits and/or one or more APL subunits as described infra relative to an unmodified, or wild-type APS subunit and/or APL subunit.

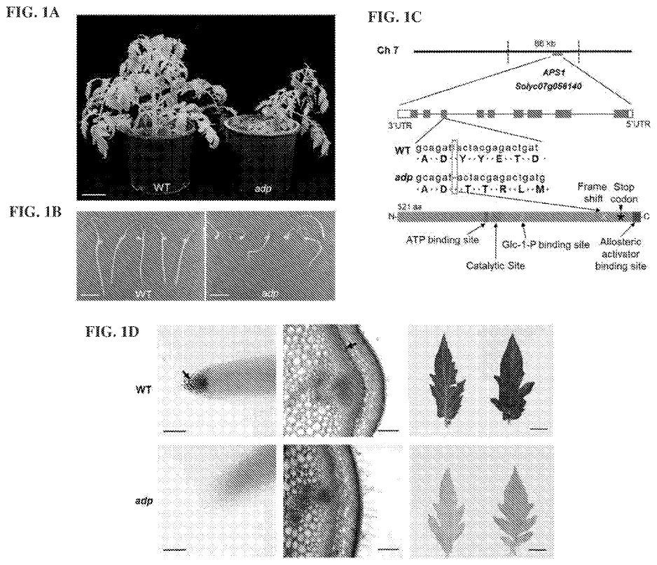

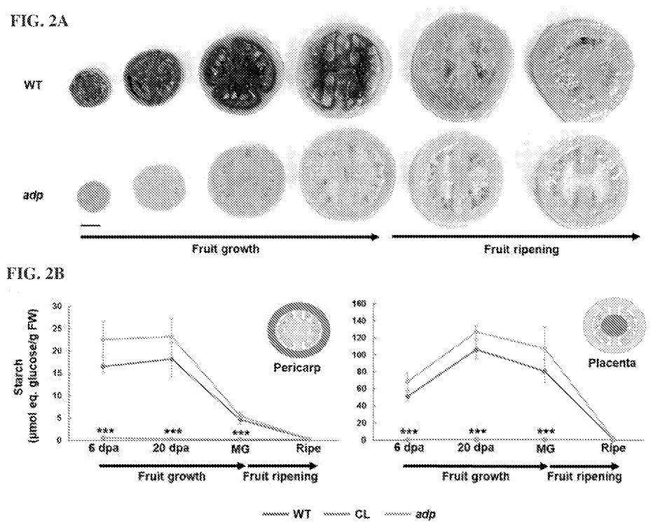

Starch content in various tissues in a plant can be detected by any means known in the art. For example, starch can be detected by staining with Lugol's solution as shown in FIGS. 1D, FIG. 2A, and FIGS. 9A-B. Starch can also be quantified as shown in FIG. 2B by digestion with α-amylase and amyloglucosidase as described in Vargas-Ortiz et al., “Grain Amaranths are Defoliation Tolerant Crop Species Capable of Utilizing Stem and Root Carbohydrate Reserves to Sustain Vegetative and Reproductive Growth After Leaf Loss,” PLOS One 8: e67879 (2013), which is hereby incorporated by reference in its entirety.

In some embodiments of the disclosure, the AGPase polypeptide or protein (or fragment or variant thereof) is an ADP-glucose pyrophosphorylase small subunit (APS) protein and/or an ADP-glucose pyrophosphorylase large subunit (APL) protein. Accordingly, the polynucleotide or gene (or fragment or variant thereof) in some embodiments of the disclosure is an ADP-glucose pyrophosphorylase small subunit (APS) gene and/or an ADP-glucose pyrophosphorylase large subunit (APL) gene. In some embodiments, the AGPase gene comprises an ADP-glucose pyrophosphorylase small subunit (APS) coding sequence or an ADP-glucose pyrophosphorylase large subunit (APL) coding sequence.

A plant may comprise one or more than one endogenous APS and/or APL subunit gene(s). The expression of these various subunit genes may vary by level, tissue type, developmental timing, as well as in response to various environmental factors, as non-limiting examples. For example, tomato (Solanum lycopersicum) includes three APS subunit genes. Tomato APS1 is expressed in all tissue types, while tomato APS2 is only expressed in the first roots, and APS3 is only expressed in the stamen. In some embodiments, the ADP-glucose pyrophosphorylase protein whose expression, stability, and/or activity is reduced or eliminated is the expression product of an APS or APL gene that is expressed in the leaves and/or developing fruit of the plant.

Endogenous ADP-glucose pyrophosphorylase small subunit (APS) and large subunit (APL) genes, coding sequences, and amino acid sequences from various plant species are disclosed herein as SEQ ID NOs: 1-129 (see Tables 1 and 13, infra). These endogenous (which may also be referred to as unmodified, wild-type, target, or reference) sequences are used in various embodiments of the disclosure to generate modified ADP-glucose pyrophosphorylasc small subunits and/or large subunits that reduce or eliminate the expression, stability, and/or activity of AGPase.

In some embodiments, the APS gene is an APS1 gene from Solanum lycopersicum (i.e., Solgenomics.net Accession No. Solyc07g056140, which is hereby incorporated by reference in its entirety). The genomic sequence (SEQ ID NO:1), coding sequence (SEQ ID NO: 2), and amino acid sequence (SEQ ID NO:3) of Solanum lycopersicum APS1 are provided in Table 13 infra. In some embodiments, the APS gene is an APS2 gene from Solanum lycopersicum (i.e., Solgenomics.net Accession No. Solyc08g015670, which is hereby incorporated by reference in its entirety). The genomic sequence (SEQ ID NO:4), coding sequence (SEQ ID NO:5), and amino acid sequence (SEQ ID NO:6) of Solanum lycopersicum APS2 are provided in Table 13 infra. In some embodiments, the APS gene is an APS3 gene from Solanum lycopersicum (i.e., Solgenomics.net Accession No. Solyc12g011120, which is hereby incorporated by reference in its entirety). The genomic sequence (SEQ ID NO:7), coding sequence (SEQ ID NO:8), and amino acid sequence (SEQ ID NO:9) of Solanum lycopersicum APS3 are provided in Table 13 infra.

Additional exemplary endogenous APS sequences from Solanum melogena, Capsicum annuum, Cucumis sativus, Cucurbita maxima, Cucurbita pepo, Cucumis melo, Cucurbita moschata, and Citrullus lanatus are described in Tables 1 and 13 infra and are intended to illustrate the various types of AGPase APS sequences that are useful in embodiments of the present disclosure.

Accordingly, in some embodiments, the APS gene encoding an AGPase protein of embodiments of the present disclosure comprises the nucleotide sequence encoding the amino acid sequence of any one of SEQ ID NOs: 3, 6, 9, 21, 24, 36, 39, 42, 54, 66, 69, 81, 93, 105, 117, and 120, or having at least 90% sequence identity to the amino acid sequence of any one of SEQ ID NOs: 3, 6, 9, 21, 24, 36, 39, 42, 54, 66, 69, 81, 93, 105, 117, and 120. Due to genetic variation that can occur between various species and/or varieties of the plants described herein, the APS gene encoding an AGPase protein may comprise at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the genomic, coding, or amino acid sequence of any one of SEQ ID NOs: 1-9, 19-24, 34-42, 52-54, 64-69, 79-81, 91-93, 103-105, and 115-120.

In some embodiments, the APL gene is an APLI gene from Solanum lycopersicum (i.e., Solgenomics.net Accession No. Solyc01g079790, which is hereby incorporated by reference in its entirety). The genomic sequence (SEQ ID NO:10), coding sequence (SEQ ID NO:11), and amino acid sequence (SEQ ID NO:12) of Solanum lycopersicum APLI are provided in Table 13 infra. In some embodiments, the APL gene is an APL2 gene from Solanum lycopersicum (i.e., Solgenomics.net Accession No. Solyc07g019440, which is hereby incorporated by reference in its entirety). The genomic sequence (SEQ ID NO:13), coding sequence (SEQ ID NO:14), and amino acid sequence (SEQ ID NO:15) of Solanum lycopersicum APL2 are provided in Table 13 infra. In some embodiments, the APL gene is an APL3 gene from Solanum lycopersicum (i.e., Solgenomics.net Accession No. Solyc01g109790, which is hereby incorporated by reference in its entirety). The genomic sequence (SEQ ID NO:16), coding sequence (SEQ ID NO:17), and amino acid sequence (SEQ ID NO:18) of Solanum lycopersicum APL3 are provided in Table 13 infra.

Additional exemplary endogenous APL sequences from Solanum melogena, Capsicum annuum, Cucumis sativus, Cucurbita maxima, Cucurbita pepo, Cucumis melo, Cucurbita moschata, and Citrullus lanatus are described in Tables 1 and 13 infra and are intended to illustrate the various types of AGPase APL sequences that are useful in the embodiments of the present disclosure.

In some embodiments, the APL gene comprises the nucleotide sequence encoding the protein sequence of any one of SEQ ID NOs: 12, 15, 18, 27, 30, 33, 45, 48, 51, 57, 60, 63, 72, 75, 78, 84, 87, 90, 96, 99, 102, 108, 111, 114, 123, 126, and 129 or having at least 90% sequence identity to the amino acid sequence of any one of SEQ ID NOs: 12, 15, 18, 27, 30, 33, 45, 48, 51, 57, 60, 63, 72, 75, 78, 84, 87, 90, 96, 99, 102, 108, 111, 114, 123, 126, and 129. Due to genetic variation that can occur between various species and/or varieties of the plants described herein, the APL gene encoding an AGPase protein may comprise at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the genomic, coding, or amino acid sequence of any one of SEQ ID Nos: 10-18, 25-33, 43-51, 55-63, 70-78, 82-90, 94-102, 106-114, and 121-129.

TABLE 1
Exemplary Endogenous AGPase Sequences

| Crop | AGPase Subunits | SEQ ID NOs: |
|---|---|---|
| Tomato | APS1, APS2, APS3 | 1-3, 4-6, 7-9 |
| (Solanum lycopersicum) | APL1, APL2, APL3 | 10-12, 13-15, 16-18 |
| Eggplant | APS1, APS2 | 19-21, 22-24 |
| (Solanum melongena) | APL1, APL2, APL3 | 25-27, 28-30, 31-33 |
| Pepper | APS1, APS2, APS3 | 34-36, 37-39, 40-42 |
| (Capsicum annuum) | APL1, APL2, APL3 | 43-45, 46-48, 49-51 |
| Cucumber | APS1 | 52-54 |
| (Cucumis sativus) | APL1, APL2, APL3 | 55-57, 58-60, 61-63 |
| Squash | APS1, APS3 | 64-66, 67-69 |
| (Cucurbita maxima) | APL1, APL2, APL3 | 70-72, 73-75, 76-78 |
| Zucchini | APS1 | 79-81 |
| (Cucurbita pepo) | APL1, APL2, APL3 | 82-84, 85-87, 88-90 |
| Melon | APS1 | 91-93 |
| (Cucumis melo) | APL1, APL2, APL3 | 94-96, 97-99, 100-102 |
| Watermelon | APS1 | 103-105 |
| (Citrullus lanatus) | APL1, APL2, APL3 | 106-108, 109-111, |
| | | 112-114 |
| Pumpkin | APS1, APS3 | 115-117, 118-120 |
| (Cucurbita moschata) | APL1, APL2, APL3 | 121-123, 124-126, |
| | | 127-129 |

### Modifications of AGPase: Mutagenesis & Genome Editing

In some embodiments of the disclosure, modifying a plant or plant cell to reduce or eliminate the expression, stability, and/or activity of an ADP-glucose pyrophosphorylase (AGPase) protein comprises: (i) introducing a mutation into a coding sequence and/or a regulatory sequence of an AGPase gene encoding the AGPase protein; or (ii) introducing into the plant or plant cell an inhibitory polynucleotide targeting the AGPase gene encoding the AGPase protein. As used herein, a “regulatory sequence” is a sequence that regulates the expression and/or activity of a gene or protein.

In some embodiments of the disclosure, the expression stability, and/or activity of an AGPase can be reduced or eliminated by mutating one or more APS and/or APL gencs. In some embodiments, the one or more APS and/or APL genes in a plant or plant cell has a mutation that inhibits normal expression, stability, and/or activity of the gene or the protein encoded by the gene.

The terms “mutation” or “genome edit” mean a human-induced change in the genetic sequence compared to a wild type or reference sequence (i.e., a sequence that exists in nature). In some embodiments, mutations are those that cause the gene coding sequence or a promoter to not be expressed or to have a reduced level of mRNA expression, or those that reduce the activity, expression, or stability of the encoded protein. In some embodiments, the methods of the present disclosure comprise introducing the mutation by mutagenesis or genome editing. The terms “modify”, “modifying,” and the like used herein encompass modifying by, e.g., mutation or genome editing.

A mutation may be, without limitation, a “substitution mutation”, which is a change in the nucleic acid sequence that results in the substitution of an amino acid for another amino acid; a “nonsense mutation” or “stop codon mutation”, which is a change in the nucleic acid sequence that results in the introduction of a premature stop codon and thus the termination of translation (resulting in a truncated protein); an “insertion mutation” of one or more amino acids, due to one or more codons having been added in the coding sequence of the nucleic acid; a “deletion mutation” of one or more amino acids, due to one or more codons having been deleted in the coding sequence of the nucleic acid; and a “frameshift mutation”, resulting in the nucleic acid sequence being translated in a different frame downstream of the mutation, or any combination of mutation types. A frameshift mutation can have various causes, such as the insertion, deletion, substitution, and/or duplication of one or more nucleotides. Mutations that affect pre-mRNA splicing (splice junction mutations) can also result in frameshifts. A “splice junction mutation”, which alters or abolishes the correct splicing of the pre-mRNA sequence, resulting in a protein of different amino acid sequence than the wild type. For example, one or more exons may be skipped during RNA splicing, resulting in a protein lacking the amino acids encoded by the skipped exons. Alternatively, the reading frame may be altered through incorrect splicing, or one or more introns may be retained, or alternate splice donors or acceptors may be generated, or splicing may be initiated at an alternate position (e.g., within an intron), or alternate polyadenylation signals may be generated. Accordingly, in some embodiments, the mutation is an insertion, a deletion, a substitution mutation, or any combination thereof. In some embodiments, the mutation is a “knock-out” mutation.

As used herein, a “knock-out” mutation is a mutant allele having no functional AGPase expression, i.e., the mutant allele produces no AGPase protein having the ability to synthesize ADP-glucose from Glucose-1 phosphate and ATP, in a plant cell in vivo. Knock-out mutant AGPase alleles include, for instance, insertion and/or deletion mutations that disrupt the coding region, delete a portion or the entire coding region, and frameshift or stop-codon mutations that lead to a substantial or entire deletion of the protein. For example, a knock-out APS allele may comprise a mutation that disrupts or deletes the regulatory sequence containing the allosteric activator, glycerate 3-phosphate, binding site (e.g., see FIG. 1C). Any stop codon, frame shift, or splice site mutation that leads to a C-terminal truncation of the APS protein that includes the allosteric activator, glycerate 3-phosphate, binding site will result in a full knock-out APS allele.

An exemplary mutation in Solanum lycopersicum APS1 causing a reduction or elimination of the expression, stability, and/or activity of AGPase protein, which is effective in reducing or eliminating starch synthesis and imparting blossom end rot resistance to fruit, comprises a deletion of the T residue at nucleotide position 1,321 of SEQ ID NO:2 resulting in a frameshift mutation (1321delT; FIG. 1C). The nucleotide sequence of the Solanum lycopersicum APS1 mutation 1321delT is provided as SEQ ID NO:130, as follows: GCAGATACTACGAGACTGAT (SEQ ID NO:130).

In some embodiments, the APS gene is an APS1 gene comprising a deletion mutation corresponding to the deletion mutation of SEQ ID NO:130, resulting in a frameshift mutation.

Other exemplary knock-out mutations include substitution mutations, insertions, and/or deletions in the nucleotide sequence encoding the allosteric activator, glycerate 3-phosphate, binding site itself, i.e., nucleotides 1512-1563 of SEQ ID NO:2 (corresponding to amino acid positions 504-521 of SEQ ID NO:3), and in similar allosteric binding site regions of other APS genes described herein (see Table 13). Other regulatory regions of APS1 include the ATP binding site at nucleotides 558-582 of SEQ ID NO:2 (corresponding to amino acid positions 186-194 of SEQ ID NO:3), the catalytic site at nucleotides 638-666 of SEQ ID NO:2 (corresponding to amino acid positions 213-222 of SEQ ID NO:3), and the glucose-1 phosphate binding site starting at nucleotides 789-1062 of SEQ ID NO:2 (corresponding to amino acid positions 263-354 of SEQ ID NO:3), and similar sequences of regulatory regions in APS genes described herein (see, e.g., Table 13). Exemplary knock-out mutations of APL1 include substitution mutations, insertions, or deletions in the nucleotide sequence encoding the allosteric activator, glycerate 3-phosphate, binding site itself, i.e., nucleotides 1494-1545 of SEQ ID NO: 11 (corresponding to amino acid positions 498-515 of SEQ ID NO:12), and in similar allosteric binding site regions of other APL genes described herein (see, e.g., Table 13). Other regulatory regions of APL1 include the ATP binding site at nucleotides 537-561 of SEQ ID NO: 11 (corresponding to amino acid positions 179-187 of SEQ ID NO:12), the catalytic site at nucleotides 627-653 of SEQ ID NO:11 (corresponding to amino acid positions 209-218 of SEQ ID NO: 12), and the glucose-1 phosphate binding site starting at nucleotides 777-801 of SEQ ID NO: 11 (corresponding to amino acid positions 259-267 of SEQ ID NO:12), and in similar sequences of regulatory regions in APL genes described herein (see, e.g., Table 13).

In some embodiments, a mutation may be introduced by mutagenesis such as by treatment with a mutagenic agent. Any suitable mutagenic agent can be used for embodiments of the present disclosure. For example, mutagens creating point mutations, deletions, insertions, rearrangements, transversions, transitions, or any combination thereof may be used. Suitable radiation mutagens include, without limitation, ultraviolet light, x-rays, gamma rays, and fast neutrons. Suitable chemical mutagens include, but are not limited to, ethyl methanesulfonate (EMS), methylmethane sulfonate (MMS), N-ethyl-N-nitrosourea (ENU), triethylmelamine (TEM), N-methyl-N-nitrosourea (MNU), procarbazine, chlorambucil, cyclophosphamide, diethyl sulfate, acrylamide monomer, melphalan, nitrogen mustard, vincristine, dimethylnitrosamine, N-methyl-N′-nitro-nitrosoguanidine 25 (MNNG), nitrosoguanidine, 2-aminopurine, 7, 12 dimethyl-benz (a) anthracene (DMBA), ethylene oxide, hexamethylphosphoramide, bisulfan, diepoxyalkanes (diepoxyoctane (DEO), diepoxybutane (DEB), 2-methoxy-6-chloro-9 [3-(ethyl-2-chloro-ethyl)aminopropylamino] acridine dihydrochloride (ICR-170), sodium azide, formaldehyde, or combinations thereof.

In some embodiments, a mutation may be introduced by genome editing. Genome editing is a type of genetic engineering in which DNA is inserted, replaced, or removed, or any combination thereof, from a genome using artificially engineered nucleases or “molecular scissors.” The nucleases typically create double-stranded breaks (“DSBs”) at desired locations in the genome and harness the cell's endogenous mechanisms to repair the induced break by processes of homology dependent repair (“HDR”) or nonhomologous end-joining (“NHEJ”). Four main families of engineered nucleases include: Zinc finger nucleases (“ZFNs”), Transcription Activator-Like Effector Nucleases (“TALENs”), the Clustered Regularly Interspaced Short Palindromic Repeats (“CRISPR”) system, and engineered meganuclease with re-engineered homing endonucleases. Any method of genome editing may be used in the embodiments of the present application.

Use of systems for gene editing has been widely described. For example, the use of CRISPR guide RNA in conjunction with CRISPR/Cas technology to target RNA is described in Wiedenheft et al., “RNA-Guided Genetic Silencing Systems in Bacteria and Archaea,” Nature 482:331-338 (2012); Zhang et al., “Multiplex Genome Engineering Using CRISPR/Cas Systems,” Science 339:819-23 (2013); and Gaj et al., “ZFN, TALEN, and CRISPR/Cas-based Methods for Genome Engineering,” Cell 31:397-405 (2013), which are hereby incorporated by reference in their entirety.

CRISPR/Cas type RNA-guided endonucleases provide an efficient system for inducing genetic modifications in genomes of many organisms and can be used in the methods described herein to introduce one or more genetic modifications in a plant genome. Non-limiting examples of genome editing nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, Mad7, or homologs, modified versions, and endonuclease inactive versions thereof. An example of a fusion protein to Cas9 is a cytidine deaminase-Cas9 fusion protein used in cytidine base editing to mutate nucleotides in target genes without generating double-strand breaks as described in Komor et al., “Programmable Editing of a Target Base in Genomic DNA without Double-Stranded DNA Cleavage,” Nature 533:420-424 (2016), which is hereby incorporated by reference in its entirety.

There are two distinct components to a CRISPR/Cas system, a guide RNA and an endonuclease, such as Cas9. The guide RNA is a combination of the endogenous bacterial CRISPR RNA (“crRNA”) and trans-activating crRNA (“tracrRNA”) into a single chimeric guide RNA (“gRNA”) transcript. The gRNA combines the targeting specificity of the crRNA with the scaffolding properties of the tracrRNA into a single transcript. When the gRNA and Cas9 are expressed in the cell, the genomic target sequence can be modified or permanently disrupted. The gRNA/Cas9 complex is recruited to the target sequence by the base-pairing between the gRNA sequence which has a region of the complementarity to the target sequence in the genomic DNA. For successful binding of Cas9, the genomic target sequence must also contain the correct Protospacer Adjacent Motif (“PAM”) sequence immediately following the target sequence. The binding of the gRNA/Cas9 complex localizes the Cas9 to the genomic target sequence so that the wild type Cas9 can cut both strands of DNA causing a DSB. Cas9 generates DSBs through the combined activity of two nuclease domains, RuvC and HNH. Cas9 will cut 3-4 nucleotides upstream of the PAM sequence. A DSB can be repaired through one of two general repair pathways: (1) NHEJ DNA repair pathway or (2) the HDR pathway. The NHEJ repair pathway often results in insertions/deletions (InDels) at the DSB site that can lead to frameshifts and/or premature stop codons, effectively disrupting the open reading frame (ORF) of the targeted gene. The HDR pathway requires the presence of a repair template, which is used to fix the DSB. HDR faithfully copies the sequence of the repair template to the cut target sequence. Specific nucleotide changes can be introduced into a targeted gene by the use of HDR with a repair template. CRISPR specificity can be controlled by level of homology and binding strength of the specific gRNA for a given gene target, or by modification of the Cas endonuclease itself. For example, a D10A mutant of the RuvC domain, retains only the HNH domain and generates a DNA nick rather than a DSB.

When the guide RNA and the gene editing endonuclease are expressed in the cell, the genomic target sequence can be modified and/or permanently disrupted. The guide RNA/gene editing endonuclease complex is recruited to the target sequence by the base-pairing between the guide RNA sequence and the complementary sequence of the target sequence in the genomic DNA.

In some embodiments, CRISPR gene editing is used to generate an AGPase gene mutation by causing nucleotide insertions or deletions (indels) at the DSB site. In some embodiments, a point mutation, insertions, deletions, or any combination thereof, can be generated in an AGPase gene. In yet other embodiments, one or more AGPase gene targets (such as one or more APS genes, one or more APL genes, or combinations of one or more APS and APL genes) can be modified using CRISPR gene editing in a single experiment using single or multiple guide RNAs having specificity for the different gene targets. In some embodiments, two AGPase gene targets are mutated. In other embodiments, more than two AGPase genes are mutated. Thus, a wide range of genetic modifications using the described methods and CRISPR gene editing can be attained.

In some embodiments, one or more mutations reduce AGPase gene expression by 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% (i.e., no expression product is produced by the cell), or any number or range therein, as compared to the amount of AGPase gene expression in a control. In some embodiments, one or more mutations reduce APS gene expression by 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% (i.e., no expression product is produced by the cell), or any number or range therein, as compared to the amount of APS gene expression in a control. In further embodiments, one or more mutations reduce APL gene expression by 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% (i.e., no expression product is produced by the cell), or any number or range therein, as compared to the amount of APL gene expression in a control.

In some embodiments, one or more mutations reduce the amount of functional AGPase protein by at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% (i.e., no functional AGPase protein having the ability to synthesize ADP-glucose from Glucose-1 phosphate and ATP is produced by the cell), or any number or range therein, as compared to the amount of the functional AGPase protein produced by the cell not comprising the mutant AGPase allele. Also encompassed is (i) the production of a “non-functional” AGPase protein (e.g., a truncated AGPase protein) having no detectable biological activity in vivo; (ii) the reduction in the absolute amount of the functional AGPase protein (e.g., reduced amount of AGPase protein being made due to the mutation in the AGPase gene); or (iii) the production of an AGPase protein with significantly reduced detectable biological activity (such as an AGPase protein in which one or more regulatory amino acid residues that are crucial for the biological activity of the encoded AGPase protein, are substituted or deleted), each as compared to the activity of a functional wild type (or unmodified) AGPase protein. In some embodiments, one or more mutations reduce the amount of functional APS protein by at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% (i.e., no functional protein is produced by the cell) or any number or range therein, as compared to the amount of the functional APS protein produced by the cell not comprising the mutant APS allele. In some embodiments, one or more mutations reduce the amount of functional APL protein by at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% (i.e., no functional protein is produced by the cell) or any number or range therein as compared to the amount of the functional APL protein produced by the cell not comprising the mutant APL allele.

### Inhibitory Polynucleotides

In some embodiments, the methods of the present disclosure further comprise introducing into the plant or plant cell an inhibitory polynucleotide targeting the AGPase gene encoding the AGPase protein. A plant or plant cell of this and other aspects of the present disclosure may comprise an inhibitory polynucleotide targeting one or more AGPase genes to reduce their expression. Methods for reducing gene expression involving the expression of “inhibitory polynucleotide” sequences in plants are known in the art, and include, but are not limited to, cosuppression, antisense suppression, double-stranded RNA (dsRNA) interference, hairpin RNA (hpRNA) interference, intron-containing hairpin RNA (ihpRNA) interference, transcriptional gene silencing, and micro RNA (miRNA) interference, as non-limiting examples.

For example, an inhibitory polynucleotide may be an antisense polynucleotide or an RNA inhibitor (RNAi) polynucleotide capable of down-regulating gene expression of one or more APS and/or APL subunits of AGPase.

RNA interference (RNAi) occurs when an organism recognizes double-stranded RNA molecules and hydrolyzes them. The resulting hydrolysis products are small RNA fragments of 19-24 nucleotides in length, called small interfering RNAs (siRNAs) or microRNAs (miRNAs). The siRNAs then hybridize to mRNAs (or other RNAs) and cause hydrolysis of the RNA. Most plant miRNAs show extensive base pairing to, and guide cleavage of their target mRNAs.

The RNAi pathway can be exploited in plants by using recombinant technology, which entails transforming a plant with a vector comprising DNA that when expressed produces a dsRNA homologous or nearly homologous to a gene target. The gene target can be homologous to an endogenous plant RNA. RNA interference in plants can also be referred to as post-transcriptional gene silencing or RNA silencing and can be triggered by expression of an antisense strand of a target sequence of interest. In general, a plant is transformed with DNA that is incorporated into the plant genome, and when expressed produces a dsRNA that is complementary to a gene of interest, which can be an endogenous plant gene (e.g., AGPase), leading to the reduction or elimination of expression of that gene.

In some embodiments, the inhibitory polynucleotide is an RNAi polynucleotide comprising a sense polynucleotide strand comprising at least 20 contiguous nucleotides from an AGPase gene sequence, and an antisense polynucleotide strand that hybridizes to the sense polynucleotide strand, where the sense and the antisense polynucleotide strand form a duplex. In some embodiments, the RNAi polynucleotide comprises a sense polynucleotide strand comprising at least 20, 30, 40, 50, 60, 70, 80, 90, 100, 200, 300, 400 or more than 400 contiguous nucleotides from an AGPase sequence, and an antisense polynucleotide strand that hybridizes to the sense polynucleotide strand, wherein the sense and the antisense polynucleotide strand form a duplex. In some embodiments, the RNAi polynucleotide comprises a sense polynucleotide strand 20-400 contiguous nucleotides from an AGPase sequence, and an antisense polynucleotide strand that hybridizes to the sense polynucleotide strand, wherein the sense and the antisense polynucleotide strand form a duplex.

RNAi constructs may also include a polynucleotide sequence that separates the sense and antisense strands to facilitate duplex formation. In some embodiments, the RNAi polynucleotide further comprises a polynucleotide sequence heterologous to the sense and antisense polynucleotides. In some embodiments, the RNAi polynucleotide comprises an intron.

In some embodiments, the inhibitory polynucleotide comprises an antisense polynucleotide. In some embodiments, the inhibitory polynucleotide comprises an antisense polynucleotide of an AGPase coding sequence of at least 20 base pairs (bp), 25 bp, 30 bp, 35 bp, 40 bp, 45 bp, 50 bp, 55 bp, 60 bp, 65 bp, 70 bp, 75 bp, 80 bp, 85 bp, 90 bp, 95 bp, 100 bp, 125 bp, 150 bp, 175 bp, 200 bp, 300 bp, 400 bp, 500 bp, 600 bp, 700 bp, 800 bp, 900 bp, or greater than 1000 bp, or any number of base pairs or range of base pairs between 20 bp and 1000 bp.

In some embodiments, the inhibitory polynucleotide reduces AGPase gene expression by 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% (i.e., no expression product is produced by the cell), or any amount or range therein, as compared to the amount of AGPase gene expression in a control. In some embodiments, the inhibitory polynucleotide reduces APS gene expression by 30%, 40%, 50%, 60%, 70%, 80%, 90%, 95% or 100% (i.e., no expression product is produced by the cell), or any number or range therein, as compared to the amount of APS gene expression in a control. In further embodiments, the inhibitory polynucleotide reduces APL gene expression by 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% (i.e., no expression is produced by the cell), or any number or range therein, as compared to the amount of APL gene expression in a control.

In some embodiments, the inhibitory polynucleotide reduces the amount of functional AGPase protein by at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% (i.e., no functional protein is produced by the cell) as compared to the amount of the functional AGPase protein produced by the cell not comprising the inhibitory polynucleotide. In some embodiments, the inhibitory polynucleotide reduces the amount of functional APS protein by at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% (i.e., no functional protein is produced by the cell), or any number or range therein, as compared to the amount of the functional APS protein produced by the cell not comprising the mutant APS allele. In some embodiments, the inhibitory polynucleotide reduces the amount of functional APL protein by at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% (i.e., no functional protein is produced by the cell) or any number or range therein, as compared to the amount of the functional APL protein produced by the cell not comprising the mutant APL allele.

### Plant Cells, Plants, & Fruits

In some embodiments, a plant comprises the plant cell of any of the embodiments of the present disclosure. Also provided is the fruit of a plant of any of the embodiments disclosed herein.

In some embodiments, the fruit, plant, or plant cell is from the Solanaceae family. In some embodiments, the fruit, plant, or plant cell is tomato (Solanum lycopersicum).

As used herein, a “fruit” is the mature ovary of a flowering plant that is typically cdible. In some embodiments, the fruit is a tomato fruit.

In some embodiments, the type of a tomato fruit is a cherry, bell, blocky, currant, deep round oval or roma, elongated, flattened globe, grape, hybrid, heirloom, long blocky, long pointed, oxheart, pear, beefsteak, round, small pear, small pointed, stuffer, tomatillo, processing, or plum shape.

In some embodiments, a tomato fruit has a skin colour dark, bi-color, deep pink, golden, green, orange, pink, red, white, or yellow color.

In some embodiments, the tomato plant is indeterminate, determinate, or dwarf. In some embodiments the tomato plant is an open-pollinated or hybrid plant.

In some embodiments, the tomato cultivar is, without limitation, Alicante, Amish Paste, Aunt Ruby's German Green, Azoychka, Beefsteak, Berkeley Tie-Dye Green, Better Boy, Big Beef, Big Mama, Big Rainbow, Blaby Special, Black Beauty, Black Cherry, Black Icicle, Black Krim, Brandywine, Breonice, Campari, Canario, Carbon, Celebrity, Cherokee Purple, Cherry Bambelo, Cherry Nebula, Chocolate Pear, Dad's Sunset, Dester, Dr. Wyche's Yellow, Early Girl, Ed's Millennium, Emerald Evergreen, Enchantment, Ferreira, Ferris Wheel, Flamenco, Fourth of July, Garden Peach, Gardener's Delight, German Johnson, German Lunchbox, German Pink, Giulietta F1, Granadero, Great White, Green Doctors, Green Giant, Green Zebra, Hanover tomato, Henderson's Pink Ponderosa, Heinz, Hillbilly, Hungarian Heart, Japanese Black Trifele, Jersey Boy, Jubilee, Juliet, Kellogg's Breakfast, Kentucky Beefsteak, Kumato, Lillian's Yellow, Malakhitovaya Shkatulka, Matt's Wild Cherry, McDreamy, Micro Tom, Millionaire, Moneymaker, Monterosa, Montserrat, Mortgage Lifter, Mr. Stripey, Mushroom Basket, M82, Napa Rose Blush, Orange Hat, Orange Icicle, Pantano Romanesco, Paul Robeson, Pink Boar, Plum tomato, Raf tomato, Raspberry Lyanna, Rebekah Allen, Rebellion, Red Currant, Roma, Rosa de Barbastro, Rosella, Rutgers, San Marzano, Santorini, Sasha Altai, Scorpio (Skorpion), Stupice, Super Sweet 100, Thorburn's Terra-cotta, Tigerella, Tiny Tim, Tomaccio, Tomkin, Traveller/Arkansas Traveler, True Black Brandywine, Violet Jasper (Tzi Bi U), Viva Italia, Wagner Blue Green, White Queen, Yellow Brandywine, Yellow Pear, or hybrid thereof. In some embodiments, the tomato cultivar is not Condine Red or Ailsa Craig.

In some embodiments, the fruit, plant, or plant cell is from the Solanaceae family. In some embodiments, the fruit, plant, or plant cell is from the Cucurbitaceac family. In some embodiments, the fruit, plant, or plant cell is from the Solanaceae or Cucurbitaceae family. In some embodiments, the fruit, plant, or plant cell is from the genus Solanum, Capsicum, Cucurbita, Cucumis, or Citrullus. In some embodiments, the fruit, plant, or plant cell is from the genus Solanum. In some embodiments, the fruit, plant, or plant cell is from the genus Capsicum, Cucurbita, Cucumis, or Citrullus. In some embodiments, the fruit or plant is, or the plant cell is from, a tomato, eggplant, pepper, squash, zucchini, cucumber, or melon. In some embodiments, the fruit or plant is (or plant cell is from) an eggplant, pepper, zucchini, cucumber, watermelon, pumpkin, squash, or melon. Exemplary melons include, without limitation, calabash, bitter melon, banana melon, crane melon, cantaloupe, Charentais melon, honeydew, winter melon, canary melon, and muskmelon. Exemplary squashes include, without limitation, butternet, crookneck, acorn, banana, butternet, carnival, delicata, and spaghetti.

### Blossom End Rot, Fruit Size, & Antioxidants

Blossom end rot can occur in response to environmental stress such as drought, or due to other factors, such as nutritional stress. In some embodiments, the fruit of the modified plants of the present disclosure has reduced levels of blossom end rot as compared to fruit from a plant without the modification. A plant without the modification as described herein may be, e.g., a wildtype plant. In some embodiments, blossom end rot is reduced in the fruit by 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100% (i.e, no blossom end rot), or any number or range therein, as compared to the amount of blossom end rot in fruit from a plant without the modification. In some embodiments, the fruit has reduced or no blossom end rot under normal growth or water stress conditions as compared to fruit from a plant without the modification. As used herein, “water stress conditions” are conditions in which the plant is grown in a pot with a volumetric water content (“VWC”) of less than 40-50% VWC or in a field or garden with 10-95% less water than is typically provided to a crop. In some embodiments, the water stress conditions comprise irregular watering. In some embodiments, the water stress conditions comprise pot VWC of 35%, 30%, 25%, 20%, 15%, 10%, 5%, or any number or range of VWC therein. In some embodiments, water stress conditions occur during fruit development. In some embodiments, water stress conditions occur in the period from 0 dpa (days post anthesis) to 30 dpa.

In some embodiments, the plant of the present disclosure is grown indoors. In some embodiments, the plant is grown hydroponically or acroponically. In some embodiments, the plant is grown outdoors (e.g., in a field or a garden).

Another aspect of the present disclosure relates to a method of increasing size of fruit from a plant. The method comprises modifying a plant or a plant cell to reduce or eliminate the expression, stability, and/or activity of an ADP-glucose pyrophosphorylase (AGPase) protein, where the reduction or elimination of the expression, stability, and/or activity of AGPase protein is effective in reducing or eliminating starch synthesis and increasing fruit size in fruit from the plant or a plant produced from the plant cell, as compared to fruit from a plant without the modification.

This aspect of the present disclosure can be carried out with any of the embodiments disclosed herein.

In some embodiments, the fruit of the modified plants of the present disclosure has increased fruit size as compared to fruit from a plant without the modification. In some embodiments, increased fruit size comprises an increase in fruit weight. In some embodiments, the fruit of the modified plants has an increase in fruit weight of at least 5%, 6%, 7%, 8%, 9%, 10%, 11%, 12%, 13%, 14%, 15%, or any number or range therein, as compared to fruit from a plant without the modification. In some embodiments, the increased fruit size comprises an increase in fruit weight by at least 10% compared to fruit from a plant without the modification. In some embodiments, increased fruit size comprises an increase in fruit volume. In some embodiments, the fruit of the modified plants has an increase in fruit volume of at least 1%, 2%, 3%, 4%, 5%, 6%, 7%, 8%, 9%, 10%, 11%, 12%, 13%, 14%, 15%, or any number or range therein, as compared to fruit from a plant without the modification. In some embodiments, the increased fruit size comprises an increase in fruit volume by at least 5% compared to fruit from a plant without the modification.

A further aspect of the present disclosure relates to a method of increasing antioxidants in fruit from a plant. The method comprises modifying a plant or a plant cell to reduce or eliminate the expression, stability, and/or activity of an ADP-glucose pyrophosphorylase (AGPase) protein, where the reduction or elimination of the expression, stability, and/or activity of AGPase protein is effective in reducing or eliminating starch synthesis and increasing antioxidant levels in a fruit from the plant or a plant produced from the plant cell, as compared to a fruit from a plant without the modification.

This aspect of the present disclosure can be carried out with any of the embodiments disclosed herein.

Antioxidants are substances that can remove or inhibit potentially damaging oxidation. In some embodiments, the fruit of the modified plants of the present disclosure has increased antioxidants compared to fruit from a plant without the modification. In some embodiments, the increased antioxidants comprise raffinose, phenolics, and/or pyridoxal. In some embodiments, the antioxidants are increased by 1.1-fold to 3-fold, or any number or range therein, as compared to fruit from a plant without the modification.

Another aspect of the present disclosure relates to a plant cell comprising a nucleic acid molecule comprising a modified ADP-glucose pyrophosphorylase (AGPase) gene encoding an AGPase protein or fragment thereof, wherein the expression, stability, and/or activity of the AGPase protein is reduced or eliminated in the plant cell as compared to a plant cell without the modification.

Yet another aspect of the present disclosure relates to a plant cell comprising an inhibitory polynucleotide targeting an AGPase gene encoding an AGPase protein, wherein the expression, stability, and/or activity of the AGPase protein is reduced or eliminated in the plant cell as compared to a plant cell without the inhibitory polynucleotide.

These aspects of the present disclosure can be carried out with any of the embodiments disclosed herein.

### Nucleic Acid Constructs

A further aspect of the present disclosure relates to a nucleic acid construct comprising a nucleotide sequence targeting an AGPase gene comprising: (i) a guide RNA, or (ii) an inhibitory polynucleotide, a 5′ heterologous DNA promoter sequence; and a 3′ terminator sequence.

In some embodiments, the guide RNA targets one or more AGPase genes, APS genes, and/or APL genes. In some embodiments, the inhibitory polynucleotide targets one or more AGPase genes, APS genes, and/or APL genes.

A “nucleic acid construct” or “vector” is a nucleic acid, plasmid, or virus used to transfer coding information to a host cell. Typically, the vector contains sequences directing transcription and translation of the relevant gene, a selectable marker, and sequences allowing autonomous replication or chromosomal integration. Suitable “expression vectors” comprise a region 5′ of the gene which harbors transcriptional initiation controls and a region 3′ of the DNA fragment which controls transcriptional termination.

The DNA sequence in the expression vector is operably linked to appropriate expression control sequences, including a promoter, to direct RNA synthesis and protein expression. “Operably linked” means an association between polynucleotide (e.g., nucleic acid) sequences on a single nucleic acid molecule such that the function of one is affected by the other. For example, a promoter is operably linked to a coding sequence when the coding sequence is under the transcriptional control of the promoter. Coding sequences can be operably linked to regulatory sequences in sense or antisense orientation. The expression vector can contain one or more selectable marker genes to provide a phenotypic trait for selection of a transformed host cell. Useful selectable markers include dihydrofolate reductase or neomycin resistance for eukaryotic cell culture, or tetracycline or ampicillin resistance in E. coli. The vector may be introduced into the host cell(s) using any of a variety of techniques, including transformation, transfection, transduction, viral infection, gene guns, Ti-mediated gene transfer, calcium phosphate transfection, DEAE-Dextran-mediated transfection, lipofection, or electroporation. Examples of vectors include, but are not limited to, viral particles, baculovirus, phage, plasmids, phagemids, cosmids, fosmids, bacterial artificial chromosomes, viral DNA, such as vaccinia and adenovirus, Ti vectors, P1-based artificial chromosomes, yeast plasmids, Bacillus vectors, and Aspergillus vectors. Examples of bacterial vectors include, but are not limited to, pQE vectors, pBluescript plasmids, pNH vectors, and lambda-ZAP vectors. Examples of eukaryotic vectors include pXT1, pSG5, pSVK3, pBPV, pMSG, and pSVLSV40 vectors.

U.S. Pat. No. 4,237,224 to Cohen and Boyer, which is hereby incorporated by reference in its entirety, describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

Depending upon the targeted expression profile in a cell or tissue type, or developmental timeframe, any one of a number of suitable promoters may be used. A “promoter” refers to a nucleic acid fragment capable of controlling transcription of another nucleic acid fragment. A promoter is a non-coding genomic DNA sequence, usually upstream (5′) to and operably linked to the relevant coding sequence, and its primary function is to act as a binding site for RNA polymerase to initiate transcription by the RNA polymerase. A promoter may also include distal enhancer or repressor elements, which can be located as much as several thousand base pairs from the start site of transcription. The terms “capable of controlling expression” or “initiating transcription”, refer to the primary function of a promoter. Additionally, there is “expression” of RNA, including functional RNA, or the expression of polypeptide for operably linked encoding nucleotide sequences, as the transcribed RNA ultimately may be translated into the corresponding polypeptide. Promoters vary in their “strength” (i.e., their ability to promote transcription). The nucleotide sequence of the promoter determines the nature of the RNA polymerase binding and other related protein factors that attach to the RNA polymerase and/or promoter, and the rate of RNA synthesis.

A “constitutive” promoter is a promoter that is active under most environmental and developmental conditions. An “inducible” promoter is a promoter that is active under environmental or developmental regulation. Suitable constitutive promoters that are functional in a plant cell include, but are not limited to, the cauliflower mosaic virus 35S (CaMV35S) promoter, a tandem 35S promoter, a cauliflower mosaic virus 19S promoter, a figwart mosaic virus 35S (FMV35S) promoter, a nopaline synthase gene promoter, an octopine synthase gene promoter, a potato or tomato protease inhibitor I or II gene promoter, and a ubiquitin promoter. Suitable inducible promoters that are functional in a plant cell may include, but are not limited to, a phenylalanine ammonia-lyase gene promoter, a chalcone synthase gene promoter, a pathogenesis-related protein gene promoter, a copper-inducible regulatory element, tetracycline, and chlor-tetracycline-inducible regulatory elements.

A “tissue-specific” promoter is a promoter that primarily are functional in a specific tissue type. For example, tissue specific promoters can be employed which regulate expression in only one or some tissues or organs, such as leaves, roots, fruit, seeds, anthers, ovaries, pollen, meristem, stems or flowers, or parts thereof. Methods for identifying and characterizing promoter regions in plant genomic DNA include, for example, those described in the following references: Jordano et al., Plant Cell, 1:855-866 (1989); Bustos et al., Plant Cell, 1:839-854 (1989); Green et al., EMBO J., 7:4035-4044 (1988); Meier et al., Plant Cell, 3:309-316 (1991); and Zhang et al., Plant Physiology, 110:1069-1079 (1996). Non-limiting examples of tissue-specific promoters include, without limitation, B29 promoter, CD14 promoter, CD43 promoter, CD45 promoter, CD68 promoter, desmin promoter, elastase-1 promotor, endoglin promoter, fibronectin promoter, Flt-1 promoter, GFAP promoter, GPIIb promoter, ICAM-2 promoter, INF-(3 promoter, Mb promoter, NphsI promoter, OG-2 promoter, SP-B promoter, SYN1 promoter, and WASP promoter.

The tissue-specific ES promoter from tomato is particularly useful for directing gene expression so that a desired gene product is located in fruits (see, e.g., Lincoln et al. “Diverse Mechanisms for the Regulation of Ehylene-Inducible Gene Expression,” Proc Natl Acad Sci USA 84:2793-2797 (1988), which is hereby incorporated by reference in its entirety). An example of a seed-specific promoter is the promoter from the gene encoding oleosin from Arabidopsis (Genbank Accession No. Z17657, which is hereby incorporated by reference in its entirety). Other exemplary promoters are described in Fernandez et al., “Flexible Tools for Gene Expression and Silencing in Tomato,” Plant Physiology 151:1729-1740 (2009) (which is hereby incorporated by reference in its entirety). In some embodiments, the 5′ heterologous DNA promoter sequence is a tissue-specific promoter. In certain embodiments, the tissue-specific promoter is a promoter identified on the ePlant database (available on the world wide web at bar.atoronto.ca/eplant_tomato, incorporated herein by reference) or on the Melonet database (available on the world wide web at melonnet-db.dna.affre.go.jp/ap/mvw, incorporated herein by reference).

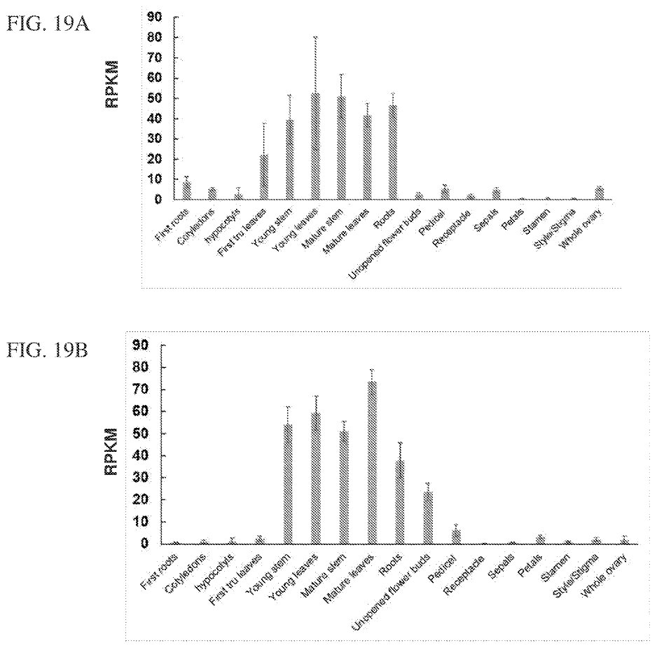

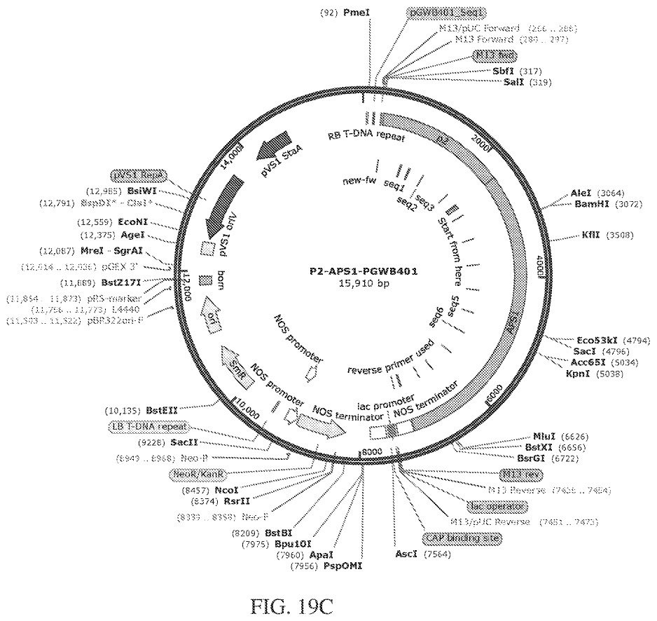

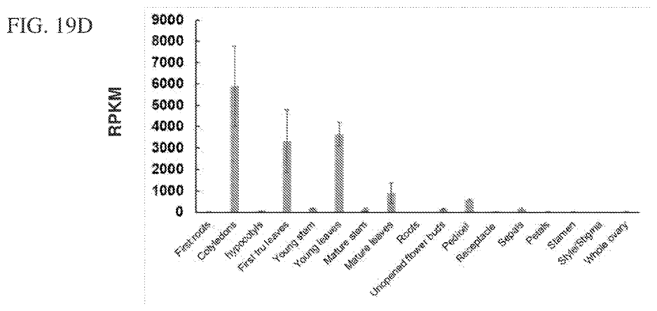

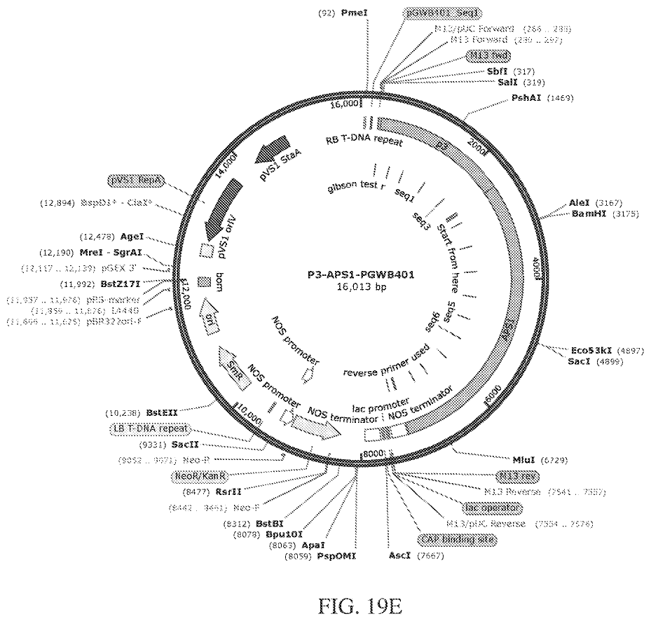

In certain embodiments, the tissue-specific promoter is a promoter of the tomato gene STM3 (Solyc01g092950) which is mostly expressed in leaf and root tissues, but not in fruits. In certain embodiments, the tissue-specific promoter is a promoter of the tomato gene SIMBP24 (Solyc01g105800) which is mostly expressed in leaf and root tissues, but not in fruits, The Solyc01g105800 tissue specific promoter has been assembled with the APS1 into the pGWB401 backbone (See FIG. 19A). In certain embodiments, the tissue-specific promoter is a promoter of the tomato gene βCA1 (Solyc02g086820) which is mostly expressed in leaf tissues, but not in fruits. The Solyc02g086820 tissue specific promoter has been assembled with the APS1 into the pGWB401 backbone (See FIG. 19B).

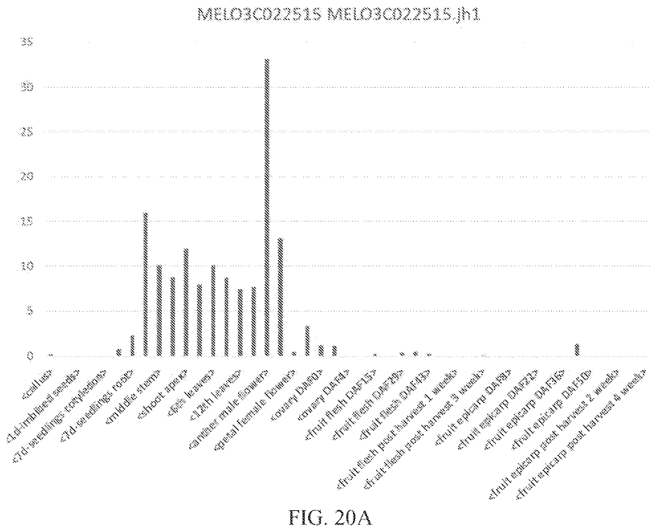

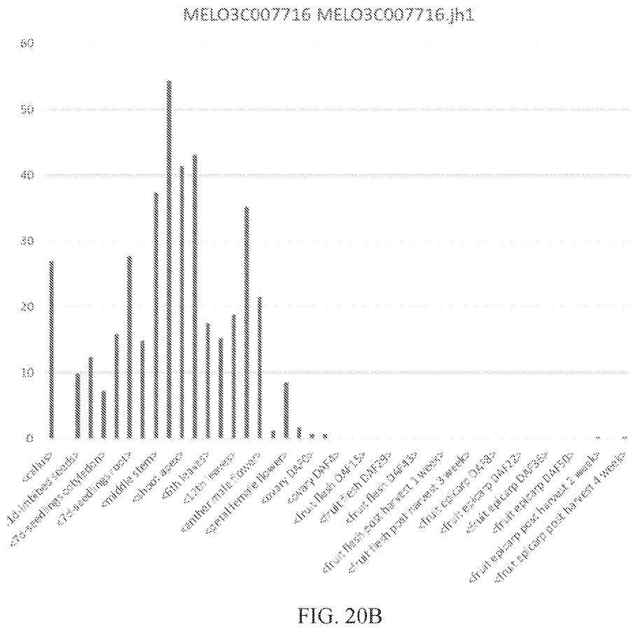

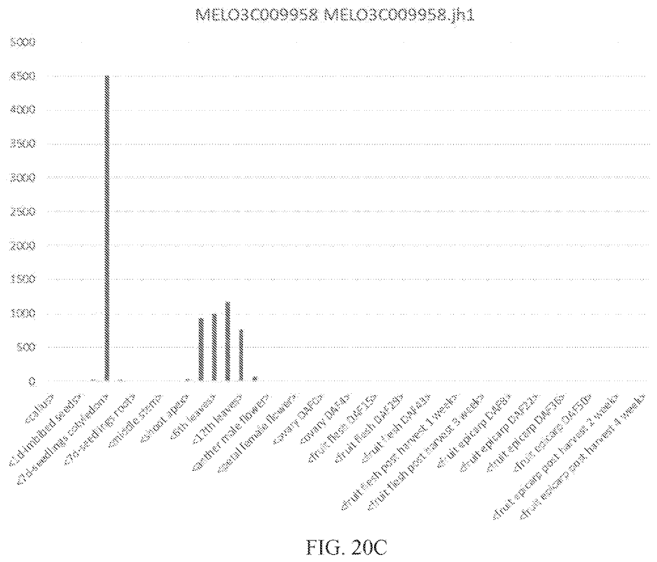

In certain embodiments, the tissue-specific promoter is a promoter of melon gene MELO3C022515 which is primarily expressed in plant flowers (FIG. 20A). In certain embodiments, the tissue-specific promoter is a promoter of melon gene MELO3C007716 which is primarily expressed in plant leaves stems and roots (FIG. 20B). In certain embodiments, the tissue-specific promoter is a promoter of melon gene MELO3C00958 which is primarily expressed in plant seeds and leaves (FIG. 20C), Other exemplary tissue-specific promoter can be found in Table 14.

TABLE 14
Tissue Specific Promoters

| Promoter | Tissue of Expression |
|---|---|
| Phosphoenolypyruvate | Fruit |
| Carboxylase 2 (PPC2) | |
| promoter | |
| Tomato Proline Rich | Fruit |
| Protein (TPRP) | |
| Promoter | |
| Inhibitor of Meristem | Fruit (Accesssion |
| Activity (IMA) | number AC122544) |
| Promoter | |
| Arabidopsis CRABS | Fruit |
| CLAW (CRC) | |
| promoter | |
| Tomato | Fruit |
| Polyhglacturonasc | |
| (PG) promoter | |
| P_{RSGA} | Seed |
| P_{2RSGA} | Seed |
| BiGSSP2 | Green Tissues |
| BiGSSP3 | Green Tissues |
| BiGSSP6 | Green Tissues |
| BiGSSP7 | Green Tissues |
| SynR2 | Root |
| SynR1 | Root |
| GSSP1 | Green Tissues |
| GSSP3 | Green Tissues |
| GSSP5 | Green Tissues |
| GSSP6 | Green Tissues |
| GSSP7 | Green Tissues |
| P35S-PCHS-Ω | Flower |
| P35S-LCHS-Ω | Flower |
| EFCFS-HS-1 | Vascular Tissues |
| EFCHFS-HS-1 | Vascular Tissues |
| EFCFSHS-3 | Vascular Tissues |

Promoters may also be plant-specific promoters, or promoters that function in plants. A wide variety of plant promoters are known to those of ordinary skill in the art, as are other regulatory elements that may be used alone or in combination with promoters.

The protein-encoding nucleic acid, a promoter molecule of choice, a suitable 3′ regulatory region and, if desired, polyadenylation signals and/or a reporter gene, are incorporated into a vector-expression system of choice to prepare a nucleic acid construct using standard cloning procedures known in the art, such as described by Sambrook et al., Molecular Cloning: A Laboratory Manual, Third Edition, Cold Spring Harbor: Cold Spring Harbor Laboratory Press, New York (2001), which is hereby incorporated by reference in its entirety.

Single or multiple nucleic acids may be ligated into an appropriate vector, under the control of one or more suitable promoters, to prepare a nucleic acid construct. In some embodiments, the expression vector comprising the nucleic acid construct. In some embodiments, the expression vector further comprises a genome editing nuclease.

Once the nucleic acid molecule has been inserted into an expression vector, it is ready to be incorporated into a host cell. Recombinant molecules can be introduced into cells via transformation, particularly transduction, conjugation, lipofection, protoplast fusion, mobilization, particle bombardment, or electroporation. “Transformation” or “transforming” refers to the introduction of a nucleic acid into a host organism. Host organisms containing a transformed nucleic acid construct or DNA fragment may be referred to as “transgenic” or “recombinant” organisms.

In some embodiments, a cell is transformed with the nucleic acid construct. In some embodiments, the cell is a bacterial cell or a plant cell. A transformed plant cell may be regenerated into a plant by any method known in the art. In some embodiments, a plant is transformed with the nucleic acid construct. Fruit from the plant or a plant grown from the plant cell of any of the embodiments of the present disclosure are also contemplated. In some embodiments, a plant seed produced from the plant of any of the embodiments of the present disclosure is contemplated.

### Plant Breeding

Another aspect of the present disclosure relates to a method of breeding for enhanced blossom end rot resistance. The method comprises providing a candidate plant, analyzing the candidate plant for the presence, in its genome, of a modified ADP-glucose pyrophosphorylase (AGPase) polynucleotide encoding an AGPase protein, wherein the expression, stability, and/or activity of said AGPase protein is reduced or eliminated as compared to a plant without the modification, identifying, based on said analyzing, a candidate plant suitable for breeding that includes in its genome, the modified ADP-glucose pyrophosphorylase (AGPase) polynucleotide, and breeding the identified plant with at least one other plant.

This aspect of the present disclosure can be carried out with any of the embodiments disclosed herein.

In some embodiments, identifying the presence of a modified AGPase polynucleotide sequence is achieved based on sequencing the sequences set forth herein, by identifying a sequence based on homology to the sequences set forth herein. In some embodiments, a molecular marker is developed to identify a modified AGPase polynucleotide sequence. Sequencing can be accomplished, for example, using next generation sequencing, Sanger sequencing, TaqMan assays, UniTaq assays, real-time PCR assays, digital PCR, microarray, hybridization or other detection methods.

In some embodiments, analyzing the candidate plant for the presence, in its genome, of a modified ADP-glucose pyrophosphorylase (AGPase) polynucleotide encoding an AGPase protein, wherein the expression, stability, and/or activity of said AGPase protein is reduced or eliminated as compared to a plant without the modification involves isolating nucleic acids from the plant or plant part, analyzing nucleic acids from the plant, or plant parts for the presence of the modified AGPase polynucleotide, and detecting the modified AGPase polynucleotide.

In some embodiments, identifying comprises sequencing the nucleic acids of one or more AGPases from the plant, or plant part. In some embodiments, identifying comprises detecting a mutation corresponding to 1321delT of SEQ ID NO:130. In some embodiments, identifying comprises detecting the nucleotide sequence of SEQ ID NO:130. In some embodiments, analyzing the candidate plant for a modified AGPase involves testing starch content. In some embodiments, testing starch content involves staining with iodine or Lugol's reagent. In some embodiments, said identifying comprises detecting a plant or fruit with no or low staining with iodine or Lugol's reagent, compared to a plant without a modified AGPase. In some embodiments, testing starch content involves quantifying starch enzymatically.

In some embodiments, the breeding involves crossing, making hybrids, backcrossing, self-crossing, double haploid breeding, and/or combinations thereof.

### Selective Expression of AGPase

Yet another aspect of the present disclosure relates to a method for imparting blossom end rot resistance to fruit from a plant. The method comprises modifying a plant or a plant cell to reduce or eliminate the expression, stability, and/or activity of ADP-glucose pyrophosphorylase (AGPase) protein, where the reduction or elimination of the expression, stability, and/or activity of AGPase protein is effective in reducing or eliminating starch synthesis and imparting blossom end rot resistance to fruit from the plant or a plant produced from the plant cell, as compared to fruit from a plant without the modification, and selectively expressing a functional AGPase protein in non-fruit tissues.

This aspect of the present disclosure can be carried out with any of the embodiments disclosed herein.

In some embodiments, the methods of the present disclosure comprise selectively expressing a functional AGPase in the non-fruit tissue of the plant. “Selectively expressed” or “selectively expressing” means that expression of the functional AGPase is targeted to a particular non-fruit tissue or tissue types. Selective expression may be performed by introducing a nucleic acid construct comprising a functional AGPase gene operably linked to a non-fruit specific promoter into a plant or plant cell. For example, a functional AGPase (unmutated or unmodified) could be expressed in non-fruit tissue (such as leaves) to restore starch synthesis in non-fruit tissues.

In some embodiments, a polynucleotide encoding a functional AGPase corresponding to the modified AGPase is operably linked to a non-fruit specific promoter (or a promoter that does not drive expression during fruit development) is introduced into the plant having a modified AGPase. For example, in an embodiment where a mutated APS1 gene is present, a polynucleotide encoding the functional APS1 gene operably linked to a non-fruit specific promoter (or a promoter that does not drive expression during fruit development) is introduced into the plant.

The following examples are provided to illustrate embodiments of the present disclosure but are by no means intended to limit its scope.

### Examples

### Example 1—Materials and Methods Used for Examples 2-6

Plant growth conditions and sample collection. Seed stocks for wild type (Solanum lycopersicum, LA2838A) and the adp mutant (S. lycopersicum, LA3763) were obtained from the Tomato Genetics Resource Center (TGRC) at the University of California, Davis. Tomato plants were grown in a greenhouse at Cornell University (Ithaca, NY, USA) with a light/dark photoperiod of 16/8 h. Soil moisture sensors (MAS-1, Decagon Devices, Pullman, USA) were used to monitor pot volumetric water content (VWC) and to regulate an irrigation system to maintain 40-50% VWC. Flowers were tagged and manually pollinated at anthesis, and fruits were harvested at 6 days post anthesis (dpa), 20 dpa, and at the mature green (MG, ~35 dpa) and ripe (breaker+8 days) stages, from 10 randomly selected individual plants per genotype.

To assess BER incidence, water stress was applied by lowering pot VWC to 30% (Nicolas et al “Spatiotemporal Dynamics of The Tomato Fruit Transcriptome Under Prolonged Water Stress,” Plant Physiology 190:2557-2578 (2022), which is hereby incorporated by reference in its entirety) 5 to 7 days after flowers were tagged and pollinated. Five plants per genotype were used with at least 10 fruit per plant, and BER incidence was assessed in fruit at 30 dpa. The experiment was repeated three times.

Map-based cloning. To identify the gene responsible for the adp mutation, an F2 mapping population was generated by crossing adp to Solanum pimpinellifolium (LA1589), and this population was phenotyped for agravitropism at the seedling stage on vertical petri dishes. Approximately 200 agravitropic seedlings were genotyped using PCR markers, across chromosome 7, designed based on indels and SNP positions according to the tomato Heinz 1706 reference genome (The Tomato Genome Consortium, “The Tomato Genome Sequence Provides Insights into Fleshy Fruit Evolution,” Nature 485:635-641 (2012), which is hereby incorporated by reference in its entirety).

RNA isolation, sequencing and analysis. Dissected pericarp and placenta were ground in liquid nitrogen and total RNA was isolated using an RNeasy Mini Kit (Qiagen). Strand-specific libraries were constructed according to Zhong et al., “High-Throughput Illumina Strand-Specific RNA Sequencing Library Preparation,” Cold Spring Harb Protoc 8:940-9 (2011), which is hereby incorporated by reference in its entirety, and sequenced on an Illumina NextSeq 500 from a single end for 75 cycles. After removing adapter and low-quality sequences using Trimmomatic (Bolger et al., Trimmomatic: A Flexible Trimmer for Illumina Sequence Data,” Bioinformatics 30:2114-2120 (2014), which is hereby incorporated by reference in its entirety), RNA-Seq reads were aligned to the ribosomal RNA database (Quast et al., “The SILVA ribosomal RNA gene database project: improved data processing and web-based tools,” Nucleic Acids Res 41: D590-D596 (2013), which is hereby incorporated by reference in its entirety) using Bowtie (Langmead et al., “Ultrafast and Memory-Efficient Alignment of Short DNA Sequences to the Human Genome,” Genome Biol 10: R25 (2009), which is hereby incorporated by reference in its entirety) and the mappable reads discarded. The resulting high-quality cleaned reads were aligned to the tomato Heinz 1706 reference genome (The Tomato Genome Consortium, “The Tomato Genome Sequence Provides Insights into Fleshy Fruit Evolution,” Nature 485:635-641 (2012), which is hereby incorporated by reference in its entirety) SL 3.0 with gene models iTAG3.2 using HISAT (Kim et al., “HISAT: A Fast Spliced Aligner with Low Memory Requirements,” Nat Methods 12:357-360 (2015), which is hereby incorporated by reference in its entirety) and raw counts for each tomato gene were derived and normalized to reads per kilobase of transcript per million mapped reads (RPKM). Genes were considered expressed if their averaged RPKM among replicates was ≥1. Pairwise Pearson correlation coefficient values between biological replicates were calculated with log 2-transformed RPKM values (i.e., log 2 [RPKM+1]) using the cor function in the R program (www.r-project.org). Pairwise Pearson correlation coefficient values between biological replicates ranged from 0.88 to 1.00. PCA was performed to compare the log 2-transformed RPKM values of the expressed gene profiles among genotypes, tissue-types and stages, using the prcomp function in R v3.5.1. The log 2-transformed (RPKM+1) values of all the genes were used for hierarchical clustering using dist (method=“Euclidean”) function and hclust (method=“average”) function in R. Raw read count data were imported to edgeR (Robinson et al., “edgeR: A Bioconductor Package for Differential Expression Analysis of Digital Gene Expression Data,” Bioinformatics 26:139-140 (2010), which is hereby incorporated by reference in its entirety) to identify differentially expressed genes (DEGs) between each pair of adp and control samples with a cutoff of absolute fold change ≥1.5 and false discovery rate (FDR)<0.05. Gene annotations, iTAG3.2 gene ontology (GO) terms, were obtained from the PANTHER classification system version 10 (Mi et al., 2016). GO enrichment analysis was performed for GO Biological Process Complete using the Bioconductor R library clusterProfiler (Yu et al., “ClusterProfiler: An R Package for Comparing Biological Themes Among Gene Clusters,” OMICS 16:284-287 (2012), which is hereby incorporated by reference in its entirety), applying a hypergeometric test with FDR correction (adjusted p value <0.05).

Metabolite analysis. Soluble sugars and starch measurements were performed according to Vargas-Ortiz et al., “Grain Amaranths are Defoliation Tolerant Crop Species Capable of Utilizing Stem and Root Carbohydrate Reserves to Sustain Vegetative and Reproductive Growth After Leaf Loss,” PLOS One 8: e67879 (2013), which is hereby incorporated by reference in its entirety. Briefly, 50 mg of frozen tissue were extracted in 50 mM Hepes KOH (pH 7.4), 5 mM MgCl2, 80% ethanol (v/v), three times at 80° C. Soluble extracts were then combined and assayed enzymatically for glucose, fructose and sucrose in a microplate reader (Synergy 2, BioTeck Instruments). The insoluble starch pellets were dissolved in 0.25 ml 10 mM KOH at 95° C. for 1h. Starch was hydrolyzed in 50 mM sodium acetate buffer (pH 5.5) at 37° C. overnight by the addition of 10 units of α-amylase and 10 units of amyloglucosidase and solubilized sugars were enzymatically assayed for glucose, as mentioned above.

Metabolite extraction, derivatization, standard addition, and sample injection for GC-MS were performed according to Osorio et al., “Profiling Primary Metabolites of Tomato Fruit with Gas Chromatography/Mass Spectrometry,” Methods Mol Biol 860:101-109 (2012), which is hereby incorporated by reference in its entirety. The mass spectra were cross-referenced with those in the Golm Metabolome Database (Kopka et al., “GMD@CSB.DB: The Golm Metabolome Database,” Bioinformatics 21:1635-1638 (2005), which is hereby incorporated by reference in its entirety).

For untargeted LC-MS/MS analysis, frozen samples were extracted with 80% MeOH and homogenized in a Tissue Lyser for 3 min at 25/s. Methanolic extracts were centrifuged twice for 10 min at 12000 rpm and supernatants were stored at −80° C. until analysis. LC-MS/MS analysis was performed using orthogonal UPLC separation strategies (reversed phase and HILIC), in both positive and negative ionization modes to maximize identification of metabolites. Chromatographic separation was performed on a Vanquish UHPLC system coupled to a Q Exactive™ Hybrid Quadrupole-Orbitrap High Resolution Mass Spectrometer (Thermo Fisher Scientific, San Jose, CA, USA). A SeQuant ZIC PHILIC column (5 μm, 2.1×150 mm) and a Thermo Accucore Vanquish C18+, 1.5 μm column (2.1 mm id×100 mm) were used for the HILIC and Reversed phase methods, respectively. Acquired MS1 and DDMS2 files, in both positive and negative ion mode were processed separately in Compound Discoverer 2.1 using untargeted workflow (5 ppm mass tolerance and 0.1 min RT tolerance). Successful compound annotation was assessed using an in-house spectral library and references retrieved from the database mzCloud (www.mzcloud.org) with best match scores >80%.

Glycerolipids were analyzed using LC-MS according to Lapidot-Cohen et al., (2020). Briefly, samples were extracted using methyltert-butyl ether (MTBE) and injected in an ultra-performance liquid chromatography (UPLC) system (Waters Acquity UPLC) with a lipophilic column (BEH C81.7 μm) coupled to a Q Exactive mass spectrometer in positive and negative ionization modes (UPLC-MS).

Metabolomic data analyses (statistical analysis, PCA, hierarchical clustering and heat-maps) were performed using MetaboAnalyst 4.0 (Chong et al., “Using MetaboAnalyst 4.0 for Comprehensive and Integrative Metabolomics Data Analysis,” Curr Protoc Bioinformatics 68: e86 (2019), which is hereby incorporated by reference in its entirety).

Fluorescence Microscopy. Equatorial sections of tomato fruit were incubated with 1 μg/ml of the fluorophore BODIPY (4,4-difluoro-3a,4adiaza-s-indacene) 505/515 (ThermoFisher Scientific, USA) for 15 min, in the dark, and then rinsed three times with water. Samples were observed with a Leica M205 stereomicroscope using 505 nm and 515 nm as excitation and emission wavelengths, respectively.

Data Availability. Raw transcriptome sequencing reads have been deposited in the National Center for Biotechnology Information BioProject database under the accession no. PRJNA912612.

### Example 2—Characterization of Adpressa as a Starch-Deficient Tomato Mutant

The adpressa (adp) mutant (Solanum lycopersicum, LA3763) was initially isolated in the tomato cultivar Condine Red as a radiation induced mutation and described as having directionless hypocotyl growth and a crawling growth habit (Stubbe, “Mutanten der Kulturtomate Lycopersicon Esculentum Miller III. Die Kulturpflanze 7:82-112 (1959), which is hereby incorporated by reference in its entirety). The mutation was later introgressed into the Ailsa Craig cultivar (Solanum lycopersicum, LA2838A) by five backcrosses, and the adp gene was putatively assigned to chromosome 7 (Smith and Ritchie, “A Collection of Near-Isogenic Lines of Tomato: Research Tool of the Future?,” Plant Mol Biol Rep 1:41-45 (1983), which is hereby incorporated by reference in its entirety). Morphological analysis under greenhouse growth conditions showed that adp plants were shorter and with decumbent stem growth (FIG. 1A). In addition, adp seedlings grown in agar plates displayed agravitropic root growth (FIG. 1B).

To identify the adp gene, a mapping strategy using a F2 population derived from a cross between adp and the wild tomato Solanum pimpinellifolium (LA1589) was employed. The adp mutation mapped to an 86 Kb region of chromosome 7 that contained 11 genes including Solyc07g056140 encoding the ADP-glucose pyrophosphorylase (AGPase) small subunit, APS1 (FIG. 1C). AGPase catalyzes the conversion of glucose 1-phosphate and ATP into ADP-glucose and inorganic pyrophosphate, a rate-limiting step in the starch biosynthesis pathway (Geigenberger P., “Regulation of Starch Biosynthesis in Response to a Fluctuating Environment,” Plant Physiol 155:1566-1577 (2011), which is hereby incorporated by reference in its entirety). Given the role of starch in gravitropism (Morita and Tasaka, “Gravity Sensing and Signaling,” Curr Opin Plant Biol 7:712-718 (2004), which is hereby incorporated by reference in its entirety) this candidate gene was further investigated. Sanger sequencing of this genomic region identified a single base deletion close to the 3′ end of the AGPase coding sequence that resulted in a frame shift and premature stop codon, producing a protein missing 44 amino acids at the C-terminus (FIG. 1C), the region containing the allosteric activator, glycerate 3-phosphate, binding site (Geigenberger P., “Regulation of Starch Biosynthesis in Response to a Fluctuating Environment,” Plant Physiol 155:1566-1577 (2011), which is hereby incorporated by reference in its entirety).

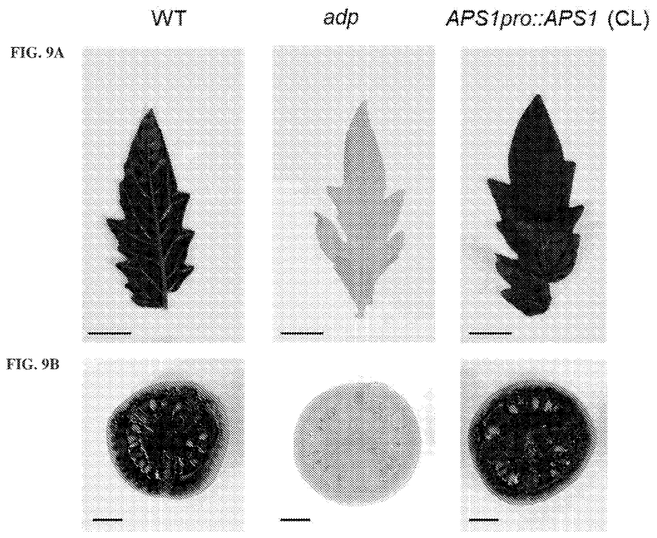

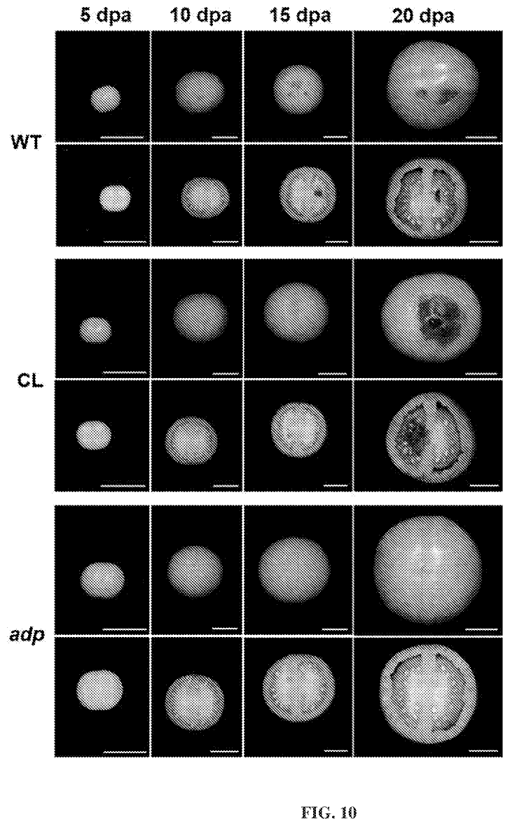

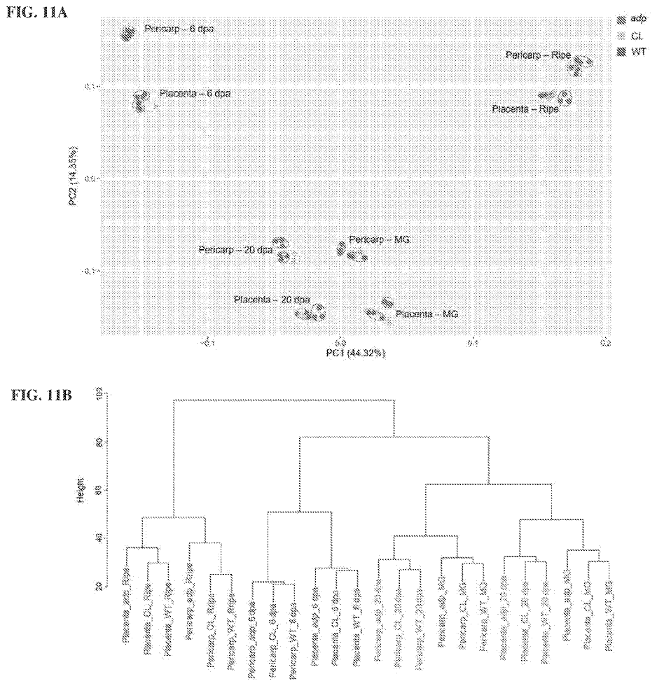

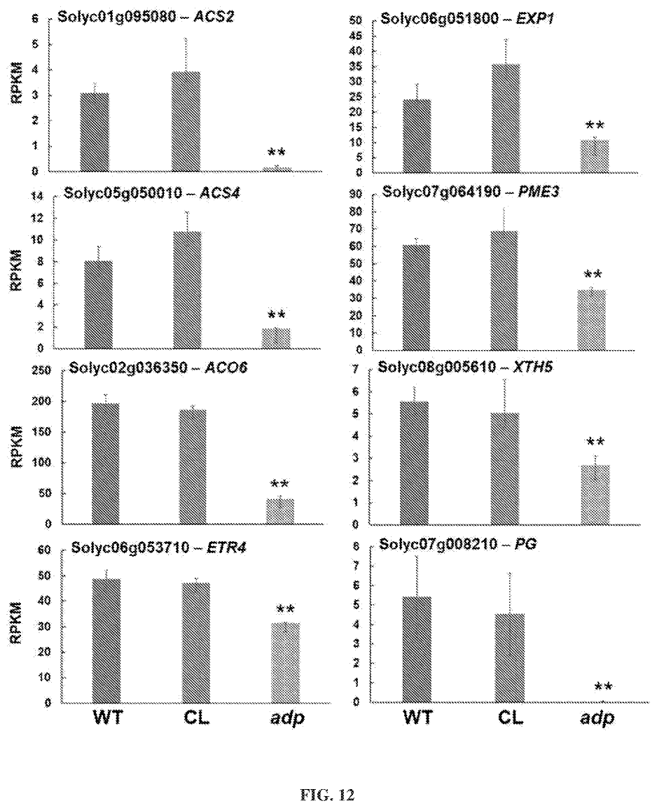

To further confirm that the mutation in APS1 was responsible for the adp phenotype, the starch content was determined by Lugol staining in wild type (WT) and adp tissues. A blue stain after treatment with Lugol reagent indicates the presence of starch. WT plants showed starch accumulation in the root tip, stem cross sections, and leaves, while adp plants showed no Lugol staining (FIG. 1D). Transgenic complementation of adp with a construct comprising the APS1 gene with 2.1 kb upstream and 0.5 kb downstream regions (APS1pro::APS1; CL), generated plants with restored levels of starch (FIGS. 9A-B).

### Example 3—Fruit Development in adpressa

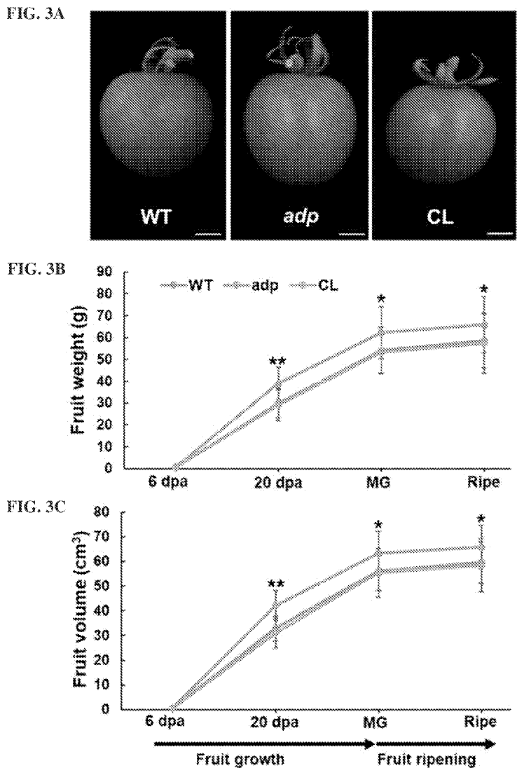

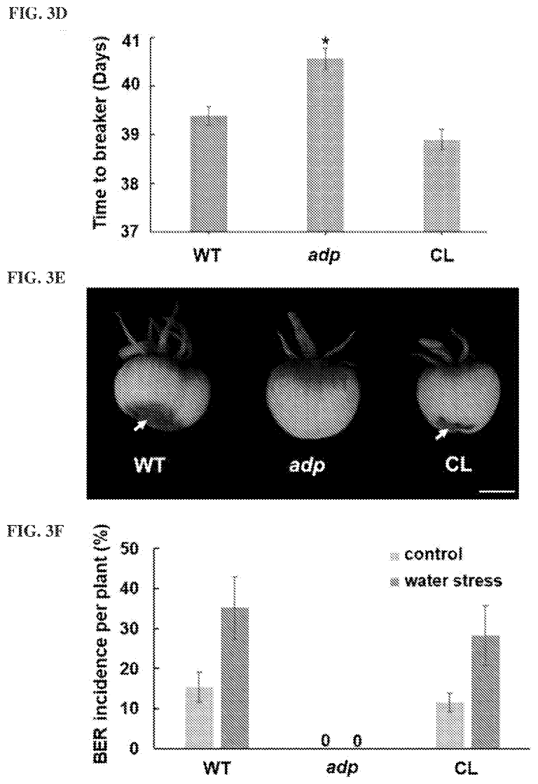

The effect of the adp mutation on the starch content of the fruit at different stages of development by comparing adp to wild type (WT, Ailsa Craig) and CL. In WT and CL fruit, starch accumulated in the pericarp, septum and placenta at the early stages of fruit development, and its level decreased at the onset of ripening (FIGS. 2A-B). The starch content was greater in the internal tissues (e.g., placenta) than in the pericarp (FIG. 2B), where it only accumulated in the inner cell layers (FIG. 2A). Consistent with the starch deficiency observed in the vegetative tissues, no starch staining was observed in the adp fruit throughout development (FIGS. 2A-B). Transgenic complementation of adp also restored levels of starch in the fruit (FIG. 2B and FIGS. 9A-B). To investigate the phenotypic effects of the lack of starch accumulation in the fruit, fruit growth and the time to reach ripening was monitored (FIGS. 3A-D). The overall phenotype of adp fruit was similar to WT, except for a slightly larger size and weight and a minor delay in ripening (~1 day). However, it was observed that adp fruits were remarkably resistant to blossom end rot (BER), a common physiological disorder in tomato that is induced by environmental stress (Topcu et al., “Blossom-End Rot: A Century-Old Problem in Tomato (Solanum lycopersicum L.) and Other Vegetables,” Mol Hort 2:1 (2022), which is hereby incorporated by reference in its entirety), such as drought. To further test BER incidence in adp, WT and adp plants were subjected to water deficit conditions by lowering pot volumetric water content (VWC) to 30% VWC (Nicolas et al., 2022) 5 to 7 days after flowering (DAF), and BER occurrence was assessed. The presence of damaged and necrotic tissue, characteristic of BER, was observed appearing first in the placenta, and later affecting the pericarp, in fruit from the WT and complemented lines (FIG. 10) but not from adp. Evaluation of the BER incidence under control and water stress conditions, showed that adp fruit were never affected by BER (FIGS. 3E-F).

### Example 4—Transcriptional Changes in Adpressa Fruit

To evaluate the effect of the starch deficiency on the fruit transcriptome, an RNA-seq analysis of placenta and pericarp tissues collected from adp, WT and CL fruits at four developmental stages spanning growth and ripening was performed (Table 2).

TABLE 2
Total Mapped Reads per Sample

| Samples | No. reads | % mapped | Samples | No. reads | % mapped |
|---|---|---|---|---|---|
| Pericarp_WT_6dpa_rep1 | 11,632,708 | 94.98 | Pericarp_WT_MG_rep1 | 7,587,173 | 96.68 |
| Pericarp_WT_6dpa_rep2 | 13,624,308 | 95.45 | Pericarp_WT_MG_rep2 | 9,900,335 | 96.10 |
| Pericarp_WT_6dpa_rep3 | 9,603,081 | 95.30 | Pericarp_WT_MG_rep3 | 14,467,428 | 96.53 |
| Placenta_WT_6dpa_rep1 | 9,564,970 | 95.40 | Placenta_WT_MG_rep1 | 12,033,567 | 96.62 |
| Placenta_WT_6dpa_rep2 | 8,718,742 | 94.83 | Placenta_WT_MG_rep2 | 8,432,321 | 95.58 |
| Placenta_WT_6dpa_rep3 | 6,997,935 | 94.89 | Placenta_WT_MG_rep3 | 7,883,133 | 94.27 |
| Pericarp_adp_6dpa_rep1 | 9,284,974 | 95.00 | Pericarp_adp_MG_rep1 | 10,529,521 | 96.37 |
| Pericarp_adp_6dpa_rep2 | 9,183,419 | 95.09 | Pericarp_adp_MG_rep2 | 9,602,416 | 94.04 |
| Pericarp_adp_6dpa_rep3 | 16,088,363 | 95.68 | Pericarp_adp_MG_rep3 | 11,383,976 | 96.07 |
| Placenta_adp_6dpa_rep1 | 11,922,934 | 96.62 | Placenta_adp_MG_rep1 | 8,022,300 | 96.13 |
| Placenta_adp_6dpa_rep2 | 9,294,131 | 94.44 | Placenta_adp_MG_rep2 | 8,724,312 | 94.47 |
| Placenta_adp_6dpa_rep3 | 10,255,737 | 96.03 | Placenta_adp_MG_rep3 | 8,954,166 | 94.35 |
| Pericarp_CL_6dpa_repl | 15,690,078 | 95.97 | Pericarp_CL_MG_rep1 | 8,741,040 | 95.78 |
| Pericarp_CL_6dpa_rep2 | 12,329,030 | 95.44 | Pericarp_CL_MG_rep2 | 7,383,971 | 95.71 |
| Pericarp_CL_6dpa_rep3 | 10,419,427 | 95.59 | Pericarp_CL_MG_rep3 | 6,166,166 | 95.57 |
| Placenta_CL_6dpa_rep1 | 7,750,380 | 94.06 | Placenta_CL_MG_rep1 | 8,922,519 | 95.11 |
| Placenta_CL_6dpa_rep2 | 8,609,206 | 94.67 | Placenta_CL_MG_rep2 | 7,135,769 | 94.61 |
| Placenta_CL_6dpa_rep3 | 9,893,362 | 94.60 | Placenta_CL_MG_rep3 | 8,837,481 | 95.90 |
| Pericarp_WT_20dpa_rep1 | 8,547,565 | 94.59 | Pericarp_WT_Ripe_rep1 | 9,514,218 | 94.24 |
| Pericarp_WT_20dpa_rep2 | 8,726,764 | 95.30 | Pericarp_WT_Ripe_rep2 | 7,511,784 | 80.53 |
| Pericarp_WT_20dpa_rep3 | 9,720,021 | 96.31 | Pericarp_WT_Ripe_rep3 | 8,192,717 | 93.44 |
| Placenta_WT_20dpa_rep1 | 10,095,622 | 94.88 | Placenta_WT_Ripe_rep1 | 17,507,040 | 88.70 |
| Placenta_WT_20dpa_rep2 | 9,844,591 | 95.71 | Placenta_WT_Ripe_rep2 | 7,307,289 | 90.01 |
| Placenta_WT_20dpa_rep3 | 10,979,194 | 94.65 | Placenta_WT_Ripe_rep3 | 12,200,981 | 92.84 |
| Pericarp_adp_20dpa_rep1 | 12,590,146 | 96.23 | Pericarp_adp_Ripe_rep1 | 8,880,889 | 93.96 |
| Pericarp_adp_20dpa_rep2 | 10,498,902 | 95.25 | Pericarp_adp_Ripe_rep2 | 9,005,080 | 94.88 |
| Pericarp_adp_20dpa_rep3 | 6,455,664 | 94.96 | Pericarp_adp_Ripe_rep3 | 8,270,860 | 94.79 |
| Placenta_adp_20dpa_rep1 | 6,246,065 | 94.50 | Placenta_adp_Ripe_rep1 | 11,422,241 | 95.12 |
| Placenta_adp_20dpa_rep2 | 7,320,577 | 95.06 | Placenta_adp_Ripe_rep2 | 7,935,900 | 93.79 |
| Placenta_adp_20dpa_rep3 | 10,614,049 | 94.72 | Placenta_adp_Ripe_rep3 | 8,164,079 | 94.62 |
| Pericarp_CL_20dpa_rep1 | 12,484,732 | 96.05 | Pericarp_CL_Ripe_rep1 | 12,106,508 | 95.27 |
| Pericarp_CL_20dpa_rep2 | 10,241,110 | 95.45 | Pericarp_CL_Ripe_rep2 | 8,629,593 | 95.33 |
| Pericarp_CL_20dpa_rep3 | 11,417,448 | 96.22 | Pericarp_CL_Ripe_rep3 | 9,082,562 | 94.82 |
| Placenta_CL_20dpa_rep1 | 9,505,782 | 96.04 | Placenta_CL_Ripe_rep1 | 7,825,693 | 92.24 |
| Placenta_CL_20dpa_rep2 | 6,496,419 | 95.05 | Placenta_CL_Ripe_rep2 | 7,809,071 | 89.44 |
| Placenta_CL_20dpa_rep3 | 12,753,917 | 95.93 | Placenta_CL_Ripe_rep3 | 8,616,252 | 89.45 |

Mean No. Reads: 9,723,496;
% Mapped: 95

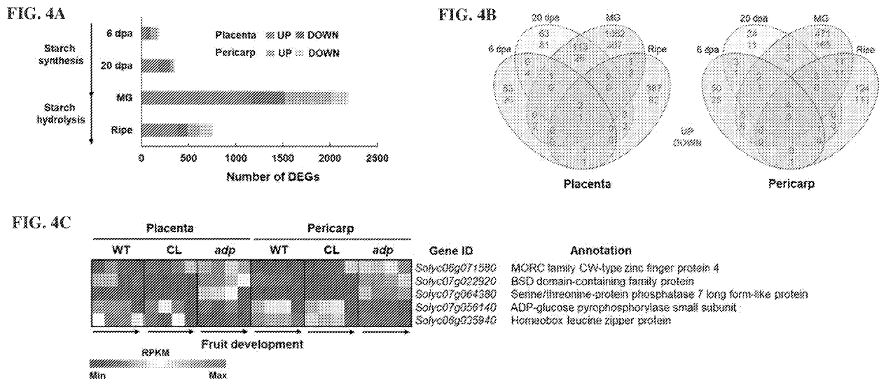

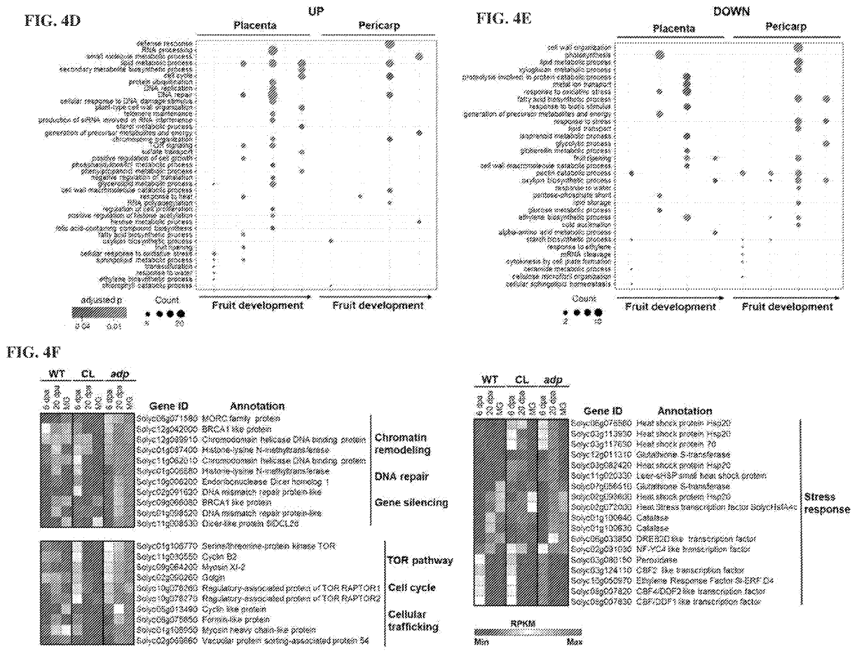

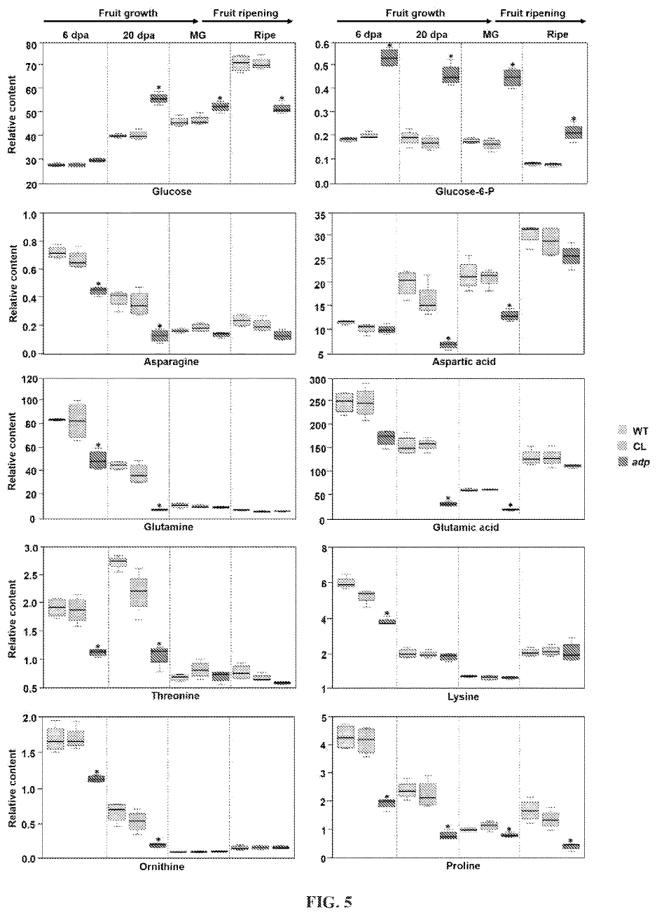

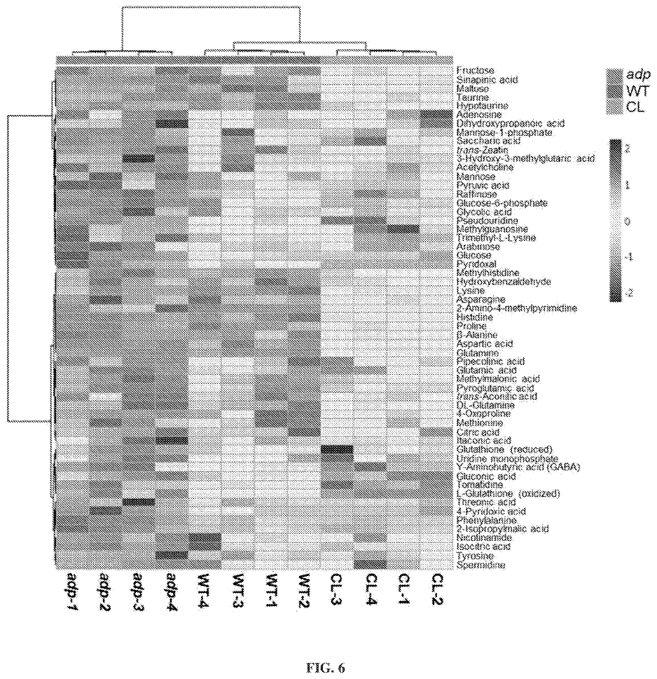

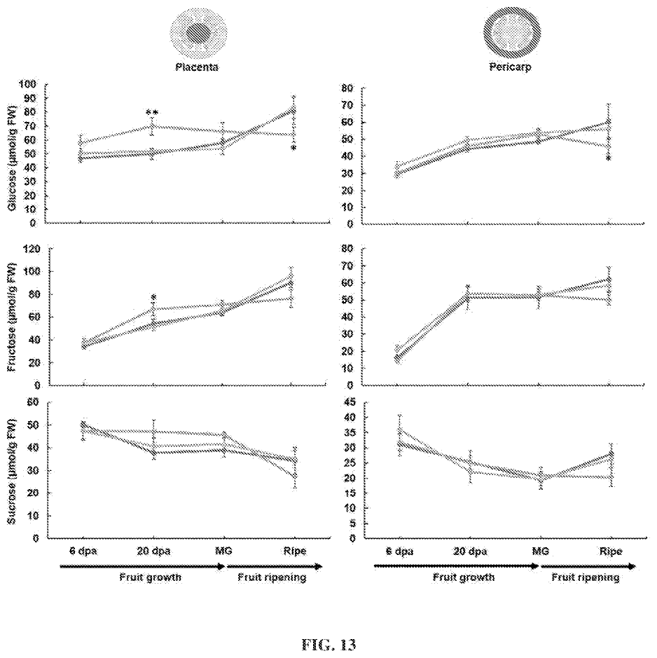

Principal component (PCA) and hierarchical clustering analysis revealed a separation of samples mostly by fruit developmental stage and tissue type (FIGS. 11A-B). A distinct clustering between starch containing (CL and WT) and starchless (adp) fruit was also revealed for all tissues and developmental stages, suggesting major transcriptional changes in adp fruit (FIGS. 11A-B). To increase the confidence of identifying gene expression changes due solely to differences in fruit starch content, differentially expressed genes (DEGs) common to both comparisons: adp vs. CL and adp vs. WT were focused on. The number of DEGs was higher in placenta than in pericarp (FIG. 4A), which correlated with their relative starch content (FIG. 2B). Similarly, the number of DEGs was higher at 20 days post anthesis (dpa), when the placenta starch content peaks, than at 6 dpa at the beginning of the starch accumulation. A higher number of DEGs was identified at the mature green (MG) stage, which marks the end of the starch accumulation phase and is characterized by active starch hydrolysis (FIG. 4A). Overall, upregulated genes were prevalent over downregulated genes in most developmental stages and tissues (FIG. 4A). Except for a significant overlap between 20 dpa and MG developmental stages in placenta, DEGs showed high developmental stage-specificity (FIG. 4B). However, several DEGs were common to pericarp and placenta at different fruit stages (FIG. 4C, Table 3). These included a MICRORCHIDIA (MORC) gene, predicted to be involved in heterochromatin condensation and gene silencing (Koch et al., “MORC Proteins: Novel Players in Plant and Animal Health,” Front Plant Sci 8:1720 (2017), which is hereby incorporated by reference in its entirety), a transcription factor of the BSD (mammalian BTF2-like transcription factors, Drosophila synapse-associated proteins and yeast DOS2-like proteins) family (Fan et al., “A Novel BSD Domain-Containing Transcription Factor Controls Vegetative Growth, Leaf Senescence, and Fruit Quality in Tomato,” J Exp Bot 71:6945-6957 (2020), which is hereby incorporated by reference in its entirety), and a gene encoding a serine/threonine-protein phosphatase, all upregulated in adp. Genes downregulated in adp, at all stages and in all tissues, included a transcription factor of the homeodomain-leucine zipper (HD-Zip) family and APS1. The fact that APS1 transcript levels decreased in adp fruit, suggested that the presence of a non-functional APS1 protein had a negative effect on the expression of this gene (FIG. 4C).

To gain insight into biological processes altered in response to starch deficiency in the fruit, a gene ontology (GO) term enrichment analysis of DEGs downregulated or upregulated in adp fruit at different developmental stages was performed (FIGS. 4D-E, Table 4).

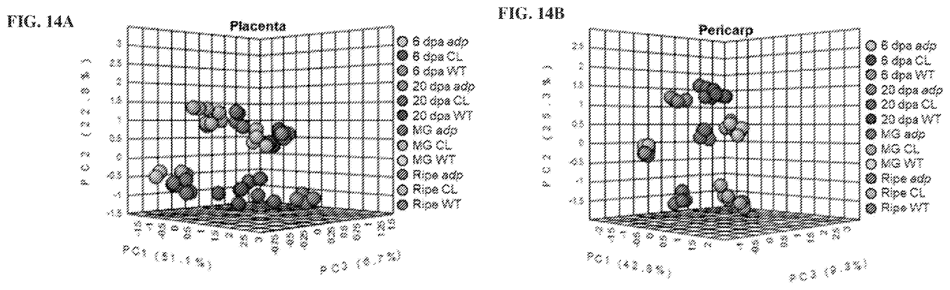

GO terms related to the Target Of Rapamycin (TOR) signaling pathway, regulation of cell growth, DNA repair and epigenetic pathways were predominantly enriched in DEGs upregulated in the placenta at the 20 dpa and MG stages, when starch accumulation peaks in WT fruit (FIG. 4D). Examples of genes in the TOR pathway, cell cycle, chromatin remodeling and gene silencing, that show an increase in expression in the placenta of adp fruit during the starch accumulation period, are shown in FIG. 4F and Table 5. In Table 5, the biological pathway column was manually curated based on the closest homologs in Arabidopsis (TAIR BLAST P best hit). Downregulated DEGs showed a significant enrichment with GO terms related to photosynthesis and glucose related pathways in the placenta at 20 dpa. GO terms related to ripening processes such as cell wall organization, pectin catabolismand ethylene biosynthesis were primarily associated with DEGs downregulated in the adp pericarp at MG (FIG. 4E). These categories comprised ripening-related genes encoding cell wall-modifying enzymes such as expansin, xyloglucan endotransglycosylase/glycosylase, pectin methylestherase, and polygalacturonase, and ethylene-related genes, which all showed reduced expression levels in adp pericarp at the MG stage (FIG. 12).

GO terms associated with stress responses, such as oxidative stress or heat stress response were enriched in DEGs from various developmental stages and tissues. For example, several genes encoding enzymes involved in oxidative stress (e.g., glutathione S-transferases), heat shock proteins and abiotic stress related transcription factors belonging to the C-REPEAT BINDING FACTOR (CBF; Haake et al., “Transcription Factor CBF4 is a Regulator of Drought Adaptation in Arabidopsis,” Plant Physiol 130:639-648 (2002), which is hereby incorporated by reference in its entirety; Medina et al., 2011), HEAT SHOCK FACTOR (HSF; Pérez-Salamó et al., “The Heat Shock Factor A4A Confers Salt Tolerance and is Regulated by Oxidative Stress and the Mitogen-Activated Protein Kinases MPK3 and MPK6,” Plant Physiol 165:319-334 (2014), which is hereby incorporated by reference in its entirety), Ethylene Responsive Factor (ERF; Bahieldin et al., “Ethylene Responsive Transcription Factor ERF109 Retards PCD and Improves Salt Tolerance in Plant,” BMC Plant Biol 16:216 (2016), which is hereby incorporated by reference in its entirety) and Nuclear Factor-Y (NF-Y; Sato et al “Arabidopsis DPB3-1, a DREB2A Interactor, specifically Enhances Heat Stress-Induced Gene Expression by Forming a Heat Stress-Specific Transcriptional Complex with NF-Y Subunits,” Plant Cell 26:4954-4973 (2014), which is hereby incorporated by reference in its entirety) families, were up-regulated in adp during fruit growth (FIG. 4F, Table 5).

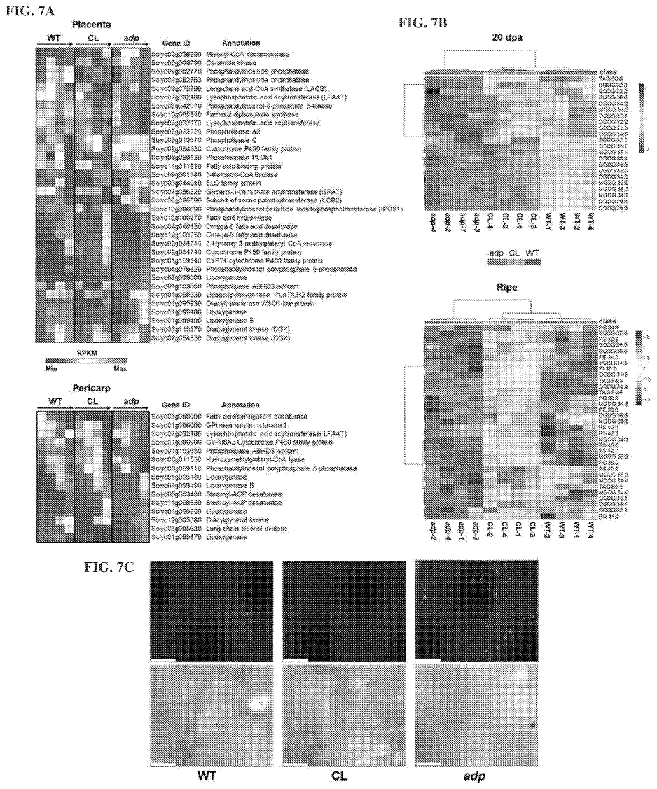

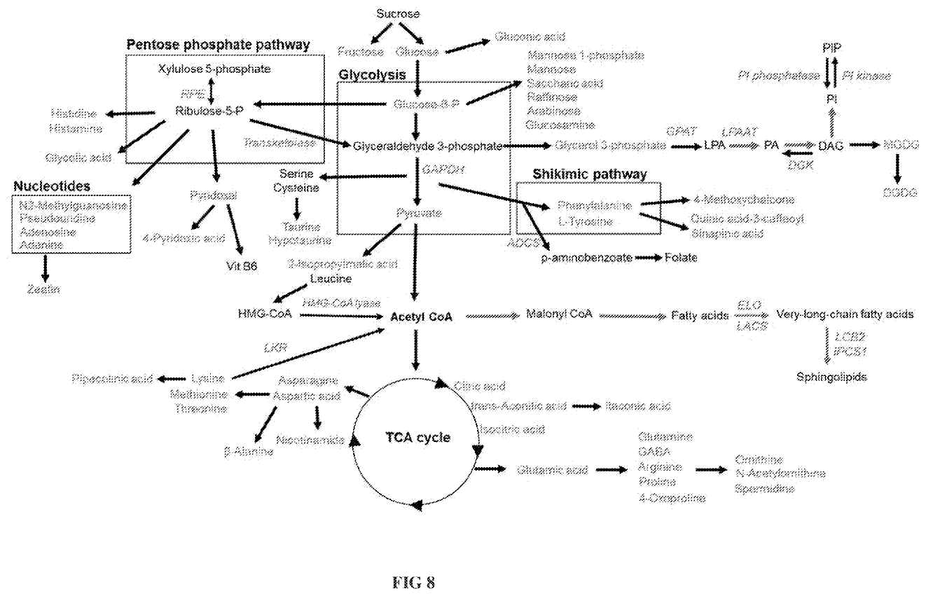

Several GO terms associated with lipid metabolism such as glycerolipid metabolism, sphingolipid biosynthesis, sterol metabolism or fatty acid biosynthesis, were associated to DEG in adp placenta and pericarp (FIG. 4D-E). Analysis of lipid-related gene expression changes (FIG. 7A, Table 5) showed genes involved in sphingolipid metabolism and genes encoding acyltransferases, and lipases implicated in membrane lipid remodeling and in the generation of lipid derived signals, among the DEGs upregulated in the placenta of developing adp fruit, while at the ripe stage upregulated genes comprised those involved in lipid catabolism sterol biosynthesis and genes encoding fatty acid desaturases. In the pericarp, genes associated with sterol biosynthesis, lipid signaling, and phospholipid metabolism increased in expression at the MG and ripe stage. In contrast, several genes encoding lipoxygenases involved in fatty acid oxidation and the oxylipin/jasmonic acid pathway, were downregulated in adp at the ripe stage (FIG. 7A, Table 4).

### Example 5—Metabolite Changes in adpressa Fruit

To determine the effects of starch deficiency on fruit metabolism, changes in metabolite composition in pericarp and placenta from adp fruit compared to WT and CL were analyzed. First, levels of the soluble sugars, glucose, fructose, and sucrose, which are sources of carbon for starch biosynthesis, were quantified using an enzymatic method (Vargas-Ortiz et al., 2013). Surprisingly, despite their close link with starch metabolism, the levels of glucose, fructose, and sucrose were only slightly affected, with an increase in glucose levels (~1.4 x) at 20 dpa and a decrease (~0.8 x) in ripe fruit being the major changes (FIG. 13).

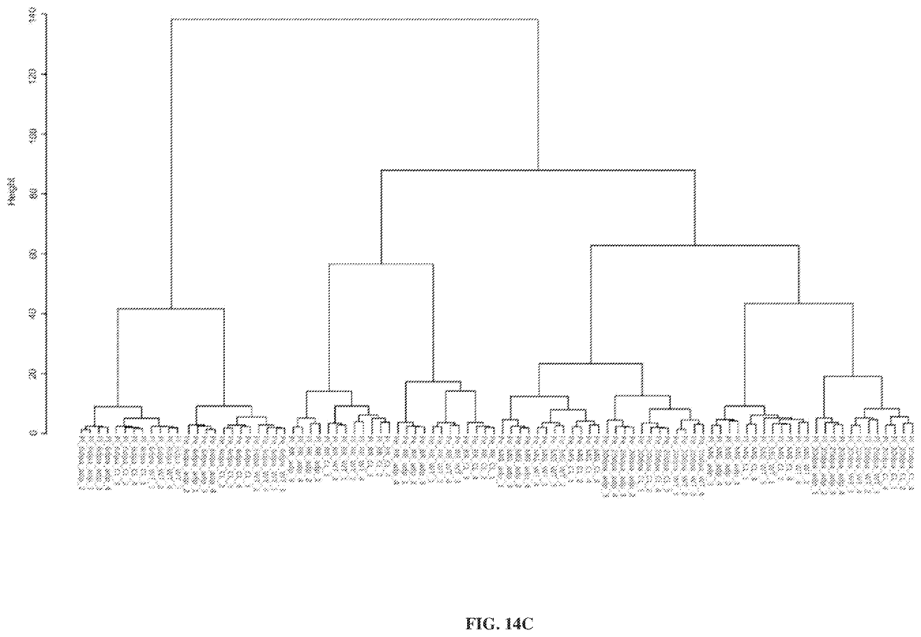

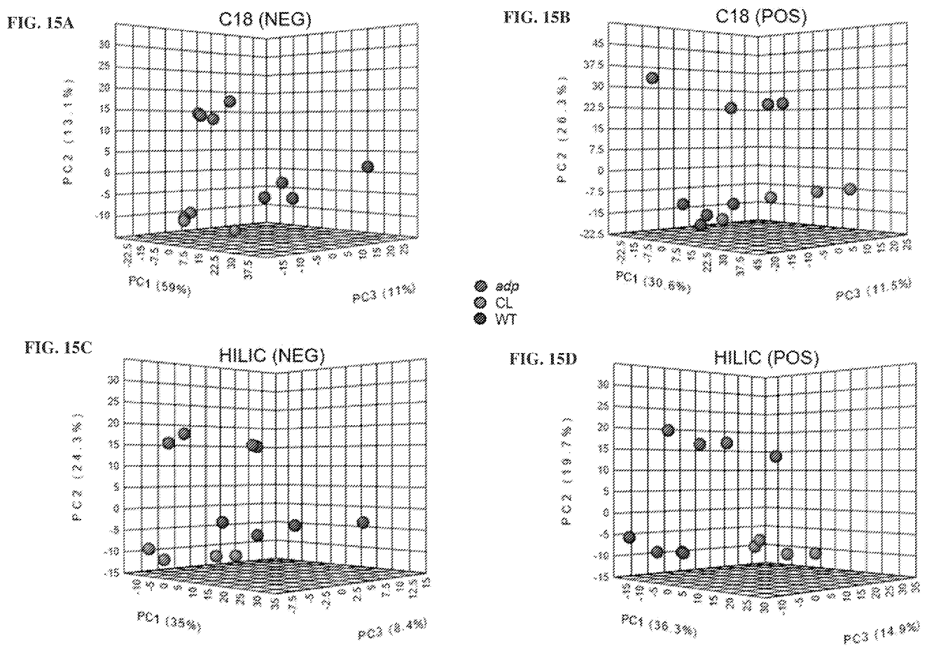

To further investigate the metabolic changes in adp, GC/MS was used to profile primary metabolism compounds (Osorio et al., “Profiling Primary Metabolites of Tomato Fruit with Gas Chromatography/Mass Spectrometry,” Methods Mol Biol 860:101-109 (2012), which is hereby incorporated by reference in its entirety). This analysis showed distinct clusters for each genotype, with replicates for WT and CL tissues showing a closer profile separated from adp (FIGS. 14A-C). Samples separated first according to developmental stages and then by tissue at 6 dpa and the ripe stage but were grouped first by tissue and then by stage at 20 dpa and MG (FIG. 14C). A total of 20 amino acids, 12 sugars, 4 organic acids were quantified, as well as putrescine and phosphoric acid. Significant changes (FDR<0.05) in both, adp vs. WT and adp vs. CL, comparisons were found for 20 metabolites in the placenta, and 15 metabolites in the pericarp, in at least one developmental stage (FIG. 5, Table 6). Bold font indicates significantly lower content in adp vs. both WT and CL fruit (FDR<0.05), and bold italics font indicates significantly higher content in adp vs. both WT and CL fruit (FDR<0.05). These included changes in glucose content, consistent with the sugar enzymatic assay results. Additionally, a significant increase in glucose 6-phosphate (Glc-6-P), a metabolic hub connecting glycolysis and the pentose phosphate pathway, with a key role in energy metabolism, was observed in adp fruit at all developmental stages and tissue. In contrast, adp tissues showed an overall decrease in amino acid content, especially amino acids derived from the tricarboxylic acid (TCA) cycle, such as asparagine, aspartic acid, glutamic acid, glutamine, lysine, threonine, proline and ornithine. The citric acid content was also lower in adp, at the 20 dpa stage, while levels of quinic acid 3-caffeoyl, a phenolic organic acid, were higher at 6 dpa.

To maximize identification of metabolites with altered levels in adp fruit, an untargeted LC-MS/MS analysis using reversed phase and hydrophilic interaction liquid chromatography (HILIC) was also performed, in both positive and negative ionization modes. The placenta of 20 dpa fruit, the tissue that accumulates the highest level of starch in WT and CL lines was analyzed (FIG. 2B). Principal component analysis showed that sample replicates separated according to genotype, with WT and CL samples being closer together than to adp samples (FIGS. 15A-D). 243 metabolites were found showing significant changes (FDR<0.05) in both, adp vs. WT and adp vs. CL comparisons, from which 75 were successfully annotated (Table 7). All listed metabolites showed significant changes in adp vs. both WT and CL (FDR<0.05). This analysis confirmed GC/MS data and showed an increase in soluble sugars (e.g., glucose, Glc-6-P and fructose) and a decrease in the content of several amino acids (e.g., Asn, Asp, Ala, Gln, Glu, Orn, Phe and Pro), and amino acid derivatives (e.g., spermidine and pyroglutamic acid), in adp (FIG. 6). Organic acids derived from the TCA cycle, such as citric acid, trans-aconitic acid and isocitric acid, were also lower in adp. Interestingly, there was also an increase in nucleosides and nucleotides (e.g., pseudouridine, adenine and adenosine) and their derivatives (e.g., zcatin), as well as changes in the levels of stress-related metabolites. The latest included a decrease in pipecolic acid (Wang et al., “Pipecolic Acid Confers Systemic Immunity by Regulating Free Radicals,” Sci Adv 4: eaar4509 (2018), which is hereby incorporated by reference in its entirety) and increases in raffinose, an abiotic stress tolerance mediator (Van den Ende, “Multifunctional Fructans and Raffinose Family Oligosaccharides,” Front Plant Sci 4:247, which is hereby incorporated by reference in its entirety), and various compounds with reactive oxygen species quenching capacity, such as pyridoxal (Czégény et al., “Multiple Roles for Vitamin B6 in Plant Acclimation to UV-B,” Sci Rep 9:1259 (2019), which is hereby incorporated by reference in its entirety) and phenolics (FIG. 6).

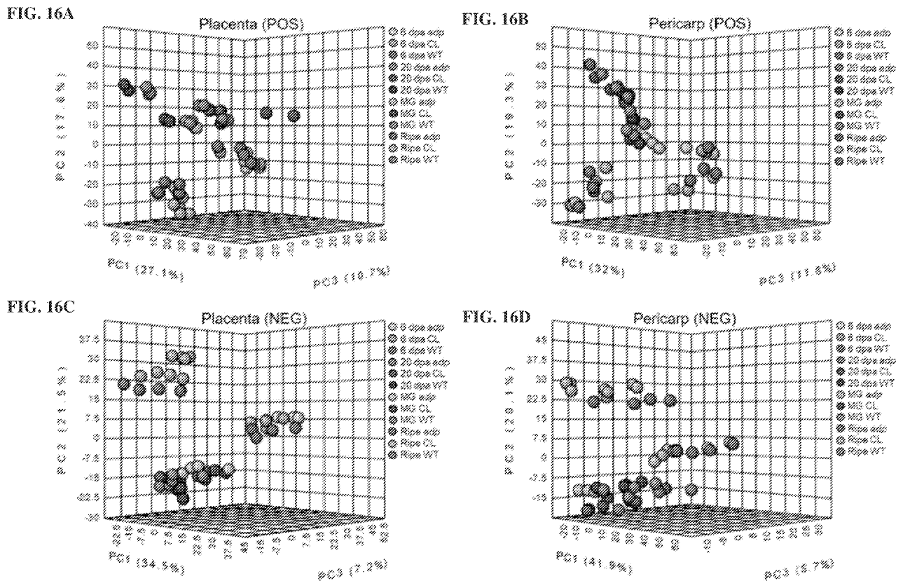

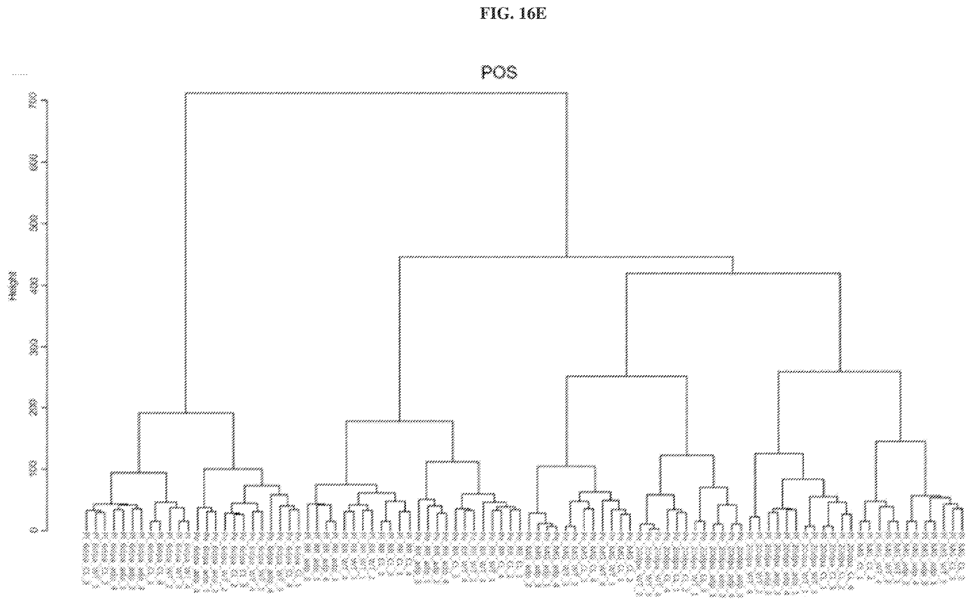

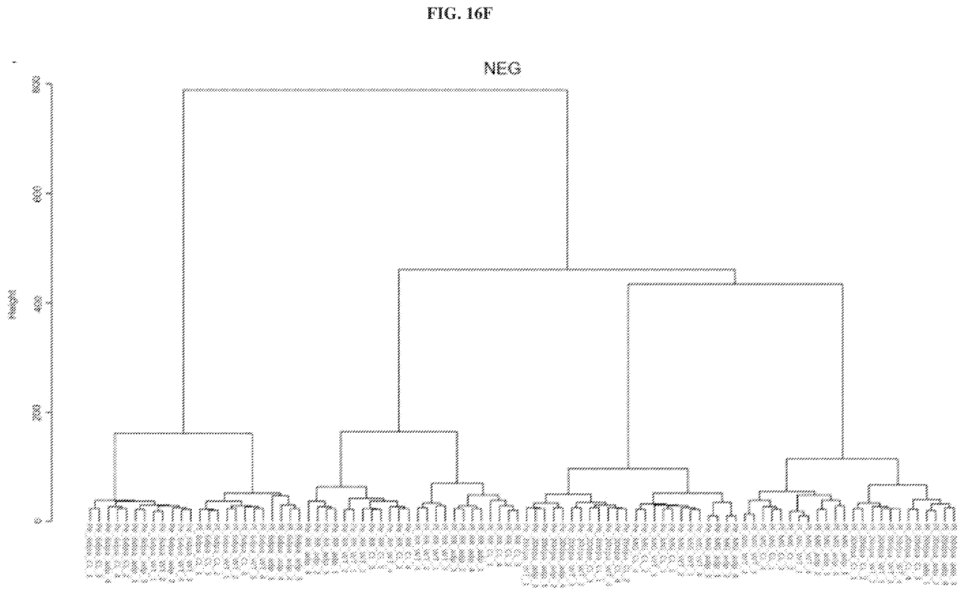

Since the transcriptome data suggested that the adp mutation affected lipid metabolism, a lipidomic analysis comparing adp to the WT and CL fruit tissues was performed. Several classes of glycerolipids were identified and quantified using LC/MS and an in-house library (Lapidot-Cohen et al., “Liquid Chromatography-Mass Spectrometry (LC-MS)-Based Analysis for Lipophilic Compound Profiling in Plants,” Curr Protoc Plant Biol 5: e20109 (2020), which is hereby incorporated by reference in its entirety). PCA and hierarchical clustering analysis of the samples are shown in FIGS. 16A-F. Major differences in the glycerolipid profile of adp tissues were detected, which showed significant changes in 70 and 16 annotated compounds from placenta and pericarp, respectively, in both, adp vs. WT and adp vs. CL, comparisons (Table 8). Italics font indicates significantly higher content in adp vs. both WT and CL fruit (FDR<0.05), and regular font indicates significantly lower content in adp vs. both WT and CL fruit (FDR<0.05). Notably, an important decrease in the chloroplast membrane lipids, monogalactosyldiacylglycerols (MGDGs) and digalactosyldiacylglycerols (DGDGs), was observed in the placenta of adp fruit at the 20 dpa stage, the peak of starch accumulation in WT and CL lines (FIG. 7B). At the ripe stage, significant differences involved a general decrease in the degree of desaturation of MGDGs, DGDGs and sulfoquinovosyldiacylglycerols (SQDG), and changes in levels and desaturation of some phospholipids (FIG. 7B).

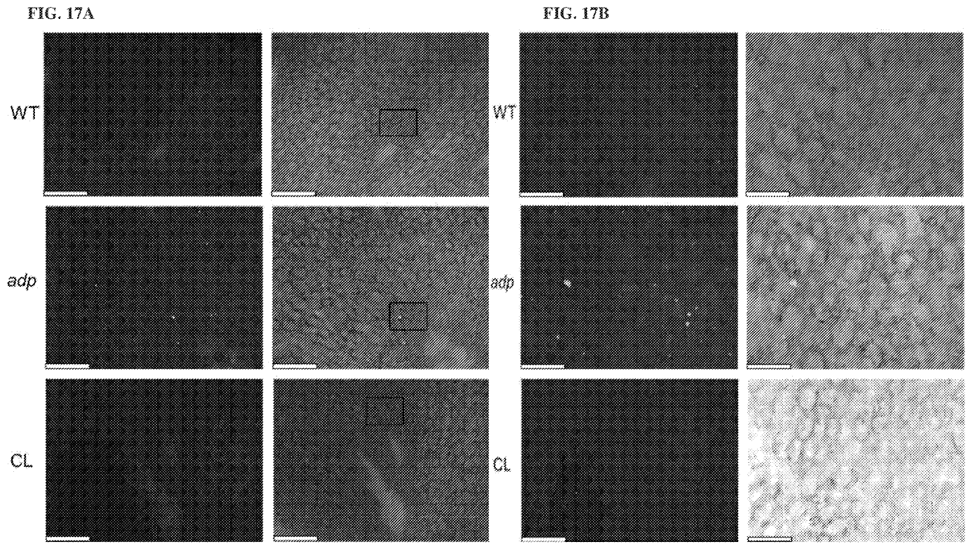

Mutants in starch biosynthesis have been shown to redirect carbon to lipid synthesis which accumulate as triacylglycerols (TAG) in lipid droplets (LDs) (Yu et al., “Starch Deficiency Enhances Lipid Biosynthesis and Turnover in Leaves,” Plant Physiol 178:118-129 (2018); Xu et al., “Upregulated Lipid Biosynthesis at the Expense of Starch Production in Potato (Solanum Tuberosum) Vegetative Tissues Via Simultaneous Downregulation of ADP-Glucose Pyrophosphorylasc and Sugar Dependent1 Expressions,” Front Plant Sci. 10:1444 (2019); each of which is hereby incorporated by reference in its entirety). Further indication of changes in lipid metabolism in adp was obtained by the visualization of lipid droplets in 20 dpa adp fruit. Fruit sections stained with the fluorophore BODIPY 505/515, which primarily stains neutral storage LDs (Govender et al., “BODIPY Staining, an Alternative to the Nile Red Fluorescence Method for the Evaluation of Intracellular Lipids in Microalgae,” Bioresour Technol 114:507-511 (2012), which is hereby incorporated by reference in its entirety), revealed an accumulation of LDs in the adp septum (FIG. 7C), and to a lesser extent in the placenta (FIGS. 17A-B), while they were not detected in WT and CL lines. This suggests a buildup of lipids stored as TAGs as a consequence of the starch deficiency in adp.

A summary of the metabolic changes in the placenta of growing adp fruit as a result of the starch deficiency is shown in FIG. 8.

### Example 6—Discussion of Examples 2-5

Tomato fruit development is accompanied by a transient accumulation of starch which is thought to act as a carbon reservoir to support sink strength, buffer fluctuations in energy supply, and contribute to sugar accumulation at ripening (Ruan et al., “Molecular Regulation of Seed and Fruit Set,” Trends Plant Sci 17:656-665 (2012); Colombié et al., “Respiration Climacteric in Tomato Fruits Elucidated by Constraint-Based Modelling,” New Phytol 213:1726-1739 (2017); Roch et al., “Fruit Salad in the Lab: Comparing Botanical Species to Help Deciphering Fruit Primary Metabolism,” Front Plant Sci 10:836 (2019), each of which is hereby incorporated by reference in its entirety). However, despite these suggested key roles of starch, the impact that transitory starch accumulation has on transcriptional and metabolic pathways during fruit development is still unknown. Starch biosynthesis mutants in Arabidopsis (Streb and Zeeman, “Starch Metabolism in Arabidopsis,” The Arabidopsis Book 10: e0160 (2012), which is hereby incorporated by reference in its entirety) and grain crop species (Baysal et al., “Inactivation of Rice Starch Branching Enzyme IIb Triggers Broad and Unexpected Changes in Metabolism by Transcriptional Reprogramming,” PNAS 117:26503-26512 (2020); Finegan et al., “Genetic Perturbation of the Starch Biosynthesis in Maize Endosperm Reveals Sugar-Responsive Gene Networks,” Front Plant Sci 12:800326 (2022), each of which is hereby incorporated by reference in its entirety) have provided insight on the molecular and physiological effects of perturbing starch metabolism, however, starch-deficient mutants have not been characterized in fleshy fruit species to datc.

The identification of the adp mutation in tomato was investigated and found to be a consequence of a deletion in the AGPase small subunit, which renders adp plants unable to synthesize starch. The effect of starch deficiency on fruit growth and development was investigated, and the transcriptional and metabolic responses that are triggered in the fruit by the lack of starch accumulation were analyzed.

Surprisingly, the lack of starch biosynthesis did not cause major changes in fruit development except for an increase in size and weight (FIG. 3). One of the significant metabolic effects of the starch deficiency was the higher levels of soluble sugars, mainly glucose (FIG. 5 and FIG. 13) which can act as a signaling molecule and osmolyte to stimulate cell division and expansion, respectively (Wang and Ruan, “Regulation of Cell Division and Expansion by Sugar and Auxin Signaling,” Front Plant Sci 4:163 (2013); Lastdrager et al., “Sugar Signals and the Control of Plant Growth and Development,” J Exp Bot 65:799-807 (2014), each of which is hereby incorporated by reference in its entirety). Therefore, the higher soluble sugar content could be stimulating fruit growth in adp fruit.

Although there were only minor changes in fruit development, the lack of starch synthesis generated an important transcriptional remodeling in adp fruit. This was especially significant in the tissue (placenta) and developmental stages (20 dpa, MG) corresponding to higher levels of starch accumulation in WT fruit (FIGS. 2A-B and FIG. 4A). DEGs in adp were enriched with genes involved in small RNA biogenesis and chromatin remodeling, suggesting that changes in adp fruit may be the result of post-transcriptional and/or epigenetic regulation. A member of the MORC protein family, involved in chromatin remodeling (Koch et al., “MORC Proteins: Novel Players in Plant and Animal Health,” Front Plant Sci 8:1720 (2017); Xue et al., “Arabidopsis MORC Proteins Function in the Efficient Establishment of RNA Directed DNA Methylation,” Nat Commun 12:4292 (2021), each of which is hereby incorporated by reference in its entirety), was strongly upregulated in adp tissues at all developmental stages (FIG. 4C). Arabidopsis MORC mutants show modified photosynthesis efficiency accompanied by stunted growth and increased starch accumulation (van Tol et al., “An Arabidopsis Mutant with High Operating Efficiency of Photosystem II and Low Chlorophyll Fluorescence,” Sci Rep 7:3314 (2017), which is hereby incorporated by reference in its entirety). This is consistent with the data from this study showing that, in reverse, starch deficiency associates with greater MORC expression and fruit growth. The fact that starch content may be inversely correlated with MORC expression and cell growth suggests a link between sugar metabolism/signaling and MORC-mediated transcriptional gene silencing.

The transcriptional response to starch deficiency also involved a significant increase in expression of TOR signaling genes, including the TOR kinase and its associated proteins (FIG. 4F). The TOR pathway is involved in sugar sensing and is a key regulator integrating environmental and nutrient signals to control growth and development (Ryabova et al., “Target of Rapamycin Kinase: Central Regulatory Hub for Plant Growth and Metabolism,” J Exp Bot 70:2211-2216 (2019); McCready et al., “The Importance of TOR Kinase in Plant Development,” Front Plant Sci 11:16 (2020), each of which is hereby incorporated by reference in its entirety). Glucose upregulates the TOR pathway and glucose-TOR signaling dictates transcriptional reprogramming involving central and secondary metabolism, cell cycle, transcription, signaling, transport and protein folding (Xiong et al., “Glucose-TOR Signalling Reprograms the Transcriptome and Activates Meristems,” Nature 496:181-186 (2013), which is hereby incorporated by reference in its entirety). The repression of TOR expression in Arabidopsis caused an increase in starch content (Caldana et al., “Systemic Analysis of Inducible Target of Rapamycin Mutants Reveal a General Metabolic Switch Controlling Growth in Arabidopsis thaliana,” Plant J 73:897-909 (2013), which is hereby incorporated by reference in its entirety), and it has recently been shown that, in algae, starch deficiency upregulates TOR activity (Mallén-Ponce et al., “Photosynthetic Assimilation of CO2 Regulates TOR Activity,” PNAS 119: e2115261119 (2022), which is hereby incorporated by reference in its entirety), suggesting a link between the TOR pathway and starch metabolism. These results indicate that part of the transcriptional changes in adp might be mediated by the glucose-TOR signaling pathway and triggered by the increase in glucose content during fruit growth (FIG. 5; FIG. 13; Table 6). Significant gene expression changes in adp also included upregulation of genes encoding cell cycle related proteins (e.g. cyclins, DNA repair proteins, myosins, formins, actin-cross-linking protein; FIG. 4, Table 4). The potential stimulation of cell cycle in adp is also supported by an increase in trans-zeatin (FIG. 6), the prevalent plant cytokinin, in the placenta of adp fruit. Consistent with this observation, higher levels of cytokinin have been found in AGPase-deficient pea embryos (Weigelt et al., “ADP-Glucose Pyrophosphorylase-Deficient Pea Embryos Reveal Specific Transcriptional and Metabolic Changes of Carbon-Nitrogen Metabolism and Stress Responses,” Plant Physiol 149:395-411 (2009), which is hereby incorporated by reference in its entirety). These results suggest a positive regulation of the cell cycle, growth promotion, and stimulation of cytokinin-mediated cell proliferation, possibly through an activation of the TOR pathway in adp.

Ripening-related transcriptional changes in adp fruit which occurred mainly in the pericarp and included downregulation of genes involved in ethylene biosynthesis and of multiple cell wall modifying genes was also observed (FIG. 12) associated with ripening (Quinet et al., “Tomato Fruit Development and Metabolism,” Front Plant Sci. 10:1554 (2019), which is hereby incorporated by reference in its entirety). These changes, together with the slight delay of adp fruit to reach the ripening phase, further suggest that starch hydrolysis at the end of the growth period may influence the onset of ripening (Colombié et al., “Respiration Climacteric in Tomato Fruits Elucidated by Constraint-Based Modelling,” New Phytol 213:1726-1739 (2017), which is hereby incorporated by reference in its entirety). However, the absence of major changes in fruit ripening capability in adp indicates that starch hydrolysis is not required for the initiation and completion of the ripening process in tomato.

Changes in the metabolite composition of adp fruit indicate that the disruption of starch synthesis causes an important redirection of metabolic flux (FIG. 8, FIGS. 14A-C, and FIGS. 15A-D). During fruit growth, higher levels of soluble sugars in adp were observed together with an increase in glucose-6-P, and a decrease in amino acids and TCA cycle organic acids (FIG. 5 and FIG. 6). This could be caused by the redirection of the acetyl CoA pool towards fatty acid biosynthesis, and/or to an elevated metabolism of TCA cycle intermediates, and amino acids, to support the higher fruit growth in adp. The overall decrease in amino acids in adp, contrasts with the increase in amino acid levels observed in rice starch mutants (Baysal et al., “Inactivation of Rice Starch Branching Enzyme IIb Triggers Broad and Unexpected Changes in Metabolism by Transcriptional Reprogramming,” PNAS 117:26503-26512 (2020), which is hereby incorporated by reference in its entirety). In pea embryos, the reduction in AGPase expression caused a decrease in levels of certain amino acids, while an increase in others (Weigelt et al., “ADP-Glucose Pyrophosphorylase-Deficient Pea Embryos Reveal Specific Transcriptional and Metabolic Changes of Carbon-Nitrogen Metabolism and Stress Responses,” Plant Physiol 149:395-411 (2009), which is hereby incorporated by reference in its entirety). Therefore, it is likely that different species, tissues, organs, and developmental stages differ in how carbon flux is redirected, and which metabolite pools are affected in absence of starch synthesis. These results also suggest that metabolic adjustments in adp involve channeling the excess of sugars into specific branches fueled by the glycolytic pathway. For example, the downregulation of transketolase and ribulose-phosphate 3-epimerase observed in adp could result in glucose-6-P and ribulose-5-P being directed towards nucleotide and nucleoside synthesis, causing an increase in the levels of these metabolites (FIG. 6 and FIG. 8).

Disruption of starch synthesis in adp also resulted in the remodeling of the lipid metabolism. Metabolomics data highlighted substantial changes in the profile of glycerolipids, the main structural components of cellular membranes (Yu et al., “Mechanisms and Functions of Membrane Lipid Remodeling in Plants,” Plant J 107:37-53 (2021), which is hereby incorporated by reference in its entirety) which in the growing fruit (20 dpa) was reflected in a strong decrease in MGDG and DGDG and, in ripe tissue, by a decrease in the level of membrane lipid unsaturation (FIG. 7B), The lower expression of a glycerol 3-phosphate acyltransferase and/or the potential redirection of glycerolipid biosynthesis towards phosphatidic acid (PA), due to increased expression of LPAAT and DGK encoding genes (FIG. 7A, FIG. 8), could contribute to the decrease in MGDG and DGDG. This transcriptome data also suggests upregulation of the biosynthesis of sphingolipids, long chain fatty acids, and sterols in adp (FIG. 7A). Whether this translates into elevated levels of these compounds will need to be validated using a targeted metabolite analysis approach. It was also observed an enhanced accumulation of LD in the septum of adp fruit (FIG. 7C, Supplemental FIG. S9), indicative of increased TGA levels. This underscores the redirection of carbon skeletons towards fatty acid and lipid biosynthesis due to a disruption in starch biosynthesis. Although both septum and placenta accumulate starch in WT fruit (FIG. 2A), LDs were present preferentially in the septum, suggesting tissue specificity in the capacity for TGA accumulation (Lu et al., “Spatial Analysis of Lipid Metabolites and Expressed Genes Reveals Tissue-Specific Heterogeneity of Lipid Metabolism in High- and Low-Oil Brassica Napus L. Seeds,” Plant J 94:915-932 (2018), which is hereby incorporated by reference in its entirety).

A striking phenotypic trait of adp fruit was their total lack of BER symptoms even when subjected to water stress, which is a common trigger of BER (FIG. 3E-F; FIG. 2A-B). In tomato, as well as other fruit crops (e.g., peppers, squash, cucumber, and melon), environmental stresses cause BER, characterized by water-soaked tissues and necrosis. Calcium deficiency (Ho and White, “A Cellular Hypothesis for the Induction of Blossom-End Rot in Tomato Fruit,” Ann Bot 95:571-581 (2005); De Freitas et al., “Role of Pectin Methylesterases in Cellular Calcium Distribution and Blossom-End Rot Development in Tomato Fruit,” Plant J 71:824-835 (2012), each of which is hereby incorporated by reference in its entirety), and increased ROS production (Saure, “Why Calcium Deficiency is not the Cause of Blossom-End Rot in Tomato and Pepper Fruit-A Reappraisal,” Sci Hortic 174:151-154 (2014); De Freitas et al., “Role of Pectin Methylesterases in Cellular Calcium Distribution and Blossom-End Rot Development in Tomato Fruit,” Plant J 71:824-835 (2012); Hagassou et al., “Blossom End-Rot in Tomato (Solanum lycopersicum L.): A Multi-Disciplinary Overview of Inducing Factors and Control Strategies,” Scientia Horticulturae 249:49-58 (2019), each of which is hereby incorporated by reference in its entirety) have been suggested as the origins of BER. However, the molecular mechanisms underlying this physiological disorder are not understood and still a matter of debate (Topcu et al., “Blossom-End Rot: A Century-Old Problem in Tomato (Solanum lycopersicum L.) and Other Vegetables,” Mol Hort 2:1 (2022); Hagassou et al., “Blossom End-Rot in Tomato (Solanum lycopersicum L.): A Multi-Disciplinary Overview of Inducing Factors and Control Strategies,” Scientia Horticulturae 249:49-58 (2019), each of which is hereby incorporated by reference in its entirety). A link with carbohydrate metabolism has also been suggested after observations of higher levels of symplastic starch in pepper fruits affected by BER (Turhan et al., “Blossom-End Rot is Associated with Impairment of Sugar Metabolism and Growth of Pepper (Capsicum Annuum L.) Fruits,” J Hortic Sci Biotechnol 81:921-927 (2006), which is hereby incorporated by reference in its entirety). The data disclosed herein suggest that sugar metabolism plays a central role in BER resistance. In adp this resistance could potentially be an indirect consequence of TOR-mediated activation of cell growth and survival (Gonzalez and Rallis, “The TOR Signaling Pathway in Spatial and Temporal Control of Cell Size and Growth,” Front Cell and Dev Biol 5:61 (2017), which is hereby incorporated by reference in its entirety) (FIG. 4F, Table 5), together with the promotion of abiotic stress responses and stress adaptation (Fu et al., “Target of Rapamycin Signaling in Plant Stress Responses,” Plant Physiol 182:1613-1623 (2020); Haq et al., “Roles of TOR Signaling in Nutrient Deprivation and Abiotic Stress,” J Plant Physiol 274:153716 (2022), each of which is hereby incorporated by reference in its entirety). Changes in adp fruit included an upregulation of genes encoding enzymes involved in suppressing oxidative damage, and transcription factors involved in the adaptive response to various abiotic stresses (FIG. 4F). Similarly, metabolites involved in tolerance to stress and ROS quenching, such as raffinose, phenolics or pyridoxal, showed increased levels in adp (FIG. 6, Table 6). In addition to an up-regulation of stress-related pathways, the lipid composition changes observed in adp could also lead to a better protection of fruit tissues from stress, therefore contributing to the BER resistant phenotype. Lipid remodeling of cell membranes is a critical mechanism countering loss of membrane integrity and cell death during stress responses (Yu et al., “Mechanisms and Functions of Membrane Lipid Remodeling in Plants,” Plant J 107:37-53 (2021), which is hereby incorporated by reference in its entirety). The lipid-related changes in adp mimic lipid-remodeling responses triggered by abiotic stresses, such as cold, heat or drought (FIG. 7B-C). These include a reduction in MGDG levels, increase in TGA accumulation and a decrease in the degree of membrane lipid unsaturation (de Vries. and Ischebeck, “Ties Between Stress and Lipid Droplets Pre-Date Seeds,” Trends Plant Sci 25:1203-1214 (2020); Yu et al., “Mechanisms and Functions of Membrane Lipid Remodeling in Plants,” Plant J 107:37-53 (2021), each of which is hereby incorporated by reference in its entirety). Both TGA accumulation and a decrease in the degree of membrane lipid unsaturation have been associated with heat stress in tomato fruit (Almeida et al., “A Transcriptomic, Metabolomic and Cellular Approach to the Physiological Adaptation of Tomato Fruit to High Temperature,” Plant, Cell & Environ 44:2211-2229 (2021), which is hereby incorporated by reference in its entirety). Furthermore, genes involved in PA formation and sphingolipid biosynthesis (FIG. 7A, Table 5), both with a role during abiotic stress responses (Yao and Xue, “Phosphatidic Acid Plays Key Roles Regulating Plant Development and Stress Responses,” J Integr Plant Biol 60:851-863 (2018); Huby et al., “Sphingolipids: Towards an Integrated View of Metabolism During the Plant Stress Response,” New Phytol 225:659-670 (2020); each of which is hereby incorporated by reference in its entirety), were expressed at higher levels in adp growing fruit.

In summary, these results indicate that the starch deficiency in adp causes major transcriptional and metabolic adjustments affecting core metabolic pathways (FIG. 8). The lack of carbon flux into starch generates a greater abundance of sugars, increased carbon reallocation towards lipid biosynthesis, and modification of gene expression that leads to enhanced growth and stress-protection responses. This study offers insights into the significance of starch metabolism for fruit growth and ripening and highlights the link between sugar metabolism and resistance to cellular damage in fruit tissues, which can open new paths to improve fruit yield and quality under abiotic stress conditions.

Accession Numbers. The accession number of the genes mentioned in Examples 1-6 can be found in Tables 9-12. Sequence data can be found in the Solanaceae Genomics Network (SGN) database (solgenomics.net/organism/Solanum_lycopersicum/genome; ITAG Release 3.2).

TABLE 3
List of DEGs Shared Between Pericarp and Placenta at Least in One Developmental Stage

| | Identifier | | UP/ | Developmental | |
|---|---|---|---|---|---|
| Locus | (ITAG 3.2) | Annotation (ITAG 3.2) | DOWN | stages | |
| Solyc07g022920 | Solyc07g022920.3.1 | BSD domain-containing family protein (AHRD V3.3 *** B9IBP6_POPTR) | UP | 6 dpa, 20 dpa, | |
| | | | | MG, Ripe | |
| Solyc06g071580 | Solyc06g071580.3.1 | MORC family CW-type zinc finger protein 4 (AHRD V3.3 *** | UP | 6 dpa, 20 dpa, | |
| | | A0A0B0PTV2_GOSAR) | | MG, Ripe | |
| Solyc07g064380 | Solyc07g064380.3.1 | serine/threonine-protein phosphatase 7 long form-like protein (AHRD V3.3 *-* | UP | 6 dpa, 20 dpa, | |
| | | AT1G32120.1) | | MG | |
| Solyc07g056140 | Solyc07g056140.3.1 | ADP-glucose pyrophosphorylase small subunit | DOWN | 6 dpa, 20 dpa, | |
| | | | | MG | |
| Solyc06g035940 | Solyc06g035940.3.1 | Homeobox leucine zipper protein (AHRD V3.3 *** A0A072TNH2_MEDTR) | DOWN | 6 dpa, 20 dpa | |
| Solyc06g033850 | Solyc06g033850.3.1 | Dehydration responsive element-binding factor protein (AHRD V3.3 --* | UP | 20 dpa, MG | |
| | | A0A0K0K9Y3_ELECO) | | | |
| Solyc08g016440 | Solyc08g016440.3.1 | Polynucleotidyl transferase, ribonuclease H-like superfamily protein (AHRD | UP | 6 | dpa |
| | | V3.3 --* AT2G13980.1) | | | |
| Solyc02g091990 | Solyc02g091990.3.1 | 1-aminocyclopropane-1-carboxylate synthase 3 | UP | 6 | dpa |
| Solyc03g058910 | Solyc03g058910.3.1 | Pectate lyase (AHRD V3.3 *** M1A3P9_SOLTU) | DOWN | 6 | dpa |
| Solyc12g087830 | Solyc12g087830.2.1 | MADS-box transcription factor (AHRD V3.3 *** FIT119_9ERIC) | DOWN | 6 | dpa |
| Solyc02g093600 | Solyc02g093600.3.1 | Class I heat shock protein (AHRD V3.3 *** F4YBC5_SOLNI) | UP | 20 | dpa |
| Solyc06g076560 | Solyc06g076560.2.1 | 17.6 kD class I small heat shock protein | UP | 20 | dpa |
| Solyc03g082420 | Solyc03g082420.3.1 | Heat shock protein (AHRD V3.3 *** A9QVH3_9FABA) | UP | 20 | dpa |
| Solyc03g113930 | Solyc03g113930.2.1 | Heat-shock protein, putative (AHRD V3.3 *** B9S5K5_RICCO) | UP | 20 | dpa |
| Solyc07g056510 | Solyc07g056510.3.1 | Glutathione S-transferase (AHRD V1 **** D3Y4H6_9ROSI) | UP | 20 | dpa |
| Solyc02g071820 | Solyc02g071820.3.1 | Receptor-like protein kinase (AHRD V3.3 *** B9I1R1_POPTR) | UP | 20 | dpa |
| Solyc11g020330 | Solyc11g020330.1.1 | leer-sHSP small heat shock protein | UP | 20 | dpa |
| Solyc06g009190 | Solyc06g009190.3.1 | Pectinesterase (AHRD V3.3 *** K4C3U9_SOLLC) | DOWN | 20 | dpa |
| Solyc04g064690 | Solyc04g064690.3.1 | Peroxidase (AHRD V3.3 *** K4BT60_SOLLC) | DOWN | 20 | dpa |

| | | | | |
|---|---|---|---|---|
| Solyc02g084280 | Solyc02g084280.3.1 | Nuclear pore complex protein Nup98b (AHRD V3.3 *-* W6JLG1_NICBE) | UP | MG |
| Solyc10g076260 | Solyc10g076260.2.1 | Regulatory-associated protein of TOR protein (AHRD V3.3 *-* | UP | MG |
| | | G7KW32_MEDTR) | | |
| Solyc07g032180 | Solyc07g032180.3.1 | Abhydrolase domain-containing protein 4 (AHRD V3.3 *** | UP | MG |
| | | A0A0B2SN17_GLYSO) | | |
| Solyc07g025490 | Solyc07g025490.2.1 | OSBP(oxysterol binding protein)-related protein 4B (AHRD V3.3 --* | UP | MG |
| | | AT4G25850.3) | | |
| Solyc05g056040 | Solyc05g056040.3.1 | Auxin Response Factor 24 | UP | MG |
| Solyc11g020960 | Solyc11g020960.2.1 | Proteinase inhibitor II (AHRD V3.3 *-* B3FOC1_TOBAC) | UP | MG |
| Solyc06g011530 | Solyc06g011530.3.1 | Hydroxymethylglutaryl-CoA lyase (AHRD V3.3 *-* W9QRW1_9ROSA) | UP | MG |
| Solyc09g066080 | Solyc09g066080.2.1 | Protein BREAST CANCER SUSCEPTIBILITY 1-like protein (AHRD V3.3 *-* | UP | MG |
| | | W9R323_9ROSA) | | |
| Solyc09g010320 | Solyc09g010320.3.1 | Mitochondrial fission protein ELM1 (AHRD V3.3 *** A0A199UV44_ANACO) | UP | MG |
| Solyc03g059150 | Solyc03g059150.3.1 | Ion channel DMI1 (AHRD V3.3 * DMI1_MEDTR) | UP | MG |
| Solyc02g082770 | Solyc02g082770.3.1 | Phosphoinositide phosphatase family protein (AHRD V3.3 *-* AT3G51460.1) | UP | MG |
| Solyc02g091030 | Solyc02g091030.3.1 | Nuclear transcription factor Y subunit C-2 (AHRD V3.3 *-* W9SQ52_9ROSA) | UP | MG |
| Solyc11g008530 | Solyc11g008530.2.1 | Dicer-like 2d | UP | MG |
| Solyc02g088950 | Solyc02g088950.2.1 | Superoxide dismutase [Cu—Zn] (AHRD V3.3 --* A9NTC6_PICSI) | UP | MG |
| Solyc05g013300 | Solyc05g013300.1.1 | Pseudomonas syringae pv tomato resis. | UP | MG |
| Solyc03g078570 | Solyc03g078570.3.1 | Ras-related protein (AHRD V3.3 *** W9QNC0_9ROSA) | UP | MG |
| Solyc04g050760 | Solyc04g050760.3.1 | U11/U12 small nuclear ribonucleoprotein (AHRD V3.3 *** AT3G04160.3) | UP | MG |
| Solyc08g023570 | Solyc08g023570.2.1 | WD repeat-containing protein 48 (AHRD V3.3 *-* A0A1D1XI50_9ARAE) | UP | MG |
| Solyc07g053250 | Solyc07g053250.3.1 | F-box protein family (AHRD V3.3 --* A0A151SU61_CAJCA) | UP | MG |
| Solyc11g030550 | Solyc11g030550.2.1 | Cyclin family protein (AHRD V3.3 *-* AT5G06150.2) | UP | MG |
| Solyc08g061880 | Solyc08g061880.2.1 | Peroxisome biogenesis factor 10 (AHRD V3.3 *-* B9U2H6_TOBAC) | UP | MG |
| Solyc01g104160 | Solyc01g104160.3.1 | arginine/glutamate-rich 1 protein (AHRD V3.3 *** AT1G10890.2) | UP | MG |
| Solyc04g057990 | Solyc04g057990.3.1 | Zinc finger transcription factor 33 | UP | MG |
| Solyc07g052720 | Solyc07g052720.3.1 | MADS-box transcription factor (AHRD V3.3 *** A0A072UYG7_MEDTR) | UP | MG |
| Solyc02g089010 | Solyc02g089010.2.1 | Receptor protein kinase, putative (AHRD V3.3 *-* B9RXG1_RICCO) | UP | MG |
| Solyc09g074730 | Solyc09g074730.2.1 | Homeobox protein LUMINIDEPENDENS (AHRD V3.3 *** | UP | MG |
| | | A0A0B2S9W0_GLYSO) | | |
| Solyc01g067570 | Solyc01g067570.3.1 | Adaptin family protein (AHRD V3.3 --* AT4G23460.1) | UP | MG |
| Solyc01g090860 | Solyc01g090860.3.1 | Nucleotidyltransferase family protein (AHRD V3.3 *** AT4G00060.4) | UP | MG |
| Solyc02g067270 | Solyc02g067270.2.1 | RING/U-box superfamily protein (AHRD V3.3 *-* AT1G26800.1) | UP | MG |
| Solyc10g076270 | Solyc10g076270.2.1 | Katanin p80 WD40 repeat-containing subunit B1 homolog (AHRD V3.3 *-* | UP | MG |
| | | I1HS93_BRADI) | | |
| Solyc12g027540 | Solyc12g027540.2.1 | Retrovirus-related Pol polyprotein from transposon TNT 1-94 (AHRD V3.3 *-* | UP | MG |
| | | A0A151RVI7_CAJCA) | | |
| Solyc07g032170 | Solyc07g032170.3.1 | alpha/beta-Hydrolases superfamily protein (AHRD V3.3 *-* AT4G24160.2) | UP | MG |
| Solyc02g036290 | Solyc02g036290.3.1 | malonyl-CoA decarboxylase family protein (AHRD V3.3 *-* AT4G04320.2) | UP | MG |
| Solyc02g091620 | Solyc02g091620.2.1 | Nuclear pore complex protein (AHRD V3.3 --* AT1G55540.2) | UP | MG |
| Solyc02g084285 | Solyc02g084285.1.1 | Nuclear pore complex protein (AHRD V3.3 --* W9SV07_9ROSA) | UP | MG |
| Solyc05g047540 | Solyc05g047540.3.1 | Pentatricopeptide repeat superfamily protein, putative (AHRD V3.3 *** | UP | MG |
| | | A0A061GYI5_THECC) | | |
| Solyc01g104410 | Solyc01g104410.3.1 | Sterol glucosyltransferase-like protein (AHRD V3.3 *** A0A072V719_MEDTR) | UP | MG |
| Solyc01g007980 | Solyc01g007980.3.1 | Protein kinase family protein (AHRD V3.3 *** C6ZRU6_SOYBN) | UP | MG |
| Solyc01g016460 | Solyc01g016460.3.1 | Phototropic-responsive NPH3 family protein (AHRD V3.3 --* AT1G67900.6) | UP | MG |
| Solyc02g078490 | Solyc02g078490.3.1 | TSA: Wollemia nobilis Ref_Wollemi_Transcript_5456_2539 transcribed RNA | UP | MG |
| | | sequence (AHRD V3.3 --* A0A0C9RPH5_9SPER) | | |
| Solyc12g088920 | Solyc12g088920.2.1 | Calcineurin-like metallo-phosphoesterase superfamily protein (AHRD V3.3 *** | UP | MG |
| | | AT4G11800.1) | | |
| Solyc04g016550 | Solyc04g016550.3.1 | Folylpolyglutamate synthase (AHRD V3.3 *** FPGS2_ARATH) | UP | MG |
| Solyc00g005094 | Solyc00g005094.1.1 | Glucose-6-phosphate 1-dehydrogenase 3, chloroplastic (AHRD V3.3 --* | UP | MG |
| | | G6PD3_ARATH) | | |
| Solyc01g087410 | Solyc01g087410.3.1 | RING/FYVE/PHD zinc finger protein, putative (AHRD V3.3 *-* | UP | MG |
| | | A0A072UJ97_MEDTR) | | |
| Solyc02g005510 | Solyc02g005510.3.1 | Transcriptional coactivator/pterin dehydratase (AHRD V3.3 *** AT1G29810.1) | UP | MG |
| Solyc11g008540 | Solyc11g008540.2.1 | Dicer-like 2b | UP | MG |
| Solyc02g085220 | Solyc02g085220.3.1 | Vacuolar sorting protein 39 (AHRD V3.3 *** AT4G36630.1) | UP | MG |
| Solyc02g067285 | Solyc02g067285.1.1 | Structural maintenance of chromosomes protein (AHRD V3.3 *-* | UP | MG |
| | | A0A068U3R5_COFCA) | | |
| Solyc01g106947 | Solyc01g106947.1.1 | Exocyst complex component SEC3A (AHRD V3.3 --* A0A1D1XZ59_9ARAE) | UP | MG |
| Solyc01g108410 | Solyc01g108410.2.1 | Pentatricopeptide repeat-containing protein (AHRD V3.3 *** | UP | MG |
| | | A0A103XM66_CYNCS) | | |
| Solyc01g103610 | Solyc01g103610.3.1 | Disease resistance protein (AHRD V3.3 *** A0A103XDQ0_CYNCS) | UP | MG |
| Solyc03g059160 | Solyc03g059160.2.1 | Ion channel CASTOR (AHRD V3.3 *-* CASTO_LOTJA) | UP | MG |
| Solyc01g097500 | Solyc01g097500.3.1 | LSTK-1-like kinase | UP | MG |
| Solyc10g006210 | Solyc10g006210.2.1 | Endoribonuclease Dicer (AHRD V3.3 *-* A0A0BOP4A5_GOSAR) | UP | MG |
| Solyc05g025905 | Solyc05g025905.1.1 | Starch synthase, chloroplastic/amyloplastic (AHRD V3.3 *-* K4BBV6_SOLLC) | UP | MG |
| Solyc07g053255 | Solyc07g053255.1.1 | F-box protein (AHRD V3.3 *-* G7KUJ4_MEDTR) | UP | MG |
| Solyc01g058260 | Solyc01g058260.3.1 | Poly(A) polymerase, putative (AHRD V3.3 *** B9RZU0_RICCO) | UP | MG |
| Solyc01g057477 | Solyc01g057477.1.1 | mediator of RNA polymerase II transcription subunit 15a-like protein (AHRD | UP | MG |
| | | V3.3 --* AT1G15780.3) | | |
| Solyc06g066460 | Solyc06g066460.3.1 | Poly(A) polymerase (AHRD V3.3 *-* A0A0B2Q923_GLYSO) | UP | MG |
| Solyc08g060970 | Solyc08g060970.3.1 | polygalacturonase AF118567 | UP | MG |
| Solyc04g072010 | Solyc04g072010.3.1 | carboxypeptidase D (AHRD V3.3 *** AT1G71696.2) | UP | MG |
| Solyc02g089515 | Solyc02g089515.1.1 | NAD(P)H-quinone oxidoreductase subunit K, chloroplastic (AHRD V3.3 --* | UP | MG |
| | | NDHK_NUPAD) | | |
| Solyc09g014415 | Solyc09g014415.1.1 | BTB/POZ domain-containing protein (AHRD V3.3 *** AT3G09030.1) | UP | MG |
| Solyc04g016010 | Solyc04g016010.3.1 | Thioredoxin reductase (AHRD V3.3 *** M1C6NO_SOLTU) | UP | MG |
| Solyc01g106205 | Solyc01g106205.1.1 | myosin-M heavy protein (AHRD V3.3 --* AT3G54060.2) | UP | MG |
| Solyc06g071620 | Solyc06g071620.3.1 | U5 small nuclear ribonucleoprotein helicase (AHRD V3.3 *** AT1G20960.2) | UP | MG |
| Solyc12g077460 | Solyc12g077460.2.1 | WD-40 repeat family protein/beige-like protein (AHRD V3.3 --* AT2G45540.6) | UP | MG |
| Solyc03g058930 | Solyc03g058930.3.1 | Hypersensitive-induced response protein (AHRD V3.3 *** Q6UNT3_CUCSA) | UP | MG |
| Solyc05g016395 | Solyc05g016395.1.1 | Rhomboid family protein (AHRD V3.3 *** B9HXC8_POPTR) | UP | MG |
| Solyc02g082613 | Solyc02g082613.1.1 | Phototropic-responsive NPH3 family protein (AHRD V3.3 --* AT1G03010.3) | UP | MG |
| Solyc02g090500 | Solyc02g090500.3.1 | Phenylalanine ammonia-lyase (AHRD V3.3 --* D3K195_9ROSA) | UP | MG |
| Solyc05g005620 | Solyc05g005620.3.1 | RNA-binding 25 (AHRD V3.3 *** A0A0B0MSI7_GOSAR) | UP | MG |
| Solyc06g066473 | Solyc06g066473.1.1 | Poly(A) polymerase (AHRD V3.3 *-* W9RV28_9ROSA) | UP | MG |
| Solyc12g036800 | Solyc12g036800.1.1 | Receptor like protein (AHRD V3.3 *-* A2V882_TOBAC) | UP | MG |
| Solyc04g014400 | Solyc04g014400.3.1 | LRR receptor-like kinase (AHRD V3.3 *-* A0A072U1S2_MEDTR) | UP | MG |
| Solyc02g089490 | Solyc02g089490.3.1 | Plant Tudor-like RNA-binding protein (AHRD V3.3 --* AT4G32440.3) | UP | MG |
| Solyc06g011550 | Solyc06g011550.3.1 | Myosin-11 (AHRD V3.3 *-* MYO11_ARATH) | UP | MG |
| Solyc02g084315 | Solyc02g084315.1.1 | embryo defective 2410 (AHRD V3.3 --* AT2G25660.4) | UP | MG |
| Solyc05g011970 | Solyc05g011970.3.1 | Cytochrome P450 (AHRD V3.3 *** A0A103XV52_CYNCS) | UP | MG |
| Solyc02g084300 | Solyc02g084300.1.1 | CTP synthase (AHRD V3.3 *-* M1B2B1_SOLTU) | UP | MG |
| Solyc02g021060 | Solyc02g021060.3.1 | Uracil-DNA glycosylase (AHRD V3.3 *** K4B4V6_SOLLC) | UP | MG |
| Solyc01g057340 | Solyc01g057340.3.1 | WD-40 repeat family protein-2 (AHRD V3.3 --* A0A172CCS3_POPTO) | UP | MG |
| Solyc03g120910 | Solyc03g120910.3.1 | Homeobox leucine-zipper protein (AHRD V3.3 *** Q76CL1_ZINVI) | UP | MG |
| Solyc02g088958 | Solyc02g088958.1.1 | telomeric repeat binding protein 1 (AHRD V3.3 --* AT5G59430.5) | UP | MG |
| Solyc11g012550 | Solyc11g012550.2.1 | F-box family protein, putative (AHRD V3.3 *** A0A061GHK4_THECC) | UP | MG |
| Solyc09g055570 | Solyc09g055570.3.1 | Leucine-rich repeat protein kinase family protein (AHRD V3.3 *** AT1G06840.1) | UP | MG |
| Solyc07g007840 | Solyc07g007840.2.1 | SPOC domain/Transcription elongation factor S-II protein (AHRD V3.3 *-* | UP | MG |
| | | AT2G25640.1) | | |
| Solyc03g033825 | Solyc03g033825.1.1 | Ubiquitin-related modifier 1 homolog (AHRD V3.3 --* URM1_ORYSJ) | UP | MG |
| Solyc10g006190 | Solyc10g006190.3.1 | Cell differentiation protein red1, putative (AHRD V3.3 *** B9STZ0_RICCO) | UP | MG |
| Solyc04g005540 | Solyc04g005540.3.1 | Disease resistance protein (NBS-LRR class) family (AHRD V3.3 *-* | UP | MG |
| | | AT5G38350.1) | | |
| Solyc02g082767 | Solyc02g082767.1.1 | ARM repeat superfamily protein (AHRD V3.3 --* AT3G60740.3) | UP | MG |
| Solyc02g067275 | Solyc02g067275.1.1 | Dehydration-responsive protein RD22 (AHRD V3.3 --* K7XKT0_SOLTU) | UP | MG |
| Solyc00g005040 | Solyc00g005040.3.1 | Potassium channel (AHRD V3.3 *-* DOEM91_9ROSI) | UP | MG |
| Solyc02g084310 | Solyc02g084310.2.1 | CTP synthase (AHRD V3.3 *-* M1BXT3_SOLTU) | UP | MG |
| Solyc07g055020 | Solyc07g055020.3.1 | F-box protein family (AHRD V3.3 *** A0A151SU61_CAJCA) | UP | MG |
| Solyc06g060140 | Solyc06g060140.3.1 | Kinesin-like protein (AHRD V3.3 *** A0A061FI31_THECC) | UP | MG |
| Solyc01g081320 | Solyc01g081320.3.1 | Pentatricopeptide repeat-containing protein, putative (AHRD V3.3 *** | UP | MG |
| | | B9RJS7_RICCO) | | |
| Solyc03g115190 | Solyc03g115190.3.1 | Phosphoglycerate mutase family protein (AHRD V3.3 --* AT5G64460.5) | UP | MG |
| Solyc03g111410 | Solyc03g111410.3.1 | B3 domain-containing protein (AHRD V3.3 *-* W9S5V1_9ROSA) | UP | MG |
| Solyc11g030475 | Solyc11g030475.1.1 | Mannosyltransferase (AHRD V3.3 *-* K4AYT1_SOLLC) | UP | MG |
| Solyc01g016455 | Solyc01g016455.1.1 | Ubiquitin carboxyl-terminal hydrolase-related protein (AHRD V3.3 --* | UP | MG |
| | | AT3G47890.2) | | |
| Solyc02g021063 | Solyc02g021063.1.1 | Unknown protein (AHRD V3.3 ) | UP | MG |
| Solyc04g080390 | Solyc04g080390.3.1 | plasma membrane fusion protein (AHRD V3.3 *** AT5G42765.1) | UP | MG |
| Solyc02g092600 | Solyc02g092600.3.1 | Phototropic-responsive NPH3 family protein (AHRD V3.3 *-* | UP | MG |
| | | A0A061DGZ5_THECC) | | |
| Solyc02g082763 | Solyc02g082763.1.1 | Phosphoinositide phosphatase family protein (AHRD V3.3 *-* AT5G66020.1) | UP | MG |
| Solyc07g008475 | Solyc07g008475.1.1 | Subtilisin-like protease (AHRD V3.3 *-* A0A1D1Z5J1_9ARAE) | UP | MG |
| Solyc03g080010 | Solyc03g080010.3.1 | DNA mismatch repair protein MutS2-like (AHRD V3.3 *** Q9LVW1_ARATH) | UP | MG |
| Solyc05g021535 | Solyc05g021535.1.1 | calmodulin-binding transcription activator (AHRD V3.3 --* AT3G16940.3) | UP | MG |
| Solyc02g090505 | Solyc02g090505.1.1 | haloacid dehalogenase-like hydrolase family protein (AHRD V3.3 *-* | UP | MG |
| | | AT1G56500.2) | | |
| Solyc05g021510 | Solyc05g021510.2.1 | Amino acid/polyamine transporter I (AHRD V3.3 --* A0A124SH48_CYNCS) | UP | MG |
| Solyc12g005090 | Solyc12g005090.2.1 | Vacuolar protein sorting-associated protein 35 (AHRD V3.3 *** V4V8H5_9ROSI) | UP | MG |
| Solyc05g014275 | Solyc05g014275.1.1 | HAT dimerisation domain-containing protein-like (AHRD V3.3 *-* | UP | MG |
| | | C8TFH5_ORYSI) | | |
| Solyc01g080310 | Solyc01g080310.3.1 | Pentatricopeptide repeat-containing protein family (AHRD V3.3 *** | UP | MG |
| | | A0A151TET9_CAJCA) | | |
| Solyc06g076253 | Solyc06g076253.1.1 | DCD (Development and cell death) domain protein (AHRD V3.3 *-* | UP | MG |
| | | A0A072V4A1_MEDTR) | | |
| Solyc02g066940 | Solyc02g066940.3.1 | Protein RIK-like protein (AHRD V3.3 *** A0A0B0MTZ1_GOSAR) | UP | MG |
| Solyc02g081620 | Solyc02g081620.3.1 | Zinc finger family protein (AHRD V3.3 *** B9GY13_POPTR) | UP | MG |
| Solyc06g076230 | Solyc06g076230.1.1 | Zinc finger CCCH domain-containing protein 19 (AHRD V3.3 --* C3H19_ORYSJ) | UP | MG |
| Solyc02g088940 | Solyc02g088940.3.1 | CTP synthase family protein (AHRD V3.3 *-* AT1G30820.1) | UP | MG |
| Solyc07g062900 | Solyc07g062900.3.1 | Protein yippee-like (AHRD V3.3 *-* A0A0V0H2B2_SOLCH) | UP | MG |
| Solyc02g088952 | Solyc02g088952.1.1 | DNA mismatch repair protein mutL (AHRD V3.3 --* A0A0B2SBZ1_GLYSO) | UP | MG |
| Solyc01g087290 | Solyc01g087290.3.1 | tRNA-dihydrouridine synthase (AHRD V3.3 *** A0A0V0I8M3_SOLCH) | UP | MG |
| Solyc03g071533 | Solyc03g071533.1.1 | catalase 3 (AHRD V3.3 --* AT1G20620.6) | UP | MG |
| Solyc02g085315 | Solyc02g085315.1.1 | Sec14p-like phosphatidylinositol transfer family protein (AHRD V3.3 --* | UP | MG |
| | | A0A061DH70_THECC) | | |
| Solyc01g006000 | Solyc01g006000.3.1 | GPI mannosyltransferase 2 (AHRD V3.3 *** W9S353_9ROSA) | UP | MG |
| Solyc01g108973 | Solyc01g108973.1.1 | Sorting nexin-16 (AHRD V3.3 *-* W9R163_9ROSA) | UP | MG |
| Solyc11g045110 | Solyc11g045110.2.1 | Sucrose-phosphate synthase family protein (AHRD V3.3 *** AT4G10120.3) | UP | MG |
| Solyc12g036793 | Solyc12g036793.1.1 | Leucine-rich repeat receptor-like protein kinase family protein (AHRD V3.3 *-* | UP | MG |
| | | AT4G08850.1) | | |
| Solyc05g014100 | Solyc05g014100.2.1 | F-box family protein (AHRD V3.3 --* AT3G06240.1) | UP | MG |
| Solyc05g021530 | Solyc05g021530.2.1 | Alcohol dehydrogenase (AHRD V3.3 --* Q4LAW8_CAPCH) | UP | MG |
| Solyc02g089510 | Solyc02g089510.3.1 | UDP-Glycosyltransferase superfamily protein (AHRD V3.3 --* AT3G22250.1) | UP | MG |
| Solyc02g088945 | Solyc02g088945.1.1 | DNA mismatch repair protein MLH3-like protein (AHRD V3.3 --* | UP | MG |
| | | A0A0X7YCF6_SOLTU) | | |
| Solyc05g021555 | Solyc05g021555.1.1 | Alcohol dehydrogenase (AHRD V3.3 *** Q4LAW8_CAPCH) | UP | MG |
| Solyc02g089530 | Solyc02g089530.3.1 | AMP-dependent synthetase and ligase family protein (AHRD V3.3 --* | UP | MG |
| | | AT5G35930.3) | | |
| Solyc06g011540 | Solyc06g011540.3.1 | Myosin (AHRD V3.3 *-* W5ZT55_MAIZE) | UP | MG |
| Solyc01g034150 | Solyc01g034150.3.1 | DNA polymerase eta (AHRD V3.3 *** A0A0B2R9V1_GLYSO) | UP | MG |
| Solyc05g021550 | Solyc05g021550.2.1 | Sister-chromatide cohesion protein 3 (AHRD V3.3 *-* D7M8Q0_ARALL) | UP | MG |
| Solyc03g117370 | Solyc03g117370.3.1 | TOPLESS-related 1 (AHRD V3.3 *-* AT1G80490.3) | UP | MG |
| Solyc06g018004 | Solyc06g018004.1.1 | tetraspanin10 (AHRD V3.3 --* AT1G63260.6) | UP | MG |
| Solyc03g059110 | Solyc03g059110.1.1 | LOW QUALITY: AP2/B3-like transcriptional factor family protein (AHRD | UP | MG |
| | | V3.3 --* AT3G11580.3) | | |
| Solyc04g080285 | Solyc04g080285.1.1 | Pentatricopeptide repeat (PPR) superfamily protein (AHRD V3.3 --* AT3G54980.2) | UP | MG |
| Solyc03g006260 | Solyc03g006260.3.1 | Calcium-binding EF-hand (AHRD V3.3 *-* A0A103XMB0_CYNCS) | DOWN | MG |
| Solyc09g009590 | Solyc09g009590.1.1 | A20/AN1 zinc finger protein | DOWN | MG |
| Solyc02g036350 | Solyc02g036350.3.1 | 1-aminocyclopropane-1-carboxylate oxidase EF501822 | DOWN | MG |
| Solyc02g090120 | Solyc02g090120.1.1 | LOW QUALITY: Inositol 1,4,5-trisphosphate receptor-interacting protein-like 2 | DOWN | MG |
| | | (AHRD V3.3 -** A0A1D1XL30_9ARAE) | | |
| Solyc00g060810 | Solyc00g060810.3.1 | Sn-1 protein (AHRD V3.3 Q42393_CAPAN) | DOWN | MG |
| Solyc06g060840 | Solyc06g060840.1.1 | Oleosin (AHRD V3.3 *** K4C6R4_SOLLC) | DOWN | MG |
| Solyc03g111730 | Solyc03g111730.3.1 | KDEL-tailed cysteine endopeptidase | DOWN | MG |
| Solyc01g088400 | Solyc01g088400.3.1 | ECERIFERUM 1 | DOWN | MG |
| Solyc06g009140 | Solyc06g009140.3.1 | Late embryogenesis abundant protein (AHRD V3.3 *** E3W6T3_SESPO) | DOWN | MG |
| Solyc01g111250 | Solyc01g111250.3.1 | 1-phosphatidylinositol phosphodiesterase-related family protein (AHRD V3.3 *** | DOWN | MG |
| | | B9H168_POPTR) | | |
| Solyc08g082160 | Solyc08g082160.3.1 | Subtilisin-like protease (AHRD V3.3 *-* W9RY25_9ROSA) | DOWN | MG |
| Solyc10g079690 | Solyc10g079690.2.1 | callose synthase 1 (AHRD V3.3 --* AT1G05570.4) | DOWN | MG |
| Solyc12g088220 | Solyc12g088220.2.1 | SIBCAT1 | DOWN | MG |
| Solyc03g120570 | Solyc03g120570.3.1 | Major facilitator superfamily protein (AHRD V3.3 *** AT1G52190.1) | DOWN | MG |
| Solyc06g076320 | Solyc06g076320.1.1 | LOW QUALITY: Rotundifolia-like protein (AHRD V3.3 *** G7JUW7_MEDTR) | DOWN | MG |
| Solyc01g099040 | Solyc01g099040.3.1 | GDSL esterase/lipase (AHRD V3.3 *** A0A199UFA2_ANACO) | DOWN | MG |
| Solyc08g066250 | Solyc08g066250.3.1 | Histidine decarboxylase (AHRD V3.3 *** DCHS_SOLLC) | DOWN | MG |
| Solyc05g013640 | Solyc05g013640.1.1 | DEAD-box ATP-dependent RNA helicase-like protein (AHRD V3.3 --* | DOWN | MG |
| | | AT1G24575.1) | | |
| Solyc05g013620 | Solyc05g013620.2.1 | Retrovirus-related Pol polyprotein from transposon TNT 1-94 (AHRD V3.3 --* | DOWN | MG |
| | | A0A151U3Z8_CAJCA) | | |
| Solyc10g078440 | Solyc10g078440.1.1 | LOW QUALITY: VQ motif family protein (AHRD V3.3 *-* B6TQH0_MAIZE) | DOWN | MG |
| Solyc03g098100 | Solyc03g098100.3.1 | NAD(P)H-dependent oxidoreductase (AHRD V3.3 *** B6TLR8_MAIZE) | DOWN | MG |
| Solyc03g097700 | Solyc03g097700.3.1 | O-methyltransferase (AHRD V3.3 *** F6M2M1_VITPS) | DOWN | MG |
| Solyc03g111720 | Solyc03g111720.3.1 | Peptide methionine sulfoxide reductase (AHRD V3.3 *** MSRA_SOLLC) | DOWN | MG |
| Solyc10g080900 | Solyc10g080900.2.1 | ripening related X72730 | DOWN | MG |
| Solyc12g096570 | Solyc12g096570.1.1 | ARGOS (AHRD V3.3 *** C7SFP7_SOLLC) | DOWN | MG |
| Solyc04g007000 | Solyc04g007000.1.1 | AP2/B3 transcription factor family protein (AHRD V3.3 *** AT1G25560.1) | DOWN | MG |
| Solyc07g006600 | Solyc07g006600.1.1 | glutamine dumper 3 (AHRD V3.3 *** AT5G57685.1) | DOWN | MG |
| Solyc09g005730 | Solyc09g005730.3.1 | Plant protein 1589 of unknown function (AHRD V3.3 *** AT3G55240.1) | DOWN | MG |
| Solyc08g066240 | Solyc08g066240.3.1 | Histidine decarboxylase (AHRD V3.3 *** DCHS_SOLLC) | DOWN | MG |
| Solyc10g052490 | Solyc10g052490.2.1 | Isoflavone reductase homolog (AHRD V3.3 *** IFRH_SOLTU) | DOWN | MG |
| Solyc09g061700 | Solyc09g061700.3.1 | Tetratricopeptide repeat (TPR)-like superfamily protein (AHRD V3.3 *** | UP | Ripe |
| | | AT5G48850.1) | | |
| Solyc09g082860 | Solyc09g082860.3.1 | ATP-sulfurylase (AHRD V3.3 *** Q1W2K0_CAMSI) | UP | Ripe |
| Solyc10g017980 | Solyc10g017980.1.1 | Chitinase (AHRD V3.3 *-* B8QVJ5_ZEAMP) | UP | Ripe |
| Solyc07g062500 | Solyc07g062500.3.1 | Cytochrome P450 (AHRD V3.3 *** A0A124SAX2_CYNCS) | UP | Ripe |
| Solyc09g090900 | Solyc09g090900.3.1 | Aconitate hydratase (AHRD V3.3 *** A0A059LL79_9CHLO) | UP | Ripe |
| Solyc03g034375 | Solyc03g034375.1.1 | Lipid transfer protein (AHRD V3.3 *** S4TID2_GOSHI) | UP | Ripe |
| Solyc07g052250 | Solyc07g052250.3.1 | Unknown protein (AHRD V3.3 ) | UP | Ripe |
| Solyc04g072760 | Solyc04g072760.3.1 | Sulfate transporter (AHRD V3.3 *** S6A1Z9_NICAT) | UP | Ripe |
| Solyc09g098510 | Solyc09g098510.3.1 | Extensin (Class I) (AHRD V3.3 *-* Q01945_SOLLC) | UP | Ripe |
| Solyc08g077330 | Solyc08g077330.3.1 | Expansin-like protein (AHRD V3.3 *** W9SU42_9ROSA) | UP | Ripe |
| Solyc08g006880 | Solyc08g006880.3.1 | Multidrug resistance protein ABC transporter family protein (AHRD V3.3 *** | UP | Ripe |
| | | G7IP56_MEDTR) | | |
| Solyc03g114960 | Solyc03g114960.3.1 | ABC transporter B family protein (AHRD V3.3 *** A0A072URQ8_MEDTR) | UP | Ripe |
| Solyc03g080100 | Solyc03g080100.3.1 | Heavy metal transport/detoxification superfamily protein (AHRD V3.3 *-* | UP | Ripe |
| | | AT5G24580.4) | | |
| Solyc02g092860 | Solyc02g092860.3.1 | Cytochrome P450, putative (AHRD V3.3 *** B9R747_RICCO) | UP | Ripe |
| Solyc07g006145 | Solyc07g006145.1.1 | Cytochrome P450 (AHRD V3.3 *** A9ZT56_COPJA) | UP | Ripe |
| Solyc12g088670 | Solyc12g088670.2.1 | cysteine protease CYP1 | UP | Ripe |
| Solyc02g087770 | Solyc02g087770.3.1 | Aldose 1-epimerase (AHRD V3.3 *** K4BBT3_SOLLC) | UP | Ripe |
| Solyc04g010155 | Solyc04g010155.1.1 | Pax6 (AHRD V3.3 *-* A0A0B0PQB1_GOSAR) | UP | Ripe |
| Solyc11g069940 | Solyc11g069940.1.1 | Glutaredoxin (AHRD V3.3 * A0A118JSL8_CYNCS) | UP | Ripe |
| Solyc02g079500 | Solyc02g079500.4.1 | Peroxidase (AHRD V1 *** Q50LG4_TOBAC) | UP | Ripe |
| Solyc01g099180 | Solyc01g099180.3.1 | Lipoxygenase (AHRD V3.3 *** Q43800_TOBAC) | DOWN | Ripe |
| Solyc07g054790 | Solyc07g054790.1.1 | Wound-responsive family protein (AHRD V3.3 *** A0A061E3U8_THECC) | DOWN | Ripe |
| Solyc07g064180 | Solyc07g064180.3.1 | pectin methylesterase 2 | DOWN | Ripe |
| Solyc09g075420 | Solyc09g075420.3.1 | ethylene response factor E.1 | DOWN | Ripe |
| Solyc06g062600 | Solyc06g062600.3.1 | Glucose-methanol-choline (GMC) oxidoreductase family protein (AHRD V3.3 *** | DOWN | Ripe |
| | | ATIG72970.1) | | |
| Solyc07g026650 | Solyc07g026650.3.1 | 1-aminocyclopropane-1-carboxylate oxidase 5 | DOWN | Ripe |
| Solyc02g085910 | Solyc02g085910.3.1 | LOB domain-containing protein (AHRD V3.3 *** A0A0K9PUI8_ZOSMR) | DOWN | Ripe |
| Solyc07g054770 | Solyc07g054770.1.1 | Wound-responsive family protein (AHRD V3.3 *** A0A061E3U8_THECC) | DOWN | Ripe |
| Solyc06g066430 | Solyc06g066430.3.1 | SUN-like protein 18 | DOWN | Ripe |
| Solyc01g100370 | Solyc01g100370.3.1 | Adenine nucleotide alpha hydrolases-like superfamily protein (AHRD V3.3 *** | DOWN | Ripe |
| | | AT3G62550.1) | | |
| Solyc07g054750 | Solyc07g054750.1.1 | LOW QUALITY: Wound-responsive family protein (AHRD V3.3 *** | DOWN | Ripe |
| | | A0A061E3U8_THECC) | | |
| Solyc10g050980 | Solyc10g050980.1.1 | PAB-dependent poly(A)-specific ribonuclease subunit PAN2 (AHRD V3.3 -** | DOWN | Ripe |
| | | A0A1D1XPR6_9ARAE) | | |
| Solyc06g050870 | Solyc06g050870.3.1 | Hypoxia-responsive family protein (AHRD V3.3 *** A0A061FYY0_THECC) | DOWN | Ripe |
| Solyc04g054340 | Solyc04g054340.1.1 | LOW QUALITY: transmembrane protein (AHRD V3.3 --* AT5G65440.9) | DOWN | Ripe |
| Solyc06g007580 | Solyc06g007580.1.1 | LOW QUALITY: DUF506 family protein (AHRD V3.3 *** G7LF52_MEDTR) | DOWN | Ripe |
| Solyc07g054810 | Solyc07g054810.1.1 | LOW QUALITY: glutamate-1-semialdehyde 2,1-aminomutase 2 (AHRD V3.3 --* | DOWN | Ripe |
| | | AT3G48730.1) | | |
| Solyc12g006560 | Solyc12g006560.2.1 | Early nodulin-93 (AHRD V3.3 *** A0A061GV12_THECC) | DOWN | Ripe |
| Solyc02g090750 | Solyc02g090750.3.1 | Eukaryotic initiation factor 4A (ATP-dependent RNA helicase eIF4A) (AHRD V3.3 | DOWN | Ripe |
| | | *-* E1ZM95_CHLVA) | | |
| Solyc01g109100 | Solyc01g109100.2.1 | LOW QUALITY: phytochrome interacting factor 3-like 5 (AHRD V3.3 --* | DOWN | Ripe |
| | | AT2G20180.6) | | |
| Solyc11g008680 | Solyc11g008680.2.1 | Acyl-[acyl-carrier-protein] desaturase (AHRD V3.3 *** A0A060IKL1_NICBE) | DOWN | Ripe |
| Solyc06g066770 | Solyc06g066770.1.1 | Kelch repeat-containing F-box family protein (AHRD V3.3 *** B9GEJ8_POPTR) | DOWN | Ripe |
| Solyc12g056510 | Solyc12g056510.2.1 | Trihelix transcription factor GT-2 (AHRD V3.3 *** A0A0B2SAG9_GLYSO) | DOWN | Ripe |
| Solyc02g081830 | Solyc02g081830.3.1 | Haloacid dehalogenase-like hydrolase domain-containing protein 3 (AHRD V3.3 | DOWN | Ripe |
| | | *** W9RRW5_9ROSA), Pfam: PF00702 | | |
| Solyc07g065970 | Solyc07g065970.1.1 | Chaperone protein DNAj, putative (AHRD V3.3 *** B9SXA3_RICCO) | DOWN | Ripe |
| Solyc10g050990 | Solyc10g050990.1.1 | Pyruvate kinase (AHRD V3.3 --* A9TZX1_PHYPA) | DOWN | Ripe |

TABLE 4
Gene Ontology Enrichment Analysis of the DEG in adp Fruit

| Cluster | ID | Description | Adjusted p | Gene ID | Count |
|---|---|---|---|---|---|
| Placenta_6dpa_UP | GO: 0034599 | cellular response to oxidative stress | 2.29E−02 | Solyc05g006860/Solyc05g006850 | 2 |
| Placenta_6dpa_UP | GO: 0006571 | tyrosine biosynthetic process | 4.27E−02 | Solyc06g050630 | 1 |
| Placenta_6dpa_UP | GO: 0019346 | transsulfuration | 4.27E−02 | Solyc08g083110 | 1 |
| Placenta_6dpa_UP | GO: 0007205 | protein kinase C-activating G-protein coupled receptor signaling pathway | 4.27E−02 | Solyc03g115370 | 1 |
| Placenta_6dpa_UP | GO: 0009415 | response to water | 4.27E−02 | Solyc01g109920 | 1 |
| Placenta_6dpa_UP | GO: 0009693 | ethylene biosynthetic process | 4.44E−02 | Solyc02g091990 | 1 |
| Placenta_6dpa_UP | GO: 0046486 | glycerolipid metabolic process | 4.54E−02 | Solyc02g094400 | 1 |
| Placenta_6dpa_UP | GO: 0006665 | sphingolipid metabolic process | 4.69E−02 | Solyc10g086690 | 1 |
| Placenta_6dpa_DOWN | GO: 0045490 | pectin catabolic process | 4.98E−02 | Solyc06g009190/Solyc03g058910 | 2 |
| Placenta_6dpa_DOWN | GO: 0006672 | ceramide metabolic process | 4.98E−02 | Solyc08g082920 | 1 |
| Placenta_6dpa_DOWN | GO: 0006880 | intracellular sequestering of iron ion | 4.98E−02 | Solyc01g104780 | 1 |
| Placenta_6dpa_DOWN | GO: 0019252 | starch biosynthetic process | 4.98E−02 | Solyc07g056140 | 1 |
| Placenta_6dpa_DOWN | GO: 0034755 | iron ion transmembrane transport | 4.98E−02 | Solyc01g104780 | 1 |
| Placenta_6dpa_DOWN | GO: 0090156 | cellular sphingolipid homeostasis | 4.98E−02 | Solyc08g082920 | 1 |
| Placenta_20dpa_UP | GO: 0006629 | lipid metabolic process | 1.50E−02 | Solyc07g032180/Solyc06g030590/Solyc07g032220/Solyc06g011530/ | 7 |
| | | | | Solyc03g044910/Solyc07g032170/Solyc09g075790 | |
| Placenta_20dpa_UP | GO: 0006281 | DNA repair | 3.68E−02 | Solyc09g009080/Solyc04g051490/Solyc05g051680/Solyc12g042000/ | 6 |
| | | | | Solyc02g005310/Solyc09g066080 | |
| Placenta_20dpa_UP | GO: 0045944 | positive regulation of transcription by RNA polymerase II | 1.93E−02 | Solyc09g008520/Solyc01g087320/Solyc01g087400/Solyc11g062010/ | 5 |
| | | | | Solyc09g066080 | |
| Placenta_20dpa_UP | GO: 0006457 | protein folding | 3.96E−03 | Solyc11g020330/Solyc03g117630/Solyc02g093600/Solyc09g092690/ | 5 |
| | | | | Solyc03g113930 | |
| Placenta_20dpa_UP | GO: 0009408 | response to heat | 1.86E−02 | Solyc11g020330/Solyc02g072000/Solyc02g093600/Solyc03g113930 | 4 |
| Placenta_20dpa_UP | GO: 0019752 | carboxylic acid metabolic process | 5.57E−03 | Solyc04g049360/Solyc03g112060/Solyc06g011530/Solyc03g044910 | 4 |
| Placenta_20dpa_UP | GO: 0030307 | positive regulation of cell growth | 1.04E−02 | Solyc10g076270/Solyc10g076260/Solyc05g021580/Solyc01g106770 | 4 |
| Placenta_20dpa_UP | GO: 0031929 | TOR signaling | 1.63E−02 | Solyc10g076270/Solyc10g076260/Solyc05g021580/Solyc01g106770 | 4 |
| Placenta_20dpa_UP | GO: 0009651 | response to salt stress | 2.60E−02 | Solyc11g020330/Solyc02g093600/Solyc03g113930 | 3 |
| Placenta_20dpa_UP | GO: 0000302 | response to reactive oxygen species | 1.67E−02 | Solyc11g020330/Solyc02g093600/Solyc03g113930 | 3 |
| Placenta_20dpa_UP | GO: 0006633 | fatty acid biosynthetic process | 4.72E−03 | Solyc03g044910/Solyc02g036290/Solyc09g075790 | 3 |
| Placenta_20dpa_UP | GO: 0009835 | fruit ripening | 3.68E−02 | Solyc07g049530/Solyc10g080210 | 2 |
| Placenta_20dpa_UP | GO: 0031401 | positive regulation of protein modification process | 4.25E−02 | Solyc10g076270/Solyc09g066080 | 2 |
| Placenta_20dpa_UP | GO: 0006665 | sphingolipid metabolic process | 2.50E−02 | Solyc06g030590/Solyc03g044910 | 2 |
| Placenta_20dpa_DOWN | GO: 0015979 | photosynthesis | 4.22E−06 | Solyc02g069450/Solyc06g083680/Solyc08g006930/Solyc03g034220/ | 9 |
| | | | | Solyc10g077040/Solyc03g005760/Solyc03g005780/Solyc01g105050/ | |
| | | | | Solyc03g005770 | |
| Placenta_20dpa_DOWN | GO: 0009416 | response to light stimulus | 1.76E−05 | Solyc03g005760/Solyc03g005780/Solyc03g006880/Solyc01g105050/ | 6 |
| | | | | Solyc03g005770/Solyc02g070430 | |
| Placenta_20dpa_DOWN | GO: 0009765 | photosynthesis, light harvesting | 1.76E−05 | Solyc03g005760/Solyc02g071030/Solyc03g005780/Solyc01g105050/ | 5 |
| | | | | Solyc03g005770 | |
| Placenta_20dpa_DOWN | GO: 0006091 | generation of precursor metabolites and energy | 8.43E−03 | Solyc03g005770/Solyc01g105050/Solyc03g005760/Solyc10g018300/ | 5 |
| | | | | Solyc03g115820 | |
| Placenta_20dpa_DOWN | GO: 0009768 | photosynthesis, light harvesting in photosystem I | 1.47E−04 | Solyc03g005760/Solyc03g005780/Solyc01g105050/Solyc03g005770 | 4 |
| Placenta_20dpa_DOWN | GO: 0018298 | protein-chromophore linkage | 8.59E−04 | Solyc03g005760/Solyc03g005780/Solyc01g105050/Solyc03g005770 | 4 |
| Placenta_20dpa_DOWN | GO: 0006979 | response to oxidative stress | 2.51E−02 | Solyc06g005160/Solyc10g086680/Solyc10g083650 | 3 |
| Placenta_20dpa_DOWN | GO: 0031640 | killing of cells of other organism | 3.18E−02 | Solyc07g007750/Solyc07g007760 | 2 |
| Placenta_20dpa_DOWN | GO: 0006006 | glucose metabolic process | 3.94E−02 | Solyc04g009030/Solyc04g082630 | 2 |
| Placenta_20dpa_DOWN | GO: 0006098 | pentose-phosphate shunt | 2.15E−02 | Solyc10g018300/Solyc03g115820 | 2 |
| Placenta_MG_UP | GO: 0006281 | DNA repair | 1.61E−05 | Solyc01g109080/Solyc02g083900/Solyc02g091120/Solyc11g067140/ | 23 |
| | | | | Solyc11g018510/Solyc01g034150/Solyc09g066080/Solyc09g011930/ | |
| | | | | Solyc03g111790/Solyc09g018690/Solyc12g041980/Solyc03g025890/ | |
| | | | | Solyc09g009080/Solyc01g106770/Solyc05g051680/Solyc12g042000/ | |
| | | | | Solyc01g111870/Solyc03g112950/Solyc01g107510/Solyc02g094590/ | |
| | | | | Solyc02g005310/Solyc01g099220/Solyc02g021060 | |
| Placenta_MG_UP | GO: 0006396 | RNA processing | 5.18E−07 | Solyc06g048960/Solyc11g008530/Solyc10g080130/Solyc02g081960/ | 20 |
| | | | | Solyc10g006180/Solyc11g008540/Solyc01g103690/Solyc04g005690/ | |
| | | | | Solyc09g064590/Solyc01g086890/Solyc10g006210/Solyc11g018770/ | |
| | | | | Solyc10g006200/Solyc10g005130/Solyc03g053105/Solyc01g086900/ | |
| | | | | Solyc10g006153/Solyc08g077850/Solyc04g007510/Solyc01g006710 | |
| Placenta_MG_UP | GO: 0034470 | ncRNA processing | 3.65E−04 | Solyc04g072265/Solyc11g018810/Solyc04g072260/Solyc12g017860/ | 15 |
| | | | | Solyc08g062560/Solyc01g086900/Solyc05g051230/Solyc04g049226/ | |
| | | | | Solyc06g049090/Solyc08g077850/Solyc11g018770/Solyc10g080130/ | |
| | | | | Solyc03g093480/Solyc03g097550/Solyc12g015710 | |
| Placenta_MG_UP | GO: 0006629 | lipid metabolic process | 3.65E−03 | Solyc07g032180/Solyc05g008790/Solyc02g082763/Solyc11g010550/ | 13 |
| | | | | Solyc09g072710/Solyc04g007650/Solyc07g045420/Solyc01g104310/ | |
| | | | | Solyc07g032220/Solyc01g006000/Solyc01g095930/Solyc06g011530/ | |
| | | | | Solyc10g005840 | |
| Placenta_MG_UP | GO: 0090502 | RNA phosphodiester bond hydrolysis, endonucleolytic | 3.64E−02 | Solyc06g048960/Solyc11g008530/Solyc10g006180/Solyc05g013210/ | 9 |
| | | | | Solyc11g008540/Solyc10g006210/Solyc10g006200/Solyc06g082890/ | |
| | | | | Solyc10g005130 | |
| Placenta_MG_UP | GO: 0006974 | cellular response to DNA damage stimulus | 1.26E−03 | Solyc02g091120/Solyc09g066080/Solyc12g041980/Solyc03g025890/ | 8 |
| | | | | Solyc12g042000/Solyc03g112950/Solyc01g107510/Solyc02g021060 | |
| Placenta_MG_UP | GO: 0009793 | embryo development ending in seed dormancy | 4.22E−02 | Solyc01g108910/Solyc06g048460/Solyc02g086500/Solyc03g059420/ | 8 |
| | | | | Solyc01g090200/Solyc01g106950/Solyc01g106770/Solyc09g092100 | |
| Placenta_MG_UP | GO: 0016567 | protein ubiquitination | 1.11E−04 | Solyc11g005650/Solyc11g005640/Solyc07g065630/Solyc10g084760/ | 8 |
| | | | | Solyc06g053210/Solyc04g076620/Solyc01g067273/Solyc01g067210 | |
| Placenta_MG_UP | GO: 0006260 | DNA replication | 1.35E−03 | Solyc01g099460/Solyc07g049720/Solyc03g115530/Solyc11g067140/ | 8 |
| | | | | Solyc12g098860/Solyc01g068260/Solyc11g020770/Solyc01g099450 | |
| Placenta_MG_UP | GO: 0043631 | RNA polyadenylation | 5.18E−07 | Solyc12g095890/Solyc01g058260/Solyc06g066470/Solyc08g066200/ | 7 |
| | | | | Solyc06g066477/Solyc06g066460/Solyc06g066473 | |
| Placenta_MG_UP | GO: 0006075 | (1->3)-beta-D-glucan biosynthetic process | 1.25E−06 | Solyc01g073750/Solyc01g006370/Solyc01g006350/Solyc03g111575/ | 7 |
| | | | | Solyc02g078230/Solyc07g056260/Solyc01g073755 | |
| Placenta_MG_UP | GO: 0090501 | RNA phosphodiester bond hydrolysis | 2.75E−02 | Solyc06g048960/Solyc11g008530/Solyc10g006180/Solyc11g008540/ | 7 |
| | | | | Solyc10g006210/Solyc10g006200/Solyc10g005130 | |
| Placenta_MG_UP | GO: 0046486 | glycerolipid metabolic process | 4.59E−03 | Solyc07g032180/Solyc02g082763/Solyc11g010550/Solyc04g007650/ | 7 |
| | | | | Solyc01g104310/Solyc01g006000/Solyc01g095930 | |
| Placenta_MG_UP | GO: 0030422 | production of siRNA involved in RNA interference | 5.94E−03 | Solyc06g048960/Solyc11g008530/Solyc11g008540/Solyc10g006210/ | 6 |
| | | | | Solyc10g005130/Solyc01g096390 | |
| Placenta_MG_UP | GO: 0000724 | double-strand break repair via homologous recombination | 2.75E−02 | Solyc08g068010/Solyc01g099460/Solyc09g066080/Solyc09g018690/ | 6 |
| | | | | Solyc12g041980/Solyc05g051680 | |
| Placenta_MG_UP | GO: 0000723 | telomere maintenance | 2.65E−03 | Solyc12g062480/Solyc10g006780/Solyc07g021120/Solyc01g111870/ | 6 |
| | | | | Solyc02g091120/Solyc03g112950 | |
| Placenta_MG_UP | GO: 0006298 | mismatch repair | 9.02E−03 | Solyc09g090870/Solyc03g080010/Solyc03g025890/Solyc02g082660/ | 5 |
| | | | | Solyc10g018530 | |
| Placenta_MG_UP | GO: 0006623 | protein targeting to vacuole | 1.62E−02 | Solyc02g090130/Solyc12g099010/Solyc04g012170/Solyc08g077500/ | 5 |
| | | | | Solyc09g009370 | |
| Placenta_MG_UP | GO: 0031929 | TOR signaling | 9.53E−03 | Solyc10g076270/Solyc10g076260/Solyc01g107415/Solyc01g106770/ | 5 |
| | | | | Solyc05g021580 | |
| Placenta_MG_UP | GO: 0051276 | chromosome organization | 3.40E−03 | Solyc05g054200/Solyc06g048460/Solyc12g041980/Solyc09g066080/ | 5 |
| | | | | Solyc08g067190 | |
| Placenta_MG_UP | GO: 0045053 | protein retention in Golgi apparatus | 3.86E−03 | Solyc08g077500/Solyc11g045185/Solyc02g090130/Solyc11g045160/ | 5 |
| | | | | Solyc09g009370 | |
| Placenta_MG_UP | GO: 0031123 | RNA 3′-end processing | 2.48E−03 | Solyc12g095890/Solyc01g058260/Solyc06g066470/Solyc08g066200 | 4 |
| Placenta_MG_UP | GO: 0006378 | mRNA polyadenylation | 2.75E−02 | Solyc12g095890/Solyc01g058260/Solyc06g066470/Solyc08g066200 | 4 |
| Placenta_MG_UP | GO: 0006221 | pyrimidine nucleotide biosynthetic process | 2.75E−02 | Solyc01g098530/Solyc01g098527/Solyc02g084300/Solyc02g088940 | 4 |
| Placenta_MG_UP | GO: 0017148 | negative regulation of translation | 3.05E−02 | Solyc10g006170/Solyc06g075590/Solyc10g006190/Solyc01g090780 | 4 |
| Placenta_MG_UP | GO: 0042147 | retrograde transport, endosome to Golgi | 3.05E−02 | Solyc02g069650/Solyc12g005090/Solyc02g069660/Solyc03g078570 | 4 |
| Placenta_MG_UP | GO: 0006402 | mRNA catabolic process | 4.93E−02 | Solyc10g006157/Solyc10g006170/Solyc10g006190/Solyc10g006153 | 4 |
| Placenta_MG_UP | GO: 0046488 | phosphatidylinositol metabolic process | 4.93E−02 | Solyc12g049590/Solyc04g007650/Solyc04g017595/Solyc04g017620 | 4 |
| Placenta_MG_UP | GO: 0030488 | tRNA methylation | 3.21E−03 | Solyc01g086900/Solyc06g049090/Solyc08g077850/Solyc01g068070 | 4 |
| Placenta_MG_UP | GO: 0000710 | meiotic mismatch repair | 2.75E−02 | Solyc09g090870/Solyc03g025890/Solyc10g018530 | 3 |
| Placenta_MG_UP | GO: 0002943 | tRNA dihydrouridine synthesis | 2.75E−02 | Solyc01g087380/Solyc01g087320/Solyc01g087290 | 3 |
| Placenta_MG_UP | GO: 0009396 | folic acid-containing compound biosynthetic process | 4.22E−02 | Solyc04g016550/Solyc04g049360/Solyc08g048240 | 3 |
| Placenta_MG_UP | GO: 0042127 | regulation of cell proliferation | 4.93E−02 | Solyc05g054200/Solyc09g066080/Solyc12g041980 | 3 |
| Placenta_MG_UP | GO: 0035066 | positive regulation of histone acetylation | 1.72E−03 | Solyc05g054200/Solyc12g041980/Solyc09g066080 | 3 |
| Placenta_MG_UP | GO: 0034058 | endosomal vesicle fusion | 1.19E−03 | Solyc02g085220/Solyc05g044610/Solyc05g044615 | 3 |
| Placenta_MG_DOWN | GO: 0006979 | response to oxidative stress | 3.30E−02 | Solyc10g078890/Solyc01g080410/Solyc02g092580/Solyc02g083480/ | 8 |
| | | | | Solyc04g071890/Solyc01g079820/Solyc03g111720/Solyc04g064690 | |
| Placenta_MG_DOWN | GO: 0030001 | metal ion transport | 3.30E−02 | Solyc03g043640/Solyc03g025800/Solyc05g008300/Solyc03g025795/ | 6 |
| | | | | Solyc02g032100/Solyc03g007870 | |
| Placenta_MG_DOWN | GO: 0051603 | proteolysis involved in cellular protein catabolic process | 4.56E−02 | Solyc01g087970/Solyc02g069110/Solyc05g041540/Solyc03g111730/ | 6 |
| | | | | Solyc01g108490/Solyc05g013920 | |
| Placenta_MG_DOWN | GO: 0009693 | ethylene biosynthetic process | 5.95E−06 | Solyc02g036350/Solyc07g049530/Solyc12g005940/Solyc09g089580/ | 5 |
| | | | | Solyc07g049550 | |
| Placenta_MG_DOWN | GO: 0009607 | response to biotic stimulus | 3.55E−02 | Solyc02g082920/Solyc00g060810/Solyc00g174340/Solyc01g097240/ | 5 |
| | | | | Solyc08g080640 | |
| Placenta_MG_DOWN | GO: 0006032 | chitin catabolic process | 2.08E−02 | Solyc10g055810/Solyc02g082920/Solyc05g050130/Solyc10g055800 | 4 |
| Placenta_MG_DOWN | GO: 0050832 | defense response to fungus | 4.56E−02 | Solyc07g007750/Solyc00g174340/Solyc01g097240/Solyc08g080640 | 4 |
| Placenta_MG_DOWN | GO: 0006720 | isoprenoid metabolic process | 4.20E−02 | Solyc07g056670/Solyc03g032020/Solyc04g083160/Solyc11g072310 | 4 |
| Placenta_MG_DOWN | GO: 0009835 | fruit ripening | 8.07E−03 | Solyc07g049530/Solyc09g089580/Solyc07g049550 | 3 |
| Placenta_MG_DOWN | GO: 0031640 | killing of cells of other organism | 2.08E−02 | Solyc07g007750/Solyc00g174340/Solyc08g080640 | 3 |
| Placenta_MG_DOWN | GO: 0016998 | cell wall macromolecule catabolic process | 3.30E−02 | Solyc10g055810/Solyc02g082920/Solyc10g055800 | 3 |
| Placenta_MG_DOWN | GO: 0009685 | gibberellin metabolic process | 3.90E−02 | Solyc07g056670/Solyc04g083160/Solyc11g072310 | 3 |
| Placenta_RR_UP | GO: 0006629 | lipid metabolic process | 9.06E−03 | Solyc01g109140/Solyc08g080170/Solyc12g100270/Solyc01g103650/ | 15 |
| | | | | Solyc09g061840/Solyc02g038740/Solyc03g019670/Solyc02g084930/ | |
| | | | | Solyc03g122340/Solyc07g007870/Solyc02g084740/Solyc07g045350/ | |
| | | | | Solyc08g080130/Solyc04g079820/Solyc08g029000 | |
| Placenta_RR_UP | GO: 0044550 | secondary metabolite biosynthetic process | 1.56E−03 | Solyc02g065190/Solyc02g090350/Solyc04g078290/Solyc12g045020/ | 11 |
| | | | | Solyc07g052370/Solyc11g030730/Solyc12g042480/Solyc02g092860/ | |
| | | | | Solyc08g079420/Solyc03g112030/Solyc04g083140 | |
| Placenta_RR_UP | GO: 0007049 | cell cycle | 5.59E−04 | Solyc06g073610/Solyc04g082430/Solyc04g071650/Solyc08g066050/ | 11 |
| | | | | Solyc05g006050/Solyc10g080950/Solyc03g032190/Solyc02g087880/ | |
| | | | | Solyc06g076640/Solyc06g065680/Solyc04g082840 | |
| Placenta_RR_UP | GO: 0007018 | microtubule-based movement | 1.19E−05 | Solyc07g064030/Solyc03g119220/Solyc06g075580/Solyc09g010060/ | 10 |
| | | | | Solyc01g098985/Solyc07g065210/Solyc02g084390/Solyc12g098630/ | |
| | | | | Solyc09g010810/Solyc11g071730 | |
| Placenta_RR_UP | GO: 0009664 | plant-type cell wall organization | 2.38E−02 | Solyc12g098757/Solyc09g098510/Solyc04g071080/Solyc12g098780/ | 7 |
| | | | | Solyc05g007830/Solyc02g088100/Solyc04g071085 | |
| Placenta_RR_UP | GO: 0006559 | L-phenylalanine catabolic process | 2.93E−06 | Solyc09g007900/Solyc09g007920/Solyc10g011920/Solyc00g282510/ | 6 |
| | | | | Solyc09g007890/Solyc09g007910 | |
| Placenta_RR_UP | GO: 0016125 | sterol metabolic process | 4.15E−02 | Solyc01g109140/Solyc12g100270/Solyc02g038740/Solyc02g084930/ | 5 |
| | | | | Solyc02g084740 | |
| Placenta_RR_UP | GO: 0009800 | cinnamic acid biosynthetic process | 6.54E−05 | Solyc09g007900/Solyc09g007920/Solyc09g007890/Solyc09g007910 | 4 |
| Placenta_RR_UP | GO: 0009698 | phenylpropanoid metabolic process | 2.23E−04 | Solyc09g007900/Solyc09g007920/Solyc09g007890/Solyc09g007910 | 4 |
| Placenta_RR_UP | GO: 1902358 | sulfate transmembrane transport | 2.65E−03 | Solyc05g054740/Solyc04g072760/Solyc12g056930/Solyc04g054730 | 4 |
| Placenta_RR_UP | GO: 0008272 | sulfate transport | 3.86E−03 | Solyc05g054740/Solyc04g072760/Solyc12g056930/Solyc04g054730 | 4 |
| Placenta_RR_UP | GO: 0009558 | embryo sac cellularization | 4.97E−02 | Solyc03g096890/Solyc03g119220 | 2 |
| Placenta_RR_UP | GO: 0048443 | stamen development | 4.97E−02 | Solyc02g084740/Solyc09g008240 | 2 |
| Placenta_RR_DOWN | GO: 0009835 | fruit ripening | 1.32E−02 | Solyc07g064180/Solyc07g064170 | 2 |
| Placenta_RR_DOWN | GO: 0031408 | oxylipin biosynthetic process | 4.54E−02 | Solyc01g099180/Solyc01g099190 | 2 |
| Placenta_RR_DOWN | GO: 1901605 | alpha-amino acid metabolic process | 2.88E−02 | Solyc08g008000/Solyc03g083440 | 2 |
| Pericarp_6dpa_UP | GO: 0031408 | oxylipin biosynthetic process | 2.75E−02 | Solyc01g099180/Solyc01g099190 | 2 |
| Pericarp_6dpa_UP | GO: 0000973 | posttranscriptional tethering of RNA polymerase II gene DNA at | 3.55E−02 | Solyc01g104434/Solyc02g084285 | 2 |
| | | nuclear periphery | | | |
| Pericarp_6dpa_UP | GO: 0015996 | chlorophyll catabolic process | 4.11E−02 | Solyc12g005300 | 1 |
| Pericarp_6dpa_DOWN | GO: 0045490 | pectin catabolic process | 3.02E−03 | Solyc02g080220/Solyc02g080200/Solyc03g058910 | 3 |
| Pericarp_6dpa_DOWN | GO: 0042545 | cell wall modification | 2.93E−02 | Solyc02g080220/Solyc02g080200 | 2 |
| Pericarp_6dpa_DOWN | GO: 0071555 | cell wall organization | 2.93E−02 | Solyc02g080220/Solyc02g080200 | 2 |
| Pericarp_6dpa_DOWN | GO: 0009617 | response to bacterium | 2.93E−02 | Solyc04g081560 | 1 |
| Pericarp_6dpa_DOWN | GO: 0009070 | serine family amino acid biosynthetic process | 2.93E−02 | Solyc11g020060 | 1 |
| Pericarp_6dpa_DOWN | GO: 0006379 | mRNA cleavage | 2.93E−02 | Solyc05g055890 | 1 |
| Pericarp_6dpa_DOWN | GO: 0019252 | starch biosynthetic process | 2.93E−02 | Solyc07g056140 | 1 |
| Pericarp_6dpa_DOWN | GO: 0000911 | cytokinesis by cell plate formation | 3.07E−02 | Solyc01g108670 | 1 |
| Pericarp_6dpa_DOWN | GO: 0009723 | response to ethylene | 3.18E−02 | Solyc04g081560 | 1 |
| Pericarp_6dpa_DOWN | GO: 0010215 | cellulose microfibril organization | 4.51E−02 | Solyc03g114890 | 1 |
| Pericarp_6dpa_DOWN | GO: 0045892 | negative regulation of transcription, DNA-templated | 4.51E−02 | Solyc08g068170 | 1 |
| Pericarp_6dpa_DOWN | GO: 0006032 | chitin catabolic process | 4.92E−02 | Solyc07g005100 | 1 |
| Pericarp_20dpa_UP | GO: 0051259 | protein complex oligomerization | 2.44E−02 | Solyc11g020330/Solyc02g093600/Solyc03g113930 | 3 |
| Pericarp_20dpa_UP | GO: 0009651 | response to salt stress | 2.99E−02 | Solyc11g020330/Solyc02g093600/Solyc03g113930 | 3 |
| Pericarp_20dpa_UP | GO: 0042542 | response to hydrogen peroxide | 2.14E−02 | Solyc11g020330/Solyc02g093600/Solyc03g113930 | 3 |
| Pericarp_20dpa_UP | GO: 0009408 | response to heat | 3.01E−02 | Solyc11g020330/Solyc02g093600/Solyc03g113930 | 3 |
| Pericarp_20dpa_DOWN | GO: 0043086 | negative regulation of catalytic activity | 2.27E−03 | Solyc09g090990/Solyc03g123620/Solyc06g009190 | 3 |
| Pericarp_20dpa_DOWN | GO: 0042545 | cell wall modification | 1.67E−02 | Solyc03g123620/Solyc06g009190 | 2 |
| Pericarp_20dpa_DOWN | GO: 0045490 | pectin catabolic process | 1.67E−02 | Solyc03g123620/Solyc06g009190 | 2 |
| Pericarp_20dpa_DOWN | GO: 0009058 | biosynthetic process | 2.59E−02 | Solyc07g056140/Solyc02g082900 | 2 |
| Pericarp_20dpa_DOWN | GO: 0009693 | ethylene biosynthetic process | 2.89E−02 | Solyc02g036350 | 1 |
| Pericarp_20dpa_DOWN | GO: 0019252 | starch biosynthetic process | 2.89E−02 | Solyc07g056140 | 1 |
| Pericarp_20dpa_DOWN | GO: 0031408 | oxylipin biosynthetic process | 4.83E−02 | Solyc03g122340 | 1 |
| Pericarp_MG_UP | GO: 0006952 | defense response | 1.85E−05 | Solyc04g077270/Solyc03g120110/Solyc02g080040/Solyc01g113620/ | 24 |
| | | | | Solyc07g009230/Solyc09g014530/Solyc02g082920/Solyc07g063820/ | |
| | | | | Solyc05g007170/Solyc02g080070/Solyc10g006710/Solyc04g005540/ | |
| | | | | Solyc00g174340/Solyc02g077370/Solyc01g103610/Solyc02g079580/ | |
| | | | | Solyc01g007980/Solyc01g097240/Solyc10g006720/Solyc02g082930/ | |
| | | | | Solyc02g080080/Solyc02g080010/Solyc07g007760/Solyc06g050810 | |
| Pericarp_MG_UP | GO: 0006629 | lipid metabolic process | 1.09E−02 | Solyc07g032180/Solyc06g048570/Solyc02g082763/Solyc01g103650/ | 12 |
| | | | | Solyc02g086180/Solyc01g080900/Solyc06g035960/Solyc00g009110/ | |
| | | | | Solyc04g072020/Solyc01g006000/Solyc06g011530/Solyc03g044790 | |
| Pericarp_MG_UP | GO: 0007049 | cell cycle | 4.61E−02 | Solyc12g008665/Solyc03g117530/Solyc08g066050/Solyc10g080950/ | 10 |
| | | | | Solyc11g030550/Solyc09g075550/Solyc02g087880/Solyc01g087500/ | |
| | | | | Solyc08g007330/Solyc04g082840 | |
| Pericarp_MG_UP | GO: 0006281 | DNA repair | 3.02E−02 | Solyc08g074740/Solyc03g117530/Solyc02g021060/Solyc01g034150/ | 8 |
| | | | | Solyc05g006120/Solyc01g105520/Solyc08g007330/Solyc09g066080 | |
| Pericarp_MG_UP | GO: 0009407 | toxin catabolic process | 1.76E−02 | Solyc09g011600/Solyc07g056470/Solyc09g011590/Solyc09g011630/ | 6 |
| | | | | Solyc01g086680/Solyc09g011550 | |
| Pericarp_MG_UP | GO: 0051276 | chromosome organization | 3.99E−02 | Solyc03g117530/Solyc02g084285/Solyc02g091020/Solyc01g087500/ | 5 |
| | | | | Solyc03g120200 | |
| Pericarp_MG_UP | GO: 0016998 | cell wall macromolecule catabolic process | 2.87E−02 | Solyc02g082920/Solyc10g055800/Solyc11g066270/Solyc02g082930 | 4 |
| Pericarp_MG_UP | GO: 0043631 | RNA polyadenylation | 2.90E−02 | Solyc01g058260/Solyc06g066460/Solyc06g066473 | 3 |
| Pericarp_MG_DOWN | GO: 0071555 | cell wall organization | 5.26E−06 | Solyc03g093110/Solyc07g064180/Solyc07g064190/Solyc03g093120/ | 10 |
| | | | | Solyc03g093130/Solyc03g093080/Solyc07g064170/Solyc07g056000/ | |
| | | | | Solyc10g080210/Solyc07g009380 | |
| Pericarp_MG_DOWN | GO: 0006629 | lipid metabolic process | 1.84E−02 | Solyc01g099190/Solyc04g078900/Solyc01g006540/Solyc03g031860/ | 9 |
| | | | | Solyc08g005610/Solyc02g070430/Solyc04g079730/Solyc01g088400/ | |
| | | | | Solyc01g099180 | |
| Pericarp_MG_DOWN | GO: 0006073 | cellular glucan metabolic process | 6.29E−06 | Solyc03g093110/Solyc03g093120/Solyc03g093130/Solyc03g093080/ | 6 |
| | | | | Solyc07g056000/Solyc07g009380 | |
| Pericarp_MG_DOWN | GO: 0010411 | xyloglucan metabolic process | 6.29E−06 | Solyc03g093110/Solyc03g093120/Solyc03g093130/Solyc03g093080/ | 6 |
| | | | | Solyc07g056000/Solyc07g009380 | |
| Pericarp_MG_DOWN | GO: 0042546 | cell wall biogenesis | 7.13E−06 | Solyc03g093110/Solyc03g093120/Solyc03g093130/Solyc03g093080/ | 6 |
| | | | | Solyc07g056000/Solyc07g009380 | |
| Pericarp_MG_DOWN | GO: 0006950 | response to stress | 5.03E−04 | Solyc07g064160/Solyc04g014600/Solyc06g009050/Solyc06g009140/ | 6 |
| | | | | Solyc02g084840/Solyc02g062390 | |
| Pericarp_MG_DOWN | GO: 0006633 | fatty acid biosynthetic process | 1.44E−03 | Solyc01g099180/Solyc01g006540/Solyc01g099190/Solyc04g079730/ | 6 |
| | | | | Solyc01g088400/Solyc02g085870 | |
| Pericarp_MG_DOWN | GO: 0009058 | biosynthetic process | 4.57E−03 | Solyc08g081550/Solyc07g056140/Solyc10g007110/Solyc01g095080/ | 6 |
| | | | | Solyc03g031860/Solyc05g050010 | |
| Pericarp_MG_DOWN | GO: 0045490 | pectin catabolic process | 5.40E−03 | Solyc07g064180/Solyc03g123620/Solyc07g064190/Solyc07g064170/ | 5 |
| | | | | Solyc05g055510 | |
| Pericarp_MG_DOWN | GO: 0043086 | negative regulation of catalytic activity | 1.47E−02 | Solyc07g064180/Solyc03g123620/Solyc07g064190/Solyc07g064170/ | 5 |
| | | | | Solyc01g088590 | |
| Pericarp_MG_DOWN | GO: 0009835 | fruit ripening | 6.69E−06 | Solyc07g064180/Solyc07g064170/Solyc01g095080/Solyc10g080210 | 4 |
| Pericarp_MG_DOWN | GO: 0031408 | oxylipin biosynthetic process | 2.91E−04 | Solyc01g099180/Solyc01g006540/Solyc01g099190/Solyc04g079730 | 4 |
| Pericarp_MG_DOWN | GO: 0006869 | lipid transport | 9.71E−03 | Solyc01g090350/Solyc02g086310/Solyc01g090360/Solyc05g015490 | 4 |
| Pericarp_MG_DOWN | GO: 0042545 | cell wall modification | 1.84E−02 | Solyc07g064180/Solyc03g123620/Solyc07g064190/Solyc07g064170 | 4 |
| Pericarp_MG_DOWN | GO: 0009414 | response to water deprivation | 3.61E−02 | Solyc08g082210/Solyc02g084840/Solyc02g062390 | 3 |
| Pericarp_MG_DOWN | GO: 0009415 | response to water | 1.08E−02 | Solyc02g084840/Solyc02g062390 | 2 |
| Pericarp_MG_DOWN | GO: 0009693 | ethylene biosynthetic process | 1.47E−02 | Solyc02g036350/Solyc01g095080 | 2 |
| Pericarp_MG_DOWN | GO: 0019953 | sexual reproduction | 1.84E−02 | Solyc06g034040/Solyc06g060840 | 2 |
| Pericarp_MG_DOWN | GO: 0009631 | cold acclimation | 2.48E−02 | Solyc02g084840/Solyc02g062390 | 2 |
| Pericarp_MG_DOWN | GO: 0019915 | lipid storage | 2.48E−02 | Solyc06g034040/Solyc06g060840 | 2 |
| Pericarp_RR_UP | GO: 0044281 | small molecule metabolic process | 1.59E−03 | Solyc11g007990/Solyc12g056830/Solyc04g082630/Solyc02g065280/ | 13 |
| | | | | Solyc04g009960/Solyc02g086900/Solyc02g080540/Solyc02g089620/ | |
| | | | | Solyc12g008430/Solyc12g006450/Solyc07g021750/Solyc04g009030/ | |
| | | | | Solyc10g086730 | |
| Pericarp_RR_UP | GO: 0006082 | organic acid metabolic process | 2.41E−02 | Solyc11g007990/Solyc02g065280/Solyc04g009960/Solyc02g089620/ | 6 |
| | | | | Solyc12g008430/Solyc12g006450 | |
| Pericarp_RR_UP | GO: 0006091 | generation of precursor metabolites and energy | 4.00E−02 | Solyc11g007990/Solyc12g056830/Solyc07g047850/Solyc03g115900/ | 5 |
| | | | | Solyc08g075540 | |
| Pericarp_RR_UP | GO: 0019318 | hexose metabolic process | 4.43E−02 | Solyc04g082630/Solyc04g009030/Solyc10g086730 | 3 |
| Pericarp_RR_UP | GO: 0006108 | malate metabolic process | 4.98E−02 | Solyc11g007990/Solyc12g008430 | 2 |
| Pericarp_RR_DOWN | GO: 0006633 | fatty acid biosynthetic process | 1.31E−03 | Solyc01g099180/Solyc06g053480/Solyc11g008680/Solyc01g099200/ | 5 |
| | | | | Solyc08g014000 | |
| Pericarp_RR_DOWN | GO: 0006096 | glycolytic process | 1.31E−03 | Solyc08g066100/Solyc04g011510/Solyc04g082880/Solyc09g009260 | 4 |
| Pericarp_RR_DOWN | GO: 0031408 | oxylipin biosynthetic process | 1.31E−03 | Solyc01g099180/Solyc01g099200/Solyc08g014000 | 3 |
| Pericarp_RR_DOWN | GO: 0006631 | fatty acid metabolic process | 3.85E−03 | Solyc06g053480/Solyc11g008680/Solyc08g014000 | 3 |
| Pericarp_RR_DOWN | GO: 0061615 | glycolytic process through fructose-6-phosphate | 1.08E−02 | Solyc08g066100/Solyc04g082880 | 2 |
| Pericarp_RR_DOWN | GO: 0006950 | response to stress | 3.59E−02 | Solyc07g064160/Solyc01g100370/Solyc09g075210 | 3 |
| Pericarp_RR_DOWN | GO: 0015743 | malate transport | 4.59E−02 | Solyc11g010500 | 1 |

TABLE 5
Examples of DEGs in adp Placenta During Fruit Growth

| Locus | Identifier (ITAG 3.2) | Annotation (ITAG 3.2) | FC adp/CL | FC adp/WT | UP/DOWN | Stage | Biological pathway |
|---|---|---|---|---|---|---|---|
| Solyc05g013490 | Solyc05g013490.3.1 | Cyclin-related family protein (AHRD V3.3 *** | 2.31527719 | 1.87042735 | UP | 20 DPA | Cell cycle |
| | | B9HLR1_POPTR) | | | | | |
| Solyc11g030550 | Solyc11g030550.2.1 | Cyclin B2 | 2.023072061 | 1.595264798 | UP | 20 DPA | Cell cycle |
| Solyc09g065610 | Solyc09g065610.2.1 | Bax inhibitor (AHRD V3.3 *—* | 1.526055015 | 2.297522078 | UP | 6 DPA | Cell death regulation |
| | | A0A0K1ETI6_TECGR) | | | | | |
| Solyc01g091590 | Solyc01g091590.3.1 | LOW QUALITY: BON1-associated protein 2 | 1.658871422 | 1.516309081 | UP | 6 DPA | Cell death regulation |
| | | (AHRD V3.3 *** W9QRQ0_9ROSA) | | | | | |
| Solyc04g076710 | Solyc04g076710.3.1 | COBRA-like protein 11 precursor (AHRD V3.3 ——* | 2.680906149 | 2.575870647 | UP | 20 DPA | Cell wall |
| | | AT4G27110.1) | | | | | |
| Solyc09g010860 | Solyc09g010860.3.1 | expansin precursor 4 | 3.176774194 | 3.448179272 | UP | 20 DPA | Cell wall |
| Solyc03g025600 | Solyc03g025600.3.1 | Pectinacetylesterase family protein (AHRD V3.3 | 1.821187077 | 1.578810248 | UP | 20 DPA | Cell wall |
| | | *** AT5G23870.3) | | | | | |
| Solyc11g005150 | Solyc11g005150.2.1 | entensin X55687 | 1.939546449 | 1.536767149 | UP | 20 DPA | Cell wall |
| Solyc10g080210 | Solyc10g080210.2.1 | polygalacturonase | 14.53389831 | 163.3333333 | UP | 20 DPA | Cell wall |
| Solyc12g040860 | Solyc12g040860.2.1 | Glucan endo-1,3-beta-glucosidase, putative | 0.367623173 | 0.495258935 | DOWN | 6 DPA | Cell wall |
| | | (AHRD V3.3 *** A0A061FDC8_THECC) | | | | | |
| Solyc06g009190 | Solyc06g009190.3.1 | Pectinesterase (AHRD V3.3 *** | 0.5985557 | 0.473231 | DOWN | 6 DPA | Cell wall |
| | | K4C3U9_SOLLC) | | | | | |
| Solyc11g005820 | Solyc11g005820.1.1 | Pectinesterase inhibitor (AHRD V3.3 *** | 0.657661515 | 0.653020448 | DOWN | 6 DPA | Cell wall |
| | | A0A103Y9H8_CYNCS) | | | | | |
| Solyc03g058910 | Solyc03g058910.3.1 | Pectate lyase (AHRD V3.3 *** | 0.114709852 | 0.077554745 | DOWN | 6 DPA | Cell wall |
| | | M1A3P9_SOLTU) | | | | | |
| Solyc06g074630 | Solyc06g074630.3.1 | Cellulose synthase-like protein (AHRD V3.3 *** | 0.644669181 | 0.598504137 | DOWN | 6 DPA | Cell wall |
| | | L0ATP8_POPTO) | | | | | |
| Solyc09g005530 | Solyc09g005530.3.1 | UDP-glucuronic acid decarboxylase 1 (AHRD | 0 | 0 | DOWN | 20 DPA | Cell wall |
| | | V3.3 *—* AT3G53520.4) | | | | | |
| Solyc12g055970 | Solyc12g055970.2.1 | Endoglucanase (AHRD V3.3 *** | 0.304654443 | 0.235294118 | DOWN | 20 DPA | Cell wall |
| | | Q93WY9_TOBAC) | | | | | |
| Solyc12g056960 | Solyc12g056960.2.1 | Glucan 1,3-beta-glucosidase (AHRD V3.3 *** | 0.30551817 | 0.320169252 | DOWN | 20 DPA | Cell wall |
| | | A0A151TZN5_CAJCA) | | | | | |
| Solyc05g005170 | Solyc05g005170.3.1 | Pectin lyase-like superfamily protein (AHRD | 0.335311573 | 0.374792703 | DOWN | 20 DPA | Cell wall |
| | | V3.3 *** A0A061E6B2_THECC) | | | | | |
| Solyc03g058910 | Solyc03g058910.3.1 | Pectate lyase (AHRD V3.3 *** | 0.10041841 | 0.157377049 | DOWN | 20 DPA | Cell wall |
| | | M1A3P9_SOLTU) | | | | | |
| Solyc04g008210 | Solyc04g008210.2.1 | ETAG-A3 | 0.559619238 | 0.545010978 | DOWN | 20 DPA | Cell wall |
| Solyc04g008230 | Solyc04g008230.3.1 | Pectin lyase-like superfamily protein (AHRD | 0.352300351 | 0.541891705 | DOWN | 20 DPA | Cell wall |
| | | V3.3 *** AT1G48100.1) | | | | | |
| Solyc09g092520 | Solyc09g092520.3.1 | xyloglucan endotransglycosylase | 0.523869717 | 0.59311102 | DOWN | 20 DPA | Cell wall |
| Solyc03g119080 | Solyc03g119080.4.1 | beta-mannosidase enzyme | 0.644282941 | 0.657890055 | DOWN | 20 DPA | Cell wall |
| Solyc06g009190 | Solyc06g009190.3.1 | Pectinesterase (AHRD V3.3 *** | 0.487106938 | 0.607263107 | DOWN | 20 DPA | Cell wall |
| | | K4C3U9_SOLLC) | | | | | |
| Solyc04g074830 | Solyc04g074830.1.1 | At4g40080-like protein | 1.667910448 | 1.689581857 | UP | 6 DPA | Cellular trafficking |
| Solyc01g106950 | Solyc01g106950.3.1 | myosin heavy chain-like protein (AHRD V3.3 | 1.912198181 | 1.754315305 | UP | 20 DPA | Cellular trafficking |
| | | *** AT2G14680.3) | | | | | |
| Solyc01g010430 | Solyc01g010430.3.1 | Actin cross-linking protein, putative (AHRD | 1.987230799 | 1.567498497 | UP | 20 DPA | Cellular trafficking |
| | | V3.3 *** A0A061E7B5_THECC) | | | | | |
| Solyc02g069660 | Solyc02g069660.3.1 | Vacuolar protein sorting-associated protein 54 | 1.937256599 | 1.571428571 | UP | 20 DPA | Cellular trafficking |
| | | (AHRD V3.3 *—* A0A118JS09_CYNCS) | | | | | |
| Solyc10g006540 | Solyc10g006540.3.1 | Formin-like protein (AHRD V3.3 *** | 1.963244613 | 1.603519669 | UP | 20 DPA | Cellular trafficking |
| | | K4CXI5_SOLLC) | | | | | |
| Solyc09g064200 | Solyc09g064200.3.1 | Myosin (AHRD V3.3 *** WSZTD6_MAIZE) | 2.149462778 | 1.504768301 | UP | 20 DPA | Cellular trafficking |
| Solyc02g090260 | Solyc02g090260.2.1 | Golgin candidate 6 isoform 2 (AHRD V3.3 *** | 2.472154964 | 1.833034111 | UP | 20 DPA | Cellular trafficking |
| | | A0A061DMU6_THECC) | | | | | |
| Solyc12g010110 | Solyc12g010110.2.1 | Formin-like protein (AHRD V3.3 *** | 2.378099174 | 1.70014771 | UP | 20 DPA | Cellular trafficking |
| | | K4DC98_SOLLC) | | | | | |
| Solyc08g075650 | Solyc08g075650.3.1 | Formin-like protein (AHRD V3.3 *** | 1.953210011 | 1.598871734 | UP | 20 DPA | Cellular trafficking |
| | | K4CMT2_SOLLC) | | | | | |
| Solyc01g081540 | Solyc01g081540.3.1 | Myosin family protein, putative, expressed | 1.857811586 | 1.573534878 | UP | 20 DPA | Cellular trafficking |
| | | (AHRD V3.3 *** Q10CX1_ORYSJ) | | | | | |
| Solyc02g091730 | Solyc02g091730.2.1 | Chromatin SPT2 (AHRD V3.3 *** | 1.57419511 | 2.208149406 | UP | 6 DPA | Chromatin |
| | | A0A118JSU1_CYNCS) | | | | | remodeling |
| Solyc06g071580 | Solyc06g071580.3.1 | MORC family CW-type zinc finger protein 4 | 2.56932966 | 4.455414013 | UP | 6 DPA | Chromatin |
| | | (AHRD V3.3 *** A0A0B0PTV2_GOSAR) | | | | | remodeling |
| Solyc11g062010 | Solyc11g062010.2.1 | Chromodomain-helicase-DNA-binding protein 1 | 1.746601722 | 1.506791752 | UP | 20 DPA | Chromatin |
| | | | | | | | remodeling |
| Solyc09g009080 | Solyc09g009080.3.1 | DNA demethylasel | 1.810532341 | 1.75527192 | UP | 20 DPA | Chromatin |
| | | | | | | | remodeling |
| Solyc09g008520 | Solyc09g008520.3.1 | RING/FYVE/PHD zinc finger protein (AHRD | 1.60664523 | 1.587923729 | UP | 20 DPA | Chromatin |
| | | V3.3 *** A0A072VVJ3_MEDTR) | | | | | remodeling |
| Solyc06g071580 | Solyc06g071580.3.1 | MORC family CW-type zinc finger protein 4 | 3.7347876 | 11.91575092 | UP | 20 DPA | Chromatin |
| | | (AHRD V3.3 *** A0A0B0PTV2_GOSAR) | | | | | remodeling |
| Solyc01g006880 | Solyc01g006880.3.1 | Histone-lysine N-methyltransferase (AHRD V3.3 | 1.998004988 | 1.597288676 | UP | 20 DPA | Chromatin |
| | | *—* A0A0K9P7Q5_ZOSMR) | | | | | remodeling |
| Solyc08g065820 | Solyc08g065820.2.1 | Transcription factor jumonji (jmjC) domain- | 2.200471698 | 1.729379055 | UP | 20 DPA | Chromatin |
| | | containing protein (AHRD V3.3 *—* | | | | | remodeling |
| | | AT3G07610.1) | | | | | |
| Solyc07g052020 | Solyc07g052020.2.1 | SNF2 domain-containing protein / helicase | #DIV/0! | 6.655172414 | UP | 20 DPA | Chromatin |
| | | domain-containing protein (AHRD V3.3 *—* | | | | | remodeling |
| | | AT3G12810.2) | | | | | |
| Solyc12g099910 | Solyc12g099910.2.1 | chromatin remodeling 5 (AHRD V3.3 *** | 1.567672611 | 1.555580762 | UP | 20 DPA | Chromatin |
| | | AT2G13370.3) | | | | | remodeling |
| Solyc06g050320 | Solyc06g050320.3.1 | High mobility group (HMG) box domain- | 0.596166307 | 0.632251045 | DOWN | 6 DPA | Chromatin |
| | | containing protein (AHRD V3.3 *** | | | | | remodeling |
| | | A0A103YDE8_CYNCS) | | | | | |
| Solyc01g006680 | Solyc01g006680.3.1 | Transcription factor jumonji jmjC domain | 0.6562732 | 0.591304348 | DOWN | 20 DPA | Chromatin |
| | | protein | | | | | remodeling |
| Solyc01g087400 | Solyc01g087400.3.1 | Histone-lysine N-methyltransferase MII4 | 1.739947656 | 1.600218818 | UP | 20 DPA | Chromatin |
| | | (AHRD V3.3 *** A0A0B0PKG0_GOSAR) | | | | | remodeling |
| Solyc04g051490 | Solyc04g051490.3.1 | essential meiotic endonuclease 1B (AHRD V3.3 | 1.821969697 | 2.586021505 | UP | 20 DPA | DNA repair |
| | | *—* AT2G22140.1) | | | | | |
| Solyc02g091620 | Solyc02g091620.2.1 | DNA mismatch repair protein-like | 3.071135431 | 2.388297872 | UP | 20 DPA | DNA repair |
| Solyc05g051680 | Solyc05g051680.3.1 | Structural maintenance of chromosomes family | 1.767430025 | 1.796689084 | UP | 20 DPA | DNA repair |
| | | protein (AHRD V3.3 *** D7M0L2_ARALL) | | | | | |
| Solyc12g042000 | Solyc12g042000.2.1 | Protein BREAST CANCER SUSCEPTIBILITY | 3.879882093 | 2.119565217 | UP | 20 DPA | DNA repair |
| | | 1 like (AHRD V3.3 *—* | | | | | |
| | | A0A0B2PNV8_GLYSO) | | | | | |
| Solyc09g066080 | Solyc09g066080.2.1 | Protein BREAST CANCER SUSCEPTIBILITY | 2.148946595 | 1.570916905 | UP | 20 DPA | DNA repair |
| | | 1-like protein (AHRD V3.3 *—* | | | | | |
| | | W9R323_9ROSA) | | | | | |
| Solyc07g052010 | Solyc07g052010.2.1 | SNF2 domain-containing protein (AHRD V3.3 | 13.92857143 | 8.125 | UP | 20 DPA | DNA repair |
| | | *** D7KEB6_ARALL) | | | | | |
| Solyc01g098520 | Solyc01g098520.3.1 | DNA mismatch repair protein mutL (AHRD | 2.139931741 | 1.628571429 | UP | 20 DPA | DNA repair |
| | | V3.3 *—* A0A151THR9_CAJCA) | | | | | |
| Solyc10g006200 | Solyc10g006200.3.1 | Ribonuclease 3-like protein 2 (AHRD V3.3 *** | 2.343592965 | 1.79375 | UP | 20 DPA | Gene |
| | | A0A061F5X1_THECC) | | | | | silencing/siRNA |
| | | | | | | | producion |
| Solyc11g008530 | Solyc11g008530.2.1 | Dicer-like 2d | 1.662682927 | 1.607024988 | UP | 20 DPA | Gene |
| | | | | | | | silencing/siRNA |
| | | | | | | | producion |
| Solyc04g082630 | Solyc04g082630.3.1 | Glyceraldehyde-3-phosphate dehydrogenase | 0.50319231 | 0.667224858 | DOWN | 20 DPA | Glycolysis |
| | | (AHRD V3.3 *** K4BVZ0_SOLLC) | | | | | |
| Solyc04g009030 | Solyc04g009030.3.1 | Glyceraldehyde-3-phosphate dehydrogenase | 0.6181287 | 0.657776323 | DOWN | 20 DPA | Glycolysis |
| | | (AHRD V3.3 *** K4BP59_SOLLC) | | | | | |
| Solyc09g059170 | Solyc09g059170.2.1 | Glycosyltransferase (AHRD V3.3 *** | 0.393763214 | 0.413028413 | DOWN | 20 DPA | Glycosylation |
| | | M1C989_SOLTU) | | | | | |
| Solyc12g098600 | Solyc12g098600.1.1 | Glycosyltransferase (AHRD V3.3 *** | 0.602474449 | 0.583789419 | DOWN | 20 DPA | Glycosylation |
| | | K4DHN3_SOLLC) | | | | | |
| Solyc09g092500 | Solyc09g092500.1.1 | Glycosyltransferase (AHRD V3.3 *** | 0.650985317 | 0.54067715 | DOWN | 20 DPA | Glycosylation |
| | | K4CWS6_SOLLC) | | | | | |
| Solyc07g043500 | Solyc07g043500.1.1 | Glycosyltransferase (AHRD V3.3 *** | 0.476422404 | 0.374586597 | DOWN | 20 DPA | Glycosylation |
| | | K4CEK8_SOLLC) | | | | | |
| Solyc03g116530 | Solyc03g116530.3.1 | Lactoylglutathione lyase / glyoxalase I family | 0.619135518 | 0.458584938 | DOWN | 20 DPA | Glyoxalase pathway |
| | | protein (AHRD V3.3 *** AT1G80160.3) | | | | | |
| Solyc01g103590 | Solyc01g103590.3.1 | Lactoylglutathione lyase / glyoxalase I family | 0.282135524 | 0.313412409 | DOWN | 20 DPA | Glyoxalase pathway |
| | | protein (AHRD V3.3 *** | | | | | |
| | | A0A061DWL3_THECC) | | | | | |
| Solyc07g044750 | Solyc07g044750.3.1 | N-acetyl-D-glucosamine kinase (AHRD V3.3 | 1.946481665 | 1.921722114 | UP | 6 DPA | Metabolism/Amino- |
| | | *** A0A0B0P7N8_GOSAR) | | | | | sugar |
| Solyc02g093430 | Solyc02g093430.3.1 | Beta-1,4-N-acetylglucosaminyltransferase-like | 1.938682551 | 1.723676012 | UP | 6 DPA | Metabolism/Amino- |
| | | protein (AHRD V3.3 *** | | | | | sugar |
| | | A0A072U8D1_MEDTR) | | | | | |
| Solyc12g008900 | Solyc12g008900.2.1 | cytokinin oxidase6 | 5.77124183 | 7.483050847 | UP | 20 DPA | Metabolism/Cytokinin |
| Solyc02g091990 | Solyc02g091990.3.1 | 1-aminocyclopropane-1-carboxylate synthase 3 | 2.124171187 | 2.631814787 | UP | 6 DPA | Metabolism/Ethylene |
| Solyc07g049530 | Solyc07g049530.3.1 | 1-aminocyclopropane-1-carboxylate oxidase 1 | 1.808275198 | 3.122974657 | UP | 20 DPA | Metabolism/Ethylene |
| Solyc04g049360 | Solyc04g049360.3.1 | aminodeoxychorismate synthase/glutamine | 2.429983526 | 1.855345912 | UP | 20 DPA | Metabolism/Folate |
| | | amidotransferase | | | | | |
| Solyc03g006880 | Solyc03g006880.3.1 | gibberellin 20-oxidase-1 | 0.609409856 | 0.513499658 | DOWN | 20 DPA | Metabolism/Gibberellin |
| Solyc02g070430 | Solyc02g070430.3.1 | Gibberellin 2 oxidase (AHRD V3.3 *** | 0.283244681 | 0.241770715 | DOWN | 20 DPA | Metabolism/Gibberellin |
| | | Q53C60_NEROL) | | | | | |
| Solyc08g083110 | Solyc08g083110.3.1 | Cystathionine gamma-synthase, putative (AHRD | 2.235622817 | 1.624703892 | UP | 6 DPA | Metabolism/Amino |
| | | V3.3 *—* B9RYU1_RICCO) | | | | | acids |
| Solyc06g050630 | Solyc06g050630.3.1 | arogenate dehydrogenase (AHRD V3.3 *—* | 2.589939024 | 4.732590529 | UP | 6 DPA | Metabolism/Amino |
| | | AT5G34930.3) | | | | | acids |
| Solyc06g011530 | Solyc06g011530.3.1 | Hydroxymethylglutaryl-CoA lyase (AHRD V3.3 | 1.890455095 | 1.66749556 | UP | 20 DPA | Metabolism/Amino |
| | | *—* W9QRW1_9ROSA) | | | | | acids |
| Solyc07g017670 | Solyc07g017670.2.1 | Lysine-ketoglutarate reductase/saccharopine | 4.587163233 | 3.828703704 | UP | 20 DPA | Metabolism/Amino |
| | | dehydrogenase (AHRD V3.3 *—* | | | | | acids |
| | | A0A072V2T5_MEDTR) | | | | | |
| Solyc08g068600 | Solyc08g068600.3.1 | Aromatic amino acid decarboxylase 1B (AHRD | 1.755698333 | 1.538059501 | UP | 6 DPA | Metabolism/lipid |
| | | V3.3 *** Q1KSC5_SOLLC) | | | | | |
| Solyc02g094400 | Solyc02g094400.3.1 | Glycerophosphodiester phosphodiesterase, | 1.891148525 | 1.763837065 | UP | 6 DPA | Metabolism/Lipid |
| | | putative (AHRD V3.3 *** B9RH28_RICCO) | | | | | |
| Solyc03g115370 | Solyc03g115370.3.1 | Diacylglycerol kinase (AHRD V3.3 *** | 1.700235571 | 2.023125438 | UP | 6 DPA | Metabolism/Lipid |
| | | K4BL39_SOLLC) | | | | | |
| Solyc10g086690 | Solyc10g086690.2.1 | Phosphatidylinositol: ceramide | 1.860497238 | 2.748979592 | UP | 6 DPA | Metabolism/Lipid |
| | | inositolphosphotransferase (AHRD V3.3 *** | | | | | |
| | | IPCS_ORYSI) | | | | | |
| Solyc02g036290 | Solyc02g036290.3.1 | malonyl-CoA decarboxylase family protein | 1.76147541 | 1.960766423 | UP | 20 DPA | Metabolism/Lipid |
| | | (AHRD V3.3 *—* AT4G04320.2) | | | | | |
| Solyc03g044910 | Solyc03g044910.1.1 | GNS1/SUR4 membrane protein family (AHRD | 1.678817056 | 1.543308746 | UP | 20 DPA | Metabolism/Lipid |
| | | V3.3 *** AT4G36830.1) | | | | | |
| Solyc07g032180 | Solyc07g032180.3.1 | Abhydrolase domain-containing protein 4 | 3.23032491 | 1.641232575 | UP | 20 DPA | Metabolism/Lipid |
| | | (AHRD V3.3 *** A0A0B2SN17_GLYSO) | | | | | |
| Solyc09g075790 | Solyc09g075790.3.1 | Long-Chain Acyl-CoA Synthetase (AHRD V3.3 | 2.800166528 | 1.978235294 | UP | 20 DPA | Metabolism/Lipid |
| | | *** A0A0R5QM74_SALMI) | | | | | |
| Solyc07g032170 | Solyc07g032170.3.1 | Abhydrolase domain-containing protein | 3.223153693 | 2.155365723 | UP | 20 DPA | Metabolism/Lipid |
| Solyc07g054830 | Solyc07g054830.3.1 | diacylglycerol kinase | 1.643932684 | 1.500404204 | UP | 20 DPA | Metabolism/Lipid |
| Solyc02g082770 | Solyc02g082770.3.1 | Phosphatidylinositide phosphatase SAC1 | 2.655472363 | 1.753708551 | UP | 20 DPA | Metabolism/Lipid |
| Solyc05g042070 | Solyc05g042070.3.1 | 1-phosphatidylinositol-4-phosphate 5-kinase 3 | 2.133703825 | 2.020117611 | UP | 20 DPA | Metabolism/Lipid |
| | | (AHRD V3.3 ——* AT2G26420.3) | | | | | |
| Solyc06g030590 | Solyc06g030590.2.1 | 8-amino-7-oxononanoate synthase | 14.24 | 6.592592593 | UP | 20 DPA | Metabolism/Lipid |
| Solyc08g082920 | Solyc08g082920.3.1 | ORMDL family protein (AHRD V3.3 *** | 0.660786366 | 0.668582665 | DOWN | 6 DPA | Metabolism/Lipid |
| | | AT5G42000.1) | | | | | |
| Solyc07g014730 | Solyc07g014730.3.1 | Phospholipase A2 (AHRD V3.3 *** | 0.459466139 | 0.486776643 | DOWN | 20 DPA | Metabolism/Lipid |
| | | Q5CCT8_TOBAC) | | | | | |
| Solyc01g005930 | Solyc01g005930.3.1 | Lipase/lipooxygenase, PLAT/LH2 (AHRD V3.3 | 0.573055028 | 0.516902011 | DOWN | 20 DPA | Metabolism/Lipid |
| | | *** A0A118JU91_CYNCS) | | | | | |
| Solyc06g068010 | Solyc06g068010.3.1 | Biotin carboxyl carrier protein (AHRD V3.3 *** | 0.548246885 | 0.510248112 | DOWN | 20 DPA | Metabolism/Lipid |
| | | A0A0H5BV69_PEA) | | | | | |
| Solyc07g056320 | Solyc07g056320.3.1 | Glycerol-3-phosphate acyltransferase (AHRD | 0.35976016 | 0.494505495 | DOWN | 20 DPA | Metabolism/Lipid |
| | | V3.3 *** G7K3C7_MEDTR) | | | | | |
| Solyc06g069640 | Solyc06g069640.3.1 | dCTP pyrophosphatase 1 (AHRD V3.3 *** | 0.513905831 | 0.498697917 | DOWN | 20 DPA | Metabolism/Nucleotide |
| | | A0A151SL49_CAJCA) | | | | | |
| Solyc06g074710 | Solyc06g074710.1.1 | hydroxycinnamoyl-CoA shikimate/quinate | 0.464136092 | 0.531781818 | DOWN | 20 DPA | Metabolism/ |
| | | hydroxycinnamoyl transferase (AHRD V3.3 *** | | | | | Phenylpropanoids |
| | | AT5G48930.1) | | | | | |
| Solyc04g054260 | Solyc04g054260.3.1 | Cytochrome P450 family protein (AHRD V3.3 | 0.144254279 | 0.207017544 | DOWN | 20 DPA | Metabolism/ |
| | | *** B9HFW5_POPTR) | | | | | Phenylpropanoids |
| Solyc06g068440 | Solyc06g068440.3.1 | cinnamoyl-CoA reductase | 0.662819456 | 0.619017067 | DOWN | 20 DPA | Metabolism/ |
| | | | | | | | Phenylpropanoids |
| Solyc01g081050 | Solyc01g081050.4.1 | D-alanine--poly(phosphoribitol) ligase subunit 1 | 2.796927188 | 1.628549203 | UP | 20 DPA | Metabolism/RNA |
| | | (AHRD V1 *——— DLTA_BACSU)%3B contains | | | | | |
| | | Interpro domain(s) IPR000873 | | | | | |
| Solyc08g066650 | Solyc08g066650.3.1 | Carotenoid Cleavage Dioxygenase 8 | 0.400986031 | 0.315857605 | DOWN | 20 DPA | Metabolism/ |
| | | | | | | | Strigolactone |
| Solyc08g082440 | Solyc08g082440.3.1 | UDP-glucose 4-epimerase (AHRD V3.3 *** | 0.639279438 | 0.581302437 | DOWN | 20 DPA | Metabolism/Sugar |
| | | Q6XZA0_SOLTU) | | | | | |
| Solyc07g064160 | Solyc07g064160.3.1 | Thiamine thiazole synthase, chloroplastic | 0.591909213 | 0.396333686 | DOWN | 20 DPA | Metabolism/Thiamine |
| | | (AHRD V3.3 *** K4CH99_SOLLC) | | | | | |
| Solyc10g018300 | Solyc10g018300.2.1 | Transketolase (AHRD V3.3 *** AT3G60750.1) | 0.52392963 | 0.593076374 | DOWN | 20 DPA | Pentose phosphate |
| | | | | | | | pathway |
| Solyc03g115820 | Solyc03g115820.3.1 | Ribulose-phosphate 3-epimerase (AHRD V3.3 | 0.637219731 | 0.591590341 | DOWN | 20 DPA | Pentose phosphate |
| | | *** A0A072V1G6_MEDTR) | | | | | pathway |
| Solyc06g054220 | Solyc06g054220.2.1 | Photosystem II reaction center PsbP family | 2.715497301 | 2.537463977 | UP | 20 DPA | Photosynthesis |
| | | protein (AHRD V3.3 ——* AT4G15510.1) | | | | | |
| Solyc00g230080 | Solyc00g230080.1.1 | LOW QUALITY: Photosystem II D2 protein | 54.63636364 | 6.326315789 | UP | 20 DPA | Photosynthesis |
| | | (AHRD V3.3 *—* PSBD_PLETE) | | | | | |
| Solyc02g069450 | Solyc02g069450.3.1 | Photosystem I reaction center subunit III (AHRD | 0.347567312 | 0.631696429 | DOWN | 20 DPA | Photosynthesis |
| | | V3.3 *** 13SN70_MEDTR) | | | | | |
| Solyc06g074200 | Solyc06g074200.3.1 | photosystem I subunit O (AHRD V3.3 *** | 0.407573526 | 0.606689696 | DOWN | 20 DPA | Photosynthesis |
| | | AT1G08380.1) | | | | | |
| Solyc06g083680 | Solyc06g083680.3.1 | Photosystem I reaction center subunit IV (AHRD | 0.438852215 | 0.669402247 | DOWN | 20 DPA | Photosynthesis |
| | | V3.3 *** A0A0K9P6J8_ZOSMR) | | | | | |
| Solyc10g006370 | Solyc10g006370.3.1 | Lhcp translocation defect-like protein (AHRD | 0.53268945 | 0.65014357 | DOWN | 20 DPA | Photosynthesis |
| | | V3.3 *** A0A0B0PFC4_GOSAR) | | | | | |
| Solyc08g006930 | Solyc08g006930.3.1 | Photosystem I reaction center subunit psaK | 0.565051353 | 0.641728273 | DOWN | 20 DPA | Photosynthesis |
| | | (AHRD V3.3 *** W9SCM1_9ROSA) | | | | | |
| Solyc07g065200 | Solyc07g065200.3.1 | Mitochondrial import inner membrane | 0.606244685 | 0.656233561 | DOWN | 20 DPA | Photosynthesis |
| | | translocase subunit tim22 (AHRD V3.3 *** | | | | | |
| | | A0A0B0NT47_GOSAR) | | | | | |
| Solyc03g034220 | Solyc03g034220.3.1 | Tomato RuBP carboxylase small subunit | 0.605714146 | 0.57717745 | DOWN | 20 DPA | Photosynthesis |
| Solyc10g077040 | Solyc10g077040.2.1 | putative magnesium-protoporphyrin monomethyl | 0.563306206 | 0.65136675 | DOWN | 20 DPA | Photosynthesis |
| | | ester cyclase | | | | | |
| Solyc03g005760 | Solyc03g005760.1.1 | chlorophyll a/b-binding protein | 0.505873923 | 0.607866064 | DOWN | 20 DPA | Photosynthesis |
| Solyc02g071030 | Solyc02g071030.2.1 | Chlorophyll a-b binding protein, chloroplastic | 0.380725582 | 0.594535543 | DOWN | 20 DPA | Photosynthesis |
| | | (AHRD V3.3 *** Q41422_SOLTU) | | | | | |
| Solyc03g005780 | Solyc03g005780.3.1 | chlorophyll a/b-binding protein Cab-3C | 0.290474466 | 0.453876627 | DOWN | 20 DPA | Photosynthesis |
| Solyc01g105050 | Solyc01g105050.3.1 | Chlorophyll a-b binding protein, chloroplastic | 0.432724108 | 0.552567237 | DOWN | 20 DPA | Photosynthesis |
| | | (AHRD V3.3 *** M1AY18_SOLTU) | | | | | |
| Solyc03g005770 | Solyc03g005770.3.1 | chlorophyll a/b-binding protein | 0.53302625 | 0.569032909 | DOWN | 20 DPA | Photosynthesis |
| Solyc03g112340 | Solyc03g112340.1.1 | RING/U-box superfamily protein (AHRD V3.3 | 1.500472441 | 1.676284307 | UP | 6 DPA | Protein degradation |
| | | *** AT3G05200.1) | | | | | |
| Solyc04g082420 | Solyc04g082420.3.1 | BTB/POZ domain-containing protein (AHRD | 1.712795549 | 1.813696613 | UP | 6 DPA | Protein degradation |
| | | V3.3 *** AT2G30600.6) | | | | | |
| Solyc04g005040 | Solyc04g005040.1.1 | Matrix metalloproteinase (AHRD V3.3 *** | 1.508605127 | 1.669330137 | UP | 6 DPA | Protein degradation |
| | | I7KJ40_SOLLC) | | | | | |
| Solyc01g106820 | Solyc01g106820.3.1 | Peptidase M50 family | 1.680029696 | 2.628339141 | UP | 6 DPA | Protein degradation |
| Solyc07g043510 | Solyc07g043510.3.1 | OTU domain-containing (AHRD V3.3 *** | 1.751452282 | 1.676330421 | UP | 6 DPA | protein degradation |
| | | A0A0B0PSP5_GOSAR) | | | | | |
| Solyc04g008100 | Solyc04g008100.2.1 | U-box domain-containing protein (AHRD V3.3 | 2.491947291 | 2.30936228 | UP | 6 DPA | Protein degradation |
| | | *** A0A0A0RAL4_9ROSI) | | | | | |
| Solyc11g068710 | Solyc11g068710.2.1 | F-box family protein (AHRD V3.3 *** | 1.594758621 | 1.529567403 | UP | 6 DPA | Protein degradation |
| | | B9MYU0_POPTR) | | | | | |
| Solyc03g117860 | Solyc03g117860.3.1 | RING/U-box superfamily protein (AHRD V3.3 | 2.351815824 | 2.889641434 | UP | 20 DPA | protein degradation |
| | | *—* AT3G14250.1) | | | | | |
| Solyc11g005650 | Solyc11g005650.1.1 | Ubiquitin family protein (AHRD V3.3 *—* | 2.378167641 | 2.513736264 | UP | 20 DPA | Protein degradation |
| | | AT4G02890.4) | | | | | |
| Solyc11g005640 | Solyc11g005640.2.1 | Polyubiquitin (AHRD V3.3 *—* UBI2P_PETCR) | 1.839530333 | 2.30958231 | UP | 20 DPA | Protein degradation |
| Solyc02g067260 | Solyc02g067260.2.1 | RING/U-box superfamily protein (AHRD V3.3 | 2.705641864 | 3.250491159 | UP | 20 DPA | Protein degradation |
| | | *—* AT1G26800.1) | | | | | |
| Solyc02g014170 | Solyc02g014170.3.1 | RING/U-box superfamily protein (AHRD V3.3 ——* | 1.832647462 | 1.652442795 | UP | 20 DPA | Protein degradation |
| | | AT2G38195.1) | | | | | |
| Solyc07g007170 | Solyc07g007170.3.1 | RING/U-box superfamily protein (AHRD V3.3 | 1.654096339 | 1.96 | UP | 20 DPA | Protein degradation |
| | | *** A0A061GN77_THECC) | | | | | |
| Solyc11g020960 | Solyc11g020960.2.1 | Proteinase inhibitor II (AHRD V3.3 *—* | 2.266082034 | 1.902516309 | UP | 20 DPA | Protein degradation |
| | | B3F0C1_TOBAC) | | | | | |
| Solyc07g008430 | Solyc07g008430.2.1 | RING/U-box superfamily protein (AHRD V3.3 ——* | 0.416714404 | 0.511664257 | DOWN | 20 DPA | Protein degradation |
| | | AT3G56580.3) | | | | | |
| Solyc11g066510 | Solyc11g066510.2.1 | RING/U-box superfamily protein (AHRD V3.3 | 0.487200161 | 0.575339205 | DOWN | 20 DPA | Protein degradation |
| | | *** AT2G27940.1) | | | | | |
| Solyc04g079780 | Solyc04g079780.3.1 | RING/U-box superfamily protein (AHRD V3.3 | 0.659682247 | 0.647750396 | DOWN | 20 DPA | Protein degradation |
| | | *** AT2G16090.1) | | | | | |
| Solyc07g049140 | Solyc07g049140.3.1 | Metallocarboxypeptidase inhibitor (AHRD V3.3 | 0.663099208 | 0.544436272 | DOWN | 20 DPA | Protein degradation |
| | | *** O24639_SOLTU) | | | | | |
| Solyc01g096450 | Solyc01g096450.3.1 | Aspartic proteinase nepenthesin-1-like protein | 0.485988387 | 0.633119553 | DOWN | 20 DPA | Protein degradation |
| | | (AHRD V3.3 *** T2DNA5_PHAVU) | | | | | |
| Solyc12g056410 | Solyc12g056410.2.1 | F-box protein PP2-A13 (AHRD V3.3 *** | 0.651685393 | 0.635077519 | DOWN | 20 DPA | Protein degradation |
| | | P2A13_ARATH) | | | | | |
| Solyc02g069110 | Solyc02g069110.3.1 | Cathepsin B-like cysteine protease (AHRD V3.3 | 0.566188198 | 0.479915025 | DOWN | 20 DPA | Protein degradation |
| | | *** B7FJ05_MEDTR) | | | | | |
| Solyc09g075080 | Solyc09g075080.3.1 | Phytochrome A-associated F-box protein, | 0.534989648 | 0.515974441 | DOWN | 20 DPA | Protein degradation |
| | | putative (AHRD V3.3 *** B9SAQ8_RICCO) | | | | | |
| Solyc05g041860 | Solyc05g041860.3.1 | N-acetyltransferase (AHRD V3.3 *** | 2.106119926 | 1.804873742 | UP | 20 DPA | Protein modification |
| | | B6TRH8_MAIZE) | | | | | |
| Solyc12g062340 | Solyc12g062340.2.1 | S-adenosyl-L-methionine-dependent | 9.468085106 | 6.267605634 | UP | 20 DPA | Protein modification |
| | | methyltransferases superfamily protein (AHRD | | | | | |
| | | V3.3 ——* AT3G44840.1) | | | | | |
| Solyc04g072260 | Solyc04g072260.2.1 | Midasin (AHRD V3.3 *—* K4BTL3_SOLLC) | 2.013583138 | 1.854616048 | UP | 20 DPA | Ribosome biogenesis |
| Solyc01g104470 | Solyc01g104470.3.1 | Acidic ribosomal protein PO (AHRD V3.3 *** | 0.66043877 | 0.611390435 | DOWN | 20 DPA | Ribosome biogenesis |
| | | B910W5_POPTR) | | | | | |
| Solyc04g081330 | Solyc04g081330.3.1 | 30S ribosomal protein S16, chloroplastic (AHRD | 0.665914221 | 0.664663913 | DOWN | 20 DPA | Ribosome biogenesis |
| | | V3.3 ——* RR16_MORIN) | | | | | |
| Solyc04g079790 | Solyc04g079790.3.1 | 30S ribosomal protein S9 (AHRD V3.3 *** | 0.663109049 | 0.653929756 | DOWN | 20 DPA | Ribosome biogenesis |
| | | A9PB01_POPTR) | | | | | |
| Solyc03g081300 | Solyc03g081300.3.1 | Mitochondrial transcription termination factor- | 1.537971014 | 1.685514612 | UP | 6 DPA | RNA processing |
| | | related family protein (AHRD V3.3 *** | | | | | |
| | | B9IDI0_POPTR) | | | | | |
| Solyc02g084330 | Solyc02g084330.3.1 | decapping 5-like protein (AHRD V3.3 ——* | 2.805687204 | 2.524520256 | UP | 20 DPA | RNA processing |
| | | AT5G45330.3) | | | | | |
| Solyc05g009540 | Solyc05g009540.3.1 | Protein kinase-like protein (AHRD V3.3 *** | 1.675944334 | 1.581613508 | UP | 20 DPA | RNA processing |
| | | Q8RWN3_ARATH) | | | | | |
| Solyc06g076850 | Solyc06g076850.3.1 | Tetratricopeptide repeat (TPR)-like superfamily | 1.675384392 | 1.524694013 | UP | 20 DPA | RNA processing |
| | | protein (AHRD V3.3 *** AT2G29670.2) | | | | | |
| Solyc01g087320 | Solyc01g087320.3.1 | tRNA-dihydrouridine synthase A | 1.88104724 | 1.833056018 | UP | 20 DPA | RNA processing |
| Solyc11g008470 | Solyc11g008470.2.1 | U4/U6.U5 tri-snRNP-associated protein 1 | 1.55006105 | 1.611722387 | UP | 20 DPA | RNA processing |
| | | (AHRD V3.3 *** A0A0B2S142_GLYSO) | | | | | |
| Solyc11g022600 | Solyc11g022600.2.1 | Mitochondrial transcription termination factor- | 1.745073109 | 1.654278827 | UP | 20 DPA | RNA processing |
| | | like (AHRD V3.3 *—* Q6Z8L0_ORYSJ) | | | | | |
| Solyc01g090860 | Solyc01g090860.3.1 | Nucleotidyltransferase family protein (AHRD | 2.043956044 | 1.564120533 | UP | 20 DPA | RNA processing |
| | | V3.3 *** AT4G00060.4) | | | | | |
| Solyc11g039950 | Solyc11g039950.2.1 | Splicing factor 3B subunit 4 | 2.296890424 | 1.871129875 | UP | 20 DPA | RNA processing |
| Solyc04g050760 | Solyc04g050760.3.1 | U11/U12 small nuclear ribonucleoprotein | 2.014789534 | 1.690692124 | UP | 20 DPA | RNA processing |
| | | (AHRD V3.3 *** AT3G04160.3) | | | | | |
| Solyc08g077310 | Solyc08g077310.3.1 | RNA-binding protein, putative (AHRD V3.3 *** | 1.51488 | 1.525620367 | UP | 20 DPA | RNA processing |
| | | A0A072U387_MEDTR) | | | | | |
| Solyc02g086600 | Solyc02g086600.3.1 | polyribonucleotide nucleotidyltransferase | 1.651977401 | 1.558635394 | UP | 20 DPA | RNA processing |
| | | (AHRD V3.3 *** AT5G14580.1) | | | | | |
| Solyc05g013400 | Solyc05g013400.3.1 | Pentatricopeptide repeat superfamily protein, | 1.770477816 | 1.658673062 | UP | 20 DPA | RNA processing |
| | | putative (AHRD V3.3 *** | | | | | |
| | | A0A061GR61_THECC) | | | | | |
| Solyc03g063370 | Solyc03g063370.3.1 | Pentatricopeptide repeat-containing protein | 6.605381166 | 3.024640657 | UP | 20 DPA | RNA processing |
| | | (AHRD V3.3 *—* A0A0B2RWF1_GLYSO) | | | | | |
| Solyc04g057990 | Solyc04g057990.3.1 | mRNA 3&apos-end-processing protein YTH1 | 2.365453248 | 2.221179625 | UP | 20 DPA | RNA processing |
| Solyc07g041020 | Solyc07g041020.3.1 | RNA-binding protein (AHRD V3.3 *** | 0.629372497 | 0.634967672 | DOWN | 6 DPA | RNA processing |
| | | B4FIJ2_MAIZE) | | | | | |
| Solyc10g050450 | Solyc10g050450.2.1 | CSL zinc finger protein, putative (AHRD V3.3 | 0.337520481 | 0.340495868 | DOWN | 20 DPA | RNA processing |
| | | *** G7JEX8_MEDTR) | | | | | |
| Solyc01g028800 | Solyc01g028800.2.1 | RNA binding protein, putative (AHRD V3.3 *** | 0.135483871 | 0.168 | DOWN | 20 DPA | RNA processing |
| | | B9SBN7_RICCO) | | | | | |
| Solyc03g058190 | Solyc03g058190.3.1 | elongation factor family protein (AHRD V3.3 | 1.71155034 | 1.522457763 | UP | 20 DPA | RNA processing |
| | | *** AT5G13650.2) | | | | | |
| Solyc03g113490 | Solyc03g113490.3.1 | HIPL1-like protein (AHRD V3.3 *** | 4.25 | 4.25 | UP | 6 DPA | Signaling |
| | | A0A0B0MLG6_GOSAR) | | | | | |
| Solyc01g096340 | Solyc01g096340.3.1 | Small auxin up-regulated RNA2 | 0.529204499 | 0.478612575 | DOWN | 20 DPA | Signaling/auxin |
| Solyc11g071760 | Solyc11g071760.3.1 | Calmodulin-like protein | 3.099150142 | 2.912673056 | UP | 6 DPA | Signaling/calcium |
| | | | | | | | signaling |
| Solyc02g094000 | Solyc02g094000.1.1 | Calcium-binding protein (AHRD V3.3 *** | 2.249836779 | 1.833433831 | UP | 6 DPA | Signaling/calcium |
| | | A0A199V9T9_ANACO) | | | | | signaling |
| Solyc11g071740 | Solyc11g071740.2.1 | Calmodulin-like protein (AHRD V1 ***— | 2.158940397 | 2.391015356 | UP | 6 DPA | Signaling/calcium |
| | | Q0VJ70_DATME) | | | | | signaling |
| Solyc11g071750 | Solyc11g071750.2.1 | Calmodulin-like protein (AHRD V1 *** | 2.594620104 | 3.154331612 | UP | 6 DPA | Signaling/calcium |
| | | Q0VJ70_DATME) | | | | | signaling |
| Solyc07g056560 | Solyc07g056560.3.1 | cytokinin response factor 2 (AHRD V3.3 ——* | 0.229086229 | 0.26969697 | DOWN | 20 DPA | Signaling/Cytokinin |
| | | AT4G23750.2) | | | | | |
| Solyc10g079700 | Solyc10g079700.2.1 | Two-component response regulator (AHRD V3.3 | 0.454265949 | 0.591295648 | DOWN | 6 DPA | Signaling/Cytokinin |
| | | *** A0A059T9P7_ROSCN) | | | | | |
| Solyc07g064380 | Solyc07g064380.3.1 | serine/threonine-protein phosphatase 7 long | 206.6666667 | 44.28571429 | UP | 20 DPA | Signaling/Kinase |
| | | form-like protein (AHRD V3.3 *—* | | | | | |
| | | AT1G32120.1) | | | | | |
| Solyc02g089010 | Solyc02g089010.2.1 | Receptor protein kinase, putative (AHRD V3.3 | 4.50174703 | 1.970030581 | UP | 20 DPA | Signaling/kinase |
| | | *—* B9RXG1_RICCO) | | | | | |
| Solyc05g013300 | Solyc05g013300.1.1 | Pseudomonas syringae pv tomato resis. | 1.912342328 | 1.519923858 | UP | 20 DPA | Signaling/kinase |
| Solyc02g071820 | Solyc02g071820.3.1 | Receptor-like protein kinase (AHRD V3.3 *** | 2.835045971 | 2.558321132 | UP | 20 DPA | Signaling/kinase |
| | | B9I1R1_POPTR) | | | | | |
| Solyc12g006980 | Solyc12g006980.1.1 | Leucine-rich repeat receptor-like protein kinase | 2.304904133 | 1.806471891 | UP | 20 DPA | Signaling/Kinase |
| | | family (AHRD V3.3 *—* | | | | | |
| | | A0A0K9PTR8_ZOSMR) | | | | | |
| Solyc08g077630 | Solyc08g077630.3.1 | Kinase family protein (AHRD V3.3 *** | 2.173628174 | 1.739187418 | UP | 20 DPA | Signaling/Kinase |
| | | B9MTV7_POPTR) | | | | | |
| Solyc11g061720 | Solyc11g061720.1.1 | Kinase family protein (AHRD V3.3 *** | 0.584471843 | 0.514067995 | DOWN | 20 DPA | Signaling/kinase |
| | | D7KZX1_ARALL) | | | | | |
| Solyc05g005810 | Solyc05g005810.3.1 | Serine/threonine-protein kinase WNK-related | 0.528430371 | 0.575240919 | DOWN | 20 DPA | Signaling/kinase |
| | | (AHRD V3.3 *** A0A061E6Z5_THECC) | | | | | |
| Solyc02g084600 | Solyc02g084600.3.1 | Leucine-rich repeat receptor-like protein kinase | 0.41943271 | 0.443098502 | DOWN | 20 DPA | Signaling/kinase |
| | | family protein (AHRD V3.3 *** | | | | | |
| | | A0A061DFG6_THECC) | | | | | |
| Solyc01g080770 | Solyc01g080770.3.1 | Non-specific serine/threonine protein kinase | 0.44885971 | 0.614765736 | DOWN | 20 DPA | Signaling/kinase |
| | | (AHRD V3.3 *** K4AXX0_SOLLC) | | | | | |
| Solyc05g052520 | Solyc05g052520.3.1 | Protein phosphatase 2C family protein (AHRD | 1.640196078 | 1.846170823 | UP | 6 DPA | Signaling/Protein |
| | | V3.3 *** AT1G07160.1) | | | | | phosphatase |
| Solyc07g064380 | Solyc07g064380.3.1 | serine/threonine-protein phosphatase 7 long | 76.41666667 | 101.8888889 | UP | 6 DPA | Signaling/Protein |
| | | form-like protein (AHRD V3.3 *—* | | | | | phosphatase |
| | | AT1G32120.1) | | | | | |
| Solyc12g010450 | Solyc12g010450.2.1 | Protein phosphatase 2c, putative (AHRD V3.3 | 0.541573868 | 0.613540791 | DOWN | 20 DPA | Signaling/Protein |
| | | *** B9T2N5_RICCO) | | | | | phosphatase |
| Solyc01g007030 | Solyc01g007030.3.1 | U-box domain-containing family protein (AHRD | 2.445914397 | 2.641176471 | UP | 6 DPA | Stress responses |
| | | V3.3 *** B9P691_POPTR) | | | | | |
| Solyc02g087210 | Solyc02g087210.3.1 | Zinc finger AN1 domain-containing stress- | 1.769731632 | 2.136498832 | UP | 6 DPA | Stress responses |
| | | associated protein 12 (AHRD V3.3 *** | | | | | |
| | | A0A151RAZ5_CAJCA) | | | | | |
| Solyc01g109920 | Solyc01g109920.2.1 | Dehydrin protein | 1.949245542 | 1.869736842 | UP | 6 DPA | Stress responses |
| Solyc04g007580 | Solyc04g007580.1.1 | cDNA clone J100026I16 full insert sequence | 1.881307747 | 1.665565518 | UP | 6 DPA | Stress responses |
| Solyc09g011030 | Solyc09g011030.3.1 | Hsp70-binding protein 1 (AHRD V3.3 *** | 1.540306701 | 1.661774173 | UP | 6 DPA | Stress responses |
| | | W9RWP5_9ROSA) | | | | | |
| Solyc05g006860 | Solyc05g006860.3.1 | Thioredoxin family protein (AHRD V3.3 *** | 1.692465402 | 2.742524917 | UP | 6 DPA | Stress responses |
| | | B9IJS4_POPTR) | | | | | |
| Solyc03g080150 | Solyc03g080150.3.1 | Peroxidase (AHRD V3.3 *** K4BHZ1_SOLLC) | 2.697038724 | 1.887755102 | UP | 6 DPA | Stress responses |
| Solyc05g006850 | Solyc05g006850.3.1 | Thioredoxin family protein (AHRD V3.3 *** | 1.792703151 | 2.675742574 | UP | 6 DPA | Stress responses |
| | | B9IJS4_POPTR) | | | | | |
| Solyc12g011310 | Solyc12g011310.2.1 | Glutathione S-transferase (AHRD V3.3 *** | 6.224431818 | 6.488647581 | UP | 20 DPA | Stress responses |
| | | Q0PN10_9FABA) | | | | | |
| Solyc03g113930 | Solyc03g113930.2.1 | Heat-shock protein, putative (AHRD V3.3 *** | 4.816214359 | 16.7543021 | UP | 20 DPA | Stress responses |
| | | B9S5K5_RICCO) | | | | | |
| Solyc06g076560 | Solyc06g076560.2.1 | 17.6 kD class I small heat shock protein | 1.792508835 | 3.91039318 | UP | 20 DPA | Stress responses |
| Solyc03g117630 | Solyc03g117630.1.1 | heat shock protein 70 (AHRD V3.3 *** | 3.838081297 | 5.919763807 | UP | 20 DPA | Stress responses |
| | | AT3G12580.1) | | | | | |
| Solyc03g082420 | Solyc03g082420.3.1 | Heat shock protein (AHRD V3.3 *** | 3.17882212 | 4.912686863 | UP | 20 DPA | Stress responses |
| | | A9QVH3_9FABA) | | | | | |
| Solyc02g093600 | Solyc02g093600.3.1 | Class I heat shock protein (AHRD V3.3 *** | 3.823698315 | 3.516725352 | UP | 20 DPA | Stress responses |
| | | F4YBC5_SOLNI) | | | | | |
| Solyc11g020330 | Solyc11g020330.1.1 | leer-sHSP small heat shock protein | 5.78839032 | 6.662272006 | UP | 20 DPA | Stress responses |
| Solyc07g056510 | Solyc07g056510.3.1 | Glutathione S-transferase (AHRD V1 **** | 10.27952756 | 20.888 | UP | 20 DPA | Stress responses |
| | | D3Y4H6_9ROSI) | | | | | |
| Solyc08g075540 | Solyc08g075540.4.1 | alternative oxidase lau | 2.849583066 | 2.64907573 | UP | 20 DPA | Stress responses |
| Solyc01g100640 | Solyc01g100640.3.1 | Catalase (AHRD V3.3 *—* LOSQ20_TOBAC) | 2.874439462 | 2.082521118 | UP | 20 DPA | Stress responses |
| Solyc01g100630 | Solyc01g100630.2.1 | Catalase (AHRD V3.3 *—* CATA_SOLAP) | 2.571348941 | 1.801249512 | UP | 20 DPA | Stress responses |
| Solyc07g054790 | Solyc07g054790.1.1 | Wound-responsive family protein (AHRD V3.3 | 0.648975965 | 0.64007338 | DOWN | 6 DPA | Stress responses |
| | | *** A0A061E3U8_THECC) | | | | | |
| Solyc04g071890 | Solyc04g071890.3.1 | Peroxidase (AHRD V3.3 *** K4BTH6_SOLLC) | 0.338122605 | 0.277297722 | DOWN | 6 DPA | Stress responses |
| Solyc07g007760 | Solyc07g007760.3.1 | defensin-like protein | 0.623769001 | 0.600975001 | DOWN | 6 DPA | Stress responses |
| Solyc07g065110 | Solyc07g065110.1.1 | Lipid transfer protein (AHRD V3.3 *** | 0.52919492 | 0.576541979 | DOWN | 20 DPA | Stress responses |
| | | G7J041_MEDTR) | | | | | |
| Solyc12g094380 | Solyc12g094380.2.1 | Thioredoxin superfamily protein (AHRD V3.3 | 0.615970842 | 0.587733165 | DOWN | 20 DPA | Stress responses |
| | | *** A0A061FEH2_THECC) | | | | | |
| Solyc01g100230 | Solyc01g100230.3.1 | DnaJ-like protein (AHRD V3.3 *** | 0.471981695 | 0.654880478 | DOWN | 20 DPA | Stress responses |
| | | D5FNB3_SOYBN) | | | | | |
| Solyc03g095180 | Solyc03g095180.3.1 | Superoxide dismutase (AHRD V3.3 *** | 0.541666667 | 0.664335664 | DOWN | 20 DPA | Stress responses |
| | | Q7YK44_SOLLC) | | | | | |
| Solyc06g005160 | Solyc06g005160.3.1 | Ascorbate peroxidase (AHRD V3.3 *** | 0.638551928 | 0.602310481 | DOWN | 20 DPA | Stress responses |
| | | Q9SMD3_SOLLC) | | | | | |
| Solyc10g083650 | Solyc10g083650.1.1 | Peroxiredoxin, putative (AHRD V3.3 *** | 0.587717723 | 0.597480832 | DOWN | 20 DPA | Stress responses |
| | | B9SRG0_RICCO) | | | | | |
| Solyc04g064690 | Solyc04g064690.3.1 | Peroxidase (AHRD V3.3 *** K4BT60_SOLLC) | 0.388 | 0.507705728 | DOWN | 20 DPA | Stress responses |
| Solyc09g011710 | Solyc09g011710.3.1 | Class I heat shock protein (AHRD V3.3 *** | 0.596822282 | 0.658960597 | DOWN | 20 DPA | Stress responses |
| | | F4YBC5_SOLNI) | | | | | |
| Solyc10g086680 | Solyc10g086680.1.1 | Class I heat shock protein (AHRD V3.3 *** | 0.615196318 | 0.557794744 | DOWN | 20 DPA | Stress responses |
| | | F4YBC5_SOLNI) | | | | | |
| Solyc01g109390 | Solyc01g109390.3.1 | Protease inhibitor/seed storage/lipid transfer | 0.321157617 | 0.455507194 | DOWN | 20 DPA | Stress responses |
| | | protein family protein (AHRD V3.3 *** | | | | | |
| | | D7MM18_ARALL) | | | | | |
| Solyc12g044420 | Solyc12g044420.1.1 | LOW QUALITY: Avr9/Cf-9 rapidly elicited | 0.464480037 | 0.488239085 | DOWN | 20 DPA | Stress responses |
| | | protein (AHRD V3.3 *** G7LFZO_MEDTR) | | | | | |
| Solyc08g005960 | Solyc08g005960.2.1 | Bifunctional inhibitor/lipid-transfer protein/seed | 0.569076869 | 0.578581115 | DOWN | 20 DPA | Stress responses |
| | | storage 2S albumin superfamily protein (AHRD | | | | | |
| | | V3.3 *** AT2G45180.1) | | | | | |
| Solyc01g099180 | Solyc01g099180.3.1 | Lipoxygenase (AHRD V3.3 *** | 0.617602428 | 0.471428571 | DOWN | 20 DPA | Stress responses |
| | | Q43800_TOBAC) | | | | | |
| Solyc07g007750 | Solyc07g007750.3.1 | Defensin protein (AHRD V3.3 *** | 0.379452621 | 0.444513741 | DOWN | 20 DPA | Stress responses |
| | | B1N678_SOLLC) | | | | | |
| Solyc07g007760 | Solyc07g007760.3.1 | defensin-like protein | 0.435407221 | 0.592067147 | DOWN | 20 DPA | Stress responses |
| Solyc05g021580 | Solyc05g021580.3.1 | Serine/threonine-protein kinase TOR (AHRD | 2.922606925 | 3.28375286 | UP | 20 DPA | TOR signaling |
| | | V3.3 *—* TOR_ARATH) | | | | | |
| Solyc10g076260 | Solyc10g076260.2.1 | Regulatory-associated protein of TOR protein | 3.815831987 | 2.197209302 | UP | 20 DPA | TOR signaling |
| | | (AHRD V3.3 *—* G7KW32_MEDTR) | | | | | |
| Solyc10g076270 | Solyc10g076270.2.1 | WD repeat-containing protein mip1 | 3.570068747 | 1.851110502 | UP | 20 DPA | TOR signaling |
| Solyc01g106770 | Solyc01g106770.3.1 | Scrinc/threonine-protein kinase (AHRD V3.3 | 1.659192825 | 1.522633745 | UP | 20 DPA | TOR signaling |
| | | *** A0A0K0XR78_TOBAC) | | | | | |
| Solyc10g050970 | Solyc10g050970.1.1 | Ethylene responsive transcription factor 2b | 1.966744874 | 1.755600073 | UP | 6 DPA | Transcription factor |
| Solyc02g077370 | Solyc02g077370.1.1 | Ethylene Response Factor C.5 | 2.189150264 | 1.981747066 | UP | 6 DPA | Transcription factor |
| Solyc08g007820 | Solyc08g007820.1.1 | C-repeat binding factor (AHRD V3.3 *** | 1.871333843 | 1.769138035 | UP | 6 DPA | Transcription factor |
| | | A0A0B5KMN4_ACTCH) | | | | | |
| Solyc03g124110 | Solyc03g124110.2.1 | CRT binding factor 2 | 2.334019293 | 1.505975104 | UP | 6 DPA | Transcription factor |
| Solyc06g035620 | Solyc06g035620.3.1 | GRAS family transcription factor (AHRD V3.3 | 1.593004769 | 1.906755471 | UP | 6 DPA | transcription factor |
| | | *** D4QD66_DIACA) | | | | | |
| Solyc10g011910 | Solyc10g011910.3.1 | WRKY transcription factor 25 | 1.744964029 | 2.636413043 | UP | 6 DPA | Transcription factor |
| Solyc08g007830 | Solyc08g007830.1.1 | C-repeat binding factor (AHRD V3.3 *** | 2.02105615 | 2.027833669 | UP | 6 DPA | Transcription factor |
| | | E7D090_PRUPE) | | | | | |
| Solyc07g022920 | Solyc07g022920.3.1 | BSD domain-containing family protein (AHRD | 5.666666667 | 12.12403101 | UP | 6 DPA | Transcription factor |
| | | V3.3 *** B9IBP6_POPTR) | | | | | |
| Solyc05g056040 | Solyc05g056040.3.1 | Auxin Response Factor 24 | 1.585884048 | 1.70763862 | UP | 20 DPA | Transcription factor |
| Solyc07g022920 | Solyc07g022920.3.1 | BSD domain-containing family protein (AHRD) | 17.08268734 | 2.748856549 | UP | 20 DPA | Transcription factor |
| | | V3.3 *** B9IBP6_POPTR) | | | | | |
| Solyc12g010310 | Solyc12g010310.2.1 | Zinc finger CCCH domain-containing protein 32 | 3.426413977 | 2.177865613 | UP | 20 DPA | Transcription factor |
| | | (AHRD V3.3 ——* C3H32_ORYSJ) | | | | | |
| Solyc02g091030 | Solyc02g091030.3.1 | Nuclear transcription factor Y subunit C-2 | 2.217877095 | 2.422701383 | UP | 20 DPA | Transcription factor |
| | | (AHRD V3.3 *—* W9SQ52_9ROSA) | | | | | |
| Solyc03g058160 | Solyc03g058160.3.1 | Zinc finger family protein (AHRD V3.3 *** | 3.284789644 | 2.362662942 | UP | 20 DPA | Transcription factor |
| | | B9HA00_POPTR) | | | | | |
| Solyc01g100660 | Solyc01g100660.3.1 | Basic helix-loop-helix transcription factor | 2.083323808 | 2.258612144 | UP | 20 DPA | Transcription factor |
| | | (AHRD V3.3 *** A0A0S3TVX2_CITUN) | | | | | |
| Solyc07g052720 | Solyc07g052720.3.1 | MADS-box transcription factor (AHRD V3.3 | 2.304771178 | 1.506300115 | UP | 20 DPA | Transcription factor |
| | | *** A0A072UYG7_MEDTR) | | | | | |
| Solyc09g074730 | Solyc09g074730.2.1 | Homeobox protein LUMINIDEPENDENS | 1.914443422 | 1.682295877 | UP | 20 DPA | Transcription factor |
| | | (AHRD V3.3 *** A0A0B2S9W0_GLYSO) | | | | | |
| Solyc05g007880 | Solyc05g007880.3.1 | Dof zinc finger protein (AHRD V3.3 *** | 1.928070823 | 1.591171994 | UP | 20 DPA | Transcription factor |
| | | W9S8B0_9ROSA) | | | | | |
| Solyc08g021820 | Solyc08g021820.3.1 | auxin-regulated IAA29 | 2.03106394 | 1.946898263 | UP | 20 DPA | Transcription factor |
| Solyc12g038450 | Solyc12g038450.1.1 | Ethylene-responsive transcription factor, putative | 6.959086584 | 5.478651685 | UP | 20 DPA | Transcription factor |
| | | (AHRD V3.3 *—* B9RVV2_RICCO) | | | | | |
| Solyc06g033850 | Solyc06g033850.3.1 | Dehydration responsive element-binding factor | 3.880482456 | 2.323703217 | UP | 20 DPA | Transcription factor |
| | | protein (AHRD V3.3 ——* | | | | | |
| | | A0A0K0K9Y3_ELECO) | | | | | |
| Solyc02g072000 | Solyc02g072000.3.1 | SolycHsfA4c | 1.630136986 | 1.621253406 | UP | 20 DPA | Transcription factor |
| Solyc08g083130 | Solyc08g083130.3.1 | Homeobox-leucine zipper protein (AHRD V3.3 | 4.826612903 | 2.164556962 | UP | 20 DPA | Transcription factor |
| | | *** K4MNF8_MEDSA) | | | | | |
| Solyc11g072500 | Solyc11g072500.2.1 | Dof domain, zinc finger family protein (AHRD | 0.580781524 | 0.492008036 | DOWN | 6 DPA | Transcription factor |
| | | V3.3 *** Q6L3K2_SOLDE) | | | | | |
| Solyc12g087830 | Solyc12g087830.2.1 | MADS-box transcription factor (AHRD V3.3 | 0.367420272 | 0.528082634 | DOWN | 6 DPA | Transcription factor |
| | | *** F1T119_9ERIC) | | | | | |
| Solyc06g035940 | Solyc06g035940.3.1 | Homeobox leucine zipper protein (AHRD V3.3 | 0.481864683 | 0.264957811 | DOWN | 6 DPA | Transcription factor |
| | | *** A0A072TNH2_MEDTR) | | | | | |
| Solyc06g066020 | Solyc06g066020.3.1 | auxin-regulated IAA36 | 0.535213591 | 0.593887262 | DOWN | 6 DPA | Transcription factor |
| Solyc03g113270 | Solyc03g113270.3.1 | LEVAHOX1G L.esculentum homeobox | 0.639419908 | 0.655848546 | DOWN | 6 DPA | Transcription factor |
| Solyc02g061990 | Solyc02g061990.3.1 | Bzip transcription factor (AHRD V3.3 *** | 0.550367314 | 0.606050532 | DOWN | 6 DPA | Transcription factor |
| | | A0A087GK23_ARAAL) | | | | | |
| Solyc12g087810 | Solyc12g087810.2.1 | MADS-box transcription factor (AHRD V3.3 | 0.372759857 | 0.664047151 | DOWN | 6 DPA | Transcription factor |
| | | *** FIT119_9ERIC) | | | | | |
| Solyc03g095770 | Solyc03g095770.3.1 | WRKY transcription factor 80 | 0.359659781 | 0.590074494 | DOWN | 6 DPA | Transcription factor |
| Solyc09g015770 | Solyc09g015770.3.1 | WRKY transcription factor 81 | 0.51974474 | 0.498273669 | DOWN | 6 DPA | Transcription factor |
| Solyc03g093610 | Solyc03g093610.1.1 | ethylene response factor A.2 | 0.555830428 | 0.621448328 | DOWN | 20 DPA | Transcription factor |
| Solyc08g077110 | Solyc08g077110.3.1 | NAC domain protein, (AHRD V3.3 *** | 0.322394678 | 0.446834665 | DOWN | 20 DPA | Transcription factor |
| | | A0A061GAH7_THECC) | | | | | |
| Solyc06g035940 | Solyc06g035940.3.1 | Homeobox leucine zipper protein (AHRD V3.3 | 0.443478261 | 0.245712886 | DOWN | 20 DPA | Transcription factor |
| | | *** A0A072TNH2_MEDTR) | | | | | |
| Solyc09g014250 | Solyc09g014250.3.1 | MYB transcription factor (AHRD V3.3 *** | 0.582278481 | 0.603410582 | DOWN | 20 DPA | Transcription factor |
| | | Q66RN1_HEVBR) | | | | | |
| Solyc11g012930 | Solyc11g012930.2.1 | WAT1-related protein (AHRD V3.3 *** | 0.593751398 | 0.636636133 | DOWN | 20 DPA | Transport |
| | | K4D697_SOLLC) | | | | | |
| Solyc04g080940 | Solyc04g080940.3.1 | WAT1-related protein (AHRD V3.3 *** | 0.55545438 | 0.532069547 | DOWN | 20 DPA | Transport |
| | | M0ZWN9_SOLTU) | | | | | |
| Solyc05g056470 | Solyc05g056470.1.1 | ABC transporter family protein (AHRD V3.3 | 0.413522013 | 0.322106552 | DOWN | 20 DPA | Transport |
| | | *** B9H498_POPTR) | | | | | |
| Solyc04g006960 | Solyc04g006960.3.1 | ABC transporter family protein (AHRD V3.3 | 0.487366167 | 0.456661316 | DOWN | 20 DPA | Transport |
| | | *** A0A097P9T3_HEVBR) | | | | | |
| Solyc07g025490 | Solyc07g025490.2.1 | OSBP(oxysterol binding protein)-related protein | 2.456157635 | 1.650993377 | UP | 20 DPA | Transport/ammonium |
| | | 4B (AHRD V3.3 ——* AT4G25850.3) | | | | | |
| Solyc03g006260 | Solyc03g006260.3.1 | Calcium-binding EF-hand (AHRD V3.3 *—* | 0.39636273 | 0.596550431 | DOWN | 20 DPA | Transport/Calcium |
| | | A0A103XMB0_CYNCS) | | | | | |
| Solyc08g075430 | Solyc08g075430.3.1 | ABC transporter family protein (AHRD V3.3 | 0.475724501 | 0.599478302 | DOWN | 20 DPA | Transport/Cytokinins |
| | | *** U5GKR9_POPTR) | | | | | |
| Solyc12g006360 | Solyc12g006360.2.1 | Multidrug resistance protein mdtK | 1.579957356 | 1.684090909 | UP | 6 DPA | Transport/Flavonoids |
| Solyc03g059150 | Solyc03g059150.3.1 | Ion channel DMI1 (AHRD V3.3 *** | 1.844827586 | 1.715166461 | UP | 20 DPA | Transport/Ion |
| | | DMI1_MEDTR) | | | | | |
| Solyc05g008780 | Solyc05g008780.3.1 | PROTON PUMP INTERACTOR 1 family | 1.875879718 | 1.542346134 | UP | 20 DPA | Transport/ion |
| | | protein (AHRD V3.3 *—* U5GXF2_POPTR) | | | | | |
| Solyc05g006640 | Solyc05g006640.3.1 | Phospholipid-transporting ATPase (AHRD V3.3 | 1.910931174 | 1.593056895 | UP | 20 DPA | Transport/Lipid |
| | | *** A0A0V0IYV6_SOLCH) | | | | | |
| Solyc01g104780 | Solyc01g104780.3.1 | Vacuolar iron transporter family protein (AHRD | 0.593037131 | 0.452713926 | DOWN | 6 DPA | Transport/Metal ion |
| | | V3.3 *** A0A061DXK2_THECC) | | | | | |
| Solyc11g012690 | Solyc11g012690.2.1 | Heavy metal transport/detoxification superfamily | 0.179023508 | 0.22147651 | DOWN | 20 DPA | Transport/Metal ion |
| | | protein (AHRD V3.3 *** 13STQ6_MEDTR) | | | | | |
| Solyc11g007200 | Solyc11g007200.2.1 | Heavy metal transport/detoxification superfamily | 0.591736695 | 0.560344828 | DOWN | 20 DPA | Transport/Metal ion |
| | | protein (AHRD V3.3 *—* AT5G27690.1) | | | | | |
| Solyc12g042240 | Solyc12g042240.2.1 | Peptide transporter (AHRD V3.3 *—* | 4.027504912 | 2.867132867 | UP | 20 DPA | Transport/Peptide |
| | | W9SG60_9ROSA) | | | | | |
| Solyc09g011390 | Solyc09g011390.3.1 | Major facilitator superfamily protein (AHRD | 0.448669202 | 0.465789474 | DOWN | 20 DPA | Transport/Peptide |
| | | V3.3 *** AT3G53960.1) | | | | | |
| Solyc01g099370 | Solyc01g099370.3.1 | SRC2 homolog | 1.51145372 | 1.615402594 | UP | 6 DPA | Transport/protein |
| Solyc03g078570 | Solyc03g078570.3.1 | Ras-related protein (AHRD V3.3 *** | 1.988673699 | 1.537911206 | UP | 20 DPA | Transport/protein |
| | | W9QNC0_9ROSA) | | | | | |
| Solyc03g058470 | Solyc03g058470.2.1 | Ypt/Rab-GAP domain of gyp1p superfamily | 2.163239075 | 1.966121495 | UP | 20 DPA | Transport/protein |
| | | protein (AHRD V3.3 *** AT2G43490.8) | | | | | |
| Solyc08g061880 | Solyc08g061880.2.1 | Peroxisome biogenesis factor 10 (AHRD V3.3 *—* | 2.232269504 | 1.8885 | UP | 20 DPA | Transport/protein |
| | | B9U2H6_TOBAC) | | | | | |
| Solyc08g048290 | Solyc08g048290.3.1 | inositol transporter 2 | 1.656700761 | 1.573349938 | UP | 20 DPA | Transport/Sugar |
| Solyc03g097590 | Solyc03g097590.3.1 | Bidirectional sugar transporter SWEET (AHRD | 2.099173554 | 7.055555556 | UP | 20 DPA | Transport/Sugar |
| | | V3.3 *** K4BJH6_SOLLC) | | | | | |
| Solyc03g097870 | Solyc03g097870.3.1 | Bidirectional sugar transporter SWEET (AHRD | 3.539928129 | 10.30901163 | UP | 20 DPA | Transport/Sugar |
| | | V3.3 *** K4BJK4_SOLLC) | | | | | |
| Solyc04g064630 | Solyc04g064630.3.1 | Bidirectional sugar transporter SWEET (AHRD | 0.501702259 | 0.439652834 | DOWN | 20 DPA | Transport/Sugar |
| | | V3.3 *** K4BT54_SOLLC) | | | | | |
| Solyc10g055630 | Solyc10g055630.2.1 | plasma membrane intrinsic protein 2.9 | 0.649546669 | 0.548698424 | DOWN | 20 DPA | Transport/Water |
| Solyc08g066840 | Solyc08g066840.3.1 | tonoplast intrinsic protein 4.1 | 0.471994965 | 0.328371278 | DOWN | 20 DPA | Transport/Water |

TABLE 6
Changes in Primary Metabolites Identified by GC-MS in adp Fruit: Placenta 6 dpa & 20 dpa

| | | PLACENTA | | | | | | | | | | | |
|---|---|---|---|---|---|---|---|---|---|---|---|---|---|
| | | 6 dpa | | | | | | 20 dpa | | | | | |
| | | adp vs. WT | | | adp vs. CL | | | adp vs. WT | | | adp vs. CL | | |
| | | FC | log2(FC) | p.ajusted | FC | log2(FC) | p.ajusted | FC | log2(FC) | p.ajusted | FC | log2(FC) | p.ajusted |
| Amino | Alanine | 0.8736 | −0.19496 | 0.055147 | 0.93446 | −0.0978 | 0.21886 | 0.87959 | −0.1851 | 0.32369 | 1.0039 | 0.005654 | 0.96263 |
| acids | Asparagine | 0.62015 | −0.68931 | 0.000403 | 0.66936 | −0.57914 | 0.003767 | 0.33021 | −1.5985 | 0.004036 | 0.36134 | −1.4686 | 0.010327 |
| | Aspartic acid | 0.86032 | −0.21705 | 0.055147 | 0.97474 | −0.03691 | 0.87352 | 0.33478 | −1.5787 | 0.000284 | 0.40989 | −1.2867 | 0.002323 |
| | beta-alanine | 0.99946 | −0.00078 | 0.96763 | 0.95974 | −0.05929 | 0.71228 | 0.82744 | −0.27328 | 0.003041 | 0.82644 | −0.27502 | 0.011707 |
| | GABA | 1.0255 | 0.036346 | 0.20269 | 1.0318 | 0.045112 | 0.36247 | 0.82976 | −0.26924 | 0.099436 | 0.87432 | −0.19376 | 0.23459 |
| | Glutamic acid | 0.69701 | −0.52074 | 0.008796 | 0.69719 | −0.52037 | 0.026099 | 0.20802 | −2.2652 | 6.11E−05 | 0.20523 | −2.2847 | 4.78E−05 |
| | Glutamine | 0.58907 | −0.76348 | 0.003178 | 0.59595 | −0.74675 | 0.027644 | 0.15388 | −2.7001 | 2.83E−06 | 0.18211 | −2.4571 | 0.000193 |
| | Isoleucine | 0.9306 | −0.10376 | 0.60686 | 0.98826 | −0.01704 | 0.89163 | 0.83846 | −0.25418 | 0.034851 | 0.97734 | −0.03306 | 0.92432 |
| | Leucine | 0.9908 | −0.01333 | 0.96763 | 0.98145 | −0.02702 | 0.88468 | 0.79259 | −0.33536 | 0.059331 | 0.93028 | −0.10426 | 0.85702 |
| | Lysine | 0.62856 | −0.66987 | 0.000403 | 0.72045 | −0.47303 | 0.003767 | 0.90257 | −0.14789 | 0.42126 | 0.93054 | −0.10386 | 0.60475 |
| | Methionine | 0.76582 | −0.38492 | 0.028868 | 0.76825 | −0.38034 | 0.027644 | 0.60789 | −0.71813 | 0.034851 | 0.68688 | −0.54187 | 0.075446 |
| | O-acetyl-serine | 0.9345 | −0.09774 | 0.59178 | 0.97273 | −0.03989 | 0.84967 | 0.94551 | −0.08083 | 0.42102 | 0.94551 | −0.08083 | 0.15884 |
| | Ornithine | 0.66157 | −0.59603 | 0.002906 | 0.6579 | −0.60406 | 0.002583 | 0.27058 | −1.8859 | 0.000811 | 0.33964 | −1.5579 | 0.00479 |
| | Phenylalanine | 0.85298 | −0.22941 | 0.055124 | 0.86646 | −0.20679 | 0.14701 | 0.69647 | −0.52186 | 0.003203 | 0.70691 | −0.5004 | 0.002892 |
| | Proline | 0.44571 | −1.1658 | 0.000403 | 0.46089 | −1.1175 | 0.001465 | 0.32435 | −1.6244 | 0.000509 | 0.34488 | −1.5358 | 0.00227 |
| | Serine | 0.86549 | −0.20841 | 0.26673 | 0.98034 | −0.02865 | 0.88468 | 0.7253 | −0.46336 | 0.13603 | 0.7777 | −0.36272 | 0.13326 |
| | Threonine | 0.58286 | −0.77879 | 0.000403 | 0.59523 | −0.74849 | 0.00351 | 0.39082 | −1.3554 | 0.000605 | 0.48717 | −1.0375 | 0.005609 |
| | Tryptophan | 1.0388 | 0.054886 | 0.88032 | 1.0426 | 0.060161 | 0.84967 | 0.90574 | −0.14283 | 0.44005 | 0.89371 | −0.16213 | 0.4735 |
| | Tyrosine | 1.0226 | 0.032304 | 0.96763 | 0.98406 | −0.02318 | 0.94113 | 0.9583 | −0.06146 | 0.73534 | 1.0912 | 0.12586 | 0.66965 |
| | Valine | 0.93708 | −0.09376 | 0.59178 | 1.0434 | 0.061326 | 0.64815 | 0.9327 | −0.10051 | 0.44005 | 1.0369 | 0.052318 | 0.7556 |
| Organic | Citric acid | 1.1511 | 0.20299 | 0.076996 | 1.0506 | 0.07128 | 0.67333 | 0.6628 | −0.59335 | 0.005534 | 0.74378 | −0.42705 | 0.025566 |
| acids | Malic acid | 1.0574 | 0.080479 | 0.59178 | 1.0137 | 0.019623 | 0.88468 | 0.96304 | −0.05433 | 0.70774 | 0.99564 | −0.0063 | 0.96263 |
| | Quinic acid-3- | 1.1358 | 0.18374 | 0.001001 | 1.1246 | 0.16947 | 0.004851 | 1.2352 | 0.30469 | 0.028625 | 1.052 | 0.0731 | 0.60475 |
| | caffeoyl | | | | | | | | | | | | |
| | Galactonic acid- | 1.1581 | 0.21176 | 0.22566 | 1.0795 | 0.11039 | 0.36247 | 0.90989 | −0.13623 | 0.28892 | 1.0552 | 0.077575 | 0.66965 |
| | 1-4-lactone | | | | | | | | | | | | |
| Main sugars | Glucose | 1.0735 | 0.10237 | 0.028868 | 1.0755 | 0.10497 | 0.038585 | 1.3993 | 0.48474 | 0.000103 | 1.3911 | 0.47619 | 0.000468 |
| | Fructose | 1.0339 | 0.048098 | 0.67553 | 1.0346 | 0.049017 | 0.75482 | 1.2157 | 0.28181 | 0.002467 | 1.1844 | 0.24412 | 0.004188 |
| | Sucrose | 0.99308 | −0.01003 | 0.88032 | 0.96585 | −0.05013 | 0.027102 | 1.2062 | 0.27042 | 0.1252 | 1.1545 | 0.20724 | 0.26396 |
| Main sugar | Glucose-6- | 2.9448 | 1.5582 | 1.40E−05 | 2.7106 | 1.4386 | 4.06E−05 | 2.4488 | 1.2921 | 0.00078 | 2.7714 | 1.4706 | 0.000468 |
| phosphates | phosphate | | | | | | | | | | | | |
| | Fructose-6- | 1.1398 | 0.18879 | 0.028868 | 1.1278 | 0.17349 | 0.21886 | 1.1303 | 0.17666 | 0.6169 | 1.1257 | 0.17088 | 0.66965 |
| | phosphate | | | | | | | | | | | | |
| Sugar alcohol | Galactinol | 1.0438 | 0.061802 | 0.88032 | 1.0432 | 0.061004 | 0.84967 | 1.0338 | 0.047945 | 0.84468 | 0.91647 | −0.12584 | 0.60475 |
| | myo-Inositol | 0.99683 | −0.00458 | 0.96763 | 0.97955 | −0.02981 | 0.71398 | 0.82298 | −0.28106 | 0.016252 | 0.87221 | −0.19725 | 0.13326 |
| | myo-Inositol-1- | 0.97411 | −0.03784 | 0.88032 | 0.93937 | −0.09023 | 0.82361 | 0.71429 | −0.48543 | 0.002308 | 0.75873 | −0.39835 | 0.005609 |
| | phosphate | | | | | | | | | | | | |
| Cell wall- | Fucose | 1.1486 | 0.19994 | 0.24661 | 1.0944 | 0.13017 | 0.42581 | 0.96306 | −0.0543 | 0.84468 | 1.0196 | 0.027937 | 0.96263 |
| related sugars | Galacturonic | 0.82059 | −0.28526 | 0.076996 | 0.80574 | −0.31161 | 0.054807 | 0.83385 | −0.26215 | 0.24616 | 0.86006 | −0.21749 | 0.26572 |
| | acid | | | | | | | | | | | | |
| | Rhamnose | 1.146 | 0.19659 | 0.20728 | 1.0778 | 0.10807 | 0.62669 | 1.0044 | 0.0063 | 0.91443 | 1.0166 | 0.023768 | 0.85702 |
| | Xylose | 1.0008 | 0.001157 | 0.96763 | 1.0175 | 0.025081 | 0.61943 | 0.99677 | −0.00467 | 0.90718 | 0.99277 | −0.01048 | 0.9 |
| Miscellaneous | Putrescine | 0.975 | −0.03653 | 0.88032 | 0.89116 | −0.16624 | 0.62089 | 1.0093 | 0.013348 | 0.86517 | 0.96805 | −0.04684 | 0.60855 |
| | Phosphoric acid | 1.0265 | 0.037772 | 0.88032 | 1.0483 | 0.068097 | 0.75482 | 0.54977 | −0.86309 | 0.002053 | 0.55197 | −0.85733 | 0.0025 |

TABLE 6
Changes in Primary Metabolites Identified by GC-MS in adp Fruit: Placenta MG & RR

| PLACENTA | |
|---|---|
| MG | RR |

| | | adp vs. WT | | | adp vs. CL | | | adp vs. WT | | | adp vs. CL | | |
|---|---|---|---|---|---|---|---|---|---|---|---|---|---|
| | | FC | log2(FC) | p.ajusted | FC | log2(FC) | p.ajusted | FC | log2(FC) | p.ajusted | FC | log2(FC) | p.ajusted |
| Amino acids | Alanine | 0.90769 | −0.13972 | 0.6048 | 1.0243 | 0.034646 | 0.8897 | 0.95903 | −0.06036 | 0.75585 | 0.99734 | −0.00385 | 0.94284 |
| | Asparagine | 0.83628 | −0.25794 | 0.16447 | 0.74118 | −0.43211 | 0.083231 | 0.56276 | −0.82942 | 0.03484 | 0.65274 | −0.61541 | 0.15627 |
| | Aspartic acid | 0.59673 | −0.74486 | 0.00772 | 0.6148 | −0.70181 | 0.003057 | 0.84371 | −0.24518 | 0.086838 | 0.89092 | −0.16663 | 0.38608 |
| | beta-alanine | 1.058 | 0.081308 | 0.65408 | 0.96813 | −0.04672 | 0.8122 | 1.1158 | 0.15806 | 0.18731 | 1.1292 | 0.17529 | 0.38266 |
| | GABA | 0.87348 | −0.19516 | 0.15424 | 0.91477 | −0.12852 | 0.25425 | 0.71137 | −0.49134 | 0.022054 | 0.74117 | −0.43212 | 0.05569 |
| | Glutamic acid | 0.33877 | −1.5616 | 1.70E−05 | 0.33466 | −1.5792 | 8.87E−06 | 0.86083 | −0.2162 | 0.15312 | 0.86595 | −0.20764 | 0.32674 |
| | Glutamine | 0.85354 | −0.22847 | 0.46437 | 0.93027 | −0.10428 | 0.60157 | 0.84217 | −0.24782 | 0.024061 | 1.0336 | 0.047682 | 0.90958 |
| | Isoleucine | 1.03 | 0.042704 | 0.86819 | 1.1892 | 0.25003 | 0.37915 | 0.88891 | −0.1699 | 0.52783 | 1.0003 | 0.000478 | 0.94868 |
| | Leucine | 0.97765 | −0.03261 | 0.86819 | 0.99742 | −0.00373 | 0.97159 | 0.86689 | −0.20609 | 0.363 | 1.0502 | 0.070695 | 0.90958 |
| | Lysine | 0.88323 | −0.17914 | 0.36583 | 0.99483 | −0.00748 | 0.97159 | 1.0119 | 0.017061 | 0.93281 | 0.97272 | −0.0399 | 0.92344 |
| | Methionine | 0.98787 | −0.01761 | 0.98055 | 1.0215 | 0.030697 | 0.85319 | 0.90012 | −0.1518 | 0.38604 | 0.94003 | −0.08923 | 0.90958 |
| | O-acetyl-serine | 0.83372 | −0.26236 | 0.028302 | 0.80543 | −0.31217 | 0.008479 | 0.91228 | −0.13245 | 0.15312 | 0.91228 | −0.13245 | 0.40684 |
| | Ornithine | 1.071 | 0.098942 | 0.46973 | 1.0547 | 0.076848 | 0.60157 | 1.0511 | 0.071893 | 0.75237 | 1.0141 | 0.020178 | 0.93049 |
| | Phenylalanine | 0.72651 | −0.46095 | 0.009977 | 0.75294 | −0.4094 | 0.005878 | 1.0474 | 0.066815 | 0.46732 | 1.1123 | 0.15354 | 0.19584 |
| | Proline | 0.80199 | −0.31834 | 0.043857 | 0.69973 | −0.51512 | 0.030007 | 0.23118 | −2.1129 | 0.009657 | 0.28558 | −1.808 | 0.022253 |
| | Serine | 0.78484 | −0.34953 | 0.095456 | 0.76775 | −0.38129 | 0.022544 | 0.94271 | −0.08512 | 0.46732 | 0.91424 | −0.12936 | 0.49785 |
| | Threonine | 1.028 | 0.039815 | 0.86819 | 0.85644 | −0.22357 | 0.44307 | 0.7564 | −0.40278 | 0.076268 | 0.86965 | −0.2015 | 0.15627 |
| | Tryptophan | 0.89415 | −0.16141 | 0.43844 | 0.85676 | −0.22303 | 0.1976 | 1.0419 | 0.05922 | 0.72649 | 1.0384 | 0.054327 | 0.92344 |
| | Tyrosine | 1.1046 | 0.14357 | 0.46437 | 0.91235 | −0.13234 | 0.37915 | 0.62933 | −0.66811 | 0.022054 | 0.61667 | −0.69742 | 0.026854 |
| | Valine | 1.0292 | 0.041577 | 0.8367 | 1.0711 | 0.099048 | 0.41787 | 0.84003 | −0.25149 | 0.15312 | 0.94696 | −0.07863 | 0.90958 |
| Organic acids | Citric acid | 0.79274 | −0.33508 | 0.36583 | 0.86599 | −0.20757 | 0.46156 | 1.0311 | 0.044141 | 0.75237 | 0.97108 | −0.04234 | 0.88921 |
| | Malic acid | 0.84482 | −0.24329 | 0.46437 | 0.89099 | −0.16653 | 0.44307 | 0.89703 | −0.15677 | 0.39334 | 0.96478 | −0.05173 | 0.93049 |
| | Quinic acid-3- | 1.0139 | 0.019916 | 0.86819 | 0.83277 | −0.264 | 0.032192 | 1.3073 | 0.38663 | 0.022288 | 1.3349 | 0.41673 | 0.05952 |
| | caffeoyl | | | | | | | | | | | | |
| | Galactonic | 1.1409 | 0.19017 | 0.46973 | 1.1776 | 0.23584 | 0.37915 | 0.97918 | −0.03035 | 0.86473 | 0.96581 | −0.05019 | 0.92344 |
| | acid-1-4- | | | | | | | | | | | | |
| | lactone | | | | | | | | | | | | |
| Main sugars | Glucose | 1.1396 | 0.18854 | 0.024235 | 1.1248 | 0.16964 | 0.032192 | 0.72727 | −0.45944 | 0.001504 | 0.72831 | −0.45737 | 0.000811 |
| | Fructose | 1.0944 | 0.13017 | 0.007019 | 1.0758 | 0.10545 | 0.005878 | 0.7932 | −0.33424 | 0.022288 | 0.85829 | −0.22046 | 0.23395 |
| | Sucrose | 1.0464 | 0.06543 | 0.49009 | 1.0315 | 0.044801 | 0.46156 | 0.841 | −0.24982 | 0.18157 | 0.84174 | −0.24856 | 0.36071 |
| Main sugar | Glucose-6- | 2.571 | 1.3623 | 5.33E−05 | 2.8132 | 1.4922 | 0.000603 | 2.7687 | 1.4692 | 0.001504 | 2.9269 | 1.5494 | 0.000811 |
| | phosphate | | | | | | | | | | | | |
| phosphates | Fructose-6- | 1.0229 | 0.032605 | 0.86623 | 0.94046 | −0.08857 | 0.65711 | 1.1059 | 0.14528 | 0.39563 | 0.93701 | −0.09387 | 0.90958 |
| | phosphate | | | | | | | | | | | | |
| Sugar alcohol | Galactinol | 1.0791 | 0.10989 | 0.68028 | 0.85628 | −0.22385 | 0.25425 | 1.1649 | 0.22023 | 0.39334 | 1.0251 | 0.035717 | 0.93049 |
| | myo-Inositol | 0.56728 | −0.81788 | 0.002554 | 0.55285 | −0.85504 | 0.001593 | 0.59573 | −0.74727 | 0.036138 | 0.56551 | −0.82238 | 0.05569 |
| | myo-Inositol- | 0.48246 | −1.0515 | 0.009977 | 0.51643 | −0.95335 | 0.011471 | 0.58161 | −0.78187 | 0.015768 | 0.60429 | −0.72669 | 0.11641 |
| | 1-phosphate | | | | | | | | | | | | |
| Cell wall- | Fucose | 0.71175 | −0.49055 | 0.054375 | 0.77913 | −0.36007 | 0.15155 | 0.87935 | −0.1855 | 0.15312 | 0.89027 | −0.16769 | 0.3594 |
| related sugars | Galacturonic | 1.1339 | 0.1813 | 0.50802 | 0.9861 | −0.0202 | 0.94654 | 0.75056 | −0.41395 | 0.28017 | 0.94752 | −0.07777 | 0.93049 |
| | acid | | | | | | | | | | | | |
| | Rhamnose | 1.0731 | 0.10181 | 0.52203 | 1.0796 | 0.11056 | 0.47543 | 1.1355 | 0.18336 | 0.15312 | 1.1199 | 0.16335 | 0.43318 |
| | Xylose | 1.0165 | 0.023593 | 0.52203 | 0.99838 | −0.00234 | 0.97159 | 0.99317 | −0.00989 | 0.84895 | 0.98643 | −0.01972 | 0.92344 |
| Miscellaneous | Putrescine | 0.94711 | −0.0784 | 0.76173 | 0.91649 | −0.12581 | 0.46156 | 0.94013 | −0.08906 | 0.52783 | 0.92308 | −0.11548 | 0.90958 |
| | Phosphoric | 1.0165 | 0.023649 | 0.86819 | 1.0361 | 0.051137 | 0.60157 | 0.93866 | −0.09133 | 0.51247 | 1.0244 | 0.034719 | 0.93049 |
| | acid | | | | | | | | | | | | |

Changes in Primary Metabolites Identified by GC-MS in adp Fruit: Pericarp 6 dpa & 20 dpa

| | | PERICARP | | | | | | | | | | | |
|---|---|---|---|---|---|---|---|---|---|---|---|---|---|
| | | 6 dpa | | | | | | 20 dpa | | | | | |
| | | adp vs. WT | | | adp vs. CL | | | adp vs. WT | | | adp vs. CL | | |
| | | FC | log2(FC) | p.ajusted | FC | log2(FC) | p.ajusted | FC | log2(FC) | p.ajusted | FC | log2(FC) | p.ajusted |
| Amino acids | Alanine | 0.66163 | −0.5959 | 0.16081 | 0.70136 | −0.51177 | 0.3675 | 1.3113 | 0.39095 | 0.046387 | 1.1419 | 0.1914 | 0.41079 |
| | Asparagine | 0.90816 | −0.13898 | 0.014671 | 0.96644 | −0.04925 | 0.77394 | 0.41732 | −1.2608 | 0.000206 | 0.48752 | −1.0365 | 0.000266 |
| | Aspartic acid | 0.90802 | −0.1392 | 0.06179 | 1.0421 | 0.059429 | 0.43453 | 0.60906 | −0.71534 | 5.05E−05 | 0.71871 | −0.47653 | 0.003176 |
| | beta-alanine | 1.0466 | 0.065719 | 0.47029 | 1.0349 | 0.049449 | 0.64518 | 0.75159 | −0.41198 | 0.000206 | 0.74283 | −0.42889 | 0.000266 |
| | GABA | 0.961 | −0.05739 | 0.47029 | 0.99245 | −0.01093 | 0.83765 | 0.96178 | −0.05621 | 0.16075 | 0.96775 | −0.04729 | 0.41203 |
| | Glutamic acid | 0.70827 | −0.49762 | 0.025061 | 0.77358 | −0.37038 | 0.08501 | 0.63301 | −0.65969 | 5.05E−05 | 0.67868 | −0.55919 | 0.000266 |
| | Glutamine | 0.96214 | −0.05569 | 0.65889 | 1.0121 | 0.017303 | 0.821 | 0.53656 | −0.89818 | 0.000328 | 0.59577 | −0.74718 | 0.003176 |
| | Isoleucine | 1.0825 | 0.11433 | 0.61125 | 1.1057 | 0.14497 | 0.64518 | 0.89594 | −0.15853 | 0.11888 | 0.89993 | −0.15212 | 0.54613 |
| | Leucine | 1.0442 | 0.062453 | 0.54703 | 1.0376 | 0.053232 | 0.66807 | 0.99921 | −0.00114 | 0.9771 | 1.0493 | 0.069456 | 0.54613 |
| | Lysine | 1.0337 | 0.047837 | 0.72301 | 0.97561 | −0.03562 | 0.821 | 0.90276 | −0.14758 | 0.1598 | 0.96293 | −0.0545 | 0.70212 |
| | Methionine | 1.0942 | 0.12987 | 0.21798 | 1.0905 | 0.12503 | 0.3675 | 0.93418 | −0.09822 | 0.46315 | 0.95291 | −0.06959 | 0.70212 |
| | O-acetyl-serine | 0.9557 | −0.06538 | 0.51913 | 1.0134 | 0.019236 | 0.83765 | 1.0129 | 0.018437 | 0.68052 | 1.0227 | 0.032421 | 0.55226 |
| | Ornithine | 0.94602 | −0.08006 | 0.47029 | 0.94792 | −0.07717 | 0.64518 | 1.1233 | 0.16769 | 0.30599 | 1.1637 | 0.21878 | 0.28187 |
| | Phenylalanine | 1.3015 | 0.38015 | 0.002441 | 1.3057 | 0.3848 | 0.001909 | 0.9361 | −0.09527 | 0.037266 | 0.94514 | −0.0814 | 0.33218 |
| | Proline | 0.48488 | −1.0443 | 0.010451 | 0.57879 | −0.78889 | 0.073571 | 0.39099 | −1.3548 | 0.000199 | 0.46253 | −1.1124 | 0.002199 |
| | Serine | 1.0107 | 0.015285 | 0.79318 | 0.94151 | −0.08695 | 0.43453 | 0.83647 | −0.25762 | 0.037967 | 0.90131 | −0.1499 | 0.11702 |
| | Threonine | 1.097 | 0.13355 | 0.45426 | 1.0614 | 0.085934 | 0.64518 | 0.89525 | −0.15964 | 0.16075 | 0.96272 | −0.05481 | 0.55226 |
| | Tryptophan | 1.0397 | 0.056137 | 0.70447 | 1.0814 | 0.11294 | 0.43453 | 0.92832 | −0.1073 | 0.5719 | 0.92087 | −0.11893 | 0.4689 |
| | Tyrosine | 1.0961 | 0.1324 | 0.55164 | 1.2017 | 0.26513 | 0.15013 | 1.0341 | 0.048324 | 0.71338 | 0.98854 | −0.01663 | 0.95752 |
| | Valine | 0.95895 | −0.06047 | 0.61125 | 0.93094 | −0.10323 | 0.43453 | 0.92815 | −0.10757 | 0.45483 | 0.94903 | −0.07547 | 0.54613 |
| Organic acids | Citric acid | 1.0274 | 0.038989 | 0.36184 | 1.0259 | 0.03683 | 0.3675 | 0.84438 | −0.24404 | 0.000537 | 0.82293 | −0.28115 | 0.003176 |
| | Malic acid | 0.99552 | −0.00648 | 0.79318 | 1.017 | 0.024326 | 0.64518 | 0.9583 | −0.06145 | 0.46315 | 1.019 | 0.027184 | 0.56638 |
| | Quinic acid-3- | 0.93391 | −0.09864 | 0.36184 | 1.0289 | 0.04106 | 0.64518 | 3.0463 | 1.607 | 0.60675 | 3.5046 | 1.8093 | 0.54613 |
| | caffeoyl | | | | | | | | | | | | |
| | Galactonic | 0.97898 | −0.03066 | 0.72301 | 0.92815 | −0.10757 | 0.38648 | 0.98208 | −0.02609 | 0.89242 | 0.96701 | −0.0484 | 0.64254 |
| | acid-1-4- | | | | | | | | | | | | |
| | lactone | | | | | | | | | | | | |
| Main sugars | Glucose | 1.0322 | 0.045696 | 0.60749 | 1.0314 | 0.04455 | 0.64518 | 1.0687 | 0.095829 | 0.039303 | 1.0994 | 0.13666 | 0.027772 |
| | Fructose | 1.1247 | 0.16955 | 0.47029 | 0.99135 | −0.01254 | 0.92884 | 1.013 | 0.018624 | 0.68052 | 1.0314 | 0.044642 | 0.40978 |
| | Sucrose | 1.0586 | 0.082209 | 0.67051 | 1.022 | 0.031339 | 0.83765 | 1.0333 | 0.047299 | 0.62369 | 1.0001 | 3.01E−05 | 0.98044 |
| Main sugar | Glucose-6- | 2.0342 | 1.0244 | 3.21E−05 | 2.013 | 1.0093 | 2.68E−06 | 3.0537 | 1.6106 | 5.05E−05 | 3.0921 | 1.6286 | 0.000139 |
| | phosphate | | | | | | | | | | | | |
| phosphates | Fructose-6- | 1.0211 | 0.03008 | 0.77469 | 1.0744 | 0.10357 | 0.38648 | 1.0518 | 0.072879 | 0.5719 | 0.86752 | −0.20503 | 0.29692 |
| | phosphate | | | | | | | | | | | | |
| Sugar alcohol | Galactinol | 1.0109 | 0.015676 | 0.79318 | 1.0168 | 0.024036 | 0.72041 | 0.86212 | −0.21405 | 0.070139 | 0.71275 | −0.48854 | 0.002097 |
| | myo-Inositol | 1.0282 | 0.040133 | 0.47029 | 1.0813 | 0.11274 | 0.10708 | 0.84114 | −0.24959 | 0.027867 | 0.92186 | −0.11738 | 0.46597 |
| | myo-Inositol-1- | 1.2019 | 0.26535 | 0.16081 | 1.2402 | 0.31053 | 0.11072 | 0.88901 | −0.16972 | 0.3636 | 0.97445 | −0.03734 | 0.89218 |
| | phosphate | | | | | | | | | | | | |
| Cell wall- | Fucose | 0.57953 | −0.78705 | 0.01023 | 0.63198 | −0.66206 | 0.014527 | 0.76627 | −0.38408 | 0.00924 | 0.73611 | −0.442 | 0.003176 |
| related sugars | Galacturonic | 0.83437 | −0.26124 | 0.10597 | 0.97379 | −0.03831 | 0.83765 | 0.93015 | −0.10446 | 0.48484 | 1.1362 | 0.18422 | 0.40978 |
| | acid | | | | | | | | | | | | |
| | Rhamnose | 0.99309 | −0.01001 | 0.85551 | 1.0527 | 0.074067 | 0.59222 | 0.91929 | −0.12141 | 0.5719 | 0.90889 | −0.13783 | 0.46597 |
| | Xylose | 0.99168 | −0.01206 | 0.77469 | 1.0162 | 0.023251 | 0.64518 | 1.0033 | 0.004692 | 0.90921 | 1.0216 | 0.030772 | 0.48843 |
| Miscellaneous | Putrescine | 1.0681 | 0.094985 | 0.54703 | 1.0349 | 0.049506 | 0.72459 | 0.91569 | −0.12707 | 0.38304 | 0.85705 | −0.22255 | 0.13135 |
| | Phosphoric acid | 0.9931 | −0.00999 | 0.47029 | 1.0074 | 0.010589 | 0.72041 | 0.9498 | −0.0743 | 0.3636 | 0.92565 | −0.11146 | 0.16445 |

Changes in Primary Metabolites Identified by GC-MS in adp Fruit: Pericarp MG & RR

| | | PERICARP | | | | | | | | | | | |
|---|---|---|---|---|---|---|---|---|---|---|---|---|---|
| | | MG | | | | | | Ripe | | | | | |
| | | adp vs. WT | | | adp vs. CL | | | adp vs. WT | | | adp vs. CL | | |
| | | FC | log2(FC) | p.ajusted | FC | log2(FC) | p.ajusted | FC | log2(FC) | p.ajusted | FC | log2(FC) | p.ajusted |
| Amino acids | Alanine | 0.88029 | −0.18394 | 0.64502 | 1.0552 | 0.0775 | 0.8793 | 1.0025 | 0.00361 | 0.97145 | 0.92979 | −0.10502 | 0.62298 |
| | Asparagine | 1.0006 | 0.000927 | 0.96478 | 1.0701 | 0.09775 | 0.8477 | 0.87996 | −0.18448 | 0.13428 | 0.9975 | −0.00361 | 0.96853 |
| | Aspartic acid | 0.62321 | −0.68222 | 0.001062 | 0.7088 | −0.49654 | 0.003434 | 0.66115 | −0.59695 | 0.006025 | 0.82224 | −0.28237 | 0.070038 |
| | beta-alanine | 0.91458 | −0.12882 | 0.49124 | 0.89801 | −0.15519 | 0.35804 | 0.95674 | −0.06381 | 0.4232 | 0.92636 | −0.11036 | 0.26833 |
| | GABA | 0.79623 | −0.32873 | 0.009214 | 0.77826 | −0.36168 | 0.013609 | 0.55219 | −0.85675 | 0.000121 | 0.65853 | −0.60269 | 0.026035 |
| | Glutamic acid | 0.61214 | −0.70806 | 0.001062 | 0.66727 | −0.58367 | 0.003434 | 0.93164 | −0.10216 | 0.10276 | 0.96388 | −0.05307 | 0.69155 |
| | Glutamine | 0.69762 | −0.51948 | 0.20996 | 0.79009 | −0.33992 | 0.35804 | 0.90147 | −0.14965 | 0.59693 | 0.94972 | −0.07443 | 0.79759 |
| | Isoleucine | 0.77505 | −0.36764 | 0.72668 | 0.92172 | −0.11759 | 0.8973 | 0.70736 | −0.49948 | 0.29246 | 0.78406 | −0.35096 | 0.65782 |
| | Leucine | 0.74602 | −0.4227 | 0.27204 | 0.92257 | −0.11626 | 0.95769 | 0.78465 | −0.34988 | 0.13471 | 0.8935 | −0.16246 | 0.69155 |
| | Lysine | 1.0822 | 0.11396 | 0.49124 | 1.0067 | 0.009629 | 0.96078 | 0.88727 | −0.17256 | 0.20311 | 0.90087 | −0.1506 | 0.62298 |
| | Methionine | 0.88009 | −0.18428 | 0.55942 | 0.82036 | −0.28567 | 0.43534 | 0.71148 | −0.49111 | 0.011226 | 0.76635 | −0.38392 | 0.026035 |
| | O-acetyl-serine | 0.94462 | −0.0822 | 0.72668 | 0.91642 | −0.12592 | 0.69682 | 1.0285 | 0.040515 | 0.40459 | 0.99388 | −0.00885 | 0.96853 |
| | Ornithine | 0.90803 | −0.1392 | 0.40848 | 0.9536 | −0.06855 | 0.8846 | 1.1009 | 0.13866 | 0.33303 | 1.0944 | 0.13019 | 0.4856 |
| | Phenylalanine | 0.93148 | −0.1024 | 0.49124 | 0.97242 | −0.04035 | 0.8793 | 0.85476 | −0.2264 | 0.30864 | 0.97331 | −0.03904 | 0.82946 |
| | Proline | 0.61397 | −0.70375 | 0.022754 | 0.67782 | −0.56103 | 0.099311 | 0.94293 | −0.08477 | 0.62327 | 1.0161 | 0.023022 | 0.96853 |
| | Serine | 0.87231 | −0.19708 | 0.46106 | 0.89781 | −0.15552 | 0.35804 | 0.97848 | −0.03138 | 0.90238 | 1.0334 | 0.047454 | 0.89393 |
| | Threonine | 0.90621 | −0.14209 | 0.20996 | 0.93432 | −0.09801 | 0.50215 | 0.86177 | −0.21463 | 0.33303 | 0.94067 | −0.08824 | 0.72873 |
| | Tryptophan | 1.0386 | 0.05464 | 0.92907 | 1.0823 | 0.11413 | 0.8477 | 0.51957 | −0.9446 | 0.003505 | 0.66346 | −0.59192 | 0.018466 |
| | Tyrosine | 1.0049 | 0.007053 | 0.96478 | 0.99874 | −0.00181 | 0.96078 | 0.67635 | −0.56416 | 0.018496 | 0.72708 | −0.45982 | 0.070038 |
| | Valine | 0.92953 | −0.10543 | 0.31491 | 0.84116 | −0.24955 | 0.010046 | 0.76337 | −0.38955 | 0.006025 | 0.78972 | −0.34059 | 0.26833 |
| Organic acids | Citric acid | 0.9981 | −0.00275 | 0.96478 | 1.029 | 0.041239 | 0.1416 | 1.0003 | 0.000376 | 0.97145 | 0.94128 | −0.0873 | 0.070038 |
| | Malic acid | 1.0038 | 0.005426 | 0.96478 | 1.0092 | 0.013183 | 0.96078 | 0.81111 | −0.30204 | 0.30864 | 0.94402 | −0.08311 | 0.74874 |
| | Quinic acid-3- | 0.95229 | −0.07052 | 0.87583 | 0.34416 | −1.5388 | 0.87106 | 0.94248 | −0.08547 | 0.29246 | 0.94667 | −0.07907 | 0.69155 |
| | caffeoyl | | | | | | | | | | | | |
| | Galactonic acid- | 0.96415 | −0.05267 | 0.72668 | 1.0287 | 0.040851 | 0.8477 | 0.88863 | −0.17035 | 0.12748 | 0.94985 | −0.07424 | 0.69155 |
| | 1-4-lactone | | | | | | | | | | | | |
| Main sugars | Glucose | 1.0457 | 0.06449 | 0.55942 | 1.007 | 0.010085 | 0.96078 | 0.74501 | −0.42467 | 0.000102 | 0.77084 | −0.3755 | 0.001868 |
| | Fructose | 1.0305 | 0.043374 | 0.40848 | 1.0137 | 0.019632 | 0.8477 | 0.95874 | −0.06079 | 0.15233 | 0.97507 | −0.03643 | 0.65782 |
| | Sucrose | 1.0602 | 0.084334 | 0.46106 | 1.0015 | 0.002133 | 0.96078 | 0.76642 | −0.38379 | 0.003505 | 0.78578 | −0.34781 | 0.018466 |
| Main sugar | Glucose-6- | 3.0182 | 1.5937 | 1.76E−05 | 2.9567 | 1.564 | 0.000137 | 2.4871 | 1.3145 | 0.00018 | 2.493 | 1.3179 | 0.000307 |
| | phosphate | | | | | | | | | | | | |
| phosphates | Fructose-6- | 0.9784 | −0.03151 | 0.85656 | 1.1007 | 0.13841 | 0.013609 | 0.95022 | −0.07367 | 0.87789 | 0.91649 | −0.1258 | 0.74874 |
| | phosphate | | | | | | | | | | | | |
| Sugar alcohol | Galactinol | 1.0057 | 0.008193 | 0.96478 | 0.97461 | −0.0371 | 0.8973 | 0.78878 | −0.34231 | 0.29246 | 0.82883 | −0.27085 | 0.1537 |
| | myo-Inositol | 0.77929 | −0.35976 | 0.27204 | 0.85084 | −0.23304 | 0.59048 | 0.46019 | −1.1197 | 0.000102 | 0.4132 | −1.2751 | 0.000307 |
| | myo-Inositol-1- | 0.98015 | −0.02893 | 0.96478 | 0.9947 | −0.00767 | 0.96078 | 1.001 | 0.001422 | 0.97145 | 0.92441 | −0.1134 | 0.69155 |
| | phosphate | | | | | | | | | | | | |
| Cell wall- | Fucose | 1.0824 | 0.1143 | 0.5161 | 0.96217 | −0.05563 | 0.87106 | 0.94947 | −0.07481 | 0.59693 | 0.76774 | −0.38131 | 0.024229 |
| related sugars | Galacturonic acid | 0.94977 | −0.07434 | 0.81061 | 0.94835 | −0.07652 | 0.8477 | 0.79294 | −0.33471 | 0.30864 | 1.0046 | 0.006624 | 0.96717 |
| | Rhamnose | 0.98555 | −0.021 | 0.96478 | 0.92434 | −0.11351 | 0.8477 | 0.9709 | −0.04261 | 0.88444 | 0.83333 | −0.26303 | 0.084248 |
| | Xylose | 0.99719 | −0.00406 | 0.96478 | 1.0085 | 0.012244 | 0.8973 | 0.98985 | −0.01472 | 0.83575 | 0.98109 | −0.02755 | 0.69155 |
| Miscellaneous | Putrescine | 0.86627 | −0.20712 | 0.27204 | 0.88285 | −0.17976 | 0.35804 | 0.92751 | −0.10856 | 0.20741 | 0.96591 | −0.05004 | 0.74874 |
| | Phosphoric acid | 0.96003 | −0.05884 | 0.70739 | 0.99742 | −0.00372 | 0.96078 | 0.97868 | −0.03109 | 0.55511 | 0.96012 | −0.05872 | 0.6022 |

TABLE 7
Changes in Metabolites Identified in adp Fruit Using an Untargeted LC-MS/MS Analysis

| | | | | | | In-house | | | | | | |
|---|---|---|---|---|---|---|---|---|---|---|---|---|
| | | | | | mzCloud | spectral | | | | | | |
| | | | | | Best | library | | | | adp vs. WT | | |
| | | | Molecular | RT | Match | Best | adp vs. CL | | | | | adjusted |
| Name | Mode | Formula | Weight | [min] | (%) | Match (%) | FC | log2(FC) | adjusted p | FC | log2(FC) | p |
| α-D-Mannose 1- | C18_NEG | C6 H13 O9 P | 260.02907 | 0.999 | 92.2 | | 3.8117 | 1.9304 | 0.02612 | 3.6385 | 1.8633 | 0.049427 |
| phosphate | | | | | | | | | | | | |
| N-Undecanoylglycine | HILIC_POS | C13 H25 N O3 | 243.18367 | 2.741 | | | 2.964 | 1.5675 | 0.017356 | 2.1417 | 1.0987 | 0.034121 |
| L-2-aminodecanoate | C18_POS | C10 H21 N O2 | 187.1575 | 8.462 | | | 2.6918 | 1.4286 | 0.00591 | 7.1891 | 2.8458 | 0.00053 |
| 3-Hydroxy-3- | HILIC_NEG | C6 H10 O5 | 162.05312 | 9.182 | 82.6 | | 2.5267 | 1.3372 | 0.005756 | 2.7669 | 1.4683 | 0.007556 |
| methylglutaric acid | | | | | | | | | | | | |
| (2R)-2-Hydroxy-3- | HILIC_NEG | C20 H41 O7 P | 424.25916 | 1.352 | | | 2.4494 | 1.2924 | 0.000367 | 3.1012 | 1.6328 | 0.000551 |
| (phosphonooxy)propyl | | | | | | | | | | | | |
| heptadecanoate | | | | | | | | | | | | |
| 2′-hydroxy 3,6,7,4′- | HILIC_POS | C25 H28 O14 | 552.14733 | 8.419 | | | 2.0895 | 1.0632 | 0.002747 | 2.2261 | 1.1545 | 0.001682 |
| tetramethylquercetag | | | | | | | | | | | | |
| etin 3′-O-β-D- | | | | | | | | | | | | |
| glucoside | | | | | | | | | | | | |
| cyanidin 3,5-di-O-β- | HILIC_POS | C27 H31 O16 | 610.15361 | 8.345 | | | 2.0073 | 1.0053 | 0.016372 | 1.767 | 0.82132 | 0.040258 |
| D-glucoside | | | | | | | | | | | | |
| cyanidin 3,5-di-O-β- | C18_NEG | C27 H31 O16 | 610.15274 | 9.498 | | | 1.9448 | 0.95963 | 0.009704 | 1.9837 | 0.98818 | 0.02163 |
| D-glucoside | | | | | | | | | | | | |
| D-Raffinose | HILIC_POS | C18 H32 O16 | 526.15127 | 9.169 | 88.8 | | 1.8962 | 0.92314 | 0.002822 | 1.6311 | 0.70587 | 0.001451 |
| Adenosine | HILIC_POS | C10 H13 N5 O4 | 267.09688 | 4.922 | 94.9 | | 1.8912 | 0.91927 | 0.028026 | 1.5854 | 0.6648 | 0.014428 |
| Acetylcholine | HILIC_POS | C7 H15 N O2 | 145.11049 | 5.557 | 86.4 | | 1.8591 | 0.8946 | 0.008922 | 1.9723 | 0.97988 | 0.004309 |
| 1-O-feruloyl-β-D- | C18_NEG | C16 H20 O9 | 356.11006 | 9.407 | | | 1.8386 | 0.87857 | 0.019436 | 1.9845 | 0.98876 | 0.017945 |
| glucose | | | | | | | | | | | | |
| D-(+)-Glucose | HILIC_POS | C6 H12 O6 | 197.08991 | 7.607 | 87.2 | | 1.8034 | 0.85075 | 0.001791 | 1.651 | 0.72335 | 0.001057 |
| 3-(3_4- | C18_POS | C9 H10 O5 | 198.05254 | 1.012 | | | 1.767 | 0.82133 | 0.004672 | 1.7864 | 0.83706 | 0.001007 |
| Dihydroxyphenyl)lactate | | | | | | | | | | | | |
| 4-Methoxychalcone | HILIC_POS | C16 H14 O2 | 238.09897 | 8.314 | 56.2 | | 1.7518 | 0.80884 | 0.014009 | 2.4419 | 1.288 | 5.26E−05 |
| 5-hydroxy- | C18_POS | C10 H10 O4 | 194.05822 | 9.385 | | | 1.7503 | 0.8076 | 0.009457 | 1.4257 | 0.51169 | 0.009377 |
| coniferaldehyde | | | | | | | | | | | | |
| 2-keto-L-xylose | C18_POS | C5 H8 O5 | 148.03707 | 1.024 | | | 1.7431 | 0.80161 | 0.032587 | 2.1571 | 1.1091 | 0.008134 |
| D-(+)-Mannose | HILIC_NEG | C6 H12 O6 | 180.06369 | 8.204 | 91 | | 1.7066 | 0.77112 | 0.003904 | 1.7599 | 0.81547 | 0.004162 |
| 3_4-Dihydroxymandelaldehyde | C18 POS | C8 H8 O4 | 168.04212 | 1.01 | | | 1.6891 | 0.75628 | 0.011451 | 1.8823 | 0.9125 | 0.000588 |
| Sinapinic acid | C18_POS | C11 H12 O5 | 224.0681 | 1.008 | 84.8 | | 1.6668 | 0.73704 | 0.002219 | 2.7835 | 1.4769 | 0.000733 |
| (4Z)-3- | C18_POS | C10 H12 O6 | 228.0631 | 1.004 | | | 1.6604 | 0.73149 | 0.002442 | 1.7106 | 0.77453 | 0.001905 |
| (Dihydroxymethylene)- | | | | | | | | | | | | |
| 7-methyl-2,6- | | | | | | | | | | | | |
| dioxo-4-octenoic | | | | | | | | | | | | |
| acid | | | | | | | | | | | | |
| D-Saccharic acid | C18_NEG | C6 H10 O8 | 210.03725 | 1.022 | 83.3 | 94.1 | 1.6452 | 0.71826 | 0.027487 | 1.493 | 0.57818 | 0.026458 |
| pyridoxal | C18_POS | C8 H9 N O3 | 167.058 | 1.011 | | | 1.6447 | 0.71785 | 0.007387 | 1.5278 | 0.61148 | 0.003859 |
| 2-benzyl-3- | C18_POS | C11 H12 O5 | 222.05241 | 1.005 | | | 1.6391 | 0.71287 | 0.0004 | 2.1135 | 1.0797 | 0.000242 |
| hydroxybutanedioate | | | | | | | | | | | | |
| hypotaurine | HILIC_POS | C2 H7 N O2 S | 109.01998 | 8.344 | | | 1.6218 | 0.69758 | 0.005349 | 2.3057 | 1.2052 | 0.000157 |
| 9(Z),11(E)- | HILIC_NEG | C18 H32 O2 | 280.24019 | 1.403 | | | 1.5993 | 0.4847 | 0.033221 | 1.5669 | 0.55274 | 0.03217 |
| Conjugated linoleic | | | | | | | | | | | | |
| acid | | | | | | | | | | | | |
| D-Glucose 6- | HILIC_POS | C6 H13 O9 P | 260.02996 | 9.925 | 85.6 | | 1.589 | 0.66809 | 0.000661 | 1.5192 | 0.60333 | 0.001682 |
| phosphate | | | | | | | | | | | | |
| (2R)-2-[(9Z)-9- | HILIC_POS | C38 H72 N O8 P | 701.50009 | 1.118 | | | 1.5879 | 0.66714 | 0.014976 | 1.662 | 0.7329 | 0.034361 |
| Hexadecenoyloxy]-3- | | | | | | | | | | | | |
| [(9Z)-9- | | | | | | | | | | | | |
| tetradecenoyloxy]propyl | | | | | | | | | | | | |
| 2-(trimethylammonio)ethyl | | | | | | | | | | | | |
| phosphate | | | | | | | | | | | | |
| benzyl-2-methyl-3- | C18_POS | C12 H14 O3 | 206.09452 | 9.433 | | | 1.5855 | 0.66491 | 0.039554 | 1.509 | 0.5936 | 0.013102 |
| oxobutanoate | | | | | | | | | | | | |
| 1_3_6_8- | C18_POS | C10 H8 O4 | 192.04189 | 1.011 | | | 1.5654 | 0.64649 | 0.011886 | 1.6837 | 0.75161 | 0.004598 |
| Naphthalenetetrol | | | | | | | | | | | | |
| Chlorogenic acid | HILIC_POS | C16 H18 O9 | 752.16902 | 7.488 | 78.7 | | 1.5653 | 0.64641 | 0.008434 | 2.0575 | 1.0409 | 7.00E−05 |
| Pyridoxal | HILIC_POS | C8 H9 N O3 | 167.05849 | 5.689 | 84.1 | | 1.5603 | 0.64182 | 0.001452 | 1.33 | 0.41144 | 0.006923 |
| β-D-ribosylnicotinate | HILIC_POS | C11 H14 N O6 | 255.07428 | 7.453 | | | 1.5479 | 0.63036 | 0.00125 | 1.3536 | 0.43681 | 0.007061 |
| Phenyl D- | C18 POS | C12 H14 O7 | 270.07341 | 1.007 | | | 1.5412 | 0.62408 | 0.001786 | 1.8789 | 0.90987 | 0.00056 |
| glucopyranosiduronic | | | | | | | | | | | | |
| acid | | | | | | | | | | | | |
| 2-Succinylbenzoate | C18_POS | C11 H10 O5 | 222.05225 | 1.009 | | | 1.539 | 0.62195 | 0.001555 | 1.7629 | 0.81796 | 9.66E−05 |
| L-erythrulose | HILIC_NEG | C4 H8 O4 | 120.04254 | 8.183 | | | 1.5362 | 0.61941 | 0.002404 | 1.6179 | 0.69416 | 0.001959 |
| Adenine | HILIC_NEG | C5 H5 N5 | 135.05477 | 4.894 | 51.3 | | 1.5284 | 0.61202 | 0.000586 | 1.2185 | 0.28516 | 0.018246 |
| 2-Hydroxy-3,4,5- | C18_POS | C10 H12 O6 | 228.0631 | 1.01 | | | 1.5161 | 0.60036 | 0.038041 | 1.3828 | 0.46758 | 0.040874 |
| trimethoxybenzoic | | | | | | | | | | | | |
| acid | | | | | | | | | | | | |
| 1,5-Anhydro-1-(2,4,6- | C18_POS | C12 H16 O8 | 288.084 | 1.016 | | | 1.5077 | 0.59231 | 0.007091 | 1.6217 | 0.69751 | 0.037876 |
| trihydroxyphenyl)hexitol | | | | | | | | | | | | |
| 3-Methylcrotonylglycine | HILIC_POS | C7 H11 N O3 | 157.0741 | 8.097 | 33.6 | | 1.507 | 0.59171 | 0.003581 | 2.7849 | 1.4776 | 0.000273 |
| (1R_6R)-6-Hydroxy- | C18_POS | C11 H12 O6 | 240.06298 | 1.012 | | | 1.5057 | 0.59041 | 0.011375 | 1.6785 | 0.74715 | 0.000428 |
| 2-succinylcyclohexa- | | | | | | | | | | | | |
| 2_4-diene-1- | | | | | | | | | | | | |
| carboxylate | | | | | | | | | | | | |
| 2_3-Dihydro-2_3- | C18_POS | C7 H8 O4 | 156.04216 | 1.037 | | | 1.4988 | 0.5838 | 0.039355 | 1.3507 | 0.43375 | 0.017195 |
| dihydroxybenzoate | | | | | | | | | | | | |
| DL-Lactic Acid | HILIC_NEG | C3 H6 O3 | 90.03183 | 8.182 | 59.3 | | 1.4948 | 0.57999 | 0.000463 | 1.5662 | 0.64722 | 0.001229 |
| Taurine | HILIC_NEG | C2 H7 N O3 S | 125.01489 | 8.207 | 87.9 | | 1.4899 | 0.3236 | 0.010834 | 1.7855 | 0.83633 | 0.000112 |
| Scopoletin | C18_POS | C10 H8 O4 | 192.04198 | 1.006 | | | 1.4896 | 0.57491 | 0.000526 | 2.1286 | 1.0899 | 0.000866 |
| D-Glucosamine | C18_POS | C6 H13 N O5 | 179.07912 | 1.011 | 70.1 | | 1.4765 | 0.56222 | 0.003453 | 1.6001 | 0.67814 | 0.001291 |
| Phenylglyoxylic acid | C18_POS | C8 H6 O3 | 150.03147 | 1.012 | | | 1.4715 | 0.55732 | 0.028154 | 1.4081 | 0.49378 | 0.034378 |
| Hypotaurine | HILIC_NEG | C2 H7 N O2 S | 109.02005 | 8.22 | 85 | | 1.4563 | 0.28838 | 0.01241 | 1.8349 | 0.87568 | 5.31E−05 |
| Crotonic acid | HILIC_NEG | C4 H6 O2 | 86.03688 | 8.19 | 61.9 | | 1.4285 | 0.51455 | 0.000398 | 1.5042 | 0.58902 | 0.00014 |
| Lysylvaline | C18_POS | C11 H23 N3 O3 | 245.17367 | 0.934 | | | 1.4244 | 0.51037 | 0.006732 | 2.0971 | 1.0684 | 0.006152 |
| Methylglyoxal | C18_POS | C3 H4 O2 | 72.02108 | 1.019 | | | 1.4219 | 0.50777 | 0.001555 | 1.4003 | 0.48575 | 0.000958 |
| Pyruvic acid | HILIC_NEG | C3 H4 O3 | 88.01616 | 8.169 | 87.5 | | 1.4189 | 0.50475 | 0.003217 | 1.416 | 0.50182 | 0.005034 |
| (2R_3S)-2_3- | C18_POS | C6 H10 O5 | 162.05261 | 1.02 | | | 1.4149 | 0.50073 | 0.007619 | 1.4346 | 0.52065 | 0.002722 |
| Dimethylmalate | | | | | | | | | | | | |
| enolaldehyde | C18_POS | C3 H4 O2 | 72.02108 | 1.013 | | | 1.4148 | 0.50058 | 0.000686 | 1.5621 | 0.6435 | 0.000472 |
| Fructose 1,6- | HILIC_POS | C6 H14 O12 P2 | 339.99616 | 11.983 | | | 1.4138 | 0.44359 | 0.001835 | 1.5789 | 0.65891 | 0.000229 |
| diphosphate | | | | | | | | | | | | |
| Pseudouridine | HILIC_NEG | C9 H12 N2 O6 | 244.06952 | 6.725 | 88.9 | 54.1 | 1.4096 | 0.4953 | 0.009004 | 1.1851 | 0.24499 | 0.039456 |
| trans-4-hydroxy-L- | HILIC_POS | C7 H14 N O3 | 159.08983 | 6.948 | | | 1.4096 | 0.49524 | 0.015447 | 1.6343 | 0.70871 | 0.000282 |
| proline betaine | | | | | | | | | | | | |
| 2′,4′,6′- | C18_POS | C8 H8 O4 | 168.04195 | 1.005 | | | 1.4023 | 0.4878 | 0.008369 | 1.8094 | 0.85555 | 0.001137 |
| trihydroxyacetophen | | | | | | | | | | | | |
| one | | | | | | | | | | | | |
| 4H-pyran | C18_POS | C5 H6 O | 82.04182 | 1.026 | | | 1.3932 | 0.47841 | 0.005148 | 1.6875 | 0.75486 | 0.008907 |
| N2-Methylguanosine | HILIC_POS | C11 H15 N5 O5 | 297.10747 | 5.662 | 91.5 | | 1.3924 | 0.47757 | 0.01836 | 1.1954 | 0.25753 | 0.006945 |
| D-(−)-Arabinose | HILIC_NEG | C5 H10 O5 | 150.05305 | 8.2 | 85.4 | | 1.3829 | 0.46769 | 0.014238 | 1.3614 | 0.44514 | 0.008407 |
| L-(−)-Serine | C18_NEG | C3 H7 N O3 | 105.04262 | 0.934 | | | 1.3811 | 0.46579 | 0.015288 | 1.3326 | 0.4143 | 0.003844 |
| D-(+)-Maltose | HILIC_POS | C12 H22 O11 | 364.0984 | 9.54 | 94.5 | | 1.3793 | 0.37913 | 0.003581 | 1.9505 | 0.96381 | 0.000501 |
| Malondialdehyde | HILIC_NEG | C3 H4 O2 | 72.02141 | 7.939 | | | 1.3721 | 0.4564 | 0.021998 | 1.3574 | 0.44088 | 0.046394 |
| (S)-Methylmalonatesemialdehyde | C18_POS | C4 H6 O3 | 102.03165 | 1.031 | | | 1.3717 | 0.45594 | 0.039785 | 1.3312 | 0.41272 | 0.048284 |
| Diaminopimelic acid | HILIC_POS | C7 H14 N2 O4 | 190.09559 | 8.106 | 66.9 | | 1.3559 | 0.4392 | 0.007408 | 1.3738 | 0.45821 | 0.002862 |
| Glycolic acid | HILIC_NEG | C2 H4 O3 | 76.01594 | 7.548 | 99.2 | | 1.3381 | 0.30814 | 0.027255 | 1.3685 | 0.34309 | 0.025645 |
| 5-hydroxy-4- | C18_POS | C6 H8 O4 | 144.04212 | 1.02 | 61 | | 1.3331 | 0.41483 | 0.001962 | 1.3171 | 0.39736 | 0.00254 |
| methoxy-5,6- | | | | | | | | | | | | |
| dihydro-2H-pyran-2- | | | | | | | | | | | | |
| one | | | | | | | | | | | | |
| Adenine | HILIC_POS | C5 H5 N5 | 135.05478 | 9.697 | 75.5 | | 1.3249 | 0.40592 | 0.002822 | 1.1355 | 0.18336 | 0.039441 |
| 2-Oxobutyric acid | HILIC_NEG | C4 H6 O3 | 102.03195 | 7.486 | | | 1.3148 | 0.39485 | 0.000399 | 1.4693 | 0.55518 | 0.006556 |
| Levulinic acid | HILIC_NEG | C5 H8 O3 | 116.04761 | 7.486 | | | 1.3143 | 0.39426 | 0.000152 | 1.3428 | 0.42524 | 1.32E−05 |
| Ascorbyl glucoside | HILIC_NEG | C12 H18 O11 | 338.08498 | 8.924 | | | 1.3096 | 0.38917 | 0.011138 | 1.1958 | 0.25792 | 0.021363 |
| D-Glucosamine | HILIC_POS | C6 H13 N O5 | 162.05293 | 8.562 | 70 | | 1.3061 | 0.14549 | 0.02776 | 1.5547 | 0.63659 | 0.021802 |
| N6,N6,N6- | HILIC_POS | C9 H20 N2 O2 | 188.15281 | 10.789 | 93.4 | 95.5 | 1.2973 | 0.37549 | 0.009825 | 1.2258 | 0.2937 | 0.013161 |
| Trimethyl-L-lysine | | | | | | | | | | | | |
| D-(−)-Fructose | HILIC NEG | C6 H12 O6 | 180.06375 | 7.456 | 92.2 | | 1.2806 | 0.23948 | 0.003083 | 1.307 | 0.38623 | 0.000897 |
| 2-Oxovalericacid | C18_POS | C5 H8 O3 | 116.04725 | 1.026 | | | 1.2787 | 0.35469 | 0.047828 | 1.3332 | 0.41492 | 0.031689 |
| 1,2-benzoquinone | C18_POS | C6 H4 O2 | 108.02111 | 1.017 | | | 1.2769 | 0.35259 | 0.004356 | 1.5338 | 0.61713 | 0.000429 |
| [Similar to: D-(+)- | C18_NEG | C7 H10 N2 O4 S | 218.03663 | 0.991 | | | 1.2741 | 0.34943 | 0.02027 | 1.1266 | 0.17196 | 0.023302 |
| Galactose; ΔMass: | | | | | | | | | | | | |
| −37.9732 Da] | | | | | | | | | | | | |
| trans-Zeatin | HILIC_POS | C10 H13 N5 O | 219.11084 | 6.44 | 43.6 | 91.7 | 1.271 | 0.17757 | 0.00184 | 1.3281 | 0.40941 | 0.008021 |
| Bis-D- | C18_POS | C12 H20 O10 | 324.10493 | 1.015 | | | 1.2657 | 0.33994 | 0.015843 | 1.3876 | 0.47256 | 0.000589 |
| fructose2′_1:2_1′- | | | | | | | | | | | | |
| dianhydride | | | | | | | | | | | | |
| Glutaric acid | HILIC_NEG | C5 H8 O4 | 132.04257 | 7.477 | | | 1.2638 | 0.33776 | 0.002641 | 1.4051 | 0.49068 | 0.021986 |
| Glutaric acid | C18_NEG | C5 H8 O4 | 132.04219 | 0.999 | | | 1.2632 | 0.33703 | 0.03335 | 1.3925 | 0.47765 | 0.002326 |
| 1_2-Benzoquinone | C18_POS | C6 H4 O2 | 108.02106 | 1.023 | | | 1.2469 | 0.3184 | 0.011375 | 1.3447 | 0.42727 | 0.002194 |
| D-(−)-Fructose | C18_NEG | C6 H12 O6 | 120.04213 | 0.997 | 85 | | 1.2301 | 0.29874 | 0.035999 | 1.4416 | 0.52771 | 0.010484 |
| DL-Lactic Acid | C18_NEG | C3 H6 O3 | 90.03156 | 0.997 | 67.5 | | 1.2219 | 0.28918 | 0.043214 | 1.4409 | 0.52693 | 0.007205 |
| his-gln | C18_POS | C11 H17 N5 O4 | 283.12695 | 0.984 | | | 1.2196 | 0.28647 | 0.016343 | 1.4568 | 0.54282 | 0.004828 |
| 4-hyroxy-5-methyl- | C18_NEG | C5 H6 O3 | 114.03169 | 0.996 | | | 1.214 | 0.27981 | 0.033153 | 1.3309 | 0.41241 | 0.002751 |
| 3-furanone | | | | | | | | | | | | |
| (2R)-2,3- | HILIC_NEG | C3 H6 O4 | 106.02686 | 7.866 | 70.5 | 94 | 1.2125 | 0.27793 | 0.021863 | 1.1961 | 0.25829 | 0.016298 |
| Dihydroxypropanoic | | | | | | | | | | | | |
| acid | | | | | | | | | | | | |
| [Similar to: α,α- | C18_NEG | C12 H23 Cl O11 | 378.09224 | 1.042 | | | 1.2082 | 0.27289 | 0.044863 | 1.2735 | 0.34876 | 0.039251 |
| Trehalose; ΔMass: | | | | | | | | | | | | |
| −35.9760 Da] | | | | | | | | | | | | |
| D-(−)-Erythrose | HILIC_NEG | C4 H8 O4 | 120.04256 | 7.479 | | | 1.2032 | 0.26687 | 0.002052 | 1.3217 | 0.4024 | 0.001356 |
| Glucoheptonic Acid | HILIC_NEG | C7 H14 O8 | 226.06904 | 7.508 | | | 1.1567 | 0.21004 | 0.025905 | 1.2301 | 0.29872 | 0.006079 |
| 1-O-4- | HILIC NEG | C13 H16 O8 | 300.08449 | 8.653 | | | 1.1035 | 0.14206 | 0.014905 | 1.5051 | 0.58982 | 0.00302 |
| hydroxybenzoyl-β-D- | | | | | | | | | | | | |
| glucose | | | | | | | | | | | | |
| Threonine | HILIC_NEG | C4 H9 N O3 | 119.0586 | 8.036 | 85.1 | | 0.95167 | −0.07147 | 0.043555 | 0.67401 | −0.56915 | 2.89E−05 |
| Citric acid | HILIC_NEG | C6 H8 O7 | 192.02729 | 11.06 | 94.8 | | 0.87375 | −0.19471 | 0.032342 | 0.8134 | −0.29796 | 0.00302 |
| L-citrulline | HILIC_NEG | C6 H13 N3 O3 | 175.09585 | 8.747 | | 67.4 | 0.85117 | −0.23247 | 0.016699 | 0.32732 | −1.6112 | 1.49E−05 |
| L-Aspartic acid | C18_NEG | C4 H7 N O4 | 133.03746 | 0.951 | 85.7 | 91.5 | 0.84212 | −0.2479 | 0.03446 | 0.85285 | −0.22964 | 0.031889 |
| Ornithine | HILIC_NEG | C5 H12 N2 O2 | 132.09016 | 12.11 | 87.1 | | 0.83781 | −0.2553 | 0.023258 | 0.32523 | −1.6205 | 1.08E−05 |
| L-alanyl-L-alaninc | C18_POS | C6 H12 N2 O3 | 160.08459 | 1.033 | | | 0.83112 | −0.26686 | 0.001243 | 0.73428 | −0.4456 | 0.00053 |
| 4-Oxoproline | HILIC_NEG | C5 H7 N O3 | 129.04292 | 7.288 | 88.8 | | 0.82416 | −0.27901 | 0.014221 | 0.43901 | −1.1877 | 6.26E−05 |
| 2-[(2-Amino-2- | HILIC_NEG | C7 H11 N O6 S | 237.03077 | 10.625 | | | 0.81433 | −0.29631 | 0.0397 | 0.81569 | −0.29391 | 0.045862 |
| carboxyethyl)thio]butanedioic | | | | | | | | | | | | |
| acid | | | | | | | | | | | | |
| Glycerol 3-phosphate | HILIC_NEG | C3 H9 O6 P | 172.01393 | 9.33 | 71 | | 0.81312 | −0.29847 | 0.003221 | 0.91699 | −0.12502 | 0.035111 |
| 4-Hydroxybenzaldehyde | C18_POS | C7 H6 O2 | 122.03697 | 2.54 | 98.2 | | 0.81025 | −0.30356 | 0.039472 | 0.63402 | −0.65741 | 0.000507 |
| sn-glycero-3- | C18_POS | C8 H20 N O6 P | 257.10286 | 0.98 | | | 0.8083 | −0.30704 | 0.004286 | 0.77406 | −0.36948 | 0.019742 |
| Phosphocholine | | | | | | | | | | | | |
| L-(+)-Alanine | C18_NEG | C3 H7 N O2 | 89.04755 | 0.949 | | | 0.80505 | −0.31285 | 0.043739 | 0.8211 | −0.28437 | 0.04555 |
| 2-Amino-4- | HILIC_POS | C5 H7 N3 | 92.0377 | 8.234 | 84.5 | | 0.80303 | −0.28098 | 0.035145 | 0.53383 | −0.90555 | 0.000155 |
| methylpyrimidine | | | | | | | | | | | | |
| Oxalic acid | HILIC_NEG | C2 H2 O4 | 89.99547 | 10.751 | | | 0.79107 | −0.33812 | 0.001802 | 0.79922 | −0.32333 | 0.001602 |
| trans-Aconitic acid | HILIC_NEG | C6 H6 O6 | 174.01666 | 11.643 | 83.6 | 76.6 | 0.78541 | −0.34849 | 0.013483 | 0.68721 | −0.5265 | 0.002396 |
| Y-Aminobutyric acid | HILIC_NEG | C4 H9 N O2 | 103.06362 | 8.519 | 87.9 | | 0.78506 | −0.34913 | 0.002556 | 0.88078 | −0.18315 | 0.027693 |
| (GABA) | | | | | | | | | | | | |
| Histamine | C18_POS | C5 H9 N3 | 111.07976 | 0.852 | 99.3 | | 0.77781 | −0.36252 | 0.003384 | 0.44653 | −1.1632 | 4.14E−05 |
| N~6~-Methyl-L- | HILIC_POS | C7 H16 N2 O2 | 160.12145 | 11.319 | | | 0.77723 | −0.36358 | 0.020598 | 0.4443 | −1.1704 | 0.000202 |
| lysine | | | | | | | | | | | | |
| 1-deoxy-L-glycero- | C18_POS | C4 H9 O6 P | 181.998 | 1.325 | | | 0.77014 | −0.3768 | 0.012401 | 0.8311 | −0.26691 | 0.008107 |
| tetrulose 4-phosphate | | | | | | | | | | | | |
| 2-Hydroxy-2_4- | C18_POS | C5 H6 O3 | 114.03168 | 9.564 | | | 0.76482 | −0.38681 | 0.009344 | 0.84766 | −0.23845 | 0.013235 |
| pentadienoate | | | | | | | | | | | | |
| N-Stearoyl-L- | C18_POS | C27 H45 N O3 | 431.3401 | 9.44 | | | 0.76353 | −0.38925 | 0.02006 | 0.80527 | −0.31246 | 0.020583 |
| phenylalanine | | | | | | | | | | | | |
| 2-iminopropanoate | C18_POS | C3 H5 N O2 | 87.03193 | 1.019 | | | 0.76253 | −0.39113 | 0.011902 | 0.75916 | −0.39753 | 0.021272 |
| 4-hydroxycoumarin | C18_POS | C9 H6 O3 | 162.03198 | 7.976 | | | 0.761 | −0.39404 | 0.02811 | 0.64421 | −0.63439 | 0.010557 |
| DL-Glutamine | C18_POS | C5 H10 N2 O3 | 146.06935 | 0.947 | 99.9 | | 0.7608 | −0.39442 | 0.003343 | 0.58399 | −0.5835 | 0.001385 |
| N5-(L-1- | C18_POS | C8 H16 N2 O4 | 204.11072 | 1.043 | | | 0.75745 | −0.32653 | 0.044088 | 0.87887 | −0.18628 | 0.027825 |
| Carboxyethyl)-L- | | | | | | | | | | | | |
| ornithine | | | | | | | | | | | | |
| (4R)-5-Hydroxy-L- | C18_POS | C6 H13 NO3 | 147.08961 | 1.069 | | | 0.75669 | −0.40222 | 0.024785 | 0.69508 | −0.52475 | 0.003751 |
| leucine | | | | | | | | | | | | |
| Pipecolic acid | C18_POS | C6 H11 N O2 | 129.07916 | 1.069 | 54.7 | | 0.75604 | −0.40347 | 0.001991 | 0.78771 | −0.34426 | 0.010557 |
| N-Benzylformamide | C18_POS | C8 H9 N O | 118.04211 | 2.541 | 69.9 | | 0.75269 | −0.40987 | 0.045091 | 0.65196 | −0.61714 | 0.022646 |
| DL-Arginine | HILIC_NEG | C6 H14 N4 O2 | 174.1119 | 12.11 | 85.4 | | 0.74971 | −0.4156 | 0.002308 | 0.31081 | −1.6859 | 5.15E−06 |
| L-cysteinyl-glycine | C18_POS | C5 H10 N2 O3 S | 178.04143 | 1.683 | | | 0.74854 | −0.41786 | 0.039346 | 0.71199 | −0.49007 | 0.004175 |
| L-Glutamic acid | HILIC_NEG | C5 H9 N O4 | 147.05348 | 8.931 | 85.1 | | 0.74714 | −0.42055 | 0.000137 | 0.66713 | −0.58395 | 8.77E−05 |
| D-Pipecolicacid | C18_POS | C6 H11 N O2 | 129.07921 | 0.868 | | | 0.74613 | −0.35772 | 0.009718 | 0.4497 | −1.153 | 5.24E−05 |
| L-alanyl-L-alanine | HILIC_POS | C6 H12 N2 O3 | 160.08504 | 7.678 | | | 0.74613 | −0.42251 | 0.022478 | 0.55654 | −0.84545 | 0.000249 |
| L-Lysine | HILIC_POS | C6 H14 N2 O2 | 146.10581 | 11.872 | 92.1 | | 0.74207 | −0.43038 | 0.016901 | 0.42279 | −1.242 | 7.03E−05 |
| D-(+)-Pipecolinic | HILIC_POS | C6 H11 N O2 | 129.07928 | 7.016 | 95.6 | | 0.74053 | −0.43336 | 0.000726 | 0.69803 | −0.51864 | 4.38E−05 |
| acid | | | | | | | | | | | | |
| (S)-2-Acetolactate | C18_POS | C5 H8 O4 | 132.04232 | 9.559 | | | 0.74019 | −0.43404 | 0.012648 | 0.84387 | −0.24491 | 0.009238 |
| L-Histidine | HILIC_POS | C6 H9 N3 O2 | 155.0698 | 8.231 | 96.4 | | 0.73851 | −0.4373 | 0.002822 | 0.5604 | −0.83548 | 7.00E−05 |
| pyridoxine | HILIC_POS | C8 H11 N O3 | 169.07409 | 8.596 | | | 0.73851 | −0.43732 | 0.002948 | 0.67735 | −0.56203 | 0.000219 |
| N-Acetylornithine | HILIC_POS | C7 H14 N2 O3 | 174.10069 | 7.782 | 79 | | 0.73723 | −0.43981 | 0.011151 | 0.46176 | −1.1148 | 0.00013 |
| DL-Lysine | C18_NEG | C6 H14 N2 O2 | 146.10535 | 0.916 | | | 0.73286 | −0.4484 | 0.035488 | 0.40621 | −1.2997 | 0.005602 |
| L-Histidine | C18_POS | C6 H9 N3 O2 | 155.06969 | 0.935 | 84.4 | 59.9 | 0.72855 | −0.45691 | 0.017015 | 0.58308 | −0.77823 | 0.002217 |
| L-isoleucyl-glycine | C18_POS | C8 H16 N2 O3 | 188.11622 | 1.463 | | | 0.72597 | −0.46202 | 0.032081 | 0.54972 | −0.86324 | 0.018028 |
| Valylvaline | C18_POS | C10 H20 N2 O3 | 216.14764 | 9.128 | | | 0.72319 | −0.46755 | 0.011944 | 0.86098 | −0.21596 | 0.023517 |
| D-Glucosamine 6- | HILIC_NEG | C6 H14 N O8 P | 259.04559 | 9.317 | | | 0.72256 | −0.46882 | 0.002196 | 0.78114 | −0.35635 | 0.012044 |
| phosphate | | | | | | | | | | | | |
| nicotinamide | C18 POS | C6 H6 N2 O | 122.04778 | 1.695 | 96.9 | | 0.72149 | −0.47096 | 0.00842 | 0.6758 | −0.56534 | 0.018456 |
| N~2~-[2- | HILIC_POS | C12 H20 N4 O8 | 348.12818 | 10.383 | | | 0.72118 | −0.47158 | 0.020796 | 0.40823 | −1.2925 | 0.000248 |
| (Carboxymethyl)-2- | | | | | | | | | | | | |
| hydroxy-3-methoxy- | | | | | | | | | | | | |
| 3-oxopropanoyl]arginine | | | | | | | | | | | | |
| 2-Amino-2-deoxy-6- | HILIC_NEG | C6 H14 N O8 P | 259.04559 | 9.299 | | | 0.71577 | −0.48244 | 0.001564 | 0.78298 | −0.35295 | 0.013568 |
| O-phosphono-D- | | | | | | | | | | | | |
| glucopyranose | | | | | | | | | | | | |
| Cysteinylglycine(Cys- | C18_POS | C5 H10 N2 O3 S | 178.04144 | 1.686 | | | 0.71164 | −0.49077 | 0.018392 | 0.66913 | −0.57965 | 0.014432 |
| Gly) | | | | | | | | | | | | |
| 4-methyl-3- | HILIC_NEG | C7 H10 O5 | 172.03741 | 7.914 | | | 0.71159 | −0.49088 | 0.002803 | 0.77584 | −0.36617 | 0.003815 |
| oxoadipate | | | | | | | | | | | | |
| pyrimidine | HILIC_POS | C4 H4 N2 | 80.03759 | 7.825 | | | 0.70835 | −0.49747 | 0.002822 | 0.61349 | −0.7049 | 0.000302 |
| S-methyl-5′- | C18_POS | C11 H15 N5 O3 S | 297.08978 | 8.452 | | | 0.70697 | −0.50028 | 0.010361 | 0.8067 | −0.3099 | 0.042765 |
| thioadenosine | | | | | | | | | | | | |
| asp-gln | C18_POS | C9 H15 N3 O6 | 261.0962 | 0.998 | | | 0.70647 | −0.50131 | 0.026254 | 0.55451 | −0.85073 | 0.008754 |
| Methionine | HILIC_POS | C5 H11 N O2 S | 132.02475 | 6.788 | 85.2 | 92.7 | 0.70623 | −0.50179 | 0.020019 | 0.47838 | −1.0638 | 0.000202 |
| nicotinamide | HILIC_POS | C6 H6 N2 O | 122.04817 | 9.039 | | | 0.70614 | −0.50198 | 0.004909 | 0.6607 | −0.59794 | 0.001359 |
| 4-Methylene-L- | HILIC_NEG | C6 H9 N O4 | 159.05339 | 5.758 | | | 0.70558 | −0.50312 | 0.008486 | 0.67694 | −0).5629 | 0.002359 |
| glutamic acid | | | | | | | | | | | | |
| glu-asp | HILIC_NEG | C9 H14 N2 O7 | 262.08004 | 9.169 | | | 0.70311 | −0.50817 | 0.004223 | 0.72932 | −0.45537 | 0.005176 |
| N-Acetyl-L- | HILIC_NEG | C11 H13 N O3 | 207.08962 | 7.911 | 54.9 | | 0.70271 | −0.50899 | 0.000591 | 0.76064 | −0.39472 | 0.003868 |
| phenylalanine | | | | | | | | | | | | |
| phosphoglycolohydroxamate | C18_POS | C2 H6 N O6 P | 170.99358 | 0.926 | | | 0.70261 | −0.50921 | 0.004091 | 0.70874 | −0.49667 | 0.001007 |
| L-Aspartic acid | HILIC_NEG | C4 H7 N O4 | 133.03781 | 9.045 | 83.2 | 93.7 | 0.69951 | −0.51558 | 2.17E−05 | 0.5585 | −0.84037 | 5.31E−05 |
| Methylmalonic acid | HILIC_NEG | C4 H6 O4 | 118.02692 | 9.617 | 86.6 | 90.2 | 0.69796 | −0.51879 | 0.001046 | 0.6371 | −0.65042 | 0.000738 |
| asn-pro | HILIC_POS | C9 H15 N3 O4 | 229.10645 | 8.493 | | | 0.69788 | −0.51896 | 0.003477 | 0.4967 | −1.0096 | 7.03E−05 |
| L-Glutamic acid | HILIC_POS | C5 H9 N O4 | 147.05341 | 9.069 | 96.1 | 95.8 | 0.69732 | −0.52011 | 0.01415 | 0.69473 | −0.52548 | 0.000856 |
| (.+/−.)-Tartaric acid | HILIC_NEG | C4 H6 O6 | 150.01666 | 10.537 | | | 0.69697 | −0.52083 | 0.026201 | 0.75628 | −0.403 | 0.008487 |
| 4-Acetamidobutanoic | HILIC_POS | C6 H11 NO3 | 145.07413 | 8.584 | 42.7 | | 0.69176 | −0.53166 | 0.02169 | 0.58028 | −0.78518 | 0.003214 |
| acid | | | | | | | | | | | | |
| L-Glutathione | HILIC_POS | C10 H17 N3 O6 S | 307.08392 | 9.045 | 94.2 | 95.5 | 0.68777 | −0.54 | 0.030047 | 0.7764 | −0.36513 | 0.004022 |
| (reduced) | | | | | | | | | | | | |
| L-Aspartic acid | HILIC_POS | C4 H7 N O4 | 133.03774 | 9.203 | 99.6 | | 0.68666 | −0.54233 | 0.006698 | 0.63204 | −0.66191 | 0.000634 |
| (2R,3S,4S,5R,6R)-2- | C18_POS | C21 H36 O10 | 465.25775 | 9.711 | 93.9 | | 0.68423 | −0.54744 | 0.006113 | 0.70153 | −0.51142 | 0.009238 |
| ({[(2R,3R,4R,5S)- | | | | | | | | | | | | |
| 3,4-dihydroxy-5- | | | | | | | | | | | | |
| (hydroxymethyl)oxolan- | | | | | | | | | | | | |
| 2-yl]oxy}methyl)- | | | | | | | | | | | | |
| 6-{[(2E)-3,7- | | | | | | | | | | | | |
| dimethylocta-2,6- | | | | | | | | | | | | |
| dien-1-yl]oxy}oxane- | | | | | | | | | | | | |
| 3,4,5-triol | | | | | | | | | | | | |
| β-Alanine | HILIC NEG | C3 H7 N O2 | 89.0478 | 9.045 | 93 | 81.6 | 0.68372 | −0.54853 | 0.000137 | 0.56067 | −0.83478 | 0.000139 |
| L-gamma-Glutamyl- | HILIC_POS | C11 H20 N2 O5 | 260.13742 | 7.704 | | | 0.68363 | −0.54872 | 0.030047 | 0.46367 | −1.1088 | 0.002377 |
| L-leucine | | | | | | | | | | | | |
| benzaldehyde | C18_POS | C7 H6 O | 106.042 | 2.554 | | | 0.6829 | −0.55026 | 0.008413 | 0.55445 | −0.85087 | 0.030514 |
| L-Tyrosine | C18_POS | C9 H11 N O3 | 181.07425 | 2.546 | 99.8 | 90.4 | 0.6814 | −0.55344 | 0.009413 | 0.65496 | −0.61051 | 0.004136 |
| gamma-Glu-Ala | C18_POS | C8 H14 N2 O5 | 218.09043 | 1.458 | | | 0.68137 | −0.55349 | 0.006628 | 0.56495 | −0.8238 | 0.008907 |
| Cytidine | HILIC_NEG | C9 H13 N3 O5 | 243.08739 | 8.101 | 37.4 | | 0.68089 | −0.5545 | 0.003767 | 0.70921 | −0.49571 | 0.006577 |
| (R)-Lactaldehyde | C18 POS | C3 H6 O2 | 74.0367 | 1.042 | | | 0.68084 | −0.55461 | 0.000452 | 0.54252 | −0.88226 | 8.18E−05 |
| N-acetyl-(L)-arginine | C18_POS | C8 H16 N4 O3 | 216.12245 | 1.504 | | | 0.67912 | −0.55826 | 0.013264 | 0.62432 | −0.67963 | 0.02867 |
| L-Glutathione | HILIC_NEG | C10 H17 N3 O6 S | 307.0839 | 8.913 | 85.7 | | 0.67841 | −0.55976 | 0.002556 | 0.70782 | −0.49854 | 0.008188 |
| (reduced) | | | | | | | | | | | | |
| Maleamic acid | HILIC_POS | C4 H5 N O3 | 115.02715 | 9.205 | | | 0.67832 | −0.55995 | 0.001911 | 0.62008 | −0.68948 | 0.000284 |
| D-(+)-Proline | HILIC_POS | C5 H9 N O2 | 115.06351 | 7.17 | 96.8 | 86.4 | 0.67577 | −0.56539 | 0.006497 | 0.48824 | −1.0344 | 7.00E−05 |
| Asparagine | HILIC_POS | C4 H8 N2 O3 | 132.05372 | 8.58 | 94.8 | 93.1 | 0.67412 | −0.56892 | 0.007846 | 0.54493 | −0.87587 | 0.001676 |
| 1-Pyrroline | C18_POS | C4 H7 N | 69.05786 | 1.042 | | | 0.67239 | −0.57262 | 0.001853 | 0.47958 | −1.0602 | 9.66E−05 |
| DL-β- | C18_POS | C5 H11 NO3 | 133.0738 | 1.037 | | | 0.67166 | −0.5742 | 0.000449 | 0.57554 | −0.79701 | 0.001222 |
| hydroxynorvaline | | | | | | | | | | | | |
| 3-Methylhistidine | HILIC_POS | C7 H11 N3 O2 | 169.08536 | 7.674 | 93.2 | | 0.6679 | −0.5823 | 0.006497 | 0.51412 | −0.95983 | 0.000113 |
| phenol | C18_POS | C6 H6 O | 94.04213 | 2.54 | | | 0.66597 | −0.58647 | 0.017107 | 0.63894 | −0.64625 | 0.014124 |
| 2-iminopropanoate | HILIC_POS | C3 H5 N O2 | 87.03218 | 9.204 | | | 0.66122 | −0.59679 | 0.001452 | 0.60252 | −0.73092 | 9.07E−05 |
| DL-Lysine | HILIC_NEG | C6 H14 N2 O2 | 146.1058 | 11.803 | | | 0.65872 | −0.60226 | 0.008689 | 0.32428 | −1.6247 | 0.000104 |
| Asparagine | HILIC_NEG | C4 H8 N2 O3 | 132.05372 | 8.463 | 80.4 | | 0.65743 | −0.6051 | 0.002084 | 0.40452 | −1.3057 | 4.87E−05 |
| 5′-S-Methyl-5′- | HILIC_POS | C11 H15 N5 O3 | 297.09371 | 9.232 | 46 | | 0.65685 | −0.60636 | 0.002532 | 0.35908 | −1.4776 | 0.000351 |
| thioadenosine | | S C5 H9 N O2 | | | | | | | | | | |
| D-proline | C18_POS | | 115.06332 | 1.036 | | | 0.65455 | −0.61142 | 0.000255 | 0.51943 | −0.80411 | 0.000343 |
| D-idarate | HILIC_NEG | C6 H10 O8 | 208.02208 | 10.226 | | | 0.65415 | −0.6123 | 0.009237 | 0.63577 | −0.65341 | 0.020223 |
| Gluconic acid | HILIC_NEG | C6 H12 O7 | 196.05851 | 8.833 | 94.3 | 59.7 | 0.64985 | −0.62182 | 0.000707 | 0.81307 | −0.29856 | 0.037625 |
| Mevalonic acid | HILIC_NEG | C6 H12 O4 | 148.07361 | 8.327 | 53.9 | | 0.6464 | −0.6295 | 0.000599 | 0.43972 | −1.1853 | 2.89E−05 |
| D-(−)-Glutamine | HILIC_NEG | C5 H10 N2 O3 | 146.06944 | 8.328 | 81.2 | | 0.64549 | −0.63154 | 0.000211 | 0.44817 | −1.1579 | 5.15E−06 |
| (9S,13S)-12- | C18_POS | C18 H28 O3 | 292.20403 | 10.513 | | | 0.63616 | −0.65254 | 0.00384 | 0.72919 | −0.45564 | 0.009155 |
| Oxophytodicnoic | | | | | | | | | | | | |
| acid | | | | | | | | | | | | |
| asp-gln | HILIC_NEG | C9 H15 N3 O6 | 261.096 | 9.405 | | | 0.63122 | −0.66378 | 0.01241 | 0.43284 | −1.2081 | 0.001045 |
| Uridine | HILIC_NEG | C9 H13 N2 O9 P | 324.03587 | 9.463 | 88 | | 0.63052 | −0.66539 | 0.010889 | 0.71438 | −0.48523 | 0.016695 |
| monophosphate | | | | | | | | | | | | |
| (UMP) | | | | | | | | | | | | |
| L-Pyroglutamic acid | HILIC_POS | C5 H7 N O3 | 129.04287 | 9.067 | 91.1 | 88.6 | 0.62646 | −0.6747 | 0.001452 | 0.57011 | −0.81068 | 0.002321 |
| Tomatidine | C18_POS | C27 H45 N O2 | 415.34508 | 9.565 | 92.1 | | 0.6213 | −0.68664 | 0.001766 | 0.82411 | −0.27909 | 0.012621 |
| D-(+)-Pipecolinic | C18_POS | C6 H11 N O2 | 129.07915 | 1.443 | 94.8 | | 0.617 | −0.69665 | 0.015025 | 0.57934 | −0.78751 | 0.008365 |
| acid | | | | | | | | | | | | |
| glycyl-L-valine | C18_POS | C7 H14 N2 O3 | 174.10049 | 1.041 | | | 0.61651 | −0.69779 | 0.021253 | 0.28561 | −1.8079 | 0.038285 |
| asp-gln | HILIC_POS | C9 H15 N3 O6 | 261.09626 | 9.541 | | | 0.61457 | −0.70234 | 0.0154 | 0.39995 | −1.3221 | 0.00096 |
| N-(4-Amino-1- | HILIC_POS | C10 H18 N2 O6 | 262.11665 | 9.212 | | | 0.6137 | −0.7044 | 0.00411 | 0.57592 | −0.79607 | 0.001551 |
| carboxybutyl)glutamic | | | | | | | | | | | | |
| acid | | | | | | | | | | | | |
| L-prolyl-trans-4- | C18_POS | C10 H16 N2 O4 | 228.11124 | 0.988 | | | 0.60459 | −0.72596 | 0.014626 | 0.50951 | −0.97282 | 0.014988 |
| hydroxy-L-proline | | | | | | | | | | | | |
| (3beta,5alpha,25S)- | C18_POS | C33 H55 N O7 | 577.3981 | 9.565 | | | 0.60227 | −0.73152 | 0.001567 | 0.82056 | −0.28532 | 0.01063 |
| Spirosolan-3-yl beta- | | | | | | | | | | | | |
| D-galactopyranoside | | | | | | | | | | | | |
| 4-Oxoproline | C18_NEG | C5 H7 N O3 | 129.04259 | 1.92 | 87.7 | | 0.60047 | −0.73583 | 0.009704 | 0.41846 | −1.2568 | 0.02163 |
| Phosphoric acid | HILIC_NEG | H3 O4 P | 97.97708 | 9.638 | | 91 | 0.60017 | −0.73656 | 0.000178 | 0.66029 | −0.59882 | 0.000124 |
| 4-Pyridoxic acid | HILIC_NEG | C8 H9 N O4 | 183.0533 | 2.462 | 94.5 | | 0.59371 | −0.75217 | 0.002354 | 0.63901 | −0.64609 | 0.012017 |
| L-Pyroglutamic acid | C18_POS | C5 H7 N O3 | 129.04283 | 1.801 | 94.7 | | 0.58856 | −0.76475 | 0.048637 | 0.38805 | −1.3657 | 0.005241 |
| (E)-p-coumaric acid | C18_NEG | C9 H8 O3 | 164.04723 | 9.601 | | | 0.58305 | −0.7783 | 0.028081 | 0.62934 | −0.66809 | 0.023263 |
| geranyl 6-O-β-D- | HILIC NEG | C21 H36 O10 | 448.23091 | 2.725 | | | 0.5766 | −0.79437 | 0.044009 | 0.5133 | −0.96213 | 0.037665 |
| xylopyranosyl-β-D- | | | | | | | | | | | | |
| glucopyranoside | | | | | | | | | | | | |
| Isocitric acid | C18_NEG | C6 H8 O7 | 192.02681 | 1.341 | 97.5 | 96.8 | 0.5672 | −0.81808 | 0.015288 | 0.49602 | −1.0115 | 0.045351 |
| 4-Pyridoxic acid | C18_POS | C8 H9 N O4 | 183.05345 | 4.948 | 97.9 | | 0.55322 | −0.85407 | 0.060742 | 0.61622 | −0.69847 | 0.019742 |
| Pyrophosphoric Acid | HILIC_NEG | H4 O7 P2 | 177.94348 | 9.651 | | | 0.5512 | −0.85935 | 0.000143 | 0.57064 | −0.80934 | 0.000108 |
| L-Threonic acid | HILIC_NEG | C4 H8 O5 | 136.03747 | 10.244 | 79.5 | 90.3 | 0.54845 | −0.86656 | 0.003994 | 0.5456 | −0.87409 | 0.004315 |
| O-Acetylserine | HILIC_NEG | C5 H9 N O4 | 147.05342 | 7.491 | 70.3 | | 0.54679 | −0.87095 | 0.00251 | 0.61207 | −0.70824 | 0.004162 |
| Cytidine; 1-beta- | C18_POS | C9 H13 N3 O5 | 243.08513 | 1.043 | | | 0.5418 | −0.88418 | 0.004672 | 0.32425 | −1.6248 | 0.000276 |
| delta-Ribofuranosyl- | | | | | | | | | | | | |
| Cytosine | | | | | | | | | | | | |
| Itaconic acid | HILIC_NEG | C5 H6 O4 | 130.02693 | 11.127 | 98.5 | 90.3 | 0.54113 | −0.88595 | 0.026267 | 0.4972 | −1.0081 | 0.019264 |
| gamma-Glu-gln | C18_NEG | C10 H17 N3 O6 | 275.11108 | 1.049 | | | 0.53817 | −0.89387 | 0.04371 | 0.29893 | −1.7421 | 0.003857 |
| I±-L-Glutamyl-L- | C18_POS | C10 H17 N3 O6 | 275.1113 | 1.04 | | | 0.53236 | −0.90952 | 0.004672 | 0.31799 | −1.653 | 0.000276 |
| glutamine | | | | | | | | | | | | |
| trp-lys | HILIC_NEG | C17 H24 N4 O3 | 332.18348 | 2.634 | | | 0.52453 | −0.9309 | 0.001445 | 0.68472 | −0.54642 | 0.015836 |
| gamma-Glu-gln | HILIC_NEG | C10 H17 N3 O6 | 275.1118 | 9.139 | | | 0.51103 | −0.96852 | 0.00371 | 0.29213 | −1.7753 | 0.000163 |
| Methylmalonic acid | C18_NEG | C4 H6 O4 | 118.02666 | 2.268 | 44 | 70.5 | 0.50405 | −0.98835 | 0.02027 | 0.59207 | −0.75615 | 0.023263 |
| β-L-arabinose 1- | HILIC_NEG | C5 H11 O8 P | 228.00381 | 7.762 | | | 0.50395 | −0.98865 | 0.028578 | 0.64164 | −0.64015 | 0.026304 |
| phosphate | | | | | | | | | | | | |
| 3-Phosphoglyceric | C18_NEG | C3 H7 O7 P | 185.9927 | 1.853 | 37.8 | | 0.5025 | −0.99279 | 0.03335 | 0.68504 | −0.54575 | 0.046445 |
| acid | | | | | | | | | | | | |
| dihydrocoumarin | HILIC_NEG | C9 H8 O2 | 148.05271 | 8.892 | | | 0.50119 | −0.99657 | 0.000171 | 0.47542 | −1.0727 | 8.16E−05 |
| trans-Aconitic acid | C18_NEG | C6 H6 O6 | 174.01625 | 1.358 | 69.2 | 67.1 | 0.49875 | −1.0036 | 0.00043 | 0.59224 | −0.34103 | 0.00983 |
| 5,6-dihydrothymine | C18_POS | C5 H8 N2 O2 | 128.05858 | 0.938 | | | 0.49096 | −1.0263 | 0.006324 | 0.43031 | −1.2165 | 0.005316 |
| Nicotinic acid | HILIC_POS | C6 H5 N O2 | 123.03225 | 5.74 | 47.4 | | 0.48642 | −1.0397 | 0.001452 | 0.67604 | −0.56483 | 0.003262 |
| L-(−)-Methionine | C18_POS | C5 H11 N O2 S | 149.05133 | 1.507 | 99.6 | 94.6 | 0.48229 | −1.052 | 0.031838 | 0.31043 | −1.6877 | 0.005896 |
| gamma-Glu-Thr | HILIC_POS | C9 H16 N2 O6 | 248.10091 | 9.061 | | | 0.47706 | −1.0678 | 0.00327 | 0.27614 | −1.8565 | 0.00013 |
| L-Phenylalanine | HILIC_NEG | C9 H11 N O2 | 165.07921 | 8.895 | 92.6 | | 0.47616 | −1.0705 | 0.000131 | 0.48474 | −1.0447 | 8.16E−05 |
| Spermidine | C18_POS | C7 H19 N3 | 128.13157 | 0.821 | 91.2 | | 0.47203 | −1.0831 | 0.028622 | 0.50884 | −0.97473 | 0.005862 |
| L-glyceraldehyde 3- | C18_POS | C3 H7 O6 P | 167.98255 | 1.851 | | | 0.47092 | −1.0864 | 0.000865 | 0.75072 | −0.41364 | 0.025598 |
| phosphate | | | | | | | | | | | | |
| gamma-Glutamyl- | C18_POS | C9 H16 N2 O5 | 232.10614 | 1.701 | | | 0.47026 | −1.0885 | 0.033938 | 0.5179 | −0.94926 | 0.022646 |
| gamma- | | | | | | | | | | | | |
| aminobutyrate | | | | | | | | | | | | |
| glu-tyr | C18_POS | C14 H18 N2 O6 | 310.11656 | 7.132 | | | 0.46729 | −1.0976 | 0.004239 | 0.27544 | −1.8602 | 0.000276 |
| L-gamma-Glutamyl- | C18_POS | C11 H20 N2 O5 | 260.13737 | 8.185 | | | 0.46044 | −1.1189 | 0.001282 | 0.30255 | −1.7248 | 0.000343 |
| L-leucine | | | | | | | | | | | | |
| Methionine | C18_POS | C5 H11 N O2 S | 149.05132 | 1.518 | 91.3 | | 0.45948 | −1.1219 | 0.011607 | 0.35732 | −1.4847 | 0.010557 |
| L-gamma-Glutamyl- | C18_NEG | C11 H20 N2 O5 | 260.13662 | 8.466 | | | 0.45831 | −1.1256 | 0.023051 | 0.38832 | −1.3647 | 0.017945 |
| L-leucine | | | | | | | | | | | | |
| Dehydro-L-ascorbic | HILIC_NEG | C6 H6 O6 | 174.01659 | 6.025 | | 60.1 | 0.4574 | −1.1285 | 0.005115 | 0.63427 | −0.65683 | 0.022663 |
| acid | | | | | | | | | | | | |
| DL-Phenylalanine | C18_NEG | C9 H11 N O2 | 165.07869 | 1.006 | | | 0.45503 | −1.136 | 0.015288 | 0.5664 | −0.82011 | 0.002751 |
| (3R)-β-phenylalanine | C18_POS | C9 H11 N O2 | 165.07927 | 1.019 | | | 0.45407 | −1.139 | 0.000158 | 0.69661 | −0.52157 | 0.004648 |
| fumarate | HILIC_NEG | C4 H4 O4 | 113.99548 | 6.003 | | | 0.44887 | −1.1556 | 0.000564 | 0.51096 | −0.96871 | 0.000333 |
| asn-pro | HILIC_NEG | C9 H15 N3 O4 | 229.10624 | 8.33 | | | 0.44447 | −1.1699 | 0.000419 | 0.2239 | −2.159 | 5.15E−06 |
| Methyl alpha- | C18_NEG | C14 H18 N2 O5 | 294.121 | 9.194 | | | 0.43506 | −1.2007 | 0.017907 | 0.27606 | −1.857 | 0.005993 |
| aspartylphenylalaninate | | | | | | | | | | | | |
| (R)-4-hydroxy-4- | HILIC_NEG | C6 H8 O6 | 174.01659 | 6.006 | | | 0.40585 | −1.301 | 0.000586 | 0.50469 | −0.98652 | 0.000492 |
| methyl-2- | | | | | | | | | | | | |
| oxoglutarate | | | | | | | | | | | | |
| Oxalosuccinic acid | C18_NEG | C6 H6 O7 | 190.01117 | 1.744 | | | 0.40195 | −1.3149 | 0.015288 | 0.63723 | −0.65011 | 0.039667 |
| gamma-L-glutamyl- | C18 NEG | C14 H18 N2 O6 | 310.11594 | 7.139 | | | 0.40027 | −1.321 | 0.019457 | 0.43184 | −1.2114 | 0.023756 |
| L-tyrosine | | | | | | | | | | | | |
| p-cymene | C18_POS | C10 H14 | 134.1097 | 9.603 | | | 0.39549 | −1.3383 | 0.000526 | 0.46504 | −1.1046 | 0.001754 |
| L-Glutathione | C18_NEG | C20 H32 N6 O12 S2 | 612.15081 | 3.568 | 81.3 | 91.7 | 0.31245 | −1.6783 | 0.015288 | 0.62572 | −0.67641 | 0.04888 |
| oxidized | | | | | | | | | | | | |
| Citramalic acid | HILIC_NEG | C5 H8 O5 | 148.03748 | 8.668 | | 68.3 | 0.28247 | −1.8238 | 0.04879 | 0.36167 | −1.4673 | 0.02781 |
| 2-Isopropylmalic | HILIC_NEG | C7 H12 O5 | 176.06864 | 8.333 | 92.4 | 86.9 | 0.23328 | −2.0999 | 0.000398 | 0.25516 | −1.9705 | 0.000319 |
| acid | | | | | | | | | | | | |
| 7-O-Phosphonohept- | HILIC_NEG | C7 H15 O10 P | 290.04029 | 9.841 | | | 0.044055 | −4.5046 | 2.29E−07 | 0.088836 | −3.4927 | 2.68E−05 |
| 2-ulose | | | | | | | | | | | | |

TABLE 8
Changes in Glycerolipids Identified Using LC-MS in adp Fruit

| | | | | | | | adp vs. CL | | | adp vs. WT | | |
|---|---|---|---|---|---|---|---|---|---|---|---|---|
| Tissue | Stage | Mode | Peak_ID | Lipid class | mz | RT (min) | FC | log2(FC) | ajusted p | FC | log2(FC) | ajusted p |
| Placenta | 6 dpa | POS | MGDG 32:0 | Monogalactosyldiacylglycerol | 748.5934529 | 9.224245 | 0.64018 | −0.64344 | 0.046779 | 0.53973 | −0.88968 | 0.005602 |
| Placenta | 20 dpa | NEG | SQDG 32:3 | Sulfoquinovosyl diacylglycerol | 787.4997841 | 5.329678 | 1.7607 | 0.81612 | 0.000478 | 1.6463 | 0.71926 | 0.000874 |
| Placenta | 20 dpa | POS | TAG 50:6 | Triacylglycerol | 840.7072541 | 13.62613 | 1.3987 | 0.48414 | 0.000245 | 1.9031 | 0.92839 | 0.002516 |
| Placenta | 20 dpa | NEG | SQDG 32:2 | Sulfoquinovosyl diacylglycerol | 789.5159361 | 5.76877 | 1.3693 | 0.45348 | 0.004597 | 1.4253 | 0.51129 | 0.01695 |
| Placenta | 20 dpa | NEG | SQDG 36:6 | Sulfoquinovosyl diacylglycerol | 837.4821293 | 5.145032 | 1.3507 | 0.43372 | 0.026141 | 1.3669 | 0.45094 | 0.02174 |
| Placenta | 20 dpa | POS | MGDG 34:3 | Monogalactosyldiacylglycerol | 770.5762785 | 7.97749 | 0.60852 | −0.71662 | 0.00234 | 0.62336 | −0.68186 | 0.030585 |
| Placenta | 20 dpa | POS | DGDG 32:3 | Digalactosyldiacylglycerol | 904.5989096 | 6.497954 | 0.57709 | −0.79312 | 0.006145 | 0.59228 | −0.75565 | 0.049944 |
| Placenta | 20 dpa | POS | DGDG 32:1 | Digalactosyldiacylglycerol | 908.6308191 | 7.657834 | 0.56911 | −0.81323 | 0.003031 | 0.5773 | −0.7926 | 0.033397 |
| Placenta | 20 dpa | POS | DGDG 34:3 | Digalactosyldiacylglycerol | 932.6299042 | 7.225584 | 0.53834 | −0.8934 | 0.000919 | 0.57957 | −0.78694 | 0.030141 |
| Placenta | 20 dpa | POS | MGDG 34:2 | Monogalactosyldiacylglycerol | 772.5928779 | 8.659586 | 0.53724 | −0.89636 | 0.000145 | 0.64787 | −0.62621 | 0.013369 |
| Placenta | 20 dpa | POS | DGDG 34:2 | Digalactosyldiacylglycerol | 934.6456211 | 7.877595 | 0.49006 | −1.029 | 0.000188 | 0.57491 | −0.79859 | 0.037915 |
| Placenta | 20 dpa | POS | MGDG 36:3 | Monogalactosyldiacylglycerol | 798.6083615 | 8.856581 | 0.48787 | −1.0354 | 0.00041 | 0.56124 | −0.83332 | 0.012591 |
| Placenta | 20 dpa | POS | DGDG 38:5 | Digalactosyldiacylglycerol | 984.6600594 | 6.735676 | 0.45772 | −1.1274 | 0.004363 | 0.59115 | −0.75839 | 0.021535 |
| Placenta | 20 dpa | NEG | SQDG 32:0 | Sulfoquinovosyl diacylglycerol | 793.513623 | 6.800176 | 0.40607 | −1.3002 | 0.002727 | 0.58386 | −0.7763 | 0.007893 |
| Placenta | 20 dpa | POS | MGDG 36:4 | Monogalactosyldiacylglycerol | 796.5917938 | 8.09914 | 0.40591 | −1.3008 | 0.000302 | 0.61051 | −0.7119 | 0.037915 |
| Placenta | 20 dpa | POS | DGDG 38:4 | Digalactosyldiacylglycerol | 986.6758223 | 7.355799 | 0.40003 | −1.3218 | 0.001222 | 0.50851 | −0.97566 | 0.016275 |
| Placenta | 20 dpa | POS | DGDG 36:2 | Digalactosyldiacylglycerol | 962.677015 | 8.877216 | 0.39326 | −1.3464 | 6.87E−05 | 0.64499 | −0.63265 | 0.041043 |
| Placenta | 20 dpa | POS | DGDG 32:2 | Digalactosyldiacylglycerol | 906.6149351 | 7.103352 | 0.38756 | −1.3675 | 0.005946 | 0.39273 | −1.3484 | 0.001807 |
| Placenta | 20 dpa | POS | DGDG 36:5 | Digalactosyldiacylglycerol | 956.6290688 | 6.736353 | 0.38628 | −1.3723 | 9.91E−05 | 0.56524 | −0.82307 | 0.030585 |
| Placenta | 20 dpa | POS | MGDG 32:0 | Monogalactosyldiacylglycerol | 748.5934529 | 9.224245 | 0.35238 | −1.5048 | 5.08E−05 | 0.44769 | −1.1594 | 0.001807 |
| Placenta | 20 dpa | POS | DGDG 36:4 | Digalactosyldiacylglycerol | 958.6445553 | 7.356304 | 0.32606 | −1.6168 | 6.57E−05 | 0.47219 | −1.0825 | 0.01901 |
| Placenta | 20 dpa | POS | DGDG 34:0 | Digalactosyldiacylglycerol | 938.6777283 | 9.405775 | 0.24477 | −2.0305 | 2.81E−05 | 0.34369 | −1.5408 | 0.001807 |
| Placenta | 20 dpa | POS | DGDG 32:0 | Digalactosyldiacylglycerol | 910.6460983 | 8.390417 | 0.18524 | −2.4325 | 5.08E−05 | 0.25081 | −1.9953 | 0.001506 |
| Placenta | MG | NEG | SQDG 36:6 | Sulfoquinovosyl diacylglycerol | 837.4821293 | 5.145032 | 1.4642 | 0.55015 | 0.043236 | 1.4528 | 0.53883 | 0.025428 |
| Placenta | MG | POS | MGDG 36:3 | Monogalactosyldiacylglycerol | 798.6083615 | 8.856581 | 0.59024 | −0.76064 | 0.002642 | 0.74615 | −0.42246 | 0.035085 |
| Placenta | MG | POS | PC 32:1 | Phosphatidylcholine | 732.5542264 | 7.743365 | 0.54934 | −0.86423 | 0.011553 | 0.59992 | −0.73716 | 0.001976 |
| Placenta | MG | NEG | SQDG 34:2 | Sulfoquinovosyl diacylglycerol | 817.5140252 | 6.399002 | 0.53798 | −0.89437 | 0.006699 | 0.68744 | −0.54069 | 0.035428 |
| Placenta | MG | NEG | SQDG 32:0 | Sulfoquinovosyl diacylglycerol | 793.513623 | 6.800176 | 0.49447 | −1.016 | 0.000897 | 0.56774 | −0.81669 | 0.02066 |
| Placenta | MG | NEG | PG 34:1 | Phosphatidylglycerol | 747.5179288 | 7.318514 | 0.40133 | −1.3171 | 0.000725 | 0.70785 | −0.49849 | 0.012553 |
| Placenta | MG | POS | DGDG 32:1 | Digalactosyldiacylglycerol | 908.6308191 | 7.657834 | 0.39991 | −1.3223 | 0.001744 | 0.45989 | −1.1206 | 0.011126 |
| Placenta | MG | POS | DGDG 34:3 | Digalactosyldiacylglycerol | 932.6299042 | 7.225584 | 0.39375 | −1.3446 | 0.002183 | 0.55964 | −0.83743 | 0.041386 |
| Placenta | MG | POS | DGDG 34:2 | Digalactosyldiacylglycerol | 934.6456211 | 7.877595 | 0.36013 | −1.4734 | 0.000641 | 0.56435 | −0.82534 | 0.011346 |
| Placenta | MG | POS | MGDG 32:0 | Monogalactosyldiacylglycerol | 748.5934529 | 9.224245 | 0.35872 | −1.4791 | 0.000199 | 0.60115 | −0.7342 | 0.025252 |
| Placenta | MG | POS | DGDG 36:4 | Digalactosyldiacylglycerol | 958.6445553 | 7.356304 | 0.33803 | −1.5648 | 0.001047 | 0.57039 | −0.80999 | 0.031692 |
| Placenta | MG | POS | DGDG 36:2 | Digalactosyldiacylglycerol | 962.677015 | 8.877216 | 0.31531 | −1.6652 | 0.001047 | 0.66286 | −0.59323 | 0.014164 |
| Placenta | MG | POS | DGDG 34:0 | Digalactosyldiacylglycerol | 938.6777283 | 9.405775 | 0.16955 | −2.5602 | 0.000199 | 0.40573 | −1.3014 | 0.009902 |
| Placenta | MG | POS | DGDG 32:0 | Digalactosyldiacylglycerol | 910.6460983 | 8.390417 | 0.14726 | −2.7636 | 0.000165 | 0.28802 | −1.7958 | 0.001325 |
| Placenta | RR | POS | DGDG 36:1 | Digalactosyldiacylglycerol | 964.692874 | 9.638398 | 1.8726 | 0.90502 | 0.035209 | 2.4885 | 1.3153 | 0.001635 |
| Placenta | RR | POS | MGDG 36:3 | Monogalactosyldiacylglycerol | 798.6083615 | 8.856581 | 1.6909 | 0.75775 | 0.015795 | 1.4582 | 0.54414 | 0.016375 |
| Placenta | RR | POS | TAG 60:5 | Triacylglycerol | 982.8794078 | 15.60353 | 1.6757 | 0.74474 | 0.018794 | 1.6596 | 0.73082 | 0.005012 |
| Placenta | RR | NEG | PS 40:2 | Phosphatidylserine | 842.5908886 | 8.164167 | 1.535 | 0.61826 | 0.004783 | 1.1732 | 0.23043 | 0.0103 |
| Placenta | RR | POS | MGDG 36:4 | Monogalactosyldiacylglycerol | 796.5917938 | 8.09914 | 1.4874 | 0.57284 | 0.043767 | 1.3669 | 0.4509 | 0.018965 |
| Placenta | RR | NEG | PG 34:0 | Phosphatidylglycerol | 749.533836 | 8.03903 | 1.4742 | 0.55989 | 0.002571 | 1.8053 | 0.85226 | 0.003919 |
| Placenta | RR | POS | DGDG 36:4 | Digalactosyldiacylglycerol | 958.6445553 | 7.356304 | 1.4596 | 0.54559 | 0.020377 | 1.4804 | 0.56594 | 0.000243 |
| Placenta | RR | NEG | PS 42:1 | Phosphatidylserine | 872.6393998 | 10.04242 | 1.4384 | 0.52446 | 0.002555 | 2.3176 | 1.2126 | 0.000357 |
| Placenta | RR | POS | MGDG 34:0 | Monogalactosyldiacylglycerol | 776.6247732 | 10.24769 | 1.382 | 0.46681 | 0.019601 | 1.5028 | 0.58765 | 0.011005 |
| Placenta | RR | POS | MGDG 36:2 | Monogalactosyldiacylglycerol | 800.6238267 | 9.680866 | 1.3335 | 0.41517 | 0.039364 | 1.8281 | 0.87036 | 0.001161 |
| Placenta | RR | POS | MGDG 36:1 | Monogalactosyldiacylglycerol | 802.6413022 | 10.45046 | 1.308 | 0.38736 | 0.027689 | 1.7996 | 0.8477 | 0.000695 |
| Placenta | RR | POS | PC 38:2 | Phosphatidylcholine | 814.6318604 | 10.04102 | 1.3067 | 0.3859 | 0.027071 | 2.3619 | 1.24 | 0.000402 |
| Placenta | RR | NEG | SQDG 32:1 | Sulfoquinovosyl diacylglycerol | 791.5319008 | 6.259773 | 1.2798 | 0.3559 | 0.046685 | 1.3333 | 0.41498 | 0.009588 |
| Placenta | RR | NEG | PS 40:0 | Phosphatidylserine | 846.6234936 | 9.765872 | 1.2713 | 0.34626 | 0.035501 | 1.8786 | 0.90967 | 0.00173 |
| Placenta | RR | NEG | PS 42:2 | Phosphatidylserine | 870.6226294 | 9.115818 | 1.217 | 0.28336 | 0.025644 | 3.5779 | 1.8391 | 0.000501 |
| Placenta | RR | NEG | PS 40:1 | Phosphatidylserine | 844.6075846 | 8.976524 | 1.1884 | 0.24901 | 0.01896 | 2.056 | 1.0398 | 0.001039 |
| Placenta | RR | POS | MGDG 36:6 | Monogalactosyldiacylglycerol | 792.5606238 | 6.815294 | 0.79513 | −0.33074 | 0.03106 | 0.71332 | −0.48739 | 0.008361 |
| Placenta | RR | POS | TAG 54:9 | Triacylglycerol | 890.7224362 | 13.35717 | 0.75927 | −0.39732 | 0.011927 | 0.45027 | −1.1511 | 0.000695 |
| Placenta | RR | NEG | PG 36:5 | Phosphatidylglycerol | 767.487021 | 5.773432 | 0.71264 | −0.48875 | 0.020728 | 0.74267 | −0.4292 | 0.01131 |
| Placenta | RR | NEG | SQDG 36:5 | Sulfoquinovosyl diacylglycerol | 839.4974664 | 5.549654 | 0.69513 | −0.52465 | 0.013285 | 0.6306 | −0.6652 | 0.002299 |
| Placenta | RR | POS | DGDG 36:6 | Digalactosyldiacylglycerol | 954.6136146 | 6.169967 | 0.69169 | −0.5318 | 0.045779 | 0.6667 | −0.58489 | 0.005078 |
| Placenta | RR | NEG | PS 40:5 | Phosphatidylserine | 836.5445562 | 6.222667 | 0.68495 | −0.54592 | 0.012085 | 0.52832 | −0.9205 | 0.000821 |
| Placenta | RR | POS | TAG 50:6 | Triacylglycerol | 840.7072541 | 13.62613 | 0.68213 | −0.55188 | 0.023585 | 0.25922 | −1.9477 | 3.55E−05 |
| Placenta | RR | POS | MGDG 34:6 | Monogalactosyldiacylglycerol | 764.5291036 | 6.109162 | 0.6795 | −0.55746 | 0.036011 | 0.51217 | −0.96529 | 0.018498 |
| Placenta | RR | NEG | PI 36:6 | Phosphatidylinositol | 853.486525 | 5.136754 | 0.67162 | −0.57429 | 0.045268 | 0.54919 | −0.86462 | 0.037662 |
| Placenta | RR | POS | PC 36:6 | Phosphatidylcholine | 778.5370782 | 6.221897 | 0.67036 | −0.57699 | 0.036905 | 0.53767 | −0.8952 | 0.010129 |
| Placenta | RR | POS | PE 34:3 | Phosphatidylethanolamine | 714.5051574 | 7.433308 | 0.65861 | −0.60251 | 0.037689 | 0.52858 | −0.91982 | 0.000538 |
| Placenta | RR | NEG | SQDG 32:3 | Sulfoquinovosyl diacylglycerol | 787.4997841 | 5.329678 | 0.63177 | −0.66253 | 0.002571 | 0.49222 | −1.0226 | 0.000501 |
| Placenta | RR | POS | PE 36:6 | Phosphatidylethanolamine | 736.490567 | 6.333115 | 0.59924 | −0.7388 | 0.029383 | 0.49917 | −1.0024 | 0.000609 |
| Placenta | RR | POS | DGDG 34:3 | Digalactosyldiacylglycerol | 932.6299042 | 7.225584 | 0.59054 | −0.7599 | 0.008398 | 0.41062 | −1.2841 | 0.000325 |
| Placenta | RR | NEG | SQDG 34:3 | Sulfoquinovosyl diacylglycerol | 815.4974281 | 5.901407 | 0.57761 | −0.79184 | 0.00098 | 0.41742 | −1.2604 | 0.000501 |
| Placenta | RR | POS | DGDG 34:4 | Digalactosyldiacylglycerol | 930.6149915 | 6.528241 | 0.52571 | −0.92767 | 0.033065 | 0.22776 | −2.1344 | 0.000144 |
| Placenta | RR | NEG | SQDG 36:6 | Sulfoquinovosyl diacylglycerol | 837.4821293 | 5.145032 | 0.49774 | −1.0065 | 0.001265 | 0.43671 | −1.1953 | 0.000501 |
| Pericarp | 20 dpa | NEG | PG 36:5 | Phosphatidylglycerol | 767.487021 | 5.773432 | 0.791 | −0.33825 | 0.030182 | 0.68696 | −0.5417 | 0.002244 |
| Pericarp | 20 dpa | NEG | PS 42:4 | Phosphatidylserine | 866.59383 | 7.629547 | 0.7502 | −0.41465 | 0.018009 | 0.69238 | −0.53037 | 0.004317 |
| Pericarp | 20 dpa | NEG | PS 40:5 | Phosphatidylserine | 836.5445562 | 6.222667 | 0.67697 | −0.56283 | 0.037507 | 0.48637 | −1.0399 | 0.002657 |
| Pericarp | 20 dpa | POS | TAG 52:6 | Triacylglycerol | 868.7384924 | 14.10849 | 0.66714 | −0.58394 | 0.028419 | 0.59662 | −0.74511 | 0.012242 |
| Pericarp | 20 dpa | POS | TAG 54:8 | Triacylglycerol | 892.7383921 | 13.78206 | 0.64086 | −0.64191 | 0.038419 | 0.70259 | −0.50924 | 0.021277 |
| Pericarp | 20 dpa | POS | TAG 54:9 | Triacylglycerol | 890.7224362 | 13.35717 | 0.62559 | −0.6767 | 0.023275 | 0.60231 | −0.73142 | 0.020292 |
| Pericarp | 20 dpa | POS | PE 38:3 | Phosphatidylethanolamine | 770.5691763 | 7.776976 | 0.52149 | −0.93929 | 0.033275 | 0.60492 | −0.72518 | 0.033791 |
| Pericarp | MG | NEG | SQDG 32:3 | Sulfoquinovosyl diacylglycerol | 787.4997841 | 5.329678 | 0.68605 | −0.54361 | 0.089538 | 0.73008 | −0.45386 | 0.058076 |
| Pericarp | RR | NEG | SQDG 32:1 | Sulfoquinovosyl diacylglycerol | 791.5319008 | 6.259773 | 1.5718 | 0.65239 | 0.019293 | 1.6885 | 0.75578 | 0.01981 |
| Pericarp | RR | POS | PC 36:6 | Phosphatidylcholine | 778.5370782 | 6.221897 | 0.69895 | −0.51675 | 0.017319 | 0.53203 | −0.91043 | 0.000755 |
| Pericarp | RR | NEG | SQDG 34:3 | Sulfoquinovosyl diacylglycerol | 815.4974281 | 5.901407 | 0.69487 | −0.52518 | 0.01054 | 0.53538 | −0.90136 | 0.001056 |
| Pericarp | RR | POS | TAG 54:8 | Triacylglycerol | 892.7383921 | 13.78206 | 0.68071 | −0.55488 | 0.034655 | 0.41013 | −1.2859 | 0.000271 |
| Pericarp | RR | NEG | PI 36:6 | Phosphatidylinositol | 853.486525 | 5.136754 | 0.66842 | −0.58117 | 0.015658 | 0.7525 | −0.41024 | 0.02586 |
| Pericarp | RR | NEG | SQDG 36:6 | Sulfoquinovosyl diacylglycerol | 837.4821293 | 5.145032 | 0.62719 | −0.67302 | 0.038537 | 0.69063 | −0.53401 | 0.007133 |
| Pericarp | RR | NEG | PS 40:5 | Phosphatidylserine | 836.5445562 | 6.222667 | 0.62298 | −0.68274 | 0.017002 | 0.47077 | −1.0869 | 0.000694 |
| Pericarp | RR | POS | DGDG 34:4 | Digalactosyldiacylglycerol | 930.6149915 | 6.528241 | 0.5254 | −0.92851 | 0.016439 | 0.27487 | −1.8632 | 0.000313 |

TABLE 9
Gene identifiers for FIG. 4
Genes included in FIG. 4

| Gene ID | Annotation | Function |
|---|---|---|
| Solyc06g071580 | MORC family protein | Chromatin remodeling |
| Solyc12g042000 | BREAST CANCER ASSOCIATED RING 1 like protein | DNA repair |
| Solyc12g099910 | Chromodomain helicase DNA binding protein | Chromatin remodeling |
| Solyc01g087400 | Histone-lysine N-methyltransferase | Chromatin remodeling |
| Solyc11g062010 | Chromodomain helicase DNA binding protein | Chromatin remodeling |
| Solyc01g006880 | Histone-lysine N-methyltransferase | Chromatin remodeling |
| Solyc10g006200 | Endoribonuclease Dicer homolog 1 | Gene silencing/siRNA producion |
| Solyc02g091620 | DNA mismatch repair protein-like | DNA repair |
| Solyc09g066080 | BREAST CANCER ASSOCIATED RING 1 like protein | DNA repair |
| Solyc01g098520 | DNA mismatch repair protein-like | DNA repair |
| Solyc11g008530 | Dicer-like protein SlDCL2d | Gene silencing/siRNA producion |
| Solyc01g106770 | Serine/threonine-protein kinase TOR | TOR signaling |
| Solyc11g030550 | Cyclin B2 | Cell cycle |
| Solyc09g064200 | Myosin XI-2 | Cellular trafficking |
| Solyc02g090260 | Golgin | Cellular trafficking |
| Solyc10g076260 | Regulatory-associated protein of TOR RAPTOR1 | TOR signaling |
| Solyc10g076270 | Regulatory-associated protein of TOR RAPTOR2 | TOR signaling |
| Solyc05g013490 | Cyclin like protein | Cell cycle |
| Solyc08g075650 | Formin-like protein | Cellular trafficking |
| Solyc01g106950 | Myosin heavy chain-like protein | Cellular trafficking |
| Solyc02g069660 | Vacuolar protein sorting-associated protein 54 | Cellular trafficking |
| Solyc06g076560 | Heat shock protein Hsp20 | Stress responses |
| Solyc03g113930 | Heat shock protein Hsp20 | Stress responses |
| Solyc03g117630 | Heat shock protein 70 | Stress responses |
| Solyc12g011310 | Glutathione S-transferase | Stress responses |
| Solyc03g082420 | Heat shock protein Hsp20 | Stress responses |
| Solyc11g020330 | Leer-sHSP small heat shock protein | Stress responses |
| Solyc07g056510 | Glutathione S-transferase | Stress responses |
| Solyc02g093600 | Heat shock protein Hsp20 | Stress responses |
| Solyc02g072000 | Heat Stress transcription factor SolycHsfA4c | Transcription factor |
| Solyc01g100640 | Catalase | Stress responses |
| Solyc01g100630 | Catalase | Stress responses |
| Solyc06g033850 | DREB2D like transcription factor | Transcription factor |
| Solyc02g091030 | NF-YC4 like transcription factor | Transcription factor |
| Solyc03g080150 | Peroxidase | Stress responses |
| Solyc03g124110 | CBF2 like transcription factor | Transcription factor |
| Solyc10g050970 | Ethylene Response Factor SI-ERF.D4 | Transcription factor |
| Solyc08g007820 | CBF4/DDF2 like transcription factor | Transcription factor |
| Solyc08g007830 | CBF/DDF1 like transcription factor | Transcription factor |

TABLE 10
Gene identifiers for FIG. 7A
Genes included in FIG. 7A

| | | |
|---|---|---|
| Gene ID | Annotation | Function |

Placenta

| | | |
|---|---|---|
| Solyc02g036290 | Malonyl-CoA decarboxylase | Fatty acid metabolism |
| Solyc05g008790 | Ceramide kinase | Sphingolipid metabolism |
| Solyc02g082770 | Phosphatidylinositide phosphatase | Phosphatidylinositol dephosphorylation; Signaling |
| Solyc02g082763 | Phosphatidylinositide phosphatase | Phosphatidylinositol dephosphorylation; Signaling |
| Solyc09g075790 | Long-chain acyl-CoA synthetase (LACS) | Fatty acid elongation |
| Solyc07g032180 | Lysophosphatidic acid acyltransferase (LPAAT) | Glycerolipid biosynthesis; Phosphatidic acid |
| | | biosynthesis |
| Solyc05g042070 | Phosphatidylinositol-4-phosphate 5-kinase | Synthesis of phosphatidylinositol (4,5)-bisphosphate; |
| | | Lipid signaling |
| Solyc10g005840 | Farnesyl diphosphate synthase | Sterol biosynthesis |
| Solyc07g032170 | Lysophosphatidic acid acyltransferase | Glycerolipid biosynthesis, Phosphatidic acid |
| | | biosynthesis |
| Solyc07g032220 | Phospholipase A2 | Phospholipid metabolism; Cleavage at the sn-2 produce |
| | | lysophospholipid and free fatty acid |
| Solyc03g019670 | Phospholipase C | Phospholipid catabolism |
| Solyc02g084930 | Cytochrome P450 family protein | Sterol metabolism |
| Solyc08g080130 | Phospholipase PLDb1 | Phospholipid catabolismo Generates phosphatidic acid |
| | | (PA) and a free head group |
| Solyc11g011610 | Fatty acid-binding protein | Lipid biosynthesis |
| Solyc09g061840 | 3-Ketoacyl-CoA thiolase | Fatty acid β-oxidation |
| Solyc03g044910 | ELO family protein | Fatty acid elongation; Very-long-chain fatty acid |
| | | synthesis |
| Solyc07g056320 | Glycerol-3-phosphate acyltransferase (GPAT) | Glycerolipid biosynthesis |
| Solyc06g030590 | Subunit of serine palmitoyltransferase (LCB2) | Sphingolipid biosynthesis |
| Solyc10g086690 | Phosphatidylinositol:ceramide inositolphosphotransferase | Sphingolipid biosynthesis |
| | (IPCS1) | |
| Solyc12g100270 | Fatty acid hydroxylase | Sterol/Wax biosynthesis |
| Solyc04g040130 | Omega-6 fatty acid desaturase | Phospholipid desaturation in the ER; Synthesis of 18:2 |
| | | fatty acids in the endoplasmic reticulum |
| Solyc12g100250 | Omega-6 fatty acid desaturase | Phospholipid desaturation in the ER |
| Solyc02g038740 | 3-Hydroxy-3-methylglutaryl CoA reductase | Sterol biosynthesis |
| Solyc02g084740 | Cytochrome P450 family protein | Sterol metabolism |
| Solyc01g109140 | CYP74 cytochrome P450 family protein | Lipid oxidation/Oxylipin metabolism |
| Solyc04g079820 | Phosphatidylinositol polyphosphate 5-phosphatase | Phosphatidylinositol dephosphorylation; Signaling |
| Solyc08g029000 | Lipoxygenase | Lipid oxidation; Oxylipin biosynthesis |
| Solyc01g103650 | Phospholipase ABHD3 isoform | Phospholipid catabolism |
| Solyc01g005930 | Lipase/lipooxygenase, PLAT/LH2 family protein | Lipid oxidation; Oxylipin biosynthesis |
| Solyc01g095930 | O-acyltransferase WSD1-like protein | Wax ester and TGA biosynthesis |
| Solyc01g099180 | Lipoxygenase | Lipid oxidation; Oxylipin biosynthesis |
| Solyc01g099190 | Lipoxygenase B | Lipid oxidation; Oxylipin biosynthesis |
| Solyc03g115370 | Diacylglycerol kinase (DGK) | Phosphatidic acid production; Lipid signaling |
| Solyc07g054830 | Diacylglycerol kinase (DGK) | Phosphatidic acid production; Lipid signaling |

Pericarp

| | | |
|---|---|---|
| Solyc05g050090 | Fatty acid/sphingolipid desaturase | Sphingolipid metabolism |
| Solyc01g006000 | GPI mannosyltransferase 2 | Glycosylphosphatidylinositol-Anchor synthesis |
| Solyc07g032180 | Lysophosphatidic acid acyltransferase(LPAAT) | Glycerolipid biosynthesis; Phosphatidic acid |
| | | biosynthesis |
| Solyc01g080900 | CYP88A3 Cytochrome P450 family protein/ent-kaurenoic | Sterol biosynthesis |
| | acid oxidase | |
| Solyc01g103650 | Phospholipase ABHD3 isoform | Phospholipid catabolism |
| Solyc06g011530 | Hydroxymethylglutaryl-CoA lyase | Branched-chain amino acid catabolismproduction of |
| | | Acetyl-CoA |
| Solyc00g009110 | Phosphatidylinositol polyphosphate 5-phosphatase | Phosphatidylinositol dephosphorylation; Signaling |
| Solyc01g099180 | Lipoxygenase | Lipid oxidation; Oxilipin biosynthesis |
| Solyc01g099190 | Lipoxygenase B | Lipid oxidation; Oxylipin biosynthesis |
| Solyc06g053480 | Stearoyl-ACP desaturase | Omega-9 monounsaturated fatty acid biosynthesis |
| Solyc11g008680 | Stearoyl-ACP desaturase | Omega-9 monounsaturated fatty acid biosynthesis |
| Solyc01g099200 | Lipoxygenase | Lipid oxidation; Oxylipin biosynthesis |
| Solyc12g005380 | Diacylglycerol kinase | Phosphatidic acid production; Lipid signaling |
| Solyc08g005630 | Long-chain-alcohol oxidase | Wax biosynthesis |
| Solyc01g099170 | Lipoxygenase | Lipid oxidation; Oxylipin biosynthesis |

TABLE 11
Gene identifiers for FIG. 8
Genes included in FIG. 8

| Gene ID | Gene Name | Annotation | Function |
|---|---|---|---|
| Solyc04g049360 | ADCS | Aminodeoxychorismate synthase | Folic acid-containing compound biosynthetic process |
| Solyc03g115370 | DGK | Diacylglycerol kinase | Phosphatidic acid production; Lipid signaling |
| Solyc07g054830 | DGK | Diacylglycerol kinase | Phosphatidic acid production; Lipid signaling |
| Solyc03g044910 | ELO | Elongation of fatty acids protein | Fatty acid elongation; Very-long-chain fatty acid synthesis |
| Solyc04g009030 | GAPDH | Glyceraldehyde-3-phosphate dehydrogenase | Glycolysis |
| Solyc07g056320 | GPAT | Glycerol-3-phosphate acyltransferase | Glycerolipid biosynthesis |
| Solyc10g086690 | IPCS1 | Phosphatidylinositol:ceramide | Sphingolipid biosynthesis |
| | | inositolphosphotransferase | |
| Solyc09g075790 | LACS | Long-chain acyl-CoA synthetase | Fatty acid elongation |
| Solyc06g030590 | LCB2 | LONG-CHAIN BASE2 subunit of serine | Sphingolipid biosynthesis |
| | | palmitoyltransferase | |
| Solyc07g017670 | LKR | Lysine-ketoglutarate reductase | Amino acid metabolism |
| Solyc05g042070 | | PI kinase | Lipid signaling |
| Solyc02g082770 | | PI phosphatase | Lipid signaling |
| Solyc03g115820 | RPE | Ribulose-phosphate 3-epimerase | Pentose phosphate pathway |
| Solyc10g018300 | | Transketolase | Pentose phosphate pathway |

TABLE 12
Gene identifiers for FIG. 12
Genes included in FIG. 12

| Gene ID | Gene Name | Annotation | Function |
|---|---|---|---|
| Solyc01g095080 | ACS2 | 1-aminocyclopropane-1-carboxylic acid synthase-2 | Ethylene biosynthesis |
| Solyc05g050010 | ACS4 | 1-aminocyclopropane-1-carboxylic acid synthase-4 | Ethylene biosynthesis |
| Solyc02g036350 | ACO6 | 1-aminocyclopropane-1-carboxylate oxidase | Ethylene biosynthesis |
| Solyc06g053710 | ETR4 | Ethylene receptor homolog | Ethylene signaling |
| Solyc06g051800 | EXP1 | Expansin 1 | Cell wall modification |
| Solyc07g064190 | PME3 | Pectin methylesterase | Cell wall modification |
| Solyc08g005610 | XTH5 | Xyloglucan endotransglucosylase-hydrolase | Cell wall modification |
| Solyc10g080210 | PG | Polygalacturonase | Cell wall modification |

### Example 7—Characterization of asp1-CR13 KO Tomatos

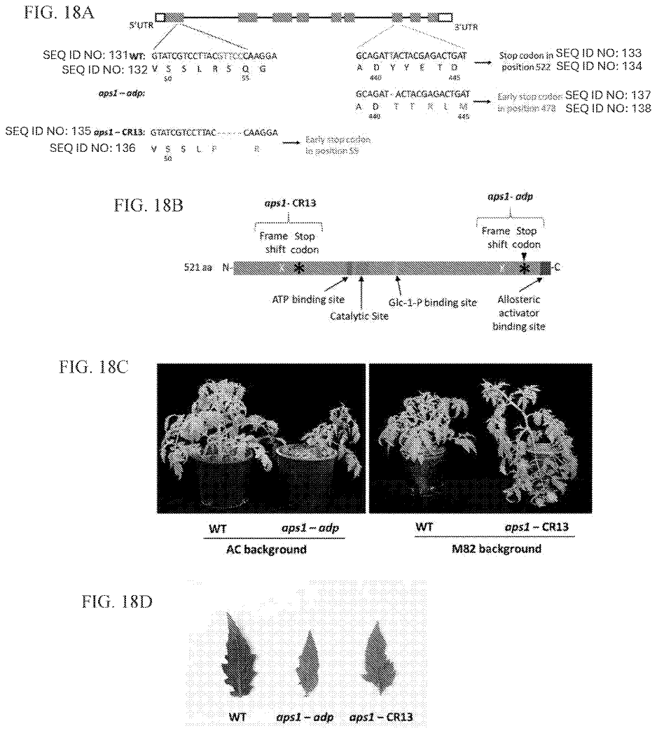

To test if another mutation/KO of APS1 would lead to a similar phenotype in another tomato cultivar, we generated CRISPR mutants targeting the APS1 gene in the tomato cv. M82 (FIG. 18A). Several independent CRISPR mutant lines were obtained by causing mutations in the different motifs and domains of the APS1 protein (FIG. 18B). We identified one homozygous line for the edited site (line CR13, harboring a 5 bp deletion at the target site leading to a frameshift and a very early stop codon in the APS1 gene (FIG. 18A).

At 6 weeks old, the aps1-CR13 KO tomato showed and altered vegaetative phenotype with decumbent stems caused by a lack of starch accumulation. Indeed the asp1-CR13 KO tomatoes showed a similar phenotype to aps1-adp (i.e. having an altered vegetative phenotype and no starch accumulation) (FIG. 18C). In stark contrast to the wild type plant, the leaves of both the asp1-adp and asp1-CR13 lacked any starch accumulation.

In view of this data, we have identified another mutation of the ASP1 protein that is capable of increasing blossom end rot resistance in, increasing the size of, and/or increasing the antioxidants in fruit of tomato plants.

### Example 8—ADP-Glucose Pyrophosphorylase Genes

Genomic sequences, coding sequences, and amino acid sequences for AGPase small subunits (APS) and large subunits (APL) from various Solanaceae and Curcubitae species are provided in Table 13. Tomato (Solanum lycopersicum Heinz ITAG 4.1), eggplant (Solanum melongena V3), and pepper (Capsicum annuum cv CM334 v1.55) sequences are from solgenomics.net. Cucumber (Cucumis sativus L. var. sativus cv. 9930 v3), squash (Cucurbita maxima Rimu), zucchini (Cucurbita pepo), melon (Cucumis melo DHL92 v3.6.1), watermelon (Citrullus lanatus 97103 v1), and pumpkin (Cucurbita moschata Rifu) sequences are from cucurbitgenomics.org.

Exemplary eggplant sequences include APS1 (Accession No. SMEL_007g292070), APS2 (Accession No. SMEL_008g302690.1.01), APL1 (Accession No. SMEL_001g137380.1), APL2 (Accession No. SMEL_006g246970.1.01), APL3 (Accession No. SMEL_001g117310.1.01), each of which is hereby incorporated by reference in its entirety).

Exemplary pepper sequences include APS7 (Accession No. CA07g15620), APS2 (Accession No. CA00g10930), APS3 (Accession No. CA09g08040), APL1 (Accession No. CA01g24580), APL2 (Accession No. CA07g05920), APL3 (Accession No. CA01g05120), each of which is hereby incorporated by reference in its entirety).

Exemplary cucumber sequences include APS1 (Accession No. CsaV3_7G002310), APL1 (Accession No. CsaV3_3G013570.1), APL2 (Accession No. CsaV3_3G045370.1), APL3 (Accession No. CsaV3_2G014910.1), each of which is hereby incorporated by reference in its entirety).

Exemplary squash sequences include APS1 (Accession No. CmaCh19G008330), APS3 (Accession No. CmaCh11G013000.1), APL1 (Accession No. CmaCh14G016860.1), APL2 (Accession No. CmaCh09G001450), APL3 (Accession No. CmaCh02G003870), each of which is hereby incorporated by reference in its entirety).

Exemplary zucchini sequences include APS1 (Accession No. Cp4.1LG15g06700), APL1 (Accession No. Cp4.1LG03g10860.1), APL2 (Accession No. Cp4.1LG06g01040.1), APL3 (Accession No. Cp4.1LG16g02590.1), each of which is hereby incorporated by reference in its entirety).

Exemplary melon sequences include APS1 (Accession No. MELO3C024175.2.1), APL1 (Accession No. MELO3C006552), APL2 (Accession No. MELO3C003812), APL3 (Accession No. MELO3C023310), each of which is hereby incorporated by reference in its entirety).

Exemplary watermelon sequences include APS1 (Accession No. Cla015710), APL1 APL1 (Accession No. Cla021651), APL2 (Accession No. Cla020783), APL3 (Accession No. Cla016574), each of which is hereby incorporated by reference in its entirety).

Exemplary pumpkin sequences include APS1 (Accession No. CmoCh19G008490), APS3 (Accession No. CmoCh11G018220), APL1 (Accession No. CmoCh14G017310), APL2 (Accession No. CmoCh09G001430), APL3 (Accession No. CmoCh02G003940), each of which is hereby incorporated by reference in its entirety).

TABLE 14
Exemplary APS and APL Sequences are set forth in the
Sequence Listing that is incorporated herein by reference.
Organism, Gene Name, Sequence Type (Accession No.) (SEQ ID NO:)

Solanum lycopersicum APS1 Genomic Sequence (Solyc07g056140) (SEQ ID NO: 1)
>Solyc07g056140_SL4.0ch07 SL4.0ch07: 63906315 . . . 63911061_APS1_tomato_genomic
Solanum lycopersicum APS1 Coding Sequence (Solyc07g056140) (SEQ ID NO: 2)
>Solyc07g056140.3.1_APS1 tomato_CDS
Solanum lycopersicum APS1 Amino Acid Sequence (Solyc07g056140) (SEQ ID NO: 3)
Solanum lycopersicum APS2 Genomic Sequence (Solyc08g015670) (SEQ ID NO: 4)
>Solyc08g015670_SL4.0ch08 SL4.0ch08: 5145629 . . . 5149158_APS2_tomato_genomic
Solanum lycopersicum APS2 Coding Sequence (Solyc08g015670) (SEQ ID NO: 5)
>Solyc08g015670.4.1_APS2_tomato_CDS
Solanum lycopersicum APS2 Amino Acid Sequence (Solyc08g015670) (SEQ ID NO: 6)
Solanum lycopersicum APS3 Genomic Sequence (Solyc12g011120) (SEQ ID NO: 7)
>Solyc12g011120_SL4.0ch12: 4007641 . . . 4012840_APS3_tomato_genomic
Solanum lycopersicum APS3 Coding Sequence (Solyc12g011120) (SEQ ID NO: 8)
>Solyc12g011120.4.1_APS3_tomato_CDS
Solanum lycopersicum APS3 Amino Acid Sequence (Solyc12g011120) (SEQ ID NO: 9)
Solanum lycopersicum APL1 Genomic Sequence (Solyc01g079790) (SEQ ID NO: 10)
>Solyc01g079790.2 SL2.50ch01: 78922547 . . . 78926789_APL1_tomato_genomic
Solanum lycopersicum APL1 Coding Sequence (Solyc01g079790) (SEQ ID NO: 11)
>Solyc01g079790.5.1_APL1 tomato_CDS
Solanum lycopersicum APL1 Amino Acid Sequence (Solyc01g079790) (SEQ ID NO: 12)
Solanum lycopersicum APL2 Genomic Sequence (Solyc07g019440) (SEQ ID NO: 13)
>Solyc07g019440.2 SL2.50ch07: 11198353 . . . 11193230_APL2_tomato_genomic
Solanum lycopersicum APL2 Coding Sequence (Solyc07g019440) (SEQ ID NO: 14)
>Solyc07g019440.3.1_APL2 tomato_CDS
Solanum lycopersicum APL2 Amino Acid Sequence (Solyc07g019440) (SEQ ID NO: 15)
Solanum lycopersicum APL3 Genomic Sequence (Solyc01g109790) (SEQ ID NO: 16)
>Solyc01g109790.2_SL2.50ch01: 96641133 . . . 96636500_APL3_tomato_genomic
Solanum lycopersicum APL3 Coding Sequence (Solyc01g109790) (SEQ ID NO: 17)
>Solyc01g109790.3.1_APL3 tomato_CDS
Solanum lycopersicum APL3 Amino Acid Sequence (Solyc01g109790) (SEQ ID NO: 18)
Solanum melongena APS1 Genomic Sequence (SMEL_007g292070) (SEQ ID NO: 19)
>SMEL_007g292070_SMEL3Ch07 SMEL3Ch07: 136735949 . . . 136741018_APS1_eggplant_genomic
Solanum melongena APS1 Coding Sequence (SMEL_007g292070) (SEQ ID NO: 20)
>SMEL_007g292070_SMEL_007g292070.1.01_APS1_eggplant_CDS
Solanum melongena APS1 Amino Acid Sequence (SMEL_007g292070) (SEQ ID NO: 21)
Solanum melongena APS2 Genomic Sequence (SMEL_008g302690) (SEQ ID NO: 22)
>SMEL_008g302690_SMEL3Ch08: 12017224 . . . 12020376_APS2_eggplant_genomic
Solanum melongena APS2 Coding Sequence (SMEL_008g302690) (SEQ ID NO: 23)
>SMEL_008g302690.1.01_APS2_eggplant_CDS
Solanum melongena APS2 Amino Acid Sequence (SMEL_008g302690) (SEQ ID NO: 24)
Solanum melongena APL1 Genomic Sequence (SMEL_001g137380) (SEQ ID NO: 25)
>SMEL_001g137380.1_SMEL3Ch01 SMEL3Ch01: 30484136 . . . 30490159 APL1_eggplant_genomic
Solanum melongena APL1 Coding Sequence (SMEL_001g137380) (SEQ ID NO: 26)
>SMEL_001g137380.1.01 SMEL_001g137380.1_APL1_eggplant_CDS
Solanum melongena APL1 Amino Acid Sequence (SMEL_001g137380) (SEQ ID NO: 27)
Solanum melongena APL2 Genomic Sequence (SMEL_006g246970) (SEQ ID NO: 28)
>SMEL_006g246970.1_SMEL3Ch06: 35481639 . . . 35487138_APL2_eggplant_genomic
Solanum melongena APL2 Coding Sequence (SMEL_006g246970) (SEQ ID NO: 29)
>SMEL_006g246970.1.01_SMEL_006g246970.1_APL2_eggplant_CDS
Solanum melongena APL2 Amino Acid Sequence (SMEL_006g246970) (SEQ ID NO: 30)
Solanum melongena APL3 Genomic Sequence (SMEL_001g117310) (SEQ ID NO: 31)
>SMEL_001g117310.1_SMEL3Ch01: 2170347 . . . 2175222_APL3_eggplant_genomic
Solanum melongena APL3 Coding Sequence (SMEL_001g117310) (SEQ ID NO: 32)
>SMEL_001g117310.1.01_SMEL_001g117310.1_APL3_eggplant_CDS
Solanum melongena APL3 Amino Acid Sequence (SMEL_001g117310) (SEQ ID NO: 33)
Capsicum annuum APS1 Genomic Sequence (CA07g15620) (SEQ ID NO: 34)
>CA07g15620_Pepper1.55ch07: 217965311 . . . 217970340_APS1_pepper_genomic
Capsicum annuum APS1 Coding Sequence (CA07g15620) (SEQ ID NO: 35)
>CA07g15620_APS1_pepper_CDS
Capsicum annuum APS1 Amino Acid Sequence (CA07g15620) (SEQ ID NO: 36)
Capsicum annuum APS2 Genomic Sequence (CA00g10930) (SEQ ID NO: 37)
>CA00g10930_PGAv.1.5.contig154378: 4700-6300_APS2_pepper_genomic
Capsicum annuum APS2 Coding Sequence (CA00g10930) (SEQ ID NO: 38)
>CA00g10930_APS2_pepper_CDS
Capsicum annuum APS2 Amino Acid Sequence (CA00g10930) (SEQ ID NO: 39)
Capsicum annuum APS3 Genomic Sequence (CA09g08040) (SEQ ID NO: 40)
>CA09g08040_Pepper1.55ch09: 72144554 . . . 72153286_APS3_pepper_genomic
Capsicum annuum APS3 Coding Sequence (CA09g08040) (SEQ ID NO: 41)
>CA09g08040_APS3_pepper_CDS
Capsicum annuum APS3 Amino Acid Sequence (CA09g08040) (SEQ ID NO: 42)
Capsicum annuum APL1 Genomic Sequence (CA01g24580) (SEQ ID NO: 43)
>CA01g24580_Pepper1.55ch01: 194778153 . . . 194782547_APL1_pepper_genomic
Capsicum annuum APL1 Coding Sequence (CA01g24580) (SEQ ID NO: 44)
>CA01g24580_APL1_pepper_CDS
Capsicum annuum APL1 Amino Acid Sequence (CA01g24580) (SEQ ID NO: 45)
Capsicum annuum APL2 Genomic Sequence (CA07g05920) (SEQ ID NO: 46)
>CA07g05920_Pepper1.55ch07: 58609942 . . . 58614480_APL2_pepper_genomic
Capsicum annuum APL2 Coding Sequence (CA07g05920) (SEQ ID NO: 47)
>CA07g05920_APL2_pepper_CDS
Capsicum annuum APL2 Amino Acid Sequence (CA07g05920) (SEQ ID NO: 48)
Capsicum annuum APL3 Genomic Sequence (CA01g05120) (SEQ ID NO: 49)
>CA01g05120_Pepper1.55ch01: 9269020 . . . 9272451_APL3_pepper_genomic
Capsicum annuum APL3 Coding Sequence (CA01g05120) (SEQ ID NO: 50)
>CA01g05120_APL3_pepper_CDS
Capsicum annuum APL3 Amino Acid Sequence (CA01g05120) (SEQ ID NO: 51)
Cucumis sativus APS1 Genomic Sequence (CsaV3_7G002310) (SEQ ID NO: 52)
>CsaV3_7G002310_chr7: 1820139 . . . 1826014_APS1_cucumber_genomic
Cucumis sativus APS1 Coding Sequence (CsaV3_7G002310) (SEQ ID NO: 53)
>CsaV3_7G002310_APS1_cucumber_CDS
Cucumis sativus APS1 Amino Acid Sequence (CsaV3_7G002310) (SEQ ID NO: 54)
Cucumis sativus APL1 Genomic Sequence (CsaV3_3G013570) (SEQ ID NO: 55)
>CsaV3_3G013570_chr3: 10239897 . . . 10244727_APL1_cucumber_genomic
Cucumis sativus APL1 Coding Sequence (CsaV3_3G013570) (SEQ ID NO: 56)
>CsaV3_3G013570.1_APL1_cucumber_CDS
Cucumis sativus APL1 Amino Acid Sequence (CsaV3_3G013570) (SEQ ID NO: 57)
Cucumis sativus APL2 Genomic Sequence (CsaV3_3G045370) (SEQ ID NO: 58)
>CsaV3_3G045370_chr3: 37045768 . . . 37050213_APL2_cucumber_genomic
Cucumis sativus APL2 Coding Sequence (CsaV3_3G045370) (SEQ ID NO: 59)
>CsaV3_3G045370.1_APL2_cucumber_CDS
Cucumis sativus APL2 Amino Acid Sequence (CsaV3_3G045370) (SEQ ID NO: 60)
Cucumis sativus APL3 Genomic Sequence (CsaV3_2G014910) (SEQ ID NO: 61)
>CsaV3_2G014910_chr2: 12410201 . . . 12415462_APL3_cucumber_genomic
Cucumis sativus APL3 Coding Sequence (CsaV3_2G014910) (SEQ ID NO: 62)
>CsaV3_2G014910.1_APL3_cucumber_CDS
Cucumis sativus APL3 Amino Acid Sequence (CsaV3_2G014910) (SEQ ID NO: 63)
Cucurbita maxima APS1 Genomic Sequence (CmaCh19G008330) (SEQ ID NO: 64)
>CmaCh19G008330_APS1_squash_genomic
Cucurbita maxima APS1 Coding Sequence (CmaCh19G008330) (SEQ ID NO: 65)
>CmaCh19G008330_APS1_squash_CDS
Cucurbita maxima APS1 Amino Acid Sequence (CmaCh19G008330) (SEQ ID NO: 66)
Cucurbita maxima APS3 Genomic Sequence (CmaCh11G013000) (SEQ ID NO: 67)
>CmaCh11G013000_APS3_squash_genomic
Cucurbita maxima APS3 Coding Sequence (CmaCh11G013000) (SEQ ID NO: 68)
>CmaCh11G013000.1_APS3_squash_CDS
Cucurbita maxima APS3 Amino Acid Sequence (CmaCh11G013000) (SEQ ID NO: 69)
Cucurbita maxima APL1 Genomic Sequence (CmaCh14G016860) (SEQ ID NO: 70)
>CmaCh14G016860_APL1_squash_genomic
Cucurbita maxima APL1 Coding Sequence (CmaCh14G016860) (SEQ ID NO: 71)
>CmaCh14G016860.1_APL1_squash_CDS
Cucurbita maxima APL1 Amino Acid Sequence (CmaCh14G016860) (SEQ ID NO: 72)
Cucurbita maxima APL2 Genomic Sequence (CmaCh09G001450) (SEQ ID NO: 73)
>CmaCh09G001450_APL2_squash_genomic
Cucurbita maxima APL2 Coding Sequence (CmaCh09G001450) (SEQ ID NO: 74)
>CmaCh09G001450_APL2_squash_CDS
Cucurbita maxima APL2 Amino Acid Sequence (CmaCh09G001450) (SEQ ID NO: 75)
Cucurbita maxima APL3 Genomic Sequence (CmaCh02G003870) (SEQ ID NO: 76)
>CmaCh02G003870_APL3_squash_genomic
Cucurbita maxima APL3 Coding Sequence (CmaCh02G003870) (SEQ ID NO: 77)
>CmaCh02G003870_APL3_squash_CDS
Cucurbita maxima APL3 Amino Acid Sequence (CmaCh02G003870) (SEQ ID NO: 78)
Cucurbita pepo APS1 Genomic Sequence (Cp4.1LG15g06700) (SEQ ID NO: 79)
>Cp4.1LG15g06700_APS1_zucchini_genomic
Cucurbita pepo APS1 Coding Sequence (Cp4.1LG15g06700) (SEQ ID NO: 80)
>Cp4.1LG15g06700_APS1_zucchini_CDS
Cucurbita pepo APS1 Amino Acid Sequence (Cp4.1LG15g06700) (SEQ ID NO: 81)
Cucurbita pepo APL1 Genomic Sequence (Cp4.1LG03g10860) (SEQ ID NO: 82)
>Cp4.1LG03g10860_Cp4.1LG03: 11416566 . . . 11424647_APL1_zucchini_genomic
Cucurbita pepo APL1 Coding Sequence (Cp4.1LG03g10860) (SEQ ID NO: 83)
>Cp4.1LG03g10860.1_APL1_zucchini_CDS
Cucurbita pepo APL1 Amino Acid Sequence (Cp4.1LG03g10860) (SEQ ID NO: 84)
Cucurbita pepo APL2 Genomic Sequence (Cp4.1LG06g01040) (SEQ ID NO: 85)
>Cp4.1LG06g01040_Cp4.1LG06: 497971 . . . 502927_APL2_zucchini_genomic
Cucurbita pepo APL2 Coding Sequence (Cp4.1LG06g01040) (SEQ ID NO: 86)
>Cp4.1LG06g01040.1_APL2_zucchini_CDS
Cucurbita pepo APL2 Amino Acid Sequence (Cp4.1LG15g06700) (SEQ ID NO: 87)
Cucurbita pepo APL3 Genomic Sequence (Cp4.1LG16g02590) (SEQ ID NO: 88)
>Cp4.1LG16g02590_Cp4.1LG16: 4527849 . . . 4532781_APL3_zucchini_genomic
Cucurbita pepo APL3 Coding Sequence (Cp4.1LG16g02590) (SEQ ID NO: 89)
>Cp4.1LG16g02590.1_APL3_zucchini_CDS
Cucurbita pepo APL3 Amino Acid Sequence (Cp4.1LG16g02590) (SEQ ID NO: 90)
Cucumis melo APS1 Genomic Sequence (MELO3C024175) (SEQ ID NO: 91)
>MELO3C024175.2.1_APS1_melon_genomic
Cucumis melo APS1 Coding Sequence (MELO3C024175) (SEQ ID NO: 92)
>MELO3C024175.2.1_APS1_melon_CDS
Cucumis melo APS1 Amino Acid Sequence (MELO3C024175) (SEQ ID NO: 93)
Cucumis melo APL1 Genomic Sequence (MELO3C006552) (SEQ ID NO: 94)
>MELO3C006552_APL1_melon_genomic
Cucumis melo APL1 Coding Sequence (MELO3C006552) (SEQ ID NO: 95)
>MELO3C006552_APL1_melon_CDS
Cucumis melo APL1 Amino Acid Sequence (MELO3C006552) (SEQ ID NO: 96)
Cucumis melo APL2 Genomic Sequence (MELO3C003812) (SEQ ID NO: 97)
>MELO3C003812_APL2_melon_genomic
Cucumis melo APL2 Coding Sequence (MELO3C003812) (SEQ ID NO: 98)
>MELO3C003812_APL2_melon_CDS
Cucumis melo APL2 Amino Acid Sequence (MELO3C003812) (SEQ ID NO: 99)
Cucumis melo APL3 Genomic Sequence (MELO3C023310) (SEQ ID NO: 100)
>MELO3C023310_APL3_melon_genomic
Cucumis melo APL3 Coding Sequence (MELO3C023310) (SEQ ID NO: 101)
>MELO3C023310_APL3_melon_CDS
Cucumis melo APL3 Amino Acid Sequence (MELO3C023310) (SEQ ID NO: 102)
Citrullus lanatus APS1 Genomic Sequence (Cla015710) (SEQ ID NO: 103)
>Cla015710_APS1_watermelon_genomic
Citrullus lanatus APS1 Coding Sequence (Cla015710) (SEQ ID NO: 104)
>Cla015710_APS1_watermelon_CDS
Citrullus lanatus APS1 Amino Acid Sequence (Cla015710) (SEQ ID NO: 105)
Citrullus lanatus APL1 Genomic Sequence (Cla021651) (SEQ ID NO: 106)
>Cla021651_APL1_watermelon_genomic
Citrullus lanatus APL1 Coding Sequence (Cla021651) (SEQ ID NO: 107)
>Cla021651_APL1_watermelon_CDS
Citrullus lanatus APS1 Amino Acid Sequence (Cla021651) (SEQ ID NO: 108)
Citrullus lanatus APL2 Genomic Sequence (Cla020783) (SEQ ID NO: 109)
>Cla020783_APL2_watermelon_genomic
Citrullus lanatus APL2 Coding Sequence (Cla020783) (SEQ ID NO: 110)
>Cla020783_APL2_watermelon_CDS
Citrullus lanatus APL2 Amino Acid Sequence (Cla015710) (SEQ ID NO: 111)
Citrullus lanatus APL3 Genomic Sequence (Cla020783) (SEQ ID NO: 112)
>Cla016574_APL3_watermelon_genomic
Citrullus lanatus APL3 Coding Sequence (Cla020783) (SEQ ID NO: 113)
>Cla016574_APL3_watermelon_CDS
Citrullus lanatus APL3 Amino Acid Sequence (Cla020783) (SEQ ID NO: 114)
Cucurbita moschata APS1 Genomic Sequence (CmoCh19G008490) (SEQ ID NO: 115)
>CmoCh19G008490APS1_pumpkin_genomic
Cucurbita moschata APS1 Coding Sequence (CmoCh19G008490) (SEQ ID NO: 116)
>CmoCh19G008490_APS1_pumpkin_CDS
Cucurbita moschata APS1 Amino Acid Sequence (CmoCh19G008490) (SEQ ID NO: 117)
Cucurbita moschata APS3 Genomic Sequence (CmoCh11G018220) (SEQ ID NO: 118)
>CmoCh11G018220_APS3_pumpkin_genomic
Cucurbita moschata APS3 Coding Sequence (CmoCh11G018220) (SEQ ID NO: 119)
>CmoCh11G018220_APS3_pumpkin_CDS
Cucurbita moschata APS3 Genomic Sequence (CmoCh11G018220) (SEQ ID NO: 120)
Cucurbita moschata APL1 Genomic Sequence (CmoCh14G017310) (SEQ ID NO: 121)
>CmoCh14G017310_APL1_pumpkin_genomic
Cucurbita moschata APL1 Coding Sequence (CmoCh14G017310) (SEQ ID NO: 122)
>CmoCh14G017310_APL1_pumpkin_CDS
Cucurbita moschata APL1 Amino Acid Sequence (CmoCh14G017310) (SEQ ID NO: 123)
Cucurbita moschata APL2 Genomic Sequence (CmoCh09G001430) (SEQ ID NO: 124)
>CmoCh09G001430_APL2_pumpkin_genomic
Cucurbita moschata APL2 Coding Sequence (CmoCh09G001430) (SEQ ID NO: 125)
>CmoCh09G001430_APL2_pumpkin_CDS
Cucurbita moschata APL2 Amino Acid Sequence (CmoCh09G001430) (SEQ ID NO: 126)
Cucurbita moschata APL3 Genomic Sequence (CmoCh02G003940) (SEQ ID NO: 127)
>CmoCh02G003940_APL3_pumpkin_genomic
Cucurbita moschata APL3 Coding Sequence (CmoCh02G003940) (SEQ ID NO: 128)
>CmoCh02G003940_APL3_pumpkin_CDS
Cucurbita moschata APL3 Amino Acid Sequence (CmoCh02G003940) (SEQ ID NO: 129)

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A plant cell comprising:
a heterologous nucleic acid molecule having
a) a modified ADP-glucose pyrophosphorylase (AGPase) sequence encoding an altered AGPase protein subunit or fragment thereof, or
b) an inhibitory polynucleotide sequence targeting an AGPase gene encoding an AGPase protein,
wherein the expression, stability, and/or activity of the AGPase protein is reduced or eliminated in the plant cell as compared to a plant cell without the modification.

2. The plant cell of claim 1, wherein the nucleic acid molecule comprises a modified ADP-glucose pyrophosphorylase (AGPase) sequence encoding an altered AGPase protein subunit or fragment thereof.

3. The plant cell according to claim 1 or 2, wherein said heterologous nucleic acid is a modified ADP-glucose pyrophosphorylase small subunit (APS) polynucleotide encoding an amino acid sequence that is at least 90% identical to any one of SEQ ID NOs: 3, 6, 9, 21, 24, 36, 39, 42, 54, 66, 69, 81, 93, 105, 117, and 120.

4. The plant cell according to claim 3, wherein the amino acid sequence is at least 90% identical to SEQ ID NO: 3, 6, or 9.

5. The plant cell according to claim 3, wherein the amino acid sequence is at least 90% identical to SEQ ID NO: 54.

6. The plant cell according to any one of claims 1-5, wherein the modified APS polynucleotide is SEQ ID NO: 130 comprising a deletion causing a frameshift mutation.

7. The plant cell according to claim 1 or 2, wherein said heterologous nucleic acid is a modified ADP-glucose pyrophosphorylase large subunit (APL) polynucleotide encoding an amino acid sequence that is at least 90% identical to any one of SEQ ID NOs: 12, 15, 18, 27, 30, 33, 45, 48, 51, 57, 60, 63, 72, 75, 78, 84, 87, 90, 96, 99, 102, 108, 111, 114, 123, 126, and 129.

8. The plant cell according to claim 7, wherein the amino acid sequence is at least 90% identical to SEQ ID NO: 12, 15, or 18.

9. The plant cell according to claim 7, wherein the amino acid sequence is at least 90% identical to SEQ ID NO: 57, 60, or 63.

10. The plant cell according to any one of claims 1-9, further comprising a second nucleic acid molecule encoding a functional AGPase protein operably linked to a promoter which functions in a non-fruit tissue of the plant.

11. The plant cell of claim 10, wherein the tissue specific promoter is selected from the promoters listed in table 14.

12. A plant comprising the plant cell of any one of claims 1-11.

13. The plant or plant cell according to claim 12, which is a member of the Cucurbitaceae family.

14. The plant or plant cell according to claim 13, wherein the plant or plant cell is selected from the genus Cucurbita, Cucumis, or Citrullus.

15. The plant or plant cell according to claim 13, wherein the plant or plant cell is selected from a zucchini, cucumber, watermelon, pumpkin, squash, or melon.

16. The plant or plant cell according to claim 12, which is a member of the Solanaccac family.

17. The plant or plant cell according to claim 16, wherein the plant or plant cell is from the genus Solanum or Capsicum.

18. The plant or plant cell according to claim 17, wherein the plant or plant cell is an eggplant or pepper.

19. The plant or plant cell according to claim 17, wherein the plant or plant cell is selected from a tomato plant or tomato plant cell.

20. The tomato plant or plant cell according to claim 19, selected from Alicante, Amish Paste, Aunt Ruby's German Green, Azoychka, Beefsteak, Berkeley Tie-Dye Green, Better Boy, Big Beef, Big Mama, Big Rainbow, Blaby Special, Black Beauty, Black Cherry, Black Icicle, Black Krim, Brandywine, Breonice, Campari, Canario, Carbon, Celebrity, Cherokee Purple, Cherry Bambelo, Cherry Nebula, Chocolate Pear, Dad's Sunset, Dester, Dr. Wyche's Yellow, Early Girl, Ed's Millennium, Emerald Evergreen, Enchantment, Ferreira, Ferris Wheel, Flamenco, Fourth of July, Garden Peach, Gardener's Delight, German Johnson, German Lunchbox, German Pink, Giulietta F1, Granadero, Great White, Green Doctors, Green Giant, Green Zebra, Hanover tomato, Henderson's Pink Ponderosa, Heinz, Hillbilly, Hungarian Heart, Japanese Black Trifele, Jersey Boy, Jubilee, Juliet, Kellogg's Breakfast, Kentucky Beefsteak, Kumato, Lillian's Yellow, Malakhitovaya Shkatulka, Matt's Wild Cherry, McDreamy, Micro Tom, Millionaire, Moneymaker, Monterosa, Montserrat, Mortgage Lifter, Mr. Stripey, Mushroom Basket, M82, Napa Rose Blush, Orange Hat, Orange Icicle, Pantano Romanesco, Paul Robeson, Pink Boar, Plum tomato, Raf tomato, Raspberry Lyanna, Rebekah Allen, Rebellion, Red Currant, Roma, Rosa de Barbastro, Rosella, Rutgers, San Marzano, Santorini, Sasha Altai, Scorpio (Skorpion), Stupice, Super Sweet 100, Thorburn's Terra-cotta, Tigerella, Tiny Tim, Tomaccio, Tomkin, Traveller/Arkansas Traveler, True Black Brandywine, Violet Jasper (Tzi Bi U), Viva Italia, Wagner Blue Green, White Queen, Yellow Brandywine, Yellow Pear, or hybrids thereof.

21. A fruit of the plant according to any one of claims 12-20.

22. A method of enhancing at least one of blossom end rot resistance to, size of, or antioxidants in fruit from a plant comprising:
a) introducing, into a plant cell, an exogenous nucleic acid molecule comprising
i) a modified ADP-glucose pyrophosphorylase (AGPase) sequence encoding an altered AGPase subunit protein or fragment thereof, or
ii) an inhibitory polynucleotide sequence targeting an AGPase gene encoding an AGPase protein, and
b) regenerating a transgenic plant from the plant cell.

23. The method of claim 22, wherein the nucleic acid molecule comprises a modified ADP-glucose pyrophosphorylase (AGPase) sequence encoding an altered AGPase subunit protein or fragment thereof.

24. The method of claim 22 or 23, wherein said reduction or elimination of the expression, stability, and/or activity of AGPase protein is effective in reducing or eliminating starch synthesis and
a) imparting blossom end rot resistance to fruit from the plant or a plant produced from the plant cell, as compared to fruit from a plant without the modification, and/or
b) increasing fruit size in fruit from the plant or a plant produced from the plant cell, as compared to fruit from a plant without the modification, and/or
c) increasing antioxidant levels in a fruit from the plant or a plant produced from the plant cell, as compared to a fruit from a plant without the modification.

25. The method of claim 24, wherein the increased fruit size comprises an increase in fruit weight by at least 10%.

26. The method of claim 24, wherein the increased fruit size comprises an increase in fruit volume by at least 5%.

27. The method of claim 24, wherein the increased antioxidants comprise raffinose, phenolics, and/or pyridoxal.

28. The method according to claim 22 or 23, wherein said heterologous nucleic acid is a modified ADP-glucose pyrophosphorylase small subunit (APS) polynucleotide encoding an amino acid sequence that is at least 90% identical to any one of SEQ ID NOs: 3, 6, 9, 21, 24, 36, 39, 42, 54, 66, 69, 81, 93, 105, 117, and 120.

29. The method of claim 28, wherein the amino acid sequence is at least 90% identical to SEQ ID NO: 3, 6, or 9.

30. The method according to claim 28, wherein the amino acid sequence is at least 90% identical to SEQ ID NO: 54.

31. The method according to any one of claims 22-30, wherein the modified APS polynucleotide is SEQ ID NO: 130 and comprises a deletion mutation causing a frameshift mutation.

32. The method according to claim 22 or 23, wherein said heterologous nucleic acid is a modified ADP-glucose pyrophosphorylase large subunit (APL) polynucleotide encoding an amino acid sequence that is at least 90% identical to any one of SEQ ID NOs: 12, 15, 18, 27, 30, 33, 45, 48, 51, 57, 60, 63, 72, 75, 78, 84, 87, 90, 96, 99, 102, 108, 111, 114, 123, 126, and 129.

33. The method according to claim 32, wherein the amino acid sequence is at least 90% identical to SEQ ID NO: 12, 15, or 18.

34. The method according to claim 32, wherein the amino acid sequence is at least 90% identical to SEQ ID NO: 57, 60, or 63.

35. The method according to anyone of claims 22-34, wherein the modified AGPase gene comprises a mutation in a coding sequence and/or regulatory sequence.

36. The method of claim 35, wherein the mutation is an insertion, a deletion, a substitution mutation, or any combination thereof.

37. The method according to claim 22, further comprising introducing a second nucleic acid molecule encoding a functional AGPase protein operably linked to a promoter which functions in a non-fruit tissue of the plant.

38. The method of claim 37, wherein the tissue specific promoter is selected from the promoters listed in table 14.

39. A plant or plant cell produced from the method of any one of claims 22-38.

40. A nucleic acid construct comprising:
a nucleotide sequence targeting an AGPase encoding nucleic acid comprising:
(i) a guide RNA; or
(ii) an inhibitory polynucleotide;
a 5′ heterologous DNA promoter sequence; and
a 3′ terminator sequence.

41. An expression vector comprising the nucleic acid construct according to claim 40.

42. The nucleic acid construct of claim 40 or the expression vector of claim 41, further comprising a genome editing nuclease.

43. The nucleic acid construct of claim 40 or the expression vector of claim 41, targeting a sequence present in a small subunit of said AGPase gene (APS) and inhibiting expression of a protein having an amino acid sequence selected from any one of SEQ ID NOs: 3, 6, 9, 21, 24, 36, 39, 42, 54, 66, 69, 81, 93, 105, 117, and 120, or having at least 90% sequence identity to any one of SEQ ID NOs: 3, 6, 9, 21, 24, 36, 39, 42, 54, 66, 69, 81, 93, 105, 117, and 120.

44. The nucleic acid construct or the expression vector of claim 43, for inhibition of an amino acid sequence at least 90% identical to SEQ ID NO: 3, 6, or 9.

45. The nucleic acid construct or the expression vector of claim 43, for inhibition of expression of an amino acid sequence at least 90% identical to SEQ ID NO: 54.

46. The nucleic acid construct or the expression vector of claim 43, wherein the modified APS polynucleotide is SEQ ID NO: 130 and comprises a deletion causing a frameshift mutation.

47. The nucleic acid construct of claim 40 or the expression vector of claim 41, targeting a sequence present in a small subunit of said AGPase gene (APL) and inhibiting expression of an amino acid sequence selected from of any one of SEQ ID NOs: 12, 15, 18, 27, 30, 33, 45, 48, 51, 57, 60, 63, 72, 75, 78, 84, 87, 90, 96, 99, 102, 108, 111, 114, 123, 126, and 129 or having at least 90% sequence identity to any one of SEQ ID NOs: 12, 15, 18, 27, 30, 33, 45, 48, 51, 57, 60, 63, 72, 75, 78, 84, 87, 90, 96, 99, 102, 108, 111, 114, 123, 126, and 129.

48. The nucleic acid construct or the expression vector of claim 47, for inhibiting expression of an amino acid sequence at least 90% identical to SEQ ID NO: 12, 15, or 18.

49. The nucleic acid construct or the expression vector of claim 47, for inhibiting expression of an amino acid sequence at least 90% identical to SEQ ID NO: 57, 60, or 63.

50. The nucleic acid construct of claim 40 or the expression vector of claim 41, further comprising a second nucleic acid molecule encoding a functional AGPase protein operably linked to a promoter which functions in a non-fruit tissue of the plant.

51. A cell transformed with the nucleic acid construct according to any one of claims 40-50.

52. The cell according to claim 51, wherein the cell is a bacterial cell or a plant cell.

53. A plant transformed with the nucleic acid construct according to any one of claims 40-50.

54. The cell or plant according to claim 52 or 53, wherein the plant cell or plant is from the Solanaceae or Cucurbitaceae family.

55. The cell or plant according to any one of claims 51-54, selected from a tomato, eggplant, pepper, zucchini, cucumber, watermelon, pumpkin, squash, or melon.

56. The cell or plant according to any one of claims 51-55, selected from the genus Solanum, Capsicum, Cucurbita, Cucumis, or Citrullus.

57. The cell or plant according to any one of claims 51-55, which is a tomato plant or tomato plant cell.

58. A fruit from the plant, or plant grown from the plant cell, according to any one of claims 51-57.

59. A plant seed produced from the plant according to any one of claims 51-58.

60. A method for breeding plants having enhanced blossom end rot resistance, said method comprising:
providing a candidate plant or plant part;
analyzing the candidate plant or plant part for the presence, in its genome, of a modified ADP-glucose pyrophosphorylase (AGPase) polynucleotide encoding an AGPase subunit protein, wherein the expression, stability, and/or activity of said AGPase subunit protein is reduced or eliminated as compared to a plant without the modification;
identifying, based on said analyzing, a candidate plant suitable for breeding that includes in its genome, the modified AGPase polynucleotide; and
breeding the identified plant with at least one other plant.

61. The method according to claim 60, wherein said analyzing comprises:
isolating nucleic acids from the plant or plant part;
analyzing nucleic acids from the plant or plant part for the presence of the modified AGPase polynucleotide; and
detecting the modified AGPase polynucleotide.

62. The method according to claim 60, wherein said breeding comprises crossing, making hybrids, backcrossing, self-crossing, double haploid breeding, and/or combinations thereof.

63. The method according to any one of claims 60-62, wherein said AGPase comprises a modified ADP-glucose pyrophosphorylase small subunit (APS) polynucleotide encoding an amino acid sequence that is at least 90% identical to any one of SEQ ID NOs: 3, 6, 9, 21, 24, 36, 39, 42, 54, 66, 69, 81, 93, 105, 117, and 120.

64. The method of claim 63, wherein the amino acid sequence is SEQ ID NO: 3, 6, or 9.

65. The method according to claim 63, wherein the amino acid sequence isSEQ ID NO: 54.

66. The method according to claim 63, wherein the modified APS polynucleotide is an APS1 polynucleotide comprising the deletion mutation of SEQ ID NO:130 resulting in a frameshift mutation.

67. The method according to any one of claims 60-62, said AGPase comprises a modified ADP-glucose pyrophosphorylase large subunit (APL) polynucleotide, a encoding the amino acid sequence that has at least 90% identity with SEQ ID NOs: 12, 15, 18, 27, 30, 33, 45, 48, 51, 57, 60, 63, 72, 75, 78, 84, 87, 90, 96, 99, 102, 108, 111, 114, 123, 126, and 129.

68. The method according to claim 67, wherein the amino acid sequence is to SEQ ID NO: 12, 15, or 18.

69. The method according to claim 67, wherein the amino acid sequence is SEQ ID NO: 57, 60, or 63.

70. The method according to any one of claims 60-69, wherein the plant is from the Solanaceae or Cucurbitaceae family.

71. The method of claim 60-70, wherein the plant is selected from a a tomato, eggplant, pepper, zucchini, cucumber, watermelon, pumpkin, squash, or melon.

72. The method according to any one of claims 60-71, wherein the plant is from the genus Solanum, Capsicum, Cucurbita, Cucumis, or Citrullus.

73. The method according to any one of claims 60-72, wherein the plant or plant cell is a tomato plant or tomato plant cell.

74. A fruit from the plant, or plant grown from the plant cell, according to any one of claims 60-73.

75. A method of imparting blossom end rot resistance to fruit from a plant comprising:
modifying a plant or a plant cell to reduce or eliminate the expression, stability, and/or activity of an ADP-glucose pyrophosphorylase (AGPase) subunit protein,
wherein said reduction or elimination of the expression, stability, and/or activity of AGPase protein is effective in reducing or eliminating starch synthesis and imparting blossom end rot resistance to fruit from the plant or a plant produced from the plant cell, as compared to fruit from a plant without the modification; and
selectively expressing a functional AGPase protein in non-fruit tissues.
